SEQUENCE LISTING (197 sequences)

SEQ ID NO: 1 - protein, 1283 aa, Artificial Sequence
  Description of Artificial Sequence: Synthetic polypeptide
MEENRSQKKCIWDELTNVYSVSKTLRFELKPLGETLKNIRKKGLIEEDKKRDEDFLEVKKIIDKYLSYFIDRNLDGSKNLIEEHQLKEIQDIYEKLKKNTTDENLKKDYASLQSKLRKEIFAQLKTKGHYKDFFGKQFIKKVLLDYYKEEDNKYDLLKKFENWNTYFTGFYENRKNIFTEKDISTSLTYRIVNDNLPKFLDNIAKYNELKNSLPIQEIEEEFKDYLQGMPLNVFFSLSNFKNCLNQKGIDTFNLLIGGRSPDGEKKIKGLNEYINELSQHSNDPKSIKRLKMMPLFKQILGENNTNSFQFEKIEYDRDLINRIDDFNKRLEEQDLYSNLYEIFKDLKDNDLRKIYIKNGKDITNISQQLFGDWDKLYKGLREYAEQDLFSRKNEIEKWLKRKYISIHELEKAIEKLKISQEFDKKLYENYLEKINYNENNPICGFLSTFKQKEKDLLEDIKTNYSNYLEISKKEFGEGDLLKEDYQRDVEIIKSYLDSLKELLHYIKPLYVDSKDTEDSKQQEVFELDANFYETFNELYFELKEIIPLYNKVRNYVTQKPFSTKKFKLNFENSTLLNGWDKNKERDNFSVILRKKNELGTYEYFLGIMSRGNNKIFENIEESNEDDSFEKMDYKLLPGPDKMLPKVFFSEKNISYYKPSEDILAIRNHSSHTKNGSPQEGFMKKEFNKDDCHKMIDFYKNALSIHPEWSNFEFNFKKTSFYEDTSEFFKDIADQGYQINFRNISSKDINQLVDEGKLYLFQIYNKDFSTNKSQKNRNSRKNLHTLYWEELFSPENLRDVVYKLNGEAEIFFREKSIEPKTEHPKNQEIKNKDPINGKKYSKFSYDLIKDKRYTEDKFLFHCPITMNFKAKGSKWDINKIVNSTIKENSKEINILSIDRGERHLAYWTLLNSKGEIVDQDSFNIIKEETIGRKTDYHEKLSEKEGDRDEARKNWKKIENIKELKEGYLSQVVHKLAKLAVEENAIIVFEDLNYGFKRGRFKIEKQVYQKFEKMLIEKFNYLMFKDREKNEIAGSLNTLQLTPQISSEKEKGRQTGVIFYTDPNYTSKIDPKTGFINLLYPKYESVEKSKNFFKKFESIKYNGEYFEFTFNYSNFYNDLNLTKKEWTICSYGDRIFSFRNPEKNNQFDTKTIYPTDELKSLFDKYYIEYESQKNILNEITKQSSSDFYKSLMFILSKILQLRNSIPNSEEDFILSCIKDKKGNFFDSRNANKNTEPANADSNGAYNIGIKGLMIIERIKNCPEDKKPNLTIKRDEFVNYVIGRNT

SEQ ID NO: 2 - protein, 1235 aa, Artificial Sequence
  Description of Artificial Sequence: Synthetic polypeptide
MARKKQLSGYRLHKQRVLFSSKEVIRTVKYPIVPIDKNNSQQIKILNQFKEKIINDDIKLKGDLNLNDYLEYSNQNRPPYTLFDFWLDSLKAGVIWRAKPLDVADFILTFYPSSTSPFNQVFNQNWENANDKIKKFFKKEEFKDIILSGPFRINKSVTSFENQLKKYLKEDFEKSKEAEDLISEIIDSFFDEKGNLKFNGEKQNEVWKEKFNIDKSLLEKSKPKGDLGNITFLIIPELIALDNDISLEQLISKREQWFLEKKLTKEEIKEKWLQEILGLEDNFNGFSNYFGNLFKNLQENNINKIFEALKTFFPELIQNKDKIFQALNYLSEKAKKLGNPSVVTSWADYRSIFGGKLKSWFSNFIKREKELNDQLENLKKGLESTRKYITEKKEKLSQYIDANQEVDELFLLISRLEEIIEERKIIQENEYELFDFFLSSLKKRLNFFYQNYLHEEDDESSVMDIKEFKEIYEKINKPVAFFGESAKKRNKEVIEKTIPIIEDGINIVLNLTKSLASDFDPLSTFNCFKRKNETEEDNFRKLLQFIFRKLQNSAVNSSRFTMNYISILQRELVNWSWKDFFKKKDKGRYVIYKSPFAKDPLTKIEIKEGNWLIKYRQVILELKDFLQQFSAEELLKDKNLLLDWIELSKNVLSHLLRFNKKEEFSVDNLNFENFKTAKNYINLFSLTNVNKEEYGFIIQSLFFSKLKAVATLYTKKSYLARYTFQVIDTDKKFPIFYQPKDNRIILKEIDLNSSDKSLSLPHRYLISLSRVEENKIRDPNFIHIYKESLNKVFLENEQLNNLFLLSSSPYQLQFLDRLLYKPHAWKDIDISLMEWSFVVEKEYKIEWDLETKKPKFYLKDNSRKNKLYLAIPFGIKSTKKDSVLSNVAKNRANYPILGVDVGEYGLAYCLILVDDNQIKVKKTGFIVDKNTAAIKDRFHQIQQKARHGIFDEIDNSVARIRENAIGHLRNQLHVVLITDQGASSVYEYQISNFETRSNKTIKIYDSVKRADVKVDSDADQQIHDHIWGKKADLVGKQLSAYASSYTCSKCHRSFYEIKKNDLEKSEITADQGNILIIKTTKGMVYGFSENKKYKDKSYNLKNTDEGLNEFRKLVKDFARPPVSYKCEVLNKFAPFMFNDKKFFEKFKKDRGNSAIFVCPFVGCQFVADADIQAAFMMALRGYFNFKGIVKTSKENNQGKNNKTTTVTGESYLKETIKLLNNLNFFPDDLFLVNKV

SEQ ID NO: 3 - protein, 1259 aa, Artificial Sequence
  Description of Artificial Sequence: Synthetic polypeptide
MHLSQTFTNKYQVSKTLRFELRPQGQTKEKFERWIAELRTENPSADNLIAEDEQRAVDYKEVKSIIDRFHRKVIEESLEGLKLKGLSEYEELYFKREKEDIDLKEIENLQIQMRKQIREAFVEHPVFKDLFKKELIQVHLKEWLTDQQEIDLVAKFEKFTTYFGGFHENRQNVYSPDAKATAVGYRMIHENLPKFLDNRRIFNKIIKAHEELDFSSIDSELEELLQGTTVEEVFSLEFYNETLTQTGIDIYNHVLGGYSSETGQKIQGVNEKINLYRQKNGLKARELPNLKPLFKQILSESQTASFVIEQIESESDLLDRLDNFHTLITSFEFQGRNQVNVMTELKHMLAALDSYEHEQVYFKNGPSLTQLSQKMFGQWGVIHKALEYYYEQEQNPLQGKKLTKKYENDKEKWLKNKQFNLSLLQKAIDVYVPTIDTIEPVSIVETLSTLEDKEGADLGTEVDNAYEKVAELIEQKTLSESYAQKKKEKQVIKEYLDGLMSLLHSVKPFYTTEVDIEKDAGFYGLFEPLYEQLNLVIPIYNLVRNYLTQKPYSTEKFKLNFENNTLLDGWDQNKEKANTCVLLRKEGNYYLAVMHKNHNTVFEELPQNENATYEKVIYKLLPGANKMLPKVFFSKKNIDYYKPKEELLEKYKLGTHKKGSNFNLKDCHALIDFFKDSISKHPDWAQFNFEFSQTKTYEDLSHFYREVEHQGYKINYAKVDVSYINQLVDDGRIFLFQIYNKDFSPYSKGKPNLHTMYWRAVFDEKNLADTVYKLNGKAEIFFREKSLNYSKEIMEKGHHRDELKDKFSYPIIKDKRFALDKFQFHVPLTMNFKAGSNPNLNDRALDFLKDNPDIKIIGLDRGERHLLYLSLIDQKGNIIEQYTLNEIVSKHKDKTFKKDYHELLDKKEKGRDDARKNWDVIETIKELKEGYLSQVVHKIAQMMIEHNSIVVLEDLNAGFKRGRHKVEKQVYQKFEKMLIDKLNYLVFKDHDKEKPGGLLNALQLTNKFESFQKLGKQSGLLFYVPAALTSKIDPATGFTNFLRPKHESIPKSQSFIAGFTRIHFNSEKEYFEFKFDLKNIPNTRFPDDTKTEWTVCTTNVPRYWWNKSLNEGKGGQEKVLVTQRLQDLLARYDLGYATGENLKEDILTIEDASFYKEFLWLLNVTVSLRHNNGKHGELEEDAIISPVANAQGEFFNSSEAKSSAPKDADANGAYHIALKGLWALRTINAHDKKEWRGIKLAISNKEWLQFVQQKPFLKP

SEQ ID NO: 4 - protein, 1336 aa, Artificial Sequence
  Description of Artificial Sequence: Synthetic polypeptide
MKQEKKTEKSVFSDFTNKYALSKTLRFELKPVGETLENMKDAFGYDKKMQTFLKDQEIEDAYQNLKPILDRIHEEFITQSLESEQAKQIPFHIYEKSYRKKSEITLKQFETVEKKIREYFDEAYKQTAQVWKQNAPKDKKGKGVFTKDSHKLLTEVGVLEYIRQNTEKFSDILPKSEIEQHLNVFSGFFTYFQGFSQNRENYYTTKDEKATAVATRVVSENLPKFCDNILTFENKKEAYLALYQSLAEKGKTLQIKDGSSGKMKSLEGVDEAMFSIHHFNECLSQREIEKYNEAIANANYLINLYNQLQDDKKNKLKLFKTLYKQIGCGDKETFIEKITHYTEEEAQKARKEKKEKAISLEQELKEFSSLGSKYFFGISENEFIRTVEDFRKYLLEEKEDYAGVYWSKQAINNISGKYFSNWHALKDILKEKKVFSTSASKDESVSIPEIIELKQLFEVLDGIEKWEVPDNFFKKTLTEEVSKDHRDFQKNAKRKEIIKSSQKPSEALLRMMFDDMVDLREKFLSKKEDILENTNYTTQERKDDIKEWMDSGLRIIQILKYFSVQEKKIKGTPFDAKIKEGLDTLLLSNEVDWFTRYDRVRSFLTKKPQDDAKENKLKLNFENSTLAGGWDVNKESDNSCIILKEEEKTFLAVIAKSKGKEKNNALFRKTEQNPLFSIENAETMKKMEYKLLPGPNKMLPKCLFPKSNPKKYGATETVLDVYKKGSFKKNEENFSKKDLYTVIDFYKEALKRYEGWNCFEFHFKKTSEYNDIGEFYLDVEKKGYTLDFVDINRNVLGQYVEDGRVYLFEIRNKDWNTLPDGSKKSGNTNLHTMYWKALFQDRENRPKLNGEAEIFYRKALSKDEIKKKKDKHEKEVIENYRFSKEKFLFHVPITLNFCLKDYKINDDINEKLLENENVCFLGIDRGEKHLAYYSIVDNEGNILEQDTLNTINGKDYNTLLEERSEEMDTARKSWQTIGTIKELKDGYISQVIRKIVDLSLRYNAFIVLEDLNVGFKQGRQKIEKSVYQKLELALAKKLNFLVEKSAHQGEMGSVTKALQLTPPVNTFGDMEKRKQFGIMLYTRANYTSQTDPATGWRKTIYLKRGGEKLIRENIIQSFDDMYFDGKDYVFSYTEKFGKDKNNQRSGRSWKLYSGKDGISLDRFRGKRGKEFNEWSVETIDIAGILNELFEDFDKNISLLEQIQQGKDPKKINEHTAYETLRFVIDSIQQIRNSGEKGDERNSDFLHSPVRNTEGEHYDSRIYLDREKEGIVTDLPISGDANGAYNIARKGILMKEHLKRDLSEYISDEEWSVWLSGKNRWEKWMQENEKDLRKKKK

SEQ ID NO: 5 - protein, 1146 aa, Artificial Sequence
  Description of Artificial Sequence: Synthetic polypeptide
MKNNRTKHLHPTGYQLASERIKQAPLNKNSKYIVTVKYPLKGDLKGKLESELIEQSFRDYAYAYGIPTLKESKPQVSLIDFYIECLRMGAFFQPSSAKLQDLASGGKLQALIKKNIPDHILVKLNMLEFVDGITADFRKMEQEEPATFRKKIAKWFKDDTDPYIDQVVEIYLQNGQSQQTQSAESAFFYRPKKNPSNLTFYLHPEILVDPSESNPQKVVFESVRQIYTALNNQLQPPEKKREDFDLELIGLDKQANALSNFFNNVFNRLQKDDVQSLMAEILDLSELWRGKEQELEQRLIHLSSVAKQVGNPALGKSWADYRAMFSGRIKSWYKNTVNHLKAREEQLPNLKEAVEVVIADVRQVVELITNKSFDERDNSNRTELLFHFLESCQALLDALDQNNEDVCFQLHAELTRDFNLVLQRYAQEFLTLENSKKKKKQFAEDSAEALELIRPKYAKLFSRLRPQPAFFGEQRAKLVDRYSEAAKQLFQLLTFLQQLILDLYALPRGDALGEETLLQIVDKVVKRKNNANTINHQQLFKDLFTQAIIRPYTKDEKVAYFINPNASRLRLRKLEKSWRLPDVELVQMIESTLLKSFNLSQEAYSHADSESLIDAIESSKTLVAVLLLTRKSTQYSFDFEKIPSETLRFKINRLDKKNRVQYLQRATSFIGTELRGYISLISRSEVIDRATVQLSNSDKMFTPVRTKDNRWKIALNHEKAAIGLDQEVEKFTKSGVKREVLKHQTLDIKTSRYQLQFLEWLHKTPKKKQHLNIALNEPSLIAEKKYRINWTVQNQILVPEYVLLESGVFLSIPFTISPAKDNNKSFSRYLGLDLGEFGVAWAVLGIKDNRPYLVQTGMLQDPQLRAIANEVAVMKARQVTGTFGVPSSRLQRLRESAVHSLVNQIHSLVLRYGAKMVFERQVDAFQTGSNRVKKIYASLKQGNIFGRKEIDKSNYKRYWSYRDGHFMGSEVSSWGTSYFCPHCREFLHDLPKEKDAYELVKDSPEELTRLRVYSVKQTGEKYYGYVEGNSSPKEQVLAFARPPYQSDALLLLSKQGKNLNLSQSLKTERGGQAVFVCPKFSCLRTYDADKQAAVNIAMRKWAEDVFIATKGKPPKQRDENYFRMRKDFERKLYKDLNEYPTVKMGE

SEQ ID NO: 6 - protein, 1167 aa, Artificial Sequence
  Description of Artificial Sequence: Synthetic polypeptide
MARKDKYRGLTGYRLHQKRLERSGKQGIRTIKYPLVGATEEHHEQFVSDVIHDYNAQVGALNLPEWLAQYRGEQTFYSLFDLWLDLLRAGFVCAPSSARLMERVCWLADLPSPRAQLRDQMQEVNPDFYTALSENGFHHFVDTVVLGKEMRSSKSERSFVRDLTTCATDAAQEYAEREARTIYHALYGSDRTEQERYWREHYGVDKTLFQPTTRRNFAAYPVPALQLSPDAAPGALLQRYRSLVQTQLSAQQAERVATQETQLLEDMLGIDNNANALSNVFNEFLREVRTETGRAAIADDMQQFSRAWDGRRSELEERLRWLGERAAQLPAQPRLANSWADYRTSVAGKLQSWVSNVARQEHVIRPRLEQQRSELDDLAERLRALSDEETGLPATVEQAQAALDAALAAEQSDESTLMVYRDALADVRAALNEGQHTLQMHEHGIEHVDTDSSWASDTWPTLHQPVPQVPQFPGVTKAYAYTKYVHALELLRSGAAVLERAAADASEREAVQLSREEMLRRLTNVAQQYARCNSQRFRDLIGGVFQRHEVLLNDVVERGAVYYQSPRARNKKPLVELSHTDEQLHAVITDLVWKCAPYWERMWGQIEEVVDAIDFERVRLGMLCALYPDTTADISDVSETLFTRAGGYQRAYGTELTGTTLSNCIQRVILAEMKGAAQRMSREWFVVRYTVQIVKADELYPLIYQPGSTGGRGTWHITDRQNVRRSAADTPPVYRKVGKNLPHDTALAGFDGAEVTDTQRLLSIRSSRYQLQFLQDQLHAGSEHMRRRFSWSIAEYSFICEDTYTAAWDTERGTVSLERQPSARRLFVSIPFQLRRLEAADGRSSYQPKSGLPYSYLLGLDVGEYGIAYCLLEPETGEWRTSGFFADDAIRKIRQYVSRQKEAQVRSTFSAPSSELARIRENAITALRNRVHDLTVRYDARPVYEFNISNFESGSNRVAKIYRSVKTADVHADNDADQAERDLVWGSASKLTGSEIGAYGTSYVCSKCHASPYTAIQPMQQSAYEWEWVGQQQRIVRIYTPENGAALGHIDIRQYKPSDTLPSVDALRFLKAYARPPLEALVQRSGFTDQDTIDRLHAYVQERGDSAVYTCPFCEHTADCDVQAALIVAVKYAIKQHGSPSGEKGEVTLEDVSAYLRGHEVQPVSFA

SEQ ID NO: 7 - protein, 1245 aa, Artificial Sequence
  Description of Artificial Sequence: Synthetic polypeptide
MRRQLEDFANLYEISKTLRFELRPIGKTRKMLEENKVFEKDEAVAQNYQEAKKWLDKLHRDFISRSLEDLKINSELLEEHKQAYFDYKKEKNSSNRNNFEEKSKKLRKEILLNFCQKGEELRDNYLREIKDEKIKKRVRKLRNLDILFKVEVFDFLKQRYPEAVVDEKSIFDAFNRFSTYFTGFHETRKNFYKDDGTATAIPTRIVNENLPKFLDNLEVYNRYYKEGIGDLFTGEEKNIFNLEFFNDCFSQREIDSYNRIISEINLKINQKRQTAENKKNFPFLKTLFKQILGEEEKQETESLDYIEITRDEDVFPALKSFVEENERQTPRANKLFNRLIQDQKEQKGGFDISNVFVAGRFINQISNKYFADWNTIRSIFIEKGKKKLPEFVSLQELKEKLQSIEIEKSELFREKYKDIYKNRGDNFIIFLEIWQKEFEESLKRYRESLEETKQMLEQQEGYQSKESSEQKNSIRRYCENALSIYQMIKYFSLEKGKERVWNPDKLEEDPGFYELFKDYYQDAHTWQYYNEFRNYLTKKPYSQDKVKLNFGSGTLLQGWPDSPEGNTQYKGFIFKKNKKYFLGITNYPKMFNEKRHPEAYDNDIDPYYKMIYKQLDSKTIFGSLYLGKFGNKYKEDKKRMVDFKLQNRIRAILKEKVEFFPRLQTIIDKIENHKYSNTKDIAVDISKIKLYNIFFIETNSLYVEQGKYEIDNNTKNLYLFEIYNKDFAKKAEGKKNLHTYYWEEIFSQRNQDNPIIKLNGQAEVFFRRASLDPEVDEERKAPREVVNKERYTEDKMFFHCPLTLNFAKGRADGFSIKAREYLLENPEVNIIGIDRGEKHLAYYSVADQEGNILEIDSLNKINEVDYHKKLDKLEKARDEARKTWQDIAKIKEMKQGYISQVVKKICDLMIKHNAIVVFEDLNLGFKCGRFAIEKQVYQNLELALAKKLNYLVFKEREAEELGSFRHAFQLTPQISNFKDIKKQCGFMFYIPARYTSAICPNCGFRKNISTPVDKKAKNKEYLEKFQISYEQDRFKFAYKKRDVLERGRGNPGQNSRRLFEEKASKDDFIFYSDVSRLQFQRNKDNRGGETKWREPNEELKRIFKENGIDINKDINKQIKEGDFENDAFYKRIIHTIRLILQLRNAITKKDEQGNEIEEESRDFIQCPSCHFHSENNLLALSEKYKGDEPFQFNGDANGAYNIARKGSLILSKISNFNKTEGDLSKMDNQDLTITQEEWDKFAQNK

SEQ ID NO: 8 - protein, 1148 aa, Artificial Sequence
  Description of Artificial Sequence: Synthetic polypeptide
MTENISTEKQTAYKIQNSSDKHFFASFLNLAVNNVENAFDEFAKRLGVSNSNKKGERYKPDESIKQFFKPELSLTDWEKRVDMLEQYFPLVSYLKGNVTDNNEKDSKSKILKCDFSSHDEMKKAFANYLTYLVKALDDLRNYYTHFYHDPIKFKPEDKKFYEFLDELFVEVIKDVRKKKKKSDKTKEALKDELEIEFEERMKDKSAALEKMDKDAGKKVKNRSEDELRNAVMNDAFKHLIAKDKDEYSLIERYQAFPENLDAPISEKSLMFLCSCFLSRRDMELFKARITGFKGKMVEGEDSLKYMATHWVYNYLNFKGLKRKINTRFEKENLLFQIVDELSKVPDCLYRVIKDKNEFLLDINKFYKQTKGEAESPENEEVVNPIIRKRFEDKFNYFALRYLDEFAGFENLKFQIYAGNYLHHKQEKTSAQTQLKTDRKIKEKINVFGKLSDVNKAKANFFANKTEDSDMDEGLEEYPNPSYNINGGSILIHLNLNKYRYGQEFHELKQLRIEKEKRGENKTDKISIIKDLFEDNTEIKEEDWVFPVALLSLNELPALLYEMLVNKKSSKDIEQIIADRIVSHYKKIKDFEGTADELKDKNLPVNLRKAFGADDKNTDKLENAITKDIEAGEDKLQLIKENTREMRSNNRKYVFYLKEKGEEATWLAKDIKRFMPENAKNQWKSYNHNELQKGLAYYELERQNVLALLESKWDMDSCHPHWGEDLKELFITHSRFDDFYKAYMLCRQGFLEQFKTLVIRNKSDKKLLNKVLKDVFIPYKKRFFVINSLENEKKALLSHPIVLPRGLFDNKPTFIKGVSLENDPSRFANWFAYLRQEAKNDHQVFYDFERDYVKAFSELKDKSKYNNNKHFNFKVDSEIRMCLQNDLVLKLIVKKLFKGIFDVDENIKLNDFYLEKTEVAKQREQALDQNKRLKGDDGDVIYKEDHLFRKTFAKDFLNGKLHFDKFKLKDFGKALVFAADEKVKTLVSYSENAWTQEELQKELHTNTDSYERIRQDEFFKKIHELEESIWQKHKHEREKLQDKSGNENFNNYVKVGVLEKLNDSFKDEFENLYKDKKNKRIQKLRQCNHVVQKAYCLVQLRNKFSHNQLPPKQLFDFMTETLAEKDKQTYSRYFMDVTDKMVQEFKPLV

SEQ ID NO: 9 - protein, 1138 aa, Artificial Sequence
  Description of Artificial Sequence: Synthetic polypeptide
METQIVNKKRTLKDDPQYFGTYLNMARHNIFLIENHIAQKFEKNKLGVVKSDEHIASRQFFDAAFKNNKLANSKQIFNAFTRFIHVAKIFDNDLLPKSEKQEEGFQQDSIDFNLLSETFFSCFKELNQFRNNFSHYYHIENEEKRNLFVSETLKYFVIKAYEKAIAYAEQRFKDVFKHEHFNIARNKKLFTLHQEFTRDGLVFFCCLFLEKEYAFHFINKIIGFKDTRTAEFKATREVFSVFCVTLPHNRFISEDPAQAYILDALNYLHRCPTELYNNLSEDAKKHFQPTLSYEAVQNIQGSSVNNEQLPIEDFDDYIQSITTQKRNTDRFPFFALKYLDNKESFKPLFHLHLGKLLLKSYKKNLLGNEEDRFIVESFTTFGTLENFQLSNIEEENKEEKVREITQLKKEITIEQYAPKYHIANNKIALNLSNNKYYNGNFLSFHPEVFLSIHELPKVALLEHLLPGKATQLIENFVNLNSSHILNSQFIEEVKSKLTFTRPLKKQFHKDKLTIYNYTLQQLNNKINEIIQFIDDNKEHADDETKNQIKNKKSELKNLYYNRYVVQVVDRKQQLDAILKTYNLNHKQIPERIINYWLQIKEVKDDTTLKNKIKAEKEECKQRLKDLANLKGPKIGEMATFLAKDIIHLVIDLQVKKKITTFYYDRLQECLALYADIEKQQTFKRICSELGLLDALKGHPFLNQIILGNYSKTKDFYRAYLQQKGTNTIEKYDYNRKKIVESNWMYTTFYNVENKQTIISIPNNKPVPYSYKQWQAPQTDFNKWLSNTSKGIDKQQPKPIDLPTNLFDETLNSALQQKLQNPLPNEKANYTALLKAWMPQSQPFYNMPRSYMVYDNEVNFTPGTQATYKGYFEKTIQKVLRQKNEQIKKDNLKAIKKKPFYTASQILAVCNNAITENEKLIRFYETKDRILLLIVQELSGMQMCLQKMDIKSQQSPLNEIIEIKEVIHQKTITAQRKRKDYTILKKLEKDKRLPNLLQYFDEDTIPFDTINKELFHYNQSREKIFDSSFLLEKTIVEKLQQNQSMHILTTMQEEKNKKEGTDVKNIQFDIYTQWLQENKFISQTEADFLLTVRNKFSHNQFPEKIKIEKEVTFDENQNKASQICENYHKKIQAIIAQLN

SEQ ID NO: 10 - protein, 1093 aa, Artificial Sequence
  Description of Artificial Sequence: Synthetic polypeptide
MVNVNKRTLTGDPQYFGGYLNLARLNVFAISNHIAEKINPFLKKGKVGVLQDDENIPDSFICNKIKEKPNLFYTQLVRFFPIARVYDSDRLPKEEKLLTKCEGIDYSLLTGDMKICFSELNDFRNDYSHYFSIKTGTDRKVEISERLSDFLMTNYLRAIEYTKVRFKDVYNDSHFQIASKRILVDENNIITQDGLVFFMCIFLERESAFHFINKIIGFKDTRSLDFKAMREVFSAFCITLPHDKFISDDGKQAFILDLLNELNRCPKELFENISSEEKKQFQPNVSESAADIEENSIPADLPEEDFEEYIQSIISKKRKTDRFPYFAVKYLDEKTNINFHLNLGKIELVTRKKKFLGGEEDRDIIEDAKVFGKLGEYADERAVSKRLGMEFQLFNPHYQIENNKIGFSFSPIECSIKNVNGKPNLKLNPPNAFLSINEMPKVVLLEILQRGKVTEIIKEFIQASTDKILNREFIEEVKSKLDFKKPFNRSFSKKRNSAYGPKGLQILTERRTSLNLILKEHNLNDKQIPGRILDYWMNIVDVTDDKAIANRIQAMKKDCRDRLKQKAKNKAPKIGEMATFLARDIVDMVIDENVKKKITSFYYDKMQECLALYGDAEKKELFIRICGEELNLFDKGIGHPFLFELNLQSINKTSELYEKYLIKKGTAEHIKWNERTKKNYKVETSWLYTNFYNKIWNEEKKKMETKLKLPEDLSKLPFSIRNLTKEKSSLDKWLNNVTKGCLEKDRTKPIDLPTNIFDETLVKIIREKLNDKQVSYKDTDKYSKLLELWKGGDTQPFYNAEREYTVYEEKVRFRLGEKNSFKEYFKDALEKVFKKESSKRQSERGKPPIQKKDLLTVFNDAITENEKVVRFYQTKDRVMLMMVKDLMGAELDFKLSEIYPLSEKSPLNIEEEIEQRVEGKLSYDGDGNYIKGGKESITKIIYARRKRKDFTVFKKLTFDKRLPELFEYYAEERIPYEKLKAELDEYNKHRDMVFDVVFELEKKIMDKPEALREMEDVGDKNVRHKPYLNWLKKRKVIDKKQYALLNAIRNSFSHNQYPPRMIVENKIKIKAGGITPQIFERYKEEIEIIMNKI

SEQ ID NO: 11 - protein, 1236 aa, Artificial Sequence
  Description of Artificial Sequence: Synthetic polypeptide
MRIIRPYGTSATEPDAQDPAKRRRTLRRKLDAPGATTVTERDLGAFARRHDVLVIGQWISTIDKIASKPAGFKKPGAEQRALRRRLGEAAWRHIVAHGLLPGRAETPSLETLWWMRLEPYPTGDAKYGRDPKGRWYARFVGEIEPEEIDADAVVERIAEHLYAHEHPIHPGLPTRREGRIAHRAASIQAAVPKAEPRAARATWTDAHWTIYAEAGDVAAVIRAAAEEVQAPPPPDDKAAKGKRRWVGPDVAGKALFEHWQRVFVDPETEAVLSVGEVKARIENGDDRLRALFELHEEVRGAYRRLLKRHRKAVRGSSGKPTRTSDVARLLPSSMDALQRLLAAQRDNRDVNALIRFGKVIHYEAAEPTSEVPPDDDGRPRHDEPAHVLDDWPDAARVARSRFWTSDGQAEIKANEAFVRIWRRVLALMHRTATDWAMPEADDDFTMARVLERAVGEDFDQARHRRKVELLFGARADLFRGDGADDALDREVLRFALEHLRSLRNKSFHFVGVGGFKAVLTGANEAPADGAAPAQARALWAQDQRERAKQLGKVLQGVQAGDYLEGNELRALFDDLVAAMTTPSDLPLPRFKRVLLRAENIRDKRQDDPHLPAPANRLDLEEPARLCQYTALKLVYERPFRRWLADADAAKVRGYVEGAARRSTDAARKLNDPKDEAKRERVRSKAERIANLAPDATMRDFVRTLMRETASEMRVQRGYESDAENARDQARYIEDLLRDVVALAFLDYFRDAKFGFLLEIAADRTVDPAKRLDPTTLEAPEADVSAEPWQVALYFVSHLAPVDDIALLLHQLRKFDILAEKRGAGTDDALRAQVEAVIKVFDLYLDMHDAKFEGGRGLAGLEDFAQLFESRELFEELVAKPVGQDDSERVPVRGLREIARYGHLPPLLPIFQKRRITEEDAREFRERGGTIADRQKERQALHAEWAEKPKAFANHSVAEYTRALRDVAQHRHCANHVSLTAHVRLHRLLMGVLGRLLDFSGLFERDLYFAALALVHENGLRTEEAFGKRCAYLIGQGRILAAIRHLDAEIQKELGGLFLLDGATKVIRNHFAHFKMLQPSRADAAALNLTSEVNGCRQLMRYDRKLKNAVTKAVIEFLEREGLDIRWTWNDAHELSVPTLKTRAAKHLGGRAIAERREDGAVPDVRDGFPIQEALHAAGYVEMTAALFAGHAAPIRNEICALDLERIDWRRPQRRDGSKGKGKGKGKNRHPAPNKAQ

SEQ ID NO: 12 - protein, 1092 aa, Artificial Sequence
  Description of Artificial Sequence: Synthetic polypeptide
MQKHQIMDKGNAEGNYRHFDEEADKPFYAAYLNTAKQNIFLVLRDISEKLDLGFNFDSDDQLFSVELWKQLKTGKRPNLTQKIIAHLKQQLPFLEIAAIANARKQSNDHKAQPQPEDYYHILEHWVSQLLDYCNYYTHATHNSVNMARVIIGGMLDVFDSARRRVKDRFSLMPADVEHLVRLGPKGGQNDRFHYSFLDKQGRLTEKGFLFFTSLWLKKKDAQEFLKKHEGFKQSQENADKATLEAFTIFGIKLPKPRLTSDLGDQGLFMDMVNELKRCPEELYSLLSKEDQATFKPHDSEEATNDDENPPELKRNQNRFYYFALRYLENAFQNLRFQIDLGNYCFKTYEQEIEQVAYKRRWFKRITAFGRLTDYKEHNQPMEWEEKLLKVPDRDKPDTYITDTTPHYHLNENNIGLKKVTDKDKVWPEIPKKENGKKPEGNPPDFWLSIYELPAVVFYQILYEKGLAQFSAESIIEIYAGEIQKLLDDVKVGNIASGYSKEQLQTELENRALHISYIPKPVIKYLLGEDEWSFEEKAAARLQALKAENDQLLKKVKRKQLHFRQKPSNKDFRIMKPEEIADFLARDMIWLQQPDNKEKNKPNKTEFHHLQGKLTYFRKYKMTLLKTFRRCNLVDAPNAHPFLNQINLLACKGLLNFYVTYLEHRKAFLEQCTKEQDYAAYHFLKVKRDKDAIATLIEKQQDAVCNLPRGLFKQPIMEALKNSDETRGLAASLEKMDRANVAFIIQNYFHEVQQDDNQAFYDYKRSYELLNKLYDQRKTNDRSPLPSVFFSTRELEEKKDEIPQKLADKVQSRIEKNSIKDEKEKERIQQKYRKRYKQFTENEKQIRFFKTCDMVLFLMADQMYRSGDPIGLHDNNDNTAQGITGMGEAYKLKNIRPDAERSILSHETLVKIPVYFNNASESRSKTIVRERMKIKNYGDFRAFLKDRRLTGLLPYIEADEIVYEALKTEFEAFHDARIEVFEKILEFEKIFLIKVRPKAKKKRYIPHELLLQQNAIDLPSYQIKNMIALHHSFNHNQYPDAKQFGEYIDGSNFNQLKLYTADNQEVMAHSIIVQLKKLALWYYDKAIKLTNAS

SEQ ID NO: 13 - protein, 1053 aa, Artificial Sequence
  Description of Artificial Sequence: Synthetic polypeptide
MTLPDKQQSTIYSMDRSEDKYFFALYLNIAQNNVDKVLKEFDSWFNSLNETSQGKYNSAQAKWLDNRLPGSDSDVLEAKERLVYLRRFFPFIETEFTTKEYHGYREKLLMLFERLNDFRNFFTHVHYERNELEFSRNKKMFEFLNEVKEIALNKLNQHPYYLDDNILNHLHDPDQRFNFQKENNIKDAINFFVCLFLENKHAHEYLKKQKGYKSSHNPEHRATLKTYTFYSIKLPRPVFESRDMKLRLILDALNELKKCPKQLYDHLSEKHQKLCQVESVKQKENEESGETEEIKEYIPFIRHEDKFPYYALRFIDDLELLKDIRFKIKRGLGKEFFHTHETATQPVVRNKKVFTFRRFLEVYEGERKEPDNNLWHPAPAYAFEKDGNIKVKITKNEETSKSKDDTSSDDIAYAELSVYELRNLVYCCLNGKKDAANNIIRDYVFNYKAFLKDLENKDFSEIDDYTAQLEERKQQLQNKLSEYNLQLHQLPKKIRKILLDEKIQDYKSHTIQKIKDRQEENKRILGKIKAQKQMSKENDKDSQQKNTLKTGQLASELANDIQNYLPENYKLELFQYRDLQKQLAYYRRKEIYILLNQNYALTYHEQQDRNENFNDLYYKKKHPFLHHVLTRKDNDDIFSFAFNYFKSKEIWLEKVRKKVIGLNDTDIPKYSELFYYFKPGTSVNEKGEKIYYRKYDDHYLNKLIQRHLKQDHVINIPRGILNQFICPEKESYEQKNNPIQKIADQYPSTQDFYKFPRFYHPTGEVLTVEDINYKLVELSKDKDHPHNNDKKEHKKAYNQLKKYLKKEKTIRYIQSCDRVLLEMIKYYLNNYFKKSNEEFELDLTDIELRDLFKYDETNESIHNKLDQKMITLKFHLNGQSFLAEDKLNNFGKLHRYIYDERFISIFKYKGNKAFEGVKTESIYSQLEKILEAFAKEQLELFEYVQQFEKTITTNFENKVNQKRTEENARREKNGKPLISEHYFPISILLSLTEEWGFISGKNRNFINTARNSAAHNKLDDKYIEMLKDREYENDYFGAASKIFNDLTEKIRTA

SEQ ID NO: 14 - protein, 1163 aa, Artificial Sequence
  Description of Artificial Sequence: Synthetic polypeptide
MTTIENFRKYNADKSFKNIFDFKGEIAPIAEKSSRNLELKLKNKVGVETSVHYFAIGHAFKQIDKEAVFDYIYDEETDSKKPHRFTSLKQFDEQFCKELKNIVSTIRNINSHYIHDFGQIKCDTLSLQLITFLKESFELAVIQTYLKSKESTKDAMTTQDFFDAPDKDKKIVEFLKERFYAIDSEKKNLESYQNHINRSKYFGTLTKEQAIETILFGEVVDPNFKWKLNETHIAFPISVGKYLSYHACLFMLSMFLYKHEAEQLISKIKGFKKSKNDEDKLKRNIFTFFSKKFSSEDIKSEQAHLVKFRDIVQYLNHYPLDWNKYIELESAYPSMTDKLKAKIIEMEIDRSYPNFVGNTRFHTYIKFELWGKKFFGNKIFKEYCDCSFTPKELEEFKYEKDTCGKVKDAELKLKEKHLLKHDEIKKLEDKIEENKDKPNNITLTLDTRIKKNLLFTSYGRNQDRFMQFATRYLAETNYFGKDAQFKMYRFFSSVDNTNEIESQKEKLDKKLINKKQFDNLRFHDGRLTYFATFKEHLVRYENWDTPFVEENNAVQVQITFNYEEILKDTNQTILVYITKVISIQRSLMVYFLEDALKSNTLANSEGVGVKLLFNYYMHHKKEFAENKHELENNDKESIDNTYKKIFPKRLINKFVAVSPNDPKQQSVYESILEKAKKSEERYKDLRAKAEKDKRLEDFDKRNKGKQFKLQFVRKAWHLMYFRDIYNLYAIDGKPENHHKHLHITREEFNNFCRYMFAFDEVPQYKLLLKNMLAEKHFLDNKAFETLFDSSHDLNSMYCKTKEKFKVWMSQPKETSNDKEHYTLANYEKFFKDKMFYINLSHFRDFLKEKKRFIIANDKIVFKSLENNQYLMQDYYIEETPAKEKYKTKEEYKANKNLYNELRKSRLEDALLYEMAMHYLGMEKDITKNAKVPVQKILSQDVSFEIKDLKNITNYTLSVPFKKLESYLGLMAFKEKQEQEYKGSYMINLVEYLKKIEQDKDTKKEIKQIWNDINGNKKLSLDQLNKFDAHIISNSIKFTRVAILFEQYFIVKHNHSIIKDNRISFEEIEEIKEYFVKLTRNKAFHFNIPEKPYSSLLKEIEKRFIQKEVKIQNPKSFDEIKLNEKYICSAFLNSLYDVYFNFKEKDEKKKRYDAEQKYFTAIIA

SEQ ID NO: 15 - protein, 1124 aa, Artificial Sequence
  Description of Artificial Sequence: Synthetic polypeptide
METTQTSENKRRSLATDPQYFGGYLNMARLNIYNINNYLAEEFGLSQLPEDGYIKNSFLCNQKQTKLNWNRVFSKAVTFLPILKVFDSESLPKSEKEDKSTPETGKDFAKMADSLKVLFSEIQEFRNDYSHYYSTEKGTDRKITISNELADFLKFNYKRAIEYTRVRFKDVYTDDDFNVAANKKMVIGGVITTEGLVFLTSMFLEREYAFQFIGKITGLKGTQYVGFRAFRDVLMAFCIKLPHEKLKSDDFIQSFTLDIINELNRCPKTLYNVITEEEKRKFRPQIEPEKIDNLLKNSGIELEEYDENFDDYVESLTRKIRHENRFNYFALRYIDENKIFGKYRFQIDLGKLVIDEYPKKFFNEEVQRRIIENAKAFDKLSDLVDETAILKKIDIQNHQVYFEPFAPHYNTENNKIALLSKSDIARVRKVKTKTGVERKNLFQPLPEAFLSCAELYKIVLLEYLKPGEAEKLVTDFILANNSKLMNMQFIELVKKQMPGWIVFQKETDTKSRLAYSQINFNELLSRKSQLNKVLAEHNLNDKQIPSKILEFWLNISDVKQQFTTGERIKLIKRDCMKRLKALKKFKTTGKGKIPKIGEMATFLAKDIVDMVIGKEKKQKITSFYYDKMQECLALYADPEKKKTFIHIITHELGLYEKDGHPFLNRINFNELRYTRDIYEKYLEEKGEKMVKFYNARRGNYTEKDKSWLRETFYTLVEKEIKGKKRIMTEVVLPSDKSKIPFTLLQLEEKTTYSLADWLQNITKGKEHGDGKKPVNLPTNLFDETITSLLKTELDNKQALYPENAKMNELFKLWWMGRGDGVQHFYDAEREYFVFEQPVKFKPGSKAKFSDYYCIALTKAFKEKEKTATKERKQAPELDEVEKTFQQAIAGTEKEIRELQEEDRVCALMLEKLISREKHITVKLESIENLLKESVVVKQTVNGKLYFDENGNEIKDKSNPVITKTIVDKRKGKDYGLLRKFANDRRVPELFEYFSGEEIPLEQLKKELDGYNIAKHLVFDVVFRLEEKLIKSNRNEIISYFTDDKGNAKGGNIQHLPYLNLLKEKDLVTPGEMAFLNMVRNCFSHNQFPKKSIMKKVVKPGENNFAKKIADIYNEKIEALILKLA

SEQ ID NO: 16 - protein, 1091 aa, Artificial Sequence
  Description of Artificial Sequence: Synthetic polypeptide
MSDSQLKPRYTLGLDLGVSSIGWAMIEPVDTAGPAKIVRSGVHLFDAGVEGSEDDIEQGREKARAAPRRDARQQRRQTWRRAARKRKLLRLLIRARLLPDSETGLQTPEEIDHYLKSVDADLRVTWEQDIDHRAHQLLPYRLRAEAIRRRLEPYEIGRALYHLAQRRGFLSNRKTDDDGGDGDDDTGAVKQGIAELEKRMDQAGAETLGEYFASLDPTDGASRRIRGRWTARPMYEHEFDRIWSEQAGHHSGRMTDEARQQIRHAIFFQRPLKSQRHLIGRCSLISKKRRAPMAHRLFQRFRLRQKVNDLQIIPCRRVEVDAVDKKTGEVKIDPKTDQPKRVKRWVPDPTQPPRPLTDDERAAALERLEHGDATFHQLRQAGAAPKASRFNFETEGESRLPGLRTDEKLREIFGDRWDAMDERVKDAVVEDCLSIVRGDTMERRGREAWGLSADEARAFARVKLEEGYARLSRAAMRRLMPHLRNGVPFASARKQEFPGSFATNPTVDTLPPLDKAFNEPVSPAVARALSELRGVVNAIIRRHGKPAHIRIELARDLKRGRKRRDAISRQIAARRKQREAAAERLIERYPHLGASARDVSHIDVLKVVLADECRWICPFTGRAFGWTDVFGPSPTIDIEHIWPFSRSLDNSYLNKTLCDVNENRKIKRNQMPTEAYGPDRLDQILQRVSRFTGDAAQIKLERFRAESIPADFTNRHLTESRYISTKAAEYLALLYGGLADDERNRRIHVTTGGLTGWLRREWGMNAILSDDDEKDRSDHRHHAVDALVVAFTSQGAVQRLQKAAERADDRGMRRLFSGIEAPFDLADARRAIESIVVSHRKRNKARGKFHRDTIYSQPLPGKDGRKGHRVRKELHKLKENQIKDIVDPRIRDVVGQAYQKLKTAGARTPAQAFSDPDNRPVLPHGDRIRRVRIFVSAKPDVIPGKDAPKSRRRCVDLQSNHHTVIMAKLNARGEEKTWVDEPVALLEAMDRVRDGKPLVCRDVPKGYRFMFSLAANDYVEMDRKDGDGRDVYRIRGISKGDIEVVQHHDGRTQTIRKAAKELDRVRGSTLQKRHARKVHVNYLGEVHDAGG

SEQ ID NO: 17 - protein, 1565 aa, Artificial Sequence
  Description of Artificial Sequence: Synthetic polypeptide
MTKILGLDIGTNSVGGALINLEEFGKKGNIEWLGSRVIPVDGDMLQKFESGAQVETKASSRTRIRMARRLKHRYKLRRTRIIQVFKLLKWVDESFPENFKEKKNNDPTFEFDINDYLPFTQASLEEAKNLLGITNKDGETKVPQDWIVYYLRKKALSEKISLQELARILYMMNQRRGFKSSRKDLEETSIIDYEAFKKYTNNNQYLDENGNTLETQFVVTTKIKSVEQKSDEKDSRGNYTFIITAESDRLQPWEEKRKKKPDWEGKEFKLLTTLKTRKSGKIEQLKPKAPSEDDWNLTMVALDNEIEESGKQVGEFFFDKLLNDKNYKIRQQVVKREKYQKELRAIWNKQLELNEDLNKLNEDPALLERIAKELYPTQTEFKGPKYKEITSNDLYHVFANDIIYYQRDLKSQKSLIDDCRYEKKKYFDKNLGKEVIQGYKVAPKSSPEFQEFRIWQDINNIKVIEKEKEIGGKLYPDINVTDEYVNNEVKARIFQLLDSKKEVSESQILKTIDKKLKPTAFKINLFANRDKLKGNETKSLFRSYLEQCGRENLLNDPDKFYKLWHILYSINGKDAEKGIRAALKNPKNEFDLSAEVIEELASLPEFSNQYAAYSSKAIHKLLPLMRSGDHWNHQSISQKIQDRINKIITSEEDEEIDNYTRDQITNYFKSQKNKDIWECELEDFKGLPVWLACYTVYGKHSEKDKKSWKSWKEIDVMKLVPNNSLRNPIVEQIVRETLHVVRDAWEKYGQPDEIHIEMSRELKNPKDERERISEIQNKNREEKERIKKLLFELKEGNPNSPIDINKFRLWKNNGGKEAQEKFDNLFNNKDEVSVSGDEIKKYRLWADQNHTSPYTGKPIPLSKLFTLEYEIEHIIPQSRMKNDSMSNLVISEAAVNDFKDRWLARPLIEKYGGTPIEHNGQTFTLLNQEEFEKHCNKTFQNQRGKLKNLLREEVPDDFVERQINDNRYITRKLGELLAPAAKADEGIVFTTGSITNELKDKWGFHTLWRELMKPRFERLEQILQKKLVVPDEKDTNKFHFNDPEPGNPVDIKRIDHRHHALDALIVAATTRAHIKYLNSLNSHKKREPYKYLANKGVRDFIQPWPDFTAEVKSQLKRLIVSHKVNCQYDPEHPEKSGVISKPKNRFKKWVNRDGVWKKEYQWQKDNENWWAIRKSMFKEPLGMIYLKEIKEVSLKKALEIQAERQKGIKDHTGRPRDYIYDKLARQEIRFLLEDKCGGDIKQAEKQSSTLKDSKSNPIKKVRVAFFKEYAASRVPVDNSFTYKKIKAIPYAEKIINRWEEWEQDGKNEKGQKFPNDITKWPIEFLLKKHLDEYKTSNGNPDPNTAFTGEGYEALTKKNGGQPIKKVTTYESKSAPIKFNGKILETDKGGNVFFVIAKDKHTGKHLDWYTPPLYSNEAEEGKERGIINRLINREPIAEDQEDLEYITLAPEDLVYVPEEDEDIRSIDWNGKDKQKVFERTYKMVSSTEKECHFIPHIVAYPILKTVELGTNDKSEKAWDGKVEYIPNKKGKLTRKDSGTMIKENCVKIKLDRLGNIIKVNGKPVNH

SEQ ID NO: 18 - protein, 1064 aa, Artificial Sequence
  Description of Artificial Sequence: Synthetic polypeptide
VSNARPSILPDDLILGLDIGTNSVGWALIHYAESEPRQLIALGSRVFEAGMDGSISHGKEESRNKKRRDARSLRRATWRRKRRKRRVYNLLHEAGLLPDADTNDPESINVALTRLDRELVSKFVSPGDHREAQLMPYLARRRAVEERVEPVVLGRALYHIAQRRGFRSNRRTAMREDEDLGQVKSAIASLHHKIVESEGEIQTLGGYFASLDPHEERIRTRWTGRDMYLEEFDKIVDRQIPYHDGLTSERVEALRAAIFDQRPLRSQNHLIGRCELERDQRRCSIALLEYQRFRLLQAVNNLRWLSDEGHERELSREERLRLVRELEIKPELAFGKIRTLLGLKRGTGRFNLELGGEKRLIGNRTNAQLRALFEARWETFTNDEQSSIVHDLMSIQNPIALQRRGQVRWGLDGEKSSYFANDLLLEDGYAPLSLRAIRKLLPRLEEGIPYSTARKEMYPESFQSSVVLDRLPPLAKTDLEARNPSIMRTLSEVRAVVNAIVRQYGRPGLVRIELARDLKQPKRRRQEISRQMREREGVREKAKKRLLDTEFGGSRASRADIEKLILADECDWTCPYTGRGFGMGDLFGSNPTIDVEHILPFSRCLDNSFLNKTLCDVRENRLVKRNRTPFEAYAGQRDRWEAILDRIKNFKSDPLTVRRKLERFLQEELSSARVDEFSERALSDTRYASRLVADFMGLLYGGRNDSDGKQRVQVSSGQATSILRREWGLNSLLGGEARKSRLDHRHHAVDAVVIALTGPREVKRLADAAKRAADQGSHRLFEEVPFPWTHFRTDVNEKIHCCVTSPRPSRRLRGPLHDESLYSRPLPWYDKKGRESLRPRIRKPIEQLTKGEVERIADPGVRDAVKTRAAELAKGQGGSGDLSKLFSDPSHAPFLRNRDGSTTPIRRVRITAKVKQATPIGEGVRQRHVAPGSNHHMAIVAILDEKGNEKRWEGHVVTMLEAVLRKGRGEPVIQRDWGKGQKFKFSLRSGDCIWNCDTGRIMHVKAVSAGVVEGLEVNDARTAVDVRRAGVVGGRYTASPERLRKDAFVRCVVDPLGKVIPSNE

SEQ ID NO: 19 - protein, 1024 aa, Artificial Sequence
  Description of Artificial Sequence: Synthetic polypeptide
VTYILGLDLGISSVGFAGIDHNGDNILFANAHVFDKAEVAKTGASLAEPRRNARLTRRRIERKARRKSRIKNLFDKYGLDVEAIDRPPSPDRQSVWDLRRVGLSKKLNSGQWARALFHLAKNRGFQSNRKDKADGVGTGKSDTDNGRMLSAISDLKKNLAESDHETIGSYLSTLDKKRNGDDDYSKTVHRDMIRDEVSLLFQRQRSFDNPHAGTELEQAFCKVAFYQRPLQSTIELIGNCSIFPDEKRAPKHAYSSEEFLAWSRLNNLRLLTPSGKKKELTTGQKEKAIELTKQYKKGVTFARLRRALDIDDQYRFNLCHYRNTMDGPSDWDTIRDKSEKQVLIQFPGYHAMRDQLSDLGADDIHFTELLANRDQYDDTIQILSFYEDEADILSRLSDLGHLPEVIEKLKYLDFSRTIDLSLKAVKQILPYMKKGYDYATARDMAGLKPKNTKSGNKKLLSPFDSTKNPVVDRCLAQSRKVVNAVIRRHGLPDYIHIELSRDLGRSKKERDKIDRRIEKNRRYKEDLRQHAAELLDREPSGEEFLKYRLWKEQDGICPYSGSYIEPDEWASPTAVQIDHILPFSRSYDNSYMNKVLCTASANQEKGNKTPYECWGQMDDLWPAIMAQADKLPKKKRDRILNKHFNEREQEFKTRHLNDTRYIARQLRQNISEQLDLGDGNRVRVRNGYITSFLRGIWGLQDKTRDNDRHHAIDAIIVACTTEGIMQQVTQWNKYDARRKDKEPYFPKPWDGFRSDVWDAYHAVFVSRLPDRSATGAMHKETVRSLRTDDDGNDVVVQRIPITDLSKAKLEDIVDKDTRNTRLYNTLKTRMEKHGYKADKAFAKPIYMPTNSDKQGPPIKRVRIVTNKQKDIVLPKRGGGVADRANMVRVDVFEKGGNFFLCPVYTDQIMRGELPMRLVKASKDESEWPEITDEYDFKFSLYKNDYVKIKKKSKGEIVELEGYYNGTDRATASISLRIHDNDQDVGKNGMIRGIGVYRLLSFEKYTVSYFGQLSRVNQGGRPGVA

SEQ ID NO: 20 - protein, 758 aa, Artificial Sequence
  Description of Artificial Sequence: Synthetic polypeptide
MSVRAIRARIACDRTVLDHLWRTHCVFHERLPIVLGWLFRMRRGECGETDAERLLYQRVGKFITGYSAQNADYLMNAVSLKGWKPATAKKYKIKTDDDNGQSVQISGESWADEAAALSAQGKLLFDKNVVSGGLPGCMRQMLNRESVAIISGHDELLSKWNTDHTKWLGEKAQWEAVPEHTLYLALRKKFESFEQAVGGKATKRRGRWHRYLDWLRANPDLAAWRGGPAIVDELSPAAQERIRKAKPWKKRSAEAEEFWKINPELASLDKLHGYYEREFVRRRKNKRNPDGFDHRPTFTMPDRIRHPRWFVFNAPQTNPSGYRHLRLPQGAKEIGAVQLQLITGGREGEGVYPTQWVDVTYRADPRLALFRRSQVSTTVNRGKAKGQTKIKEGYEFFDRHLSQWRSAEISGVKLIFRDIRLNDDGSLKSAIPYLVFACSIDDLPLTERAKKIEWSETGETTKTGKKRKSRTLPDGLIACAVDLGLRNVGFATLCVFEHGKSRVLRSRNIWLDDEGGGPDLGHIGQHKRQIKRLRRKRGKPVKGELSHVELQDHITHMGEDRFKKAARGIINFAWNVDGAVDEATGEPFPRADAIVLEKLEGFIPDAEKERGINRSLAAWNRGQLVTRLEEMAIDAGYKGRVFKVHPAGTSQVCSRCGALGRRYSITRDNAAHTPDIRFGWVEKLFACPCGYRANSDHNASVNLQRKFQMGDEAVKAFSSWRNQTEAQRQHALESLDASLRDGLRKMHGLPFPPLDNPF

SEQ ID NO: 21 - DNA, 3852 bp, Artificial Sequence
  Description of Artificial Sequence: Synthetic polynucleotide
atggaagaaaatagaagtcaaaaaaaatgcatatgggatgaattaacaaacgtttattcagtatcaaaaactctgcgttttgaattaaaaccattaggagaaaccttgaaaaatattaggaaaaaaggcttgatagaagaagataaaaaaagagacgaagattttttagaagtgaaaaaaataattgataaatatctaagttattttattgatagaaatttagatggttctaaaaacttaattgaagaacatcaattgaaagaaatacaagatatttatgaaaaactaaagaaaaatactactgatgaaaacttgaagaaagattatgcttctttacaaagtaaattaagaaaagaaatttttgctcaactgaaaacaaaaggccattataaagatttttttggaaagcaatttattaaaaaagttttattagattattataaagaagaagataacaaatatgatttattaaaaaaatttgaaaattggaatacttattttacaggattttatgaaaatagaaaaaatatttttaccgaaaaggatatttcaacttctttaacttatagaattgtaaatgataatttgccaaaatttttagataatattgcaaaatacaatgaactaaaaaatagtcttcctattcaagagatagaagaagagtttaaagattatttacaaggaatgcccttaaatgtattttttagtttaagtaattttaaaaattgcttgaatcagaagggaatagatacttttaatttattaattggcggaagaagtcctgatggtgagaaaaaaattaaaggattgaatgaatatatcaatgaactatctcaacatagtaatgatcctaaatctataaaaagacttaagatgatgcctttatttaagcagattttaggggagaataatactaattcatttcaatttgaaaaaatagaatatgatagagatctcataaatagaattgatgattttaataaaagattagaggaacaagatttatactctaatttatatgaaatttttaaagatttgaaagataatgatttgagaaagatatatattaaaaatggtaaagacataacaaatatatcacaacaattatttggggattgggacaaattatataaaggtctaagagaatatgcagaacaagatttattttcaagaaagaatgaaatagagaagtggctaaaaagaaaatatatttcaattcatgaattagaaaaagcaattgaaaaattaaaaattagtcaagaatttgataaaaaattatatgaaaattatttagaaaaaattaattataacgaaaacaatcctatttgtggttttctatctactttcaaacaaaaagagaaagatttgttagaagatataaaaacaaattattccaattatttggaaatatcaaaaaaagaatttggtgagggggatttgttaaaagaagattaccaaagagatgttgaaataattaaatcttatttggattctctaaaagagcttttacattatataaaaccactctatgttgatagcaaagacacagaagattcgaaacaacaagaagtatttgagcttgatgctaatttttatgaaacatttaatgaattatattttgaattaaaagaaataatccctctttataataaagtaagaaattatgtaactcaaaaaccttttagtacaaagaagtttaagttaaattttgaaaactcaacattactaaatggttgggataagaacaaagaaagagataatttttcagtaattttgagaaagaaaaatgaattaggaacttacgaatattttttaggtataatgtctagaggaaataataaaatctttgaaaacatagaagaaagtaatgaggatgattcttttgaaaaaatggattataaattacttcctggcccagataaaatgttgcctaaagtatttttttctgaaaaaaatattagttattataaaccctcagaagacatattggctattagaaatcattcctctcatactaaaaatggttctcctcaagaaggtttcatgaaaaaagaatttaataaagatgattgtcataaaatgatagatttttataaaaatgcattatctattcatcctgagtggtcaaattttgagtttaattttaaaaaaacctccttttatgaagatacttctgaatttttcaaagatatagctgatcaaggctaccaaatcaatttcagaaacatttcttcaaaagatattaatcaattagtagatgaaggaaaattatatttgttccaaatatataataaggatttttcaactaataaatctcaaaaaaatagaaatagtagaaaaaatcttcatactctatattgggaagaattattttctcctgaaaatcttagagatgttgtttataagttaaatggggaagctgaaatatttttcagagagaaatctattgagcctaaaacagaacaccccaaaaatcaagaaattaaaaataaggacccaattaatggaaaaaaatatagtaaattctcttatgatttaataaaagataaaagatatactgaagataaatttttatttcattgtcctatcacaatgaatttcaaagcaaaaggttcaaaatgggatataaataaaattgtcaatagtacaattaaagaaaattcaaaagaaattaatatattgagtattgatagaggtgagagacatcttgcatattggactttattaaattctaaaggagaaattgtagaccaagattcttttaatataattaaagaagagactattggaagaaaaacagattatcatgaaaaattatctgaaaaagaaggagatagggatgaagccagaaagaattggaagaagattgaaaatattaaagaattaaaagaagggtatttatctcaagtagttcataaacttgcaaaattagcagttgaagaaaatgcaattattgtttttgaggatttaaactatggttttaaacgaggaagatttaaaattgagaagcaagtatatcaaaaatttgagaaaatgttaattgaaaaattcaattatctaatgtttaaagatagagaaaaaaatgagattgcaggttcattaaacactctacaattaacgcctcaaataagttcagaaaaagaaaaaggtagacaaacaggagtaatattttatactgatcctaattatacatcaaagatagatcctaaaacaggttttattaatttattatatcccaaatatgaatcagttgagaaatcaaagaattttttcaaaaaatttgaatcaattaaatataatggagaatattttgaatttacttttaattattctaatttttataatgatttaaatttaacaaaaaaagagtggacaatttgttcatatggcgataggattttctcttttagaaatcctgaaaaaaataatcaatttgacactaaaacaatttatccaacagatgaactgaaatcattgtttgataaatattatattgaatatgaaagtcaaaaaaatattttaaatgaaataaccaaacaaagttcaagtgatttttacaaatcattaatgtttattttaagtaagatattacaattaagaaattctataccaaattccgaagaagattttatcttgtcatgtataaaagataaaaaaggtaatttctttgattcaagaaatgctaataaaaacacagaacctgcaaatgcagattcaaacggagcttataatattggaataaaaggtttaatgataattgagagaattaaaaattgtccagaagataaaaaacctaatttaacaattaagagggatgaatttgtgaattatgtaatagggaggaatacatag

SEQ ID NO: 22 - DNA, 3852 bp, Artificial Sequence
  Description of Artificial Sequence: Synthetic polynucleotide
atggaggaaaaccgtagccagaagaaatgcatctgggacgagctgaccaacgtgtacagcgttagcaaaaccctgcgtttcgagctgaagccgctgggtgaaaccctgaaaaacattcgtaagaaaggcctgatcgaggaagataagaaacgtgacgaagacttcctggaagtgaagaagatcattgacaaatacctgagctatttcattgatcgtaacctggacggcagcaagaacctgatcgaggaacaccagctgaaagagatccaagatatttacgaaaagctgaagaaaaacaccaccgatgagaacctgaagaaagactatgcgagcctgcagagcaaactgcgtaaggaaatctttgcgcaactgaagaccaaaggtcactacaaggatttctttggcaaacagttcattaagaaagttctgctggactactataaggaagaggacaacaaatatgacctgctgaagaaatttgaaaactggaacacctacttcaccggtttctacgagaaccgtaagaacatcttcaccgaaaaggacatcagcaccagcctgacctaccgtattgtgaacgataacctgccgaaatttctggacaacatcgcgaagtataacgagctgaaaaacagcctgccgatccaggaaattgaggaagagttcaaggattacctgcaaggcatgccgctgaacgttttctttagcctgagcaacttcaaaaactgcctgaaccagaagggcattgatacctttaacctgctgatcggtggccgtagcccggacggcgagaagaaaattaaaggcctgaacgaatacatcaacgagctgagccaacacagcaacgacccgaaaagcattaagcgtctgaaaatgatgccgctgttcaaacagatcctgggcgaaaacaacaccaacagcttccaatttgaaaagatcgagtacgaccgtgatctgatcaaccgtattgacgattttaacaaacgtctggaagagcaggatctgtacagcaacctgtatgagatcttcaaggacctgaaagacaacgatctgcgtaagatctacatcaagaacggcaaggacatcaccaacattagccagcaactgtttggtgactgggataagctgtacaaaggcctgcgtgaatatgcggagcaagacctgttcagccgtaagaacgaaatcgagaaatggctgaagcgtaaatacatcagcattcacgaactggagaaagcgatcgagaagctgaaaattagccaggaatttgacaagaaactgtacgaaaactatctggagaagattaactataacgagaacaacccgatctgcggcttcctgagcacctttaagcaaaaagagaaggatctgctggaagacattaaaaccaactacagcaactacctggagatcagcaagaaggagttcggcgagggcgacctgctgaaagaggactaccagcgtgacgtggaaatcattaagagctatctggatagcctgaaagagctgctgcactacatcaagccgctgtatgtggacagcaaagataccgaagacagcaagcagcaagaagtttttgagctggacgcgaacttctacgaaacctttaacgagctgtatttcgaactgaaagagatcattccgctgtacaacaaagtgcgtaactatgttacccaaaaaccgtttagcaccaagaaattcaagctgaactttgagaacagcaccctgctgaacggttgggataaaaacaaggaacgtgacaacttcagcgtgatcctgcgtaagaaaaacgagctgggcacctacgaatatttcctgggtattatgagccgtggcaacaacaagatctttgagaacattgaagagagcaacgaggacgatagcttcgaaaagatggattacaaactgctgccgggtccggacaagatgctgccgaaagttttctttagcgagaaaaacatcagctactataagccgagcgaagacatcctggcgattcgtaaccacagcagccacaccaaaaacggtagcccgcaggaaggtttcatgaagaaagaatttaacaaggacgattgccacaaaatgattgatttctacaagaacgcgctgagcatccacccggagtggagcaacttcgaatttaacttcaagaaaaccagcttttacgaagataccagcgagttctttaaagacatcgcggaccagggttatcaaatcaacttccgtaacattagcagcaaggacatcaaccagctggttgacgagggcaaactgtacctgttccaaatctataacaaggactttagcaccaacaagagccagaaaaaccgtaacagccgtaaaaacctgcacaccctgtactgggaagagctgttcagcccggaaaacctgcgtgatgtggtttataagctgaacggcgaagcggagattttctttcgtgaaaagagcatcgagccgaaaaccgaacacccgaagaaccaagagattaaaaacaaggacccgatcaacggtaagaaatacagcaagttcagctatgatctgatcaaagacaagcgttacaccgaagacaagtttctgttccactgcccgattaccatgaacttcaaagcgaagggtagcaaatgggacatcaacaagattgtgaacagcaccattaaggagaacagcaaagaaatcaacattctgagcatcgaccgtggtgagcgtcacctggcgtactggaccctgctgaacagcaaaggcgaaatcgttgaccaggatagcttcaacatcattaaagaggaaaccattggtcgtaagaccgattatcacgagaagctgagcgaaaaagagggcgaccgtgatgaggcgcgtaagaactggaagaaaatcgaaaacatcaaggaactgaaagagggctacctgagccaagtggttcacaagctggcgaaactggcggtggaagagaacgcgatcattgtttttgaggacctgaactatggtttcaaacgtggccgttttaagatcgaaaagcaggtttaccaaaagttcgagaaaatgctgatcgaaaagttcaactatctgatgtttaaggatcgtgagaagaacgagattgcgggtagcctgaacaccctgcagctgaccccgcaaatcagcagcgaaaaagagaagggtcgtcagaccggcgtgatcttctacaccgatccgaactataccagcaagattgacccgaaaaccggtttcatcaacctgctgtacccgaaatatgaaagcgttgagaaaagcaagaacttctttaagaagtttgagagcatcaagtacaacggcgaatattttgagttcacctttaactacagcaacttctataacgatctgaacctgaccaagaaagaatggaccatttgcagctacggtgaccgtatcttcagctttcgtaacccggagaaaaacaaccagtttgataccaagaccatctacccgaccgatgaactgaaaagcctgttcgacaagtactatattgaatatgagagccagaaaaacatcctgaacgagattaccaagcaaagcagcagcgacttctacaaaagcctgatgtttatcctgagcaagattctgcaactgcgtaacagcatcccgaacagcgaagaggatttcatcctgagctgcatcaaggataagaaaggtaacttctttgacagccgtaacgcgaacaagaacaccgagccggcgaacgcggacagcaacggtgcgtacaacatcggtattaaaggcctgatgatcattgagcgtatcaagaactgcccggaagataagaaaccgaacctgaccattaaacgtgacgagttcgtgaactatgttatcggtcgtaacacctag

SEQ ID NO: 23 - DNA, 3414 bp, Artificial Sequence
  Description of Artificial Sequence: Synthetic polynucleotide
atgataaatattgacgaattaaaaaatttatataaagttcaaaaaacaattacttttgaattaaaaaataaatgggaaaataagaatgatgaaaatgatagagttgagtttttaaagactcaagaatgggtggaatctttattcaaagttgatgaggagaattttgatgaaaaggagtcaattccgaacttgttagatttcggccaaaagattgcgagtcttttttataagttgagtgaagatatcgctaataatcaaattgatacacgggttttaaaagtgagcaagtttttgttggaggagatcgatagaaatcaatatcatgagaaaaaaaataaaccaacaaaggttaaggagatgaatccaaatacaaataagagttatattaaggagtataagttatcagatcaaaatacattgtatgttctgttgaagataatggaagatgaagggcggggtttacaaaaatttttatatgataaggcagacagattaaatttatataatcagaaggtaagaagagatttcgctttaaaagaaagtaacgaacagcagaagttttcgggtaacgctaattattacggaaacataaaattgttgattgattcattggaagacgctgttcgtattattggttatttcacgtttgatgatcaagcagaaaatgctcaaataaatgaattcaagagcgttaagcaggaaatgaataacaatgaagcttcgtatcaggctttgaaagattttgctattgataacgcaaaaaaagaaattgaacttacaactctaaatcatagggctgttaacaaggatccaaaaaagatacaagaacagattgaagaagtggaaaattttgaagaagatataaatcaattgaagcaccaaatttctgcgcttaatgataaaaaatttgatgtagtgtcaagattaaagcatgcattaattaaaatgttaccggagttgaatttgttagatgctgaaagcgagcaaggtagagaggttcagcaaatatatcaagataaaaagaatggtttggaattagacgattttaagttcaatttgcttaaacatcatcaatggcagaaaaccatttttaaatacattaaattagagggtttggttttacctgatttatatgccgaaaacaaacaagataagattaaagtgtatattgaaaattatcgacaaagcggagaaaggataagtaaaaaggcacgcgaggagttgggcaagatcgataaaagagaggaatttaatggtaatgatgaactaaagaaagcgtggtacgaatacaaagatttttgcagagacaagcgtaataaatccgtggaattgggcaataagaaatcactgtacaatgccatcaagcgtgaggttttaaggcagaaaatgtgtaatcattttgccgtattggtgagtgatggggaagatacatcgccttattattatttgatattaattcccaatgaaaacagtgatgaaatgaacaggacattcaaagagcttaaagcatccgaaggaaattggaagatgctcgattataacagattaacttttaaagctttggaaaaattggcattattgcgcagctctacatttgaaattgcagaccaagaactacaagaagaagctaaaaaaatttgggaagaatataaagaaaaggcgtataaagattttaagaataaaaaattattacaagggctatccggtcgccaaagagaagaaaaaaaacaagaattgcaaaaagaaagtttaaatcgagttataaattatttaattcgttgcattcagtcgttgccggatagcggtaaatacaattttaattttaaagaaccgcatcaatatcagagcttggaagagtttgcggaagaaattgatagacagggttatcattgcgcttggaagaatgtaagcaaagacaagcttatggagctggaggcgatggaaaaaattaaagtatttaaattgcataataaggattttagaaaagttaaacttaacgattcgaaacacaatccgaatctttttactttatattggcttgacgcgatgaatttggataaagtcaatgttcgtttattgcccgaggtggatttatataaaagagccaaagaaacgcaactaaaattattcgaaagagatgtaaagtgcaatattaataatcaaaaaataaaatcaattaaagaaaaaaatagattatttcaagataaactttacgcttcattcaagctggaattttatccagaaaacgaaggtttgggttttgaacaagtcaatgataaagtgaataatttttgcggaagtgatacagcgtattatttgggtttggataggggtgagaaagaattggttacgttttgcttggttgattctgatgggcggttggttaagaacggagattggacgaagtttaaagaggttaactatgcggataaattaaagcaattttattattcaaaaggtgaaatagaatctactcaacaacaacttttggaagctcgagacaatattaaacaagctactaacacggaggataaagaatcgatgaaattaaactataaaaaattagagttgaaactaaaacaacagaatttgttagcgcaggagtttattaaaaaagcttattgcggttatttgatagattcaataaatgaaatattacgggaatatccaaatacgtatcttgtattagaggatttggatatagcaggtaaagctgaccccgaaagcggcatgaccaataaagaacaaaatttaaataaaacaatgggtgccagcgtttatcaagctattgaaaatgccatagtaaataagtttaaataccgtactgttaaattatccgatatcaaaggtttgcaaactgtaccgaatgtagtgaaggtggaagatttgcgcgaagttaaggaagtggaagatggtgagcataaatttggtttgataagatccgtgaaatcaaaggatcaaattggcaatattctgtttgtggatgaaggagaaacatctaatacttgcccgaattgcggatttaacagcgattggtttaagcgggatgttgattttgatttggagattgtggctactgtaaacggtcagaaaaatgcggttatagaacaaaacgacaaaaagtactgttttcccggtgaaatttataagttagaaataattaataaagaatacgaaacaaataaacggaatttagccatgatttttaaaccgcgcgcaaaagcttgtagaaaatttataaataataatttggataagaatgactatttttattgcccgtattgcgctttttctagcaagaactgcaataatccaaaattgcaaaacggtgattttgtggtatattcgggtgatgatgtggcggcatacaatgtagcgatcagaggtattaaccttttaaacaatataaaatag

SEQ ID NO: 24 - DNA, 3414 bp, Artificial Sequence
  Description of Artificial Sequence: Synthetic polynucleotide
atgattaacatcgacgaactgaaaaacctgtataaagtgcagaagaccatcacctttgaactgaaaaacaagtgggaaaataagaatgacgagaacgatcgtgtggagttcctgaagacccaggagtgggtggaaagcctgttcaaagttgatgaggaaaactttgacgagaaggaaagcattccgaacctgctggacttcggtcaaaagatcgcgagcctgttttataaactgagcgaagatattgcgaacaaccagatcgacacccgtgtgctgaaggttagcaaatttctgctggaggaaattgatcgtaaccaataccacgagaagaaaaacaaaccgaccaaggtgaaagaaatgaacccgaacaccaacaagagctatattaaggagtacaaactgagcgatcagaacaccctgtacgttctgctgaagatcatggaggacgaaggtcgtggcctgcaaaaattcctgtatgataaggcggaccgtctgaacctgtacaaccagaaagttcgtcgtgacttcgcgctgaaggagagcaacgaacagcaaaaatttagcggtaacgcgaactactatggcaacattaaactgctgatcgatagcctggaggacgcggtgcgtatcattggttatttcacctttgacgatcaagcggagaacgcgcagatcaacgagttcaagagcgttaaacaagagatgaacaacaacgaagcgagctaccaggcgctgaaagattttgcgattgacaacgcgaagaaagagatcgaactgaccaccctgaaccaccgtgcggtgaacaaggacccgaagaagatccaagagcagatcgaggaagttgaaaacttcgaggaagacattaaccaactgaaacaccagatcagcgcgctgaacgataagaaatttgacgtggttagccgtctgaaacacgcgctgattaagatgctgccggagctgaacctgctggatgcggagagcgaacaaggtcgtgaagtgcagcaaatctaccaggacaagaaaaacggcctggagctggacgatttcaaatttaacctgctgaagcaccaccaatggcagaaaaccattttcaagtatatcaaactggagggtctggtgctgccggatctgtacgcggaaaacaagcaagacaagatcaaggtttacatcgagaactaccgtcagagcggcgaacgtattagcaagaaagcgcgtgaggaactgggcaagatcgataaacgtgaggagttcaacggcaacgacgagctgaagaaagcgtggtatgaatacaaggatttttgccgtgacaagcgtaacaaaagcgtggaactgggtaacaagaaaagcctgtacaacgcgatcaagcgtgaggttctgcgtcagaaaatgtgcaaccacttcgcggtgctggttagcgatggcgaggacaccagcccgtactattacctgatcctgattccgaacgagaacagcgatgaaatgaaccgtacctttaaggagctgaaagcgagcgagggtaactggaaaatgctggactacaaccgtctgaccttcaaggcgctggaaaaactggcgctgctgcgtagcagcacctttgagattgcggatcaagaactgcaggaagaggcgaagaagatctgggaggaatataaggagaaagcgtacaaggacttcaagaacaagaaactgctgcaaggtctgagcggccgtcagcgtgaggaaaagaaacaagagctgcagaaggaaagcctgaaccgtgtgatcaactatctgatccgttgcattcaaagcctgccggacagcggtaaatataacttcaactttaaggaaccgcaccaataccagagcctggaggagttcgcggaggaaattgatcgtcagggctaccactgcgcgtggaaaaacgttagcaaggacaaactgatggagctggaagcgatggagaagatcaaagtgttcaagctgcacaacaaagattttcgtaaggttaaactgaacgacagcaaacacaacccgaacctgtttaccctgtattggctggatgcgatgaacctggacaaggtgaacgttcgtctgctgccggaagtggatctgtacaagcgtgcgaaagaaacccagctgaagctgttcgaacgtgacgttaaatgcaacatcaacaaccaaaagatcaaaagcattaaggagaaaaaccgtctgtttcaggataaactgtatgcgagcttcaagctggagttttacccggagaacgaaggtctgggcttcgaacaggtgaacgacaaggttaacaacttttgcggtagcgataccgcgtattacctgggtctggaccgtggcgagaaagaactggtgaccttctgcctggttgacagcgatggtcgtctggtgaagaacggcgattggaccaagttcaaagaagttaactatgcggacaagctgaaacaattttattacagcaaaggcgagattgaaagcacccagcaacagctgctggaggcgcgtgataacatcaagcaggcgaccaacaccgaggacaaggaaagcatgaaactgaactacaagaaactggagctgaagctgaaacaacagaacctgctggcgcaggaatttattaagaaagcgtattgcggttacctgatcgatagcattaacgagatcctgcgtgaatatccgaacacctacctggtgctggaagacctggatatcgcgggtaaagcggacccggagagcggcatgaccaacaaagaacaaaacctgaacaagaccatgggtgcgagcgtttatcaggcgattgagaacgcgatcgtgaacaagttcaaataccgtaccgttaaactgagcgacattaagggcctgcaaaccgtgccgaacgtggttaaggttgaggatctgcgtgaagtgaaagaggttgaagacggcgagcacaagttcggcctgatccgtagcgtgaagagcaaagatcagattggtaacatcctgtttgttgacgagggcgaaaccagcaacacctgcccgaactgcggcttcaacagcgattggtttaaacgtgacgtggatttcgacctggaaattgtggcgaccgttaacggtcaaaagaacgcggttatcgagcagaacgacaagaaatattgctttccgggcgagatctacaaactggaaatcattaacaaggagtacgaaaccaacaaacgtaacctggcgatgattttcaagccgcgtgcgaaagcgtgccgtaagtttatcaacaacaacctggataagaacgactatttctactgcccgtattgcgcgtttagcagcaagaactgcaacaacccgaaactgcagaacggtgacttcgtggtttacagcggcgacgatgtggcggcgtataatgtggcgatccgtggtatcaatctgctgaataatatcaagtag

SEQ ID NO: 25 - DNA, 3780 bp, Artificial Sequence
  Description of Artificial Sequence: Synthetic polynucleotide
atgcatctatctcaaacatttacaaacaaatatcaggtatcaaaaacattaaggtttgaacttaggccacaaggccaaaccaaggaaaaatttgaaagatggattgctgaactaagaacagaaaacccaagtgctgataatttaatcgcagaagatgagcaaagagcagtagattataaagaagtaaaaagtatcatagatcgttttcatagaaaagtgattgaagaaagtttggagggcttgaagttgaaaggactatcagaatatgaggaactctattttaagcgtgaaaaagaagatatcgaccttaaggagatagaaaatctgcaaatacaaatgcgaaagcaaattagagaggcatttgttgaacaccctgtttttaaagatttattcaaaaaagaattgattcaagttcatttaaaagaatggcttacggatcaacaagagattgatttggttgccaagtttgaaaaattcaccacctactttggtggttttcatgagaatcgacagaatgtctatagtccggatgcaaaagctaccgcagtgggctacagaatgattcatgaaaacttgccgaagtttttagacaatcgaagaatttttaataaaatcataaaagcacatgaagagctagatttctcatcaattgattcagagttagaagagcttttacaaggaactactgttgaggaagttttttcgctagaattttataacgaaacactgacgcaaaccggaatcgatatttataatcatgtattgggaggctattcttctgaaacaggacaaaagattcagggagtgaatgagaaaatcaatttgtaccgacagaagaatgggttaaaagccagagagttgcccaaccttaagccattattcaaacaaatattgagtgaaagtcaaaccgcttcttttgtcatagagcaaatagaaagtgaatcggatttattagacaggctagacaattttcacaccctaataacaagtttcgaatttcaaggaagaaatcaagtaaatgtaatgaccgagctcaagcatatgttagcagcgctagattcatatgaacatgagcaagtatattttaaaaatggcccaagtcttactcaattatcacaaaagatgtttgggcaatggggcgtgattcataaggcactggaatattattatgagcaagagcaaaatcctttacaaggtaagaaactgactaaaaaatatgagaatgataaagagaaatggttaaaaaataaacagttcaatttgagccttttgcagaaggcaatagatgtctatgtgccaacgatcgataccatagaacctgtcagtatagtagaaacactttccacgttagaagacaaagaaggtgcagatttaggtacggaagtggataatgcttacgagaaagtagctgaattaatagagcaaaagacattgagtgaaagctacgcacaaaaaaagaaggagaagcaagtcattaaagaatatctcgatggtttaatgagtcttttacatagtgtaaagcctttttatacgaccgaggttgatatagaaaaagatgccggattttacgggttatttgaaccgctgtatgagcaactaaacctagtaattcctatttataatttggtgagaaattacctcacacaaaaaccttattcaactgaaaaatttaaactgaattttgaaaataatactcttttggatggttgggatcagaataaagagaaggcaaatacatgcgtattattaaggaaagagggtaattattatttggcggttatgcacaaaaatcacaacacggtatttgaagagctgccccaaaatgaaaatgcgacttatgaaaaagtaatttataaacttttgcctggagccaataaaatgttacccaaggttttcttttcaaaaaagaatatagactactataaacccaaagaagaacttttagaaaaatataagctaggcactcataaaaagggaagtaatttcaatctcaaagactgtcatgcgctaattgattttttcaaggactccatttccaaacatcctgattgggctcaattcaattttgagttttcacaaacaaaaacctatgaagatttaagccatttttacagagaagtagagcatcagggatacaaaatcaattatgcaaaggttgatgtttcttacatcaatcaattggtagatgacgggagaatttttctatttcaaatttataacaaagacttttctccatacagcaagggcaaacccaatttgcataccatgtattggagagctgttttcgatgaaaaaaacttagcagatacggtatataaactgaacggaaaagccgagatattttttagagaaaagtcgctcaactactctaaagaaatcatggaaaaagggcatcatcgagacgaattgaaggataaattttcttaccctattatcaaggataaacgatttgccttggataagtttcagtttcatgtcccattaacaatgaactttaaggcgggaagcaatccaaatttaaacgaccgtgcattggatttcttaaaagataatcccgatataaaaatcattggcttggacagaggagagcgacacctactctacttgagcctgattgatcaaaaaggaaatataattgagcaatacacattgaatgagattgtttcaaaacacaaagacaaaacctttaaaaaagactatcacgagctattagataagaaagaaaaggggcgtgatgatgctcgaaaaaattgggatgttatcgaaacgattaaggaattaaaagagggatacctttctcaggtagttcacaaaattgctcaaatgatgattgagcacaactcaattgttgtattagaggatttaaacgctggctttaaaagaggaaggcataaggtagaaaagcaagtttatcagaagtttgagaaaatgctcattgataaattgaattatttggtttttaaagaccatgataaggaaaaacctggaggtttactgaacgctcttcaactcacaaataaattcgaaagttttcaaaaattaggtaaacaaagcggtcttcttttttatgtacctgctgctttaacaagtaaaattgatcctgctacaggttttacgaatttcttaagaccaaagcatgaaagcatccccaaatcccaatctttcatcgcaggctttacccgaattcattttaattcggagaaagaatatttcgagtttaaattcgatttgaaaaacataccgaatacacgctttcctgatgatacaaaaactgaatggacggtatgtacaacaaatgtgcctcgttattggtggaacaagagtttgaatgaaggtaaagggggacaagaaaaggtcttagtaacacaaaggctgcaagatttattggcaaggtatgatttaggctatgcaactggtgaaaacttaaaggaagatattttaacaattgaagatgcctctttctacaaggagttcttatggttgttgaatgtaactgtttcattgcggcacaataatggtaagcatggagaactagaagaagatgcgatcatttcacccgtagcgaatgcacaaggcgaatttttcaattcgagtgaggcaaagtcttcagcccctaaagatgctgatgccaatggagcttatcatattgcacttaaaggactttgggctttacgaacaattaatgcacacgacaagaaagaatggagaggtataaagttagccatatctaacaaagaatggttgcagtttgtgcagcaaaagccttttcttaaaccatag

SEQ ID NO: 26 - DNA, 3780 bp, Artificial Sequence
  Description of Artificial Sequence: Synthetic polynucleotide
atgcacctgagccagaccttcaccaacaagtaccaagtgagcaaaaccctgcgttttgagctgcgtccgcagggtcaaaccaaagagaagttcgaacgttggatcgcggagctgcgtaccgaaaacccgagcgcggataacctgattgcggaggacgaacagcgtgcggtggattataaggaagttaaaagcatcattgaccgttttcaccgtaaggttatcgaggaaagcctggagggtctgaaactgaagggcctgagcgaatatgaggaactgtacttcaagcgtgagaaagaagacatcgatctgaaggagattgaaaacctgcagatccaaatgcgtaaacagatccgtgaggcgttcgtggaacacccggttttcaaggacctgtttaagaaagagctgatccaagtgcacctgaaagagtggctgaccgatcagcaagaaattgacctggttgcgaagttcgagaaatttaccacctacttcggtggctttcacgaaaaccgtcagaacgtgtatagcccggatgcgaaggcgaccgcggttggttatcgtatgatccacgagaacctgccgaaattcctggacaaccgtcgtatcttcaacaagatcatcaaggcgcacgaggaactggatttcagcagcatcgacagcgaactggaggaactgctgcagggcaccaccgtggaggaagttttcagcctggagttttacaacgaaaccctgacccaaaccggcatcgacatttacaaccacgtgctgggtggctatagcagcgaaaccggtcagaagatccaaggcgttaacgaaaaaattaacctgtatcgtcagaagaacggcctgaaagcgcgtgagctgccgaacctgaagccgctgtttaaacagatcctgagcgagagccaaaccgcgagcttcgtgatcgaacaaattgagagcgaaagcgacctgctggatcgtctggacaacttccacaccctgattaccagcttcgagtttcagggtcgtaaccaagtgaacgttatgaccgaactgaagcacatgctggcggcgctggatagctatgagcacgaacaggtgtactttaaaaacggcccgagcctgacccagctgagccaaaagatgttcggtcaatggggcgttatccacaaagcgctggagtactattacgagcaggaacaaaacccgctgcagggtaagaaactgaccaagaaatacgagaacgacaaagaaaagtggctgaaaaacaagcagttcaacctgagcctgctgcaaaaggcgatcgatgtgtatgttccgaccatcgacaccattgagccggtgagcattgttgaaaccctgagcaccctggaggataaagaaggtgctgacctgggcaccgaggtggataacgcgtacgaaaaggttgcggagctgatcgaacagaaaaccctgagcgaaagctacgcgcagaagaaaaaggagaagcaagtgatcaaggaatatctggacggtctgatgagcctgctgcacagcgtgaagccgttctataccaccgaggttgacatcgaaaaagacgcgggtttctacggcctgtttgagccgctgtatgaacagctgaacctggtgatcccgatttataacctggttcgtaactacctgacccaaaaaccgtatagcaccgagaaattcaagctgaactttgaaaacaacaccctgctggatggttgggaccagaacaaagagaaggcgaacacctgcgttctgctgcgtaaggaaggcaactattacctggcggtgatgcacaaaaaccacaacaccgttttcgaggaactgccgcaaaacgagaacgcgacctatgaaaaggtgatctacaaactgctgccgggtgcgaacaagatgctgccgaaagttttctttagcaaaaagaacatcgattactacaagccgaaagaggagctgctggagaaatacaagctgggcacccacaaaaagggcagcaactttaacctgaaggactgccacgcgctgatcgatttctttaaggacagcattagcaaacacccggattgggcgcagttcaactttgagttcagccaaaccaaaacctacgaagacctgagccacttctatcgtgaggtggaacaccagggctataagatcaactacgcgaaagtggatgttagctacattaaccagctggttgacgatggtcgtatttttctgttccaaatctacaacaaggactttagcccgtatagcaaaggcaagccgaacctgcacaccatgtactggcgtgcggtgttcgacgagaagaacctggcggataccgtttataagctgaacggtaaagcggagatcttctttcgtgagaagagcctgaactacagcaaggagattatggaaaaaggccaccaccgtgatgaactgaaagacaagttcagctatccgatcattaaagacaagcgttttgcgctggataagtttcagttccacgttccgctgaccatgaactttaaagcgggtagcaacccgaacctgaacgatcgtgcgctggacttcctgaaggataacccggacatcaaaatcattggtctggatcgtggcgagcgtcacctgctgtacctgagcctgatcgaccagaaaggcaacatcattgagcaatataccctgaacgaaattgtgagcaaacacaaggacaaaacctttaaaaaggattaccacgagctgctggacaaaaaggaaaagggtcgtgacgatgcgcgtaaaaactgggacgttatcgaaaccattaaggagctgaaagaaggctatctgagccaggtggttcacaagattgcgcaaatgatgatcgagcacaacagcattgtggttctggaagatctgaacgcgggtttcaaacgtggccgtcataaggtggagaagcaggtttaccaaaagttcgaaaagatgctgatcgacaagctgaactatctggtgttcaaagaccacgataaggagaaaccgggtggcctgctgaacgcgctgcagctgaccaacaagttcgagagcttccagaagctgggtaaacaaagcggcctgctgttctacgttccggcggcgctgaccagcaaaatcgatccggcgaccggtttcaccaactttctgcgtccgaagcacgagagcattccgaaaagccagagcttcatcgcgggctttacccgtattcactttaacagcgagaaggaatactttgagttcaagtttgacctgaaaaacatcccgaacacccgtttcccggacgataccaagaccgaatggaccgtgtgcaccaccaacgttccgcgttattggtggaacaaaagcctgaacgagggcaagggtggccaggaaaaagtgctggttacccagcgtctgcaagatctgctggcgcgttatgacctgggttacgcgaccggcgagaacctgaaagaggacatcctgaccattgaggacgcgagcttctacaaagaatttctgtggctgctgaacgtgaccgttagcctgcgtcacaacaacggcaagcacggcgagctggaggaagatgcgatcattagcccggtggcgaacgcgcagggcgagttctttaacagcagcgaagcgaagagcagcgcgccgaaagacgcggatgcgaacggtgcgtaccacatcgcgctgaaaggcctgtgggcgctgcgtaccattaacgcgcacgacaaaaaggagtggcgtggcatcaagctggcgattagcaacaaagaatggctgcaattcgttcagcaaaagccgtttctgaaaccgtag

SEQ ID NO: 27 - DNA, 4011 bp, Artificial Sequence
  Description of Artificial Sequence: Synthetic polynucleotide
atgaaacaagaaaagaagacagaaaaatccgtgttctcggattttacaaataaatacgcactttcgaagacgttgcgatttgagttgaagccggtgggagagacgcttgaaaatatgaaagatgcttttggatatgacaaaaaaatgcaaacttttttgaaagatcaagaaatcgaagatgcgtatcaaaacctcaagcccattctcgatagaattcacgaagaattcattacacaaagccttgaatcagaacaagcaaaacaaattccatttcatatatatgaaaaatcttatagaaaaaagagcgaaattacactcaagcagtttgaaacggttgagaaaaaaatacgagagtattttgacgaagcgtataaacaaacagctcaagtgtggaagcagaatgctccaaaagacaaaaaagggaagggggtatttacaaaagattctcacaagctccttactgaggtgggagtgcttgaatatattcgtcaaaatacggagaaattttcagacattcttccgaaaagtgaaatagagcaacatctcaatgtttttagtggattttttacctatttccaaggatttagtcaaaatagagaaaattactatacaacaaaggatgaaaaagcaacggcggtagcaacaagagttgtcagtgaaaatcttccgaaattttgtgacaacatcctaacctttgagaacaaaaaagaagcgtacctcgctctgtatcaatctttggctgagaaggggaaaacacttcagataaaagatgggtcatcaggaaaaatgaaatctcttgaaggggtggatgaagcaatgttttcaatacatcatttcaatgaatgtctttcacaaagagagattgagaaatataatgaggcaatagccaatgctaattatcttataaacctctataatcaattacaagatgacaagaagaataaacttaagcttttcaaaactctctacaaacaaatagggtgtggggataaggaaacgtttatcgagaagataactcactacacagaagaagaggcacaaaaagctcgaaaagaaaaaaaggaaaaagcaatatcacttgaacaggaattaaaagagttttctagtttgggaagtaaatattttttcggtatatcagaaaatgagtttattagaacagtagaagatttcagaaagtatctcttagaagaaaaagaagattatgcgggagtctattggtcaaaacaggcgataaacaatatatcggggaaatatttttctaattggcatgcacttaaagatattctcaaagaaaaaaaggtttttagcacgagcgcttccaaagatgaatcggtgagcatcccggagataattgaactcaagcaactttttgaggttcttgatggaattgagaagtgggaagtacctgataattttttcaaaaagacgcttacagaggaggtaagtaaagatcatagagatttccagaaaaatgcaaaaagaaaagagatcattaaatcatcccaaaaaccatcagaagcacttctgaggatgatgtttgatgatatggttgatcttcgagagaaatttctttccaaaaaagaagacattttggaaaatacaaactatactactcaagaaagaaaggatgatataaaagaatggatggattcgggattgagaattattcaaattctcaaatacttttctgtccaagaaaagaagataaaagggacaccatttgacgccaaaatcaaagaagggcttgacactctccttctctccaatgaagtggactggtttacaagatatgatcgcgtacgaagttttctcactaaaaaaccgcaagatgatgcgaaagaaaataaattgaagttgaattttgagaatagcacgcttgctggtgggtgggatgtgaacaaagaaagtgataactcttgcatcattttgaaagaggaagaaaaaacattcttagccgtgatagcaaaatcaaaagggaaagagaaaaataatgctttgtttcgaaaaacagaacaaaatccacttttttctattgagaatgcggagacaatgaaaaaaatggagtataagcttctccccggtccaaataaaatgttgccgaagtgtctttttcccaagtcgaatcctaagaaatatggagcaactgaaactgttcttgatgtgtataaaaaaggaagttttaagaagaacgaagaaaatttctccaaaaaagatttatacactgtaattgatttttacaaggaggctttgaagagatatgaaggatggaattgttttgaatttcattttaaaaagacgagtgaatacaatgatattggtgaattttatttagatgttgaaaagaaaggatacactttggattttgtagatattaacagaaatgtccttggacagtatgttgaagatggaagggtgtatcttttcgaaattcgaaataaagactggaatacactacctgatggatcgaagaaaagcggaaatacaaatctccatactatgtactggaaagcattgtttcaagatagagaaaatcgaccaaaactcaatggagaggctgagattttttatagaaaagccttatcaaaagatgaaataaagaagaaaaaagataaacatgaaaaggaagttattgaaaattatcgattttccaaagaaaaatttctttttcatgtgccaataacgctcaacttttgtctcaaggattataaaatcaacgacgatataaacgaaaagctccttgaaaatgagaatgtatgctttttggggattgataggggagaaaagcaccttgcctattattcgatagttgataacgagggaaatattttggaacaagatacactcaatacgataaacggaaaagactacaatactcttctcgaagaacgatccgaagagatggataccgctcgaaaaagttggcagactattggaacgattaaagaactcaaagacggctatatttctcaagttatccgaaaaattgtcgatctctctcttcgatacaatgcatttattgtcttagaagatctcaatgttgggttcaaacaaggtcgccaaaaaatcgaaaaatccgtttaccaaaaactcgagcttgctttggcgaaaaaactcaattttcttgtggagaaatctgcccatcaaggagagatgggatctgtcacaaaagcacttcagctcacaccaccggtaaataccttcggagatatggaaaaacgaaaacaatttggtattatgctttacaccagagcgaactatacatcccaaaccgaccctgctacaggatggcgaaaaacaatatatctcaaacgaggaggtgaaaaactcatacgagaaaatattatccagtcctttgatgatatgtactttgatggaaaagattatgtcttttcgtataccgaaaaattcggaaaagacaaaaacaatcagagaagtggaagaagttggaagctctactcaggaaaagacggcatctcccttgatcggtttcgaggaaagcgaggaaaagaatttaatgaatggagcgttgagacgattgatatagcggggatacttaatgaattatttgaagattttgacaaaaatatttctctcttggaacaaatacaacaaggcaaagatccaaagaagataaacgaacacaccgcatatgaaacattgcggtttgtaattgattcaatacagcaaatacgaaactcgggagaaaaaggtgatgaaagaaatagtgattttcttcactcacctgtgagaaatacagaaggtgagcattatgactcgagaatctatcttgatcgagaaaaagagggaatagttacagatcttcccatctcaggagatgccaatggtgcgtacaatatcgctcgaaaaggaattcttatgaaagagcacctcaagagagatctatctgaatacatatccgatgaagaatggtctgtatggctttcgggaaaaaatagatgggagaaatggatgcaagaaaatgaaaaagatttaagaaagaagaaaaaatag

SEQ ID NO: 28 - DNA, 4011 bp, Artificial Sequence
  Description of Artificial Sequence: Synthetic polynucleotide
atgaagcaggagaagaaaaccgagaagagcgtgttcagcgatttcaccaacaagtacgcgctgagcaaaaccctgcgtttcgagctgaagccggtgggtgaaaccctggagaacatgaaagacgcgtttggctacgataagaaaatgcagaccttcctgaaggaccaagagatcgaagatgcgtatcagaacctgaaaccgattctggaccgtatccacgaggaatttattacccaaagcctggagagcgaacaggcgaagcaaattccgttccacatctacgagaaaagctatcgtaagaaaagcgaaatcaccctgaagcagtttgaaaccgtggaaaagaaaattcgtgagtacttcgatgaagcgtataaacagaccgcgcaagtttggaagcaaaacgcgccgaaagataagaaaggtaagggcgtgttcaccaaggacagccacaaactgctgaccgaggtgggtgttctggaatacatccgtcagaacaccgagaagtttagcgacattctgccgaaaagcgagatcgaacaacacctgaacgttttcagcggtttctttacctattttcagggcttcagccaaaaccgtgagaactactataccaccaaggatgaaaaagcgaccgcggtggcgacccgtgtggttagcgagaacctgccgaagttttgcgacaacatcctgaccttcgagaacaagaaagaagcgtacctggcgctgtatcagagcctggcggaaaagggtaaaaccctgcaaattaaagatggtagcagcggcaagatgaaaagcctggagggcgttgacgaagcgatgtttagcatccaccacttcaacgagtgcctgagccagcgtgagattgaaaagtacaacgaagcgatcgcgaacgcgaactacctgattaacctgtataaccagctgcaagacgataagaaaaacaagctgaaactgttcaagaccctgtacaaacaaattggttgcggcgacaaggaaaccttcatcgaaaaaattacccactataccgaggaagaggcgcagaaggcgcgtaaagagaagaaagaaaaagcgatcagcctggagcaagaactgaaggagttcagcagcctgggtagcaaatacttctttggcattagcgagaacgaatttatccgtaccgttgaggatttccgtaagtatctgctggaagagaaagaagactacgcgggtgtgtattggagcaagcaggcgatcaacaacattagcggcaaatactttagcaactggcacgcgctgaaggacatcctgaaagagaagaaagttttcagcaccagcgcgagcaaggacgaaagcgtgagcatcccggagatcattgaactgaagcaactgtttgaagttctggacggtattgagaaatgggaagtgccggataacttctttaagaaaaccctgaccgaagaggttagcaaggaccaccgtgatttccagaaaaacgcgaagcgtaaagagatcattaagagcagccaaaaaccgagcgaagcgctgctgcgtatgatgtttgacgatatggtggatctgcgtgagaaattcctgagcaagaaagaggacatcctggaaaacaccaactacaccacccaggagcgtaaggacgacatcaaagaatggatggacagcggtctgcgtatcattcagattctgaagtacttcagcgtgcaagaaaagaaaatcaagggcaccccgttcgacgcgaagattaaagagggcctggataccctgctgctgagcaacgaagttgactggtttacccgttacgatcgtgtgcgtagcttcctgaccaagaaaccgcaggacgatgcgaaggagaacaagctgaaactgaactttgaaaacagcaccctggcgggtggctgggacgttaacaaagagagcgataacagctgcatcattctgaaggaagaggaaaaaaccttcctggcggtgattgcgaagagcaaaggcaaggagaaaaacaacgcgctgtttcgtaagaccgaacaaaacccgctgttcagcatcgagaacgcggaaaccatgaagaaaatggagtacaagctgctgccgggcccgaacaagatgctgccgaaatgcctgtttccgaaaagcaacccgaagaaatacggtgcgaccgaaaccgtgctggacgtttataagaaaggcagctttaagaaaaacgaggaaaacttcagcaagaaagacctgtacaccgttatcgatttctataaagaggcgctgaaacgttacgaaggttggaactgcttcgagtttcacttcaagaaaaccagcgaatacaacgacatcggcgagttttatctggatgttgaaaagaaaggctataccctggacttcgtggatattaaccgtaacgtgctgggccagtacgttgaggatggccgtgtgtacctgttcgaaatccgtaacaaagactggaacaccctgccggatggtagcaagaaaagcggcaacaccaacctgcacaccatgtactggaaggcgctgtttcaagaccgtgagaaccgtccgaaactgaacggcgaggcggaaatcttctatcgtaaggcgctgagcaaggacgaaattaagaaaaagaaagataagcacgagaaagaagttatcgagaactaccgttttagcaaggaaaaatttctgttccacgtgccgattaccctgaacttctgcctgaaggattataaaattaacgacgacatcaacgagaagctgctggagaacgaaaacgtttgcttcctgggtattgaccgtggcgaaaaacacctggcgtactatagcatcgtggacaacgagggtaacattctggaacaggataccctgaacaccatcaacggcaaggactacaacaccctgctggaggaacgtagcgaggaaatggataccgcgcgtaaaagctggcagaccatcggcaccattaaggagctgaaagatggctacatcagccaagttatccgtaagattgtggacctgagcctgcgttataacgcgtttatcgttctggaagacctgaacgtgggtttcaagcagggccgtcaaaagattgagaaaagcgtttaccagaaactggaactggcgctggcgaagaaactgaacttcctggtggagaagagcgcgcaccagggtgaaatgggcagcgttaccaaagcgctgcaactgaccccgccggtgaacacctttggtgatatggagaagcgtaaacagttcggcatcatgctgtacacccgtgcgaactataccagccaaaccgacccggcgaccggttggcgtaaaaccatctacctgaagcgtggtggcgagaaactgattcgtgaaaacatcattcagagctttgacgatatgtatttcgacggcaaggattacgtttttagctataccgaaaaattcggcaaggataaaaacaaccaacgtagcggccgtagctggaagctgtacagcggtaaagacggcattagcctggatcgttttcgtggcaagcgtggcaaagagttcaacgaatggagcgtggaaaccatcgacattgcgggtatcctgaacgagctgtttgaagacttcgataagaacattagcctgctggaacagatccagcaaggcaaagatccgaagaaaatcaacgagcacaccgcgtatgaaaccctgcgttttgttatcgacagcattcagcaaatccgtaacagcggcgagaagggcgacgaacgtaacagcgatttcctgcacagcccggttcgtaacaccgagggtgaacactacgacagccgtatttatctggatcgtgagaaggaaggcattgtgaccgacctgccgatcagcggtgatgcgaacggcgcgtacaacattgcgcgtaaaggtatcctgatgaaggagcacctgaaacgtgacctgagcgaatatatcagcgatgaggaatggagcgtgtggctgagcggcaagaaccgttgggagaaatggatgcaggagaacgaaaaagacctgcgtaagaaaaagaaatag

SEQ ID NO: 29 - DNA, 3441 bp, Artificial Sequence
  Description of Artificial Sequence: Synthetic polynucleotide
atgaaaaataacagaacaaaacacttacacccaacagggtatcaactagcaagcgagcgtatcaagcaagctccattaaacaaaaactcaaaatacatagtaacagttaagtatcctctcaaaggagatctcaagggaaaacttgagtccgagttaatagagcaatccttccgggattatgcatacgcgtatggaattcccacgctaaaggaatcaaaacctcaggtttcacttattgatttttatattgagtgtttgcgtatgggggcattttttcaaccctcatcagccaagcttcaagatttggcttcgggtgggaagcttcaagcacttataaagaaaaacattccagatcacatcctcgtgaaacttaacatgcttgagtttgtagatggtatcaccgctgactttcgcaaaatggagcaggaagagcctgcaacatttcgaaaaaaaatagctaaatggttcaaggatgatacagatccctatattgatcaggttgtggagatttatttgcagaacggccaatctcagcaaacacaatctgctgaatcggcttttttctatcgtccaaagaagaatccttccaatctaactttttatttacatccagaaattctagtggacccttcggagagtaatccccaaaaagttgtgtttgaaagcgtgagacaaatttatactgccttaaataatcagcttcagccgcctgaaaaaaagagagaagattttgatcttgaattaataggattagataaacaagcgaacgctttatcgaacttttttaacaatgtgtttaatcggttgcaaaaagatgatgtgcaatcccttatggccgagatccttgatctctccgaactttggagagggaaagagcaagagcttgaacaaagactgatccacttatctagtgttgcaaaacaggttggaaatccagcgctgggaaaaagttgggctgattacagggctatgttctctggaaggataaaatcttggtataaaaacacagtgaatcatctaaaagctagagaagaacaactacccaacctgaaagaagcagtcgaggttgtgatagcagatgtcagacaggtagttgagttaataacaaataaatcatttgatgaaagagataactcgaatcggaccgaacttctatttcattttttagaatcttgccaagcgttacttgatgcgcttgatcagaataatgaagatgtttgttttcagctgcatgctgaattgactcgtgatttcaatcttgtgcttcagcggtatgcacaagaattcctcacccttgagaattctaagaagaagaaaaaacagtttgctgaagattcagcggaagcactagagcttattcgacctaaatacgcaaaacttttctcaagattacggccccagccagcattttttggtgagcaacgggcgaaacttgtggatcgttactcggaagcagcaaagcaactatttcaactcttaactttcttacaacaactgattcttgatctctacgccctgcctcgtggtgatgcacttggagaagaaacacttttgcaaattgtggacaaggttgtgaaaagaaaaaataatgcaaatacaataaatcatcagcaactttttaaagacctgtttacccaagcaatcattcggccgtataccaaagatgaaaaagttgcttattttatcaacccaaatgcttctagattgagattgagaaaattagaaaaaagctggagattgcctgatgttgagttggtccaaatgattgaaagcaccttgcttaagtccttcaatctatcgcaagaagcgtactcacatgctgactcagaatcacttatcgatgctattgaatcctcaaaaacactcgttgcggttttattattgactcgaaaaagtacccaatattcttttgattttgaaaagattccgtccgagacgcttcgattcaagatcaaccgcctagataagaagaatagagttcaatatcttcagcgagcgacttcattcattgggacagagttgagagggtatatttctcttatttctcgatccgaagttattgatcgagcaacagtgcaactgagtaattccgataagatgtttactcctgttcgaacgaaagacaatagatggaaaatagcattgaatcacgaaaaagcagcaataggactagatcaagaggttgaaaaatttacaaagtcgggggtaaagagagaggtgcttaaacatcaaaccttagatatcaagacctcaagataccaacttcagtttctagaatggttgcacaaaactccaaaaaagaaacagcatctcaatatcgcattgaatgaaccctcacttattgctgagaaaaaatatcgaatcaattggactgtgcaaaatcaaattttagtcccagaatatgttttgcttgaatctggggtatttctttcaataccttttacgattagtccagcgaaagataataataaaagcttctctcgttatttgggactagacttaggggaatttggtgttgcttgggcagttcttgggattaaagataacaggccgtatttagtgcagacgggcatgcttcaagatcctcaattacgagcaattgctaatgaagtagctgtcatgaaggcgagacaagtaaccggaacttttggcgttccaagctctcgccttcaaagacttcgggaaagcgcagtgcattcgttagtgaatcaaattcattctttggtgttgcggtatggagcaaaaatggtgtttgaacgacaggttgatgcctttcaaacaggttcaaatcgagtgaaaaaaatatatgcttcattgaagcaggggaatatatttgggcgcaaagagatagataaatcaaactataaaagatattggagttatcgagacggtcattttatgggcagcgaagtaagttcctggggcacaagttatttttgtccacattgtagagagtttcttcatgatcttccaaaagagaaggatgcgtatgagctagtgaaagattccccagaagaattgactaggcttcgagtatattcggtgaaacaaacaggagaaaaatattatggatatgttgaaggaaatagcagtccaaaagaacaagttcttgcatttgctcgcccaccatatcaaagtgacgcgttacttttgttatcaaaacagggtaaaaatctcaacttatcacaaagtttgaaaaccgaacgcggtggtcaagcggtctttgtatgccccaaattttcatgtttgaggacttatgatgctgataagcaagcagcggtaaatattgcgatgcgcaaatgggctgaagacgtatttattgctactaaaggtaagcctccaaagcaaagggatgagaattattttagaatgaggaaagattttgaaagaaaattatataaagatttgaatgaatacccaaccgttaaaatgggtgagtag

SEQ ID NO: 30 - DNA, 3441 bp, Artificial Sequence
  Description of Artificial Sequence: Synthetic polynucleotide
atgaaaaacaaccgtaccaagcacctgcacccgaccggttaccagctggcgagcgagcgtattaaacaagcgccgctgaacaagaacagcaaatacatcgtgaccgttaagtatccgctgaaaggtgatctgaagggcaaactggagagcgaactgattgaacagagcttccgtgactacgcgtatgcgtacggtatcccgaccctgaaggagagcaaaccgcaagtgagcctgatcgacttttacattgaatgcctgcgtatgggcgcgttctttcagccgagcagcgcgaaactgcaagatctggcgagcggtggcaagctgcaggcgctgatcaagaaaaacattccggaccacatcctggtgaaactgaacatgctggagttcgttgacggtattaccgcggattttcgtaagatggaacaagaggaaccggcgaccttccgtaagaaaatcgcgaagtggtttaaagacgataccgacccgtacattgatcaggtggttgagatctatctgcagaacggccaaagccagcaaacccaaagcgcggaaagcgcgttcttttaccgtccgaagaaaaacccgagcaacctgaccttctatctgcacccggaaattctggtggacccgagcgagagcaacccgcaaaaagtggtttttgagagcgttcgtcagatctacaccgcgctgaacaaccagctgcaaccgccggaaaagaaacgtgaggacttcgatctggaactgatcggtctggataaacaggcgaacgcgctgagcaacttctttaacaacgtgtttaaccgtctgcagaaggacgatgttcaaagcctgatggcggaaattctggacctgagcgagctgtggcgtggcaaggagcaggaactggagcaacgtctgatccacctgagcagcgtggcgaaacaggttggtaacccggcgctgggcaagagctgggcggattaccgtgcgatgttcagcggccgtatcaagagctggtataaaaacaccgtgaaccacctgaaagcgcgtgaggaacagctgccgaacctgaaggaagcggttgaggtggttattgcggacgttcgtcaagtggttgagctgatcaccaacaagagcttcgacgaacgtgataacagcaaccgtaccgaactgctgttccactttctggagagctgccaggcgctgctggacgcgctggatcaaaacaacgaggatgtgtgcttccagctgcacgcggaactgacccgtgactttaacctggttctgcagcgttacgcgcaagagttcctgaccctggagaacagcaagaaaaagaaaaagcagtttgcggaggatagcgcggaagcgctggagctgatccgtccgaagtatgcgaaactgttcagccgtctgcgtccgcagccggcgttctttggcgagcaacgtgcgaaactggttgaccgttacagcgaagcggcgaagcagctgttccaactgctgacctttctgcagcaactgatcctggacctgtatgcgctgccgcgtggtgatgcgctgggtgaagaaaccctgctgcagattgtggataaagtggttaagcgtaaaaacaacgcgaacaccatcaaccaccagcaactgttcaaggacctgttcacccaagcgatcattcgtccgtacaccaaggacgagaaagttgcgtatttcattaacccgaacgcgagccgtctgcgtctgcgtaagctggagaagagctggcgtctgccggacgtggaactggttcagatgatcgagagcaccctgctgaagagctttaacctgagccaagaggcgtacagccacgcggacagcgaaagcctgatcgatgcgattgagagcagcaaaaccctggtggcggttctgctgctgacccgtaagagcacccagtatagcttcgattttgaaaaaattccgagcgaaaccctgcgtttcaagatcaaccgtctggacaaaaagaaccgtgtgcagtacctgcaacgtgcgaccagctttattggcaccgagctgcgtggctatatcagcctgattagccgtagcgaagtgatcgaccgtgcgaccgttcagctgagcaacagcgataaaatgttcaccccggtgcgtaccaaagacaaccgttggaagattgcgctgaaccacgaaaaggcggcgatcggtctggatcaggaagttgagaagttcaccaaaagcggcgtgaaacgtgaggttctgaagcaccaaaccctggacatcaaaaccagccgttaccagctgcaatttctggaatggctgcacaagaccccgaaaaagaaacagcacctgaacattgcgctgaacgaaccgagcctgattgcggagaagaaataccgtatcaactggaccgtgcagaaccaaatcctggtgccggaatatgttctgctggagagcggtgttttcctgagcattccgtttaccatcagcccggcgaaggataacaacaagagcttcagccgttacctgggcctggacctgggcgagtttggcgtggcgtgggcggttctgggtattaaagataaccgtccgtatctggttcagaccggcatgctgcaggacccgcaactgcgtgcgatcgcgaacgaagtggcggttatgaaggcgcgtcaagttaccggcacctttggcgttccgagcagccgtctgcagcgtctgcgtgagagcgcggtgcacagcctggttaaccaaattcacagcctggtgctgcgttacggtgcgaaaatggtgttcgaacgtcaggttgacgcgtttcaaaccggcagcaaccgtgttaagaaaatctatgcgagcctgaagcagggtaacattttcggccgtaaggagatcgataaaagcaactataagcgttactggagctatcgtgacggtcactttatgggcagcgaggtgagcagctggggcaccagctacttctgcccgcactgccgtgaatttctgcacgacctgccgaaggaaaaagatgcgtatgagctggttaaagatagcccggaggaactgacccgtctgcgtgtgtacagcgttaaacagaccggtgaaaagtactatggttatgtggagggcaacagcagcccgaaggaacaagttctggcgtttgcgcgtccgccgtaccagagcgatgcgctgctgctgctgagcaagcaaggcaaaaacctgaacctgagccagagcctgaaaaccgagcgtggtggccaggcggtgttcgtttgcccgaagtttagctgcctgcgtacctacgacgcggataaacaagcggcggtgaacattgcgatgcgtaagtgggcggaagatgttttcatcgcgaccaagggtaaaccgccgaaacagcgtgacgagaactatttccgtatgcgtaaggactttgagcgtaagctgtacaaagatctgaacgagtatccgaccgttaagatgggcgaatag

SEQ ID NO: 31 - DNA, 3507 bp, Artificial Sequence
  Description of Artificial Sequence: Synthetic polynucleotide
atggcgcgtaaggacaaataccgcgggctgaccggctaccgtctgcaccagaagcggctggagcgctcgggtaagcagggtattcgcaccattaagtatccgctcgttggcgcgacggaggagcaccatgagcaattcgtgagtgacgtcatccacgactacaacgcgcaggtcggcgcgctgaacctgcccgagtggctggcgcagtatcgcggcgagcagacgttctacagtctcttcgatctgtggctggacttgctgcgcgccggattcgtgtgcgcgcccagcagcgcgcgccttatggagcgcgtctgctggttagcggatctgccgtcgccgcgcgcccagctgcgcgatcagatgcaagaggtcaaccccgatttctataccgcactctctgagaacggattccaccacttcgtggacacggtggtactcggcaaggagatgcgctcgagcaaaagcgagcgctcgttcgtgcgcgatctgaccacgtgtgctaccgatgcagcacaggaatacgcggagcgcgaagcgcgtacgatctaccacgccctctacggcagcgaccgcacggaacaggagcgctactggcgcgagcactatggtgttgataaaacactctttcagccgacgacccgccgcaactttgccgcatacccggtgccggctctccagctatcaccggatgcagcgcccggcgcactgctacagcggtaccgatcgctggtgcagacgcagctgagtgcacagcaggcagagcgtgttgccacgcaggagacgcagctcttggaggacatgctcggtatcgataacaacgccaacgcgctctcgaacgtattcaacgagtttctccgcgaggtgcgtaccgagacaggccgtgctgcgatcgctgacgatatgcagcagttcagtcgcgcgtgggacggacgacgctcggagttggaagagcgcctgcgctggctcggcgagcgtgcggcgcagctgccggcgcagccgcggctggcgaatagctgggcggactaccgcaccagcgtggccggcaaactccagagctgggtgtcgaacgtggcacggcaagagcacgtcatccgtccgcgactggagcagcaacgcagtgagctcgacgacctggccgagcggctacgcgcgctcagcgatgaggagaccgggctgccggctaccgttgagcaggcacaggcagcgctcgacgccgcgctggcggcagagcaatcggatgagtcgacgctgatggtctaccgcgatgcgctcgctgacgtgcgtgcggcactcaatgaaggtcagcatacgctgcaaatgcacgagcacggcatcgaacacgtggacactgacagcagctgggcatcggacacgtggccgacgctccaccagccggtaccgcaggtgccccagttcccgggcgtgacgaaggcgtacgcgtacacgaagtacgtgcacgcgctcgagctgttgcgcagcggtgctgccgtacttgagcgggccgccgccgatgccagtgagcgggaggccgttcagctctcgcgcgaggagatgctgcgccgcctgacgaacgtggcgcagcagtacgcacgctgcaacagccagcggttccgtgacctgatcggtggcgtattccaacggcacgaggtgctactcaacgatgttgttgaacggggagcggtgtactaccagtcgccgcgcgcccgcaacaagaagccgctggttgaactgagtcacaccgacgagcagttgcacgcggtgatcaccgatctcgtctggaagtgtgcgccgtactgggaacgcatgtgggggcagatcgaggaggtcgtcgatgcgattgactttgagcgcgtccggctcggcatgctctgtgcgctgtatccggacaccactgccgatattagtgatgtgtcagagacgctgttcacccgagctggcgggtaccagcgcgcctacggcactgagttgaccggcaccacgctctcgaattgtatacagcgggtcattctagcggagatgaaaggcgcggcgcagcggatgagccgtgagtggtttgtggtgcgctacacggtgcagatcgtcaaagcggacgagctgtatccgctgatctatcaacccggctctacgggcggccgcggcacatggcacatcaccgatcgacagaacgtgcgtcgaagtgcagcagacacgccgccggtgtaccggaaagtcgggaagaacctcccgcacgacaccgcgcttgccggtttcgacggcgcagaagtaactgatacgcagcgtctcctctcgattcgcagctcgcgctatcagctacagttcttgcaagaccagcttcacgccggcagtgaacacatgcggcgacgtttcagctggagcatcgccgagtactcattcatttgtgaggatacgtatacggccgcgtgggatacagagcgcggcaccgtttcgctcgagcggcagccgagcgctcgtcgtctgttcgtttccattccgttccagctgcggcggctagaagccgctgatggtcgatcgtcctatcagccaaagagcggcttgccgtacagctacctgttggggctcgacgtgggtgagtacggtatcgcgtactgcctgctagagccggagaccggcgagtggcggacgagcggtttctttgcagacgatgcgatacgcaagatccgccagtacgtttccaggcagaaagaggcacaggtacgcagcactttcagtgcgccgtcgtcagaacttgcacgtatccgcgagaacgcgatcaccgcgctacgcaatcgcgtgcacgatctgaccgtacgctacgatgcgcggccggtgtacgaattcaatatctctaactttgagagtggttctaatcgcgttgccaagatctatcggtccgtcaaaaccgctgatgtgcacgctgacaacgatgcggatcaagcggagcgcgacctcgtgtggggtagtgccagcaagctgaccggcagcgagatcggggcgtacggtaccagttacgtatgcagcaagtgtcacgcctcgccgtatacggctattcaaccaatgcagcaatccgcatacgagtgggagtgggttggtcagcagcagcggatcgtgcgcatttacacacctgaaaacggtgctgcgcttgggcacatcgatattagacagtacaagccaagtgatacgttgccgtcggtggatgcactccgctttttgaaggcgtacgcgcggccgccgctcgaggcgctcgtacagcgttcgggctttacggatcaggacacgatagaccggctccacgcgtacgtacaagagcgtggtgacagtgcggtgtacacctgcccgttctgtgagcacacagcagattgcgatgtgcaggcagcgctcatcgttgctgtgaagtatgcgatcaagcagcacggatcgccgagtggcgagaagggtgaagtgacgctggaagacgttagcgcatacctccgtggtcacgaggtgcagcccgtctcattcgcataatag

SEQ ID NO: 32 - DNA, 3504 bp, Artificial Sequence
  Description of Artificial Sequence: Synthetic polynucleotide
atggcgcgtaaggacaaataccgtggtctgaccggctatcgtctgcaccaaaagcgtctggaacgtagcggtaaacagggcatccgtaccattaagtacccgctggttggtgcgaccgaggaacaccacgagcaattcgtgagcgatgttatccacgactataacgcgcaagtgggtgcgctgaacctgccggaatggctggcgcaataccgtggcgagcagaccttctatagcctgtttgatctgtggctggacctgctgcgtgcgggttttgtttgcgcgccgagcagcgcgcgtctgatggaacgtgtttgctggctggcggatctgccgagcccgcgtgcgcagctgcgtgatcaaatgcaggaagttaacccggacttctacaccgcgctgagcgagaacggtttccaccactttgtggacaccgtggttctgggcaaggaaatgcgtagcagcaaaagcgagcgtagctttgttcgtgatctgaccacctgcgcgaccgatgcggcgcaggaatatgcggagcgtgaagcgcgtaccatctaccacgcgctgtatggtagcgatcgtaccgagcaagaacgttactggcgtgagcactatggcgttgacaaaaccctgttccagccgaccacccgtcgtaacttcgcggcgtacccggtgccggcgctgcaactgagcccggatgcggcgccgggtgcgctgctgcagcgttatcgtagcctggtgcaaacccaactgagcgcgcagcaagcggagcgtgttgcgacccaagaaacccagctgctggaggatatgctgggtatcgacaacaacgcgaacgcgctgagcaacgtgttcaacgagtttctgcgtgaagttcgtaccgagaccggtcgtgcggcgattgcggacgatatgcagcaattcagccgtgcgtgggatggtcgtcgtagcgaactggaggaacgtctgcgttggctgggcgaacgtgcggcgcaactgccggcgcagccgcgtctggcgaacagctgggcggactaccgtaccagcgttgcgggcaagctgcaaagctgggttagcaatgttgcgcgtcaggaacacgtgatccgtccgcgtctggaacagcaacgtagcgagctggacgatctggcggaacgtctgcgtgcgctgagcgatgaggaaaccggtctgccggcgaccgttgagcaagcgcaagcggcgctggatgcggcgctggcggcggaacagagcgacgagagcaccctgatggtgtatcgtgatgcgctggcggatgttcgtgcggcgctgaacgagggtcaacacaccctgcagatgcacgaacacggcattgagcacgtggacaccgatagcagctgggcgagcgatacctggccgaccctgcaccaaccggtgccgcaagttccgcagtttccgggtgtgaccaaggcgtacgcgtataccaaatacgttcacgcgctggaactgctgcgtagcggtgcggcggtgctggagcgtgctgcggcggacgcgagcgagcgtgaagcggttcagctgagccgtgaggaaatgctgcgtcgtctgaccaacgtggcgcagcaatatgcgcgttgcaacagccaacgtttccgtgatctgatcggtggcgtgtttcagcgtcacgaagttctgctgaacgacgtggttgagcgtggtgcggtttactatcaaagcccgcgtgcgcgtaacaagaaaccgctggttgagctgagccacaccgatgagcagctgcacgcggtgatcaccgacctggtttggaaatgcgcgccgtactgggaacgtatgtggggtcaaatcgaggaagtggttgatgcgattgacttcgagcgtgttcgtctgggcatgctgtgcgcgctgtatccggataccaccgcggatattagcgacgtgagcgaaaccctgtttacccgtgcgggtggctaccagcgtgcgtatggtaccgagctgaccggcaccaccctgagcaactgcatccaacgtgttattctggcggaaatgaagggcgcggcgcagcgtatgagccgtgagtggttcgtggttcgttacaccgtgcaaatcgttaaggcggacgagctgtacccgctgatttatcaaccgggtagcaccggtggccgtggtacctggcacatcaccgatcgtcaaaacgttcgtcgtagcgcggcggacaccccgccggtgtaccgtaaggttggtaaaaacctgccgcacgataccgcgctggcgggttttgatggtgcggaagtgaccgacacccagcgtctgctgagcattcgtagcagccgttatcaactgcagtttctgcaagatcaactgcatgcgggtagcgagcacatgcgtcgtcgtttcagctggagcatcgcggaatacagctttatttgcgaggatacctataccgcggcgtgggacaccgaacgtggtaccgttagcctggagcgtcaaccgagcgcgcgtcgtctgttcgttagcatcccgtttcaactgcgtcgtctggaagcggcggatggccgtagcagctaccagccgaagagcggtctgccgtacagctatctgctgggcctggacgtgggtgaatacggcattgcgtattgcctgctggagccggaaaccggcgagtggcgtaccagcggcttctttgcggacgatgcgatccgtaaaattcgtcagtacgtgagccgtcaaaaagaggcgcaggttcgtagcacctttagcgcgccgagcagcgaactggcgcgtatccgtgagaacgcgattaccgcgctgcgtaaccgtgtgcacgatctgaccgttcgttacgacgcgcgtccggtttatgaattcaacatcagcaactttgagagcggtagcaaccgtgtggcgaagatttaccgtagcgtgaaaaccgcggatgttcacgcggacaacgatgcggaccaggcggaacgtgacctggtttggggtagcgcgagcaaactgaccggcagcgagatcggtgcgtacggcaccagctatgtgtgcagcaagtgccacgcgagcccgtacaccgcgattcaaccgatgcagcaaagcgcgtatgagtgggaatgggtgggtcagcaacagcgtatcgttcgtatttataccccggaaaacggtgcggcgctgggtcacatcgatattcgtcagtataaaccgagcgataccctgccgagcgttgacgcgctgcgtttcctgaaagcgtacgcgcgtccgccgctggaggcgctggtgcaacgtagcggttttaccgatcaggacaccatcgatcgtctgcacgcgtacgtgcaggaacgtggcgacagcgcggtttatacctgcccgttctgcgagcacaccgcggattgcgatgtgcaagcggcgctgattgtggcggttaagtacgcgattaaacagcacggtagcccgagcggcgagaaaggcgaagtgaccctggaagacgttagcgcgtatctgcgtggccacgaggtgcagccggttagctttgcgtag

SEQ ID NO: 33 - DNA, 3738 bp, Artificial Sequence
  Description of Artificial Sequence: Synthetic polynucleotide
atgaggagacaattagaagattttgccaatctttatgaaatttccaaaaccttgcgttttgaattgaggcctattggaaaaacgcgtaaaatgcttgaggaaaataaagtatttgaaaaagatgaggcagtagctcaaaattaccaagaagcaaaaaaatggctggataaattgcatagagattttattagccgctctcttgaggatttaaaaataaattccgaacttctggaagaacacaaacaggcttattttgactacaaaaaagaaaaaaattcttccaacagaaataattttgaagaaaaatccaaaaagctgagaaaagaaattttattgaatttttgccaaaaaggagaagaattgagagataattacttgagagaaataaaagatgaaaaaatcaaaaagagagttcgaaagctgagaaacttggatattctttttaaagtggaagtttttgattttttaaaacaaagatacccggaagctgttgttgacgagaaaagtattttcgatgcctttaatagatttagtacttattttacaggtttccacgagacaagaaaaaatttctataaagacgacggtactgccaccgctattcctaccagaattgtaaatgaaaacctacccaagtttcttgataatttggaagtttacaatagatattacaaagaaggcattggagatttgtttacaggagaagaaaaaaatattttcaacttggaattttttaatgattgtttttctcaaagagagattgattcttacaacagaattatttccgaaataaatttaaaaattaaccaaaaacgccaaacagcggaaaataagaaaaattttccctttcttaaaacgcttttcaagcaaattttgggagaagaagagaaacaggaaaccgagtctcttgattatatagagataacccgggatgaagacgtgtttccggctttgaagagctttgtagaagaaaacgagaggcaaactcctagggccaataagcttttcaacaggttaattcaagatcaaaaagagcaaaaaggcggttttgatatttccaatgtttttgtagctggtagatttattaatcagatttccaataaatactttgcagactggaacaccattagaagtatttttattgaaaagggaaaaaagaaattaccggagtttgtttctctgcaagagctcaaagaaaaactccaaagcatagagatagaaaaaagcgaattatttagagagaagtataaagatatatataaaaaccgaggggataattttattatctttcttgagatatggcaaaaagaatttgaagagagcctaaaaagatacagagaaagcttggaagaaaccaagcaaatgcttgagcagcaagaaggctatcaaagcaaggaaagttccgaacagaaaaactcaattcgccgttattgtgaaaatgcgctctctatttatcaaatgataaagtatttttccctggaaaaaggcaaggaaagggtttggaatccggacaaactggaagaagaccccggattttacgagcttttcaaggactattaccaagatgctcatacttggcaatactataacgaatttcgaaactatttaaccaaaaagccttatagtcaagataaggttaaattgaattttggaagcggaaccttattgcaagggtggccagatagtccggaaggcaatacccagtataaaggttttatttttaaaaaaaataaaaaatattttttaggcataacaaattatcctaagatgtttaatgaaaagcgtcaccctgaagcttatgataatgatattgatccttattataagatgatttacaaacaattagacagcaaaaccatattcggttctttgtatttaggaaaatttggaaataagtacaaagaagataaaaaaagaatggttgactttaagctacaaaacaggataagagctatattaaaagagaaggtcgagtttttccctcgattgcaaaccattatagataaaattgaaaatcataaatattcgaatacaaaggatattgctgtggatatttctaagataaagttatacaacattttttttatagaaacaaactctttgtatgttgaacaaggtaagtatgagatagacaataatacaaaaaatttgtatctctttgaaatttacaacaaagattttgcaaagaaggcagaaggaaaaaagaatctgcacacctattactgggaggagattttttcccaaagaaatcaagataatccgatcatcaaattaaacggccaagccgaggtatttttcagaagagcctctttggatccggaagttgacgaagaaagaaaagcgcctcgggaagttgtaaataaagaaagatacactgaagacaaaatgttttttcattgtcccttgacgcttaattttgccaaaggtcgagcggatgggtttagtataaaggcgagggagtatttgctcgaaaatccggaggtgaacattatcggcatcgatcggggggaaaagcatttagcctattattccgtagcggaccaagaagggaatattttggaaatagattcccttaataaaatcaatgaagttgactatcataaaaagcttgataagttggaaaaagcaagggatgaggctcgcaaaacttggcaggatatagccaagatcaaagaaatgaaacaaggatatatttcccaggttgtaaagaaaatttgcgacttaatgataaaacacaatgctatagtggtttttgaagatctcaacctcggctttaagtgcggaagatttgccatagagaagcaggtttatcaaaacttggagctggctttggccaaaaaattgaattatttggttttcaaagagagggaagcggaggagcttggcagtttcaggcatgcgtttcaattaactcctcaaatatctaatttcaaagatattaaaaaacaatgcggttttatgttttatattcctgccagatacacctccgctatttgccctaactgcggtttccgcaaaaatatttccactcccgttgacaaaaaagctaaaaacaaagaatatcttgaaaagtttcaaatttcttacgagcaagatagatttaaatttgcttacaagaaaagagatgtccttgagagagggaggggaaaccccggtcaaaatagccggcgcctttttgaggaaaaagcttcaaaagatgattttattttctactccgatgtttccagattacagtttcaaagaaataaagacaatcggggaggcgaaacaaagtggcgcgagccgaacgaagagctgaagagaattttcaaagaaaacgggattgacatcaataaagacattaacaagcaaatcaaagaaggagattttgaaaatgacgctttctacaagagaattattcacaccattcgtttaatattgcaattgagaaacgccataacaaaaaaagacgagcaaggaaatgaaattgaagaagaaagccgggattttattcagtgcccctcttgtcattttcattcagaaaacaatcttttggccttaagcgagaaatacaaaggggatgaaccgtttcaattcaacggcgatgccaatggagcatataacatagctcgcaagggaagtcttattttaagcaagatttcaaattttaacaaaacagagggtgatttaagcaaaatggataaccaagatttgaccattacccaagaagaatgggataaatttgcgcaaaataaatag

SEQ ID NO: 34 - DNA, 3738 bp, Artificial Sequence
  Description of Artificial Sequence: Synthetic polynucleotide
atgcgccgtcaactggaggactttgcgaacctgtatgagattagcaagaccctgcgctttgaactgcgtccgattggtaaaacccgtaagatgctggaggaaaacaaagtgtttgagaaggacgaagcggttgcgcagaactaccaagaggcgaagaaatggctggataaactgcaccgtgacttcattagccgtagcctggaggatctgaagatcaacagcgaactgctggaggaacacaaacaagcgtactttgactataagaaagaaaagaacagcagcaaccgtaacaacttcgaggaaaagagcaagaaactgcgtaaagagatcctgctgaacttttgccagaaaggcgaggaactgcgtgataactacctgcgtgagatcaaagacgaaaagattaagaaacgtgttcgtaagctgcgtaacctggatattctgttcaaggttgaggtgttcgactttctgaaacagcgttatccggaggcggtggttgatgagaagagcatcttcgatgcgttcaaccgtttcagcacctactttaccggcttccacgaaacccgtaaaaacttttataaggatgatggcaccgcgaccgcgatcccgacccgtattgtgaacgagaacctgccgaagttcctggataacctggaagtgtacaaccgttactataaagaaggtattggcgacctgtttaccggcgaggaaaagaacatcttcaacctggagttctttaacgattgctttagccagcgtgaaattgacagctataaccgtatcattagcgagatcaacctgaaaattaaccagaagcgtcaaaccgctgagaataagaaaaacttcccgtttctgaaaaccctgttcaagcagatcctgggtgaggaagagaagcaagaaaccgaaagcctggattacatcgagattacccgtgacgaagatgtgtttccggcgctgaagagcttcgttgaagagaacgaacgtcagaccccgcgtgcgaacaagctgtttaaccgtctgattcaggatcaaaaagagcaaaagggtggcttcgacatcagcaacgtgtttgttgcgggtcgtttcatcaaccagattagcaacaaatactttgcggactggaacaccatccgtagcatcttcattgagaagggcaagaaaaagctgccggaatttgtgagcctgcaggagctgaaagaaaagctgcaaagcatcgagattgagaagagcgagctgttccgtgaaaagtacaaggatatttacaagaaccgtggcgacaactttatcatcttcctggaaatctggcaaaaggagttcgaagagagcctgaaacgttaccgtgaaagcctggaagaaaccaaacagatgctggagcagcaagaaggttaccagagcaaggagagcagcgaacagaagaacagcatccgtcgttattgcgagaacgcgctgagcatctaccaaatgattaagtatttcagcctggagaaaggcaaggaacgtgtttggaacccggataaactggaagaggacccgggcttttacgaactgttcaaggattactatcaggacgcgcacacctggcaatactataacgagtttcgtaactacctgaccaaaaagccgtatagccaggataaagtgaagctgaactttggtagcggcaccctgctgcagggttggccggacagcccggagggtaacacccaatacaaaggcttcatcttcaaaaagaacaagaagtactttctgggcatcaccaactatccgaaaatgttcaacgagaagcgtcacccggaagcgtacgacaacgatattgacccgtactacaagatgatctacaagcagctggatagcaaaaccatctttggtagcctgtacctgggtaaattcggcaacaaatataaagaggacaaaaagcgtatggtggacttcaagctgcaaaaccgtatccgtgcgattctgaaagagaaggttgagttcttcccgcgtctgcagaccatcattgacaaaattgaaaaccacaagtacagcaacaccaaagacatcgcggtggacatcagcaagatcaagctgtacaacatcttctttatcgaaaccaacagcctgtacgttgagcagggtaagtacgaaatcgataacaacaccaagaacctgtacctgtttgaaatctataacaaagacttcgcgaaaaaggcggagggcaaaaagaacctgcacacctactattgggaagaaatcttcagccagcgtaaccaagacaacccgatcattaaactgaacggtcaggcggaagtgttctttcgtcgtgcgagcctggacccggaagtggacgaagagcgtaaggcgccgcgtgaggtggttaacaaggagcgttacaccgaagataaaatgttctttcactgcccgctgaccctgaactttgcgaagggtcgtgcggacggcttcagcattaaagcgcgtgaatatctgctggagaacccggaagtgaacatcattggtatcgaccgtggcgagaaacacctggcgtactatagcgttgcggatcaagagggcaacatcctggaaattgacagcctgaacaagatcaacgaggttgattaccacaaaaagctggacaaactggagaaggcgcgtgatgaagcgcgtaaaacctggcaggacatcgcgaagatcaaggaaatgaagcagggttacatcagccaagtggtgaagaaaatctgcgatctgatgattaaacacaacgcgatcgtggttttcgaggacctgaacctgggttttaagtgcggccgtttcgcgatcgagaaacaggtgtaccaaaacctggaactggcgctggcgaaaaagctgaactatctggtttttaaagagcgtgaagcggaagagctgggcagctttcgtcatgcgttccagctgaccccgcaaattagcaacttcaaggacatcaagaagcagtgcggtttcatgttttacattccggcgcgttataccagcgcgatctgcccgaactgcggctttcgtaagaacattagcaccccggtggacaaaaaggcgaaaaacaaggagtacctggaaaaattccagatcagctatgaacaagatcgtttcaagtttgcgtacaaaaagcgtgacgttctggagcgtggtcgtggcaacccgggtcagaacagccgtcgtctgtttgaagagaaagcgagcaaggacgatttcatcttctacagcgatgtgagccgtctgcagttccaacgtaacaaggacaaccgtggtggtgaaaccaaatggcgtgaaccgaacgaagagctgaaacgtatcttcaaggagaacggtatcgatattaacaaggacatcaacaagcagatcaaagagggtgattttgaaaacgatgcgttctacaagcgtatcattcacaccatccgtctgattctgcagctgcgtaacgcgatcaccaaaaaggatgagcaaggcaacgaaattgaagaggaaagccgtgactttatccaatgcccgagctgccacttccacagcgaaaacaacctgctggcgctgagcgagaaatacaagggtgatgaaccgttccagtttaacggtgacgcgaacggcgcgtataacatcgcgcgtaaaggtagcctgatcctgagcaagattagcaacttcaacaaaaccgagggcgacctgagcaagatggataatcaagacctgaccatcacccaagaagagtgggacaagttcgcgcagaataaatag

SEQ ID NO: 35 - DNA, 3447 bp, Artificial Sequence
  Description of Artificial Sequence: Synthetic polynucleotide
atgacagaaaatatatccactgaaaaacaaactgcatataaaatacagaactcaagtgacaagcacttctttgcatcctttctaaatcttgcagtgaataatgtagaaaatgcttttgatgaatttgcaaaacgattaggagtttcaaattctaataaaaaaggcgagagatataaacctgatgaaagcattaaacagtttttcaaacctgagttatcattaactgattgggaaaaacgtgtggatatgcttgaacaatattttccgcttgtaagttaccttaagggaaatgtaacagataataatgaaaaggatagcaaatctaaaatacttaaatgtgatttttcatcacatgatgaaatgaagaaagcatttgctaattatctcacatatttagtaaaagctttagatgatttgagaaattattatacccatttttatcatgatcccataaaatttaaacctgaagataaaaagttttatgagttcctggatgagctttttgtagaggtaataaaagatgtaagaaaaaagaagaagaaatctgataaaactaaagaagcccttaaagatgaacttgaaattgagtttgaggagcgcatgaaagacaaaagtgctgctctcgaaaaaatggataaagatgcaggtaaaaaggtcaaaaatagaagcgaagatgagctgagaaatgctgtaatgaatgatgctttcaagcatctgattgcaaaggataaggatgaatattctctaatagaaaggtatcaggcatttcctgagaatctggatgctcctatttcagaaaagtctctcatgtttttgtgctcatgctttttatccagacgggatatggagctgtttaaagctcgaattacaggttttaaaggcaaaatggttgaaggagaagatagtttaaaatacatggccacacattgggtatataattacctgaattttaaagggcttaaacgaaaaatcaacacccgttttgagaaagaaaacctcctgtttcaaattgttgatgaactgagcaaagtaccggactgcctttatcgggttattaaggataaaaacgaattcttactcgatataaacaagttttataaacaaacaaaaggcgaggctgaaagtccggaaaacgaagaggtggttaatccaataataagaaaacggtttgaggataaattcaactactttgccttacgctaccttgatgaatttgccggttttgaaaacctgaaatttcagatatacgccggaaactacctccatcacaagcaagaaaagacaagtgcccaaacgcaacttaaaacagatagaaaaatcaaggaaaaaattaatgtttttgggaaattatctgatgtcaacaaagcaaaggcaaacttttttgcaaacaaaaccgaggatagcgacatggatgagggcttggaagaatatccaaatccctcatacaacattaatggagggagcattttgatacacttaaatttgaacaaatatagatatgggcaagaattccatgaattgaaacagttgcgtattgaaaaggaaaaacgtggggagaataaaacagataaaatttcaattattaaagatttgtttgaagataatactgaaatcaaagaagaagattgggtcttccctgttgccttattgtctctaaatgaactgcccgctttgttgtatgaaatgctcgtaaataagaaaagttcgaaggatattgaacaaatcattgcagacaggattgtttcgcattacaagaaaataaaagattttgaaggtactgcagatgagttaaaagacaaaaatctgcctgttaatttacgtaaagcttttggtgctgatgataaaaatactgataaactggaaaatgccattaccaaggacatagaagcaggagaagataagcttcagctgatcaaagagaatacaagagaaatgcgcagtaataaccgcaaatatgtattttatttaaaagagaaaggggaagaagcaacatggctggcaaaggacattaagcgatttatgcctgaaaatgcaaaaaatcaatggaagtcgtataatcacaatgaattgcaaaaggggctggcttattatgaacttgaaagacaaaatgttttggctctgcttgaatcaaaatgggatatggattcctgtcacccacactggggtgaagacctgaaagaactttttattacgcacagccgttttgatgatttttataaagcttatatgctttgtcgtcaaggatttttggagcaatttaaaaccctggttattaggaataaatcggacaaaaagcttctgaataaagttcttaaagatgtttttattccttataaaaaacgattttttgtaatcaatagccttgaaaatgaaaagaaggcattgttaagtcatcccattgtgttgccaagaggcttgtttgataataaaccaactttcattaaaggggtttcgcttgaaaatgatccgtcacgctttgcaaactggtttgcatatttacgacaggaagccaaaaacgatcatcaggtattctatgattttgaaagagactatgttaaagctttttccgagctgaaagataaaagtaagtacaacaataataagcacttcaatttcaaggtagattcagaaataagaatgtgtttgcaaaatgatcttgtcttaaagttgattgtgaaaaagctttttaaaggtatttttgatgttgatgaaaatataaagttaaatgatttctatcttgaaaagacagaagttgcaaaacagagagagcaagctcttgatcagaataagcgattaaaaggagatgatggagatgtgatatataaggaagaccacttgtttcgtaaaacatttgctaaagattttctaaacggcaaattgcatttcgacaaatttaaattgaaagattttggtaaagctctggtatttgcagcagatgaaaaagtaaaaactttagtttcttattcggaaaacgcctggacacaggaagagttacagaaggaattacatacaaataccgactcttatgagcgcatacggcaagatgagttttttaaaaaaattcatgagcttgaagaatctatttggcaaaagcataaacatgaaagagaaaagttacaagacaaaagtggtaatgaaaatttcaataattatgtaaaagttggagtgctggaaaagctgaacgattcatttaaggatgaatttgaaaacttatataaagataaaaaaaataaaagaattcaaaaactcaggcaatgtaaccatgtcgttcaaaaagcatactgccttgtgcagcttagaaataagttttcacacaatcagttgcctccaaaacaactgtttgattttatgactgaaaccctggctgaaaaagacaagcaaacatacagccgttattttatggatgttactgataaaatggtgcaggaatttaagccactggtttag

SEQ ID NO: 36 - DNA, 3447 bp, Artificial Sequence
  Description of Artificial Sequence: Synthetic polynucleotide
atgaccgagaacatcagcaccgaaaaacagaccgcgtacaagattcaaaacagcagcgacaagcacttctttgcgagcttcctgaacctggcggttaacaacgtggagaacgcgttcgatgaatttgcgaagcgtctgggtgttagcaacagcaacaagaaaggcgagcgttacaaaccggacgaaagcattaaacagttctttaagccggagctgagcctgaccgattgggaaaagcgtgtggacatgctggagcaatacttcccgctggttagctatctgaagggtaacgtgaccgataacaacgaaaaggacagcaaaagcaagatcctgaaatgcgattttagcagccacgacgagatgaagaaagcgttcgcgaactacctgacctatctggttaaagcgctggacgatctgcgtaactactatacccacttttaccacgatccgattaaattcaagccggaggacaagaaattctatgaatttctggatgagctgtttgtggaagttatcaaggatgtgcgtaagaaaaagaaaaagagcgacaaaaccaaggaagcgctgaaagatgagctggaaatcgagttcgaggaacgtatgaaagacaagagcgcggcgctggagaagatggacaaagatgcgggcaaaaaggttaagaaccgtagcgaagacgagctgcgtaacgcggtgatgaacgatgcgtttaaacacctgatcgcgaaagacaaggatgagtacagcctgattgaacgttatcaggcgttcccggaaaacctggacgcgccgattagcgagaagagcctgatgtttctgtgcagctgcttcctgagccgtcgtgatatggagctgtttaaggcgcgtatcaccggtttcaaaggcaagatggttgaaggcgaggacagcctgaaatacatggcgacccactgggtgtacaactatctgaacttcaagggcctgaagcgtaagatcaacacccgttttgaaaaagagaacctgctgttccagattgttgatgaactgagcaaagtgccggactgcctgtaccgtgttatcaaagataagaacgagtttctgctggacattaacaagttctataaacaaaccaagggtgaagcggagagcccggaaaacgaggaagtggttaacccgatcattcgtaaacgttttgaggacaagttcaactactttgcgctgcgttatctggatgagttcgcgggttttgaaaacctgaagttccagatctacgcgggcaactatctgcaccacaaacaagaaaagaccagcgcgcagacccaactgaagaccgaccgtaaaatcaaggagaaaattaacgttttcggtaaactgagcgatgtgaacaaggcgaaagcgaacttctttgcgaacaaaaccgaggacagcgatatggacgaaggcctggaggaatacccgaacccgagctataacatcaacggtggcagcatcctgattcacctgaacctgaacaagtaccgttatggtcaggagttccacgagctgaaacaactgcgtatcgaaaaggagaaacgtggcgaaaacaaaaccgacaagattagcatcattaaggacctgttcgaggacaacaccgaaatcaaagaggaagattgggttttcccggtggcgctgctgagcctgaacgaactgccggcgctgctgtacgagatgctggttaacaaaaagagcagcaaggacatcgagcagatcattgcggaccgtatcgtgagccactacaaaaagattaaggatttcgagggcaccgcggatgaactgaaggacaaaaacctgccggttaacctgcgtaaggcgttcggcgcggacgataaaaacaccgacaagctggaaaacgcgatcaccaaagatattgaagcgggcgaggacaaactgcagctgattaaggagaacacccgtgaaatgcgtagcaacaaccgtaagtacgtgttttatctgaaggagaaaggcgaggaagcgacctggctggcgaaagacatcaagcgtttcatgccggaaaacgcgaaaaaccagtggaagagctacaaccacaacgagctgcaaaagggtctggcgtactatgaactggagcgtcagaacgttctggcgctgctggaaagcaaatgggatatggacagctgccacccgcactggggtgaggacctgaaggaactgtttattacccacagccgtttcgacgatttttacaaagcgtatatgctgtgccgtcagggcttcctggagcaatttaagaccctggttatccgtaacaaaagcgacaaaaagctgctgaacaaagttctgaaggatgtgttcatcccgtacaaaaagcgtttctttgtgattaacagcctggaaaacgagaaaaaggcgctgctgagccacccgattgttctgccgcgtggtctgtttgacaacaaaccgaccttcatcaagggcgtgagcctggaaaacgatccgagccgtttcgcgaactggtttgcgtacctgcgtcaggaagcgaagaacgatcaccaagttttctacgattttgaacgtgactatgtgaaagcgttcagcgagctgaaggacaaaagcaagtacaacaacaacaagcacttcaacttcaaggtggacagcgaaattcgtatgtgcctgcagaacgatctggttctgaagctgatcgtgaaaaagctgttcaaaggtatctttgatgttgacgaaaacattaagctgaacgacttctacctggaaaaaaccgaggtggcgaagcagcgtgagcaagcgctggatcaaaacaaacgtctgaagggtgacgatggcgatgttatctataaagaggaccacctgttccgtaaaacctttgcgaaggatttcctgaacggcaagctgcacttcgataaatttaagctgaaagactttggcaaagcgctggtgttcgcggcggacgaaaaggttaaaaccctggtgagctacagcgagaacgcgtggacccaggaagagctgcaaaaagaactgcacaccaataccgacagctatgagcgtattcgtcaggatgagttcttcaaaaagatccacgagctggaggaaagcatttggcagaagcacaaacacgaacgtgagaaactgcaagacaagagcggtaacgaaaactttaacaactacgtgaaagttggcgtgctggagaagctgaacgatagctttaaagacgagttcgagaacctgtataaggacaaaaagaacaagcgtatccagaagctgcgtcaatgcaaccacgtggttcagaaagcgtactgcctggttcaactgcgtaacaagttcagccacaaccagctgccgccgaaacaactgttcgactttatgaccgaaaccctggcggaaaaggataaacagacctacagccgttattttatggatgttaccgacaagatggtgcaagagttcaaaccgctggtgtag

SEQ ID NO: 37 - DNA, 3417 bp, Artificial Sequence
  Description of Artificial Sequence: Synthetic polynucleotide
atggaaacacaaattgtaaacaaaaaaagaaccttaaaagatgacccacagtactttggcacttatctaaatatggcaagacacaatattttcttaattgaaaatcatattgcacaaaagtttgaaaaaaataaattgggagttgttaaaagcgatgaacacattgcaagccgacagttttttgatgctgcttttaaaaataataaactagcaaatagcaaacagatttttaatgcctttactagatttattcatgttgctaaaattttcgataacgatttattgcctaaatcagaaaaacaagaagaaggctttcagcaagatagtatagacttcaacttgctatcagaaacctttttcagttgttttaaagagttaaatcaatttagaaacaacttctctcactattaccatatagaaaacgaagaaaaaagaaatctatttgtaagtgaaactttaaaatactttgtaattaaggcttatgagaaagcaattgcttatgctgaacaacgatttaaggacgtattcaagcacgaacattttaatatagcacgtaataaaaagttatttactcttcaccaagaatttactagagatggcttagtgtttttttgctgtctgtttttagagaaagaatatgcctttcattttatcaacaaaataattggttttaaagacacccgaaccgcagaatttaaagccactcgagaagtgttttctgttttctgtgttacattaccccacaatcgctttataagcgaagaccccgcacaagcttatattttagatgcgctaaactatttgcatcgttgcccaactgaactctacaataacttgagtgaagatgctaaaaagcattttcaacccacccttagttatgaggcagtacaaaatattcaaggcagcagcgttaataatgaacaacttcctattgaagattttgatgattatatacaaagcattaccacacaaaaaagaaataccgaccgcttcccattttttgccttaaaatatttagataataaagaaagttttaaacccctgtttcatctgcatttaggtaagctattattaaaatcttacaagaaaaatcttttaggcaatgaagaagaccgctttatagtagaaagctttaccacttttggtactcttgaaaactttcaattgagtaatatagaagaagaaaacaaagaagaaaaagtgcgtgaaataactcaacttaaaaaagagattacaatagaacaatacgcccctaaataccatatagctaacaataaaattgctttaaacctaagcaataataaatactacaacggaaattttctcagttttcatcccgaagtttttcttagcatacacgaattacctaaagtagcactcttagaacatttattgcccggtaaagccactcagcttattgaaaactttgtcaacttaaatagcagccatattttaaacagccaatttattgaagaagttaaatcaaaactcacttttacacgtccactaaaaaaacaatttcataaagataagcttactatttacaactatacacttcaacaactgaataataaaataaatgaaataatacagtttattgatgacaataaagaacacgctgatgatgaaacaaaaaaccaaataaaaaataaaaaatctgagttaaaaaatttgtattacaataggtatgtagttcaagttgtagatagaaaacaacaattagatgctatattaaaaacctataacctcaaccacaaacaaatacccgagcgcatcattaactattggctgcaaattaaagaggtaaaagatgatactactttaaaaaacaaaataaaagccgaaaaagaagaatgcaaacaacggcttaaagacttagctaatcttaaaggcccaaaaattggcgaaatggctactttcttagctaaagatattattcatctagtaatagacttacaagtaaaaaagaagattaccactttttattacgaccgcttgcaagaatgccttgccttatatgcagatattgaaaaacaacaaacctttaaaagaatatgtagcgaattaggtttgttagatgccttaaaaggacatccgtttttaaaccaaattattttaggtaattattctaaaaccaaagatttttatagagcctacttacaacaaaaaggcaccaataccattgaaaaatatgattataatagaaagaaaatcgtagaaagcaattggatgtacaccacattctacaatgtggaaaataaacaaactattatttccatacccaataataaaccagtgccttattcttacaaacaatggcaagcaccccaaaccgattttaataaatggctaagcaatacttcaaaaggcatagataagcaacagccaaaacccatagacttgcccaccaatttatttgatgaaacacttaattcagcccttcagcaaaaattacaaaacccattacccaacgaaaaagccaattatacagccttactgaaagcatggatgccccaaagccagccattttacaatatgccacgctcttatatggtatatgataatgaggtaaattttacgcccggtacacaagccacttataaaggctattttgaaaaaactatacaaaaagtattgaggcaaaaaaacgaacaaataaaaaaagacaatctaaaagcaataaagaaaaaacccttttacacggcaagccaaatattagcggtatgtaacaatgctattacagaaaatgaaaaactaattagattttacgaaaccaaagaccgcatattgttgctcattgttcaagaattaagcggcatgcaaatgtgcttgcaaaaaatggatataaaatcgcaacaaagccccctaaacgaaatcatagaaataaaagaagtaatacaccaaaaaaccattactgcacaacgcaaaagaaaagattataccatacttaaaaagttagaaaaagataaaaggctgcccaatttactgcaatactttgatgaagatactattccattcgacactatcaataaagaactatttcattataaccaaagccgtgaaaagatttttgatagcagttttcttttggaaaaaactatagtagaaaagctacagcaaaatcaaagcatgcacatactcactaccatgcaagaagaaaaaaataaaaaagaaggcacagacgtaaaaaatattcaattcgatatttacacccaatggctgcaagaaaataagttcattagccaaaccgaagccgattttttacttactgtgcgcaataaattttcacacaaccaatttcccgaaaaaataaaaatagaaaaagaagttacatttgatgaaaaccaaaataaagcaagccaaatatgtgaaaactaccataaaaaaatacaagcaatcattgcccaactaaactag

SEQ ID NO: 38 - DNA, 3417 bp, Artificial Sequence
  Description of Artificial Sequence: Synthetic polynucleotide
atggaaacccagatcgttaacaagaaacgtaccctgaaagacgatccgcaatacttcggcacctatctgaacatggcgcgtcacaacatctttctgattgagaaccacattgcgcagaaatttgaaaagaacaaactgggcgtggttaagagcgacgagcacatcgcgagccgtcagttctttgatgcggcgttcaaaaacaacaagctggcgaacagcaaacaaatcttcaacgcgtttacccgtttcatccacgtggcgaagatttttgacaacgatctgctgccgaaaagcgaaaagcaggaagagggttttcagcaagacagcattgatttcaacctgctgagcgaaaccttcttcagctgcttcaaggaactgaaccaatttcgtaacaacttcagccactactatcacatcgagaacgaggaaaaacgtaacctgtttgttagcgaaaccctgaagtacttcgtgatcaaagcgtatgagaaggcgattgcgtacgcggaacagcgttttaaagacgttttcaagcacgagcacttcaacatcgcgcgtaacaagaaactgtttaccctgcaccaagagttcacccgtgatggtctggtgttcttttgctgcctgtttctggaaaaagagtacgcgttccactttatcaacaaaatcattggctttaaggacacccgtaccgcggagttcaaggcgacccgtgaagtgtttagcgttttctgcgtgaccctgccgcacaaccgtttcatcagcgaggacccggcgcaggcgtatattctggatgcgctgaactacctgcaccgttgcccgaccgagctgtataacaacctgagcgaagacgcgaagaaacactttcagccgaccctgagctacgaagcggttcagaacattcaaggtagcagcgtgaacaacgagcaactgccgatcgaagattttgacgattacatccagagcattaccacccaaaaacgtaacaccgaccgtttcccgttctttgcgctgaagtatctggataacaaagagagctttaagccgctgttccacctgcacctgggtaaactgctgctgaagagctacaagaaaaacctgctgggcaacgaggaagaccgttttatcgttgagagctttaccaccttcggcaccctggaaaacttccagctgagcaacattgaggaagagaacaaagaagagaaagtgcgtgaaatcacccagctgaagaaagagatcaccattgaacaatacgcgccgaaatatcacatcgcgaacaacaagattgcgctgaacctgagcaacaacaaatactataacggtaactttctgagcttccacccggaagtgttcctgagcattcacgaactgccgaaagttgcgctgctggagcacctgctgccgggcaaggcgacccagctgatcgaaaactttgttaacctgaacagcagccacatcctgaacagccaattcattgaagaggtgaagagcaaactgacctttacccgtccgctgaagaaacagttccacaaggacaaactgaccatttacaactataccctgcagcaactgaacaacaaaatcaacgagatcattcagttcattgacgataacaaggagcacgcggacgatgaaaccaagaaccaaatcaagaacaagaaaagcgaactgaaaaacctgtactataaccgttacgtggttcaggtggttgaccgtaagcagcaactggatgcgatcctgaaaacctataacctgaaccacaagcagattccggagcgtatcattaactactggctgcaaatcaaagaagttaaggacgataccaccctgaagaacaaaattaaggcggagaaagaagagtgcaagcagcgtctgaaagacctggcgaacctgaaaggtccgaagatcggcgaaatggcgacctttctggcgaaagacatcattcacctggttatcgatctgcaggtgaagaaaaagattaccaccttctactatgaccgtctgcaagagtgcctggcgctgtacgcggacatcgaaaaacagcaaacctttaagcgtatttgcagcgagctgggtctgctggatgcgctgaagggccacccgtttctgaaccagatcattctgggtaactatagcaaaaccaaggacttctaccgtgcgtatctgcagcaaaaaggcaccaacaccatcgagaagtacgattacaaccgtaaaaagattgttgaaagcaactggatgtacaccaccttctataacgtggaaaacaaacagaccatcattagcatcccgaacaacaaaccggtgccgtacagctataagcagtggcaagcgccgcaaaccgatttcaacaagtggctgagcaacaccagcaagggtatcgataaacagcaaccgaagccgattgacctgccgaccaacctgtttgatgaaaccctgaacagcgcgctgcagcaaaaactgcagaacccgctgccgaacgaaaaagcgaactataccgcgctgctgaaggcgtggatgccgcagagccaaccgttctacaacatgccgcgtagctacatggtttatgacaacgaggtgaactttaccccgggcacccaggcgacctacaagggttatttcgagaaaaccattcaaaaggttctgcgtcagaaaaacgaacaaatcaaaaaggataacctgaaggcgattaaaaagaaaccgttctacaccgcgagccagatcctggcggtttgcaacaacgcgatcaccgaaaacgagaaactgatccgtttctacgaaaccaaggaccgtatcctgctgctgattgtgcaggaactgagcggtatgcagatgtgcctgcaaaaaatggacatcaagagccagcaaagcccgctgaacgaaatcattgagatcaaagaagtgattcaccagaagaccattaccgcgcaacgtaagcgtaaggactataccatcctgaagaaactggagaaagataagcgtctgccgaacctgctgcagtactttgacgaagataccatcccgttcgacaccattaacaaagagctgttccactataaccaaagccgtgaaaagatttttgatagcagcttcctgctggagaaaaccatcgttgaaaagctgcagcaaaaccagagcatgcacatcctgaccaccatgcaagaagagaaaaacaagaaagagggcaccgacgtgaagaacatccagttcgatatttacacccagtggctgcaagagaacaaatttatcagccaaaccgaagcggacttcctgctgaccgttcgtaacaagtttagccacaaccagttcccggaaaaaatcaagattgaaaaagaggtgacctttgatgagaaccagaacaaggcgagccaaatctgcgaaaactaccacaagaaaattcaggcgatcattgcgcaactgaactag

SEQ ID NO: 39 - DNA, 3282 bp, Artificial Sequence
  Description of Artificial Sequence: Synthetic polynucleotide
atggtaaatgtaaacaaaagaacactcaccggtgatccgcagtattttggcggatacctgaatttggcaaggctaaatgtatttgcgattagcaatcatattgccgaaaagataaatccatttttgaagaagggaaaggttggagtattacaggatgacgaaaatattcccgatagttttatttgcaataaaattaaggagaagccgaatctcttttatacacagcttgtaaggttttttccgattgcgcgagtttatgattcggatagattgccaaaggaagaaaaattattaacaaagtgcgagggtatagattattccctgcttacaggggatatgaaaatttgtttttcggagttgaatgatttcaggaatgattattcgcattacttttctattaaaaccgggacggataggaaagttgaaataagtgaaagactttcggattttttaatgactaattatcttagggctatagaatatacaaaggttaggtttaaagatgtttataatgattcacattttcaaattgcctcaaagagaatattagttgacgaaaataatattataacacaggatggattagttttctttatgtgcatatttcttgaaagagaaagtgcttttcattttataaataaaataattggtttcaaagatacgaggtctttggatttcaaagcgatgagggaagttttttctgctttttgtattacgcttccgcacgataagtttataagtgatgatggtaagcaggcttttatacttgatttgctgaatgaactgaataggtgtccgaaggaattgtttgagaatatttcaagcgaagagaagaagcaatttcagccgaatgtgagcgagagtgcagcggatattgaagagaacagtattccggctgatttacctgaagaagattttgaagaatatattcaaagtataataagcaagaaaagaaagacggacaggtttccgtatttcgcagtaaagtatcttgatgaaaaaacgaatattaattttcatttgaatctggggaagatagaacttgttactcgcaagaagaaatttttaggaggagaagaggatagagatattattgaggatgcaaaggtgtttgggaagctgggagaatacgctgatgaaagagcggtttcgaaaagacttggtatggagtttcagttattcaatccgcattatcagattgagaataataaaattggattttcttttagcccaatagaatgttctataaaaaatgttaatggtaagccgaatttgaaattaaatccaccgaatgcatttttaagtattaatgaaatgccgaaagtagttcttctggagattttacagagaggaaaagtaacggagattataaaggaattcattcaagcaagcacggataaaatactgaatagagaatttattgaggaagtaaagagtaaattggattttaaaaaaccatttaacaggagttttagcaagaaaaggaattctgcttatggacctaaaggactgcaaatattaaccgaaagaagaacttctctaaatttaattttaaaagaacataatctgaatgacaaacagatacccggaagaatattggattactggatgaatattgttgatgtgacggatgataaggcaatagccaatagaattcaggcgatgaaaaaggattgcagagacaggcttaaacaaaaagctaaaaacaaagcaccaaagattggagagatggcaacgtttcttgcaagagatattgtagatatggtgattgatgaaaatgtaaagaaaaagataacatcattttactatgataagatgcaggaatgcctggcgctttacggagatgcagaaaagaaggagttgtttataaggatttgcggagaggaattaaatctttttgataagggaataggacatccgtttttatttgagcttaatttgcaaagtataaataagacatcggaattgtatgagaaatatttgattaaaaaaggaacggctgagcatattaaatggaatgaaaggacaaagaagaattataaagttgaaacatcgtggctatatacaaatttttataacaagatttggaatgaagagaaaaagaaaatggaaacgaagctaaaacttcctgaggatttatcaaaattaccgttttcgattcgcaaccttactaaagaaaagtcttcgcttgataaatggctaaacaatgtgacgaaaggatgcttagaaaaagataggacgaagccaattgatttgccgacaaacatatttgatgaaacattagttaagataataagagaaaaactaaatgataaacaagtatcgtataaggatacggataaatattcaaaattgctggagttatggaagggtggagatacacagccgttttacaatgcggagcgagaatacactgtttatgaagagaaggtgcgatttagattgggtgaaaaaaattcatttaaagaatattttaaggatgctttagagaaagtttttaaaaaagaatcttcaaaaaggcagagcgaacgagggaagccaccgatacaaaagaaagatttgctgacggtttttaacgatgccataacagaaaacgaaaaggtggtgcgtttttatcagacgaaggatagggtgatgctgatgatggtaaaggatttaatgggagcggaacttgattttaaattaagtgaaatatatcctttgtcggaaaagagtccgctaaacatagaggaagaaatagagcaaagagtggaggggaaattaagttatgacggggatggaaattatataaaagggggtaaggagagtattacgaaaataatttatgccagaaggaagagaaaagatttcacagtgtttaagaaacttacgtttgataagcgattgccggaattgtttgagtattatgcagaagagagaataccatacgaaaaacttaaggcagaattggacgaatacaacaaacacagggatatggtatttgacgtggtatttgaactggaaaagaagataatggataagccggaagctttgagggaaatggaggatgtgggggataaaaatgtgcgacataaaccatatttgaactggttgaaaaaaaggaaagtgatagataaaaagcagtatgcattattaaatgcgataaggaattcattttcgcataatcagtatccgccgagaatgatagtggaaaataaaattaagataaaagcgggaggaataacaccccaaatatttgaaagatataaagaagaaatagagataataatgaataaaatatag

SEQ ID NO: 40 - DNA, 3282 bp, Artificial Sequence
  Description of Artificial Sequence: Synthetic polynucleotide
atggtgaacgttaacaaacgtaccctgaccggtgacccgcagtactttggtggctatctgaacctggcgcgtctgaacgtgttcgcgatcagcaaccacattgcggagaagatcaacccgttcctgaagaaaggtaaagtgggcgttctgcaggacgatgaaaacattccggatagctttatttgcaacaaaatcaaggagaaaccgaacctgttctacacccaactggtgcgtttctttccgatcgcgcgtgtttatgacagcgatcgtctgccgaaagaggaaaagctgctgaccaaatgcgagggtattgactatagcctgctgaccggcgatatgaagatctgctttagcgaactgaacgacttccgtaacgattacagccactatttcagcattaagaccggcaccgaccgtaaagttgaaatcagcgagcgtctgagcgattttctgatgaccaactacctgcgtgcgatcgagtataccaaagtgcgttttaaggacgtttacaacgatagccacttccagattgcgagcaagcgtatcctggtggacgaaaacaacatcattacccaagatggtctggttttctttatgtgcattttcctggaacgtgagagcgcgttccactttatcaacaagatcattggctttaaagacacccgtagcctggatttcaaagcgatgcgtgaggtgttcagcgcgttttgcattaccctgccgcacgacaagtttatcagcgacgatggcaaacaggcgttcattctggatctgctgaacgagctgaaccgttgcccgaaggaactgtttgagaacatcagcagcgaggaaaagaaacagttccaaccgaacgttagcgaaagcgcggcggacattgaggaaaacagcatcccggcggacctgccggaggaagatttcgaggaatacatccaaagcatcattagcaagaaacgtaaaaccgaccgtttcccgtactttgcggtgaagtatctggatgaaaagaccaacatcaacttccacctgaacctgggcaagatcgagctggttacccgtaagaaaaagttcctgggtggcgaggaagaccgtgacatcattgaggacgcgaaagtgtttggcaagctgggcgaatatgcggatgagcgtgcggttagcaaacgtctgggcatggagttccagctgtttaacccgcactaccaaattgagaacaacaaaatcggtttcagctttagcccgattgaatgcagcatcaagaacgtgaacggcaaaccgaacctgaagctgaacccgccgaacgcgttcctgagcattaacgaaatgccgaaagtggttctgctggagatcctgcagcgtggcaaggtgaccgaaatcattaaagagtttatccaagcgagcaccgacaagattctgaaccgtgagttcatcgaggaagttaagagcaaactggatttcaaaaagccgtttaaccgtagcttcagcaaaaagcgtaacagcgcgtatggtccgaagggcctgcagattctgaccgaacgtcgtaccagcctgaacctgatcctgaaggagcacaacctgaacgacaaacaaattccgggtcgtatcctggactactggatgaacatcgtggatgttaccgacgataaagcgattgcgaaccgtatccaggcgatgaaaaaggactgccgtgatcgtctgaagcaaaaagcgaagaacaaagcgccgaagattggcgaaatggcgaccttcctggcgcgtgacattgtggatatggttatcgacgagaacgttaaaaagaaaatcaccagcttttactacgacaaaatgcaggaatgcctggcgctgtatggtgatgcggaaaagaaagagctgtttatccgtatttgcggcgaggaactgaacctgttcgataagggtattggccacccgttcctgtttgagctgaacctgcaaagcatcaacaagaccagcgaactgtacgagaaatatctgatcaagaagggcaccgcggaacacatcaagtggaacgagcgtaccaagaaaaactacaaagtggaaaccagctggctgtacaccaacttctacaacaagatctggaacgaggaaaagaaaaagatggaaaccaagctgaaactgccggaggacctgagcaagctgccgtttagcatccgtaacctgaccaaggagaaaagcagcctggataagtggctgaacaacgttaccaaaggctgcctggaaaaagaccgtaccaagccgattgatctgccgaccaacatcttcgacgaaaccctggtgaaaatcattcgtgagaaactgaacgataagcaggttagctacaaagacaccgataaatatagcaagctgctggagctgtggaagggtggcgacacccaaccgttctataacgcggagcgtgagtacaccgtgtatgaggaaaaagttcgttttcgtctgggcgagaagaacagctttaaggagtacttcaaagatgcgctggaaaaggtgttcaaaaaggagagcagcaagcgtcagagcgaacgtggcaaaccgccgattcagaagaaggacctgctgaccgtttttaacgatgcgatcaccgaaaacgagaaggtggttcgtttctatcagaccaaagaccgtgtgatgctgatgatggttaaggacctgatgggtgcggagctggatttcaaactgagcgaaatctacccgctgagcgagaagagcccgctgaacattgaggaagagatcgaacaacgtgtggagggcaaactgagctacgacggtgatggcaactatattaaaggtggcaaggaaagcatcaccaagatcatttacgcgcgtcgtaagcgtaaagacttcaccgtttttaaaaagctgacctttgataaacgtctgccggaactgttcgagtactatgcggaagagcgtatcccgtacgagaagctgaaagcggaactggacgagtataacaaacaccgtgacatggtgtttgatgtggttttcgaactggagaaaaagatcatggataagccggaagcgctgcgtgaaatggaggacgtgggtgataagaacgttcgtcacaaaccgtacctgaactggctgaaaaagcgtaaagtgattgacaaaaagcagtatgcgctgctgaacgcgatccgtaacagcttcagccacaaccaatacccgccgcgtatgatcgttgagaacaagatcaagatcaaggcgggtggcattaccccgcagatctttgaacgttacaaggaagagattgagatcattatgaacaaaatctag

SEQ ID NO: 41 - DNA, 3711 bp, Artificial Sequence
  Description of Artificial Sequence: Synthetic polynucleotide
atgcggatcatacggccctacggcaccagcgcgaccgagccggacgcgcaggacccggccaagcgccggcgcacgctgcggcgcaagctcgacgcgccgggcgcgacaacggtcaccgagcgcgacctcggagcgttcgcccgccgccacgacgtgctggtcatcggccagtggatctcgacgatcgacaagatcgccagcaagcccgcaggcttcaagaagcccggcgccgagcagcgggcgctgcggcgcaggctcggcgaggccgcctggcgccacatcgtggcacacggcctcctgcccgggcgcgccgagaccccctcgctcgaaaccctgtggtggatgcggctcgagccctatccgacgggcgatgccaagtacgggcgcgatcccaaaggacgctggtacgcgcgcttcgtcggcgagatcgagcccgaggagatcgacgccgatgcggtcgtcgagcgcatcgccgagcacctctacgcgcacgagcacccgatccacccgggcctgccgacgcgccgcgagggacggatcgcgcatcgcgccgcctcgatccaggctgccgtgccgaaggcggaacctcgtgccgcgcgcgcgacgtggacggatgcgcactggacgatctacgccgaggccggggacgtggcggcggtgatccgtgcggcggccgaagaggtccaggcgccgcccccgcccgacgacaaggcggcgaagggcaagcggcgctgggtcgggcccgacgtcgccggcaaggcgctgttcgagcactggcagcgcgtgttcgtcgatcccgagaccgaggccgtcttgagcgtgggcgaggtcaaggcgcggatcgagaacggcgacgaccgcctgcgggcgctgttcgagctccacgaagaggtccgcggcgcctaccgccggctcctcaagcgtcaccgcaaagccgtgcgcggatcctccggtaagccgacccggaccagcgatgtcgcccgtctcctaccgtcgtcgatggacgcactccagagactgcttgcggcgcagcgcgacaaccgcgacgtcaacgccctgatccggttcggcaaggtcatccactacgaggcggccgagccgacctccgaggttccgccggacgacgacgggcgaccgcgccacgacgagcccgcgcacgtgctcgacgactggcccgacgccgcgcgggtggcccggagccgcttctggaccagcgacggccaggccgagatcaaggccaacgaggccttcgtgcgcatctggcgtcgggtgctcgcgctcatgcaccgcacggcgacggactgggcgatgcccgaggccgatgacgatttcaccatggcgcgcgtgctcgagcgggccgttggcgaagacttcgaccaggcgcggcatcggcgcaaggtcgagctcctgttcggtgcacgagccgacctgttccggggtgacggcgccgacgacgcgctcgatcgcgaggtgctgcggttcgccctcgagcacctgcgcagcttgcgcaacaagtcctttcacttcgtcggcgtcggcggtttcaaggcagtgttgaccggggccaacgaggcgccggccgacggggctgcgccggcacaggcccgggccctctgggcgcaggatcagcgcgagcgggccaaacagctcggcaaggtcctgcagggcgtgcaggcgggggactacctcgagggcaacgagcttcgagcgctcttcgatgacctcgtcgcggcgatgacgacgccttccgacctgccgctgccccgcttcaagcgggtgctgctccgcgccgagaacatccgcgacaagcgccaagacgacccgcacctgcccgcgcccgccaaccgtctcgacctcgaggagccagcgcgcctctgtcagtacaccgcgctcaagctcgtctacgaacgaccgttccgccgctggctcgccgatgccgacgcggccaaggtccgaggctatgtcgagggcgccgcccggcgttcgaccgacgcggcgcgcaagctcaacgaccccaaggacgaggcgaaacgcgagcgcgtccgctcgaaggccgagcggatcgcgaacctggcgcccgacgcgaccatgcgcgatttcgtcaggacgctgatgcgtgagacggcgagcgagatgcgcgtgcagcgcggctacgagagcgacgccgagaacgcccgcgaccaggcgcgctacatcgaggacctcctgcgcgacgtcgtggcgctggcgttcctcgactacttccgggacgcgaagttcggattcctgctcgagattgccgcggaccgcacggtcgatccggcgaagcggctcgatccgaccacgctcgaagcccccgaggccgacgtgtcggcagaaccctggcaggtggcgctctatttcgtgagccatctcgcaccggtcgacgacatcgcgctcctcctgcaccagctgcgcaagttcgacatcctcgccgagaagcgcggtgcgggcaccgacgacgcgttgcgcgctcaggtcgaggccgtcatcaaggtcttcgatctctacctcgacatgcacgacgccaagttcgagggcggacgcgggctcgccggtctggaggacttcgcccaactcttcgagagccgcgagctcttcgaggagctggtcgcgaagccggtgggccaggacgacagcgaacgcgtgccggtgcgcggcctgcgcgagatcgcccgctacgggcatctgccgccgctcctgcccatcttccagaagcgcaggatcaccgaggaggatgcccgggagtttcgcgagcgcggaggcacgatcgcggaccggcagaaggagcgccaggcgctgcacgcggaatgggcggaaaagccgaaagcattcgctaaccactcggtggcggaatacacccgcgccctgcgagacgtcgcgcagcaccgtcattgcgccaatcacgtgagtctcacggcccatgtgcgcctgcatcggctgctgatgggcgtgctcggacgactgttggacttctcgggcctgttcgagcgcgacctctacttcgccgccttggcgctcgttcacgagaacggcttgaggacggaggaggcgttcggcaagcgttgcgcctatctgattggacagggacggatccttgctgcgatccgacatttggatgcggagattcaaaaagaactcggcggcctgtttcttttggacggcgccacaaaggtcatccggaaccacttcgcccacttcaaaatgctgcaaccttcgagggccgacgcggcggcgctcaacctgacgagcgaggtcaacggctgccggcagctgatgcgttacgaccgcaagctcaagaacgcggtgacgaaagccgtcatcgagttcttggaacgcgaggggctcgacatccggtggacctggaacgacgcgcacgagctgagcgtgccgacgctcaagacccgcgccgccaagcacctcggcggcagagccatcgccgaacgccgtgaggacggcgccgtgcccgacgtgagggatggatttccgatccaggaggcgctccacgccgctggctacgtcgagatgacagccgccctgttcgccggccatgcggcgcccatccgcaacgagatctgcgcgctggatctcgagcgcatcgactggcgccggccgcagcgcagggacggctccaaggggaaggggaaagggaaaggcaagaaccggcaccctgcgccgaataaggcccagtag

SEQ ID NO: 42 - DNA, 3711 bp, Artificial Sequence
  Description of Artificial Sequence: Synthetic polynucleotide
atgcgtatcattcgtccgtacggcaccagcgcgaccgagccggatgcgcaggacccggcgaaacgtcgtcgtaccctgcgtcgtaagctggatgcgccgggtgcgaccaccgttaccgaacgtgacctgggtgcgttcgcgcgtcgtcacgatgtgctggttattggccagtggatcagcaccattgataaaatcgcgagcaagccggcgggttttaaaaagccgggtgcggagcaacgtgcgctgcgtcgtcgtctgggtgaagcggcgtggcgtcatattgttgcgcacggtctgctgccgggtcgtgcggaaaccccgagcctggaaaccctgtggtggatgcgtctggagccgtacccgaccggtgacgcgaaatatggccgtgatccgaagggtcgttggtacgcgcgtttcgtgggcgagattgaaccggaggaaatcgacgcggatgcggtggttgagcgtattgcggaacacctgtatgcgcacgaacatccgattcatccgggcctgccgacccgtcgtgaaggtcgtattgcgcatcgtgcggcgagcatccaggcggcggttccgaaagcggagccgcgtgcggcgcgtgcgacctggaccgacgcgcactggaccatttacgcggaagcgggcgatgttgcggcggttatccgtgctgcggcggaggaagtgcaagctccgccgccgccggatgataaagcggcgaagggcaagcgtcgttgggtgggtccggatgttgcgggcaaggcgctgttcgagcactggcaacgtgtgtttgttgatccggaaaccgaagcggtgctgagcgttggcgaggtgaaggcgcgtatcgaaaacggtgacgatcgtctgcgtgcgctgttcgaactgcacgaggaagttcgtggtgcgtaccgtcgtctgctgaaacgtcaccgcaaggcggtgcgtggtagcagcggcaaaccgacccgtaccagcgacgttgcgcgtctgctgccgagcagcatggatgcgctgcagcgtctgctggcggcgcaacgtgacaaccgtgatgtgaacgcgctgattcgttttggcaaggttatccactatgaagcggcggaaccgaccagcgaggtgccgccggatgatgatggtcgtccgcgtcatgatgaaccggcgcatgtgctggatgactggccggatgcggcgcgtgttgcgcgtagccgtttctggaccagcgatggtcaggcggagattaaagcgaacgaagcgtttgtgcgtatctggcgtcgtgttctggcgctgatgcaccgtaccgcgaccgattgggcgatgccggaggcggatgacgatttcacgatggcgcgtgtgctggagcgtgcggttggtgaagactttgatcaagcgcgtcaccgtcgtaaggttgaactgctgttcggtgcgcgtgcggacctgtttcgtggtgatggtgcggatgatgcgctggaccgtgaggtgctgcgtttcgcgctggaacacctgcgtagcctgcgtaacaagagcttccactttgtgggtgttggtggctttaaggcggtgctgaccggcgcgaacgaggcgccggcggatggtgcggcgccggcgcaagcgcgtgcgctgtgggcgcaggatcaacgtgaacgtgcgaaacaactgggcaaggtgctgcagggtgttcaagcgggcgactacctggagggtaacgaactgcgtgcgctgttcgacgatctggttgcggcgatgaccaccccgagcgatctgccgctgccgcgttttaaacgtgttctgctgcgtgcggagaacattcgtgacaagcgtcaagatgatccgcacctgccggcgccggcgaaccgtctggatctggaggaaccggcgcgtctgtgccaatacaccgcgctgaaactggtttatgagcgtccgtttcgtcgttggctggcggatgcggatgcggcgaaagtgcgtggttatgttgagggtgcggcgcgtcgtagcaccgatgcggcgcgtaaactgaacgacccgaaagatgaggcgaagcgtgaacgtgtgcgtagcaaggcggaacgtattgcgaacctggcgccggatgcgaccatgcgtgattttgtgcgtaccctgatgcgtgaaaccgcgagcgaaatgcgtgttcagcgtggctacgagagcgacgcggaaaacgcgcgtgatcaagcgcgttatattgaggacctgctgcgtgatgtggttgcgctggcgttcctggactactttcgtgatgcgaaattcggttttctgctggaaattgcggcggaccgtaccgtggacccggcgaaacgtctggacccgaccaccctggaggcgccggaagcggatgtgagcgcggagccgtggcaggtggcgctgtatttcgttagccacctggcgccggtggacgatattgcgctgctgctgcaccaactgcgtaaatttgacatcctggcggagaagcgtggtgcgggcaccgatgatgcgctgcgtgcgcaggttgaagcggtgatcaaagttttcgacctgtacctggacatgcacgatgcgaagtttgagggtggccgtggtctggcgggcctggaagatttcgcgcagctgtttgagagccgtgaactgttcgaggaactggtggcgaaaccggttggtcaagacgatagcgagcgtgtgccggttcgtggcctgcgtgaaattgcgcgttatggtcacctgccgccgctgctgccgattttccagaaacgtcgtatcaccgaggaagacgcgcgtgagtttcgtgaacgtggtggcaccatcgcggatcgtcagaaagagcgtcaagcgctgcatgcggagtgggcggaaaagccgaaagcgttcgcgaaccacagcgtggcggaatacacccgtgcgctgcgtgacgttgcgcaacaccgtcattgcgcgaaccatgtgagcctgaccgcgcacgttcgtctgcaccgtctgctgatgggtgttctgggccgtctgctggacttcagcggcctgtttgagcgtgatctgtactttgcggcgctggcgctggtgcatgaaaacggcctgcgtaccgaggaagcgtttggtaaacgttgcgcgtatctgattggtcagggccgtattctggcggcgatccgtcacctggacgcggagatccaaaaggaactgggtggcctgttcctgctggatggtgcgaccaaagttatccgtaaccacttcgcgcactttaagatgctgcagccgagccgtgcggatgctgcggcgctgaacctgaccagcgaggtgaacggctgccgtcaactgatgcgttacgatcgtaagctgaaaaacgcggtgaccaaagcggttattgagtttctggagcgtgaaggtctggacatccgttggacctggaacgatgcgcacgaactgagcgttccgaccctgaaaacccgtgcggcgaaacatctgggtggccgtgcgattgcggagcgtcgtgaagatggtgcggtgccggacgttcgtgatggttttccgatccaggaagcgctgcatgcggcgggctatgtggaaatgaccgcggcgctgtttgcgggtcatgcggcgccgattcgtaacgagatctgcgcgctggacctggaacgtatcgattggcgtcgtccgcagcgtcgtgacggtagcaagggtaaaggcaagggtaaaggcaagaaccgtcacccggcgccgaacaaggcgcaatag

SEQ ID NO: 43 - DNA, 3279 bp, Artificial Sequence
  Description of Artificial Sequence: Synthetic polynucleotide
atgcaaaagcatcaaataatggataaaggcaatgcagagggcaattaccggcactttgatgaagaagccgataaacctttttatgctgcttacctgaatacggccaaacaaaacatctttttagtgctcagggacatttctgaaaagctggacctgggtttcaatttcgacagtgatgatcagctatttagtgtggagctgtggaaacagcttaaaaccgggaaaaggcctaatcttacccagaagatcatagcgcatttaaaacagcaattgccgtttttagaaattgcagcaattgctaatgcccgtaaacaatccaatgaccataaagcccaacctcaaccggaggactactatcacattttagagcattgggtcagccaattgcttgattactgcaattactacacccatgccacacacaattcggtcaatatggctcgtgtgatcattggaggaatgcttgatgtatttgattcggctcgcagacgtgtgaaagaccgtttttccttaatgcccgcagatgtagagcatttggttaggcttgggccaaagggcgggcaaaatgatcgttttcattacagtttcctggataagcaagggcgcctaaccgaaaaaggatttttattctttacatctctttggcttaaaaaaaaggatgcccaggaatttttgaaaaaacatgaaggatttaagcaaagccaggaaaacgctgataaagctactttagaagccttcacgattttcggtataaagttacccaagccacgattaacaagcgatctgggtgatcagggcttattcatggatatggtgaatgagcttaaacgttgtccggaagagctttattcactgcttagcaaagaagaccaagccacatttaaaccgcatgattctgaagaagcaacaaatgatgatgaaaacccacctgaattaaagcgaaatcagaaccggttttactactttgccttgcgatacctggaaaatgcctttcagaacctcaggtttcaaattgatctgggcaattattgcttcaaaacttatgagcaagagatagagcaggtagcgtacaaaagacggtggtttaaacgaataaccgcttttggacggttgacagattacaaggagcataaccagccaatggaatgggaagaaaaattgctaaaagttcctgatagggacaaacccgacacctatatcactgataccacaccgcattaccatttaaatgaaaacaacatcgggcttaaaaaagtaacggataaggataaagtttggccagaaattcccaaaaaagaaaatggtaaaaaaccggaaggtaatcctcccgatttttggttaagtatttacgagctgccggcagtagttttttatcaaatcctttatgaaaaaggcttagcacagttttcagccgaaagcataatcgaaatatacgccggagaaattcaaaaattgctggatgacgtaaaagtcggaaacattgcttccggatattcaaaggagcaattgcaaacagaactggaaaaccgggctttgcacatttcttatatacccaaaccggtgatcaaataccttttgggagaggatgaatggtcatttgaagaaaaagcggctgcccgcctgcaggcgttaaaggctgaaaacgaccaattgctaaaaaaagtaaagcgaaagcagctccactttaggcaaaaacccagcaacaaagattttaggatcatgaaaccagaggaaatagcggatttcctggcccgcgacatgatctggctgcaacaacctgataataaggaaaaaaacaaacccaataagacagaatttcatcatcttcaaggcaaacttacttatttcaggaagtacaaaatgactttactgaaaacattcaggcgctgtaacctggtggatgccccaaatgcacacccttttcttaaccaaatcaatttattggcctgcaaaggcctcctgaacttttatgtaacctacctggagcacaggaaggctttcctggagcaatgtaccaaagaacaggattatgcagcctatcactttttaaaggtaaagagggataaggatgctattgctacattgatcgaaaaacagcaggatgccgtttgcaacctgccaagagggttgttcaagcaacccatcatggaggcattaaaaaattcggatgaaacccgtgggttagcagcatcactcgaaaaaatggatagggccaatgtggccttcattattcaaaattactttcatgaagtccagcaagatgacaaccaggcgttttacgactacaaaaggagttatgaattacttaataagctatatgaccagcggaaaacaaacgacagaagccccttgccatcagtctttttttcaacccgggagctggaggagaaaaaagacgagatcccgcaaaaattagcagataaggtgcaatcacggattgaaaaaaacagtattaaagacgaaaaagaaaaggaacgaattcagcaaaaatacaggaagcgatacaagcaattcactgaaaatgaaaagcaaatccggttttttaaaacctgtgacatggtcctgtttttaatggcggaccaaatgtaccgcagtggagacccaatcggattgcatgataataacgataatacggcccagggaataacaggtatgggggaagcatacaagctcaagaacatcagacccgatgcagaaaggagtattctgtcacatgaaacccttgttaaaattccggtttattttaataatgcaagtgaaagccgctccaaaaccattgtaagggagagaatgaaaattaaaaattacggggatttccgtgctttcctgaaagatagaaggctaaccggtttgttgccttacattgaggcagatgaaatagtatatgaggctttgaaaacagaatttgaggcttttcatgatgcgcggattgaggtttttgaaaaaatcctcgaatttgaaaaaatatttcttataaaggttagacctaaagcaaaaaagaagaggtatatacctcatgaattactgcttcaacaaaacgcgatagatttgccgtcttatcaaataaagaacatgatcgctttacaccattcttttaatcacaaccaatacccggatgctaaacaatttggtgaatacatagacggaagcaattttaaccagttaaaattgtacactgctgataaccaggaagtaatggcccattccatcattgtgcaattaaaaaaactggcgttatggtactatgataaagccataaaactgacaaatgcttcttag

SEQ ID NO: 44 - DNA, 3279 bp, Artificial Sequence
  Description of Artificial Sequence: Synthetic polynucleotide
atgcagaaacaccaaatcatggataagggtaacgcggagggcaactaccgtcacttcgacgaggaagcggataaaccgttttacgcggcgtatctgaacaccgcgaagcagaacatctttctggtgctgcgtgacattagcgagaaactggatctgggtttcaactttgacagcgacgatcagctgttcagcgttgaactgtggaaacaactgaagaccggcaaacgtccgaacctgacccagaaaatcattgcgcacctgaagcagcaactgccgttcctggaaatcgcggcgattgcgaacgcgcgtaaacagagcaacgatcacaaggcgcagccgcaaccggaggactactatcacatcctggaacactgggtgagccaactgctggactactgcaactactatacccacgcgacccacaacagcgtgaacatggcgcgtgttatcattggtggcatgctggacgtgttcgatagcgcgcgtcgtcgtgttaaagatcgttttagcctgatgccggcggatgtggagcacctggttcgtctgggtccgaagggtggccagaacgatcgtttccactacagctttctggacaaacaaggtcgtctgaccgaaaagggcttcctgttctttaccagcctgtggctgaagaaaaaggatgcgcaggagttcctgaaaaagcacgaaggttttaaacagagccaagagaacgcggacaaggcgaccctggaagcgttcaccatctttggcattaagctgccgaaaccgcgtctgaccagcgacctgggtgatcaaggcctgtttatggacatggttaacgaactgaagcgttgcccggaggaactgtacagcctgctgagcaaagaggatcaggcgaccttcaagccgcacgacagcgaggaagcgaccaacgacgatgagaacccgccggaactgaaacgtaaccaaaaccgtttctactattttgcgctgcgttatctggagaacgcgttccagaacctgcgttttcaaatcgatctgggtaactactgcttcaagacctatgagcaggaaatcgagcaagtggcgtacaaacgtcgttggttcaagcgtattaccgcgtttggccgtctgaccgactataaagagcacaaccagccgatggaatgggaggaaaagctgctgaaagttccggaccgtgataagccggacacctacatcaccgataccaccccgcactatcacctgaacgagaacaacattggtctgaaaaaggtgaccgacaaggataaagtttggccggagatcccgaaaaaggaaaacggtaaaaagccggagggtaacccgccggacttctggctgagcatctacgaactgccggcggtggtgttctaccagattctgtatgagaaaggtctggcgcaattcagcgcggagagcatcattgaaatctacgcgggcgagattcagaaactgctggacgatgtgaaggttggtaacatcgcgagcggctatagcaaggaacagctgcaaaccgaactggagaaccgtgcgctgcacatcagctacattccgaaaccggtgattaagtatctgctgggcgaagatgagtggagctttgaggaaaaagctgcggcgcgtctgcaggcgctgaaggcggagaacgaccaactgctgaaaaaggttaagcgtaaacagctgcacttccgtcaaaaaccgagcaacaaggattttcgtatcatgaaaccggaggaaattgcggacttcctggcgcgtgatatgatctggctgcagcaaccggacaacaaggagaaaaacaagccgaacaaaaccgagttccaccacctgcagggcaagctgacctactttcgtaaatataagatgaccctgctgaaaacctttcgtcgttgcaacctggtggatgcgccgaacgcgcacccgttcctgaaccaaattaacctgctggcgtgcaagggcctgctgaacttctacgttacctatctggagcaccgtaaagcgtttctggagcagtgcaccaaggaacaagattacgcggcgtatcactttctgaaagtgaagcgtgacaaagatgcgatcgcgaccctgattgaaaagcagcaagacgcggtttgcaacctgccgcgtggtctgttcaaacagccgatcatggaggcgctgaagaacagcgatgaaacccgtggcctggcggcgagcctggaaaaaatggaccgtgcgaacgtggcgttcatcattcagaactactttcacgaggttcagcaagacgataaccaagcgttctacgactataagcgtagctacgaactgctgaacaaactgtatgatcagcgtaagaccaacgaccgtagcccgctgccgagcgtgttctttagcacccgtgagctggaggagaagaaggacgaaatcccgcagaaactggcggacaaggttcaaagccgtatcgagaaaaacagcattaaggatgaaaaagagaaggaacgtatccagcaaaagtaccgtaaacgttataagcagtttaccgagaacgaaaagcaaatccgtttctttaagacctgcgacatggtgctgttcctgatggcggatcagatgtaccgtagcggtgacccgatcggcctgcacgacaacaacgataacaccgcgcaaggtattaccggtatgggcgaagcgtataaactgaagaacatccgtccggatgcggagcgtagcattctgagccacgaaaccctggtgaaaatcccggtttacttcaacaacgcgagcgagagccgtagcaagaccatcgtgcgtgaacgtatgaagatcaagaactacggtgatttccgtgcgtttctgaaagaccgtcgtctgaccggcctgctgccgtacatcgaggcggatgaaattgtttatgaggcgctgaagaccgagttcgaagcgtttcacgacgcgcgtatcgaggtgtttgaaaaaattctggagttcgaaaagatctttctgattaaagttcgtccgaaggcgaaaaagaaacgttacatcccgcacgaactgctgctgcagcaaaacgcgattgacctgccgagctatcagatcaagaacatgattgcgctgcaccacagcttcaaccacaaccagtacccggatgcgaaacaattcggcgagtatatcgacggcagcaactttaaccagctgaagctgtacaccgcggataaccaagaagtgatggcgcacagcatcattgttcagctgaagaaactggcgctgtggtactatgacaaagcgattaagctgaccaacgcgagctag

SEQ ID NO: 45 - DNA, 3162 bp, Artificial Sequence
  Description of Artificial Sequence: Synthetic polynucleotide
atgactttaccagataaacaacaatccacaatatattcaatggacagatcagaagataaatatttttttgccctgtatttgaatattgcacagaataatgtggataaagttcttaaagaatttgacagttggtttaatagcctgaatgaaacaagccagggaaaatataatagtgcacaggccaaatggcttgataacagattaccgggttctgattcagatgttcttgaagccaaagaaagacttgtgtatttacgcaggttttttccttttattgaaactgaatttacaacgaaagaatatcatggatacagggaaaaactcttgatgttatttgaaagattgaatgacttcagaaatttctttacacatgttcattacgaaaggaatgaacttgaattttccaggaataaaaaaatgtttgagttcttaaatgaagtcaaagaaattgccttaaataaattaaatcagcatccctattatttagatgataatattttaaatcatctgcatgatcctgatcagaggtttaattttcaaaaagaaaacaatataaaagatgcaataaacttttttgtttgtttgtttctcgaaaacaaacatgcacatgaatatcttaaaaagcaaaagggatataaaagttctcataatcctgagcacagagcaacactgaagacgtatactttttatagcataaaattgcctcgtcctgtatttgaaagcagagacatgaagcttaggcttatccttgatgcattgaatgaactgaaaaaatgtcctaaacaattatacgatcatttatcggaaaaacaccaaaagctttgccaggttgaatctgtaaaacaaaaagaaaatgaggaatctggagaaacagaagaaattaaggagtatataccctttattcgacatgaagataagtttccttattatgctcttcgattcattgatgacctggaattactcaaagatattcgttttaaaatcaaacggggattgggaaaagaattttttcacactcatgaaactgcaactcaaccggttgttagaaataaaaaagtctttactttcagaagattcctggaggtttatgagggagaaagaaaagaacccgataataacctatggcatcctgctccggcttatgcctttgagaaagatggaaacatcaaagttaagataacaaaaaatgaagaaacatcgaaatcaaaagatgatacttcaagtgatgatattgcctacgcagagctgagcgtttatgaattaagaaatctcgtttattgttgcctgaatggcaaaaaagatgcagcaaataatatcatcagggattatgttttcaactataaagcttttttaaaagatttagaaaacaaggatttttcagaaattgatgattatacagcacaattggaagaacgaaaacaacaactccaaaacaaattatctgaatataacctacaattgcatcagcttcccaaaaaaatcagaaaaattttactggatgaaaaaatccaggactataagtctcacaccattcaaaaaataaaggacaggcaggaagaaaacaaacgtattctgggaaaaatcaaagctcagaaacaaatgagcaaagaaaacgacaaagatagtcaacaaaaaaatactctaaaaaccggccaattggcaagcgaattagccaatgatattcaaaactatctgcctgagaattacaaactggaactatttcaatacagggatttgcaaaaacaattggcttattacaggagaaaggaaatatatatattactcaatcaaaattatgcattgacttaccatgaacagcaagacaggaatgaaaattttaatgatttgtattataaaaagaaacatcctttcttacaccacgtgttgacacgaaaagataacgatgatatcttttcttttgcattcaactattttaaatctaaagagatatggctggaaaaagtccgtaaaaaagtaattgggcttaatgacactgatattccaaaatattccgaacttttttattattttaaaccgggcacctcagtaaatgaaaagggagaaaaaatttactaccgcaaatacgatgaccactatttaaataaactcattcaaagacacttaaaacaagatcacgttatcaatattccccggggcatattaaatcagttcatctgcccggagaaagaatcatatgaacaaaaaaacaatcctattcaaaaaatcgcagatcaatatccttccacacaggatttttataaatttcctcgtttttatcatccaacaggtgaagtattaaccgtggaagatattaactataaactggtagaattaagtaaagataaagatcatccacacaacaatgacaaaaaagagcataaaaaagcatacaaccagcttaaaaaatatcttaaaaaagaaaagactatacgatatattcagtcctgtgaccgtgttttattggaaatgattaaatattatctgaataattattttaaaaagtctaatgaggagtttgaacttgatttaacagatattgagttacgggatttatttaaatatgatgaaaccaatgaatccatccataacaaactggatcagaaaatgattacattgaaattccatttgaatgggcaatcttttcttgcagaagacaaactcaacaattttgggaaactccatcgttatatttatgacgaaagatttataagtatttttaaatacaaagggaacaaagcatttgaaggagtcaaaacagaaagcatctatagtcaattggaaaaaattttagaagcttttgccaaagaacaactggaattatttgaatatgtgcagcaatttgaaaaaacgataacaactaattttgaaaataaagtaaatcaaaaaagaacagaagaaaatgcaaggcgggaaaaaaatgggaaaccgttaatctcagaacattactttccgatttcaatattactttcactgacagaggaatggggctttatttccggaaaaaaccgaaatttcatcaatacagcccgcaacagtgctgcacataataaactggatgataaatacattgaaatgcttaaagatagagaatatgaaaatgattattttggggcagcctcaaaaatttttaatgaccttacggaaaaaatcagaactgcatag

SEQ ID NO: 46 - DNA, 3162 bp, Artificial Sequence
  Description of Artificial Sequence: Synthetic polynucleotide
atgaccctgccggacaaacagcaaagcaccatctacagcatggaccgtagcgaggataagtacttctttgcgctgtatctgaacattgcgcagaacaacgtggacaaagttctgaaggagttcgatagctggtttaacagcctgaacgaaaccagccagggtaaatacaacagcgcgcaggcgaagtggctggacaaccgtctgccgggcagcgacagcgatgtgctggaggcgaaagaacgtctggtttatctgcgtcgtttctttccgttcatcgaaaccgaatttaccaccaaagaataccacggttatcgtgagaagctgctgatgctgttcgaacgtctgaacgattttcgtaacttctttacccacgtgcactacgaacgtaacgagctggaatttagccgtaacaagaaaatgttcgagtttctgaacgaggttaaggaaatcgcgctgaacaaactgaaccagcacccgtactatctggacgataacattctgaaccacctgcacgacccggatcagcgtttcaactttcaaaaggagaacaacatcaaagacgcgattaacttctttgtgtgcctgttcctggaaaacaagcacgcgcacgagtacctgaagaaacagaaaggttataagagcagccacaacccggaacaccgtgcgaccctgaaaacctacaccttttatagcatcaagctgccgcgtccggttttcgagagccgtgacatgaaactgcgtctgattctggatgcgctgaacgaactgaagaaatgcccgaagcaactgtacgatcacctgagcgagaaacaccagaagctgtgccaagtggaaagcgttaaacagaaggagaacgaggaaagcggcgaaaccgaggaaatcaaagagtatatcccgttcattcgtcacgaagacaagtttccgtactatgcgctgcgtttcattgacgatctggagctgctgaaagacatccgtttcaaaattaagcgtggtctgggcaaggagttcttccacacccacgaaaccgcgacccagccggtggttcgtaacaagaaagtgttcacctttcgtcgttttctggaagtttacgagggtgaacgtaaagaaccggacaacaacctgtggcacccggcgccggcgtatgcgttcgagaaagatggcaacatcaaagtgaagattaccaagaacgaggaaaccagcaaaagcaaggacgataccagcagcgacgacatcgcgtacgcggaactgagcgtgtatgagctgcgtaacctggtttactgctgcctgaacggtaagaaagacgcggcgaacaacatcatccgtgattacgttttcaactacaaagcgtttctgaaggacctggaaaacaaggatttcagcgagatcgacgattacaccgcgcaactggaggagcgtaagcagcaactgcagaacaaactgagcgaatataacctgcagctgcaccaactgccgaagaaaatccgtaaaattctgctggacgagaagattcaggattacaaaagccacaccatccaaaaaattaaggaccgtcaggaagagaacaagcgtatcctgggtaaaattaaggcgcagaaacaaatgagcaaggaaaacgacaaagatagccagcaaaagaacaccctgaaaaccggtcaactggcgagcgagctggcgaacgacatccagaactacctgccggaaaactataaactggagctgttccaataccgtgatctgcagaaacaactggcgtactatcgtcgtaaggagatctatattctgctgaaccagaactacgcgctgacctatcacgaacagcaagaccgtaacgagaacttcaacgatctgtactacaagaaaaagcacccgttcctgcaccacgtgctgacccgtaaagacaacgacgacatcttcagctttgcgttcaactacttcaaaagcaaggaaatttggctggagaaagtgcgtaaaaaggttatcggcctgaacgacaccgatattccgaagtacagcgaactgttttactacttcaagccgggcaccagcgtgaacgagaaaggcgaaaagatctactatcgtaagtacgacgatcactatctgaacaaactgattcagcgtcacctgaagcaagaccacgttatcaacattccgcgtggtatcctgaaccaattcatttgcccggagaaggaaagctacgagcagaaaaacaacccgatccagaagattgcggaccaatatccgagcacccaggatttttacaaattcccgcgtttttatcacccgaccggcgaagtgctgaccgttgaggacatcaactacaaactggtggagctgagcaaagacaaggatcacccgcacaacaacgataaaaaggagcacaaaaaggcgtacaaccaactgaaaaagtacctgaaaaaggaaaagaccatccgttacattcagagctgcgaccgtgttctgctggagatgatcaagtactacctgaacaactacttcaaaaagagcaacgaggagttcgaactggacctgaccgatattgagctgcgtgacctgtttaaatacgatgaaaccaacgaaagcatccacaacaagctggatcaaaaaatgattaccctgaagtttcacctgaacggtcagagcttcctggcggaagacaaactgaacaacttcggcaagctgcaccgttacatctatgatgagcgtttcatcagcatcttcaagtacaagggtaacaaagcgtttgaaggcgttaagaccgagagcatctatagccaactggaaaaaattctggaggcgttcgcgaaggagcagctggaactgttcgagtacgtgcagcaatttgaaaaaaccatcaccaccaactttgagaacaaggttaaccagaaacgtaccgaggaaaacgcgcgtcgtgagaagaacggcaagccgctgattagcgagcactacttcccgatcagcattctgctgagcctgaccgaggaatggggttttatcagcggcaaaaaccgtaacttcattaacaccgcgcgtaacagcgcggcgcacaacaagctggacgataaatacatcgaaatgctgaaggaccgtgagtacgaaaacgattattttggcgcggcgagcaaaatcttcaacgacctgaccgagaagattcgtaccgcgtag

SEQ ID NO: 47 - DNA, 3492 bp, Artificial Sequence
  Description of Artificial Sequence: Synthetic polynucleotide
atgactacaatagaaaactttagaaaatacaacgccgataaatcgtttaaaaatattttcgatttcaaaggtgagattgctcctatagcagaaaaatcgtcgagaaaccttgaactaaagctcaaaaacaaagtaggcgtagaaacatcggtacattattttgccatagggcatgctttcaaacaaatagacaaagaagcggtatttgattatatttatgatgaagaaaccgactcaaaaaaacctcatcggtttacttcgctcaaacagtttgatgagcaattttgcaaagaattaaaaaatatagtttcaaccattagaaatattaactcccattatattcacgactttgggcaaataaaatgcgatacactttctctacaattaattacatttcttaaagaaagtttcgagttagcggttattcagacgtatttgaaatcaaaagaaagtacaaaagatgctatgactacccaagatttttttgatgctcccgataaggataaaaaaatagttgaatttcttaaagaaaggttttatgctattgattctgaaaagaaaaacttagaaagctatcaaaaccatattaatcgttcaaaatattttggcacacttacaaaagaacaggctattgaaaccattctctttggcgaggtggtagatcctaattttaaatggaagttgaacgagacacatatagcttttcctatttctgtcggaaaatatctttcctatcatgcctgtttattcatgctcagtatgtttctgtacaagcacgaggcggagcaattgatttctaaaataaaagggttcaagaagtcgaaaaatgatgaagataaactcaaacgcaatattttcacctttttctcaaagaaattcagtagcgaagatattaaaagcgaacaagctcatttggtaaagtttcgagatattgttcaatacctcaaccattacccattggattggaataaatatatagaattggaatcagcttacccctcaatgactgataaactgaaagctaagattattgaaatggaaattgatcgttcttatccaaattttgtaggaaatacaagatttcatacttatataaaatttgagttatggggaaaaaaattctttggaaataaaatttttaaagaatattgcgattgttcttttaccccaaaggaattagaagaattcaaatatgaaaaagatacttgcggaaaagtaaaagatgcggaattaaaattaaaagaaaaacatctattaaaacatgatgaaataaaaaaacttgaagataaaatagaggaaaacaaagacaagcccaacaatattactttaaccctcgatacccgaattaaaaaaaacctcttgttcacatcttacgggcgaaatcaagaccgatttatgcaatttgccactcgctatttagcagaaacgaactactttggcaaggatgcacaattcaagatgtaccgattcttttcatcggtagataataccaatgaaattgaatctcaaaaagagaagctagataaaaaactgattaataaaaaacaatttgacaacctcagatttcacgacggcagactcacttacttcgcaacatttaaagaacatctggtgcgttacgaaaactgggatacgccgtttgtagaggaaaacaatgcggtacaggttcaaatcacatttaattatgaagaaatacttaaagatacaaatcaaacaattttagtttacataacgaaagtaatatctattcagagaagcttaatggtttactttcttgaagatgcactaaaatcaaacacattggcaaattcggaaggagtaggggtaaaattgttgtttaattattatatgcatcacaaaaaggaatttgcggagaataaacatgaacttgaaaacaacgataaagaaagtattgataatacttacaagaaaatattcccaaaacgattgattaataagtttgttgcagttagcccaaatgacccaaaacagcaatctgtttatgaaagtatactagaaaaggcaaagaaatcggaagagagatataaagacctacgtgcgaaagcagaaaaagacaaacgattagaagatttcgataaaagaaacaaagggaaacagttcaagttacagttcgttcgcaaggcatggcacctcatgtacttcagagatatatacaatttatatgctattgacgggaaacccgaaaatcaccataaacatttacacataactcgcgaagaatttaataatttttgccgttatatgtttgctttcgatgaagtgccgcaatacaaactactgcttaaaaacatgctcgcagaaaaacattttttggacaacaaggcgtttgaaaccctgttcgatagcagccatgatttgaattctatgtattgcaaaaccaaagaaaagtttaaagtttggatgagccaacccaaggaaaccagcaatgataaagaacattatacccttgccaattatgaaaagtttttcaaagacaaaatgttttacataaatctctcgcatttcagagatttcctcaaagagaaaaaaaggtttataatagcaaatgataagattgttttcaaatcgcttgaaaacaaccagtatctgatgcaagactactatatagaagaaacaccagcaaaagaaaagtataagacaaaagaagaatacaaggcaaacaagaatttgtataacgaactacgcaaaagcagacttgaagatgcattgctctatgagatggcaatgcactacctcggcatggagaaagatattacaaaaaatgcaaaagttcctgttcaaaaaattctatctcaagatgtatcatttgaaattaaagacttaaaaaacattaccaactacaccttatccgtcccttttaagaaattggaatcctatttaggtttgatggcatttaaggaaaaacaagaacaggaatataaaggaagctatatgattaatcttgttgaatatttaaagaaaattgaacaagataaagacacaaaaaaagaaataaaacaaatatggaatgacataaatggaaataaaaagctttcgctcgaccaactcaataaatttgatgctcatataatatcaaactccattaaatttaccagagttgctattctttttgaacaatattttatcgttaagcataatcatagcataataaaagacaacagaatttcttttgaagaaattgaagaaattaaggaatattttgtaaaactcacccgaaacaaagcatttcattttaacattccagaaaagccttattcgtcattattaaaagaaattgaaaagagatttattcaaaaagaagtaaagattcagaatcctaaaagtttcgatgaaataaagcttaatgaaaagtatatctgctcagcatttcttaattctttatatgatgtatatttcaattttaaagaaaaagatgaaaagaaaaaacggtacgatgcagaacagaaatattttactgcgataattgcataa

SEQ ID NO: 48 - DNA, 3492 bp, Artificial Sequence
  Description of Artificial Sequence: Synthetic polynucleotide
atgaccaccatcgagaacttccgtaagtataacgcggacaagagcttcaagaacatcttcgatttcaagggcgagatcgcgccgattgcggaaaagagcagccgtaacctggagctgaaactgaagaacaaagtgggtgttgaaaccagcgtgcactacttcgcgatcggccacgcgtttaagcagattgataaagaagcggttttcgactacatctatgatgaggaaaccgacagcaagaaaccgcaccgttttaccagcctgaagcagttcgacgagcaattctgcaaggaactgaaaaacatcgtgagcaccatccgtaacattaacagccactatatccacgatttcggccagattaaatgcgacaccctgagcctgcaactgattaccttcctgaaggagagctttgaactggcggtgatccagacctacctgaagagcaaagagagcaccaaagatgcgatgaccacccaagacttctttgatgcgccggacaaagataagaaaattgttgagttcctgaaggaacgtttttacgcgatcgacagcgagaagaaaaacctggaaagctaccagaaccacatcaaccgtagcaaatatttcggtaccctgaccaaggagcaagcgatcgaaaccattctgtttggcgaggtggttgacccgaacttcaagtggaaactgaacgaaacccacatcgcgttcccgattagcgttggtaaatacctgagctatcacgcgtgcctgttcatgctgagcatgtttctgtacaagcacgaggcggaacagctgatcagcaagattaaaggcttcaagaaaagcaaaaacgacgaggataagctgaaacgtaacatcttcaccttctttagcaagaaattcagcagcgaggacatcaaaagcgaacaggcgcacctggtgaagttccgtgacattgttcaatacctgaaccactatccgctggattggaacaaatacatcgagctggaaagcgcgtatccgagcatgaccgacaagctgaaagcgaagatcattgagatggaaattgatcgtagctacccgaacttcgtgggtaacacccgttttcacacctatatcaagttcgagctgtggggtaagaaattctttggcaacaagatcttcaaagaatattgcgactgcagcttcaccccgaaagagctggaggaatttaagtacgaaaaagatacctgcggcaaagttaaggacgcggagctgaaactgaaggaaaaacacctgctgaaacacgatgagatcaagaaactggaagacaagattgaggaaaacaaggataaaccgaacaacattaccctgaccctggatacccgtatcaagaaaaacctgctgttcaccagctatggtcgtaaccaggaccgtttcatgcaatttgcgacccgttacctggcggagaccaactattttggcaaggacgcgcagttcaaaatgtaccgtttctttagcagcgtggataacaccaacgagattgaaagccagaaggaaaaactggacaagaaactgatcaacaagaaacaattcgataacctgcgttttcacgacggtcgtctgacctacttcgcgacctttaaggagcacctggtgcgttatgaaaactgggataccccgttcgttgaggaaaacaacgcggtgcaggttcaaatcacctttaactacgaggaaattctgaaagacaccaaccagaccatcctggtgtatattaccaaggttatcagcattcaacgtagcctgatggtttacttcctggaggatgcgctgaaaagcaacaccctggcgaacagcgaaggtgtgggcgttaagctgctgttcaactactatatgcaccacaagaaagagtttgcggaaaacaaacacgagctggaaaacaacgataaggagagcatcgacaacacctacaagaaaatcttcccgaagcgtctgattaacaaatttgtggcggttagcccgaacgacccgaaacagcaaagcgtgtatgagagcatcctggaaaaggcgaagaaaagcgaggaacgttacaaggacctgcgtgcgaaagcggagaaggataaacgtctggaagacttcgataaacgtaacaagggtaaacagttcaaactgcaatttgttcgtaaggcgtggcacctgatgtactttcgtgacatctacaacctgtatgcgattgatggcaaaccggagaaccaccacaagcacctgcacatcacccgtgaggaattcaacaacttttgccgttacatgttcgcgtttgatgaagtgccgcagtataagctgctgctgaaaaacatgctggcggagaaacacttcctggacaacaaggcgttcgaaaccctgtttgatagcagccacgacctgaacagcatgtattgcaagaccaaagagaagtttaaagtttggatgagccaaccgaaagagaccagcaacgacaaggaacactacaccctggcgaactacgaaaagttctttaaggacaagatgttctacatcaacctgagccacttccgtgattttctgaaagagaagaaacgtttcatcattgcgaacgataagatcgtgtttaaaagcctggaaaacaaccagtatctgatgcaagactactatattgaggaaaccccggcgaaggagaaatacaagaccaaagaggaatataaggcgaacaaaaacctgtacaacgaactgcgtaagagccgtctggaggatgcgctgctgtacgaaatggcgatgcactatctgggtatggagaaagacattaccaagaacgcgaaagtgccggttcagaagatcctgagccaagacgtgagcttcgaaatcaaggatctgaaaaacattaccaactacaccctgagcgttccgttcaagaaactggagagctatctgggtctgatggcgtttaaggaaaaacaggagcaagaatacaaaggcagctatatgattaacctggtggagtacctgaagaaaatcgaacaggacaaagataccaagaaagagatcaagcaaatttggaacgatatcaacggcaacaagaaactgagcctggatcagctgaacaaattcgacgcgcacatcattagcaacagcatcaagtttacccgtgtggcgatcctgttcgaacaatacttcatcgttaagcacaaccacagcatcattaaggacaaccgtatcagcttcgaggaaatcgaggaaatcaaggagtacttcgttaagctgacccgtaacaaggcgttccactttaacatcccggaaaagccgtacagcagcctgctgaaggagatcgaaaaacgtttcatccagaaagaggtgaagatccaaaacccgaaaagctttgatgagattaagctgaacgaaaaatacatctgcagcgcgttcctgaacagcctgtacgacgtttacttcaacttcaaggagaaggacgaaaagaaaaagcgttacgatgcggaacagaagtattttaccgcgatcattgcgtag

SEQ ID NO: 49 - DNA, 3375 bp, Artificial Sequence
  Description of Artificial Sequence: Synthetic polynucleotide
atggaaactacacaaacatctgaaaacaagagaaggtcacttgcaactgaccctcagtattttggcggctatttgaatatggcacggctaaatatttataacattaataattatctggcggaggagtttggactttcccaactcccggaagatggatatattaaaaacagttttttatgtaaccaaaaacaaacaaaacttaactggaaccgggttttttcaaaggcagtaacttttttacccatcctgaaggtttttgattctgagtcactaccgaaatcggaaaaagaagataaatcaacacccgaaaccggcaaggatttcgcaaaaatggcagattccctgaaagttctcttttccgaaattcaggagttcagaaatgattattctcattactactctaccgaaaaaggcactgataggaaaattaccatttcaaatgaactggctgattttctcaagtttaattacaaaagagccattgaatatacaagggtgagatttaaagatgtgtacaccgacgatgattttaatgtggctgctaataaaaaaatggtaatcggcggggttattaccaccgaaggactggtttttctaacttccatgtttcttgaacgtgaatacgcatttcagtttatcggtaaaattacaggattgaagggtacacaatatgtgggtttcagggcatttcgagatgttttaatggctttttgcatcaaacttccacacgaaaaactaaaaagcgacgactttatccagtcgtttacgctcgacataattaatgaattaaaccgttgtccaaaaacgctttacaatgtaattaccgaagaagaaaaaaggaaattcagaccgcagattgaacctgaaaagattgacaatttactgaaaaacagcgggattgaactggaagagtatgacgaaaatttcgatgattatgtggaatcgttgaccaggaaaatacgtcacgaaaacaggttcaactattttgcattacgttatattgacgaaaataaaatttttgggaaataccgttttcaaatcgatttaggaaaactggtgattgatgaatatcctaaaaagttcttcaacgaagaagttcagcggcggataatcgaaaatgcaaaagcttttgacaaactgagtgatttggttgatgaaacagcgattttaaagaagattgatatacaaaaccaccaggtttattttgaaccttttgcaccacattacaataccgaaaacaataaaattgccttattatcaaaaagtgatattgcaagagtgcgaaaggtaaaaaccaaaacaggtgtagaaagaaaaaacctgtttcagcctttgcctgaagcttttttgagctgtgccgaattgtataaaatagtgttgctggaatatttaaaacctggtgaagctgaaaaactggttacagattttattcttgccaacaacagtaaactgatgaatatgcagtttattgaactggtgaaaaaacaaatgcccggttggattgtatttcaaaaagaaaccgatacaaaaagcagactggcttattcacaaattaactttaatgaacttttaagcagaaaaagccaattgaataaagtattagccgaacacaatttaaacgataaacaaattccttcaaaaatattggaattctggctgaacatcagtgatgtaaaacaacagtttactaccggggaacggataaaactgataaagcgggattgtatgaagcggttgaaagcgcttaaaaaattcaaaaccaccggaaagggaaaaatcccgaaaattggcgaaatggccacattcctggcaaaagacattgttgacatggttattggaaaagaaaagaaacagaaaataacttcgttttactacgacaaaatgcaggaatgtctggccttgtatgccgaccctgaaaaaaagaaaacatttattcatattatcacccatgaacttggattgtatgaaaaagacggccacccgtttttaaaccgcataaatttcaacgaattgcgttacacccgcgatatttatgaaaaatacctcgaagaaaagggagaaaaaatggtgaaattttataatgccaggcgaggaaattatacggagaaagataaatcgtggttaagggaaactttttacactttggtggaaaaagaaattaaagggaaaaagaggataatgaccgaagtggttttaccttccgacaaatcaaaaatcccattcacgttacttcaattagaagaaaaaacaacgtattctttggccgactggctgcaaaacattaccaaaggaaaagagcacggtgatggaaaaaaaccggtaaaccttccaaccaatctttttgacgaaacaattaccagtttgctgaagacagaacttgataataaacaggcgctttaccccgaaaatgccaaaatgaacgaattgtttaaactttggtggatgggccgtggcgacggggtgcaacatttttatgacgccgaaagggaatattttgtttttgaacaacctgtaaaatttaaacccggctcaaaggcaaaattctctgattattactgcattgcgcttacaaaagcatttaaggaaaaggagaaaacagctacaaaagagagaaaacaggctcctgaacttgatgaagttgaaaaaacctttcagcaggcaattgccggaactgagaaagaaataagggaattacaggaagaagacagggtttgtgcgcttatgcttgaaaaactcatcagcagggaaaagcatattaccgttaaattggaatcgattgagaatttgttaaaggaatcagtagttgtaaaacaaaccgttaatggtaaactgtatttcgatgaaaacgggaacgagataaaagacaaatcgaacccagtaataaccaaaaccattgttgacaaacggaaaggaaaagattacggtttactccgtaaatttgcaaacgaccgccgtgtgcccgaactgtttgaatatttttccggcgaagaaataccgctggaacagttaaaaaaagaacttgatgggtacaacattgccaaacacctggtttttgatgttgttttcagacttgaggaaaaactgattaaaagtaaccggaatgaaattatttcctattttacagatgataaaggaaatgcaaaaggcggaaacatacagcacctgccttatttaaacctgctgaaagaaaaggatttggtaacgcccggtgaaatggcttttttgaacatggtacgcaactgtttttcgcacaaccagttcccgaaaaagagtattatgaaaaaagttgttaagcccggtgaaaacaattttgcaaagaaaattgctgatatttacaatgaaaaaattgaggctttgatattaaaacttgcataa

SEQ ID NO: 50 - DNA, 3375 bp, Artificial Sequence
  Description of Artificial Sequence: Synthetic polynucleotide
atggaaaccacccaaaccagcgagaacaaacgtcgtagcctggcgaccgatccgcagtacttcggtggctatctgaacatggcgcgtctgaacatctacaacattaacaactatctggcggaggaattcggcctgagccaactgccggaggacggttacatcaagaacagctttctgtgcaaccagaagcaaaccaaactgaactggaaccgtgttttcagcaaagcggtgacctttctgccgattctgaaggttttcgatagcgaaagcctgccgaagagcgaaaaagaggacaagagcaccccggagaccggcaaggattttgcgaaaatggcggacagcctgaaagtgctgttcagcgaaatccaggagtttcgtaacgattatagccactactatagcaccgaaaagggcaccgatcgtaaaatcaccattagcaacgagctggcggacttcctgaagtttaactacaaacgtgcgatcgagtatacccgtgttcgtttcaaggacgtgtacaccgacgatgactttaacgttgcggcgaacaagaaaatggttatcggtggcgtgattaccaccgaaggtctggtgttcctgaccagcatgtttctggagcgtgagtacgcgttccaatttatcggcaagattaccggcctgaaaggtacccagtatgttggtttccgtgcgtttcgtgatgtgctgatggcgttctgcatcaaactgccgcacgagaaactgaagagcgatgacttcattcaaagctttaccctggacatcattaacgaactgaaccgttgcccgaagaccctgtacaacgttatcaccgaggaagagaaacgtaaattccgtccgcagatcgaaccggagaagattgataacctgctgaaaaacagcggtatcgaactggaagagtacgacgagaactttgatgactatgtggaaagcctgacccgtaaaattcgtcacgagaaccgtttcaactactttgcgctgcgttatatcgatgagaacaagattttcggcaaataccgttttcaaatcgatctgggcaagctggttatcgacgaatacccgaagaaattctttaacgaagaggtgcagcgtcgtatcattgaaaacgcgaaggcgttcgataaactgagcgatctggttgacgagaccgcgatcctgaagaaaatcgacattcagaaccaccaagtgtacttcgaaccgtttgcgccgcactataacaccgagaacaacaagatcgcgctgctgagcaaaagcgacattgcgcgtgttcgtaaagtgaagaccaaaaccggcgttgagcgtaaaaacctgttccagccgctgccggaagcgtttctgagctgcgcggagctgtacaagatcgttctgctggaatatctgaagccgggtgaagcggagaaactggtgaccgatttcattctggcgaacaacagcaaactgatgaacatgcagtttatcgagctggttaagaaacaaatgccgggctggattgtgttccagaaggaaaccgacaccaaaagccgtctggcgtatagccaaatcaactttaacgaactgctgagccgtaagagccagctgaacaaagttctggcggagcacaacctgaacgataagcagatcccgagcaaaattctggaattctggctgaacatcagcgacgtgaagcagcaatttaccaccggcgagcgtatcaaactgattaagcgtgactgcatgaaacgtctgaaggcgctgaagaaattcaaaaccaccggcaagggcaaaatcccgaagattggcgagatggcgacctttctggcgaaagatatcgttgacatggtgatcggcaaggaaaagaaacaaaagatcaccagcttctactatgataagatgcaggaatgcctggcgctgtacgcggacccggagaagaaaaagaccttcatccacatcatcacccacgaactgggcctgtacgagaaagatggtcacccgttcctgaaccgtatcaactttaacgagctgcgttatacccgtgacatttacgaaaagtatctggaagagaaaggcgagaagatggttaaattctacaacgcgcgtcgtggtaactataccgaaaaggataaaagctggctgcgtgagaccttttataccctggtggaaaaggagatcaaaggtaaaaagcgtattatgaccgaggtggttctgccgagcgacaagagcaaaatcccgttcaccctgctgcaactggaagagaaaaccacctacagcctggcggattggctgcagaacattaccaagggcaaagaacacggtgacggcaaaaagccggttaacctgccgaccaacctgttcgatgaaaccatcaccagcctgctgaagaccgagctggacaacaaacaggcgctgtacccggaaaacgcgaagatgaacgagctgttcaaactgtggtggatgggtcgtggcgatggtgtgcaacacttttacgacgcggagcgtgagtatttcgtttttgagcagccggtgaagttcaaaccgggtagcaaggcgaaatttagcgactactattgcatcgcgctgaccaaagcgttcaaggaaaaagagaagaccgcgaccaaggaacgtaaacaagcgccggagctggatgaagttgagaaaacctttcagcaagcgatcgcgggcaccgaaaaggagattcgtgagctgcaggaagaggaccgtgtttgcgcgctgatgctggaaaagctgatcagccgtgagaagcacattaccgtgaaactggaaagcatcgagaacctgctgaaggaaagcgtggttgtgaaacaaaccgtgaacggcaagctgtacttcgatgaaaacggtaacgagattaaagacaagagcaacccggttatcaccaaaaccattgtggataagcgtaagggcaaagactacggtctgctgcgtaagtttgcgaacgaccgtcgtgttccggaactgttcgagtattttagcggcgaagagatcccgctggaacagctgaaaaaggagctggatggttacaacattgcgaaacacctggtgttcgacgttgtgtttcgtctggaagagaagctgatcaaaagcaaccgtaacgagatcattagctatttcaccgatgacaagggcaacgcgaaaggtggcaacattcaacacctgccgtacctgaacctgctgaaggaaaaagatctggttaccccgggcgagatggcgttcctgaacatggtgcgtaactgcttcagccacaaccagtttccgaaaaagagcatcatgaaaaaggttgtgaagccgggtgaaaacaactttgcgaaaaagatcgcggacatttacaacgaaaaaatcgaggcgctgattctgaagctggcgtag

SEQ ID NO: 51 - DNA, 3276 bp, Artificial Sequence
  Description of Artificial Sequence: Synthetic polynucleotide
atgtcagattctcaactgaaaccacgttacaccctcggtctggacctcggcgtttcatcgatcggctgggccatgatcgagccggttgacacagcgggaccggccaaaatcgtccgcagcggggtccatctgtttgatgcgggcgtcgagggcagcgaagacgatatcgagcaaggccgcgagaaagcgcgtgccgctccacgccgcgacgcccgccagcagcgtcggcagacctggcggcgggccgcacggaaacgaaagctgctgcgtcttctgatccgcgctcgcctgctgccggattcggaaaccggcctgcaaacgccggaggaaatcgatcattacctcaaatccgttgacgccgacctacgcgtcacctgggaacaggacattgatcatcgcgcccaccagttgctgccctaccgcctgcgcgccgaagcgatccggcgaaggctcgagccgtacgagatcggccgcgccttgtaccacctcgcccagcggcgcggatttctgagcaaccgcaagactgacgacgacggcggcgatggcgacgacgacacgggcgccgtcaagcaaggcatcgccgagttggaaaagcggatggaccaagccggcgcggagacgctcggcgaatacttcgcctcgcttgatcccaccgacggcgcgtcccggcgcatccggggccgctggaccgcgcgtccgatgtacgagcatgagttcgaccgcatctggtcggagcaggccggccaccactcgggccgcatgaccgacgaggcgcgtcagcagatccgccacgccatcttttttcagcgaccactcaaaagtcagcgtcacctgatcggccgttgctctttgatttctaaaaaacggcgcgcccccatggcccatcgtctgttccagcgattccgcctgcggcaaaaggtcaacgacctgcagatcatcccgtgcaggcgcgtcgaggtcgacgccgttgacaagaagaccggcgaagtcaaaatcgaccccaaaaccgaccagcccaaacgcgtcaagcgctgggtccccgatcccacccagccgcctcgcccgttgaccgacgacgagcgggccgcggcgctcgagcgcctcgaacatggcgacgcgacttttcatcagctccgtcaggcgggagccgcgccaaaggcctcacgctttaacttcgagaccgagggcgagtcacggcttccgggtctgcgaaccgatgaaaagctgagagaaatattcggcgaccgctgggacgcgatggatgagcgagtaaaagacgccgtcgtcgaggactgtctttcgatcgtccggggcgacacgatggagaggcgaggccgcgaggcgtgggggttgtcggccgacgaggcccgcgccttcgcccgtgtcaagctggaggaaggctacgcccggctgtcccgcgcggcgatgcggcggctgatgcctcacctgcggaacggcgtcccgttcgcatcggcacgcaaacaggaatttcccggatccttcgcgaccaaccccaccgtcgacaccctcccgccactggacaaggcgttcaatgagccggtcagtcccgcggtcgcgcgggcgctgtcggagctgcgcggcgtggtgaatgcgatcatccgccgccacggcaagcccgcccatatccggatcgagctcgcccgcgacctgaagcgtggccgcaaacgccgcgacgccatcagtcgacagatcgccgcccggcgaaagcagcgggaggccgcggccgaacggctcatcgagcgttacccccacctcggcgcgtcggcccgcgacgtctcccatatcgacgtgctcaaagtcgtcctcgccgacgagtgccgctggatctgtccgtttaccggacgggcgttcggctggaccgatgtcttcggccccagcccgacgatcgacatcgagcacatctggccattcagccgatcgctcgacaattcctatctcaacaaaacgctctgcgacgtgaacgagaaccgcaaaatcaagcgaaaccagatgcccaccgaagcctacggccccgaccggctcgaccagatcctccagcgcgtctcccgcttcaccggcgacgccgcacagatcaagctggaacgcttccgcgccgagtcgatccccgccgatttcaccaatcggcatctcaccgagtcccgctacatctcgaccaaggccgccgaatatctcgccctgctttacggcgggcttgcagacgacgagcgcaatcgccgcattcacgtgaccacgggcgggttgaccggctggctgcgtcgggaatgggggatgaacgccatcctctccgacgatgatgagaaagaccgaagcgaccatcgccaccacgccgtggacgccctggtggtcgccttcacgtcccagggcgcggtccagcggttgcagaaggcggccgagcgggccgacgaccggggcatgcgccggcttttctccggcatcgaagcgccgtttgatctcgccgacgcacgtcgcgcgatcgagagcatcgtcgtcagccaccgaaaacgaaacaaggcccgcggcaagttccatagagatacgatctacagccagcccctgcccggcaaggacggcaggaagggccaccgcgtccgcaaggaactgcacaaactcaaggaaaaccagatcaaggacatcgtcgacccccgcatccgcgacgtggtcggccaggcgtatcagaagctgaaaaccgccggcgcgaggaccccggcccaggccttcagtgacccggacaaccgccccgtcctgccccacggcgaccgcatccgccgcgtccgcatcttcgtcagcgccaagccggacgtgatccccggcaaagacgcgcccaaatcacgccgtcgctgcgtcgatctacagtccaatcaccacacggtgatcatggccaaactgaacgcccgcggcgaggaaaagacatgggtcgatgaaccggtcgccttgctggaggcgatggaccgggtccgcgacggcaagcctctggtctgtcgcgacgtgccgaagggatacaggtttatgttttcgctggcggcaaatgactacgtggaaatggatcgtaaagatggtgatggccgcgatgtctaccgaatccgaggcatctcgaaaggagacattgaagtcgtgcagcaccatgacggcaggacacaaacgatccgcaaggccgccaaggaactggatcgagtccgcggatcgacacttcagaaacgtcacgcccgaaaggtgcacgtgaactatctcggggaggtgcacgatgccggcggctga

SEQ ID NO: 52 - DNA, 3276 bp, Artificial Sequence
  Description of Artificial Sequence: Synthetic polynucleotide
atgagcgacagccaactgaagccgcgttacaccctgggcctggatctgggtgtgagcagcatcggctgggcgatgattgaaccggttgacaccgcgggtccggcgaagattgttcgtagcggcgttcacctgttcgatgcgggtgtggaaggcagcgaggacgatattgaacagggtcgtgagaaggcgcgtgcggcgccgcgtcgtgatgcgcgtcagcagcgtcgtcagacctggcgtcgtgcggcgcgtaagcgtaaactgctgcgtctgctgatccgtgcgcgtctgctgccggacagcgaaaccggtctgcaaaccccggaggaaattgatcactacctgaagagcgtggatgcggacctgcgtgttacctgggagcaagatatcgaccaccgtgcgcaccagctgctgccgtatcgtctgcgtgcggaagcgatccgtcgtcgtctggaaccgtacgagattggtcgtgcgctgtatcacctggcgcagcgtcgtggctttctgagcaaccgtaaaaccgacgatgacggtggcgacggcgatgacgataccggtgcggtgaagcaaggcatcgcggagctggaaaaacgtatggatcaggcgggtgcggaaaccctgggcgagtactttgcgagcctggatccgaccgatggtgcgagccgtcgtattcgtggccgttggaccgcgcgtccgatgtatgagcacgaatttgaccgtatttggagcgagcaggcgggtcaccacagcggtcgtatgaccgatgaagcgcgtcagcaaatccgtcacgcgattttctttcagcgtccgctgaagagccaacgtcacctgatcggccgttgcagcctgattagcaagaaacgtcgtgcgccgatggcgcaccgtctgttccagcgttttcgtctgcgtcaaaaagttaacgacctgcagatcattccgtgccgtcgtgttgaggtggatgcggtggacaagaaaaccggtgaagttaagatcgacccgaaaaccgatcaaccgaagcgtgtgaaacgttgggttccggacccgacccagccgccgcgtccgctgaccgatgatgagcgtgctgcggcgctggaacgtctggagcacggtgatgcgacctttcatcagctgcgtcaagcgggtgcggcgccgaaggcgagccgtttcaactttgagaccgaaggtgaaagccgtctgccgggtctgcgtaccgacgaaaagctgcgtgagatctttggcgatcgttgggacgcgatggatgagcgtgtgaaagacgcggtggttgaagattgcctgagcattgttcgtggtgacaccatggagcgtcgtggtcgtgaggcgtggggcctgagcgcggatgaggcgcgtgcgttcgcgcgtgttaaactggaggaaggttatgcgcgtctgagccgtgcggcgatgcgtcgtctgatgccgcacctgcgtaacggtgtgccgtttgcgagcgcgcgtaagcaggaattcccgggcagctttgcgaccaacccgaccgttgacaccctgccgccgctggataaagcgtttaacgagccggttagcccggcggttgcgcgtgcgctgagcgaactgcgtggtgtggttaacgcgatcattcgtcgtcacggcaagccggcgcacatccgtattgagctggcgcgtgacctgaagcgtggccgtaaacgtcgtgatgcgatcagccgtcaaattgcggcgcgtcgtaagcagcgtgaagctgcggcggagcgtctgatcgaacgttatccgcacctgggtgcgagcgcgcgtgatgtgagccacatcgatgttctgaaagtggttctggcggacgagtgccgttggatttgcccgttcaccggccgtgcgtttggttggaccgacgtgttcggtccgagcccgaccatcgatattgaacacatttggccgtttagccgtagcctggacaacagctacctgaacaaaaccctgtgcgatgtgaacgagaaccgtaagatcaaacgtaaccaaatgccgaccgaagcgtatggtccggaccgtctggatcagattctgcaacgtgttagccgtttcaccggtgatgcggcgcagatcaagctggagcgtttccgtgcggaaagcattccggcggattttaccaaccgtcacctgaccgagagccgttacatcagcaccaaagcggcggaatacctggcgctgctgtatggtggcctggcggacgatgagcgtaaccgtcgtatccacgttaccaccggtggcctgaccggttggctgcgtcgtgagtggggcatgaacgcgattctgagcgacgatgacgaaaaggaccgtagcgatcaccgtcatcatgcggtggatgcgctggttgtggcgttcaccagccagggtgcggttcagcgtctgcaaaaagcggcggaacgtgcggatgaccgtggtatgcgtcgtctgttcagcggtattgaagcgccgtttgacctggcggatgcgcgtcgtgcgatcgaaagcattgtggttagccaccgtaagcgtaacaaagcgcgtggcaagtttcaccgtgacaccatttacagccaaccgctgccgggcaaggatggccgtaaaggtcaccgtgtgcgtaaggagctgcacaagctgaaagaaaaccagatcaaagacattgttgatccgcgtatccgtgacgtggttggtcaggcgtatcaaaagctgaaaaccgcgggtgcgcgtaccccggcgcaagcgttcagcgatccggacaaccgtccggtgctgccgcatggtgaccgtatccgtcgtgtgcgtatttttgttagcgcgaaaccggacgttatcccgggcaaggatgcgccgaaaagccgtcgtcgttgcgtggatctgcagagcaaccaccacaccgttattatggcgaagctgaacgcgcgtggtgaggaaaaaacctgggtggatgagccggttgcgctgctggaagcgatggaccgtgtgcgtgatggcaagccgctggtgtgccgtgatgttccgaaaggctaccgtttcatgtttagcctggcggcgaacgactatgtggagatggatcgtaaggatggtgacggccgtgacgtttaccgtatccgtggcattagcaaaggtgacatcgaggtggttcaacaccacgatggtcgtacccagaccattcgcaaagcggcgaaagaactggaccgtgtgcgtggcagcaccctgcagaagcgtcacgcgcgtaaagtgcacgttaactatctgggtgaagttcacgatgcgggtggctag

SEQ ID NO: 53 - DNA, 4698 bp, Artificial Sequence
  Description of Artificial Sequence: Synthetic polynucleotide
atgactaaaattttaggactcgacattggtacaaattcagtgggtggcgcactgattaatttggaagaattcggtaaaaaaggcaatatagaatggcttggtagtagggtaattccagtagatggcgatatgcttcaaaaatttgaaagtggggcccaggtggaaaccaaagcttcctcaagaacacgaataaggatggcaagaagattaaaacatcgttataaacttagaagaacacgcataattcaagtgttcaaattacttaaatgggttgacgaaagtttccccgaaaacttcaaagaaaaaaagaataacgatccaacatttgaatttgatattaatgactatctccccttcactcaagcatcccttgaagaggcaaagaacttattaggaattaccaacaaagatggagaaaccaaagtaccacaggattggattgtttattatttgaggaaaaaagcgctttccgagaaaatctcacttcaggagcttgcccgtatactctatatgatgaatcaaagaagggggtttaaaagtagtagaaaagacttggaggaaacttctattatagattatgaagcatttaaaaaatatacgaataataaccaatatttggatgaaaatggcaatacacttgagacacaatttgttgttactacgaaaattaaatcagtagagcagaagagtgatgagaaagatagtagaggaaattatacatttatcattacagccgaaagtgatagattacaaccttgggaggaaaagagaaagaaaaaacctgattgggaaggaaaggagtttaaacttttaacaactcttaaaacaagaaaaagtggtaaaattgaacaattaaagccaaaggctccttcagaagatgattggaatcttacaatggtggctctggataatgaaattgaagaatccggaaaacaagttggggaattctttttcgataaacttcttaatgacaaaaactacaaaatacgccagcaagtagttaaaagagaaaagtatcaaaaagagctgcgagctatttggaataagcaacttgaacttaatgaagaccttaataaattaaacgaagacccagcattactggaaagaatagcaaaggagctgtatcctacccaaactgaatttaaagggcctaaatataaagaaatcacatctaatgacctttatcatgtatttgccaatgacattatttattatcaaagagacctgaaatcccaaaagagcttgattgatgattgtcgttatgaaaagaaaaagtactttgacaaaaatcttggcaaagaagtaattcagggctataaagttgctccaaaatcaagtcctgaattccaggagtttcgcatttggcaggacataaataatattaaggttattgaaaaagagaaagaaattggtggaaaactctatcctgacattaacgtaactgatgaatatgtaaacaatgaagtaaaagcccgcatcttccagttgttggattcaaaaaaagaagtgtccgaatcccaaattcttaaaacaattgataaaaagctaaaaccgacagcatttaaaattaacttatttgcaaacagggataaactaaagggcaacgaaactaaatcattatttcgtagttatcttgaacagtgtggtcgtgaaaatttgcttaatgaccctgacaaattttacaaattatggcatatactgtactcaatcaatggtaaggatgctgaaaaaggtataagggctgccttaaaaaacccaaaaaatgaatttgatctttccgctgaggtaattgaggaactggcaagtttacccgaattttctaatcagtatgctgcctactcctccaaagccattcataaattattaccattaatgcgttccggtgatcattggaaccatcaaagcatttctcaaaaaatccaggaccgaattaataaaatcatcacaagtgaagaggatgaagaaattgataattacacgagagaccaaattaccaactattttaaaagtcaaaaaaacaaagatatatgggaatgtgaacttgaagattttaaggggcttcctgtctggcttgcttgctacactgtttatgggaaacattcagagaaagataaaaaatcatggaagtcttggaaagaaatagatgttatgaaattagttccaaacaatagtttaagaaatcctattgttgagcaaattgttagagaaacactgcacgtagtaagggatgcttgggaaaaatacggacaaccggatgaaatccacattgaaatgagcagggagttgaaaaatcccaaagatgaacgagaacgtatttcagaaatacaaaataaaaaccgtgaagaaaaagaaaggatcaaaaaactattatttgaattgaaggagggaaatcccaactctcctattgacatcaacaaatttcgtttatggaaaaacaatggaggtaaagaagcacaagaaaaatttgataaccttttcaataacaaagatgaagtttctgtttcaggtgatgagataaagaagtaccggttatgggctgatcaaaatcacacctcaccttataccggcaaacctatcccattaagtaaattatttacgcttgaatatgaaatagaacacatcatcccccaatcaagaatgaaaaatgactcaatgagtaatctggttatatctgaagcggcagtaaacgacttcaaagatagatggcttgcacgaccactgatcgaaaaatatggaggtactcccattgaacataatgggcaaacatttacattgctgaaccaagaagaatttgaaaagcattgcaacaaaactttccaaaatcaacggggtaaacttaagaatctgctcagagaagaagtccctgacgattttgttgaaaggcaaataaatgataacaggtacattaccagaaaattgggcgaattacttgctccggcagccaaagctgatgaaggtattgtttttactacaggttctatcacaaacgaattaaaagataaatgggggttccatacattatggcgtgaattgatgaaacccagatttgaacggttagaacaaattctacaaaaaaaattagttgttccagatgaaaaagacactaataaatttcatttcaatgacccggaacctggcaatcctgtagatattaaacgaattgatcaccggcatcatgcattggatgcattaattgttgccgcaacaacgcgtgctcatattaaataccttaattcacttaattcccataaaaagcgtgaaccttacaagtatttagcaaacaaaggtgtgagggattttatacaaccatggcctgattttacagcggaagtaaaaagtcaattgaaacgccttatcgtatctcataaagtaaattgccaatatgatcccgaacacccggaaaaatccggtgtaatttcaaaacccaaaaatagattcaaaaaatgggtaaaccgggatggcgtttggaaaaaagaataccaatggcaaaaagacaatgaaaattggtgggctataagaaagtctatgttcaaagaacctttgggaatgatatatttaaaagaaatcaaagaagtttcccttaaaaaagcattagaaatacaagctgaaaggcaaaaagggataaaagaccacaccggaagaccaagagattacatttatgataaacttgcaaggcaggaaattcgattcttacttgaagataaatgcggtggagatataaagcaagcagaaaagcaatccagtactttaaaagattccaagagcaatccaattaaaaaagtaagagtcgccttctttaaagaatatgctgcaagtagagttccagttgataattcgtttacatacaaaaaaatcaaggccattccatatgctgaaaaaatcattaatagatgggaagaatgggagcaagatggaaaaaatgagaaaggtcaaaaatttcccaacgatataacaaaatggcccattgaatttttacttaaaaagcacttggatgagtataaaacatcaaatggtaatcctgaccccaatactgcttttacaggagaaggctatgaagcattaactaaaaagaatggagggcaaccgataaaaaaggtaacaacttatgaatcgaagtcagcaccaatcaagtttaatggaaagatcctcgaaactgataaaggtggaaacgtcttttttgtaattgctaaagataaacatacgggtaaacatttggattggtacaccccacctttgtatagcaatgaagcagaagaaggcaaagaaagaggaattataaatcgtttgattaacagagaacccattgctgaagatcaagaggatttggaatatatcacacttgctccagaggatttggtatatgttccggaagaagatgaggatattcggtctattgattggaatggaaaagacaagcagaaagtttttgaaaggacttataaaatggtgagttctacagaaaaagaatgccactttattccccacattgttgcctatccaattttaaaaacagttgaattagggacaaatgataaatcagaaaaagcatgggatggaaaagttgaatatataccaaataaaaaggggaaattaacccgaaaagattccggaacaatgatcaaagaaaattgcgtaaaaataaaattagatagacttggaaacataattaaagtcaatggtaaaccggttaatcattaa

SEQ ID NO: 54 - DNA, 4698 bp, Artificial Sequence
  Description of Artificial Sequence: Synthetic polynucleotide
atgaccaagatcctgggtctggacattggcaccaacagcgtgggtggcgcgctgatcaacctggaggaattcggtaagaaaggcaacatcgagtggctgggtagccgtgtgattccggttgacggcgatatgctgcagaagtttgagagcggtgcgcaagtggaaaccaaagcgagcagccgtacccgtatccgtatggcgcgtcgtctgaagcaccgttacaaactgcgtcgtacccgtatcattcaggtgttcaaactgctgaagtgggttgatgagagctttccggaaaacttcaaggagaagaaaaacaacgacccgacctttgagttcgacatcaacgattatctgccgtttacccaagcgagcctggaggaagcgaaaaacctgctgggtatcaccaacaaggatggcgaaaccaaagtgccgcaggactggattgtttactatctgcgtaagaaagcgctgagcgagaagatcagcctgcaggaactggcgcgtattctgtacatgatgaaccaacgtcgtggtttcaagagcagccgtaaagatctggaggaaaccagcatcattgactacgaggcgtttaagaaatataccaacaacaaccagtacctggacgagaacggtaacaccctggaaacccaattcgtggttaccaccaagatcaaaagcgtggagcagaagagcgacgaaaaagatagccgtggcaactacacctttatcattaccgcggaaagcgatcgtctgcagccgtgggaggaaaaacgtaagaaaaagccggactgggagggtaaagagttcaagctgctgaccaccctgaaaacccgtaagagcggcaaaatcgaacaactgaagccgaaagcgccgagcgaggacgattggaacctgaccatggtggcgctggacaacgaaattgaggaaagcggcaagcaggttggcgagttctttttcgacaaactgctgaacgataagaactataaaatccgtcagcaagtggttaagcgtgagaaataccagaaggaactgcgtgcgatttggaacaagcaactggaactgaacgaggacctgaacaaactgaacgaggatccggcgctgctggagcgtatcgcgaaggaactgtacccgacccagaccgagttcaaaggtccgaagtataaagaaattaccagcaacgacctgtaccacgtttttgcgaacgacatcatttactatcagcgtgatctgaaaagccaaaagagcctgatcgacgattgccgttacgagaagaagaagtatttcgataagaacctgggtaaagaggtgatccaaggctataaggttgcgccgaaaagcagcccggaatttcaggagttccgtatttggcaagacatcaacaacattaaggtgatcgagaaggaaaaagagatcggtggcaaactgtatccggacattaacgttaccgatgagtacgtgaacaacgaagttaaggcgcgtatcttccaactgctggatagcaagaaagaagtgagcgagagccagattctgaaaaccatcgacaagaaactgaagccgaccgcgtttaaaattaacctgttcgcgaaccgtgacaagctgaagggtaacgagaccaaaagcctgttccgtagctacctggagcagtgcggccgtgaaaacctgctgaacgacccggataaattttataagctgtggcacattctgtacagcatcaacggcaaggatgcggagaaaggcatccgtgcggcgctgaaaaacccgaagaacgagttcgacctgagcgcggaagttattgaggaactggcgagcctgccggaatttagcaaccaatacgcggcgtatagcagcaaggcgatccacaaactgctgccgctgatgcgtagcggtgatcactggaaccaccagagcattagccagaagatccaagaccgtattaacaaaatcattaccagcgaggaagacgaggaaatcgataactatacccgtgaccagattaccaactacttcaagagccaaaagaacaaagatatctgggaatgcgagctggaagactttaaaggtctgccggtgtggctggcgtgctacaccgtttatggcaagcacagcgaaaaagataagaaaagctggaaaagctggaaggagatcgacgtgatgaagctggttccgaacaacagcctgcgtaacccgatcgtggagcaaattgttcgtgaaaccctgcacgtggttcgtgatgcgtgggagaaatacggtcagccggacgaaatccacattgagatgagccgtgaactgaaaaacccgaaggatgagcgtgaacgtattagcgaaatccagaacaagaaccgtgaggaaaaagagcgtatcaagaaactgctgttcgaactgaaagagggtaacccgaacagcccgatcgacattaacaagtttcgtctgtggaaaaacaacggtggcaaggaagcgcaagagaaatttgacaacctgttcaacaacaaagatgaagtgagcgttagcggtgacgaaatcaagaaatatcgtctgtgggcggatcagaaccacaccagcccgtacaccggcaagccgatcccgctgagcaaactgttcaccctggagtacgaaattgagcacatcattccgcaaagccgtatgaagaacgacagcatgagcaacctggtgatcagcgaagcggcggttaacgactttaaggatcgttggctggcgcgtccgctgatcgagaaatatggtggcaccccgattgaacacaacggtcagacctttaccctgctgaaccaagaggaattcgagaagcactgcaacaaaacctttcagaaccaacgtggcaagctgaaaaacctgctgcgtgaggaagtgccggacgatttcgttgaacgtcagatcaacgacaaccgttacattacccgtaaactgggtgaactgctggcgccggcggcgaaagcggatgagggtatcgtgtttaccaccggcagcattaccaacgaactgaaggacaaatggggcttccacaccctgtggcgtgagctgatgaaaccgcgttttgaacgtctggagcagatcctgcaaaagaaactggtggttccggacgaaaaggataccaacaaatttcacttcaacgatccggagccgggtaacccggtggacattaagcgtatcgatcaccgtcatcatgcgctggatgcgctgattgttgcggcgaccacccgtgcgcacattaaatacctgaacagcctgaacagccacaagaaacgtgaaccgtacaagtatctggcgaacaaaggcgtgcgtgattttatccaaccgtggccggacttcaccgcggaagtgaagagccagctgaaacgtctgattgtgagccacaaggttaactgccagtatgatccggaacacccggagaaaagcggtgtgatcagcaagccgaaaaaccgtttcaagaaatgggtgaaccgtgatggcgtttggaagaaagagtaccagtggcaaaaggacaacgaaaactggtgggcgattcgtaagagcatgtttaaagagccgctgggtatgatctacctgaaggaaatcaaagaggtgtctctgaagaaagcgctggagatccaggcggaacgtcaaaaaggtattaaggaccacaccggccgtccgcgtgactacatctatgataagctggcgcgtcaggagattcgtttcctgctggaagacaaatgcggtggcgatatcaagcaggcggaaaaacaaagcagcaccctgaaagatagcaagagcaacccgattaagaaagtgcgtgttgcgtttttcaaagagtacgcggcgagccgtgtgccggttgacaacagcttcacctataagaaaattaaggcgatcccgtacgcggaaaaaatcattaaccgttgggaggaatgggagcaggatggtaaaaacgaaaagggccaaaaattcccgaacgacatcaccaagtggccgattgaatttctgctgaagaaacacctggatgagtataaaaccagcaacggtaacccggacccgaacaccgcgttcaccggtgaaggctacgaggcgctgaccaagaaaaacggtggccagccgatcaagaaagttaccacctatgaaagcaagagcgcgccgatcaagtttaacggtaaaattctggagaccgataaaggtggcaacgtgtttttcgttattgcgaaggataaacacaccggcaagcacctggactggtacaccccgccgctgtatagcaacgaggcggaggaaggtaaggagcgtggcatcattaaccgtctgatcaaccgtgagccgattgcggaagaccaggaagacctggaatatatcaccctggcgccggaagacctggtgtacgttccggaggaagacgaggatattcgtagcatcgactggaacggcaaggataaacaaaaggtgttcgaacgtacctacaagatggttagcagcaccgaaaaagagtgccactttattccgcacatcgtggcgtatccgatcctgaagaccgttgagctgggtaccaacgataagagcgaaaaagcgtgggacggcaaagtggagtacattccgaacaagaaaggtaaactgacccgtaaagatagcggcaccatgatcaaggagaactgcgttaaaattaagctggaccgtctgggtaacatcattaaggtgaacggcaaaccggttaaccactag

SEQ ID NO: 55 - DNA, 3195 bp, Artificial Sequence
  Description of Artificial Sequence: Synthetic polynucleotide
atgtccaatgcccgtccttccatcctgcccgatgatctgatccttggtctcgacatcggtaccaactcggtcggatgggctctcatccactatgccgagagcgaaccgcgacagctcatcgcactcggatcgcgtgtattcgaagcgggcatggacggttcaatcagtcacggcaaggaggagtcacgaaacaagaagcggcgggatgcgcggtcccttcggcgggcgacgtggcgtcgaaagcgtcgaaagcggagggtatacaatctgcttcacgaagcagggctgcttccggacgctgacacgaacgatccggaatcgatcaacgtggctctgacccgactcgatcgggaactcgtttccaagttcgtctcgccgggcgatcatcgcgaggctcagctgatgccgtacctcgccaggcgacgcgccgtggaggagcgcgtagagcctgtcgttttgggtagagcgctctaccacatcgcgcaacggcgaggcttccggtcgaatcggcggacggccatgcgagaagacgaagatctagggcaggtcaaaagcgcgattgcgtcgctgcatcacaagattgttgagtccgaaggagagatccagacgcttggtgggtacttcgcctcactcgatcctcacgaagaacgaatccgtacccgatggacgggtcgtgatatgtacctggaagagttcgataaaatcgttgataggcagattccttaccacgatggccttacgagcgaacgggtcgaggcgctgcgcgctgcgatctttgatcagcgtcccttgcggtcgcaaaatcacctgattggtcgatgcgaactagagcgagatcagaggcgatgctcgattgcccttctggagtatcagcggtttcggttactccaggccgtgaacaatctccgctggctttctgacgaaggtcatgaacgagaactctcgcgggaagaacgtctccgtctggtcagggagcttgagatcaagccggaactcgcattcggaaagattcgcacgcttctcggattgaagcgcggcacaggccggttcaatctggaactcggcggcgagaagcgactcatcggaaatcgcacgaatgcgcagttgcgcgcgctcttcgaggcgcggtgggagacgttcacgaacgacgagcaatcgtcgatcgtgcatgatctgatgagcatccaaaacccgatcgccctgcagcgcagggggcaagtgaggtggggtcttgatggcgagaagagtagctatttcgccaatgacctccttctcgaggatggctacgcgcccctttcgcttcgtgcgattcgaaagctgctgcctcgactcgaggaaggcattccgtattcgacagcgagaaaggagatgtatcctgaatcgttccaatcctcggtcgtgctcgatcggcttccacctcttgctaagacggacctcgaagcgcggaatccgtcgattatgaggacgctctccgaagtacgagcagtggtcaatgccatcgttcgacagtacggaaggcctggactcgttcggattgagctggctcgggatctgaagcagccgaagaggcgacgccaggaaatctcacgacagatgcgggagcgagagggggttcgcgagaaggccaagaagcgcctgcttgataccgagtttggcgggtcgcgagccagccgagccgatatcgaaaagctcatccttgccgacgagtgcgattggacgtgcccgtatacggggcgcggcttcgggatgggcgatctattcggatcaaatcccacgatcgacgtggagcacatccttcccttcagtcgctgtctcgacaattccttcctcaacaagactctctgtgacgtacgcgaaaatcgcctagtgaagcgcaatcggaccccgttcgaagcctatgccggtcagcgcgatcgatgggaagcgatccttgatcggatcaagaacttcaagtcggatccgctgacggtccgtcggaagctggaacgatttctccaagaggaactctcgtcggcgcgagtcgacgagttcagcgagcgcgcgctttccgatacacgatacgcgtcgcgtctggtcgccgacttcatggggttgttgtatgggggacggaacgattccgatgggaagcagcgagttcaggtctccagcggccaagcgacttcgatcctacgtcgtgaatggggtctcaactcgctgctgggcggggaggctcggaagtctcgactcgatcaccgccatcatgcggtcgatgccgtagtcatcgcgttgactgggccacgcgaggtgaaacgactagccgacgctgcaaaacgagcggccgatcaaggaagtcatcgccttttcgaggaggttccgtttccgtggactcatttccgcaccgacgtgaacgagaagattcattgttgcgtgacctctccccgaccgtccaggcggctccgtgggccgcttcacgacgagagcctctattcacgcccgctcccctggtatgacaagaaggggagagagagtcttcggccaaggatccgtaagccgatcgaacagctcaccaagggcgaggttgagcgaatcgcggatccaggcgttcgggacgcggtgaagaccagggccgctgaactcgcgaaagggcaaggaggcagtggggatctcagtaagctcttctccgacccgagccacgctccgtttctgcgaaaccgtgatggttcgaccaccccgattcggcgcgtccggattaccgcgaaggtcaagcaggccacgccgatcggagaaggtgttcgtcaacgtcatgtcgcgcccggctcgaatcatcacatggcgatcgttgcaattctggacgagaaggggaatgagaagcgctgggaaggtcatgtcgtcacgatgctggaggccgtgctccggaaggggcgtggggagccggtgatccaacgggattggggaaaggggcaaaagttcaagttttcgcttcgatcgggagactgcatctggaattgcgacaccgggcggattatgcatgtcaaggcggtttcagcgggtgtcgtggaaggcctcgaagtgaacgatgcccggacagcggttgatgtgagaagagccggcgtcgttggagggcgctatacggcaagcccagagcgacttcgaaaagacgctttcgttcgctgtgtcgtggacccactcgggaaggtcataccatccaatgagtga

SEQ ID NO: 56 - DNA, 3195 bp, Artificial Sequence
  Description of Artificial Sequence: Synthetic polynucleotide
atgagcaacgcgcgtccgagcattctgccggacgatctgatcctgggtctggacattggcaccaacagcgtgggttgggcgctgattcactacgcggagagcgaaccgcgtcaactgatcgcgctgggtagccgtgttttcgaggcgggtatggatggcagcatcagccacggcaaagaggagagccgtaacaagaaacgtcgtgatgcgcgtagcctgcgtcgtgcgacctggcgtcgtaagcgtcgtaaacgtcgtgtgtataacctgctgcatgaagcgggtctgctgccggacgcggataccaacgacccggagagcattaacgttgcgctgacccgtctggatcgtgaactggttagcaaatttgttagcccgggtgaccaccgtgaagcgcagctgatgccgtatctggcgcgtcgtcgtgcggtggaggaacgtgttgaaccggtggttctgggtcgtgcgctgtatcacatcgcgcagcgtcgtggcttccgtagcaaccgtcgtaccgcgatgcgtgaggacgaagatctgggtcaagtgaagagcgcgatcgcgagcctgcaccacaaaattgttgagagcgaaggcgagatccagaccctgggtggctactttgcgagcctggatccgcacgaggaacgtatccgtacccgttggaccggtcgtgacatgtacctggaggaattcgacaagatcgtggatcgtcaaattccgtatcacgatggcctgaccagcgaacgtgttgaggcgctgcgtgcggcgatttttgaccagcgtccgctgcgtagccaaaaccacctgatcggtcgttgcgaactggagcgtgatcagcgtcgttgcagcatcgcgctgctggagtatcagcgtttccgtctgctgcaagcggtgaacaacctgcgttggctgagcgacgaaggccacgaacgtgagctgagccgtgaggaacgtctgcgtctggttcgtgaactggagattaagccggagctggcgtttggtaaaatccgtaccctgctgggtctgaagcgtggtaccggccgtttcaacctggaactgggtggcgagaaacgtctgattggtaaccgtaccaacgcgcagctgcgtgcgctgtttgaagcgcgttgggagaccttcaccaacgacgaacagagcagcatcgtgcacgatctgatgagcatccaaaacccgattgcgctgcagcgtcgtggtcaagttcgttggggtctggatggcgagaagagcagctactttgcgaacgacctgctgctggaagatggttatgcgccgctgagcctgcgtgcgattcgtaagctgctgccgcgtctggaggaaggcatcccgtacagcaccgcgcgtaaagaaatgtatccggagagcttccagagcagcgtggttctggaccgtctgccgccgctggcgaaaaccgatctggaggcgcgtaacccgagcattatgcgtaccctgagcgaagtgcgtgcggtggttaacgcgattgttcgtcagtacggtcgtccgggtctggtgcgtattgagctggcgcgtgacctgaagcaaccgaaacgtcgtcgtcaggaaatcagccgtcaaatgcgtgaacgtgagggtgttcgtgagaaggcgaagaaacgtctgctggataccgaatttggtggcagccgtgcgagccgtgcggacattgagaaactgattctggcggacgaatgcgattggacctgcccgtacaccggtcgtggctttggtatgggcgacctgttcggtagcaacccgaccatcgatgtggagcacattctgccgtttagccgttgcctggacaacagcttcctgaacaagaccctgtgcgatgtgcgtgaaaaccgtctggttaaacgtaaccgtaccccgtttgaggcgtatgcgggtcaacgtgaccgttgggaagcgatcctggatcgtattaagaacttcaaaagcgatccgctgaccgtgcgtcgtaagctggagcgttttctgcaggaagagctgagcagcgcgcgtgttgacgaattcagcgagcgtgcgctgagcgatacccgttacgcgagccgtctggttgcggacttcatgggtctgctgtatggtggccgtaacgacagcgatggcaagcagcgtgtgcaagttagcagcggccaagcgaccagcattctgcgtcgtgagtggggcctgaacagcctgctgggtggcgaagcgcgtaaaagccgtctggaccaccgtcaccatgcggtggatgcggtggttatcgcgctgaccggtccgcgtgaggttaaacgtctggcggatgcggcgaaacgtgcggcggatcagggtagccaccgtctgttcgaggaagtgccgtttccgtggacccacttccgtaccgacgtgaacgagaagattcattgctgcgttaccagcccgcgtccgagccgtcgtctgcgtggtccgctgcacgatgaaagcctgtacagccgtccgctgccgtggtatgacaagaaaggccgtgagagcctgcgtccgcgtatccgtaagccgattgaacaactgaccaaaggtgaagttgaacgtattgcggacccgggcgtgcgtgatgcggttaagacccgtgcggcggagctggcgaagggtcagggtggcagcggcgacctgagcaaactgtttagcgatccgagccacgcgccgttcctgcgtaaccgtgacggtagcaccaccccgatccgtcgtgtgcgtattaccgcgaaggttaaacaggcgaccccgattggtgaaggcgtgcgtcaacgtcatgttgcgccgggtagcaaccaccacatggcgatcgtggcgattctggatgaaaagggtaacgagaaacgttgggaaggccacgtggttaccatgctggaggcggtgctgcgtaagggtcgtggcgaaccggttatccagcgtgactggggtaaaggccaaaagttcaaatttagcctgcgtagcggtgactgcatttggaactgcgataccggccgtatcatgcacgtgaaagcggttagcgcgggtgtggttgaaggcctggaagtgaacgacgcgcgtaccgcggtggatgttcgtcgtgcgggtgtggttggtggccgttacaccgcgagcccggagcgtctgcgtaaggacgcgttcgtgcgttgcgtggttgatccgctgggcaaagttatcccgagcaacgaatag

SEQ ID NO: 57 - DNA, 3075 bp, Artificial Sequence
  Description of Artificial Sequence: Synthetic polynucleotide
atgacatatattttgggtttagacctcggcatttcatcggtcggctttgccggcattgatcataatggggataatattcttttcgcaaatgcccatgtatttgataaggcagaggttgccaaaaccggcgcatcgctggctgaaccacggcgtaatgcccgcctgacccgccgccgcatcgaacggaaagcccggcgcaaatcacgtattaaaaatttatttgataaatatggcttggatgtggaggcgattgaccgcccgccttccccggatcgtcaatcggtatgggatttgcgacgggttggcttgtcaaaaaaattaaactcgggccaatgggcacgtgcgttatttcatttggccaaaaaccgtggctttcaatccaaccgaaaggataaggcagacggggtcggcactggtaaatcggataccgataacggccggatgctgtcggcgatttccgatttgaaaaaaaatctggcggagagcgaccatgaaacaatcggatcttatttatccacgctggataaaaaacgcaacggggatgatgattattccaaaaccgtgcatcgggatatgatccgggatgaggtttccttactatttcaacggcaacgatcctttgataacccgcatgccggaacggagttggaacaggcgttttgtaaggttgccttttatcaacgcccattgcagtccaccatcgaattaatcggtaattgcagtattttcccggatgaaaaacgggcgccgaaacatgcctattcaagtgaagaatttttggcctggagccggctgaataatttacgcttactcaccccgtccggcaaaaaaaaggaattgacgacaggtcaaaaagaaaaggccatagagctgaccaagcagtataaaaaaggcgtaacctttgcccgcctgcgccgtgcattggacatcgatgatcaatatcggtttaatctatgccattaccgcaataccatggatggcccatcggattgggacacaatccgggataaatcggaaaaacaggttttaatccaatttccgggctatcacgccatgcgggatcaattatccgacctcggtgcggatgatatccattttaccgaattattggccaaccgggatcaatatgatgacaccatccaaattttgagtttttatgaggatgaggccgatatcctgtcccgtctatcggacctgggccatttgcctgaagtcatcgaaaaactaaaatatcttgatttttcccgaaccatcgatctgtcattaaaggcggtgaaacagatcctgccttatatgaaaaaggggtatgattatgccacggcaagggatatggccgggcttaagccaaaaaatacaaaaagcgggaataaaaaactgttatccccgtttgattcgacaaaaaatccggttgttgaccggtgccttgcccaatccagaaaggttgttaatgcggttattcgtcgccatggacttcccgattatattcatatcgaattatcacgtgacctgggccgatcaaaaaaagaacgggataaaattgatcgccgtattgaaaaaaatcgccggtataaagaagatctgcgtcagcatgccgccgaattattggatcgggagccaagcggggaagaatttttaaaataccgcctttggaaagaacaagacggtatatgcccctattccggcagttatatcgaaccggatgaatgggcatcgcccacggcggtacaaattgatcatatcctgcccttttcaagatcctatgacaatagttacatgaataaggtgctttgcacggccagcgcaaatcaggaaaaggggaataaaaccccgtatgaatgctggggtcagatggatgatctatggcccgcgattatggcacaggcggataaactgcctaagaaaaaacgggatcgtatattaaacaaacattttaatgaacgggaacaggaattcaaaacccgtcatttaaatgatacccgctatattgcccgccagcttcgccaaaatatttctgaacaactggatctgggggatggcaatcgggtgcgtgtgcgcaatggatatatcacatcctttttacgtgggatatggggattacaggataaaacccgtgacaatgaccgccatcatgccattgatgcgattattgttgcctgcaccaccgaaggtattatgcaacaggtcacccaatggaataaatatgatgcccgacgcaaggataaagaaccctatttccccaaaccatgggatggttttcgatccgatgtgtgggatgcctatcatgcggtgtttgtttcccgcctacccgaccggtcggccaccggggcgatgcataaagaaacggtacgaagcctgcgcaccgatgatgatggtaatgatgtcgtggtccaacgtatcccgattaccgatctttccaaggccaagttagaggatatcgttgataaagatacccgcaacaccaggctgtacaatacccttaaaacccggatggaaaaacatgggtataaggcggataaggcatttgccaaaccaatctacatgcccaccaactcggataaacaaggcccgccgattaaacgggtgcgtattgtcaccaataagcaaaaggatattgtcttgcccaaacgcgggggcggagtcgccgatcgggcaaatatggtccgggtggatgtctttgaaaaaggggggaattttttcctttgcccggtatataccgatcaaattatgcggggcgaactgccgatgcgcctggtaaaggccagtaaagacgaatccgaatggccggaaattaccgatgagtatgattttaaattcagcctgtataaaaatgactatgtcaaaataaagaaaaaatccaaaggagagattgtagaattagaggggtattataatggtactgatcgtgcaacggccagtataagcctacgcattcatgacaatgatcaggatgtcggtaaaaacggcatgatcagaggcattggcgtttaccgactgttatcctttgaaaaatatactgtgagttactttgggcaattatcacgggtaaaccaagggggtcgacctggcgtggcgtag

SEQ ID NO: 58 - DNA, 3075 bp, Artificial Sequence
  Description of Artificial Sequence: Synthetic polynucleotide
atgacctacatcctgggtctggacctgggcattagcagcgttggtttcgcgggcatcgatcacaacggtgacaacattctgttcgcgaacgcgcacgtgtttgataaggcggaagttgcgaagaccggtgcgagcctggcggaaccgcgtcgtaacgcgcgtctgacccgtcgtcgtatcgaacgtaaagcgcgtcgtaagagccgtatcaagaacctgtttgataagtacggtctggacgttgaagcgattgatcgtccgccgagcccggaccgtcagagcgtgtgggatctgcgtcgtgttggtctgagcaagaaactgaacagcggccagtgggcgcgtgcgctgttccacctggcgaaaaaccgtggttttcaaagcaaccgtaaagataaagcggatggtgtgggtaccggcaagagcgacaccgataacggccgtatgctgagcgcgatcagcgacctgaagaaaaacctggcggaaagcgatcacgagaccattggtagctacctgagcaccctggacaagaaacgtaacggcgacgatgactatagcaaaaccgtgcaccgtgatatgatccgtgacgaagttagcctgctgttccagcgtcaacgtagctttgacaacccgcacgcgggtaccgagctggaacaggcgttctgcaaggtggcgttttaccagcgtccgctgcaaagcaccatcgaactgattggcaactgcagcatcttcccggacgagaagcgtgcgccgaaacacgcgtatagcagcgaggaatttctggcgtggagccgtctgaacaacctgcgtctgctgaccccgagcggtaagaaaaaggagctgaccaccggccagaaagaaaaggcgatcgagctgaccaagcaatacaaaaagggtgttaccttcgcgcgtctgcgtcgtgcgctggacattgatgaccagtaccgttttaacctgtgccactatcgtaacaccatggacggcccgagcgactgggataccatccgtgataaaagcgaaaagcaggtgctgattcaattcccgggttatcacgcgatgcgtgatcaactgagcgacctgggcgcggatgacatccacttcaccgagctgctggcgaaccgtgaccagtacgatgacaccatccaaattctgagcttttatgaggatgaagcggacatcctgagccgtctgagcgatctgggtcacctgccggaagttattgagaaactgaagtacctggacttcagccgtaccatcgatctgagcctgaaagcggtgaagcagattctgccgtatatgaaaaagggctacgactatgcgaccgcgcgtgatatggcgggtctgaaaccgaagaacaccaaaagcggcaacaaaaagctgctgagcccgtttgacagcaccaaaaacccggtggttgatcgttgcctggcgcaaagccgtaaggtggttaacgcggttatccgtcgtcacggtctgccggactacatccacattgaactgagccgtgatctgggccgtagcaaaaaggagcgtgataagatcgaccgtcgtattgaaaagaaccgtcgttacaaagaggacctgcgtcagcacgcggcggaactgctggatcgtgagccgagcggcgaggaattcctgaagtatcgtctgtggaaagagcaggacggtatctgcccgtacagcggcagctatattgagccggatgagtgggcgagcccgaccgcggttcaaatcgaccacattctgccgtttagccgtagctacgataacagctatatgaacaaagtgctgtgcaccgcgagcgcgaaccaagaaaagggtaacaagaccccgtacgagtgctggggccagatggatgacctgtggccggcgatcatggcgcaagcggacaagctgccgaaaaagaaacgtgatcgtattctgaacaaacacttcaacgagcgtgaacaggagtttaagacccgtcacctgaacgacacccgttacatcgcgcgtcagctgcgtcaaaacattagcgaacaactggatctgggtgacggcaaccgtgttcgtgtgcgtaacggttatatcaccagcttcctgcgtggtatttggggcctgcaggacaaaacccgtgacaacgatcgtcaccacgcgatcgatgcgatcattgtggcgtgcaccaccgaaggtattatgcagcaagttacccaatggaacaaatacgacgcgcgtcgtaaagataaggagccgtatttcccgaagccgtgggacggctttcgtagcgatgtttgggacgcgtaccacgcggttttcgttagccgtctgccggatcgtagcgcgaccggtgcgatgcacaaggagaccgtgcgtagcctgcgtaccgatgacgatggcaacgacgtggttgtgcagcgtatcccgattaccgacctgagcaaagcgaagctggaagatatcgtggacaaagatacccgtaacacccgtctgtataacaccctgaagacccgtatggagaaacacggttacaaagcggacaaggcgttcgcgaagccgatctatatgccgaccaacagcgataaacagggtccgccgatcaagcgtgtgcgtattgttaccaacaaacaaaaggacattgtgctgccgaaacgtggtggcggtgttgcggaccgtgcgaacatggttcgtgtggatgtttttgaaaagggcggtaacttctttctgtgcccggtttacaccgaccagatcatgcgtggtgagctgccgatgcgtctggtgaaagcgagcaaggatgaaagcgagtggccggaaattaccgatgagtatgacttcaagtttagcctgtacaaaaacgactatgtgaagatcaagaaaaagagcaaaggtgaaattgttgaactggagggttactataacggcaccgatcgtgcgaccgcgagcatcagcctgcgtattcacgacaacgatcaggacgtgggtaaaaacggcatgatccgtggtattggcgtttaccgtctgctgagcttcgagaagtacaccgtgagctattttggtcagctgagccgtgtgaaccaaggcggtcgtccgggcgttgcgtag

SEQ ID NO: 59 - DNA, 2277 bp, Artificial Sequence
  Description of Artificial Sequence: Synthetic polynucleotide
atgtctgttcgcgcaatccgtgcccgcatcgcctgcgatcggactgtactcgatcacctctggcgcacccattgtgtctttcacgagcggctgccgattgtgctgggctggcttttccgcatgcgacgaggcgaatgcggcgagactgatgccgagcgactcctttaccagcgcgtcggcaagttcattactggctattccgcccagaacgctgactacctaatgaacgcggtcagcctgaaaggctggaagccggccaccgccaagaaatacaagattaagaccgacgacgacaacggtcagtcggtccagatcagcggcgagtcgtgggccgatgaggctgctgccctttcggcccaaggaaagctactcttcgacaagaacgtggtttcgggtggcctgcccggatgtatgagacagatgctcaatcgagaatccgtcgccattatcagcggccacgacgaactgctgtccaagtggaacacagaccacaccaagtggctcggcgagaaagcccaatgggaagccgttcctgaacacacgctctacctcgcgcttcgcaaaaagttcgagtcctttgaacaagccgttggcggtaaggcgaccaagaggcgagggcgttggcaccgctatctcgactggttgcgcgccaatcctgatttggccgcttggcgcggcgggcccgcgattgtcgacgaactgtcacccgctgcgcaagaacgtatccgcaaggccaaaccatggaagaaacggtccgccgaggcggaagagttctggaagatcaatcccgagcttgcctcgctcgacaagctccacggttactatgagcgcgagttcgttcgccggcgcaagaacaaacgcaaccccgatggttttgatcaccggccaacgttcaccatgcccgaccggattcggcacccgcgctggtttgttttcaacgcaccgcagacgaatccatccggatatcgccatctgcgcttgcctcaaggcgccaaagaaatcggcgccgtgcagctccagctaatcaccggcgggcgcgaaggcgagggcgtgtacccaacgcaatgggtcgacgtgacgtatcgcgccgacccgcgcttggcgctgttccgccggtcgcaagtgtcgaccacagtcaatcgggggaaagcgaaaggacagacaaagatcaaggaaggctacgagttctttgaccggcatctgagccaatggcggtccgcggagatcagcggcgtcaaactgatcttccgcgacatccggcttaatgacgacggctcactgaagtcggctattccctacctggtgttcgcgtgcagcattgatgatcttccacttactgagcgggccaagaagatcgaatggtctgagacgggcgagacgacaaagaccgggaagaaacgaaaatcccgcacgctgcccgacgggctcatcgcgtgtgccgtggatctggggttacgcaacgtcggctttgctacactctgtgtctttgaacacggaaagtcacgcgtcctgcggtcgcgcaatatctggctggatgatgagggtggtggccccgacctgggacacatcggccagcacaaacgacagatcaagcgactgcgccgcaagcgcggcaagccggtcaagggcgaactctcacacgtggagttgcaggaccacattacacacatgggagaagaccgtttcaagaaggcagcgcgcggcatcatcaacttcgcttggaacgtggacggtgcggtcgacgaagccacgggcgagccattccctcgcgcggatgcgattgttctcgaaaagctcgaaggtttcatcccggatgccgaaaaagagcgcgggatcaaccgcagtcttgccgcatggaaccgcggccaactggtaacacgcctcgaggagatggcgattgacgccggctacaaaggtcgtgttttcaaggtccatccggccggtacgtcgcaggtgtgttcccgttgcggcgcgctcggacggcgttactcaatcacccgcgacaatgccgcgcacacgcccgacattcgctttggctgggtcgaaaagctctttgcgtgcccgtgcggttatcgcgccaactccgaccacaatgcctccgtcaaccttcagcggaaattccagatgggcgacgaggcagtaaaggcgttctcctcgtggcgaaatcaaaccgaagcccaacggcaacacgcccttgagagcttggacgcctcgctccgggatggcttgcggaaaatgcacgggttgccgtttccgcctcttgataatcccttttga

SEQ ID NO: 60 - DNA, 2277 bp, Artificial Sequence
  Description of Artificial Sequence: Synthetic polynucleotide
atgagcgttcgtgcgatccgtgcgcgtattgcgtgcgatcgtaccgtgctggaccacctgtggcgtacccactgcgttttccacgaacgtctgccgattgtgctgggctggctgtttcgtatgcgtcgtggcgagtgcggtgaaaccgatgcggagcgtctgctgtaccagcgtgttggcaaattcatcaccggttacagcgcgcaaaacgcggactatctgatgaacgcggtgagcctgaagggttggaaaccggcgaccgcgaagaaatataagattaaaaccgacgatgacaacggccagagcgttcaaatcagcggtgaaagctgggcggatgaggctgcggcgctgagcgcgcagggtaaactgctgtttgacaagaacgtggttagcggtggcctgccgggttgcatgcgtcaaatgctgaaccgtgaaagcgtggcgatcattagcggccacgatgagctgctgagcaagtggaacaccgaccacaccaaatggctgggtgaaaaggcgcagtgggaagcggttccggagcacaccctgtacctggcgctgcgtaagaaattcgagagctttgaacaagcggtgggtggcaaggcgaccaaacgtcgtggtcgttggcaccgttatctggattggctgcgtgcgaacccggacctggcggcgtggcgtggtggcccggcgattgtggatgagctgagcccggcggcgcaggagcgtatccgtaaggcgaaaccgtggaagaaacgtagcgcggaagcggaggaattctggaaaattaacccggagctggcgagcctggataagctgcacggctactatgagcgtgaatttgttcgtcgtcgtaagaacaaacgtaacccggatggtttcgaccaccgtccgacctttaccatgccggaccgtatccgtcacccgcgttggttcgtgtttaacgcgccgcagaccaacccgagcggttaccgtcacctgcgtctgccgcaaggcgcgaaagagatcggtgcggttcagctgcaactgattaccggtggccgtgagggcgaaggtgtgtacccgacccagtgggtggatgttacctatcgtgcggacccgcgtctggcgctgttccgtcgtagccaggtgagcaccaccgttaaccgtggcaaggcgaaaggtcaaaccaagattaaagagggttacgaattctttgatcgtcacctgagccaatggcgtagcgcggaaatcagcggcgttaaactgatcttccgtgacattcgtctgaacgatgacggtagcctgaagagcgcgatcccgtatctggtgtttgcgtgcagcattgatgacctgccgctgaccgagcgtgcgaagaaaattgagtggagcgaaaccggcgaaaccaccaaaaccggtaagaaacgtaaaagccgtaccctgccggatggcctgattgcgtgcgcggtggacctgggcctgcgtaacgttggtttcgcgaccctgtgcgtgtttgaacacggcaagagccgtgtgctgcgtagccgtaacatttggctggatgatgagggtggcggtccggatctgggtcacatcggtcagcacaaacgtcaaattaagcgtctgcgtcgtaagcgtggcaaaccggttaagggtgaactgagccacgtggagctgcaggatcacatcacccacatgggcgaggaccgtttcaagaaagcggcgcgtggtatcattaactttgcgtggaacgtggatggtgcggttgatgaagcgaccggcgagccgttcccgcgtgcggatgcgatcgttctggaaaaactggagggctttattccggacgcggagaaggaacgtggtatcaaccgtagcctggcggcgtggaaccgtggtcagctggttacccgtctggaggaaatggcgatcgacgcgggctacaaaggtcgtgtgttcaaggttcatccggcgggtaccagccaggtttgcagccgttgcggtgcgctgggtcgtcgttatagcattacccgtgataacgcggcgcacaccccggacatccgtttcggctgggtggaaaaactgtttgcgtgcccgtgcggttaccgtgcgaacagcgatcacaacgcgagcgttaacctgcagcgtaaattccaaatgggtgacgaggcggtgaaggcgtttagcagctggcgtaaccagaccgaagcgcagcgtcaacatgcgctggagagcctggatgcgagcctgcgtgatggcctgcgtaagatgcatggtctgccgttcccgccgctggacaacccgttttag

SEQ ID NO: 61 - DNA, 36 bp, Artificial Sequence
  Description of Artificial Sequence: Synthetic oligonucleotide
gtttaagagaataaagaaatttctactattgtagat

SEQ ID NO: 62 - protein, 18 aa, Artificial Sequence
  Description of Artificial Sequence: Synthetic peptide
INILSIDRGERHLAYWTL

SEQ ID NO: 63 - protein, 13 aa, Artificial Sequence
  Description of Artificial Sequence: Synthetic peptide
NAIIVFEDLNYGF

SEQ ID NO: 64 - protein, 16 aa, Artificial Sequence
  Description of Artificial Sequence: Synthetic peptide
EPANADSNGAYNIGIK

SEQ ID NO: 65 - skipped

SEQ ID NO: 66 - DNA, 25 bp, Artificial Sequence
  Description of Artificial Sequence: Synthetic oligonucleotide
catcgaaagttaggaactaaaaggc

SEQ ID NO: 67 - protein, 22 aa, Artificial Sequence
  Description of Artificial Sequence: Synthetic peptide
NYPILGVDVGEYGLAYCLILVD

SEQ ID NO: 68 - protein, 24 aa, Artificial Sequence
  Description of Artificial Sequence: Synthetic peptide
HVVLITDQGASSVYEYQISNFETR

SEQ ID NO: 69 - protein, 16 aa, Artificial Sequence
  Description of Artificial Sequence: Synthetic peptide
FVADADIQAAFMMALR

SEQ ID NO: 70 - DNA, 36 bp, Artificial Sequence
  Description of Artificial Sequence: Synthetic oligonucleotide
ctctagggctaccccaaaatttctactattgtagat

SEQ ID NO: 71 - protein, 18 aa, Artificial Sequence
  Description of Artificial Sequence: Synthetic peptide
IKIIGLDRGERHLLYLSL

SEQ ID NO: 72 - protein, 13 aa, Artificial Sequence
  Description of Artificial Sequence: Synthetic peptide
NSIVVLEDLNAGF

SEQ ID NO: 73 - protein, 16 aa, Artificial Sequence
  Description of Artificial Sequence: Synthetic peptide
APKDADANGAYHIALK

SEQ ID NO: 74 - DNA, 36 bp, Artificial Sequence
  Description of Artificial Sequence: Synthetic oligonucleotide
cgcaataagacctatacaatttctacttttgtagat

SEQ ID NO: 75 - protein, 18 aa, Artificial Sequence
  Description of Artificial Sequence: Synthetic peptide
VCFLGIDRGEKHLAYYSI

SEQ ID NO: 76 - protein, 13 aa, Artificial Sequence
  Description of Artificial Sequence: Synthetic peptide
NAFIVLEDLNVGF

SEQ ID NO: 77 - protein, 16 aa, Artificial Sequence
  Description of Artificial Sequence: Synthetic peptide
LPISGDANGAYNIARK

SEQ ID NO: 78 - DNA, 25 bp, Artificial Sequence
  Description of Artificial Sequence: Synthetic oligonucleotide
ccccgaaaaatggggatgaaaaggc

SEQ ID NO: 79 - DNA, 64 bp, Artificial Sequence
  Description of Artificial Sequence: Synthetic oligonucleotide
ctaggtgtatgttgttccgatgttatcgtgagatacattttagccttctcaacatacaaataat

SEQ ID NO: 80 - protein, 22 aa, Artificial Sequence
  Description of Artificial Sequence: Synthetic peptide
FSRYLGLDLGEFGVAWAVLGIK

SEQ ID NO: 81 - protein, 23 aa, Artificial Sequence
  Description of Artificial Sequence: Synthetic peptide
HSLVLRYGAKMVFERQVDAFQTG

SEQ ID NO: 82 - protein, 15 aa, Artificial Sequence
  Description of Artificial Sequence: Synthetic peptide
RTYDADKQAAVNIAM

SEQ ID NO: 83 - DNA, 25 bp, Artificial Sequence
  Description of Artificial Sequence: Synthetic oligonucleotide
cctcgtgatacggggagagaaaggc

SEQ ID NO: 84 - DNA, 64 bp, Artificial Sequence
  Description of Artificial Sequence: Synthetic oligonucleotide
ctccataccgggtttcccggcgcgtgcgccgcaccgccgatcgcctttccccggttcctcttgt

SEQ ID NO: 85 - protein, 22 aa, Artificial Sequence
  Description of Artificial Sequence: Synthetic peptide
YSYLLGLDVGEYGIAYCLLEPE

SEQ ID NO: 86 - protein, 23 aa, Artificial Sequence
  Description of Artificial Sequence: Synthetic peptide
HDLTVRYDARPVYEFNISNFESG

SEQ ID NO: 87 - protein, 15 aa, Artificial Sequence
  Description of Artificial Sequence: Synthetic peptide
HTADCDVQAALIVAV

SEQ ID NO: 88 - DNA, 36 bp, Artificial Sequence
  Description of Artificial Sequence: Synthetic oligonucleotide
ctcaaataaacctatcaaatttctactttcgtagat

SEQ ID NO: 89 - protein, 18 aa, Artificial Sequence
  Description of Artificial Sequence: Synthetic peptide
VNIIGIDRGEKHLAYYSV

SEQ ID NO: 90 - protein, 13 aa, Artificial Sequence
  Description of Artificial Sequence: Synthetic peptide
NAIVVFEDLNLGF

SEQ ID NO: 91 - protein, 16 aa, Artificial Sequence
  Description of Artificial Sequence: Synthetic peptide
FQFNGDANGAYNIARK

SEQ ID NO: 92 - DNA, 36 bp, Artificial Sequence
  Description of Artificial Sequence: Synthetic oligonucleotide
gctgtctttacctttcaaaacaggggcagttacagc

SEQ ID NO: 93 - protein, 6 aa, Artificial Sequence
  Description of Artificial Sequence: Synthetic peptide
  SITE (1)..(1): /note=“N terminus is linked to a peptide that contains a glutamic acid followed by a gap of unknown length”
  MOD_RES (2)..(5): Any amino acid
RXXXXH

SEQ ID NO: 94 - protein, 6 aa, Artificial Sequence
  Description of Artificial Sequence: Synthetic peptide
  SITE (1)..(1): /note=“N terminus is linked to a peptide that contains a glutamic acid followed by a gap of unknown length”
RNYYTH

SEQ ID NO: 95 - protein, 6 aa, Artificial Sequence
  Description of Artificial Sequence: Synthetic peptide
  SITE (1)..(1): /note=“N terminus is linked to a peptide that contains a glutamic acid followed by a gap of unknown length”
RNKFSH

SEQ ID NO: 96 - DNA, 36 bp, Artificial Sequence
  Description of Artificial Sequence: Synthetic oligonucleotide
gttgtagctgccctgattttgcagggtacacacaac

SEQ ID NO: 97 - protein, 6 aa, Artificial Sequence
  Description of Artificial Sequence: Synthetic peptide
  SITE (1)..(1): /note=“N terminus is linked to a peptide that contains a glutamic acid followed by a gap of unknown length”
RNNFSH

SEQ ID NO: 98 - DNA, 36 bp, Artificial Sequence
  Description of Artificial Sequence: Synthetic oligonucleotide
gttgttgctgttctgattttgcagggtagatacaac

SEQ ID NO: 99 - protein, 6 aa, Artificial Sequence
  Description of Artificial Sequence: Synthetic peptide
  SITE (1)..(1): /note=“N terminus is linked to a peptide that contains a glutamic acid followed by a gap of unknown length”
RNDYSH

SEQ ID NO: 100 - protein, 6 aa, Artificial Sequence
  Description of Artificial Sequence: Synthetic peptide
  SITE (1)..(1): /note=“N terminus is linked to a peptide that contains a glutamic acid followed by a gap of unknown length”
RNSFSH

SEQ ID NO: 101 - DNA, 36 bp, Artificial Sequence
  Description of Artificial Sequence: Synthetic oligonucleotide
ctcgcagacgacgcccaagtggagggcgactgcacc

SEQ ID NO: 102 - protein, 6 aa, Artificial Sequence
  Description of Artificial Sequence: Synthetic peptide
  SITE (1)..(1): /note=“N terminus is linked to a peptide that contains a glutamic acid followed by a gap of unknown length”
RNHFAH

SEQ ID NO: 103 - DNA, 36 bp, Artificial Sequence
  Description of Artificial Sequence: Synthetic oligonucleotide
gctgttgaagcctttattttgaaaggtaggtgcagc

SEQ ID NO: 104 - protein, 6 aa, Artificial Sequence
  Description of Artificial Sequence: Synthetic peptide
  SITE (1)..(1): /note=“N terminus is linked to a peptide that contains a glutamic acid followed by a gap of unknown length”
CNYYTH

SEQ ID NO: 105 - protein, 6 aa, Artificial Sequence
  Description of Artificial Sequence: Synthetic peptide
  SITE (1)..(1): /note=“N terminus is linked to a peptide that contains a glutamic acid followed by a gap of unknown length”
RSILSH

SEQ ID NO: 106 - DNA, 36 bp, Artificial Sequence
  Description of Artificial Sequence: Synthetic oligonucleotide
gttgttgtagcctctgatttgaatggtaggaacaac

SEQ ID NO: 107 - protein, 6 aa, Artificial Sequence
  Description of Artificial Sequence: Synthetic peptide
  SITE (1)..(1): /note=“N terminus is linked to a peptide that contains a glutamic acid followed by a gap of unknown length”
RNFFTH

SEQ ID NO: 108 - protein, 6 aa, Artificial Sequence
  Description of Artificial Sequence: Synthetic peptide
  SITE (1)..(1): /note=“N terminus is linked to a peptide that contains a glutamic acid followed by a gap of unknown length”
RNSAAH

SEQ ID NO: 109 - DNA, 36 bp, Artificial Sequence
  Description of Artificial Sequence: Synthetic oligonucleotide
gttgtttatacccttcaaaaaaagagcagtgacaac

SEQ ID NO: 110 - protein, 6 aa, Artificial Sequence
  Description of Artificial Sequence: Synthetic peptide
  SITE (1)..(1): /note=“N terminus is linked to a peptide that contains a glutamic acid followed by a gap of unknown length”
RNINSH

SEQ ID NO: 111 - protein, 6 aa, Artificial Sequence
  Description of Artificial Sequence: Synthetic peptide
  SITE (1)..(1): /note=“N terminus is linked to a peptide that contains a glutamic acid followed by a gap of unknown length”
RNKAFH

SEQ ID NO: 112 - DNA, 36 bp, Artificial Sequence
  Description of Artificial Sequence: Synthetic oligonucleotide
gttgtttttaccccacaaatcaggagcagttacaac

SEQ ID NO: 113 - protein, 6 aa, Artificial Sequence
  Description of Artificial Sequence: Synthetic peptide
  SITE (1)..(1): /note=“N terminus is linked to a peptide that contains a glutamic acid followed by a gap of unknown length”
RNCFSH

SEQ ID NO: 114 - DNA, 100 bp, Artificial Sequence
  Description of Artificial Sequence: Synthetic oligonucleotide
agtgtatcagagcgactgttccgcttatcacggtaagggaacaaaaccgcgcagggcaatgtcacagacaccccttcaacgcctcccagtggggcgtttt

SEQ ID NO: 115 - DNA, 36 bp, Artificial Sequence
  Description of Artificial Sequence: Synthetic oligonucleotide
gccgtggtttggccggaatggtcgctctgatacact

SEQ ID NO: 116 - protein, 18 aa, Artificial Sequence
  Description of Artificial Sequence: Synthetic peptide
RYTLGLDLGVSSIGWAMI

SEQ ID NO: 117 - protein, 13 aa, Artificial Sequence
  Description of Artificial Sequence: Synthetic peptide
PAHIRIELARDLK

SEQ ID NO: 118 - protein, 16 aa, Artificial Sequence
  Description of Artificial Sequence: Synthetic peptide
RHHAVDALVVAFTSQG

SEQ ID NO: 119 - DNA, 105 bp, Artificial Sequence
  Description of Artificial Sequence: Synthetic oligonucleotide
tttatctttgaattacgcttttatcaaaccataataaggattattccgtagaaaactaatctgcagccccatttcacgaaagtgagatcggggttgttgtttttt

SEQ ID NO: 120 - DNA, 36 bp, Artificial Sequence
  Description of Artificial Sequence: Synthetic oligonucleotide
gttgtggtttgattaaaagcgtggattaacgatatt

SEQ ID NO: 121 - protein, 18 aa, Artificial Sequence
  Description of Artificial Sequence: Synthetic peptide
TKILGLDIGTNSVGGALI

SEQ ID NO: 122 - protein, 13 aa, Artificial Sequence
  Description of Artificial Sequence: Synthetic peptide
PDEIHIEMSRELK

SEQ ID NO: 123 - protein, 16 aa, Artificial Sequence
  Description of Artificial Sequence: Synthetic peptide
RHHALDALIVAATTRA

SEQ ID NO: 124 - DNA, 137 bp, Artificial Sequence
  Description of Artificial Sequence: Synthetic oligonucleotide
agtgtatctgagcgatggttgtcgcctatctcagtaaaggacttccatccgcagggccgtacatcccgattctccctccagcagggagagcactctgtacacccttcaggggtggcttcttagaagccgcccctttt

SEQ ID NO: 125 - DNA, 36 bp, Artificial Sequence
  Description of Artificial Sequence: Synthetic oligonucleotide
gctggggttcgtcggcagccatcgctcagatacact

SEQ ID NO: 126 - protein, 19 aa, Artificial Sequence
  Description of Artificial Sequence: Synthetic peptide
DDLILGLDIGTNSVGWALI

SEQ ID NO: 127 - protein, 13 aa, Artificial Sequence
  Description of Artificial Sequence: Synthetic peptide
PGLVRIELARDLK

SEQ ID NO: 128 - protein, 16 aa, Artificial Sequence
  Description of Artificial Sequence: Synthetic peptide
RHHAVDAVVIALTGPR

SEQ ID NO: 129 - DNA, 99 bp, Artificial Sequence
  Description of Artificial Sequence: Synthetic oligonucleotide
ttcatagcatagccgtgtgagaaccgttgttatgataagaaatcttagaatttcgtaaagctctgcccctgtggccctcgtggttcaggggtatctttt

SEQ ID NO: 130 - DNA, 36 bp, Artificial Sequence
  Description of Artificial Sequence: Synthetic oligonucleotide
gtcatagcttccattctcacacggctatgctatgat

SEQ ID NO: 131 - protein, 19 aa, Artificial Sequence
  Description of Artificial Sequence: Synthetic peptide
VTYILGLDLGISSVGFAGI

SEQ ID NO: 132 - protein, 13 aa, Artificial Sequence
  Description of Artificial Sequence: Synthetic peptide
PDYIHIELSRDLG

SEQ ID NO: 133 - protein, 16 aa, Artificial Sequence
  Description of Artificial Sequence: Synthetic peptide
RHHAIDAIIVACTTEG

SEQ ID NO: 134 - RNA, 36 nt, Artificial Sequence
  Description of Artificial Sequence: Synthetic oligonucleotide
gucggagcagucgccggccaagugaucgaccgacac

SEQ ID NO: 135 - protein, 17 aa, Artificial Sequence
  Description of Artificial Sequence: Synthetic peptide
IACAVDLGLRNVGFATL

SEQ ID NO: 136 - protein, 14 aa, Artificial Sequence
  Description of Artificial Sequence: Synthetic peptide
ADAIVLEKLEGFIP

SEQ ID NO: 137 - protein, 12 aa, Artificial Sequence
  Description of Artificial Sequence: Synthetic peptide
RANSDHNASVNL

SEQ ID NO: 138 - protein, 57 aa, Artificial Sequence
  Description of Artificial Sequence: Synthetic polypeptide
CPFTGRAFGWTDVFGPSPTIDIEHIWPFSRSLDNSYLNKTLCDVNENRKIKRNQMPT

SEQ ID NO: 139 - protein, 53 aa, Artificial Sequence
  Description of Artificial Sequence: Synthetic polypeptide
SPYTGKPIPLSKLFTLEYEIEHIIPQSRMKNDSMSNLVISEAAVNDFKDRWLA

SEQ ID NO: 140 - protein, 57 aa, Artificial Sequence
  Description of Artificial Sequence: Synthetic polypeptide
CPYTGRGFGMGDLFGSNPTIDVEHILPFSRCLDNSFLNKTLCDVRENRLVKRNRTPF

SEQ ID NO: 141 - protein, 55 aa, Artificial Sequence
  Description of Artificial Sequence: Synthetic polypeptide
CPYSGSYIEPDEWASPTAVQIDHILPFSRSYDNSYMNKVLCTASANQEKGNKTPY

SEQ ID NO: 142 - protein, 7 aa, Artificial Sequence
  Description of Artificial Sequence: Synthetic peptide
  SITE (1)..(1): /note=“N terminus is linked to a peptide that contains a glutamic acid followed by a gap of unknown length”
  MOD_RES (4)..(6): Xaa can be any naturally occurring amino acid
ECNXXXH

SEQ ID NO: 143 - DNA, 12 bp, Artificial Sequence
  Description of Artificial Sequence: Synthetic oligonucleotide
atacagagtgcg

SEQ ID NO: 144 - DNA, 12 bp, Artificial Sequence
  Description of Artificial Sequence: Synthetic oligonucleotide
tatgtctcacgc

SEQ ID NO: 145 - DNA, 12 bp, Artificial Sequence
  Description of Artificial Sequence: Synthetic oligonucleotide
aaatttcccggg

SEQ ID NO: 146 - protein, 6 aa, Artificial Sequence
  Description of Artificial Sequence: Synthetic peptide
HHHHHH

SEQ ID NO: 147 - RNA, 36 nt, Artificial Sequence
  Description of Artificial Sequence: Synthetic oligonucleotide
guuuaagagaauaaagaaauuucuacuauuguagau

SEQ ID NO: 148 - DNA, 72 bp, Artificial Sequence
  Description of Artificial Sequence: Synthetic oligonucleotide
gagttcctaactctaagcgcccttgcgctttccccagccttcgggttggttgccttttagtgcaagggcgcg

SEQ ID NO: 149 - RNA, 72 nt, Artificial Sequence
  Description of Artificial Sequence: Synthetic oligonucleotide
gaguuccuaacucuaagcgcccuugcgcuuuccccagccuucggguugguugccuuuuagugcaagggcgcg

SEQ ID NO: 150 - RNA, 36 nt, Artificial Sequence
  Description of Artificial Sequence: Synthetic oligonucleotide
cucuagggcuaccccaaaauuucuacuauuguagau

SEQ ID NO: 151 - RNA, 36 nt, Artificial Sequence
  Description of Artificial Sequence: Synthetic oligonucleotide
cgcaauaagaccuauacaauuucuacuuuuguagau

SEQ ID NO: 152 - RNA, 64 nt, Artificial Sequence
  Description of Artificial Sequence: Synthetic oligonucleotide
cuccauaccggguuucccggcgcgugcgccgcaccgccgaucgccuuuccccgguuccucuugu

SEQ ID NO: 153 - RNA, 36 nt, Artificial Sequence
  Description of Artificial Sequence: Synthetic oligonucleotide
cucaaauaaaccuaucaaauuucuacuuucguagau

SEQ ID NO: 154 - RNA, 36 nt, Artificial Sequence
  Description of Artificial Sequence: Synthetic oligonucleotide
gcugucuuuaccuuucaaaacaggggcaguuacagc

SEQ ID NO: 155 - RNA, 36 nt, Artificial Sequence
  Description of Artificial Sequence: Synthetic oligonucleotide
guuguagcugcccugauuuugcaggguacacacaac

SEQ ID NO: 156 - RNA, 36 nt, Artificial Sequence
  Description of Artificial Sequence: Synthetic oligonucleotide
guuguugcuguucugauuuugcaggguagauacaac

SEQ ID NO: 157 - RNA, 36 nt, Artificial Sequence
  Description of Artificial Sequence: Synthetic oligonucleotide
cucgcagaccacgcccaaguggagggcgacugcacc

SEQ ID NO: 158 - RNA, 36 nt, Artificial Sequence
  Description of Artificial Sequence: Synthetic oligonucleotide
gcuguugaagccuuuauuuugaaagguaggugcagc

SEQ ID NO: 159 - RNA, 36 nt, Artificial Sequence
  Description of Artificial Sequence: Synthetic oligonucleotide
guuguuguagccucugauuugaaugguaggaacaac

SEQ ID NO: 160 - RNA, 36 nt, Artificial Sequence
  Description of Artificial Sequence: Synthetic oligonucleotide
guuguuuauacccuucaaaaaaagagcagugacaac

SEQ ID NO: 161 - RNA, 36 nt, Artificial Sequence
  Description of Artificial Sequence: Synthetic oligonucleotide
guuguuuuuaccccacaaaucaggagcaguuacaac

SEQ ID NO: 162 - RNA, 36 nt, Artificial Sequence
  Description of Artificial Sequence: Synthetic oligonucleotide
gccgugguuuggccggaauggucgcucugauacacu

SEQ ID NO: 163 - RNA, 35 nt, Artificial Sequence
  Description of Artificial Sequence: Synthetic oligonucleotide
aguguaucagagcgacuguuccgcuuaucacggua

SEQ ID NO: 164 - RNA, 66 nt, Artificial Sequence
  Description of Artificial Sequence: Synthetic oligonucleotide
aagggaacaaaaccgcgcagggcaaugucacagacaccccuucaacgccucccaguggggcguuuu

SEQ ID NO: 165 - RNA, 35 nt, Artificial Sequence
  Description of Artificial Sequence: Synthetic oligonucleotide
uuaucuuugaauuacgcuuuuaucaaaccauaaua

SEQ ID NO: 166 - RNA, 36 nt, Artificial Sequence
  Description of Artificial Sequence: Synthetic oligonucleotide
guugugguuugauuaaaagcguggauuaacgauauu

SEQ ID NO: 167 - RNA, 70 nt, Artificial Sequence
  Description of Artificial Sequence: Synthetic oligonucleotide
aaggauuauuccguagaaaacuaaucugcagccccauuucacgaaagugagaucgggguuguuguuuuuu

SEQ ID NO: 168 - RNA, 27 nt, Artificial Sequence
  Description of Artificial Sequence: Synthetic oligonucleotide
gugguuugauuaaaagcguggauuaac

SEQ ID NO: 169 - RNA, 36 nt, Artificial Sequence
  Description of Artificial Sequence: Synthetic oligonucleotide
aguguaucugagcgaugguugucgccuaucucagua

SEQ ID NO: 170 - RNA, 36 nt, Artificial Sequence
  Description of Artificial Sequence: Synthetic oligonucleotide
gcugggguucgucggcagccaucgcucagauacacu

SEQ ID NO: 171 - RNA, 36 nt, Artificial Sequence; identical to SEQ ID NO: 170
  Description of Artificial Sequence: Synthetic oligonucleotide

SEQ ID NO: 172 - RNA, 102 nt, Artificial Sequence
  Description of Artificial Sequence: Synthetic polynucleotide
aaaggacuuccauccgcagggccguacaucccgauucucccuccagcagggagagcacucuguacacccuucagggguggcuucuuagaagccgccccuuuu

SEQ ID NO: 173 - RNA, 37 nt, Artificial Sequence
  Description of Artificial Sequence: Synthetic oligonucleotide
uucauagcauagccgugugagaaccguuguuaugaua

SEQ ID NO: 174 - RNA, 36 nt, Artificial Sequence
  Description of Artificial Sequence: Synthetic oligonucleotide
gucauagcuuccauucucacacggcuaugcuaugau

SEQ ID NO: 175 - RNA, 36 nt, Artificial Sequence; identical to SEQ ID NO: 174
  Description of Artificial Sequence: Synthetic oligonucleotide

SEQ ID NO: 176 - RNA, 63 nt, Artificial Sequence
  Description of Artificial Sequence: Synthetic oligonucleotide
aagaaaucuuagaauuucguaaagcucugccccuguggcccucgugguucagggguaucuuuu

SEQ ID NO: 177 - RNA, 64 nt, Artificial Sequence
  Description of Artificial Sequence: Synthetic oligonucleotide
cuagguguauguuguuccgauguuaucgugagauacauuuuagccuucucaacauacaaauaau

SEQ ID NO: 178 - DNA, 100 bp, Artificial Sequence
  Description of Artificial Sequence: Synthetic polynucleotide
agaggcaacttgcagatttggtggcagctcaaaaattggctacaaaaccagttgatccaacagggcttgagcctgatgatcatctaaaggaaaaatcatc

SEQ ID NO: 179 - DNA, 78 bp, Artificial Sequence
  Description of Artificial Sequence: Synthetic oligonucleotide
  modified_base (72)..(78): n is a, c, g, or t
agaggcaacttgcagatttggtggcagctcaaaaataggctacaaaaccagttgatccaacagggcttgagnnnnnnn

SEQ ID NO: 180 - DNA, 55 bp, Artificial Sequence
  Description of Artificial Sequence: Synthetic oligonucleotide
  modified_base (35)..(55): n is a, c, g, or t
gatgatttttcctttagatgatcatcaggctcaannnnnnnnnnnnnnnnnnnnn

SEQ ID NO: 181 - DNA, 23 bp, Artificial Sequence
  Description of Artificial Sequence: Synthetic oligonucleotide
gatgatcatcaggctcaagccct

SEQ ID NO: 182 - RNA, 75 nt, Hantavirus
uauugauugacacggccauuaauuauauugagccugaugaucaucuaaagaauauaguuucauuuagaaaguaga

SEQ ID NO: 183 - RNA, 45 nt, Artificial Sequence
  Description of Artificial Sequence: Synthetic oligonucleotide
aaauuaaaacagggcuugagccugaugaucaucuaaaggaaaaau

SEQ ID NO: 184 - RNA, 45 nt, Artificial Sequence
  Description of Artificial Sequence: Synthetic oligonucleotide
aaaucccaacagggcuugagccugaugaucaucuaaagcggaaau

SEQ ID NO: 185 - RNA, 45 nt, Artificial Sequence
  Description of Artificial Sequence: Synthetic oligonucleotide
auuaauuaacagggcuugagccugaugaucaucuaaaguauaguu

SEQ ID NO: 186 - RNA, 45 nt, Artificial Sequence
  Description of Artificial Sequence: Synthetic oligonucleotide
aaauuaaaacagggcuugagccugaugaucaucuaaaguaaaaau

SEQ ID NO: 187 - RNA, 45 nt, Artificial Sequence
  Description of Artificial Sequence: Synthetic oligonucleotide
aaauuaaaacagggcuugagccugaugaucaucuaaagaaaaaau

SEQ ID NO: 188 - RNA, 45 nt, Artificial Sequence
  Description of Artificial Sequence: Synthetic oligonucleotide
aaauuaaaacagggcuugagccugaugaucaucuaaagauaaaau

SEQ ID NO: 189 - RNA, 45 nt, Artificial Sequence
  Description of Artificial Sequence: Synthetic oligonucleotide
aaauuaaaacagggcuugagccugaugaucaucuaaaguauaaau

SEQ ID NO: 190 - RNA, 45 nt, Artificial Sequence
  Description of Artificial Sequence: Synthetic oligonucleotide
aaauuaaaacagggcuugagccugaugaucaucuaaaggauaaau

SEQ ID NO: 191 - RNA, 45 nt, Artificial Sequence
  Description of Artificial Sequence: Synthetic oligonucleotide
aaauuaaaacagggcuugagccugaugaucaucuaaagaauaaau

SEQ ID NO: 192 - RNA, 45 nt, Artificial Sequence
  Description of Artificial Sequence: Synthetic oligonucleotide
aaauuauaacagggcuugagccugaugaucaucuaaaggauaaau

SEQ ID NO: 193 - RNA, 45 nt, Artificial Sequence
  Description of Artificial Sequence: Synthetic oligonucleotide
aaauuaaaacagggcuugagccugaugaucaucuaaaguuuaaau

SEQ ID NO: 194 - RNA, 45 nt, Artificial Sequence
  Description of Artificial Sequence: Synthetic oligonucleotide
aaauagaaacagggcuugagccugaugaucaucuaaaggauaaau

SEQ ID NO: 195 - RNA, 45 nt, Artificial Sequence
  Description of Artificial Sequence: Synthetic oligonucleotide
aaauacuaacagggcuugagccugaugaucaucuaaaggauaaau

SEQ ID NO: 196 - RNA, 45 nt, Artificial Sequence
  Description of Artificial Sequence: Synthetic oligonucleotide
aaauaagaacagggcuugagccugaugaucaucuaaaggauaaau

SEQ ID NO: 197 - protein, 6 aa, Artificial Sequence
  Description of Artificial Sequence: Synthetic peptide
  SITE (1)..(1): /note=“N terminus is linked to a peptide that contains a glutamic acid followed by a gap of unknown length”
RNKSFH

(19) United States
(12) Patent Application Publication
(10) Pub. No.: US 2022/0135958 A1
(43) Pub. Date: May 5, 2022

(54) CLASS 2 CRISPR-CAS RNA-GUIDED ENDONUCLEASES

(71) Applicant: CASPR BIOTECH CORPORATION, San Francisco, CA (US)

(72) Inventors: Carla Alejandra GIMÉNEZ, Buenos Aires (AR); Maria Julia LARA, Rosario (AR)

(21) Appl. No.: 17/541,405

(22) Filed: Dec. 3, 2021

Related U.S. Application Data
(63) Continuation of application No. PCT/US2021/057798, filed on Nov. 3, 2021.
(60) Provisional application No. 63/109,302, filed on Nov. 3, 2020.

(51) Int. Cl. C12N 9/22; C12N 15/11; C12N 9/12

(52) U.S. Cl. CPC C12N 9/22; C12N 2310/20; C12N 9/1276; C12N 15/11

(57) ABSTRACT

Provided herein are novel Class 2 Type II, Type V, type VI CRISPR-Cas RNA-guided endonucleases and systems comprising the same. Provided also are methods of making, and methods of use thereof. Exemplary methods of use include modifying target nucleic acids useful for therapeutic applications, and also include detecting targeting nucleic acids, useful for diagnostic applications.

The present application claims the benefit of U.S. Provisional Application No. 63/109,302 filed on Nov. 3, 2020, the entire contents of which are incorporated herein by reference.

### REFERENCE TO A SEQUENCE LISTING

This application contains a Sequence Listing in computer readable form. The computer readable form is incorporated herein by reference. Said ASCII copy, created on Nov. 2, 2021, is named 146401_091527_SL.txt and is 430,186 bytes in size.

### BACKGROUND

Prokaryotes have adaptive immune systems in place that utilize CRISPR (clustered regularly interspaced short palindromic repeats) and CRISPR-associated (Cas) proteins for RNA-guided nucleic acid cleavage to confer resistance to foreign genetic elements. The CRISPR-Cas systems act to confer adaptive immunity in bacteria and archaea via RNA-guided nucleic acid interference. To provide immunity against invaders, processed CRISPR array transcripts (crRNAs) assemble with Cas protein-containing surveillance complexes that recognize nucleic acids bearing sequence complementarity to the invader's derived segment of the crRNAs, known as the spacer.

Class 2 CRISPR-Cas systems are streamlined versions in which a single Cas protein (an effector endonuclease protein) bound to RNA is responsible for binding to and cleavage of a targeted sequence. The programmable nature of these minimal systems has facilitated their use as a versatile technology that continues to revolutionize the field of genome manipulation.

There however is a need for improved Class 2 CRISPR-Cas RNA-guided endonuclease variants. Provided herein are such variants, methods of making, methods of testing, and methods of using the same.

### SUMMARY

Provided herein are novel Class 2 Type II, Type V, and Type VI CRISPR-Cas RNA-guided proteins, methods of making, and methods of use. Also provided herein are engineered systems comprising the same.

In various embodiments, provided herein are compositions, pharmaceutical compositions, vectors, host cells, and kits comprising any of the proteins or polynucleotides of the engineered systems described herein.

Provided herein are novel Class 2 Type II, Type V, and Type VI CRISPR-Cas RNA-guided proteins, methods of making, and methods of use. Also provided herein are engineered systems comprising the same.

In various embodiments, provided herein are compositions, pharmaceutical compositions, vectors, host cells, and kits comprising any of the proteins or polynucleotides of the engineered systems described herein.

The disclosure relates to an engineered system that comprises a Class 2 CRISPR-Cas endonuclease or a nucleic acid encoding the endonuclease and a a gRNA or a nucleic acid encoding the gran. The Class 2 CRISPR-Cas endonuclease can be a Class 2 Type II CRISPR-Cas endonuclease comprising at least one of the RuvC sequences of Table 7, or a sequence comprising at least 60% sequence identity thereto. The Class 2 CRISPR-Cas endonuclease can be a Class 2 Type V CRISPR-Cas endonuclease comprising at least one of the RuvC sequences of Table 1, or a sequence comprising at least 60% sequence identity thereto. The Class 2 CRISPR-Cas endonuclease can be a Class 2 Type VI CRISPR-Cas endonuclease comprising at least one of the HEPN sequences of Table 4, or a sequence comprising at least 60% sequence identity thereto. The gRNA and the Class 2 CRISPR-Cas endonuclease generally do not naturally occur together. The gRNA can be capable of hybridizing to a target sequence in a target DNA or RNA. The gRNA can be capable of forming a complex with the Class 2 CRISPR-Cas endonuclease endonuclease.

The engineered system disclosed herein can comprise a Class 2 Type II CRISPR-Cas endonuclease; and a Class 2 Type II CRISPR-Cas gRNA. The gRNA can be a single-molecule gRNA. The gRNA can be a dual-molecule gRNA.

The endonuclease can be a Class 2 Type II CRISPR-Cas endonuclease comprising at least one of the RuvC or HNH sequences of Table 7, or a sequence comprising at least 60% sequence identity thereto or is a Class 2 Type V CRISPR-Cas endonuclease comprising at least one of the RuvC or HNH sequences of Table 1, or a sequence comprising at least 60% sequence identity thereto, and the target is target DNA.

The endonuclease is a Class 2 Type VI CRISPR-Cas endonuclease comprising at least one of the HEPN sequences of Table 4, or a sequence comprising at least 60% sequence identity thereto, and the target is target RNA.

The target RNA mRNA, tRNA, rRNA, miRNA, or siRNA.

The Class 2 Type II CRISPR-Cas endonuclease can comprise any one of SEQ ID NOS: 16-19, or a sequence comprising at least 60% sequence identity thereto. The Class 2 Type V CRISPR-Cas endonuclease can comprises any one of SEQ ID NOS: 1-7 or 20, or a sequence comprising at least 60% sequence identity thereto. The Class 2 Type VI CRISPR-Cas endonuclease can comprises any one of SEQ ID NOS: 8-15, or a sequence comprising at least 60% sequence identity thereto.

The disclosure relates to an engineered single-molecule gRNA that comprises a

targeter-RNA comprising a spacer sequence that is capable of hybridizing with a target sequence in a target DNA; and an activator-RNA that is capable of hybridizing with the targeter-RNA to form a double-stranded RNA duplex, the activator-RNA comprising a activator-RNA. The targeter-RNA and the activator-RNA can be covalently linked to one another. The single-molecule gRNA can be capable of forming a complex with a Class 2 Type II endonuclease. Hybridization of the spacer sequence to the target sequence can be capable of targeting the endonuclease to a target DNA. The Class 2 Type II CRISPR-Cas endonuclease can comprise at least one of the RuvC or HNH sequences of Table 7, or a sequence comprising at least 60% sequence identity thereto. The Class 2 Type II CRISPR-Cas endonuclease can comprise any one of SEQ ID NOS: 16-19, or a sequence comprising at least 60% sequence identity thereto. The targeter-RNA and the activator-RNA can be arranged in a 5′ to 3′ orientation. The activator-RNA and the targeter-RNA can be arranged in a 5′ to 3′ orientation. The targeter-RNA and the activator-RNA can be covalently linked to one another via a linker. The single-molecule gRNA can comprise one or more sequence modifications compared to a sequence of a corresponding wild type tracrRNA and/or crRNA. The targeter-RNA can comprise a spacer sequence of about 10-50 nucleotides that have 100% complementarity to a sequence in the target DNA. The targeter-RNA can comprise a spacer sequence of about 10-50 nucleotides that has less than 100% complementarity to a sequence in the target DNA.

Disclosed herein are methods of modifying a target DNA or RNA. The method can comprise contacting the target DNA with a CRISPR-Cas endonuclease system disclosed herein. The gRNA can hybridize with the target sequence, and modification of the target DNA or RNA occurs. The target can be RNA. The target can be mRNA, tRNA, rRNA, miRNA, or siRNA. The target can be DNA. The target DNA can be extrachromosomal DNA. The target DNA can be part of a chromosome. The target DNA can be part of a chromosome in vitro. The target DNA can be part of a chromosome in vivo. The target DNA or RNA can be outside a cell. The target DNA or RNA can be inside a cell. The target DNA or RNA can comprise a gene and/or its regulatory region.

The cell can be selected from the group consisting of: an archaeal cell, a bacterial cell, a eukaryotic cell, a eukaryotic single-cell organism, a somatic cell, a germ cell, a stem cell, a plant cell, an algal cell, an animal cell, in invertebrate cell, a vertebrate cell, a fish cell, a frog cell, a bird cell, a mammalian cell, a pig cell, a cow cell, a goat cell, a sheep cell, a rodent cell, a rat cell, a mouse cell, a non-human primate cell, and a human cell.

The modifying can comprise introducing a double strand break in a target DNA. The contacting can occur under conditions that are permissive for non-homologous end joining or homology-directed repair. The contacting can be with a target DNA to a donor polynucleotide. The donor polynucleotide, a portion of the donor polynucleotide, a copy of the donor polynucleotide, or a portion of a copy of the donor polynucleotide integrates into the target DNA. The method ma not comprise contacting the cell with a donor polynucleotide, or wherein the target DNA is modified such that nucleotides within the target DNA are deleted.

Disclosed herein are methods of detecting a target nucleic acid a sample, the method comprising contacting the sample with a Class 2 Type V CRISPR-Cas endonuclease comprising at least one of the RuvC sequences of Table 1, or a sequence comprising at least 60% sequence identity thereto; or a Class 2 Type VI CRISPR-Cas endonuclease comprising at least one of the HEPN sequences of Table 4, or a sequence comprising at least 60% sequence identity thereto, and a gRNA comprising a spacer sequence that is capable of hybridizing with a target sequence in a target nucleic acid; and a labeled detector that does not hybridize with the spacer sequence of the gRNA; and measuring a detectable signal produced by cleavage of the labeled detector by the endonuclease, thereby detecting the target nucleic acid. The Class 2 Type V CRISPR-Cas endonuclease can comprise any one of SEQ ID NOS: 1-7 or 20, or a sequence comprising at least 60% sequence identity thereto. The Class 2 Type VI CRISPR-Cas endonuclease comprises any one of SEQ ID NOS: 8-15, or a sequence comprising at least 60% sequence identity thereto. The labeled detector can comprise a labeled single stranded DNA. The labeled detector can comprise a labeled RNA. The labeled RNA can be a single stranded RNA. The labeled detector can comprise a labeled single stranded DNA/RNA chimera. The labeled detector can comprise one or more modified nucleotides. The target nucleic acid can be a single stranded DNA. The target nucleic acid can be double stranded DNA. The target nucleic acid can be single stranded RNA. The target nucleic acid can be viral, plant, fungal, or bacterial. The target sequence can be a sequence of a target provided in any of Tables 10a-10f. The target can be a coronvavirus. The target can be a SARS-CoV-2 virus. The target nucleic acid can be cDNA. The target nucleic acid can be from a human cell. The target nucleic acid can be from a human fetus or cancer cell. The sample can comprises cells. The sample can be urine, blood, serum, plasma, lymphatic fluid, cerebrospinal fluid, saliva, nasopharyngeal, oropharyngeal, nasopharyngeal/oropharyngeal, aspirate, or biopsy sample.

The methods disclosed herein can comprise determining an amount of the target nucleic acid present in the sample. Measuring a detectable signal can comprise one or more of: visual based detection, sensor based detection, color detection, gold nanoparticle based detection, fluorescence polarization, colloid phase transition/dispersion, electrochemical detection, and semiconductor-based sensing. The labeled detector can comprise a modified nucleobase, a modified sugar moiety, and/or a modified nucleic acid linkage. The detectable signal can be detectable in less than 15, 30, 45, 60, 90, 120, 150, 180, 210, or 240 minutes. The method can further comprise an amplification step selected from loop-mediated isothermal amplification (LAMP), helicase-dependent amplification (HDA), recombinase polymerase amplification (RPA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification (SPIA), signal mediated amplification of RNA technology (SMART), self-sustained sequence replication (3 SR), genome exponential amplification reaction (GEAR), and isothermal multiple displacement amplification (IMDA).

The target nucleic acid in the sample can be present at a concentration of less than 100 μM.

Disclosed herein are endonucleases comprising an amino acid sequence with 30%-99.5% homology to any one of SEQ ID NOs: 1-20.

Disclosed herein are compositions comprising a endonucleases described herein, and optionally a pharmaceutically acceptable carrier. The composition can comprise an endonucleases, optionally comprising a pharmaceutically acceptable carrier, a nucleic acid stabilizing buffer and/or or a endonuclease stabilizing buffer. The endonuclease can be lyopholized, and optionally further comprises any one or more of a labeled detector, a reverse transcriptase enzyme, and reagents for loop-mediated isothermal amplification.

The disclosure can comprise a recombinant expression vector comprising a DNA polynucleotide. The recombinant expression vector o can comprise nucleotide sequences encoding a single endonuclease that operably linked to a promoter.

A host cell comprising the DNA polynucleotide. A kit comprising one or more components of any of the engineered systems described herein. One or more components can be lyopholized. The one or more components can further comprise, a labeled reporter, and a gRNA directed to SARS-CoV-2.

### BRIEF DESCRIPTION OF THE DRAWINGS

FIG. 1 is a schematic representation of the organization of the CRISPR Cas loci around the Type V Cas_1 gene of the disclosure.

FIG. 2 shows the predicted secondary structure of the direct repeat for the Type V Cas_1 pre-crRNA. It is noted for this figure and all subsequent figures providing direct repeat (DR) sequences that while the sequence is provided in DNA nucleotides, it is understood that this DNA can then be transcribed into the pre-crRNA.

FIG. 3 shows the amino acid sequence of Type V Cas_1 (SEQ ID NO: 1) with the RuvC motifs underlined/highlighted.

FIG. 4 shows affinity purified Type V Cas_1's molecular weight and purity through SDS-PAGE. The arrow indicates the band containing the purified protein.

FIG. 5 shows a temperature-based assay to assess the stability of Type V Cas_1 protein.

FIGS. 6A-6B show ssDNA collateral cleavage of the Type V Cas_1 protein of the disclosures, complexed with a sgRNA for an exemplary Hantavirus target. The Type V Cas_1 exhibits collateral activity and can cut non-target containing ssDNA. FIG. 6A shows endpoint cleavage at 15, 20, 30 and 40 minutes; and FIG. 6B shows the time course of cleavage. (NTC): non-target control.

FIG. 7 shows activity of the Type V Cas_1 protein at different temperatures (25° C., 30° C., 38° C., and 50° C.).

FIG. 8 is a schematic representation of the organization of the CRISPR Cas loci around the Type V Cas_2 gene of the disclosure.

FIG. 9 shows the predicted secondary structure of an auxiliary RNA and its complementarity with the direct repeat (DR) for the Type V Cas_2 pre-crRNA. Complementary regions between the DR and the auxiliary RNA are indicated in bold. Base-complementarity between the DR and the auxiliary RNA is indicated by the lines.

FIG. 10 shows the amino acid sequence of Type V Cas_2 (SEQ ID NO: 2) with the RuvC motifs underlined/highlighted.

FIG. 11 shows affinity purified a Type V Cas_2's molecular weight and purity through SDS-PAGE.

FIG. 12 shows a temperature-based used to assay to assess the thermostability of the Type V Cas_2 protein.

FIG. 13 is a schematic representation of the organization of the CRISPR Cas cluster loci around the Type V Cas_3 gene of the disclosure.

FIG. 14 shows the predicted secondary structure of the direct repeat for the Type V Cas_3pre-crRNA.

FIG. 15 shows the amino acid sequence of Type V Cas_3 (SEQ ID NO: 3) with the RuvC motifs underlined/highlighted.

FIG. 16 is a schematic representation of the organization of the CRISPR Cas cluster loci around the Type V Cas_4 gene of the disclosure.

FIG. 17 shows the predicted secondary structure of the direct repeat for the Type V Cas_4 pre-crRNA.

FIG. 18 shows the amino acid sequence of Type V Cas_4 (SEQ ID NO: 4) with the RuvC motifs underlined/highlighted.

FIG. 19 is a schematic representation of the organization of the CRISPR Cas cluster loci around the Type V Cas_5 gene of the disclosure.

FIG. 20 shows the direct repeat sequence for the Type V Cas_5 pre-crRNA and the secondary structure of an auxiliary RNA for the Type V Cas_5. Base-complementarity between the direct repeat and the auxiliary RNA is indicated by the lines. Complementary regions between the DR and the auxiliary RNA are indicated in bold

FIG. 21 shows the amino acid sequence of Type V Cas_5 (SEQ ID NO: 5) with the RuvC motifs underlined/highlighted.

FIG. 22 is a schematic representation of the organization of the CRISPR Cas cluster loci around the Type V Cas_6 gene of the disclosure.

FIG. 23 shows the predicted secondary structure of an auxiliary RNA and its complementarity with the direct repeat for the pre-crRNA. Complementary regions between the DR and the auxiliary RNA are indicated in bold, and lines.

FIG. 24 shows the amino acid sequence of Type V Cas_6 (SEQ ID NO: 6) with the RuvC motifs underlined/highlighted.

FIG. 25 is a schematic representation of the organization of the CRISPR Cas cluster loci around the Type V Cas_7 gene of the disclosure.

FIG. 26 shows the predicted secondary structure of the direct repeat for the Type V Cas_7 pre-crRNA.

FIG. 27 shows the amino acid sequence of Type V Cas_7 (SEQ ID NO: 7) with the RuvC motifs underlined/highlighted.

FIG. 28 shows a Type V Cas_7's molecular weight and purity through SDS-PAGE.

FIG. 29 shows a temperature-based assay to assess the stability of the Type V Cas_7 protein.

FIG. 30 is a schematic representation of the organization of the CRISPR Cas cluster loci around the Type VI Cas_1 gene of the disclosure.

FIG. 31 shows the predicted secondary structure of the direct repeat for the Type VI Cas_1 pre-crRNA.

FIG. 32 shows the amino acid sequence of Type VI Cas_1 (SEQ ID NO: 8) with the HEPN motifs underlined/highlighted.

FIG. 33 is a schematic representation of the organization of the CRISPR Cas cluster loci around the Type VI Cas_2 gene of the disclosure.

FIG. 34 shows the predicted secondary structure of the direct repeat for the Type VI Cas_2 pre-crRNA.

FIG. 35 shows the amino acid sequence of Type VI Cas_2 (SEQ ID NO: 9) with the HEPN motifs underlined/highlighted.

FIG. 36 is a schematic representation of the organization of the CRISPR Cas cluster loci around the Type VI Cas_3 gene of the disclosure.

FIG. 37 shows the predicted secondary structure of the direct repeat for the Type VI Cas_3 pre-crRNA.

FIG. 38 shows the amino acid sequence of Type VI Cas_3 (SEQ ID NO: 10) with the HEPN motifs underlined/highlighted. The HEPN motifs (E . . . RxxxxH (SEQ ID NO: 93)) I and II are sequentially shown (highlighted in gray).

FIG. 39 is a schematic representation of the organization of the CRISPR Cas cluster loci around the Type VI Cas_4 gene of the disclosure.

FIG. 40 shows the predicted secondary structure of the direct repeat for the Type VI Cas_4 pre-crRNA.

FIG. 41 shows the amino acid sequence of Type VI Cas_4 (SEQ ID NO: 11) with the HEPN motifs underlined/highlighted.

FIG. 42 is a schematic representation of the organization of the CRISPR Cas cluster loci around the Type VI Cas_5 gene of the disclosure.

FIG. 43 shows the predicted secondary structure of the direct repeat for the Type VI Cas_5 pre-crRNA.

FIG. 44 shows the amino acid sequence of Type VI Cas_5 (SEQ ID NO: 12) with the HEPN motifs underlined/highlighted.

FIG. 45 is a schematic representation of the organization of the CRISPR Cas cluster loci around the Type VI Cas_6 gene of the disclosure.

FIG. 46 shows the predicted secondary structure of the direct repeat for the Type VI Cas_6 pre-crRNA.

FIG. 47 shows the amino acid sequence of Type VI Cas_6 (SEQ ID NO: 13). The HEPN motifs (E . . . RxxxxH (SEQ ID NO: 93)) I and II are sequentially shown (highlighted in gray).

FIG. 48 is a schematic representation of the organization of the CRISPR Cas cluster loci around the Type VI Cas_7 gene of the disclosure.

FIG. 49 shows the predicted secondary structure of the direct repeat for the Type VI Cas_7 pre-crRNA.

FIG. 50 shows the amino acid sequence of Type VI Cas_7 (SEQ ID NO: 14). The HEPN motifs (E . . . RxxxxH (SEQ ID NO: 93)) I and II are sequentially shown (highlighted in gray).

FIG. 51 is a schematic representation of the organization of the CRISPR Cas cluster loci around the Type VI Cas_8 gene of the disclosure.

FIG. 52 shows the predicted secondary structure of the direct repeat for the Type VI Cas_8 pre-crRNA.

FIG. 53 shows the amino acid sequence of Type VI Cas_8 (SEQ ID NO: 15). The HEPN motifs (E . . . RxxxxH (SEQ ID NO: 93)) I and II are sequentially shown (highlighted in gray).

FIG. 54 is a schematic representation of the organization of the CRISPR Cas cluster loci around the Type II Cas_1 gene of the disclosure.

FIG. 55 shows the sequence and the predicted secondary structure of the direct repeat and the tracrRNA (and their complementary regions for the Type II Cas_1.

FIG. 56 shows the amino acid sequence of Type II Cas_1 (SEQ ID NO: 16) with the RuvC motifs underlined/highlighted. The RuvC I, II and III motifs are sequentially shown (highlighted in gray). The conserved HNH domain is shown in italics. The Campylovacter_jeju Type II sequence referenced in Shmakov et al., 2015 was used as a reference for identification of the Ruv motifs.

FIG. 57 is a schematic representation of the organization of the CRISPR Cas cluster loci around the Type II Cas_2 gene of the disclosure.

FIG. 58 shows the sequence (upper part) and the predicted secondary structure (lower part) of the direct repeat and the tracrRNA, and their complementary regions for the Type II Cas_2.

FIG. 59 shows the amino acid sequence of Type II Cas_2 (SEQ ID NO: 17) with the RuvC motifs underlined/highlighted. The RuvC I, II and III motifs are sequentially shown (highlighted in gray). The conserved HNH domain is shown in italics. The Campylovacter_jeju Type II sequence referenced in Shmakov et al., 2015 was used as a reference for identification of the Ruv motifs.

FIG. 60 is a schematic representation of the organization of the CRISPR Cas cluster loci around the Type II Cas_3 gene of the disclosure.

FIG. 61 shows the sequence (lower part) and the predicted secondary structure (upper part) of the direct repeat and the tracrRNA, and their complementary regions for the Type II Cas_3.

FIG. 62 shows the amino acid sequence of Type II Cas_3 (SEQ ID NO: 18) with the RuvC motifs underlined/highlighted. The RuvC I, II and III motifs are sequentially shown (highlighted in gray). The conserved HNH domain is shown in italics. The Campylovacter_jeju Type II sequence referenced in Shmakov et al., 2015 was used as a reference for identification of the Ruv motifs.

FIG. 63 is a schematic representation of the organization of the CRISPR Cas cluster loci around the Type II Cas_4 gene of the disclosure.

FIG. 64 shows the sequence (lower part) and the predicted secondary structure (upper part) of the direct repeat and the tracrRNA (top right), and their complementary regions (top left) for the Type II Cas_4.

FIG. 65 shows the amino acid sequence of Type II Cas_4 (SEQ ID NO: 19) with the RuvC motifs underlined/highlighted. The RuvC I, II and III motifs are sequentially shown (highlighted in gray). The conserved HNH domain is shown in italics.

FIG. 66 is a schematic representation of the organization of the CRISPR Cas cluster loci around the Type V Cas_8 gene of the disclosure.

FIG. 67 shows the predicted secondary structure of the direct repeat for the Type V Cas_8 pre-crRNA.

FIG. 68 shows the amino acid sequence of Type V Cas_8 (SEQ ID NO: 20) with the RuvC motifs underlined/highlighted.

FIGS. 69A-69B are graphs showing colateral activity for Type V Cas_1protein complexes using substrate single stranded DNA (FIG. 69A) and dsDNA (FIG. 69B) as target in the presence of magnesium or manganese as an additive. FIG. 69A shows time course cleavage using a single stranded DNA target. FIG. 69B shows time course cleavage using a double stranded DNA target.

FIGS. 70A-70B are graphs showing trans-cleavage activities of Type V Cas_1protein on single-strand DNA (FIG. 70A) and hybrid reporters but not on the single-stranded RNA tested (FIG. 70B).

FIG. 71 shows specific double strand DNA cleavage site of the Type V Cas_1 protein.

FIG. 72 shows trans-cleavage activities of the Type V Cas_2protein using MnCl₂ as additive at defined temperature range.

FIG. 73 shows the activity of Type V Cas_2 protein in a temperature curve (32.8° C.-45° C.).

FIG. 74 shows a graph depicting differential efficiency in dinucleotide reporter cleavage.

FIG. 75 shows affinity purified a Type V Cas_3's molecular weight and purity through SDS-PAGE.

FIG. 76 shows a graph of a temperature-based assay to assess the stability of Type V Cas_3 protein.

FIGS. 77A-77D shows graphs of a Type V Cas_3. Activity test in different reaction buffer conditions.

FIG. 78 is a graph showing activity of the Type V Cas_3 protein at a gradient temperature, from 30° C. to 50° C.

FIGS. 79A-79B are graphs showing DNA reporter cleavage (FIG. 79A) and RNA reporter cleavage (FIG. 79B) for Type V Cas_3.

FIG. 80 shows affinity purified Type V Cas_4's molecular weight and purity through SDS-PAGE. The arrow indicates the band containing the purified protein.

FIG. 81 shows a temperature-based assay to assess the stability of Type V Cas_4 protein.

FIGS. 82A-82C shows Type V Cas_4 trans-cleavage activity in three different commercial buffers, a curve of pH and different salt concentrations.

FIG. 83 shows the activity of Type V Cas_4 protein at different temperatures (30° C.-50° C.).

FIGS. 84A-84B are graphs showing DNA reporter cleavage (FIG. 84A) and RNA reporter cleavage (FIG. 84B) for Type V Cas_4.

FIG. 85 shows affinity purified Type V Cas_5's molecular weight and purity through SDS-PAGE.

FIG. 86 shows a melt curve for Type V Cas_5, Type V Cas_5 with RNA guide, and protein buffer (C-).

FIG. 87 shows a graph of the activity test in different buffer conditions. Shows ssDNA collateral cleavage of the Type V Cas_5 protein complexed with a scoutRNA and a sgRNA of two different lengths (18 and 24 nucleotides) for an exemplary ssDNA Hantavirus target. Three buffer conditions were tested for each sgRNA.

FIG. 88 Shows trans-cleavage activities of the Type V Cas_5 protein in different buffer conditions at a defined temperature range.

FIGS. 89A-89B shows double stranded DNA (FIG. 89A) and single stranded DNA (FIG. 89B) PAM selection for Type V Cas_21_1.

FIG. 90 shows Type V Cas_5 trans-cleavage activity in dinucleotide single-stranded DNA reporters.

FIG. 91 Shows Type V Cas_5 trans-cleavage activity single-base polynucleotides single-stranded DNA reporters.

FIGS. 92A-92B shows ssRNA trans-cleavage activity in different buffer solutions of the Type VI Cas_2 protein complexed with a sgRNA for an exemplary ssRNA Hantavirus target. FIG. 92A shows time course cleavage over 3 h. FIG. 92B shows the endpoint activity after 180 min.

FIGS. 93A-93B shows ssRNA trans-cleavage activity of the Type VI Cas_2 protein at a defined temperature range. FIG. 93A shows time course cleavage over 3 h. FIG. 93B shows the endpoint activity after 180 min.

FIG. 94 shows affinity purified Type VI Cas_2's molecular weight and purity through SDS-PAGE. The arrow indicates the band containing the purified protein.

FIGS. 95A-95B shows ssRNA trans-cleavage activity of the Type VI Cas_2 protein complexed with a sgRNA for an exemplary ssRNA Hantavirus target with variable flanking sequences at its 5′ and 3′ ends.

FIG. 96 shows the percentage of trans-cleavage activity for different ssRNA reporters of the Type VI Cas_2 protein complexed with a sgRNA for an exemplary ssRNA Hantavirus target.

FIGS. 97A-97B are graphs showing ssRNA and ssDNA trans-cleavage activity of the Type VI Cas_2 protein complexed with a sgRNA for an exemplary ssRNA or ssDNA Hantavirus target. FIG. 97A shows time course cleavage using ssRNA target; and FIG. 97B shows the time course cleavage using ssDNA target. Type VI Cas_Psm protein was used as control.

FIG. 98 shows ssRNA trans-cleavage activity in different buffer solutions of the Type VI Cas_4 protein complexed with a sgRNA for an exemplary ssRNA Hantavirus target.

FIG. 99 shows the trans-cleavage preference for different ssRNA reporters of the Type VI Cas_4 protein complexed with a sgRNA for an exemplary ssRNA Hantavirus target.

FIG. 100 shows affinity purified Type VI Cas_4's molecular weight and purity through SDS-PAGE.

FIG. 101 shows ssRNA trans-cleavage activity of the Type VI Cas_4 protein at a defined temperature range.

FIG. 102 shows ssRNA and ssDNA trans-cleavage activity of the Type VI Cas_4 protein complexed with a sgRNA for an exemplary ssRNA or ssDNA Hantavirus target.

### DETAILED DESCRIPTION

Provided herein are novel Class 2 Type II, V, and VI CRISPR-Cas RNA-guided endonucleases, systems, methods of making, and methods of use.

### Definitions

The terms “polynucleotide” and “nucleic acid,” used interchangeably herein, refer to a polymeric form of nucleotides of any length, either ribonucleotides or deoxyribonucleotides. Thus, terms “polynucleotide” and “nucleic acid” encompass single-stranded DNA; double-stranded DNA; multi-stranded DNA; single-stranded RNA; double-stranded RNA; multi-stranded RNA; genomic DNA; cDNA; DNA-RNA hybrids; and a polymer comprising purine and pyrimidine bases or other natural, chemically or biochemically modified, non-natural, or derivatized nucleotide bases.

By “hybridizable” or “complementary” or “substantially complementary” it is meant that a nucleic acid (e.g. RNA, DNA) comprises a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength.

It is understood that a sequence of a polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure, a ‘bulge’, and the like).

Percent complementarity and determination of percent identity or homology between particular stretches of nucleic acid sequences or within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489). Other programs, algorithms, and methods are available to the skilled artisan and may be utilized.

Determination of percent identity between particular stretches of polypeptides can be determined using any convenient method. Seeral programs, algorithms, and methods are available to the skilled artisan and may be utilized.

Methods of determining sequence similarity or identity between two or more nucleic acid or amino acid sequences are known in the art. Sequence similarity or identity may be determined for an entire length of a nucleic acid or amino acid, or for an indicated portion thereof. Sequence similarity or identity may be determined using standard techniques, including, but not limited to, the local sequence identity algorithm of Smith & Waterman, Adv. Appl. Math. 2, 482 (1981), by the sequence identity alignment algorithm of Needleman & Wunsch, J Mol. Biol. 48,443 (1970), by the search for similarity method of Pearson & Lipman, Proc. Natl. Acad. Sci. USA 85, 2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12, 387-395 (1984), or by inspection. Another suitable algorithm is the BLAST algorithm, described in Altschul et al., J Mol. Biol. 215, 403-410, (1990) and Karlin et al., Proc. Natl. Acad. Sci. USA 90, 5873-5787 (1993). Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAST, Novoalign (Novocraft Technologies; available at www.novocraft.com), ELAND (Illumina, San Diego, Calif.), SOAP (available at soap.genomics.org.cn), and Maq (available at maq.sourceforge.net). An exemplary useful BLAST program is the WU-BLAST-2 program which was obtained from Altschul et al., Methods in Enzymology, 266, 460-480 (1996); http://blast.wustl/edu/blast/README.html. WU-BLAST-2 uses several search parameters, which are optionally set to the default values. The parameters are dynamic values and are established by the program itself depending upon the composition of the particular sequence and composition of the particular database against which the sequence of interest is being searched; however, the values may be adjusted to increase sensitivity. Further, an additional useful algorithm is gapped BLAST as reported by Altschul et al, (1997) Nucleic Acids Res. 25, 3389-3402.

The terms “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

A “vector” or “expression vector” is a replicon, such as plasmid, phage, virus, or cosmid, to which another DNA segment, i.e. an “insert”, may be attached so as to bring about the replication of the attached segment in a cell.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise DNA or RNA.

In general, a guide sequence is any polynucleotide sequence having sufficient complementarity with a target polynucleotide sequence to hybridize with the target sequence and direct sequence-specific binding of a CRISPR complex to the target sequence. The term “targeting sequence” means the portion of a guide sequence having sufficient complemenarity with a target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a Type V endonuclease” includes a plurality of such endonucleases and reference to “the gRNA” or “the guide RNA” includes reference to one or more gRNAs and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Class 2 CRISPR-Cas systems generally have single-polypeptide multidomain nuclease effectors, and comprises Types II, V, and VI.

Class 2 Type II CRISPR-Cas endonucleases are RNA-guided DNA endonucleases (interchangeably referred to herein as Type II endonucleases, Type II endonucleases and the like). Exemplary Type II endonucleases include Cas9.

Class 2 Type V CRISPR-Cas endonucleases are RNA-guided DNA endonucleases (interchangeably referred to herein as Type V endonucleases, Type V endonucleases and the like), and further possess collateral activity. Exemplary Type V endonucleases include Cas12 (inclusive of all subtypes) and Cas14 (inclusive of all subtypes).

Class 2 Type VI CRISPR-Cas endonucleases are RNA-guided RNA endonucleases (interchangeably referred to herein as Type VI endonucleases, Type VI endonucleases and the like), and further possess collateral activity. Exemplary Type VI endonucleases include Cas13 (inclusive of all subtypes). Type VI endonucleases achieve RNA cleavage through conserved basic residues within its two HEPN domains. The target RNA, i.e. the RNA of interest, is the RNA to be targeted leading to the recruitment to, and the binding of the Type VI endonuclease at, the target site of interest on the target RNA.

Accordingly provided herein are novel Type II, Type V, and Type VI CRISPR-Cas RNA-guided endonucleases.

### I. Class 2 Type V CRISPR-Cas RNA-Guided Systems

Provided herein are novel Class 2 Type V CRISPR-Cas RNA-guided endonucleases and their gRNAs, constituting the novel Class 2 Type V CRISPR-Cas RNA-guided systems of the disclosure.

Provided herein are engineered systems comprising: a Class 2 Type V CRISPR-Cas RNA-guided endonuclease of the disclosure and a single guide RNA, wherein the gRNA and the Class 2 Type V CRISPR-Cas RNA-guided endonuclease do not naturally occur together, wherein the gRNA is capable of hybridizing to a target sequence in a target DNA, wherein the gRNA is capable of forming a complex with the Class 2 Type V CRISPR-Cas RNA-guided endonuclease, and wherein the Class 2 Type V CRISPR-Cas RNA-guided endonuclease possesses collateral activity and is capable of collaterally cleaving a single stranded polynucleotide comprising RNA, without the use of a tracrRNA.

The components of the system described in turn below.

### Type V CRISPR-Cas RNA-Guided Endonucleases

Provided herein are novel Type V CRISPR-Cas RNA-guided endonucleases. In some embodiments, these endonucleases may share certain structural, sequence, and/or functional similarities with any one of the subtypes of Cas12. In some embodiments, these endonucleases may share certain structural, sequence, and/or functional similarities with any one of the subtypes of Cas14.

Type V endonucleases of the are capable of cleaving target single stranded DNA (e.g. Cas14-like Type V endonucleases) and target double stranded DNA (e.g. Cas12-like Type V endonucleases). Type V endonucleases additionally possess collateral activity.

Without being bound to any theory or mechanism, a Type V CRISPR-Cas RNA-guided endonucleases of the disclosure comprise three RuvC motifs, responsible for catalytic activity.

In some embodiments a Type V CRISPR-Cas RNA-guided endonuclease of the disclosure comprises any one of the RuvC sequences of Table 1, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type V CRISPR-Cas RNA-guided endonuclease of the disclosure comprises any two of the RuvC sequences of Table 1, or sequences comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type V CRISPR-Cas RNA-guided endonuclease of the disclosure comprises any three of the RuvC sequences of Table 1, or sequences comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type V CRISPR-Cas RNA-guided endonuclease of the disclosure comprises a RuvC I motif selected from the group consisting of SEQ ID NO: 62, SEQ ID NO: 67, SEQ ID NO: 71, SEQ ID NO: 75, SEQ ID NO: 80, SEQ ID NO: 85, SEQ ID NO: 89, and SEQ ID NO: 135, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type V CRISPR-Cas RNA-guided endonuclease of the disclosure comprises a RuvC II motif selected from the group consisting of SEQ ID NO: 63, SEQ ID NO: 68, SEQ ID NO: 72, SEQ ID NO: 76, SEQ ID NO: 81, SEQ ID NO: 86, SEQ ID NO: 90, and SEQ ID NO: 136, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type V CRISPR-Cas RNA-guided endonuclease of the disclosure comprises a RuvC III motif selected from the group consisting of SEQ ID NO: 64, SEQ ID NO: 69, SEQ ID NO: 73, SEQ ID NO: 77, SEQ ID NO: 82, SEQ ID NO: 87, SEQ ID NO: 91, and SEQ ID NO: 137, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type V CRISPR-Cas RNA-guided endonuclease of the disclosure comprises a (1) RuvC I motif selected from the group consisting of SEQ ID NO: 62, SEQ ID NO: 67, SEQ ID NO: 71, SEQ ID NO: 75, SEQ ID NO: 80, SEQ ID NO: 85, SEQ ID NO: 89, and SEQ ID NO: 135, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; (2) a RuvC II motif selected from the group consisting of SEQ ID NO: 63, SEQ ID NO: 68, SEQ ID NO: 72, SEQ ID NO: 76, SEQ ID NO: 81, SEQ ID NO: 86, SEQ ID NO: 90, and SEQ ID NO: 136, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; and (3) a RuvC III motif selected from the group consisting of SEQ ID NO: 64, SEQ ID NO: 69, SEQ ID NO: 73, SEQ ID NO: 77, SEQ ID NO: 82, SEQ ID NO: 87, SEQ ID NO: 91, and SEQ ID NO: 137, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type V CRISPR-Cas RNA-guided endonuclease of the disclosure comprises a (1) RuvC I motif comprising the sequence of SEQ ID NO: 62, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; (2) a RuvC II motif comprising the sequence of SEQ ID NO: 63, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; and (3) a RuvC III motif comprising the sequence of SEQ ID NO: 64, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type V CRISPR-Cas RNA-guided endonuclease of the disclosure comprises a (1) RuvC I motif comprising the sequence of SEQ ID NO: 67, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; (2) a RuvC II motif comprising the sequence of SEQ ID NO: 68, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; and (3) a RuvC III motif comprising the sequence of SEQ ID NO: 69, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type V CRISPR-Cas RNA-guided endonuclease of the disclosure comprises a (1) RuvC I motif comprising the sequence of SEQ ID NO: 71, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; (2) a RuvC II motif comprising the sequence of SEQ ID NO: 72, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; and (3) a RuvC III motif comprising the sequence of SEQ ID NO: 73, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type V CRISPR-Cas RNA-guided endonuclease of the disclosure comprises a (1) RuvC I motif comprising the sequence of SEQ ID NO: 75, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; (2) a RuvC II motif comprising the sequence of SEQ ID NO: 76, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; and (3) a RuvC III motif comprising the sequence of SEQ ID NO: 77, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type V CRISPR-Cas RNA-guided endonuclease of the disclosure comprises a (1) RuvC I motif comprising the sequence of SEQ ID NO: 80, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; (2) a RuvC II motif comprising the sequence of SEQ ID NO: 81, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; and (3) a RuvC III motif comprising the sequence of SEQ ID NO: 82, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type V CRISPR-Cas RNA-guided endonuclease of the disclosure comprises a (1) RuvC I motif comprising the sequence of SEQ ID NO: 85, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; (2) a RuvC II motif comprising the sequence of SEQ ID NO: 86, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; and (3) a RuvC III motif comprising the sequence of SEQ ID NO: 87, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type V CRISPR-Cas RNA-guided endonuclease of the disclosure comprises a (1) RuvC I motif comprising the sequence of SEQ ID NO: 89, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; (2) a RuvC II motif comprising the sequence of SEQ ID NO: 90, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; and (3) a RuvC III motif comprising the sequence of SEQ ID NO: 91, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type V CRISPR-Cas RNA-guided endonuclease of the disclosure comprises a (1) RuvC I motif comprising the sequence of SEQ ID NO: 135, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; (2) a RuvC II motif comprising the sequence of SEQ ID NO: 136, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; and (3) a RuvC III motif comprising the sequence of SEQ ID NO: 137, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

Table 1 provided exemplary RuvC I, RuvC II, RuvC III sequences of the Type V endonucleases of the disclosure.

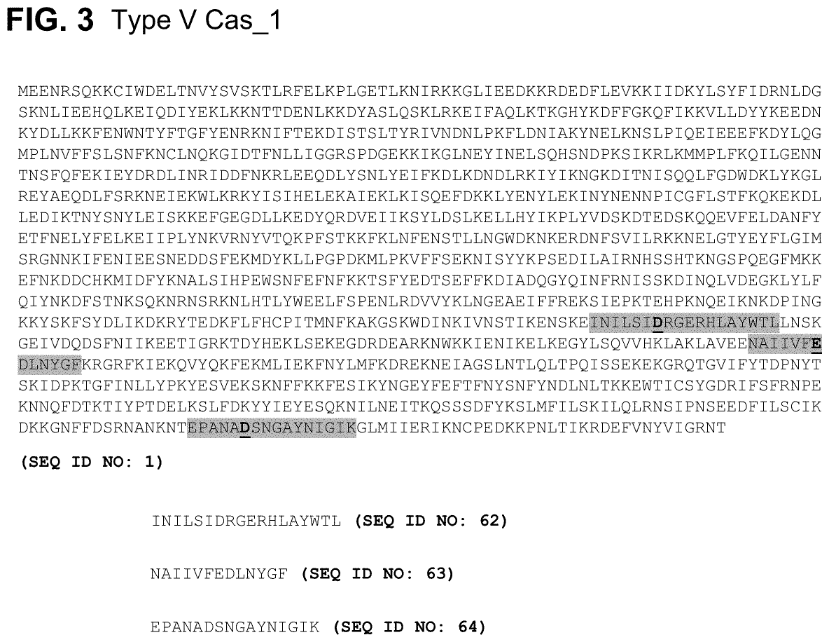

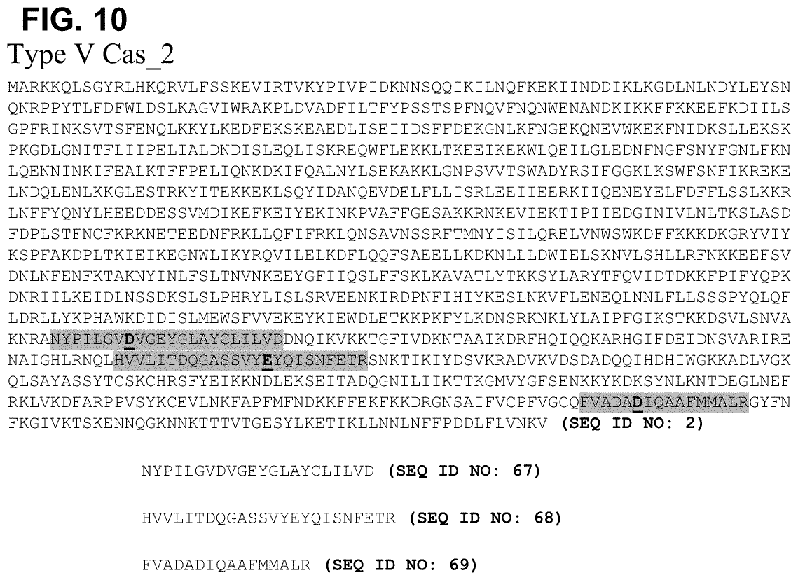

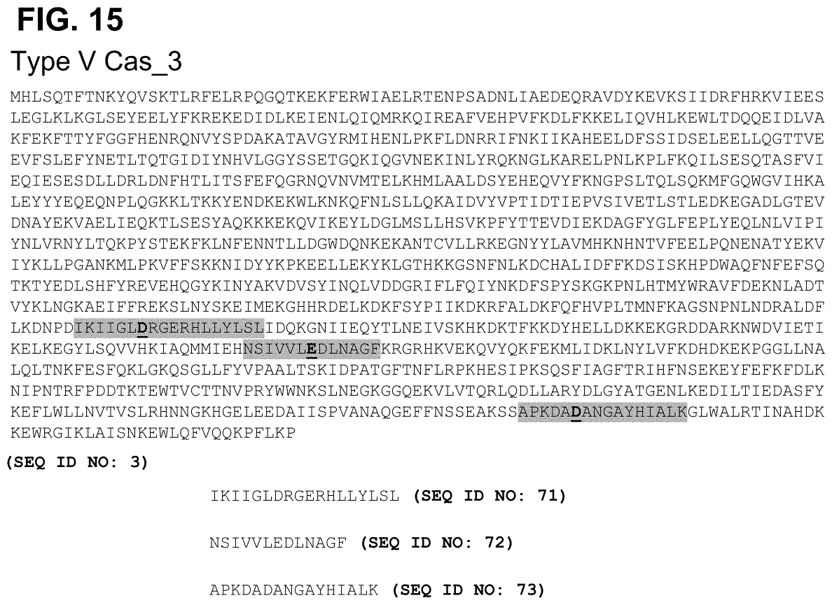

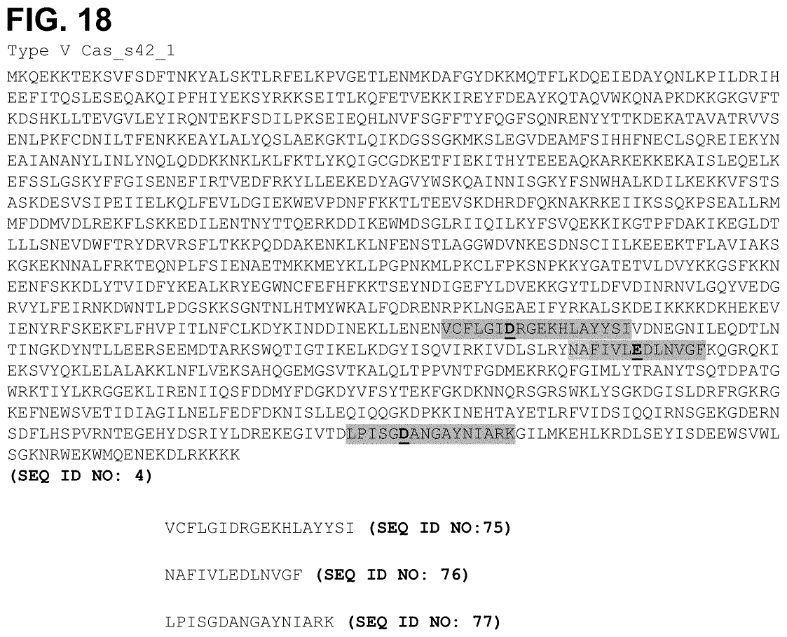

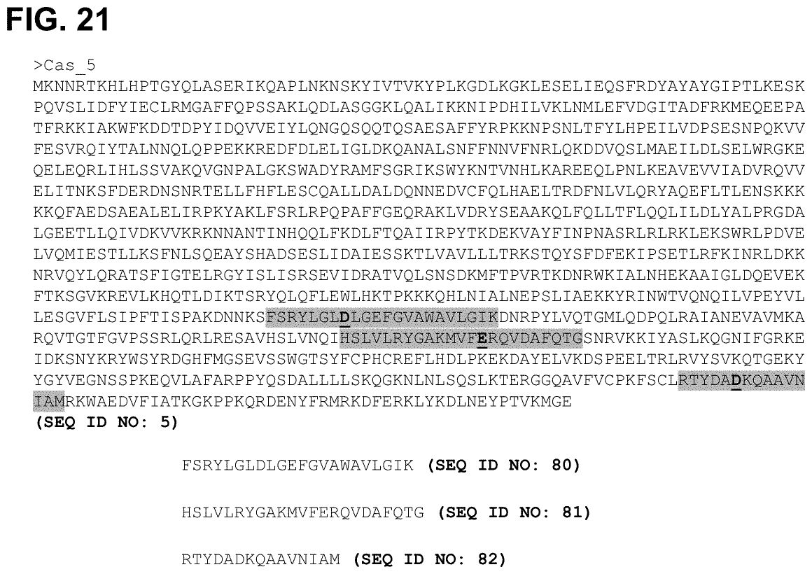

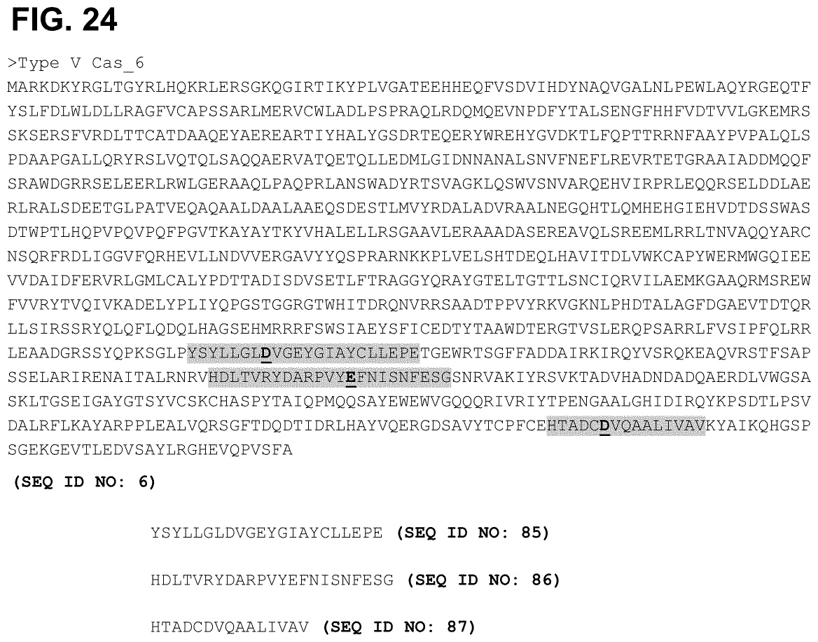

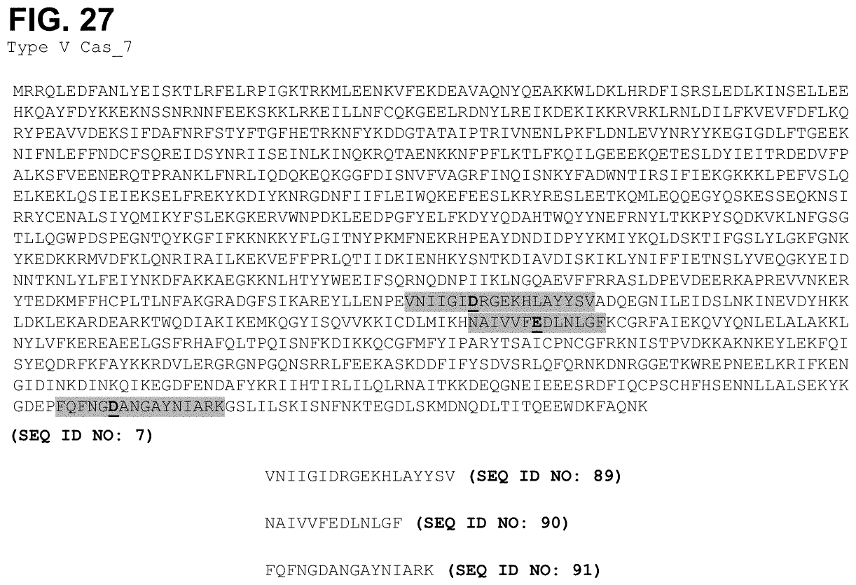

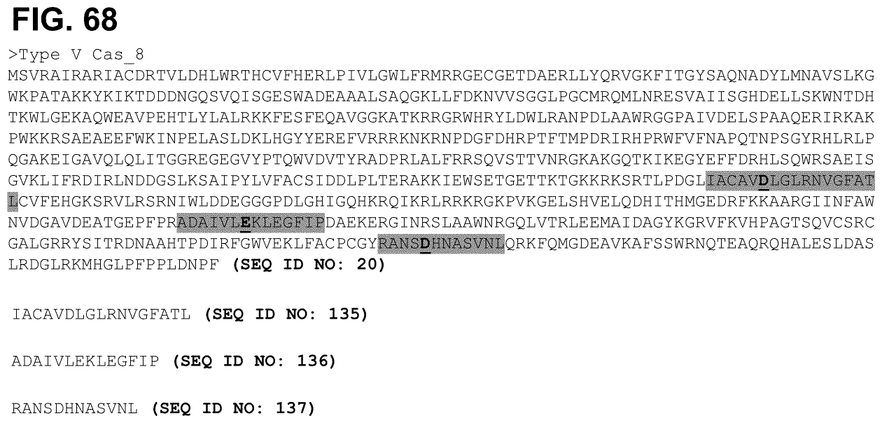

TABLE 1

| SEQ | | | |
|---|---|---|---|
| ID | Exemplary | | |
| NO: | Figure | MOTIF | SEQUENCE |
| 62 | FIG. 3 | RuvC I | INILSIDRGERHLAYWTL |
| 63 | FIG. 3 | RuvC II | NAIIVFEDLNYGF |
| 64 | FIG. 3 | RuvC III | EPANADSNGAYNIGIK |
| 67 | FIG. 10 | RuvC I | NYPILGVDVGEYGLAYCLI |
| | | | LVD |
| 68 | FIG. 10 | RuvC II | HVVLITDQGASSVYEYQIS |
| | | | NFETR |
| 69 | FIG. 10 | RuvC III | FVADADIQAAFMMALR |
| 71 | FIG. 15 | RuvC I | IKIIGLDRGERHLLYLSL |
| 72 | FIG. 15 | RuvC II | NSIVVLEDLNAGF |
| 73 | FIG. 15 | RuvC III | APKDADANGAYHIALK |
| 75 | FIG. 18 | RuvC I | VCFLGIDRGEKHLAYYSI |
| 76 | FIG. 18 | RuvC II | NAFIVLEDLNVGF |
| 77 | FIG. 18 | RuvC III | LPISGDANGAYNIARK |
| 80 | FIG. 21 | RuvC I | FSRYLGLDLGEFGVAWAVL |
| | | | GIK |
| 81 | FIG. 21 | RuvC II | HSLVLRYGAKMVFERQVDA |
| | | | FQTG |
| 82 | FIG. 21 | RuvC III | RTYDADKQAAVNIAM |
| 85 | FIG. 24 | RuvC I | YSYLLGLDVGEYGIAYCLL |
| | | | EPE |
| 86 | FIG. 24 | RuvC II | HDLTVRYDARPVYEFNISN |
| | | | FESG |
| 87 | FIG. 24 | RuvC III | HTADCDVQAALIVAV |
| 89 | FIG. 27 | RuvC I | VNIIGIDRGEKHLAYYSV |
| 90 | FIG. 27 | RuvC II | NAIVVFEDLNLGF |
| 91 | FIG. 27 | RuvC III | FQFNGDANGAYNIARK |
| 135 | FIG. 68 | RuvC I | IACAVDLGLRNVGFATL |
| 136 | FIG. 68 | RuvC II | ADAIVLEKLEGFIP |
| 137 | FIG.68 | RuvC III | RANSDHNASVNL |

Table 2 provides exemplary amino acid sequences for certain Type V sequences of the disclosure. Genes were identified from metagenomic samples. Scripts were run on the sequences, designed to find CRISPR sequences and accompanying genes encoding proteins showing homology with reported Cas enzymes. Comparative BlastP analyses were performed against sequences deposited in databases (NCBI, LENS), discarding those candidates showing Id % >50 with deposited proteins. Presence of specific domains (e.g. RuvC, HEPN) and catalytic motifs were determined (CD-search, phmmer, UNIPROT).

TABLE 2

| | FIGURE | |
|---|---|---|
| NAME | SEQ ID NO. | AMINO ACID SEQUENCE |
| TYPE V CAS_1 | FIG. 3 | MEENRSQKKCIWDELTNVYSVSKTLRFELKPLGETLKNIRKKGLIEEDKKR |
| | SEQ ID NO: | DEDFLEVKKIIDKYLSYFIDRNLDGSKNLIEEHQLKEIQDIYEKLKKNTTDEN |
| | 1 | LKKDYASLQSKLRKEIFAQLKTKGHYKDFFGKQFIKKVLLDYYKEEDNKY |
| | | DLLKKFENWNTYFTGFYENRKNIFTEKDISTSLTYRIVNDNLPKFLDNIAKY |
| | | NELKNSLPIQEIEEEFKDYLQGMPLNVFFSLSNFKNCLNQKGIDTFNLLIGGR |
| | | SPDGEKKIKGLNEYINELSQHSNDPKSIKRLKMMPLFKQILGENNTNSFQFE |
| | | KIEYDRDLINRIDDFNKRLEEQDLYSNLYEIFKDLKDNDLRKIYEKNGKDITN |
| | | ISQQLFGDWDKLYKGLREYAEQDLFSRKNEIEKWLKRKYISIHELEKAIEKL |
| | | KISQEFDKKLYENYLEKINYNENNPICGFLSTFKQKEKDLLEDIKTNYSNYL |
| | | EISKKEFGEGDLLKEDYQRDVEIIKSYLDSLKELLHYIKPLYVDSKDTEDSK |
| | | QQEVFELDANEYETENELYFELKEIIPLYNKVRNYVTQKPFSTKKFKLNFEN |
| | | STLLNGWDKNKERDNFSVILRKKNELGTYEYFLGEMSRGNNKIFENIEESNE |
| | | DDSFEKMDYKLLPGPDKMLPKVFFSEKNISYYKPSEDILAIRNHSSHTKNGS |
| | | PQEGFMKKEENKDDCHKMEDFYKNALSHIPEWSNFEENFKKTSFYEDTSEF |
| | | FKDIADQGYQINFRNISSKDINQLVDEGKLYLFQIYNKDFSTNKSQKNRNSR |
| | | KNLHTLYWEELFSPENLRDVVYKLNGEAEIFFREKSIEPKTEHPKNQEIKNK |
| | | DPINGKKYSKFSYDLEKDKRYTEDKFLFHCPITMNFKAKGSKWDINKIVNST |
| | | EKENSKEINILSEDRGERHLAYWTLLNSKGEIVDQDSFNIIKEETIGRKTDYHE |
| | | KLSEKEGDRDEARKNWKKIENIKELKEGYLSQVVHKLAKLAVEENAIIVFE |
| | | DLNYGEKRGRFKIEKQVYQKFEKMLIEKFNYLMFKDREKNEIAGSLNTLQL |
| | | TPQISSEKEKGRQTGVIFYTDPNYTSKIDPKTGFINLLYPKYESVEKSKNFFK |
| | | KFESIKYNGEYFEFTFNYSNFYNDLNLTKKEWTICSYGDRIFSFRNPEKNNQ |
| | | FDTKTIYPTDELKSLFDKYYIEYESQKNILNEITKQSSSDFYKSLMFILSKILQ |
| | | LRNSIPNSEEDFILSCIKDKKGNFFDSRNANKNTEPANADSNGAYNIGIKGL |
| | | MIIERIKNCPEDKKPNLTIKRDEFVNYVIGRNT |
| Type V Cas_2 | FIG. 10 | MARKKQLSGYRLHKQRVLFSSKEVIRTVKYPIVPIDKNNSQQIKILNQFKEK |
| | SEQ ID NO: | IINDDIKLKGDLNLNDYLEYSNQNRPPYTLFDFWLDSLKAGVIWRAKPLDV |
| | 2 | ADFILTFYPSSTSPFNQVFNQNWENANDKIKKFFKKEEFKDIILSGPFRINKS |
| | | VTSFENQLKKYLKEDFEKSKEAEDLISEIEDSFFDEKGNLKFNGEKQNEVWK |
| | | EKFNIDKSLLEKSKPKGDLGNITFLIIPELIALDNDISLEQLISKREQWFLEKK |
| | | LTKEEIKEKWLQEILGLEDNFNGFSNYFGNLFKNLQENNINKIFEALKTFFPE |
| | | LIQNKDKIFQALNYLSEKAKKLGNPSVVTSWADYRSIFGGKLKSWFSNFIK |
| | | REKELNDQLENLKKGLESTRKYITEKKEKLSQYIDANQEVDELFLLISRLEEI |
| | | IEERKIIQENEYELFDFFLSSLKKRLNFFYQNYLHEEDDESSVMDIKEFKEIYE |
| | | KINKPVAFFGESAKKRNKEVIEKTIPIIEDGINIVLNLTKSLASDFDPLSTFNC |
| | | FKRKNETEEDNFRKLLQFIFRKLQNSAVNSSRFTMNYISILQRELVNWSWK |
| | | DFFKKKDKGRYVIYKSPFAKDPLTKIEIKEGNWLIKYRQVILELKDFLQQFS |
| | | AEELLKDKNLLLDWIELSKNVLSHLLRENKKEEFSVDNLNFENEKTAKNYI |
| | | NLFSLTNVNIKEEYGFIIQSLFFSKLKAVATLYTKKSYLARYTFQVIDTDKKF |
| | | PIFYQPKDNRIILKEIDLNSSDKSLSLPHRYLISLSRVEENKIRDPNFIHIYKES |
| | | LNKVFLENEQLNNLFLLSSSPYQLQFLDRLLYKPHAWKDEDISLMEWSFVV |
| | | EKEYKIEWDLETKKPKFYLKDNSRKNKLYLAIPFGEKSTKKDSVLSNVAKN |
| | | RANYPILGVDVGEYGLAYCLILVDDNQIKVKKTGFIVDKNTAAIKDRFHQI |
| | | QQKARHGIFDEIDNSVARIRENAIGHLRNQLHVVLITDQGASSVYEYQISNF |
| | | ETRSNKTIKIYDSVKRADVKVDSDADQQIHDHIWGKKADLVGKQLSAYAS |
| | | SYTCSKCHRSFYEIKKNDLEKSEITADQGNILIEKTTKGMVYGFSENKKYKD |
| | | KSYNLKNTDEGLNEFRKLVKDFARPPVSYKCEVLNKFAPFMFNDKKFFEK |
| | | FKKDRGNSAIFVCPFVGCQFVADADIQAAFMMALRGYENFKGIVKTSKEN |
| | | NQGKNNKTTTVTGESYLKETIKLLNNLNFFPDDLFLVNKV |
| Type V Cas_3 | FIG. 15 | MHLSQTFTNKYQVSKTLRFELRPQGQTKEKFERWIAELRTENPSADNLIAE |
| | SEQ ID NO: | DEQRAVDYKEVKSIEDRFHRKVIEESLEGLKLKGLSEYEELYFKREKEDIDL |
| | 3 | KEIENLQIQMRKQIREAFVEHPVFKDLFKKELIQVHLKEWLTDQQEIDLVAK |
| | | FEKFTTYFGGFHENRQNVYSPDAKATAVGYRMIHENLPKFLDNRRIFNKIIK |
| | | AHEELDFSSIDSELEELLQGTTVEEVFSLEFYNETLTQTGEDIYNHVLGGYSS |
| | | ETGQKIQGVNEKINLYRQKNGLKARELPNLKPLFKQILSESQTASEVIEQIES |
| | | ESDLLDRLDNFHTLITSFEFQGRNQVNVMTELKHMLAALDSYEHEQVYFK |
| | | NGPSLTQLSQKMFGQWGVIHKALEYYYEQEQNPLQGKKLTKKYENDKEK |
| | | WLKNKQFNLSLLQKAEDVYVPTIDTIEPVSIVETLSTLEDKEGADLGTEVDN |
| | | AYEKVAELIEQKTLSESYAQKKKEKQVIKEYLDGLMSLLHSVKPFYTTEVD |
| | | IEKDAGFYGLFEPLYEQLNLVIPIYNLVRNYLTQKPYSTEKFKLNFENNTLL |
| | | DGWDQNKEKANTCVLLRKEGNYYLAVMHKNHNTVFEELPQNENATYEK |
| | | VIYKLLPGANKMLPKVFFSKKNEDYYKPKEELLEKYKLGTHKKGSNFNLKD |
| | | CHALIDFFKDSISKIIPDWAQFNFEFSQTKTYEDLSHFYREVEHQGYKINYA |
| | | KVDVSYINQLVDDGRIFLFQIYNKDFSPYSKGKPNLHTMYWRAVFDEKNL |
| | | ADTVYKLNGKAEIFFREKSLNYSKEIMEKGHHRDELKDKFSYPIIKDKRFAL |
| | | DKFQFHVPLTMNFKAGSNPNLNDRALDFLKDNPDIKIIGLDRGERHLLYLS |
| | | LIDQKGNIIEQYTLNEIVSKHKDKTFKKDYHELLDKKEKGRDDARKNWDVI |
| | | ETIKELKEGYLSQVVHKIAQMMIEHNSIVVLEDLNAGFKRGRHKVEKQVY |
| | | QKFEKMLIDKLNYLVFKDHDKEKPGGLLNALQLTNKFESFQKLGKQSGLL |
| | | FYVPAALTSKIDPATGETNFLRPKHESIPKSQSFIAGETRIHENSEKEYFEFKE |
| | | DLKNIPNTRFPDDTKTEWTVCTTNVPRYWWNKSLNEGKGGQEKVLVTQR |
| | | LQDLLARYDLGYATGENLKEDILTIEDASFYKEFLWLLNVTVSLRHNNGKH |
| | | GELEEDAIISPVANAQGEFFNSSEAKSSAPKDADANGAYHIALKGLWALRTI |
| | | NAHDKKEWRGIKLAISNKEWLQFVQQKPFLKP |
| TYPE V CAS_4 | FIG. 18 | MKQEKKTEKSVFSDFTNKYALSKTLRFELKPVGETLENMKDAFGYDKKM |
| | SEQ ID NO: | QTFLKDQEIEDAYQNLKPILDRIHEEFITQSLESEQAKQIPFHIYEKSYRKKSE |
| | 4 | ITLKQFETVEKKIREYFDEAYKQTAQVWKQNAPKDKKGKGVFTKDSHKLL |
| | | TEVGVLEYIRQNTEKFSDILPKSEIEQHLNVFSGFFTYFQGFSQNRENYYTTK |
| | | DEKATAVATRVVSENLPKFCDNILTFENKKEAYLALYQSLAEKGKTLQIKD |
| | | GSSGKMKSLEGVDEAMFSIHHFNECLSQREIEKYNEAIANANYLINLYNQL |
| | | QDDKKNKLKLFKTLYKQIGCGDKETFIEKITHYTEEEAQKARKEKKEKAIS |
| | | LEQELKEFSSLGSKYFFGISENEFIRTVEDFRKYLLEEKEDYAGVYWSKQAI |
| | | NNISGKYFSNWHALKDILKEKKVFSTSASKDESVSIPEIIELKQLFEVLDGIE |
| | | KWEVPDNFFKKTLTEEVSKDHRDFQKNAKRKEIIKSSQKPSEALLRMMFD |
| | | DMVDLREKFLSKKEDILENTNYTTQERKDDIKEWMDSGLRIIQILKYFSVQE |
| | | KKIKGTPFDAKEKEGLDTLLLSNEVDWFTRYDRVRSFLTKKPQDDAKENKL |
| | | KLNFENSTLAGGWDVNKESDNSCIILKEEEKTFLAVIAKSKGKEKNNALFR |
| | | KTEQNPLFSIENAETMKKMEYKLLPGPNKMLPKCLFPKSNPKKYGATETVL |
| | | DVYKKGSFKKNEENFSKKDLYTVIDFYKEALKRYEGWNCFEFHFKKTSEY |
| | | NDIGEFYLDVEKKGYTLDFVDINRNVLGQYVEDGRVYLFEIRNKDWNTLP |
| | | DGSKKSGNTNLHTMYWKALFQDRENRPKLNGEAEIFYRKALSKDEIKKKK |
| | | DKHEKEVIENYRFSKEKFLFHVPITLNFCLKDYKINDDINEKLLENENVCFL |
| | | GIDRGEKHLAYYSIVDNEGNILEQDTLNTINGKDYNTLLEERSEEMDTARK |
| | | SWQTIGTEKELKDGYISQVIRKIVDLSLRYNAFIVLEDLNVGFKQGRQKIEKS |
| | | VYQKLELALAKKLNFLVEKSAHQGEMGSVTKALQLTPPVNTFGDMEKRK |
| | | QFGIMLYTRANYTSQTDPATGWRKTIYLKRGGEKLIRENIIQSFDDMYFDG |
| | | KDYVFSYTEKFGKDKNNQRSGRSWKLYSGKDGISLDRFRGKRGKEFNEWS |
| | | VETIDIAGILNELFEDFDKNISLLEQIQQGKDPKKINEHTAYETLRFVIDSIQQI |
| | | RNSGEKGDERNSDFLHSPVRNTEGEHYDSRIYLDREKEGIVTDLPISGDANG |
| | | AYNIARKGILMKEHLKRDLSEYISDEEWSVWLSGKNRWEKWMQENEKDL |
| | | RKKKK |
| Type V Cas_5 | FIG. 15 | MKNNRTKHLHPTGYQLASERIKQAPLNKNSKYIVTVKYPLKGDLKGKLES |
| | SEQ ID NO: | ELIEQSFRDYAYAYGIPTLKESKPQVSLIDFYIECLRMGAFFQPSSAKLQDLA |
| | 5 | SGGKLQALIKKNIPDHILVKLNMLEFVDGITADFRKMEQEEPATFRKKIAK |
| | | WFKDDTDPYIDQVVEIYLQNGQSQQTQSAESAFFYRPKKNPSNLTFYLHPEI |
| | | LVDPSESNPQKVVFESVRQIYTALNNQLQPPEKKREDFDLELIGLDKQANA |
| | | LSNFFNNVFNRLQKDDVQSLMAEILDLSELWRGKEQELEQRLIHLSSVAKQ |
| | | VGNPALGKSWADYRAMFSGRIKSWYKNTVNHLKAREEQLPNLKEAVEVV |
| | | IADVRQVVELITNKSFDERDNSNRTELLFHFLESCQALLDALDQNNEDVCF |
| | | QLHAELTRDFNLVLQRYAQEFLTLENSKKKKKQFAEDSAEALELIRPKYAK |
| | | LFSRLRPQPAFFGEQRAKLVDRYSEAAKQLFQLLTFLQQLILDLYALPRGD |
| | | ALGEETLLQIVDKVVKRKNNANTINHQQLFKDLFTQAIIRPYTKDEKVAYFI |
| | | NPNASRLRLRKLEKSWRLPDVELVQMIESTLLKSFNLSQEAYSHADSESLID |
| | | AIESSKTLVAVLLLTRKSTQYSFDFEKIPSETLRFKINRLDKKNRVQYLQRA |
| | | TSFIGTELRGYISLISRSEVEDRATVQLSNSDKMFTPVRTKDNRWKIALNHEK |
| | | AAIGLDQEVEKFTKSGVKREVLKHQTLDIKTSRYQLQFLEWLHKTPKKKQ |
| | | HLNIALNEPSLIAEKKYRINWTVQNQILVPEYVLLESGVFLSIPFTISPAKDN |
| | | NKSFSRYLGLDLGEFGVAWAVLGIKDNRPYLVQTGMLQDPQLRAIANEVA |
| | | VMKARQVTGTFGVPSSRLQRLRESAVHSLVNQUISLVLRYGAKMVFERQV |
| | | DAFQTGSNRVKKIYASLKQGNIFGRKEIDKSNYKRYWSYRDGHFMGSEVS |
| | | SWGTSYFCPHCREFLHDLPKEKDAYELVKDSPEELTRLRVYSVKQTGEKY |
| | | YGYVEGNSSPKEQVLAFARPPYQSDALLLLSKQGKNLNLSQSLKTERGGQ |
| | | AVFVCPKFSCLRTYDADKQAAVNIAMRKWAEDVFIATKGKPPKQRDENYF |
| | | RMRKDFERKLYKDLNEYPTVKMGE |
| Type V Cas_6 | FIG. 24 | MARKDKYRGLTGYRLHQKRLERSGKQGIRTIKYPLVGATEEHHEQFVSDVI |
| | SEQ ID NO: | HDYNAQVGALNLPEWLAQYRGEQTFYSLFDLWLDLLRAGFVCAPSSARL |
| | 6 | MERVCWLADLPSPRAQLRDQMQEVNPDFYTALSENGFHHFVDTVVLGKE |
| | | MRSSKSERSFVRDLTTCATDAAQEYAEREARTIYHALYGSDRTEQERYWR |
| | | EHYGVDKTLFQPTTRRNFAAYPVPALQLSPDAAPGALLQRYRSLVQTQLSA |
| | | QQAERVATQETQLLEDMLGIDNNANALSNVFNEFLREVRTETGRAAIADD |
| | | MQQFSRAWDGRRSELEERLRWLGERAAQLPAQPRLANSWADYRTSVAGK |
| | | LQSWVSNVARQEHVIRPRLEQQRSELDDLAERLRALSDEETGLPATVEQAQ |
| | | AALDAALAAEQSDESTLMVYRDALADVRAALNEGQHTLQMHEHGIEHVD |
| | | TDSSWASDTWPTLHQPVPQVPQFPGVTKAYAYTKYVHALELLRSGAAVLE |
| | | RAAADASEREAVQLSREEMLRRLTNVAQQYARCNSQRFRDLIGGVFQRHE |
| | | VLLNDVVERGAVYYQSPRARNKKPLVELSHTDEQLHAVITDLVWKCAPY |
| | | WERMWGQIEEVVDAIDFERVRLGMLCALYPDTTADISDVSETLFTRAGGY |
| | | QRAYGTELTGTTLSNCIQRVILAEMKGAAQRMSREWFVVRYTVQIVKADE |
| | | LYPLIYQPGSTGGRGTWHITDRQNVRRSAADTPPVYRKVGKNLPHDTALA |
| | | GFDGAEVTDTQRLLSIRSSRYQLQFLQDQLHAGSEHMRRRFSWSIAEYSFIC |
| | | EDTYTAAWDTERGTVSLERQPSARRLFVSIPFQLRRLEAADGRSSYQPKSG |
| | | LPYSYLLGLDVGEYGIAYCLLEPETGEWRTSGFFADDAIRKIRQYVSRQKE |
| | | AQVRSTFSAPSSELARIRENAITALRNRVHDLTVRYDARPVYEFNISNFESGS |
| | | NRVAKIYRSVKTADVHADNDADQAERDLVWGSASKLTGSEIGAYGTSYV |
| | | CSKCHASPYTAIQPMQQSAYEWEWVGQQQRIVRIYTPENGAALGHIDIRQY |
| | | KPSDTLPSVDALRFLKAYARPPLEALVQRSGFTDQDTIDRLHAYVQERGDS |
| | | AVYTCPFCEHTADCDVQAALIVAVKYAIKQHGSPSGEKGEVTLEDVSAYL |
| | | RGHEVQPVSFA |
| Type V Cas_7 | FIG. 27 | MRRQLEDFANLYEISKTLRFELRPIGKTRKMLEENKVFEKDEAVAQNYQEA |
| | SEQ ID NO: | KKWLDKLHRDFISRSLEDLKINSELLEEHKQAYFDYKKEKNSSNRNNFEEK |
| | 7 | SKKLRKEILLNFCQKGEELRDNYLREEKDEKIKKRVRKLRNLDILFKVEVFD |
| | | FLKQRYPEAVVDEKSIFDAFNRFSTYFTGFHETRKNFYKDDGTATAIPTRIV |
| | | NENLPKFLDNLEVYNRYYKEGIGDLFTGEEKNIFNLEFFNDCFSQREEDSYN |
| | | RIISEINLKINQKRQTAENKKNFPFLKTLFKQILGEEEKQETESLDYIEITRDE |
| | | DVFPALKSFVEENERQTPRANKLFNRLIQDQKEQKGGFDISNVFVAGRFINQ |
| | | ISNKYFADWNTIRSIFIEKGKKKLPEFVSLQELKEKLQSIEIEKSELFREKYKD |
| | | IYKNRGDNFIIFLEIWQKEFEESLKRYRESLEETKQMLEQQEGYQSKESSEQ |
| | | KNSIRRYCENALSIYQMIKYFSLEKGKERVWNPDKLEEDPGFYELFKDYYQ |
| | | DAHTWQYYNEFRNYLTKKPYSQDKVKLNFGSGTLLQGWPDSPEGNTQYK |
| | | GFIFKKNKKYFLGITNYPKMFNEKRHPEAYDNDIDPYYKMIYKQLDSKTIF |
| | | GSLYLGKFGNKYKEDKKRMVDFKLQNRIRAILKEKVEFFPRLQTIIDKIENH |
| | | KYSNTKDIAVDISKIKLYNIFFIETNSLYVEQGKYEIDNNTKNLYLFEIYNKD |
| | | FAKKAEGKKNLHTYYWEEIFSQRNQDNPIIKLNGQAEVFFRRASLDPEVDE |
| | | ERKAPREVVNKERYTEDKMFFHCPLTLNFAKGRADGFSIKAREYLLENPEV |
| | | NIIGIDRGEKHLAYYSVADQEGNILEIDSLNKINEVDYHKKLDKLEKARDEA |
| | | RKTWQDIAKIKEMKQGYISQVVKKICDLMIKHNAIVVIAEDLNLGFKCGRFA |
| | | IEKQVYQNLELALAKKLNYLVFKEREAEELGSFRHAFQLTPQISNFKDIKKQ |
| | | CGFMFYIPARYTSAICPNCGFRKNISTPVDKKAKNKEYLEKFQISYEQDRFK |
| | | FAYKKRDVLERGRGNPGQNSRRLFEEKASKDDFIFYSDVSRLQFQRNKDN |
| | | RGGETKWREPNEELKRIFKENGIDINKDINKQIKEGDFENDAFYKRIIHTIRLI |
| | | LQLRNAITKKDEQGNEIEEESRDFIQCPSCHFHSENNLLALSEKYKGDEPFQ |
| | | FNGDANGAYNIARKGSLILSKISNFNKTEGDLSKMDNQDLTITQEEWDKFA |
| | | QNK |
| Type V Cas_8 | FIG. 68 | MSVRAIRARIACDRTVLDHLWRTHCVFHERLPIVLGWLFRMRRGECGETD |
| | SEQ ID NO: | AERLLYQRVGKFITGYSAQNADYLMNAVSLKGWKPATAKKYKIKTDDDN |
| | 20 | GQSVQISGESWADEAAALSAQGKLLFDKNVVSGGLPGCMRQMLNRESVAI |
| | | ISGHDELLSKWNTDHTKWLGEKAQWEAVPEHTLYLALRKKFESFEQAVGG |
| | | KATKRRGRWHRYLDWLRANPDLAAWRGGPAIVDELSPAAQERIRKAKPW |
| | | KKRSAEAEEFWKINPELASLDKLHGYYEREFVRRRKNKRNPDGFDHRPTFT |
| | | MPDRIRHPRWFVFNAPQTNPSGYRHLRLPQGAKEIGAVQLQLITGGREGEG |
| | | VYPTQWVDVTYRADPRLALFRRSQVSTTVNRGKAKGQTKIKEGYEFFDRH |
| | | LSQWRSAEISGVKLIFRDIRLNDDGSLKSAIPYLVFACSIDDLPLTERAKKIE |
| | | WSETGETTKTGKKRKSRTLPDGLIACAVDLGLRNVGFATLCVFEHGKSRVL |
| | | RSRNIWLDDEGGGPDLGHIGQHKRQIKRLRRKRGKPVKGELSHVELQDHIT |
| | | HMGEDRFKKAARGIINFAWNVDGAVDEATGEPFPRADAIVLEKLEGFIPDA |
| | | EKERGINRSLAAWNRGQLVTRLEEMAIDAGYKGRVFKVHPAGTSQVCSRC |
| | | GALGRRYSITRDNAAHTPDIRFGWVEKLFACPCGYRANSDHNASVNLQRK |
| | | FQMGDEAVKAFSSWRNQTEAQRQHALESLDASLRDGLRKMEIGLPFPPLD |
| | | NPF |

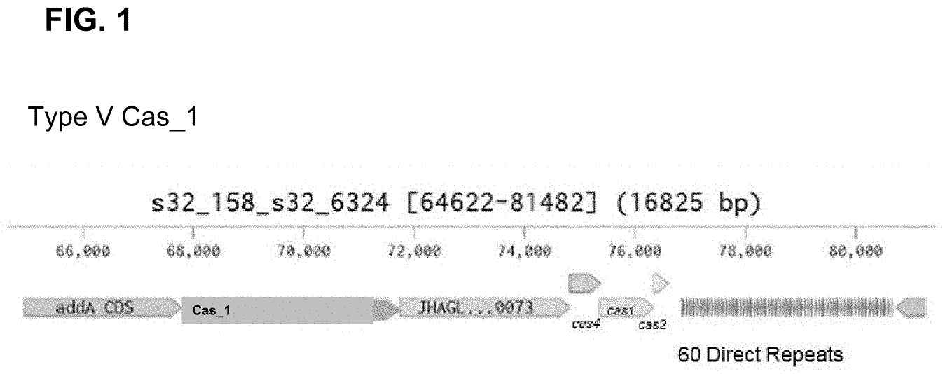

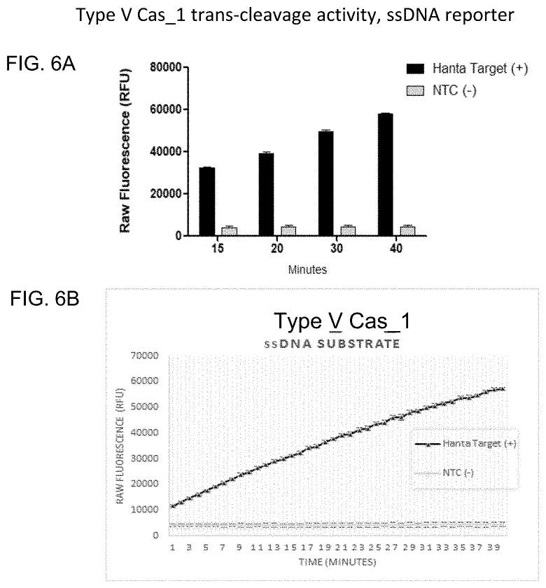

SEQ ID NO: 1 represents a novel Type V variant of the disclosure, Type V Cas_1, (1283 amino acids in length). FIG. 1 is a schematic representation of the organization of the CRISPR Cas loci around the Type V Cas_1gene of the disclosure. The loci has 60 direct repeats. FIG. 3 shows the amino acid sequence of Type V Cas_1 (SEQ ID NO: 1) with the RuvC motifs underlined/highlighted. The FnCas12a sequence referenced in Shmakov et al., 2015 was used as a reference for identification of the Ruv motifs. The RuvC I, II and III motifs are sequentially shown (highlighted in gray, with the conserved catalytic amino acids underlined). FIG. 6 shows that Type V Cas_1 exhibits trans-cleavage activity on single-stranded DNA reporter. It is noted that

In some embodiments the Type V CRISPR-Cas RNA-guided endonuclease of the disclosure comprises the amino acid sequence of SEQ ID NO: 1 and proteins with at least 30%-99.5% sequence identity thereto. Accordingly, provided herein are proteins comprising the amino acid sequence of SEQ ID NO: 1 and proteins with at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto. Also provided herein are nucleic acids encoding the proteins comprising the amino acid sequence of SEQ ID NO: 1 and proteins with at least 30%-99.5% sequence identity thereto.

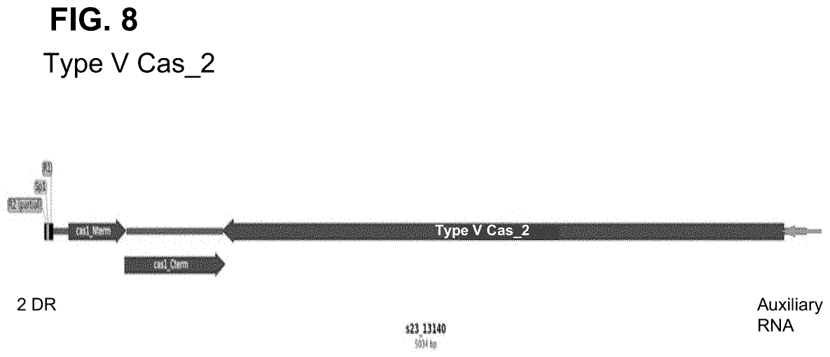

SEQ ID NO: 2 represents a novel Type V variant of the disclosure, Type V Cas_2, (1235 amino acids in length). FIG. 8 is a schematic representation of the organization of the CRISPR Cas loci around the Type V Cas_2 gene of the disclosure. It is noted that the organization is similar to the casY genetic organization (referencing Chen et al. 2018, 10.3389/fmicb.2019.00928), but not identical (for example, the cas1 gene is split into separate open reading frames). The loci has 2 direct repeats.

In some embodiments the Type V CRISPR-Cas RNA-guided endonuclease of the disclosure comprises the amino acid sequence of SEQ ID NO: 2 and proteins with at least 30%-99.5% sequence identity thereto. Accordingly, provided herein are proteins comprising the amino acid sequence of SEQ ID NO: 2 and proteins with at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto. Also provided herein are nucleic acids encoding the proteins comprising the amino acid sequence of SEQ ID NO: 2 and proteins with at least 30%-99.5% sequence identity thereto.

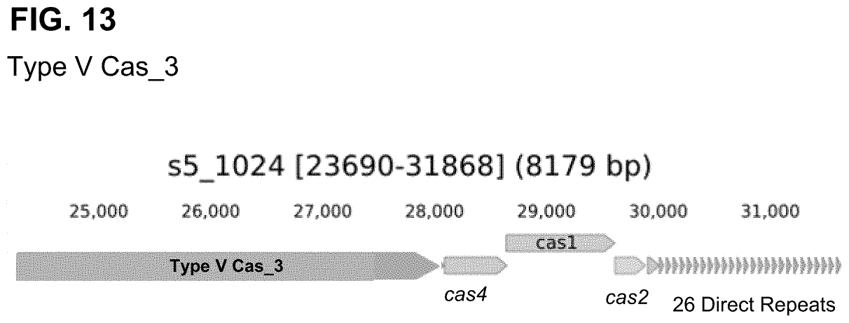

SEQ ID NO: 3 represents a novel Type V variant of the disclosure, Type V Cas_3, (1259 amino acids in length). FIG. 13 is a schematic representation of the organization of the CRISPR Cas cluster loci around the novel Type V Cas_3 gene of the disclosure. FIG. 15 shows the amino acid sequence of Type V Cas_3 (SEQ ID NO: 3) with the RuvC motifs underlined/highlighted. The FnCas12a sequence referenced in Shmakov et al., 2015 was used as a reference for identification of the Ruv motifs. The RuvC I, II and III motifs are sequentially shown (highlighted in gray, with the conserved catalytic amino acids underlined)

In some embodiments the Type V CRISPR-Cas RNA-guided endonuclease of the disclosure comprises the amino acid sequence of SEQ ID NO: 3 and proteins with at least 30%-99.5% sequence identity thereto. Accordingly, provided herein are proteins comprising the amino acid sequence of SEQ ID NO: 3 and proteins with at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto. Also provided herein are nucleic acids encoding the proteins comprising the amino acid sequence of SEQ ID NO: 3 and proteins with at least 30%-99.5% sequence identity thereto.

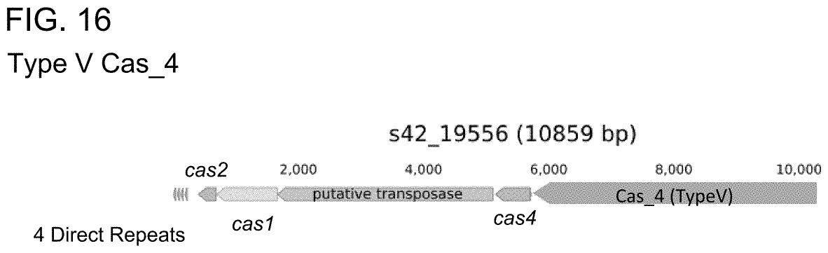

SEQ ID NO: 4 represents a novel Type V variant of the disclosure, Type V Cas_4, (1336 amino acids in length). FIG. 16 is a schematic representation of the organization of the CRISPR Cas cluster loci around the Type V Cas_4 gene of the disclosure. The loci has 4 direct repeats. FIG. 18 shows the amino acid sequence of Type V Cas_4 (SEQ ID NO: 4) with the RuvC motifs underlined/highlighted. The Fn Cas12a sequence referenced in Shmakov et al., 2015 was used as a reference for identification of the Ruv motifs. The RuvC I, II and III motifs are sequentially shown (highlighted in gray, with the conserved catalytic amino acids underlined)

In some embodiments the Type V CRISPR-Cas RNA-guided endonuclease of the disclosure comprises the amino acid sequence of SEQ ID NO: 4 and proteins with at least 30%-99.5% sequence identity thereto. Accordingly, provided herein are proteins comprising the amino acid sequence of SEQ ID NO: 4 and proteins with at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto. Also provided herein are nucleic acids encoding the proteins comprising the amino acid sequence of SEQ ID NO: 4 and proteins with at least 30%-99.5% sequence identity thereto.

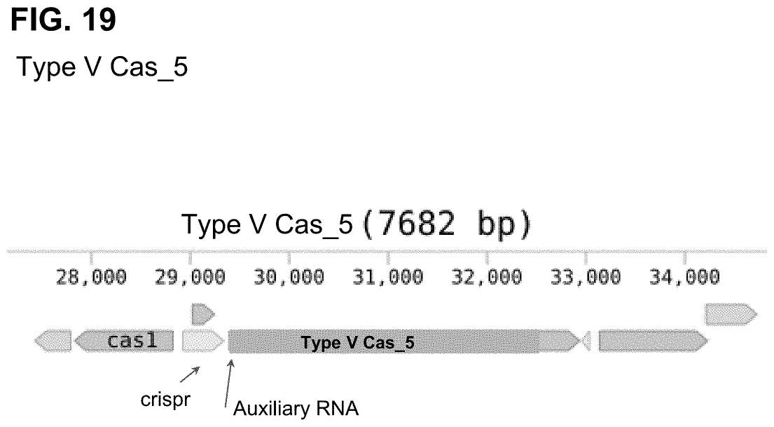

SEQ ID NO: 5 represents a novel Type V variant of the disclosure, Type V Cas_5, (1146 amino acids in length). FIG. 19 is a schematic representation of the organization of the CRISPR Cas cluster loci around the Type V Cas_5 gene of the disclosure. FIG. 21 shows the amino acid sequence of Type V Cas_5 (SEQ ID NO: 5) with the RuvC motifs underlined/highlighted. The RuvC I, II and III motifs are sequentially shown (highlighted in gray, with the conserved catalytic amino acids underlined). The Cas sequences from Chen et al. 2019 were used as a reference to deduce the RuvC motifs.

In some embodiments the Type V CRISPR-Cas RNA-guided endonuclease of the disclosure comprises the amino acid sequence of SEQ ID NO: 5 and proteins with at least 30%-99.5% sequence identity thereto. Accordingly, provided herein are proteins comprising the amino acid sequence of SEQ ID NO: 5 and proteins with at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto. Also provided herein are nucleic acids encoding the proteins comprising the amino acid sequence of SEQ ID NO: 5 and proteins with at least 30%-99.5% sequence identity thereto.

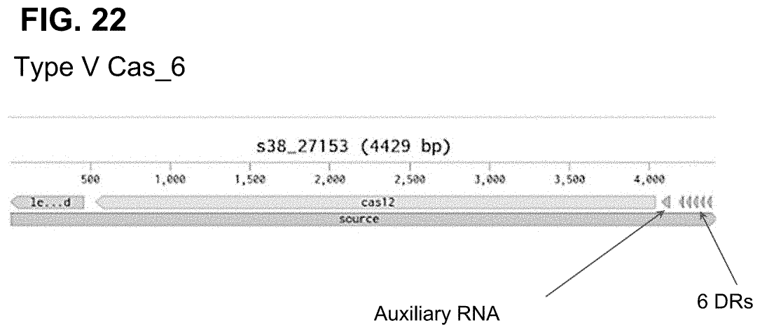

SEQ ID NO: 6 represents a novel Type V variant of the disclosure, Type V Cas_6, (1167amino acids in length). FIG. 22 is a schematic representation of the organization of the CRISPR Cas cluster loci around the Type V Cas_6 gene of the disclosure. The loci has 6 direct repeats, and a auxiliary RNA. FIG. 24 shows the amino acid sequence of Type V Cas_6 (SEQ ID NO: 6) with the RuvC motifs underlined/highlighted. The RuvC I, II and III motifs are sequentially shown (highlighted in gray, with the conserved catalytic amino acids underlined). The Cas sequences from Chen et al. 2019 were used as a reference to deduce the RuvC motifs.

In some embodiments the Type V CRISPR-Cas RNA-guided endonuclease of the disclosure comprises the amino acid sequence of SEQ ID NO: 6 and proteins with at least 30%-99.5% sequence identity thereto. Accordingly, provided herein are proteins comprising the amino acid sequence of SEQ ID NO: 6 and proteins with at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto. Also provided herein are nucleic acids encoding the proteins comprising the amino acid sequence of SEQ ID NO: 6 and proteins with at least 30%-99.5% sequence identity thereto.

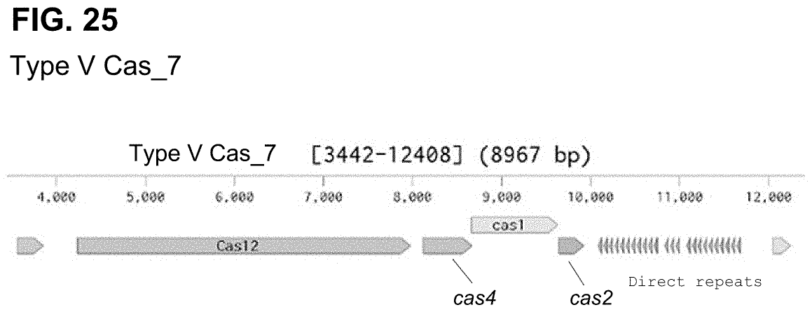

SEQ ID NO: 7 represents a novel Type V variant of the disclosure, Type V Cas_7, (1245 amino acids in length). FIG. 25 is a schematic representation of the organization of the CRISPR Cas cluster loci around the Type V Cas_7 gene of the disclosure. FIG. 27 shows the amino acid sequence of Type V Cas_7 (SEQ ID NO: 7) with the RuvC motifs underlined/highlighted. The RuvC I, II and III motifs are sequentially shown (highlighted in gray, with the conserved catalytic amino acids underlined). The FnCas12a sequence referenced in Shmakov et al., 2015 was used as a reference for identification of the Ruv motifs.

In some embodiments the Type V CRISPR-Cas RNA-guided endonuclease of the disclosure comprises the amino acid sequence of SEQ ID NO: 7 and proteins with at least 30%-99.5% sequence identity thereto. Accordingly, provided herein are proteins comprising the amino acid sequence of SEQ ID NO: 7 and proteins with at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto. Also provided herein are nucleic acids encoding the proteins comprising the amino acid sequence of SEQ ID NO: 7 and proteins with at least 30%-99.5% sequence identity thereto.

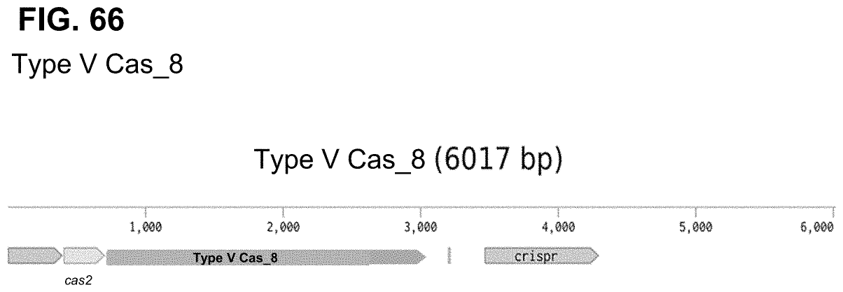

SEQ ID NO: 20 represents a novel Type V variant of the disclosure, Type V Cas_8, (758 amino acids in length). FIG. 66 is a schematic representation of the organization of the CRISPR Cas cluster loci around the Type V Cas_8 gene of the disclosure. FIG. 68 shows the amino acid sequence of Type V Cas_8 (SEQ ID NO: 20) with the RuvC motifs underlined/highlighted. Probable catalytic residues are D418, E597, D696 (depicted in bold and underlined/highlighted) and D481. The RuvC I, II and III motifs are sequentially shown (highlighted in gray with the conserved catalytic amino acids underlined). The Type V Cas sequences from Harrington et al. 2018 were used as reference for Ruv motifs search.

In some embodiments the Type V CRISPR-Cas RNA-guided endonuclease of the disclosure comprises the amino acid sequence of SEQ ID NO: 20 and proteins with at least 30%-99.5% sequence identity thereto. Accordingly, provided herein are proteins comprising the amino acid sequence of SEQ ID NO: 20 and proteins with at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto. Also provided herein are nucleic acids encoding the proteins comprising the amino acid sequence of SEQ ID NO: 20 and proteins with at least 30%-99.5% sequence identity thereto.

Table 3 provides exemplary nucleic acid sequences for encoding certain Type V sequences of the disclosure. Also provided are exemplary codon optimized nucleic acid sequences for encoding certain Type V sequences of the disclosure, for production in E. Coli systems.

Accordingly, provided herein are exemplary nucleic acid sequences encoding the Type V CRISPR-Cas RNA-guided endonucleases of the disclosure. In some embodiments, a Type V CRISPR-Cas RNA-guided endonuclease is encoded by a nucleic acid sequence comprising or consisting of the sequence of any one of SEQ ID NOs: 21-34 and SEQ ID NOs 59-60, or a nucleic acid sequence with at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

TABLE 3

| | | CODON OPTIMIZED NUCLEIC ACID |
|---|---|---|
| NAME | NUCLEIC ACID SEQUENCE | SEQUENCE |
| Type V | ATGGAAGAAAATAGAAGTCAAAAAAAATGCATATG | ATGGAGGAAAACCGTAGCCAGAAGAAATGCATCTGGG |
| Cas_1 | GGATGAATTAACAAACGTTTATTCAGTATCAAAAAC | ACGAGCTGACCAACGTGTACAGCGTTAGCAAAACCCT |
| | TCTGCGTTTTGAATTAAAACCATTAGGAGAAACCT | GCGTTTCGAGCTGAAGCCGCTGGGTGAAACCCTGAAA |
| | TGAAAAATATTAGGAAAAAAGGCTTGATAGAAGAA | AACATTCGTAAGAAAGGCCTGATCGAGGAAGATAAGA |
| | GATAAAAAAAGAGACGAAGATTTTTTAGAAGTGAA | AACGTGACGAAGACTTCCTGGAAGTGAAGAAGATCAT |
| | AAAAATAATTGATAAATATCTAAGTTATTTTATTGAT | TGACAAATACCTGAGCTATTTCATTGATCGTAACCTGG |
| | AGAAATTTAGATGGTTCTAAAAACTTAATTGAAGAA | ACGGCAGCAAGAACCTGATCGAGGAACACCAGCTGAA |
| | CATCAATTGAAAGAAATACAAGATATTTATGAAAAA | AGAGATCCAAGATATTTACGAAAAGCTGAAGAAAAACA |
| | CTAAAGAAAAATACTACTGATGAAAACTTGAAGAA | CCACCGATGAGAACCTGAAGAAAGACTATGCGAGCCT |
| | AGATTATGCTTCTTTACAAAGTAAATTAAGAAAAGA | GCAGAGCAAACTGCGTAAGGAAATCTTTGCGCAACTG |
| | AATTTTTGCTCAACTGAAAACAAAAGGCCATTATAA | AAGACCAAAGGTCACTACAAGGATTTCTTTGGCAAACA |
| | AGATTTTTTTGGAAAGCAATTTATTAAAAAAGTTTT | GTTCATTAAGAAAGTTCTGCTGGACTACTATAAGGAAG |
| | ATTAGATTATTATAAAGAAGAAGATAACAAATATGA | AGGACAACAAATATGACCTGCTGAAGAAATTTGAAAAC |
| | TTTATTAAAAAAATTTGAAAATTGGAATACTTATTTT | TGGAACACCTACTTCACCGGTTTCTACGAGAACCGTAA |
| | ACAGGATTTTATGAAAATAGAAAAAATATTTTTACC | GAACATCTTCACCGAAAAGGACATCAGCACCAGCCTG |
| | GAAAAGGATATTTCAACTTCTTTAACTTATAGAATT | ACCTACCGTATTGTGAACGATAACCTGCCGAAATTTCT |
| | GTAAATGATAATTTGCCAAAATTTTTAGATAATATT | GGACAACATCGCGAAGTATAACGAGCTGAAAAACAGC |
| | GCAAAATACAATGAACTAAAAAATAGTCTTCCTATT | CTGCCGATCCAGGAAATTGAGGAAGAGTTCAAGGATT |
| | CAAGAGATAGAAGAAGAGTTTAAAGATTATTTACA | ACCTGCAAGGCATGCCGCTGAACGTTTTCTTTAGCCT |
| | AGGAATGCCCTTAAATGTATTTTTTAGTTTAAGTAA | GAGCAACTTCAAAAACTGCCTGAACCAGAAGGGCATT |
| | TTTTAAAAATTGCTTGAATCAGAAGGGAATAGATA | GATACCTTTAACCTGCTGATCGGTGGCCGTAGCCCGG |
| | CTTTTAATTTATTAATTGGCGGAAGAAGTCCTGAT | ACGGCGAGAAGAAAATTAAAGGCCTGAACGAATACAT |
| | GGTGAGAAAAAAATTAAAGGATTGAATGAATATAT | CAACGAGCTGAGCCAACACAGCAACGACCCGAAAAGC |
| | CAATGAACTATCTCAACATAGTAATGATCCTAAATC | ATTAAGCGTCTGAAAATGATGCCGCTGTTCAAACAGAT |
| | TATAAAAAGACTTAAGATGATGCCTTTATTTAAGCA | CCTGGGCGAAAACAACACCAACAGCTTCCAATTTGAAA |
| | GATTTTAGGGGAGAATAATACTAATTCATTTCAATT | AGATCGAGTACGACCGTGATCTGATCAACCGTATTGA |
| | TGAAAAAATAGAATATGATAGAGATCTCATAAATAG | CGATTTTAACAAACGTCTGGAAGAGCAGGATCTGTACA |
| | AATTGATGATTTTAATAAAAGATTAGAGGAACAAGA | GCAACCTGTATGAGATCTTCAAGGACCTGAAAGACAA |
| | TTTATACTCTAATTTATATGAAATTTTTAAAGATTTG | CGATCTGCGTAAGATCTACATCAAGAACGGCAAGGAC |
| | AAAGATAATGATTTGAGAAAGATATATATTAAAAAT | ATCACCAACATTAGCCAGCAACTGTTTGGTGACTGGG |
| | GGTAAAGACATAACAAATATATCACAACAATTATTT | ATAAGCTGTACAAAGGCCTGCGTGAATATGCGGAGCA |
| | GGGGATTGGGACAAATTATATAAAGGTCTAAGAGA | AGACCTGTTCAGCCGTAAGAACGAAATCGAGAAATGG |
| | ATATGCAGAACAAGATTTATTTTCAAGAAAGAATGA | CTGAAGCGTAAATACATCAGCATTCACGAACTGGAGAA |
| | AATAGAGAAGTGGCTAAAAAGAAAATATATTTCAAT | AGCGATCGAGAAGCTGAAAATTAGCCAGGAATTTGAC |
| | TCATGAATTAGAAAAAGCAATTGAAAAATTAAAAAT | AAGAAACTGTACGAAAACTATCTGGAGAAGATTAACTA |
| | TAGTCAAGAATTTGATAAAAAATTATATGAAAATTA | TAACGAGAACAACCCGATCTGCGGCTTCCTGAGCACC |
| | TTTAGAAAAAATTAATTATAACGAAAACAATCCTAT | TTTAAGCAAAAAGAGAAGGATCTGCTGGAAGACATTAA |
| | TTGTGGTTTTCTATCTACTTTCAAACAAAAAGAGAA | AACCAACTACAGCAACTACCTGGAGATCAGCAAGAAG |
| | AGATTTGTTAGAAGATATAAAAACAAATTATTCCAA | GAGTTCGGCGAGGGCGACCTGCTGAAAGAGGACTAC |
| | TTATTTGGAAATATCAAAAAAAGAATTTGGTGAGG | CAGCGTGACGTGGAAATCATTAAGAGCTATCTGGATA |
| | GGGATTTGTTAAAAGAAGATTACCAAAGAGATGTT | GCCTGAAAGAGCTGCTGCACTACATCAAGCCGCTGTA |
| | GAAATAATTAAATCTTATTTGGATTCTCTAAAAGAG | TGTGGACAGCAAAGATACCGAAGACAGCAAGCAGCAA |
| | CTTTTACATTATATAAAACCACTCTATGTTGATAGC | GAAGTTTTTGAGCTGGACGCGAACTTCTACGAAACCTT |
| | AAAGACACAGAAGATTCGAAACAACAAGAAGTATT | TAACGAGCTGTATTTCGAACTGAAAGAGATCATTCCGC |
| | TGAGCTTGATGCTAATTTTTATGAAACATTTAATGA | TGTACAACAAAGTGCGTAACTATGTTACCCAAAAACCG |
| | ATTATATTTTGAATTAAAAGAAATAATCCCTCTTTAT | TTTAGCACCAAGAAATTCAAGCTGAACTTTGAGAACAG |
| | AATAAAGTAAGAAATTATGTAACTCAAAAACCTTTT | CACCCTGCTGAACGGTTGGGATAAAAACAAGGAACGT |
| | AGTACAAAGAAGTTTAAGTTAAATTTTGAAAACTCA | GACAACTTCAGCGTGATCCTGCGTAAGAAAAACGAGC |
| | ACATTACTAAATGGTTGGGATAAGAACAAAGAAAG | TGGGCACCTACGAATATTTCCTGGGTATTATGAGCCGT |
| | AGATAATTTTTCAGTAATTTTGAGAAAGAAAAATGA | GGCAACAACAAGATCTTTGAGAACATTGAAGAGAGCA |
| | ATTAGGAACTTACGAATATTTTTTAGGTATAATGTC | ACGAGGACGATAGCTTCGAAAAGATGGATTACAAACT |
| | TAGAGGAAATAATAAAATCTTTGAAAACATAGAAG | GCTGCCGGGTCCGGACAAGATGCTGCCGAAAGTTTTC |
| | AAAGTAATGAGGATGATTCTTTTGAAAAAATGGATT | TTTAGCGAGAAAAACATCAGCTACTATAAGCCGAGCGA |
| | ATAAATTACTTCCTGGCCCAGATAAAATGTTGCCT | AGACATCCTGGCGATTCGTAACCACAGCAGCCACACC |
| | AAAGTATTTTTTTCTGAAAAAAATATTAGTTATTATA | AAAAACGGTAGCCCGCAGGAAGGTTTCATGAAGAAAG |
| | AACCCTCAGAAGACATATTGGCTATTAGAAATCAT | AATTTAACAAGGACGATTGCCACAAAATGATTGATTTC |
| | TCCTCTCATACTAAAAATGGTTCTCCTCAAGAAGG | TACAAGAACGCGCTGAGCATCCACCCGGAGTGGAGCA |
| | TTTCATGAAAAAAGAATTTAATAAAGATGATTGTCA | ACTTCGAATTTAACTTCAAGAAAACCAGCTTTTACGAA |
| | TAAAATGATAGATTTTTATAAAAATGCATTATCTATT | GATACCAGCGAGTTCTTTAAAGACATCGCGGACCAGG |
| | CATCCTGAGTGGTCAAATTTTGAGTTTAATTTTAAA | GTTATCAAATCAACTTCCGTAACATTAGCAGCAAGGAC |
| | AAAACCTCCTTTTATGAAGATACTTCTGAATTTTTC | ATCAACCAGCTGGTTGACGAGGGCAAACTGTACCTGT |
| | AAAGATATAGCTGATCAAGGCTACCAAATCAATTT | TCCAAATCTATAACAAGGACTTTAGCACCAACAAGAGC |
| | CAGAAACATTTCTTCAAAAGATATTAATCAATTAGT | CAGAAAAACCGTAACAGCCGTAAAAACCTGCACACCC |
| | AGATGAAGGAAAATTATATTTGTTCCAAATATATAA | TGTACTGGGAAGAGCTGTTCAGCCCGGAAAACCTGCG |
| | TAAGGATTTTTCAACTAATAAATCTCAAAAAAATAG | TGATGTGGTTTATAAGCTGAACGGCGAAGCGGAGATT |
| | AAATAGTAGAAAAAATCTTCATACTCTATATTGGGA | TTCTTTCGTGAAAAGAGCATCGAGCCGAAAACCGAAC |
| | AGAATTATTTTCTCCTGAAAATCTTAGAGATGTTGT | ACCCGAAGAACCAAGAGATTAAAAACAAGGACCCGAT |
| | TTATAAGTTAAATGGGGAAGCTGAAATATTTTTCAG | CAACGGTAAGAAATACAGCAAGTTCAGCTATGATCTGA |
| | AGAGAAATCTATTGAGCCTAAAACAGAACACCCCA | TCAAAGACAAGCGTTACACCGAAGACAAGTTTCTGTTC |
| | AAAATCAAGAAATTAAAAATAAGGACCCAATTAATG | CACTGCCCGATTACCATGAACTTCAAAGCGAAGGGTA |
| | GAAAAAAATATAGTAAATTCTCTTATGATTTAATAA | GCAAATGGGACATCAACAAGATTGTGAACAGCACCATT |
| | AAGATAAAAGATATACTGAAGATAAATTTTTATTTC | AAGGAGAACAGCAAAGAAATCAACATTCTGAGCATCG |
| | ATTGTCCTATCACAATGAATTTCAAAGCAAAAGGTT | ACCGTGGTGAGCGTCACCTGGCGTACTGGACCCTGCT |
| | CAAAATGGGATATAAATAAAATTGTCAATAGTACAA | GAACAGCAAAGGCGAAATCGTTGACCAGGATAGCTTC |
| | TTAAAGAAAATTCAAAAGAAATTAATATATTGAGTA | AACATCATTAAAGAGGAAACCATTGGTCGTAAGACCGA |
| | TTGATAGAGGTGAGAGACATCTTGCATATTGGACT | TTATCACGAGAAGCTGAGCGAAAAAGAGGGCGACCGT |
| | TTATTAAATTCTAAAGGAGAAATTGTAGACCAAGAT | GATGAGGCGCGTAAGAACTGGAAGAAAATCGAAAACA |
| | TCTTTTAATATAATTAAAGAAGAGACTATTGGAAGA | TCAAGGAACTGAAAGAGGGCTACCTGAGCCAAGTGGT |
| | AAAACAGATTATCATGAAAAATTATCTGAAAAAGAA | TCACAAGCTGGCGAAACTGGCGGTGGAAGAGAACGC |
| | GGAGATAGGGATGAAGCCAGAAAGAATTGGAAGA | GATCATTGTTTTTGAGGACCTGAACTATGGTTTCAAAC |
| | AGATTGAAAATATTAAAGAATTAAAAGAAGGGTATT | GTGGCCGTTTTAAGATCGAAAAGCAGGTTTACCAAAAG |
| | TATCTCAAGTAGTTCATAAACTTGCAAAATTAGCAG | TTCGAGAAAATGCTGATCGAAAAGTTCAACTATCTGAT |
| | TTGAAGAAAATGCAATTATTGTTTTTGAGGATTTAA | GTTTAAGGATCGTGAGAAGAACGAGATTGCGGGTAGC |
| | ACTATGGTTTTAAACGAGGAAGATTTAAAATTGAG | CTGAACACCCTGCAGCTGACCCCGCAAATCAGCAGCG |
| | AAGCAAGTATATCAAAAATTTGAGAAAATGTTAATT | AAAAAGAGAAGGGTCGTCAGACCGGCGTGATCTTCTA |
| | GAAAAATTCAATTATCTAATGTTTAAAGATAGAGAA | CACCGATCCGAACTATACCAGCAAGATTGACCCGAAA |
| | AAAAATGAGATTGCAGGTTCATTAAACACTCTACA | ACCGGTTTCATCAACCTGCTGTACCCGAAATATGAAAG |
| | ATTAACGCCTCAAATAAGTTCAGAAAAAGAAAAAG | CGTTGAGAAAAGCAAGAACTTCTTTAAGAAGTTTGAGA |
| | GTAGACAAACAGGAGTAATATTTTATACTGATCCT | GCATCAAGTACAACGGCGAATATTTTGAGTTCACCTTT |
| | AATTATACATCAAAGATAGATCCTAAAACAGGTTTT | AACTACAGCAACTTCTATAACGATCTGAACCTGACCAA |
| | ATTAATTTATTATATCCCAAATATGAATCAGTTGAG | GAAAGAATGGACCATTTGCAGCTACGGTGACCGTATC |
| | AAATCAAAGAATTTTTTCAAAAAATTTGAATCAATT | TTCAGCTTTCGTAACCCGGAGAAAAACAACCAGTTTGA |
| | AAATATAATGGAGAATATTTTGAATTTACTTTTAATT | TACCAAGACCATCTACCCGACCGATGAACTGAAAAGC |
| | ATTCTAATTTTTATAATGATTTAAATTTAACAAAAAA | CTGTTCGACAAGTACTATATTGAATATGAGAGCCAGAA |
| | AGAGTGGACAATTTGTTCATATGGCGATAGGATTT | AAACATCCTGAACGAGATTACCAAGCAAAGCAGCAGC |
| | TCTCTTTTAGAAATCCTGAAAAAAATAATCAATTTG | GACTTCTACAAAAGCCTGATGTTTATCCTGAGCAAGAT |
| | ACACTAAAACAATTTATCCAACAGATGAACTGAAAT | TCTGCAACTGCGTAACAGCATCCCGAACAGCGAAGAG |
| | CATTGTTTGATAAATATTATATTGAATATGAAAGTC | GATTTCATCCTGAGCTGCATCAAGGATAAGAAAGGTAA |
| | AAAAAAATATTTTAAATGAAATAACCAAACAAAGTT | CTTCTTTGACAGCCGTAACGCGAACAAGAACACCGAG |
| | CAAGTGATTTTTACAAATCATTAATGTTTATTTTAA | CCGGCGAACGCGGACAGCAACGGTGCGTACAACATC |
| | GTAAGATATTACAATTAAGAAATTCTATACCAAATT | GGTATTAAAGGCCTGATGATCATTGAGCGTATCAAGAA |
| | CCGAAGAAGATTTTATCTTGTCATGTATAAAAGATA | CTGCCCGGAAGATAAGAAACCGAACCTGACCATTAAA |
| | AAAAAGGTAATTTCTTTGATTCAAGAAATGCTAATA | CGTGACGAGTTCGTGAACTATGTTATCGGTCGTAACAC |
| | AAAACACAGAACCTGCAAATGCAGATTCAAACGGA | CTAG (SEQ ID NO: 22) |
| | GCTTATAATATTGGAATAAAAGGTTTAATGATAATT | |
| | GAGAGAATTAAAAATTGTCCAGAAGATAAAAAACC | |
| | TAATTTAACAATTAAGAGGGATGAATTTGTGAATTA | |
| | TGTAATAGGGAGGAATACATAG (SEQ ID NO: | |
| | 21) | |
| Type V | ATGATAAATATTGACGAATTAAAAAATTTATATAA | ATGATTAACATCGACGAACTGAAAAACCTGTATAAAGT |
| Cas_2 | AGTTCAAAAAACAATTACTTTTGAATTAAAAAATA | GCAGAAGACCATCACCTTTGAACTGAAAAACAAGTGG |
| | AATGGGAAAATAAGAATGATGAAAATGATAGAGT | GAAAATAAGAATGACGAGAACGATCGTGTGGAGTTCCT |
| | TGAGTTTTTAAAGACTCAAGAATGGGTGGAATCTT | GAAGACCCAGGAGTGGGTGGAAAGCCTGTTCAAAGTT |
| | TATTCAAAGTTGATGAGGAGAATTTTGATGAAAAG | GATGAGGAAAACTTTGACGAGAAGGAAAGCATTCCGA |
| | GAGTCAATTCCGAACTTGTTAGATTTCGGCCAAAA | ACCTGCTGGACTTCGGTCAAAAGATCGCGAGCCTGTTT |
| | GATTGCGAGTCTTTTTTATAAGTTGAGTGAAGATAT | TATAAACTGAGCGAAGATATTGCGAACAACCAGATCGA |
| | CGCTAATAATCAAATTGATACACGGGTTTTAAAAG | CACCCGTGTGCTGAAGGTTAGCAAATTTCTGCTGGAGG |
| | TGAGCAAGTTTTTGTTGGAGGAGATCGATAGAAAT | AAATTGATCGTAACCAATACCACGAGAAGAAAAACAA |
| | CAATATCATGAGAAAAAAAATAAACCAACAAAGG | ACCGACCAAGGTGAAAGAAATGAACCCGAACACCAAC |
| | TTAAGGAGATGAATCCAAATACAAATAAGAGTTAT | AAGAGCTATATTAAGGAGTACAAACTGAGCGATCAGA |
| | ATTAAGGAGTATAAGTTATCAGATCAAAATACATT | ACACCCTGTACGTTCTGCTGAAGATCATGGAGGACGAA |
| | GTATGTTCTGTTGAAGATAATGGAAGATGAAGGGC | GGTCGTGGCCTGCAAAAATTCCTGTATGATAAGGCGGA |
| | GGGGTTTACAAAAATTTTTATATGATAAGGCAGAC | CCGTCTGAACCTGTACAACCAGAAAGTTCGTCGTGACT |
| | AGATTAAATTTATATAATCAGAAGGTAAGAAGAGA | TCGCGCTGAAGGAGAGCAACGAACAGCAAAAATTTAG |
| | TTTCGCTTTAAAAGAAAGTAACGAACAGCAGAAGT | CGGTAACGCGAACTACTATGGCAACATTAAACTGCTGA |
| | TTTCGGGTAACGCTAATTATTACGGAAACATAAAA | TCGATAGCCTGGAGGACGCGGTGCGTATCATTGGTTAT |
| | TTGTTGATTGATTCATTGGAAGACGCTGTTCGTATT | TTCACCTTTGACGATCAAGCGGAGAACGCGCAGATCAA |
| | ATTGGTTATTTCACGTTTGATGATCAAGCAGAAAAT | CGAGTTCAAGAGCGTTAAACAAGAGATGAACAACAAC |
| | GCTCAAATAAATGAATTCAAGAGCGTTAAGCAGGA | GAAGCGAGCTACCAGGCGCTGAAAGATTTTGCGATTGA |
| | AATGAATAACAATGAAGCTTCGTATCAGGCTTTGA | CAACGCGAAGAAAGAGATCGAACTGACCACCCTGAAC |
| | AAGATTTTGCTATTGATAACGCAAAAAAAGAAATT | CACCGTGCGGTGAACAAGGACCCGAAGAAGATCCAAG |
| | GAACTTACAACTCTAAATCATAGGGCTGTTAACAA | AGCAGATCGAGGAAGTTGAAAACTTCGAGGAAGACAT |
| | GGATCCAAAAAAGATACAAGAACAGATTGAAGAA | TAACCAACTGAAACACCAGATCAGCGCGCTGAACGATA |
| | GTGGAAAATTTTGAAGAAGATATAAATCAATTGAA | AGAAATTTGACGTGGTTAGCCGTCTGAAACACGCGCTG |
| | GCACCAAATTTCTGCGCTTAATGATAAAAAATTTG | ATTAAGATGCTGCCGGAGCTGAACCTGCTGGATGCGGA |
| | ATGTAGTGTCAAGATTAAAGCATGCATTAATTAAA | GAGCGAACAAGGTCGTGAAGTGCAGCAAATCTACCAG |
| | ATGTTACCGGAGTTGAATTTGTTAGATGCTGAAAG | GACAAGAAAAACGGCCTGGAGCTGGACGATTTCAAATT |
| | CGAGCAAGGTAGAGAGGTTCAGCAAATATATCAA | TAACCTGCTGAAGCACCACCAATGGCAGAAAACCATTT |
| | GATAAAAAGAATGGTTTGGAATTAGACGATTTTAA | TCAAGTATATCAAACTGGAGGGTCTGGTGCTGCCGGAT |
| | GTTCAATTTGCTTAAACATCATCAATGGCAGAAAA | CTGTACGCGGAAAACAAGCAAGACAAGATCAAGGTTT |
| | CCATTTTTAAATACATTAAATTAGAGGGTTTGGTTT | ACATCGAGAACTACCGTCAGAGCGGCGAACGTATTAGC |
| | TACCTGATTTATATGCCGAAAACAAACAAGATAAG | AAGAAAGCGCGTGAGGAACTGGGCAAGATCGATAAAC |
| | ATTAAAGTGTATATTGAAAATTATCGACAAAGCGG | GTGAGGAGTTCAACGGCAACGACGAGCTGAAGAAAGC |
| | AGAAAGGATAAGTAAAAAGGCACGCGAGGAGTTG | GTGGTATGAATACAAGGATTTTTGCCGTGACAAGCGTA |
| | GGCAAGATCGATAAAAGAGAGGAATTTAATGGTA | ACAAAAGCGTGGAACTGGGTAACAAGAAAAGCCTGTA |
| | ATGATGAACTAAAGAAAGCGTGGTACGAATACAA | CAACGCGATCAAGCGTGAGGTTCTGCGTCAGAAAATGT |
| | AGATTTTTGCAGAGACAAGCGTAATAAATCCGTGG | GCAACCACTTCGCGGTGCTGGTTAGCGATGGCGAGGAC |
| | AATTGGGCAATAAGAAATCACTGTACAATGCCATC | ACCAGCCCGTACTATTACCTGATCCTGATTCCGAACGA |
| | AAGCGTGAGGTTTTAAGGCAGAAAATGTGTAATCA | GAACAGCGATGAAATGAACCGTACCTTTAAGGAGCTGA |
| | TTTTGCCGTATTGGTGAGTGATGGGGAAGATACAT | AAGCGAGCGAGGGTAACTGGAAAATGCTGGACTACAA |
| | CGCCTTATTATTATTTGATATTAATTCCCAATGAAA | CCGTCTGACCTTCAAGGCGCTGGAAAAACTGGCGCTGC |
| | ACAGTGATGAAATGAACAGGACATTCAAAGAGCTT | TGCGTAGCAGCACCTTTGAGATTGCGGATCAAGAACTG |
| | AAAGCATCCGAAGGAAATTGGAAGATGCTCGATTA | CAGGAAGAGGCGAAGAAGATCTGGGAGGAATATAAGG |
| | TAACAGATTAACTTTTAAAGCTTTGGAAAAATTGG | AGAAAGCGTACAAGGACTTCAAGAACAAGAAACTGCT |
| | CATTATTGCGCAGCTCTACATTTGAAATTGCAGACC | GCAAGGTCTGAGCGGCCGTCAGCGTGAGGAAAAGAAA |
| | AAGAACTACAAGAAGAAGCTAAAAAAATTTGGGA | CAAGAGCTGCAGAAGGAAAGCCTGAACCGTGTGATCA |
| | AGAATATAAAGAAAAGGCGTATAAAGATTTTAAGA | ACTATCTGATCCGTTGCATTCAAAGCCGCCGGACAGC |
| | ATAAAAAATTATTACAAGGGCTATCCGGTCGCCAA | GGTAAATATAACTTCAACTTTAAGGAACCGCACCAATA |
| | AGAGAAGAAAAAAAACAAGAATTGCAAAAAGAAA | CCAGAGCCTGGAGGAGTTCGCGGAGGAAATTGATCGTC |
| | GTTTAAATCGAGTTATAAATTATTTAATTCGTTGCA | AGGGCTACCACTGCGCGTGGAAAAACGTTAGCAAGGA |
| | TTCAGTCGTTGCCGGATAGCGGTAAATACAATTTTA | CAAACTGATGGAGCTGGAAGCGATGGAGAAGATCAAA |
| | ATTTTAAAGAACCGCATCAATATCAGAGCTTGGAA | GTGTTCAAGCTGCACAACAAAGATTTTCGTAAGGTTAA |
| | GAGTTTGCGGAAGAAATTGATAGACAGGGTTATCA | ACTGAACGACAGCAAACACAACCCGAACCTGTTTACCC |
| | TTGCGCTTGGAAGAATGTAAGCAAAGACAAGCTTA | TGTATTGGCTGGATGCGATGAACCTGGACAAGGTGAAC |
| | TGGAGCTGGAGGCGATGGAAAAAATTAAAGTATTT | GTTCGTCTGCTGCCGGAAGTGGATCTGTACAAGCGTGC |
| | AAATTGCATAATAAGGATTTTAGAAAAGTTAAACT | GAAAGAAACCCAGCTGAAGCTGTTCGAACGTGACGTTA |
| | TAACGATTCGAAACACAATCCGAATCTTTTTACTTT | AATGCAACATCAACAACCAAAAGATCAAAAGCATTAA |
| | ATATTGGCTTGACGCGATGAATTTGGATAAAGTCA | GGAGAAAAACCGTCTGTTTCAGGATAAACTGTATGCGA |
| | ATGTTCGTTTATTGCCCGAGGTGGATTTATATAAAA | GCTTCAAGCTGGAGTTTTACCCGGAGAACGAAGGTCTG |
| | GAGCCAAAGAAACGCAACTAAAATTATTCGAAAG | GGCTTCGAACAGGTGAACGACAAGGTTAACAACTTTTG |
| | AGATGTAAAGTGCAATATTAATAATCAAAAAATAA | CGGTAGCGATACCGCGTATTACCTGGGTCTGGACCGTG |
| | AATCAATTAAAGAAAAAAATAGATTATTTCAAGAT | GCGAGAAAGAACTGGTGACCTTCTGCCTGGTTGACAGC |
| | AAACTTTACGCTTCATTCAAGCTGGAATTTTATCCA | GATGGTCGTCTGGTGAAGAACGGCGATTGGACCAAGTT |
| | GAAAACGAAGGTTTGGGTTTTGAACAAGTCAATGA | CAAAGAAGTTAACTATGCGGACAAGCTGAAACAATTTT |
| | TAAAGTGAATAATTTTTGCGGAAGTGATACAGCGT | ATTACAGCAAAGGCGAGATTGAAAGCACCCAGCAACA |
| | ATTATTTGGGTTTGGATAGGGGTGAGAAAGAATTG | GCTGCTGGAGGCGCGTGATAACATCAAGCAGGCGACC |
| | GTTACGTTTTGCTTGGTTGATTCTGATGGGCGGTTG | AACACCGAGGACAAGGAAAGCATGAAACTGAACTACA |
| | GTTAAGAACGGAGATTGGACGAAGTTTAAAGAGGT | AGAAACTGGAGCTGAAGCTGAAACAACAGAACCTGCT |
| | TAACTATGCGGATAAATTAAAGCAATTTTATTATTC | GGCGCAGGAATTTATTAAGAAAGCGTATTGCGGTTACC |
| | AAAAGGTGAAATAGAATCTACTCAACAACAACTTT | TGATCGATAGCATTAACGAGATCCTGCGTGAATATCCG |
| | TGGAAGCTCGAGACAATATTAAACAAGCTACTAAC | AACACCTACCTGGGCTGGAAGACCTGGATATCGCGGG |
| | ACGGAGGATAAAGAATCGATGAAATTAAACTATAA | TAAAGCGGACCCGGAGAGCGGCATGACCAACAAAGAA |
| | AAAATTAGAGTTGAAACTAAAACAACAGAATTTGT | CAAAACCTGAACAAGACCATGGGTGCGAGCGTTTATCA |
| | TAGCGCAGGAGTTTATTAAAAAAGCTTATTGCGGT | GGCGATTGAGAACGCGATCGTGAACAAGTTCAAATACC |
| | TATTTGATAGATTCAATAAATGAAATATTACGGGA | GTACCGTTAAACTGAGCGACATTAAGGGCCTGCAAACC |
| | ATATCCAAATACGTATCTTGTATTAGAGGATTTGGA | GTGCCGAACGTGGTTAAGGTTGAGGATCTGCGTGAAGT |
| | TATAGCAGGTAAAGCTGACCCCGAAAGCGGCATGA | GAAAGAGGTTGAAGACGGCGAGCACAAGTTCGGCCTG |
| | CCAATAAAGAACAAAATTTAAATAAAACAATGGGT | ATCCGTAGCGTGAAGAGCAAAGATCAGATTGGTAACAT |
| | GCCAGCGTTTATCAAGCTATTGAAAATGCCATAGT | CCTGTTTGTTGACGAGGGCGAAACCAGCAACACCTGCC |
| | AAATAAGTTTAAATACCGTACTGTTAAATTATCCG | CGAACTGCGGCTTCAACAGCGATTGGTTTAAACGTGAC |
| | ATATCAAAGGTTTGCAAACTGTACCGAATGTAGTG | GTGGATTTCGACCTGGAAATTGTGGCGACCGTTAACGG |
| | AAGGTGGAAGATTTGCGCGAAGTTAAGGAAGTGG | TCAAAAGAACGCGGTTATCGAGCAGAACGACAAGAAA |
| | AAGATGGTGAGCATAAATTTGGTTTGATAAGATCC | TATTGCTTTCCGGGCGAGATCTACAAACTGGAAATCAT |
| | GTGAAATCAAAGGATCAAATTGGCAATATTCTGTT | TAACAAGGAGTACGAAACCAACAAACGTAACCTGGCG |
| | TGTGGATGAAGGAGAAACATCTAATACTTGCCCGA | ATGATTTTCAAGCCGCGTGCGAAAGCGTGCCGTAAGTT |
| | ATTGCGGATTTAACAGCGATTGGTTTAAGCGGGAT | TATCAACAACAACCTGGATAAGAACGACTATTTCTACT |
| | GTTGATTTTGATTTGGAGATTGTGGCTACTGTAAAC | GCCCGTATTGCGCGTTTAGCAGCAAGAACTGCAACAAC |
| | GGTCAGAAAAATGCGGTTATAGAACAAAACGACA | CCGAAACTGCAGAACGGTGACTTCGTGGTTTACAGCGG |
| | AAAAGTACTGTTTTCCCGGTGAAATTTATAAGTTAG | CGACGATGTGGCGGCGTATAATGTGGCGATCCGTGGTA |
| | AAATAATTAATAAAGAATACGAAACAAATAAACG | TCAATCTGCTGAATAATATCAAGTAG (SEQ ID NO: |
| | GAATTTAGCCATGATTTTTAAACCGCGCGCAAAAG | 24) |
| | CTTGTAGAAAATTTATAAATAATAATTTGGATAAG | |
| | AATGACTATTTTTATTGCCCGTATTGCGCTTTTTCTA | |
| | GCAAGAACTGCAATAATCCAAAATTGCAAAACGGT | |
| | GATTTTGTGGTATATTCGGGTGATGATGTGGCGGC | |
| | ATACAATGTAGCGATCAGAGGTATTAACCTTTTAA | |
| | ACAATATAAAATAG (SEQ ID NO: 23) | |
| Type V | ATGCATCTATCTCAAACATTTACAAACAAATATCA | ATGCACCTGAGCCAGACCTTCACCAACAAGTACCAAGT |
| Cas_3 | GGTATCAAAAACATTAAGGTTTGAACTTAGGCCAC | GAGCAAAACCCTGCGTTTTGAGCTGCGTCCGCAGGGTC |
| | AAGGCCAAACCAAGGAAAAATTTGAAAGATGGAT | AAACCAAAGAGAAGTTCGAACGTTGGATCGCGGAGCT |
| | TGCTGAACTAAGAACAGAAAACCCAAGTGCTGATA | GCGTACCGAAAACCCGAGCGCGGATAACCTGATTGCGG |
| | ATTTAATCGCAGAAGATGAGCAAAGAGCAGTAGAT | AGGACGAACAGCGTGCGGTGGATTATAAGGAAGTTAA |
| | TATAAAGAAGTAAAAAGTATCATAGATCGTTTTCA | AAGCATCATTGACCGTTTTCACCGTAAGGTTATCGAGG |
| | TAGAAAAGTGATTGAAGAAAGTTTGGAGGGCTTGA | AAAGCCTGGAGGGTCTGAAACTGAAGGGCCTGAGCGA |
| | AGTTGAAAGGACTATCAGAATATGAGGAACTCTAT | ATATGAGGAACTGTACTTCAAGCGTGAGAAAGAAGAC |
| | TTTAAGCGTGAAAAAGAAGATATCGACCTTAAGGA | ATCGATCTGAAGGAGATTGAAAACCTGCAGATCCAAAT |
| | GATAGAAAATCTGCAAATACAAATGCGAAAGCAA | GCGTAAACAGATCCGTGAGGCGTTCGTGGAACACCCGG |
| | ATTAGAGAGGCATTTGTTGAACACCCTGTTTTTAAA | TTTTCAAGGACCTGTTTAAGAAAGAGCTGATCCAAGTG |
| | GATTTATTCAAAAAAGAATTGATTCAAGTTCATTTA | CACCTGAAAGAGTGGCTGACCGATCAGCAAGAAATTG |
| | AAAGAATGGCTTACGGATCAACAAGAGATTGATTT | ACCTGGTTGCGAAGTTCGAGAAATTTACCACCTACTTC |
| | GGTTGCCAAGTTTGAAAAATTCACCACCTACTTTG | GGTGGCTTTCACGAAAACCGTCAGAACGTGTATAGCCC |
| | GTGGTTTTCATGAGAATCGACAGAATGTCTATAGT | GGATGCGAAGGCGACCGCGGTTGGTTATCGTATGATCC |
| | CCGGATGCAAAAGCTACCGCAGTGGGCTACAGAAT | ACGAGAACCTGCCGAAATTCCTGGACAACCGTCGTATC |
| | GATTCATGAAAACTTGCCGAAGTTTTTAGACAATC | TTCAACAAGATCATCAAGGCGCACGAGGAACTGGATTT |
| | GAAGAATTTTTAATAAAATCATAAAAGCACATGAA | CAGCAGCATCGACAGCGAACTGGAGGAACTGCTGCAG |
| | GAGCTAGATTTCTCATCAATTGATTCAGAGTTAGA | GGCACCACCGTGGAGGAAGTTTTCAGCCTGGAGTTTTA |
| | AGAGCTTTTACAAGGAACTACTGTTGAGGAAGTTT | CAACGAAACCCTGACCCAAACCGGCATCGACATTTACA |
| | TTTCGCTAGAATTTTATAACGAAACACTGACGCAA | ACCACGTGCTGGGTGGCTATAGCAGCGAAACCGGTCAG |
| | ACCGGAATCGATATTTATAATCATGTATTGGGAGG | AAGATCCAAGGCGTTAACGAAAAAATTAACCTGTATCG |
| | CTATTCTTCTGAAACAGGACAAAAGATTCAGGGAG | TCAGAAGAACGGCCTGAAAGCGCGTGAGCTGCCGAAC |
| | TGAATGAGAAAATCAATTTGTACCGACAGAAGAAT | CTGAAGCCGCTGTTTAAACAGATCCTGAGCGAGAGCCA |
| | GGGTTAAAAGCCAGAGAGTTGCCCAACCTTAAGCC | AACCGCGAGCTTCGTGATCGAACAAATTGAGAGCGAA |
| | ATTATTCAAACAAATATTGAGTGAAAGTCAAACCG | AGCGACCTGCTGGATCGTCTGGACAACTTCCACACCCT |
| | CTTCTTTTGTCATAGAGCAAATAGAAAGTGAATCG | GATTACCAGCTTCGAGTTTCAGGGTCGTAACCAAGTGA |
| | GATTTATTAGACAGGCTAGACAATTTTCACACCCT | ACGTTATGACCGAACTGAAGCACATGCTGGCGGCGCTG |
| | AATAACAAGTTTCGAATTTCAAGGAAGAAATCAAG | GATAGCTATGAGCACGAACAGGTGTACTTTAAAAACGG |
| | TAAATGTAATGACCGAGCTCAAGCATATGTTAGCA | CCCGAGCCTGACCCAGCTGAGCCAAAAGATGTTCGGTC |
| | GCGCTAGATTCATATGAACATGAGCAAGTATATTT | AATGGGGCGTTATCCACAAAGCGCTGGAGTACTATTAC |
| | TAAAAATGGCCCAAGTCTTACTCAATTATCACAAA | GAGCAGGAACAAAACCCGCTGCAGGGTAAGAAACTGA |
| | AGATGTTTGGGCAATGGGGCGTGATTCATAAGGCA | CCAAGAAATACGAGAACGACAAAGAAAAGTGGCTGAA |
| | CTGGAATATTATTATGAGCAAGAGCAAAATCCTTT | AAACAAGCAGTTCAACCTGAGCCTGCTGCAAAAGGCG |
| | ACAAGGTAAGAAACTGACTAAAAAATATGAGAAT | ATCGATGTGTATGTTCCGACCATCGACACCATTGAGCC |
| | GATAAAGAGAAATGGTTAAAAAATAAACAGTTCA | GGTGAGCATTGTTGAAACCCTGAGCACCCTGGAGGATA |
| | ATTTGAGCCTTTTGCAGAAGGCAATAGATGTCTAT | AAGAAGGTGCTGACCTGGGCACCGAGGTGGATAACGC |
| | GTGCCAACGATCGATACCATAGAACCTGTCAGTAT | GTACGAAAAGGTTGCGGAGCTGATCGAACAGAAAACC |
| | AGTAGAAACACTTTCCACGTTAGAAGACAAAGAAG | CTGAGCGAAAGCTACGCGCAGAAGAAAAAGGAGAAGC |
| | GTGCAGATTTAGGTACGGAAGTGGATAATGCTTAC | AAGTGATCAAGGAATATCTGGACGGTCTGATGAGCCTG |
| | GAGAAAGTAGCTGAATTAATAGAGCAAAAGACATT | CTGCACAGCGTGAAGCCGTTCTATACCACCGAGGTTGA |
| | GAGTGAAAGCTACGCACAAAAAAAGAAGGAGAAG | CATCGAAAAAGACGCGGGTTTCTACGGCCTGTTTGAGC |
| | CAAGTCATTAAAGAATATCTCGATGGTTTAATGAG | CGCTGTATGAACAGCTGAACCTGGTGATCCCGATTTAT |
| | TCTTTTACATAGTGTAAAGCCTTTTTATACGACCGA | AACCTGGTTCGTAACTACCTGACCCAAAAACCGTATAG |
| | GGTTGATATAGAAAAAGATGCCGGATTTTACGGGT | CACCGAGAAATTCAAGCTGAACTTTGAAAACAACACCC |
| | TATTTGAACCGCTGTATGAGCAACTAAACCTAGTA | TGCTGGATGGTTGGGACCAGAACAAAGAGAAGGCGAA |
| | ATTCCTATTTATAATTTGGTGAGAAATTACCTCACA | CACCTGCGTTCTGCTGCGTAAGGAAGGCAACTATTACC |
| | CAAAAACCTTATTCAACTGAAAAATTTAAACTGAA | TGGCGGTGATGCACAAAAACCACAACACCGTTTTCGAG |
| | TTTTGAAAATAATACTCTTTTGGATGGTTGGGATCA | GAACTGCCGCAAAACGAGAACGCGACCTATGAAAAGG |
| | GAATAAAGAGAAGGCAAATACATGCGTATTATTAA | TGATCTACAAACTGCTGCCGGGTGCGAACAAGATGCTG |
| | GGAAAGAGGGTAATTATTATTTGGCGGTTATGCAC | CCGAAAGTTTTCTTTAGCAAAAAGAACATCGATTACTA |
| | AAAAATCACAACACGGTATTTGAAGAGCTGCCCCA | CAAGCCGAAAGAGGAGCTGCTGGAGAAATACAAGCTG |
| | AAATGAAAATGCGACTTATGAAAAAGTAATTTATA | GGCACCCACAAAAAGGGCAGCAACTTTAACCTGAAGG |
| | AACTTTTGCCTGGAGCCAATAAAATGTTACCCAAG | ACTGCCACGCGCTGATCGATTTCTTTAAGGACAGCATT |
| | GTTTTCTTTTCAAAAAAGAATATAGACTACTATAAA | AGCAAACACCCGGATTGGGCGCAGTTCAACTTTGAGTT |
| | CCCAAAGAAGAACTTTTAGAAAAATATAAGCTAGG | CAGCCAAACCAAAACCTACGAAGACCTGAGCCACTTCT |
| | CACTCATAAAAAGGGAAGTAATTTCAATCTCAAAG | ATCGTGAGGTGGAACACCAGGGCTATAAGATCAACTAC |
| | ACTGTCATGCGCTAATTGATTTTTTCAAGGACTCCA | GCGAAAGTGGATGTTAGCTACATTAACCAGCTGGTTGA |
| | TTTCCAAACATCCTGATTGGGCTCAATTCAATTTTG | CGATGGTCGTATTTTTCTGTTCCAAATCTACAACAAGGA |
| | AGTTTTCACAAACAAAAACCTATGAAGATTTAAGC | CTTTAGCCCGTATAGCAAAGGCAAGCCGAACCTGCACA |
| | CATTTTTACAGAGAAGTAGAGCATCAGGGATACAA | CCATGTACTGGCGTGCGGTGTTCGACGAGAAGAACCTG |
| | AATCAATTATGCAAAGGTTGATGTTTCTTACATCAA | GCGGATACCGTTTATAAGCTGAACGGTAAAGCGGAGAT |
| | TCAATTGGTAGATGACGGGAGAATTTTTCTATTTCA | CTTCTTTCGTGAGAAGAGCCTGAACTACAGCAAGGAGA |
| | AATTTATAACAAAGACTTTTCTCCATACAGCAAGG | TTATGGAAAAAGGCCACCACCGTGATGAACTGAAAGA |
| | GCAAACCCAATTTGCATACCATGTATTGGAGAGCT | CAAGTTCAGCTATCCGATCATTAAAGACAAGCGTTTTG |
| | GTTTTCGATGAAAAAAACTTAGCAGATACGGTATA | CGCTGGATAAGTTTCAGTTCCACGTTCCGCTGACCATG |
| | TAAACTGAACGGAAAAGCCGAGATATTTTTTAGAG | AACTTTAAAGCGGGTAGCAACCCGAACCTGAACGATCG |
| | AAAAGTCGCTCAACTACTCTAAAGAAATCATGGAA | TGCGCTGGACTTCCTGAAGGATAACCCGGACATCAAAA |
| | AAAGGGCATCATCGAGACGAATTGAAGGATAAATT | TCATTGGTCTGGATCGTGGCGAGCGTCACCTGCTGTAC |
| | TTCTTACCCTATTATCAAGGATAAACGATTTGCCTT | CTGAGCCTGATCGACCAGAAAGGCAACATCATTGAGCA |
| | GGATAAGTTTCAGTTTCATGTCCCATTAACAATGAA | ATATACCCTGAACGAAATTGTGAGCAAACACAAGGAC |
| | CTTTAAGGCGGGAAGCAATCCAAATTTAAACGACC | AAAACCTTTAAAAAGGATTACCACGAGCTGCTGGACAA |
| | GTGCATTGGATTTCTTAAAAGATAATCCCGATATA | AAAGGAAAAGGGTCGTGACGATGCGCGTAAAAACTGG |
| | AAAATCATTGGCTTGGACAGAGGAGAGCGACACCT | GACGTTATCGAAACCATTAAGGAGCTGAAAGAAGGCT |
| | ACTCTACTTGAGCCTGATTGATCAAAAAGGAAATA | ATCTGAGCCAGGTGGTTCACAAGATTGCGCAAATGATG |
| | TAATTGAGCAATACACATTGAATGAGATTGTTTCA | ATCGAGCACAACAGCATTGTGGTTCTGGAAGATCTGAA |
| | AAACACAAAGACAAAACCTTTAAAAAAGACTATC | CGCGGGTTTCAAACGTGGCCGTCATAAGGTGGAGAAGC |
| | ACGAGCTATTAGATAAGAAAGAAAAGGGGCGTGA | AGGTTTACCAAAAGTTCGAAAAGATGCTGATCGACAAG |
| | TGATGCTCGAAAAAATTGGGATGTTATCGAAACGA | CTGAACTATCTGGTGTTCAAAGACCACGATAAGGAGAA |
| | TTAAGGAATTAAAAGAGGGATACCTTTCTCAGGTA | ACCGGGTGGCCTGCTGAACGCGCTGCAGCTGACCAACA |
| | GTTCACAAAATTGCTCAAATGATGATTGAGCACAA | AGTTCGAGAGCTTCCAGAAGCTGGGTAAACAAAGCGG |
| | CTCAATTGTTGTATTAGAGGATTTAAACGCTGGCTT | CCTGCTGTTCTACGTTCCGGCGGCGCTGACCAGCAAAA |
| | TAAAAGAGGAAGGCATAAGGTAGAAAAGCAAGTT | TCGATCCGGCGACCGGTTTCACCAACTTTCTGCGTCCGA |
| | TATCAGAAGTTTGAGAAAATGCTCATTGATAAATT | AGCACGAGAGCATTCCGAAAAGCCAGAGCTTCATCGCG |
| | GAATTATTTGGTTTTTAAAGACCATGATAAGGAAA | GGCTTTACCCGTATTCACTTTAACAGCGAGAAGGAATA |
| | AACCTGGAGGTTTACTGAACGCTCTTCAACTCACA | CTTTGAGTTCAAGTTTGACCTGAAAAACATCCCGAACA |
| | AATAAATTCGAAAGTTTTCAAAAATTAGGTAAACA | CCCGTTTCCCGGACGATACCAAGACCGAATGGACCGTG |
| | AAGCGGTCTTCTTTTTTATGTACCTGCTGCTTTAAC | TGCACCACCAACGTTCCGCGTTATTGGTGGAACAAAAG |
| | AAGTAAAATTGATCCTGCTACAGGTTTTACGAATTT | CCTGAACGAGGGCAAGGGTGGCCAGGAAAAAGTGCTG |
| | CTTAAGACCAAAGCATGAAAGCATCCCCAAATCCC | GTTACCCAGCGTCTGCAAGATCTGCTGGCGCGTTATGA |
| | AATCTTTCATCGCAGGCTTTACCCGAATTCATTTTA | CCTGGGTTACGCGACCGGCGAGAACCTGAAAGAGGAC |
| | ATTCGGAGAAAGAATATTTCGAGTTTAAATTCGAT | ATCCTGACCATTGAGGACGCGAGCTTCTACAAAGAATT |
| | TTGAAAAACATACCGAATACACGCTTTCCTGATGA | TCTGTGGCTGCTGAACGTGACCGTTAGCCTGCGTCACA |
| | TACAAAAACTGAATGGACGGTATGTACAACAAATG | ACAACGGCAAGCACGGCGAGCTGGAGGAAGATGCGAT |
| | TGCCTCGTTATTGGTGGAACAAGAGTTTGAATGAA | CATTAGCCCGGTGGCGAACGCGCAGGGCGAGTTCTTTA |
| | GGTAAAGGGGGACAAGAAAAGGTCTTAGTAACAC | ACAGCAGCGAAGCGAAGAGCAGCGCGCCGAAAGACGC |
| | AAAGGCTGCAAGATTTATTGGCAAGGTATGATTTA | GGATGCGAACGGTGCGTACCACATCGCGCTGAAAGGCC |
| | GGCTATGCAACTGGTGAAAACTTAAAGGAAGATAT | TGTGGGCGCTGCGTACCATTAACGCGCACGACAAAAAG |
| | TTTAACAATTGAAGATGCCTCTTTCTACAAGGAGTT | GAGTGGCGTGGCATCAAGCTGGCGATTAGCAACAAAG |
| | CTTATGGTTGTTGAATGTAACTGTTTCATTGCGGCA | AATGGCTGCAATTCGTTCAGCAAAAGCCGTTTCTGAAA |
| | CAATAATGGTAAGCATGGAGAACTAGAAGAAGAT | CCGTAG (SEQ ID NO: 26) |
| | GCGATCATTTCACCCGTAGCGAATGCACAAGGCGA | |
| | ATTTTTCAATTCGAGTGAGGCAAAGTCTTCAGCCCC | |
| | TAAAGATGCTGATGCCAATGGAGCTTATCATATTG | |
| | CACTTAAAGGACTTTGGGCTTTACGAACAATTAAT | |
| | GCACACGACAAGAAAGAATGGAGAGGTATAAAGT | |
| | TAGCCATATCTAACAAAGAATGGTTGCAGTTTGTG | |
| | CAGCAAAAGCCTTTTCTTAAACCATAG (SEQ ID | |
| | NO: 25) | |
| Type V | ATGAAACAAGAAAAGAAGACAGAAAAATCCGTGT | ATGAAGCAGGAGAAGAAAACCGAGAAGAGCGTGTTCA |
| Cas_4 | TCTCGGATTTTACAAATAAATACGCACTTTCGAAG | GCGATTTCACCAACAAGTACGCGCTGAGCAAAACCCTG |
| | ACGTTGCGATTTGAGTTGAAGCCGGTGGGAGAGAC | CGTTTCGAGCTGAAGCCGGTGGGTGAAACCCTGGAGAA |
| | GCTTGAAAATATGAAAGATGCTTTTGGATATGACA | CATGAAAGACGCGTTTGGCTACGATAAGAAAATGCAG |
| | AAAAAATGCAAACTTTTTTGAAAGATCAAGAAATC | ACCTTCCTGAAGGACCAAGAGATCGAAGATGCGTATCA |
| | GAAGATGCGTATCAAAACCTCAAGCCCATTCTCGA | GAACCTGAAACCGATTCTGGACCGTATCCACGAGGAAT |
| | TAGAATTCACGAAGAATTCATTACACAAAGCCTTG | TTATTACCCAAAGCCTGGAGAGCGAACAGGCGAAGCA |
| | AATCAGAACAAGCAAAACAAATTCCATTTCATATA | AATTCCGTTCCACATCTACGAGAAAAGCTATCGTAAGA |
| | TATGAAAAATCTTATAGAAAAAAGAGCGAAATTAC | AAAGCGAAATCACCCTGAAGCAGTTTGAAACCGTGGA |
| | ACTCAAGCAGTTTGAAACGGTTGAGAAAAAAATAC | AAAGAAAATTCGTGAGTACTTCGATGAAGCGTATAAAC |
| | GAGAGTATTTTGACGAAGCGTATAAACAAACAGCT | AGACCGCGCAAGTTTGGAAGCAAAACGCGCCGAAAGA |
| | CAAGTGTGGAAGCAGAATGCTCCAAAAGACAAAA | TAAGAAAGGTAAGGGCGTGTTCACCAAGGACAGCCAC |
| | AAGGGAAGGGGGTATTTACAAAAGATTCTCACAAG | AAACTGCTGACCGAGGTGGGTGTTCTGGAATACATCCG |
| | CTCCTTACTGAGGTGGGAGTGCTTGAATATATTCGT | TCAGAACACCGAGAAGTTTAGCGACATTCTGCCGAAAA |
| | CAAAATACGGAGAAATTTTCAGACATTCTTCCGAA | GCGAGATCGAACAACACCTGAACGTTTTCAGCGGTTTC |
| | AAGTGAAATAGAGCAACATCTCAATGTTTTTAGTG | TTTACCTATTTTCAGGGCTTCAGCCAAAACCGTGAGAA |
| | GATTTTTTACCTATTTCCAAGGATTTAGTCAAAATA | CTACTATACCACCAAGGATGAAAAAGCGACCGCGGTG |
| | GAGAAAATTACTATACAACAAAGGATGAAAAAGC | GCGACCCGTGTGGTTAGCGAGAACCTGCCGAAGTTTTG |
| | AACGGCGGTAGCAACAAGAGTTGTCAGTGAAAATC | CGACAACATCCTGACCTTCGAGAACAAGAAAGAAGCG |
| | TTCCGAAATTTTGTGACAACATCCTAACCTTTGAGA | TACCTGGCGCTGTATCAGAGCCTGGCGGAAAAGGGTAA |
| | ACAAAAAAGAAGCGTACCTCGCTCTGTATCAATCT | AACCCTGCAAATTAAAGATGGTAGCAGCGGCAAGATG |
| | TTGGCTGAGAAGGGGAAAACACTTCAGATAAAAG | AAAAGCCTGGAGGGCGTTGACGAAGCGATGTTTAGCAT |
| | ATGGGTCATCAGGAAAAATGAAATCTCTTGAAGGG | CCACCACTTCAACGAGTGCCTGAGCCAGCGTGAGATTG |
| | GTGGATGAAGCAATGTTTTCAATACATCATTTCAAT | AAAAGTACAACGAAGCGATCGCGAACGCGAACTACCT |
| | GAATGTCTTTCACAAAGAGAGATTGAGAAATATAA | GATTAACCTGTATAACCAGCTGCAAGACGATAAGAAAA |
| | TGAGGCAATAGCCAATGCTAATTATCTTATAAACC | ACAAGCTGAAACTGTTCAAGACCCTGTACAAACAAATT |
| | TCTATAATCAATTACAAGATGACAAGAAGAATAAA | GGTTGCGGCGACAAGGAAACCTTCATCGAAAAAATTAC |
| | CTTAAGCTTTTCAAAACTCTCTACAAACAAATAGG | CCACTATACCGAGGAAGAGGCGCAGAAGGCGCGTAAA |
| | GTGTGGGGATAAGGAAACGTTTATCGAGAAGATAA | GAGAAGAAAGAAAAAGCGATCAGCCTGGAGCAAGAAC |
| | CTCACTACACAGAAGAAGAGGCACAAAAAGCTCG | TGAAGGAGTTCAGCAGCCTGGGTAGCAAATACTTCTTT |
| | AAAAGAAAAAAAGGAAAAAGCAATATCACTTGAA | GGCATTAGCGAGAACGAATTTATCCGTACCGTTGAGGA |
| | CAGGAATTAAAAGAGTTTTCTAGTTTGGGAAGTAA | TTTCCGTAAGTATCTGCTGGAAGAGAAAGAAGACTACG |
| | ATATTTTTTCGGTATATCAGAAAATGAGTTTATTAG | CGGGTGTGTATTGGAGCAAGCAGGCGATCAACAACATT |
| | AACAGTAGAAGATTTCAGAAAGTATCTCTTAGAAG | AGCGGCAAATACTTTAGCAACTGGCACGCGCTGAAGGA |
| | AAAAAGAAGATTATGCGGGAGTCTATTGGTCAAAA | CATCCTGAAAGAGAAGAAAGTTTTCAGCACCAGCGCGA |
| | CAGGCGATAAACAATATATCGGGGAAATATTTTTC | GCAAGGACGAAAGCGTGAGCATCCCGGAGATCATTGA |
| | TAATTGGCATGCACTTAAAGATATTCTCAAAGAAA | ACTGAAGCAACTGTTTGAAGTTCTGGACGGTATTGAGA |
| | AAAAGGTTTTTAGCACGAGCGCTTCCAAAGATGAA | AATGGGAAGTGCCGGATAACTTCTTTAAGAAAACCCTG |
| | TCGGTGAGCATCCCGGAGATAATTGAACTCAAGCA | ACCGAAGAGGTTAGCAAGGACCACCGTGATTTCCAGAA |
| | ACTTTTTGAGGTTCTTGATGGAATTGAGAAGTGGG | AAACGCGAAGCGTAAAGAGATCATTAAGAGCAGCCAA |
| | AAGTACCTGATAATTTTTTCAAAAAGACGCTTACA | AAACCGAGCGAAGCGCTGCTGCGTATGATGTTTGACGA |
| | GAGGAGGTAAGTAAAGATCATAGAGATTTCCAGAA | TATGGTGGATCTGCGTGAGAAATTCCTGAGCAAGAAAG |
| | AAATGCAAAAAGAAAAGAGATCATTAAATCATCCC | AGGACATCCTGGAAAACACCAACTACACCACCCAGGA |
| | AAAAACCATCAGAAGCACTTCTGAGGATGATGTTT | GCGTAAGGACGACATCAAAGAATGGATGGACAGCGGT |
| | GATGATATGGTTGATCTTCGAGAGAAATTTCTTTCC | CTGCGTATCATTCAGATTCTGAAGTACTTCAGCGTGCA |
| | AAAAAAGAAGACATTTTGGAAAATACAAACTATAC | AGAAAAGAAAATCAAGGGCACCCCGTTCGACGCGAAG |
| | TACTCAAGAAAGAAAGGATGATATAAAAGAATGG | ATTAAAGAGGGCCTGGATACCCTGCTGCTGAGCAACGA |
| | ATGGATTCGGGATTGAGAATTATTCAAATTCTCAA | AGTTGACTGGTTTACCCGTTACGATCGTGTGCGTAGCTT |
| | ATACTTTTCTGTCCAAGAAAAGAAGATAAAAGGGA | CCTGACCAAGAAACCGCAGGACGATGCGAAGGAGAAC |
| | CACCATTTGACGCCAAAATCAAAGAAGGGCTTGAC | AAGCTGAAACTGAACTTTGAAAACAGCACCCTGGCGGG |
| | ACTCTCCTTCTCTCCAATGAAGTGGACTGGTTTACA | TGGCTGGGACGTTAACAAAGAGAGCGATAACAGCTGC |
| | AGATATGATCGCGTACGAAGTTTTCTCACTAAAAA | ATCATTCTGAAGGAAGAGGAAAAAACCTTCCTGGCGGT |
| | ACCGCAAGATGATGCGAAAGAAAATAAATTGAAG | GATTGCGAAGAGCAAAGGCAAGGAGAAAAACAACGCG |
| | TTGAATTTTGAGAATAGCACGCTTGCTGGTGGGTG | CTGTTTCGTAAGACCGAACAAAACCCGCTGTTCAGCAT |
| | GGATGTGAACAAAGAAAGTGATAACTCTTGCATCA | CGAGAACGCGGAAACCATGAAGAAAATGGAGTACAAG |
| | TTTTGAAAGAGGAAGAAAAAACATTCTTAGCCGTG | CTGCTGCCGGGCCCGAACAAGATGCTGCCGAAATGCCT |
| | ATAGCAAAATCAAAAGGGAAAGAGAAAAATAATG | GTTTCCGAAAAGCAACCCGAAGAAATACGGTGCGACC |
| | CTTTGTTTCGAAAAACAGAACAAAATCCACTTTTTT | GAAACCGTGCTGGACGTTTATAAGAAAGGCAGCTTTAA |
| | CTATTGAGAATGCGGAGACAATGAAAAAAATGGA | GAAAAACGAGGAAAACTTCAGCAAGAAAGACCTGTAC |
| | GTATAAGCTTCTCCCCGGTCCAAATAAAATGTTGC | ACCGTTATCGATTTCTATAAAGAGGCGCTGAAACGTTA |
| | CGAAGTGTCTTTTTCCCAAGTCGAATCCTAAGAAA | CGAAGGTTGGAACTGCTTCGAGTTTCACTTCAAGAAAA |
| | TATGGAGCAACTGAAACTGTTCTTGATGTGTATAA | CCAGCGAATACAACGACATCGGCGAGTTTTATCTGGAT |
| | AAAAGGAAGTTTTAAGAAGAACGAAGAAAATTTCT | GTTGAAAAGAAAGGCTATACCCTGGACTTCGTGGATAT |
| | CCAAAAAAGATTTATACACTGTAATTGATTTTTACA | TAACCGTAACGTGCTGGGCCAGTACGTTGAGGATGGCC |
| | AGGAGGCTTTGAAGAGATATGAAGGATGGAATTGT | GTGTGTACCTGTTCGAAATCCGTAACAAAGACTGGAAC |
| | TTTGAATTTCATTTTAAAAAGACGAGTGAATACAA | ACCCTGCCGGATGGTAGCAAGAAAAGCGGCAACACCA |
| | TGATATTGGTGAATTTTATTTAGATGTTGAAAAGAA | ACCTGCACACCATGTACTGGAAGGCGCTGTTTCAAGAC |
| | AGGATACACTTTGGATTTTGTAGATATTAACAGAA | CGTGAGAACCGTCCGAAACTGAACGGCGAGGCGGAAA |
| | ATGTCCTTGGACAGTATGTTGAAGATGGAAGGGTG | TCTTCTATCGTAAGGCGCTGAGCAAGGACGAAATTAAG |
| | TATCTTTTCGAAATTCGAAATAAAGACTGGAATAC | AAAAAGAAAGAT |
| | ACTACCTGATGGATCGAAGAAAAGCGGAAATACA | AAGCACGAGAAAGAAGTTATCGAGAACTACCGTTTTAG |
| | AATCTCCATACTATGTACTGGAAAGCATTGTTTCAA | CAAGGAAAAATTTCTGTTCCACGTGCCGATTACCCTGA |
| | GATAGAGAAAATCGACCAAAACTCAATGGAGAGG | ACTTCTGCCTGAAG |
| | CTGAGATTTTTTATAGAAAAGCCTTATCAAAAGAT | GATTATAAAATTAACGACGACATCAACGAGAAGCTGCT |
| | GAAATAAAGAAGAAAAAAGATAAACATGAAAAGG | GGAGAACGAAAACGTTTGCTTCCTGGGTATTGACCGTG |
| | AAGTTATTGAAAATTATCGATTTTCCAAAGAAAAA | GCGAAAAACACCTG |
| | TTTCTTTTTCATGTGCCAATAACGCTCAACTTTTGTC | GCGTACTATAGCATCGTGGACAACGAGGGTAACATTCT |
| | TCAAGGATTATAAAATCAACGACGATATAAACGAA | GGAACAGGATACCCTGAACACCATCAACGGCAAGGAC |
| | AAGCTCCTTGAAAATGAGAATGTATGCTTTTTGGG | TACAACACCCTGCTG |
| | GATTGATAGGGGAGAAAAGCACCTTGCCTATTATT | GAGGAACGTAGCGAGGAAATGGATACCGCGCGTAAAA |
| | CGATAGTTGATAACGAGGGAAATATTTTGGAACAA | GCTGGCAGACCATCGGCACCATTAAGGAGCTGAAAGAT |
| | GATACACTCAATACGATAAACGGAAAAGACTACA | GGCTACATCAGCCAA |
| | ATACTCTTCTCGAAGAACGATCCGAAGAGATGGAT | GTTATCCGTAAGATTGTGGACCTGAGCCTGCGTTATAA |
| | ACCGCTCGAAAAAGTTGGCAGACTATTGGAACGAT | CGCGTTTATCGTTCTGGAAGACCTGAACGTGGGTTTCA |
| | TAAAGAACTCAAAGACGGCTATATTTCTCAAGTTA | AGCAGGGCCGTCAA |
| | TCCGAAAAATTGTCGATCTCTCTCTTCGATACAATG | AAGATTGAGAAAAGCGTTTACCAGAAACTGGAACTGG |
| | CATTTATTGTCTTAGAAGATCTCAATGTTGGGTTCA | CGCTGGCGAAGAAACTGAACTTCCTGGTGGAGAAGAG |
| | AACAAGGTCGCCAAAAAATCGAAAAATCCGTTTAC | CGCGCACCAGGGTGAA |
| | CAAAAACTCGAGCTTGCTTTGGCGAAAAAACTCAA | ATGGGCAGCGTTACCAAAGCGCTGCAACTGACCCCGCC |
| | TTTTCTTGTGGAGAAATCTGCCCATCAAGGAGAGA | GGTGAACACCTTTGGTGATATGGAGAAGCGTAAACAGT |
| | TGGGATCTGTCACAAAAGCACTTCAGCTCACACCA | TCGGCATCATGCTG |
| | CCGGTAAATACCTTCGGAGATATGGAAAAACGAAA | TACACCCGTGCGAACTATACCAGCCAAACCGACCCGGC |
| | ACAATTTGGTATTATGCTTTACACCAGAGCGAACT | GACCGGTTGGCGTAAAACCATCTACCTGAAGCGTGGTG |
| | ATACATCCCAAACCGACCCTGCTACAGGATGGCGA | GCGAGAAACTGATT |
| | AAAACAATATATCTCAAACGAGGAGGTGAAAAAC | CGTGAAAACATCATTCAGAGCTTTGACGATATGTATTT |
| | TCATACGAGAAAATATTATCCAGTCCTTTGATGATA | CGACGGCAAGGATTACGTTTTTAGCTATACCGAAAAAT |
| | TGTACTTTGATGGAAAAGATTATGTCTTTTCGTATA | TCGGCAAGGATAAAAACAACCAACGTAGCGGCCGTAG |
| | CCGAAAAATTCGGAAAAGACAAAAACAATCAGAG | CTGGAAGCTGTACAGCGGTAAAGACGGCATTAGCCTGG |
| | AAGTGGAAGAAGTTGGAAGCTCTACTCAGGAAAA | ATCGTTTTCGTGGCAAGCGTGGCAAAGAGTTCAACGAA |
| | GACGGCATCTCCCTTGATCGGTTTCGAGGAAAGCG | TGGAGCGTGGAAACCATCGACATTGCGGGTATCCTGAA |
| | AGGAAAAGAATTTAATGAATGGAGCGTTGAGACG | CGAGCTGTTTGAAGACTTCGATAAGAACATTAGCCTGC |
| | ATTGATATAGCGGGGATACTTAATGAATTATTTGA | TGGAACAGATCCAGCAAGGCAAAGATCCGAAGAAAAT |
| | AGATTTTGACAAAAATATTTCTCTCTTGGAACAAAT | CAACGAGCACACCGCGTATGAAACCCTGCGTTTTGTTA |
| | ACAACAAGGCAAAGATCCAAAGAAGATAAACGAA | TCGACAGCATTCAGCAAATCCGTAACAGCGGCGAGAA |
| | CACACCGCATATGAAACATTGCGGTTTGTAATTGA | GGGCGACGAACGTAACAGCGATTTCCTGCACAGCCCGG |
| | TTCAATACAGCAAATACGAAACTCGGGAGAAAAA | TTCGTAACACCGAGGGTGAACACTACGACAGCCGTATT |
| | GGTGATGAAAGAAATAGTGATTTTCTTCACTCACCT | TATCTGGATCGTGAGAAGGAAGGCATTGTGACCGACCT |
| | GTGAGAAATACAGAAGGTGAGCATTATGACTCGAG | GCCGATCAGCGGTGATGCGAACGGCGCGTACAACATTG |
| | AATCTATCTTGATCGAGAAAAAGAGGGAATAGTTA | CGCGTAAAGGTATCCTGATGAAGGAGCACCTGAAACGT |
| | CAGATCTTCCCATCTCAGGAGATGCCAATGGTGCG | GACCTGAGCGAATATATCAGCGATGAGGAATGGAGCG |
| | TACAATATCGCTCGAAAAGGAATTCTTATGAAAGA | TGTGGCTGAGCGGCAAGAACCGTTGGGAGAAATGGAT |
| | GCACCTCAAGAGAGATCTATCTGAATACATATCCG | GCAGGAGAACGAAAAAGACCTGCGTAAGAAAAAGAAA |
| | ATGAAGAATGGTCTGTATGGCTTTCGGGAAAAAAT | TAG (SEQ ID NO: 28) |
| | AGATGGGAGAAATGGATGCAAGAAAATGAAAAAG | |
| | ATTTAAGAAAGAAGAAAAAATAG (SEQ ID NO: | |
| | 27) | |
| Type V | ATGAAAAATAACAGAACAAAACACTTACACCCAA | ATGAAAAACAACCGTACCAAGCACCTGCACCCGACCG |
| Cas_5 | CAGGGTATCAACTAGCAAGCGAGCGTATCAAGCAA | GTTACCAGCTGGCGAGCGAGCGTATTAAACAAGCGCC |
| | GCTCCATTAAACAAAAACTCAAAATACATAGTAAC | CTGAACAAGAACAGCAAATACATCGTGACCGTTAAGTA |
| | AGTTAAGTATCCTCTCAAAGGAGATCTCAAGGGAA | TCCGCTGAAAGGTGATCTGAAGGGCAAACTGGAGAGC |
| | AACTTGAGTCCGAGTTAATAGAGCAATCCTTCCGG | GAACTGATTGAACAGAGCTTCCGTGACTACGCGTATGC |
| | GATTATGCATACGCGTATGGAATTCCCACGCTAAA | GTACGGTATCCCGACCCTGAAGGAGAGCAAACCGCAA |
| | GGAATCAAAACCTCAGGTTTCACTTATTGATTTTTA | GTGAGCCTGATCGACTTYTACATTGAATGCCTGCGTATG |
| | TATTGAGTGTTTGCGTATGGGGGCATTTTTTCAACC | GGCGCGTTCTTTCAGCCGAGCAGCGCGAAACTGCAAGA |
| | CTCATCAGCCAAGCTTCAAGATTTGGCTTCGGGTG | TCTGGCGAGCGGTGGCAAGCTGCAGGCGCTGATCAAGA |
| | GGAAGCTTCAAGCACTTATAAAGAAAAACATTCCA | AAAACATTCCGGACCACATCCTGGTGAAACTGAACATG |
| | GATCACATCCTCGTGAAACTTAACATGCTTGAGTTT | CTGGAGTTCGTTGACGGTATTACCGCGGATTTTCGTAA |
| | GTAGATGGTATCACCGCTGACTTTCGCAAAATGGA | GATGGAACAAGAGGAACCGGCGACCTTCCGTAAGAAA |
| | GCAGGAAGAGCCTGCAACATTTCGAAAAAAAATA | ATCGCGAAGTGGTTTAAAGACGATACCGACCCGTACAT |
| | GCTAAATGGTTCAAGGATGATACAGATCCCTATAT | TGATCAGGTGGTTGAGATCTATCTGCAGAACGGCCAAA |
| | TGATCAGGTTGTGGAGATTTATTTGCAGAACGGCC | GCCAGCAAACCCAAAGCGCGGAAAGCGCGTTCTTTTAC |
| | AATCTCAGCAAACACAATCTGCTGAATCGGCTTTTT | CGTCCGAAGAAAAACCCGAGCAACCTGACCTTCTATCT |
| | TCTATCGTCCAAAGAAGAATCCTTCCAATCTAACTT | GCACCCGGAAATTCTGGTGGACCCGAGCGAGAGCAAC |
| | TTTATTTACATCCAGAAATTCTAGTGGACCCTTCGGC | CGCAAAAAGTGGTTTTTGAGAGCGTTCGTCAGATCTA |
| | AGAGTAATCCCCAAAAAGTTGTGTTTGAAAGCGTG | CACCGCGCTGAACAACCAGCTGCAACCGCCGGAAAAG |
| | AGACAAATTTATACTGCCTTAAATAATCAGCTTCA | AAACGTGAGGACTTCGATCTGGAACTGATCGGTCTGGA |
| | GCCGCCTGAAAAAAAGAGAGAAGATTTTGATCTTG | TAAACAGGCGAACGCGCTGAGCAACTTCTTTAACAACG |
| | AATTAATAGGATTAGATAAACAAGCGAACGCTTTA | TGTTTAACCGTCTGCAGAAGGACGATGTTCAAAGCCTG |
| | ATCGAACTTTTTTAACAATGTGTTTAATCGGTTGCAA | TGGCGGAAATTCTGGACCTGAGCGAGCTGTGGCGTGG |
| | AAAGATGATGTGCAATCCCTTATGGCCGAGATCCT | CAAGGAGCAGGAACTGGAGCAACGTCTGATCCACCTG |
| | TGATCTCTCCGAACTTTGGAGAGGGAAAGAGCAAG | AGCAGCGTGGCGAAACAGGTTGGTAACCCGGCGCTGG |
| | AGCTTGAACAAAGACTGATCCACTTATCTAGTGTT | GCAAGAGCTGGGCGGATTACCGTGCGATGTTCAGCGGC |
| | GCAAAACAGGTTGGAAATCCAGCGCTGGGAAAAA | CGTATCAAGAGCTGGTATAAAAACACCGTGAACCACCT |
| | GTTGGGCTGATTACAGGGCTATGTTCTCTGGAAGG | GAAAGCGCGTGAGGAACAGCTGCCGAACCTGAAGGAA |
| | ATAAAATCTTGGTATAAAAACACAGTGAATCATCT | GCGGTTGAGGTGGTTATTGCGGACGTTCGTCAAGTGGT |
| | AAAAGCTAGAGAAGAACAACTACCCAACCTGAAA | TGAGCTGATCACCAACAAGAGCTTCGACGAACGTGATA |
| | GAAGCAGTCGAGGTTGTGATAGCAGATGTCAGACA | ACAGCAACCGTACCGAACTGCTGTTCCACTTTCTGGAG |
| | GGTAGTTGAGTTAATAACAAATAAATCATTTGATG | AGCTGCCAGGCGCTGCTGGACGCGCTGGATCAAAACAA |
| | AAAGAGATAACTCGAATCGGACCGAACTTCTATTT | CGAGGATGTGTGCTTCCAGCTGCACGCGGAACTGACCC |
| | CATTTTTTAGAATCTTGCCAAGCGTTACTTGATGCG | GTGACTTTAACCTGGTTCTGCAGCGTTACGCGCAAGAG |
| | CTTGATCAGAATAATGAAGATGTTTGTTTTCAGCTG | TTCCTGACCCTGGAGAACAGCAAGAAAAAGAAAAAGC |
| | CATGCTGAATTGACTCGTGATTTCAATCTTGTGCTT | AGTTTGCGGAGGATAGCGCGGAAGCGCTGGAGCTGATC |
| | CAGCGGTATGCACAAGAATTCCTCACCCTTGAGAA | CGTCCGAAGTATGCGAAACTGTTCAGCCGTCTGCGTCC |
| | TTCTAAGAAGAAGAAAAAACAGTTTGCTGAAGATT | GCAGCCGGCGTTCTTTGGCGAGCAACGTGCGAAACTGG |
| | CAGCGGAAGCACTAGAGCTTATTCGACCTAAATAC | TTGACCGTTACAGCGAAGCGGCGAAGCAGCTGTTCCAA |
| | GCAAAACTTTTCTCAAGATTACGGCCCCAGCCAGC | CTGCTGACCTTTCTGCAGCAACTGATCCTGGACCTGTAT |
| | ATTTTTTGGTGAGCAACGGGCGAAACTTGTGGATC | GCGCTGCCGCGTGGTGATGCGCTGGGTGAAGAAACCCT |
| | GTTACTCGGAAGCAGCAAAGCAACTATTTCAACTC | GCTGCAGATTGTGGATAAAGTGGTTAAGCGTAAAAACA |
| | TTAACTTTCTTACAACAACTGATTCTTGATCTCTAC | ACGCGAACACCATCAACCACCAGCAACTGTTCAAGGAC |
| | GCCCTGCCTCGTGGTGATGCACTTGGAGAAGAAAC | CTGTTCACCCAAGCGATCATTCGTCCGTACACCAAGGA |
| | ACTTTTGCAAATTGTGGACAAGGTTGTGAAAAGAA | CGAGAAAGTTGCGTATTTCATTAACCCGAACGCGAGCC |
| | AAAATAATGCAAATACAATAAATCATCAGCAACTT | GTCTGCGTCTGCGTAAGCTGGAGAAGAGCTGGCGTCTG |
| | TTTAAAGACCTGTTTACCCAAGCAATCATTCGGCC | CCGGACGTGGAACTGGTTCAGATGATCGAGAGCACCCT |
| | GTATACCAAAGATGAAAAAGTTGCTTATTTTATCA | GCTGAAGAGCTTTAACCTGAGCCAAGAGGCGTACAGCC |
| | ACCCAAATGCTTCTAGATTGAGATTGAGAAAATTA | ACGCGGACAGCGAAAGCCTGATCGATGCGATTGAGAG |
| | GAAAAAAGCTGGAGATTGCCTGATGTTGAGTTGGT | CAGCAAAACCCTGGTGGCGGTTCTGCTGCTGACCCGTA |
| | CCAAATGATTGAAAGCACCTTGCTTAAGTCCTTCA | AGAGCACCCAGTATAGCTTCGATTTTGAAAAAATTCCG |
| | ATCTATCGCAAGAAGCGTACTCACATGCTGACTCA | AGCGAAACCCTGCGTTTCAAGATCAACCGTCTGGACAA |
| | GAATCACTTATCGATGCTATTGAATCCTCAAAAAC | AAAGAACCGTGTGCAGTACCTGCAACGTGCGACCAGCT |
| | ACTCGTTGCGGTTTTATTATTGACTCGAAAAAGTAC | TTATTGGCACCGAGCTGCGTGGCTATATCAGCCTGATT |
| | CCAATATTCTTTTATTTTGAAAAGATTCCGTCCGA | AGCCGTAGCGAAGTGATCGACCGTGCGACCGTTCAGCT |
| | GACGCTTCGATTCAAGATCAACCGCCTAGATAAGA | GAGCAACAGCGATAAAATGTTCACCCCGGTGCGTACCA |
| | AGAATAGAGTTCAATATCTTCAGCGAGCGACTTCA | AAGACAACCGTTGGAAGATTGCGCTGAACCACGAAAA |
| | TTCATTGGGACAGAGTTGAGAGGGTATATTTCTCTT | GGCGGCGATCGGTCTGGATCAGGAAGTTGAGAAGTTCA |
| | ATTTCTCGATCCGAAGTTATTGATCGAGCAACAGT | CCAAAAGCGGCGTGAAACGTGAGGTTCTGAAGCACCA |
| | GCAACTGAGTAATTCCGATAAGATGTTTACTCCTGT | AACCCTGGACATCAAAACCAGCCGTTACCAGCTGCAAT |
| | TCGAACGAAAGACAATAGATGGAAAATAGCATTG | TTCTGGAATGGCTGCACAAGACCCCGAAAAAGAAACA |
| | AATCACGAAAAAGCAGCAATAGGACTAGATCAAG | GCACCTGAACATTGCGCTGAACGAACCGAGCCTGATTG |
| | AGGTTGAAAAATTTACAAAGTCGGGGGTAAAGAG | CGGAGAAGAAATACCGTATCAACTGGACCGTGCAGAA |
| | AGAGGTGCTTAAACATCAAACCTTAGATATCAAGA | CCAAATCCTGGTGCCGGAATATGTTCTGCTGGAGAGCG |
| | CCTCAAGATACCAACTTCAGTTTCTAGAATGGTTGC | GTGTTTTCCTGAGCATTCCGTTTACCATCAGCCCGGCGA |
| | ACAAAACTCCAAAAAAGAAACAGCATCTCAATATC | AGGATAACAACAAGAGCTTCAGCCGTTACCTGGGCCTG |
| | GCATTGAATGAACCCTCACTTATTGCTGAGAAAAA | GACCTGGGCGAGTTTGGCGTGGCGTGGGCGGTTCTGGG |
| | ATATCGAATCAATTGGACTGTGCAAAATCAAATTT | TATTAAAGATAACCGTCCGTATCTGGTTCAGACCGGCA |
| | TAGTCCCAGAATATGTTTTGCTTGAATCTGGGGTAT | TGCTGCAGGACCCGCAACTGCGTGCGATCGCGAACGAA |
| | TTCTTTCAATACCTTTTACGATTAGTCCAGCGAAAG | GTGGCGGTTATGAAGGCGCGTCAAGTTACCGGCACCTT |
| | ATAATAATAAAAGCTTCTCTCGTTATTTGGGACTAG | TGGCGTTCCGAGCAGCCGTCTGCAGCGTCTGCGTGAGA |
| | ACTTAGGGGAATTTGGTGTTGCTTGGGCAGTTCTTG | GCGCGGTGCACAGCCTGGTTAACCAAATTCACAGCCTG |
| | GGATTAAAGATAACAGGCCGTATTTAGTGCAGACG | GTGCTGCGTTACGGTGCGAAAATGGTGTTCGAACGTCA |
| | GGCATGCTTCAAGATCCTCAATTACGAGCAATTGC | GGTTGACGCGTTTCAAACCGGCAGCAACCGTGTTAAGA |
| | TAATGAAGTAGCTGTCATGAAGGCGAGACAAGTAA | AAATCTATGCGAGCCTGAAGCAGGGTAACATTTTCGGC |
| | CCGGAACTTTTGGCGTTCCAAGCTCTCGCCTTCAAA | CGTAAGGAGATCGATAAAAGCAACTATAAGCGTTACTG |
| | GACTTCGGGAAAGCGCAGTGCATTCGTTAGTGAAT | GAGCTATCGTGACGGTCACTTTATGGGCAGCGAGGTGA |
| | CAAATTCATTCTTTGGTGTTGCGGTATGGAGCAAA | GCAGCTGGGGCACCAGCTACTTCTGCCCGCACTGCCGT |
| | AATGGTGTTTGAACGACAGGTTGATGCCTTTCAAA | GAATTTCTGCACGACCTGCCGAAGGAAAAAGATGCGTA |
| | CAGGTTCAAATCGAGTGAAAAAAATATATGCTTCA | TGAGCTGGTTAAAGATAGCCCGGAGGAACTGACCCGTC |
| | TTGAAGCAGGGGAATATATTTGGGCGCAAAGAGAT | TGCGTGTGTACAGCGTTAAACAGACCGGTGAAAAGTAC |
| | AGATAAATCAAACTATAAAAGATATTGGAGTTATC | TATGGTTATGTGGAGGGCAACAGCAGCCCGAAGGAAC |
| | GAGACGGTCATTTTATGGGCAGCGAAGTAAGTTCC | AAGTTCTGGCGTTTGCGCGTCCGCCGTACCAGAGCGAT |
| | TGGGGCACAAGTTATTTTTGTCCACATTGTAGAGA | GCGCTGCTGCTGCTGAGCAAGCAAGGCAAAAACCTGA |
| | GTTTCTTCATGATCTTCCAAAAGAGAAGGATGCGT | ACCTGAGCCAGAGCCTGAAAACCGAGCGTGGTGGCCA |
| | ATGAGCTAGTGAAAGATTCCCCAGAAGAATTGACT | GGCGGTGTTCGTTTGCCCGAAGTTTAGCTGCCTGCGTAC |
| | AGGCTTCGAGTATATTCGGTGAAACAAACAGGAGA | CTACGACGCGGATAAACAAGCGGCGGTGAACATTGCG |
| | AAAATATTATGGATATGTTGAAGGAAATAGCAGTC | ATGCGTAAGTGGGCGGAAGATGTTTTCATCGCGACCAA |
| | CAAAAGAACAAGTTCTTGCATTTGCTCGCCCACCA | GGGTAAACCGCCGAAACAGCGTGACGAGAACTATTTCC |
| | TATCAAAGTGACGCGTTACTTTTGTTATCAAAACAG | GTATGCGTAAGGACTTTGAGCGTAAGCTGTACAAAGAT |
| | GGTAAAAATCTCAACTTATCACAAAGTTTGAAAAC | CTGAACGAGTATCCGACCGTTAAGATGGGCGAATAG |
| | CGAACGCGGTGGTCAAGCGGTCTTTGTATGCCCCA | (SEQ ID NO: 30) |
| | AATTTTCATGTTTGAGGACTTATGATGCTGATAAGC | |
| | AAGCAGCGGTAAATATTGCGATGCGCAAATGGGCT | |
| | GAAGACGTATTTATTGCTACTAAAGGTAAGCCTCC | |
| | AAAGCAAAGGGATGAGAATTATTTTAGAATGAGGA | |
| | AAGATTTTGAAAGAAAATTATATAAAGATTTGAAT | |
| | GAATACCCAACCGTTAAAATGGGTGAGTAG (SEQ | |
| | ID NO: 29) | |
| Type V | ATGGCGCGTAAGGACAAATACCGCGGGCTGACCG | ATGGCGCGTAAGGACAAATACCGTGGTCTGACCGGCTA |
| Cas_6 | GCTACCGTCTGCACCAGAAGCGGCTGGAGCGCTCG | TCGTCTGCACCAAAAGCGTCTGGAACGTAGCGGTAAAC |
| | GGTAAGCAGGGTATTCGCACCATTAAGTATCCGCT | AGGGCATCCGTACCATTAAGTACCCGCTGGTTGGTGCG |
| | CGTTGGCGCGACGGAGGAGCACCATGAGCAATTCG | ACCGAGGAACACCACGAGCAATTCGTGAGCGATGTTAT |
| | TGAGTGACGTCATCCACGACTACAACGCGCAGGTC | CCACGACTATAACGCGCAAGTGGGTGCGCTGAACCTGC |
| | GGCGCGCTGAACCTGCCCGAGTGGCTGGCGCAGTA | CGGAATGGCTGGCGCAATACCGTGGCGAGCAGACCTTC |
| | TCGCGGCGAGCAGACGTTCTACAGTCTCTTCGATCT | TATAGCCTGTTTGATCTGTGGCTGGACCTGCTGCGTGCG |
| | GTGGCTGGACTTGCTGCGCGCCGGATTCGTGTGCG | GGTTTTGTTTGCGCGCCGAGCAGCGCGCGTCTGATGGA |
| | CGCCCAGCAGCGCGCGCCTTATGGAGCGCGTCTGC | ACGTGTTTGCTGGCTGGCGGATCTGCCGAGCCCGCGTG |
| | TGGTTAGCGGATCTGCCGTCGCCGCGCGCCCAGCT | CGCAGCTGCGTGATCAAATGCAGGAAGTTAACCCGGAC |
| | GCGCGATCAGATGCAAGAGGTCAACCCCGATTTCT | TTCTACACCGCGCTGAGCGAGAACGGTTTCCACCACTT |
| | ATACCGCACTCTCTGAGAACGGATTCCACCACTTC | TGTGGACACCGTGGTTCTGGGCAAGGAAATGCGTAGCA |
| | GTGGACACGGTGGTACTCGGCAAGGAGATGCGCTC | GCAAAAGCGAGCGTAGCTTTGTTCGTGATCTGACCACC |
| | GAGCAAAAGCGAGCGCTCGTTCGTGCGCGATCTGA | TGCGCGACCGATGCGGCGCAGGAATATGCGGAGCGTG |
| | CCACGTGTGCTACCGATGCAGCACAGGAATACGCG | AAGCGCGTACCATCTACCACGCGCTGTATGGTAGCGAT |
| | GAGCGCGAAGCGCGTACGATCTACCACGCCCTCTA | CGTACCGAGCAAGAACGTTACTGGCGTGAGCACTATGG |
| | CGGCAGCGACCGCACGGAACAGGAGCGCTACTGG | CGTTGACAAAACCCTGTTCCAGCCGACCACCCGTCGTA |
| | CGCGAGCACTATGGTGTTGATAAAACACTCTTTCA | ACTTCGCGGCGTACCCGGTGCCGGCGCTGCAACTGAGC |
| | GCCGACGACCCGCCGCAACTTTGCCGCATACCCGG | CCGGATGCGGCGCCGGGTGCGCTGCTGCAGCGTTATCG |
| | TGCCGGCTCTCCAGCTATCACCGGATGCAGCGCCC | TAGCCTGGTGCAAACCCAACTGAGCGCGCAGCAAGCG |
| | GGCGCACTGCTACAGCGGTACCGATCGCTGGTGCA | GAGCGTGTTGCGACCCAAGAAACCCAGCTGCTGGAGG |
| | GACGCAGCTGAGTGCACAGCAGGCAGAGCGTGTTG | ATATGCTGGGTATCGACAACAACGCGAACGCGCTGAGC |
| | CCACGCAGGAGACGCAGCTCTTGGAGGACATGCTC | AACGTGTTCAACGAGTTTCTGCGTGAAGTTCGTACCGA |
| | GGTATCGATAACAACGCCAACGCGCTCTCGAACGT | GACCGGTCGTGCGGCGATTGCGGACGATATGCAGCAAT |
| | ATTCAACGAGTTTCTCCGCGAGGTGCGTACCGAGA | TCAGCCGTGCGTGGGATGGTCGTCGTAGCGAACTGGAG |
| | CAGGCCGTGCTGCGATCGCTGACGATATGCAGCAG | GAACGTCTGCGTTGGCTGGGCGAACGTGCGGCGCAACT |
| | TTCAGTCGCGCGTGGGACGGACGACGCTCGGAGTT | GCCGGCGCAGCCGCGTCTGGCGAACAGCTGGGCGGACT |
| | GGAAGAGCGCCTGCGCTGGCTCGGCGAGCGTGCGG | ACCGTACCAGCGTTGCGGGCAAGCTGCAAAGCTGGGTT |
| | CGCAGCTGCCGGCGCAGCCGCGGCTGGCGAATAGC | AGCAATGTTGCGCGTCAGGAACACGTGATCCGTCCGCG |
| | TGGGCGGACTACCGCACCAGCGTGGCCGGCAAACT | TCTGGAACAGCAACGTAGCGAGCTGGACGATCTGGCGG |
| | CCAGAGCTGGGTGTCGAACGTGGCACGGCAAGAG | AACGTCTGCGTGCGCTGAGCGATGAGGAAACCGGTCTG |
| | CACGTCATCCGTCCGCGACTGGAGCAGCAACGCAG | CCGGCGACCGTTGAGCAAGCGCAAGCGGCGCTGGATG |
| | TGAGCTCGACGACCTGGCCGAGCGGCTACGCGCGC | CGGCGCTGGCGGCGGAACAGAGCGACGAGAGCACCCT |
| | TCAGCGATGAGGAGACCGGGCTGCCGGCTACCGTT | GATGGTGTATCGTGATGCGCTGGCGGATGTTCGTGCGG |
| | GAGCAGGCACAGGCAGCGCTCGACGCCGCGCTGG | CGCTGAACGAGGGTCAACACACCCTGCAGATGCACGA |
| | CGGCAGAGCAATCGGATGAGTCGACGCTGATGGTC | ACACGGCATTGAGCACGTGGACACCGATAGCAGCTGG |
| | TACCGCGATGCGCTCGCTGACGTGCGTGCGGCACT | GCGAGCGATACCTGGCCGACCCTGCACCAACCGGTGCC |
| | CAATGAAGGTCAGCATACGCTGCAAATGCACGAGC | GCAAGTTCCGCAGTTTCCGGGTGTGACCAAGGCGTACG |
| | ACGGCATCGAACACGTGGACACTGACAGCAGCTGG | CGTATACCAAATACGTTCACGCGCTGGAACTGCTGCGT |
| | GCATCGGACACGTGGCCGACGCTCCACCAGCCGGT | AGCGGTGCGGCGGTGCTGGAGCGTGCTGCGGCGGACG |
| | ACCGCAGGTGCCCCAGTTCCCGGGCGTGACGAAGG | CGAGCGAGCGTGAAGCGGTTCAGCTGAGCCGTGAGGA |
| | CGTACGCGTACACGAAGTACGTGCACGCGCTCGAG | AATGCTGCGTCGTCTGACCAACGTGGCGCAGCAATATG |
| | CTGTTGCGCAGCGGTGCTGCCGTACTTGAGCGGGC | CGCGTTGCAACAGCCAACGTTTCCGTGATCTGATCGGT |
| | CGCCGCCGATGCCAGTGAGCGGGAGGCCGTTCAGC | GGCGTGTTTCAGCGTCACGAAGTTCTGCTGAACGACGT |
| | TCTCGCGCGAGGAGATGCTGCGCCGCCTGACGAAC | GGTTGAGCGTGGTGCGGTTTACTATCAAAGCCCGCGTG |
| | GTGGCGCAGCAGTACGCACGCTGCAACAGCCAGCG | CGCGTAACAAGAAACCGCTGGTTGAGCTGAGCCACACC |
| | GTTCCGTGACCTGATCGGTGGCGTATTCCAACGGC | GATGAGCAGCTGCACGCGGTGATCACCGACCTGGTTTG |
| | ACGAGGTGCTACTCAACGATGTTGTTGAACGGGGA | GAAATGCGCGCCGTACTGGGAACGTATGTGGGGTCAAA |
| | GCGGTGTACTACCAGTCGCCGCGCGCCCGCAACAA | TCGAGGAAGTGGTTGATGCGATTGACTTCGAGCGTGTT |
| | GAAGCCGCTGGTTGAACTGAGTCACACCGACGAGC | CGTCTGGGCATGCTGTGCGCGCTGTATCCGGATACCAC |
| | AGTTGCACGCGGTGATCACCGATCTCGTCTGGAAG | CGCGGATATTAGCGACGTGAGCGAAACCCTGTTTACCC |
| | TGTGCGCCGTACTGGGAACGCATGTGGGGGCAGAT | GTGCGGGTGGCTACCAGCGTGCGTATGGTACCGAGCTG |
| | CGAGGAGGTCGTCGATGCGATTGACTTTGAGCGCG | ACCGGCACCACCCTGAGCAACTGCATCCAACGTGTTAT |
| | TCCGGCTCGGCATGCTCTGTGCGCTGTATCCGGAC | TCTGGCGGAAATGAAGGGCGCGGCGCAGCGTATGAGC |
| | ACCACTGCCGATATTAGTGATGTGTCAGAGACGCT | CGTGAGTGGTTCGTGGTTCGTTACACCGTGCAAATCGTT |
| | GTTCACCCGAGCTGGCGGGTACCAGCGCGCCTACG | AAGGCGGACGAGCTGTACCCGCTGATTTATCAACCGGG |
| | GCACTGAGTTGACCGGCACCACGCTCTCGAATTGT | TAGCACCGGTGGCCGTGGTACCTGGCACATCACCGATC |
| | ATACAGCGGGTCATTCTAGCGGAGATGAAAGGCGC | GTCAAAACGTTCGTCGTAGCGCGGCGGACACCCCGCCG |
| | GGCGCAGCGGATGAGCCGTGAGTGGTTTGTGGTGC | GTGTACCGTAAGGTTGGTAAAAACCTGCCGCACGATAC |
| | GCTACACGGTGCAGATCGTCAAAGCGGACGAGCTG | CGCGCTGGCGGGTTTTGATGGTGCGGAAGTGACCGACA |
| | TATCCGCTGATCTATCAACCCGGCTCTACGGGCGG | CCCAGCGTCTGCTGAGCATTCGTAGCAGCCGTTATCAA |
| | CCGCGGCACATGGCACATCACCGATCGACAGAACG | CTGCAGTTTCTGCAAGATCAACTGCATGCGGGTAGCGA |
| | TGCGTCGAAGTGCAGCAGACACGCCGCCGGTGTAC | GCACATGCGTCGTCGTTTCAGCTGGAGCATCGCGGAAT |
| | CGGAAAGTCGGGAAGAACCTCCCGCACGACACCG | ACAGCTTTATTTGCGAGGATACCTATACCGCGGCGTGG |
| | CGCTTGCCGGTTTCGACGGCGCAGAAGTAACTGAT | GACACCGAACGTGGTACCGTTAGCCTGGAGCGTCAACC |
| | ACGCAGCGTCTCCTCTCGATTCGCAGCTCGCGCTAT | GAGCGCGCGTCGTCTGTTCGTTAGCATCCCGTTTCAACT |
| | CAGCTACAGTTCTTGCAAGACCAGCTTCACGCCGG | GCGTCGTCTGGAAGCGGCGGATGGCCGTAGCAGCTACC |
| | CAGTGAACACATGCGGCGACGTTTCAGCTGGAGCA | AGCCGAAGAGCGGTCTGCCGTACAGCTATCTGCTGGGC |
| | TCGCCGAGTACTCATTCATTTGTGAGGATACGTATA | CTGGACGTGGGTGAATACGGCATTGCGTATTGCCTGCT |
| | CGGCCGCGTGGGATACAGAGCGCGGCACCGTTTCG | GGAGCCGGAAACCGGCGAGTGGCGTACCAGCGGCTTCT |
| | CTCGAGCGGCAGCCGAGCGCTCGTCGTCTGTTCGT | TTGCGGACGATGCGATCCGTAAAATTCGTCAGTACGTG |
| | TTCCATTCCGTTCCAGCTGCGGCGGCTAGAAGCCG | AGCCGTCAAAAAGAGGCGCAGGTTCGTAGCACCTTTAG |
| | CTGATGGTCGATCGTCCTATCAGCCAAAGAGCGGC | CGCGCCGAGCAGCGAACTGGCGCGTATCCGTGAGAAC |
| | TTGCCGTACAGCTACCTGTTGGGGCTCGACGTGGG | GCGATTACCGCGCTGCGTAACCGTGTGCACGATCTGAC |
| | TGAGTACGGTATCGCGTACTGCCTGCTAGAGCCGG | CGTTCGTTACGACGCGCGTCCGGTTTATGAATTCAACAT |
| | AGACCGGCGAGTGGCGGACGAGCGGTTTCTTTGCA | CAGCAACTTTGAGAGCGGTAGCAACCGTGTGGCGAAG |
| | GACGATGCGATACGCAAGATCCGCCAGTACGTTTC | ATTTACCGTAGCGTGAAAACCGCGGATGTTCACGCGGA |
| | CAGGCAGAAAGAGGCACAGGTACGCAGCACTTTC | CAACGATGCGGACCAGGCGGAACGTGACCTGGTTTGGG |
| | AGTGCGCCGTCGTCAGAACTTGCACGTATCCGCGA | GTAGCGCGAGCAAACTGACCGGCAGCGAGATCGGTGC |
| | GAACGCGATCACCGCGCTACGCAATCGCGTGCACG | GTACGGCACCAGCTATGTGTGCAGCAAGTGCCACGCGA |
| | ATCTGACCGTACGCTACGATGCGCGGCCGGTGTAC | GCCCGTACACCGCGATTCAACCGATGCAGCAAAGCGCG |
| | GAATTCAATATCTCTAACTTTGAGAGTGGTTCTAAT | TATGAGTGGGAATGGGTGGGTCAGCAACAGCGTATCGT |
| | CGCGTTGCCAAGATCTATCGGTCCGTCAAAACCGC | TCGTATTTATACCCCGGAAAACGGTGCGGCGCTGGGTC |
| | TGATGTGCACGCTGACAACGATGCGGATCAAGCGG | ACATCGATATTCGTCAGTATAAACCGAGCGATACCCTG |
| | AGCGCGACCTCGTGTGGGGTAGTGCCAGCAAGCTG | CCGAGCGTTGACGCGCTGCGTTTCCTGAAAGCGTACGC |
| | ACCGGCAGCGAGATCGGGGCGTACGGTACCAGTTA | GCGTCCGCCGCTGGAGGCGCTGGTGCAACGTAGCGGTT |
| | CGTATGCAGCAAGTGTCACGCCTCGCCGTATACGG | TTACCGATCAGGACACCATCGATCGTCTGCACGCGTAC |
| | CTATTCAACCAATGCAGCAATCCGCATACGAGTGG | GTGCAGGAACGTGGCGACAGCGCGGTTTATACCTGCCC |
| | GAGTGGGTTGGTCAGCAGCAGCGGATCGTGCGCAT | GTTCTGCGAGCACACCGCGGATTGCGATGTGCAAGCGG |
| | TTACACACCTGAAAACGGTGCTGCGCTTGGGCACA | CGCTGATTGTGGCGGTTAAGTACGCGATTAAACAGCAC |
| | TCGATATTAGACAGTACAAGCCAAGTGATACGTTG | GGTAGCCCGAGCGGCGAGAAAGGCGAAGTGACCCTGG |
| | CCGTCGGTGGATGCACTCCGCTTTTTGAAGGCGTA | AAGACGTTAGCGCGTATCTGCGTGGCCACGAGGTGCAG |
| | CGCGCGGCCGCCGCTCGAGGCGCTCGTACAGCGTT | CCGGTTAGCTTTGCGTAG (SEQ ID NO: 32) |
| | CGGGCTTTACGGATCAGGACACGATAGACCGGCTC | |
| | CACGCGTACGTACAAGAGCGTGGTGACAGTGCGGT | |
| | GTACACCTGCCCGTTCTGTGAGCACACAGCAGATT | |
| | GCGATGTGCAGGCAGCGCTCATCGTTGCTGTGAAG | |
| | TATGCGATCAAGCAGCACGGATCGCCGAGTGGCGA | |
| | GAAGGGTGAAGTGACGCTGGAAGACGTTAGCGCA | |
| | TACCTCCGTGGTCACGAGGTGCAGCCCGTCTCATTC | |
| | GCATAATAG (SEQ ID NO: 31) | |
| Type V | ATGAGGAGACAATTAGAAGATTTTGCCAATCYTTA | ATGCGCCGTCAACTGGAGGACTTTGCGAACCTGTATGA |
| Cas_7 | TGAAATTTCCAAAACCTTGCGTTTTGAATTGAGGCC | GATTAGCAAGACCCTGCGCTTTGAACTGCGTCCGATTG |
| | TATTGGAAAAACGCGTAAAATGCTTGAGGAAAATA | GTAAAACCCGTAAGATGCTGGAGGAAAACAAAGTGTTT |
| | AAGTATTTGAAAAAGATGAGGCAGTAGCTCAAAAT | GAGAAGGACGAAGCGGTTGCGCAGAACTACCAAGAGG |
| | TACCAAGAAGCAAAAAAATGGCTGGATAAATTGC | CGAAGAAATGGCTGGATAAACTGCACCGTGACTTCATT |
| | ATAGAGATTTTATTAGCCGCTCTCTTGAGGATTTAA | AGCCGTAGCCTGGAGGATCTGAAGATCAACAGCGAACT |
| | AAATAAATTCCGAACTTCTGGAAGAACACAAACAG | GCTGGAGGAACACAAACAAGCGTACTTTGACTATAAGA |
| | GCTTATTTTGACTACAAAAAAGAAAAAAATTCTTC | AAGAAAAGAACAGCAGCAACCGTAACAACTTCGAGGA |
| | CAACAGAAATAATTTTGAAGAAAAATCCAAAAAG | AAAGAGCAAGAAACTGCGTAAAGAGATCCTGCTGAAC |
| | CTGAGAAAAGAAATTTTATTGAATTTTTGCCAAAA | TTTTGCCAGAAAGGCGAGGAACTGCGTGATAACTACCT |
| | AGGAGAAGAATTGAGAGATAATTACTTGAGAGAA | GCGTGAGATCAAAGACGAAAAGATTAAGAAACGTGTT |
| | ATAAAAGATGAAAAAATCAAAAAGAGAGTTCGAA | CGTAAGCTGCGTAACCTGGATATTCTGTTCAAGGTTGA |
| | AGCTGAGAAACTTGGATATTCTTTTTAAAGTGGAA | GGTGTTCGACTTTCTGAAACAGCGTTATCCGGAGGCGG |
| | GTTTTTGATTTTTTAAAACAAAGATACCCGGAAGCT | TGGTTGATGAGAAGAGCATCTTCGATGCGTTCAACCGT |
| | GTTGTTGACGAGAAAAGTATTTTCGATGCCTTTAAT | TTCAGCACCTACTTTACCGGCTTCCACGAAACCCGTAA |
| | AGATTTAGTACTTATTTTACAGGTTTCCACGAGACA | AAACTTTTATAAGGATGATGGCACCGCGACCGCGATCC |
| | AGAAAAAATTTCTATAAAGACGACGGTACTGCCAC | CGACCCGTATTGTGAACGAGAACCTGCCGAAGTTCCTG |
| | CGCTATTCCTACCAGAATTGTAAATGAAAACCTAC | GATAACCTGGAAGTGTACAACCGTTACTATAAAGAAGG |
| | CCAAGTTTCTTGATAATTTGGAAGTTTACAATAGAT | TATTGGCGACCTGTTTACCGGCGAGGAAAAGAACATCT |
| | ATTACAAAGAAGGCATTGGAGATTTGTTTACAGGA | TCAACCTGGAGTTCTTTAACGATTGCTTTAGCCAGCGTG |
| | GAAGAAAAAAATATTTTCAACTTGGAATTTTTTAAT | AAATTGACAGCTATAACCGTATCATTAGCGAGATCAAC |
| | GATTGTTTTTCTCAAAGAGAGATTGATTCTTACAAC | CTGAAAATTAACCAGAAGCGTCAAACCGCTGAGAATA |
| | AGAATTATTTCCGAAATAAATTTAAAAATTAACCA | AGAAAAACTTCCCGTTTCTGAAAACCCTGTTCAAGCAG |
| | AAAACGCCAAACAGCGGAAAATAAGAAAAATTTT | ATCCTGGGTGAGGAAGAGAAGCAAGAAACCGAAAGCC |
| | CCCTTTCTTAAAACGCTTTTCAAGCAAATTTTGGGA | TGGATTACATCGAGATTACCCGTGACGAAGATGTGTTT |
| | GAAGAAGAGAAACAGGAAACCGAGTCTCTTGATT | CCGGCGCTGAAGAGCTTCGTTGAAGAGAACGAACGTCA |
| | ATATAGAGATAACCCGGGATGAAGACGTGTTTCCG | GACCCCGCGTGCGAACAAGCTGTTTAACCGTCTGATTC |
| | GCTTTGAAGAGCTTTGTAGAAGAAAACGAGAGGCA | AGGATCAAAAAGAGCAAAAGGGTGGCTTCGACATCAG |
| | AACTCCTAGGGCCAATAAGCTTTTCAACAGGTTAA | CAACGTGTTTGTTGCGGGTCGTTTCATCAACCAGATTAG |
| | TTCAAGATCAAAAAGAGCAAAAAGGCGGTTTTGAT | CAACAAATACTTTGCGGACTGGAACACCATCCGTAGCA |
| | ATTTCCAATGTTTTTGTAGCTGGTAGATTTATTAAT | TCTTCATTGAGAAGGGCAAGAAAAAGCTGCCGGAATTT |
| | CAGATTTCCAATAAATACTTTGCAGACTGGAACAC | GTGAGCCTGCAGGAGCTGAAAGAAAAGCTGCAAAGCA |
| | CATTAGAAGTATTTTTATTGAAAAGGGAAAAAAGA | TCGAGATTGAGAAGAGCGAGCTGTTCCGTGAAAAGTAC |
| | AATTACCGGAGTTTGTTTCTCTGCAAGAGCTCAAA | AAGGATATTTACAAGAACCGTGGCGACAACTTTATCAT |
| | GAAAAACTCCAAAGCATAGAGATAGAAAAAAGCG | CTTCCTGGAAATCTGGCAAAAGGAGTTCGAAGAGAGCC |
| | AATTATTTAGAGAGAAGTATAAAGATATATATAAA | TGAAACGTTACCGTGAAAGCCTGGAAGAAACCAAACA |
| | AACCGAGGGGATAATTTTATTATCTTTCTTGAGATA | GATGCTGGAGCAGCAAGAAGGTTACCAGAGCAAGGAG |
| | TGGCAAAAAGAATTTGAAGAGAGCCTAAAAAGAT | AGCAGCGAACAGAAGAACAGCATCCGTCGTTATTGCGA |
| | ACAGAGAAAGCTTGGAAGAAACCAAGCAAATGCT | GAACGCGCTGAGCATCTACCAAATGATTAAGTATTTCA |
| | TGAGCAGCAAGAAGGCTATCAAAGCAAGGAAAGT | GCCTGGAGAAAGGCAAGGAACGTGTTTGGAACCCGGA |
| | TCCGAACAGAAAAACTCAATTCGCCGTTATTGTGA | TAAACTGGAAGAGGACCCGGGCTTTTACGAACTGTTCA |
| | AAATGCGCTCTCTATTTATCAAATGATAAAGTATTT | AGGATTACTATCAGGACGCGCACACCTGGCAATACTAT |
| | TTCCCTGGAAAAAGGCAAGGAAAGGGTTTGGAATC | AACGAGTTTCGTAACTACCTGACCAAAAAGCCGTATAG |
| | CGGACAAACTGGAAGAAGACCCCGGATTTTACGAG | CCAGGATAAAGTGAAGCTGAACTTTGGTAGCGGCACCC |
| | CTTTTCAAGGACTATTACCAAGATGCTCATACTTGG | TGCTGCAGGGTTGGCCGGACAGCCCGGAGGGTAACACC |
| | CAATACTATAACGAATTTCGAAACTATTTAACCAA | CAATACAAAGGCTTCATCTTCAAAAAGAACAAGAAGTA |
| | AAAGCCTTATAGTCAAGATAAGGTTAAATTGAATT | CTTTCTGGGCATCACCAACTATCCGAAAATGTTCAACG |
| | TTGGAAGCGGAACCTTATTGCAAGGGTGGCCAGAT | AGAAGCGTCACCCGGAAGCGTACGACAACGATATTGA |
| | AGTCCGGAAGGCAATACCCAGTATAAAGGTTTTAT | CCCGTACTACAAGATGATCTACAAGCAGCTGGATAGCA |
| | TTTTAAAAAAAATAAAAAATATTTTTTAGGCATAA | AAACCATCTTTGGTAGCCTGTACCTGGGTAAATTCGGC |
| | CAAATTATCCTAAGATGTTTAATGAAAAGCGTCAC | AACAAATATAAAGAGGACAAAAAGCGTATGGTGGACT |
| | CCTGAAGCTTATGATAATGATATTGATCCTTATTAT | TCAAGCTGCAAAACCGTATCCGTGCGATTCTGAAAGAG |
| | AAGATGATTTACAAACAATTAGACAGCAAAACCAT | AAGGTTGAGTTCTTCCCGCGTCTGCAGACCATCATTGA |
| | ATTCGGTTCTTTGTATTTAGGAAAATTTGGAAATAA | CAAAATTGAAAACCACAAGTACAGCAACACCAAAGAC |
| | GTACAAAGAAGATAAAAAAAGAATGGTTGACTTTA | ATCGCGGTGGACATCAGCAAGATCAAGCTGTACAACAT |
| | AGCTACAAAACAGGATAAGAGCTATATTAAAAGA | CTTCTTTATCGAAACCAACAGCCTGTACGTTGAGCAGG |
| | GAAGGTCGAGTTTTTCCCTCGATTGCAAACCATTAT | GTAAGTACGAAATCGATAACAACACCAAGAACCTGTAC |
| | AGATAAAATTGAAAATCATAAATATTCGAATACAA | CTGTTTGAAATCTATAACAAAGACTTCGCGAAAAAGGC |
| | AGGATATTGCTGTGGATATTTCTAAGATAAAGTTAT | GGAGGGCAAAAAGAACCTGCACACCTACTATTGGGAA |
| | ACAACATTTTTTTTATAGAAACAAACTCTTTGTATG | GAAATCTTCAGCCAGCGTAACCAAGACAACCCGATCAT |
| | TTGAACAAGGTAAGTATGAGATAGACAATAATACA | TAAACTGAACGGTCAGGCGGAAGTGTTCTTTCGTCGTG |
| | AAAAATTTGTATCTCTTTGAAATTTACAACAAAGAT | CGAGCCTGGACCCGGAAGTGGACGAAGAGCGTAAGGC |
| | TTTGCAAAGAAGGCAGAAGGAAAAAAGAATCTGC | GCCGCGTGAGGTGGTTAACAAGGAGCGTTACACCGAA |
| | ACACCTATTACTGGGAGGAGATTTTTTCCCAAAGA | GATAAAATGTTCTTTCACTGCCCGCTGACCCTGAACTTT |
| | AATCAAGATAATCCGATCATCAAATTAAACGGCCA | GCGAAGGGTCGTGCGGACGGCTTCAGCATTAAAGCGCG |
| | AGCCGAGGTATTTTTCAGAAGAGCCTCTTTGGATC | TGAATATCTGCTGGAGAACCCGGAAGTGAACATCATTG |
| | CGGAAGTTGACGAAGAAAGAAAAGCGCCTCGGGA | GTATCGACCGTGGCGAGAAACACCTGGCGTACTATAGC |
| | AGTTGTAAATAAAGAAAGATACACTGAAGACAAA | GTTGCGGATCAAGAGGGCAACATCCTGGAAATTGACAG |
| | ATGTTTTTTCATTGTCCCTTGACGCTTAATTTTGCCA | CCTGAACAAGATCAACGAGGTTGATTACCACAAAAAGC |
| | AAGGTCGAGCGGATGGGTTTAGTATAAAGGCGAGG | TGGACAAACTGGAGAAGGCGCGTGATGAAGCGCGTAA |
| | GAGTATTTGCTCGAAAATCCGGAGGTGAACATTAT | AACCTGGCAGGACATCGCGAAGATCAAGGAAATGAAG |
| | CGGCATCGATCGGGGGGAAAAGCATTTAGCCTATT | CAGGGTTACATCAGCCAAGTGGTGAAGAAAATCTGCGA |
| | ATTCCGTAGCGGACCAAGAAGGGAATATTTTGGAA | TCTGATGATTAAACACAACGCGATCGTGGTTTTCGAGG |
| | ATAGATTCCCTTAATAAAATCAATGAAGTTGACTA | ACCTGAACCTGGGTTTTAAGTGCGGCCGTTTCGCGATC |
| | TCATAAAAAGCTTGATAAGTTGGAAAAAGCAAGGG | GAGAAACAGGTGTACCAAAACCTGGAACTGGCGCTGG |
| | ATGAGGCTCGCAAAACTTGGCAGGATATAGCCAAG | CGAAAAAGCTGAACTATCTGGTTTTTAAAGAGCGTGAA |
| | ATCAAAGAAATGAAACAAGGATATATTTCCCAGGT | GCGGAAGAGCTGGGCAGCTTTCGTCATGCGTTCCAGCT |
| | TGTAAAGAAAATTTGCGACTTAATGATAAAACACA | GACCCCGCAAATTAGCAACTTCAAGGACATCAAGAAGC |
| | ATGCTATAGTGGTTTTTGAAGATCTCAACCTCGGCT | AGTGCGGTTTCATGTTTTACATTCCGGCGCGTTATACCA |
| | TTAAGTGCGGAAGATTTGCCATAGAGAAGCAGGTT | GCGCGATCTGCCCGAACTGCGGCTTTCGTAAGAACATT |
| | TATCAAAACTTGGAGCTGGCTTTGGCCAAAAAATT | AGCACCCCGGTGGACAAAAAGGCGAAAAACAAGGAGT |
| | GAATTATTTGGTTTTCAAAGAGAGGGAAGCGGAGG | ACCTGGAAAAATTCCAGATCAGCTATGAACAAGATCGT |
| | AGCTTGGCAGTTICAGGCATGCGTTTCAATTAACTC | TTCAAGTTTGCGTACAAAAAGCGTGACGTTCTGGAGCG |
| | CTCAAATATCTAATTTCAAAGATATTAAAAAACAA | TGGTCGTGGCAACCCGGGTCAGAACAGCCGTCGTCTGT |
| | TGCGGTTTTATGTTTTATATTCCTGCCAGATACACC | TTGAAGAGAAAGCGAGCAAGGACGATTTCATCTTCTAC |
| | TCCGCTATTTGCCCTAACTGCGGTTTCCGCAAAAAT | AGCGATGTGAGCCGTCTGCAGTTCCAACGTAACAAGGA |
| | ATTTCCACTCCCGTTGACAAAAAAGCTAAAAACAA | CAACCGTGGTGGTGAAACCAAATGGCGTGAACCGAAC |
| | AGAATATCTTGAAAAGTTTCAAATTTCTTACGAGC | GAAGAGCTGAAACGTATCTTCAAGGAGAACGGTATCG |
| | AAGATAGATTTAAATTTGCTTACAAGAAAAGAGAT | ATATTAACAAGGACATCAACAAGCAGATCAAAGAGGG |
| | GTCCTTGAGAGAGGGAGGGGAAACCCCGGTCAAA | TGATTTTGAAAACGATGCGTTCTACAAGCGTATCATTC |
| | ATAGCCGGCGCCTTTTTGAGGAAAAAGCTTCAAAA | ACACCATCCGTCTGATTCTGCAGCTGCGTAACGCGATC |
| | GATGATTTTATTTTCTACTCCGATGTTTCCAGATTA | ACCAAAAAGGATGAGCAAGGCAACGAAATTGAAGAGG |
| | CAGTTTCAAAGAAATAAAGACAATCGGGGAGGCG | AAAGCCGTGACTTTATCCAATGCCCGAGCTGCCACTTC |
| | AAACAAAGTGGCGCGAGCCGAACGAAGAGCTGAA | CACAGCGAAAACAACCTGCTGGCGCTGAGCGAGAAAT |
| | GAGAATTTTCAAAGAAAACGGGATTGACATCAATA | ACAAGGGTGATGAACCGTTCCAGTTTAACGGTGACGCG |
| | AAGACATTAACAAGCAAATCAAAGAAGGAGATTTT | AACGGCGCGTATAACATCGCGCGTAAAGGTAGCCTGAT |
| | GAAAATGACGCTTTCTACAAGAGAATTATTCACAC | CCTGAGCAAGATTAGCAACTTCAACAAAACCGAGGGC |
| | CATTCGTTTAATATTGCAATTGAGAAACGCCATAA | GACCTGAGCAAGATGGATAATCAAGACCTGACCATCAC |
| | CAAAAAAAGACGAGCAAGGAAATGAAATTGAAGA | CCAAGAAGAGTGGGACAAGTTCGCGCAGAATAAATAG |
| | AGAAAGCCGGGATTTTATTCAGTGCCCCTCTTGTCA | (SEQ ID NO: 34) |
| | TTTTCATTCAGAAAACAATCTTTTGGCCTTAAGCGA | |
| | GAAATACAAAGGGGATGAACCGTTTCAATTCAACG | |
| | GCGATGCCAATGGAGCATATAACATAGCTCGCAAG | |
| | GGAAGTCTTATTTTAAGCAAGATTTCAAATTTTAAC | |
| | AAAACAGAGGGTGATTTAAGCAAAATGGATAACC | |
| | AAGATTTGACCATTACCCAAGAAGAATGGGATAAA | |
| | TTTGCGCAAAATAAATAG (SEQ ID NO: 33) | |
| Type V | ATGTCTGTTCGCGCAATCCGTGCCCGCTCGCCTGC | ATGAGCGTTCGTGCGATCCGTGCGCGTATTGCGTGCGA |
| Cas_8 | GATCGGACTGTACTCGATCACCTCTGGCGCACCCA | TCGTACCGTGCTGGACCACCTGTGGCGTACCCACTGCG |
| | TTGTGTCTTTCACGAGCGGCTGCCGATTGTGCTGGG | TTTTCCACGAACGTCTGCCGATTGTGCTGGGCTGGCTGT |
| | CTGGCTTTTCCGCATGCGACGAGGCGAATGCGGCG | TTCGTATGCGTCGTGGCGAGTGCGGTGAAACCGATGCG |
| | AGACTGATGCCGAGCGACTCCTTTACCAGCGCGTC | GAGCGTCTGCTGTACCAGCGTGTTGGCAAATTCATCAC |
| | GGCAAGTTCATTACTGGCTATTCCGCCCAGAACGC | CGGTTACAGCGCGCAAAACGCGGACTATCTGATGAACG |
| | TGACTACCTAATGAACGCGGTCAGCCTGAAAGGCT | CGGTGAGCCTGAAGGGTTGGAAACCGGCGACCGCGAA |
| | GGAAGCCGGCCACCGCCAAGAAATACAAGATTAA | GAAATATAAGATTAAAACCGACGATGACAACGGCCAG |
| | GACCGACGACGACAACGGTCAGTCGGTCCAGATCA | AGCGTTCAAATCAGCGGTGAAAGCTGGGCGGATGAGG |
| | GCGGCGAGTCGTGGGCCGATGAGGCTGCTGCCCTT | CTGCGGCGCTGAGCGCGCAGGGTAAACTGCTGTTTGAC |
| | TCGGCCCAAGGAAAGCTACTCTTCGACAAGAACGT | AAGAACGTGGTTAGCGGTGGCCTGCCGGGTTGCATGCG |
| | GGTTTCGGGTGGCCTGCCCGGATGTATGAGACAGA | TCAAATGCTGAACCGTGAAAGCGTGGCGATCATTAGCG |
| | TGCTCAATCGAGAATCCGTCGCCATTATCAGCGGC | GCCACGATGAGCTGCTGAGCAAGTGGAACACCGACCA |
| | CACGACGAACTGCTGTCCAAGTGGAACACAGACCA | CACCAAATGGCTGGGTGAAAAGGCGCAGTGGGAAGCG |
| | CACCAAGTGGCTCGGCGAGAAAGCCCAATGGGAA | GTTCCGGAGCACACCCTGTACCTGGCGCTGCGTAAGAA |
| | GCCGTTCCTGAACACACGCTCTACCTCGCGCTTCGC | ATTCGAGAGCTTTGAACAAGCGGTGGGTGGCAAGGCG |
| | AAAAAGTTCGAGTCCTTTGAACAAGCCGTTGGCGG | ACCAAACGTCGTGGTCGTTGGCACCGTTATCTGGATTG |
| | TAAGGCGACCAAGAGGCGAGGGCGTTGGCACCGC | GCTGCGTGCGAACCCGGACCTGGCGGCGTGGCGTGGTG |
| | TATCTCGACTGGTTGCGCGCCAATCCTGATTTGGCC | GCCCGGCGATTGTGGATGAGCTGAGCCCGGCGGCGCAG |
| | GCTTGGCGCGGCGGGCCCGCGATTGTCGACGAACT | GAGCGTATCCGTAAGGCGAAACCGTGGAAGAAACGTA |
| | GTCACCCGCTGCGCAAGAACGTATCCGCAAGGCCA | GCGCGGAAGCGGAGGAATTCTGGAAAATTAACCCGGA |
| | AACCATGGAAGAAACGGTCCGCCGAGGCGGAAGA | GCTGGCGAGCCTGGATAAGCTGCACGGCTACTATGAGC |
| | GTTCTGGAAGATCAATCCCGAGCTTGCCTCGCTCG | GTGAATTTGTTCGTCGTCGTAAGAACAAACGTAACCCG |
| | ACAAGCTCCACGGTTACTATGAGCGCGAGTTCGTT | GATGGTTTCGACCACCGTCCGACCTTTACCATGCCGGA |
| | CGCCGGCGCAAGAACAAACGCAACCCCGATGGTTT | CCGTATCCGTCACCCGCGTTGGTTCGTGTTTAACGCGCC |
| | TGATCACCGGCCAACGTTCACCATGCCCGACCGGA | GCAGACCAACCCGAGCGGTTACCGTCACCTGCGTCTGC |
| | TTCGGCACCCGCGCTGGTTTGTTTTCAACGCACCGC | CGCAAGGCGCGAAAGAGATCGGTGCGGTTCAGCTGCA |
| | AGACGAATCCATCCGGATATCGCCATCTGCGCTTG | ACTGATTACCGGTGGCCGTGAGGGCGAAGGTGTGTACC |
| | CCTCAAGGCGCCAAAGAAATCGGCGCCGTGCAGCT | CGACCCAGTGGGTGGATGTTACCTATCGTGCGGACCCG |
| | CCAGCTAATCACCGGCGGGCGCGAAGGCGAGGGC | CGTCTGGCGCTGTTCCGTCGTAGCCAGGTGAGCACCAC |
| | GTGTACCCAACGCAATGGGTCGACGTGACGTATCG | CGTTAACCGTGGCAAGGCGAAAGGTCAAACCAAGATT |
| | CGCCGACCCGCGCTTGGCGCTGTTCCGCCGGTCGC | AAAGAGGGTTACGAATTCTTTGATCGTCACCTGAGCCA |
| | AAGTGTCGACCACAGTCAATCGGGGGAAAGCGAA | ATGGCGTAGCGCGGAAATCAGCGGCGTTAAACTGATCT |
| | AGGACAGACAAAGATCAAGGAAGGCTACGAGTTC | TCCGTGACATTCGTCTGAACGATGACGGTAGCCTGAAG |
| | TTTGACCGGCATCTGAGCCAATGGCGGTCCGCGGA | AGCGCGATCCCGTATCTGGTGTTTGCGTGCAGCATTGA |
| | GATCAGCGGCGTCAAACTGATCTTCCGCGACATCC | TGACCTGCCGCTGACCGAGCGTGCGAAGAAAATTGAGT |
| | GGCTTAATGACGACGGCTCACTGAAGTCGGCTATT | GGAGCGAAACCGGCGAAACCACCAAAACCGGTAAGAA |
| | CCCTACCTGGTGTTCGCGTGCAGCATTGATGATCTT | ACGTAAAAGCCGTACCCTGCCGGATGGCCTGATTGCGT |
| | CCACTTACTGAGCGGGCCAAGAAGATCGAATGGTC | GCGCGGTGGACCTGGGCCTGCGTAACGTTGGTTTCGCG |
| | TGAGACGGGCGAGACGACAAAGACCGGGAAGAAA | ACCCTGTGCGTGTTTGAACACGGCAAGAGCCGTGTGCT |
| | CGAAAATCCCGCACGCTGCCCGACGGGCTCATCGC | GCGTAGCCGTAACATTTGGCTGGATGATGAGGGTGGCG |
| | GTGTGCCGTGGATCTGGGGTTACGCAACGTCGGCT | GTCCGGATCTGGGTCACATCGGTCAGCACAAACGTCAA |
| | TTGCTACACTCTGTGTCTTTGAACACGGAAAGTCAC | ATTAAGCGTCTGCGTCGTAAGCGTGGCAAACCGGTTAA |
| | GCGTCCTGCGGTCGCGCAATATCTGGCTGGATGAT | GGGTGAACTGAGCCACGTGGAGCTGCAGGATCACATCA |
| | GAGGGTGGTGGCCCCGACCTGGGACACATCGGCCA | CCCACATGGGCGAGGACCGTTTCAAGAAAGCGGCGCGT |
| | GCACAAACGACAGATCAAGCGACTGCGCCGCAAG | GGTATCATTAACTTTGCGTGGAACGTGGATGGTGCGGT |
| | CGCGGCAAGCCGGTCAAGGGCGAACTCTCACACGT | TGATGAAGCGACCGGCGAGCCGTTCCCGCGTGCGGATG |
| | GGAGTTGCAGGACCACATTACACACATGGGAGAA | CGATCGTTCTGGAAAAACTGGAGGGCTTTATTCCGGAC |
| | GACCGTTTCAAGAAGGCAGCGCGCGGCATCATCAA | GCGGAGAAGGAACGTGGTATCAACCGTAGCCTGGCGG |
| | CTTCGCTTGGAACGTGGACGGTGCGGTCGACGAAG | CGTGGAACCGTGGTCAGCTGGTTACCCGTCTGGAGGAA |
| | CCACGGGCGAGCCATTCCCTCGCGCGGATGCGATT | ATGGCGATCGACGCGGGCTACAAAGGTCGTGTGTTCAA |
| | GTTCTCGAAAAGCTCGAAGGTTTCATCCCGGATGC | GGTTCATCCGGCGGGTACCAGCCAGGTTTGCAGCCGTT |
| | CGAAAAAGAGCGCGGGATCAACCGCAGTCTTGCCG | GCGGTGCGCTGGGTCGTCGTTATAGCATTACCCGTGAT |
| | CATGGAACCGCGGCCAACTGGTAACACGCCTCGAG | AACGCGGCGCACACCCCGGACATCCGTTTCGGCTGGGT |
| | GAGATGGCGATTGACGCCGGCTACAAAGGTCGTGT | GGAAAAACTGTTTGCGTGCCCGTGCGGTTACCGTGCGA |
| | TTTCAAGGTCCATCCGGCCGGTACGTCGCAGGTGT | ACAGCGATCACAACGCGAGCGTTAACCTGCAGCGTAAA |
| | GTTCCCGTTGCGGCGCGCTCGGACGGCGTTACTCA | TTCCAAATGGGTGACGAGGCGGTGAAGGCGTTTAGCAG |
| | ATCACCCGCGACAATGCCGCGCACACGCCCGACAT | CTGGCGTAACCAGACCGAAGCGCAGCGTCAACATGCGC |
| | TCGCTTTGGCTGGGTCGAAAAGCTCTTTGCGTGCCC | TGGAGAGCCTGGATGCGAGCCTGCGTGATGGCCTGCGT |
| | GTGCGGTTATCGCGCCAACTCCGACCACAATGCCT | AAGATGCATGGTCTGCCGTTCCCGCCGCTGGACAACCC |
| | CCGTCAACCTTCAGCGGAAATTCCAGATGGGCGAC | GTTTTAG (SEQ ID NO: 60) |
| | GAGGCAGTAAAGGCGTTCTCCTCGTGGCGAAATCA | |
| | AACCGAAGCCCAACGGCAACACGCCCTTGAGAGCT | |
| | TGGACGCCTCGCTCCGGGATGGCTTGCGGAAAATG | |
| | CACGGGTTGCCGTTTCCGCCTCTTGATAATCCCTTT | |
| | TGA (SEQ ID NO: 59) | |

In some embodiments, the Type V endonuclease of the disclosure is catalytically active.

In some embodiments, the Type V endonuclease of the disclosure is catalytically dead, e.g. by introducing mutations in one or more of the RuvC domains.

In some embodiments, the Type V endonuclease of the disclosure targets double stranded DNA, and is a Type V nickase.

The Type V endonucleases of the disclosure can be modified to include an aptamer.

The Type V endonuclease of the disclosure can be further fused to domains, e.g. catalytic domains to produce dual action Cas proteins. In some embodiments, a Type V endonuclease is further fused to a base editor.

### Collateral Activity of Class 2 Type V CRISPR-Cas RNA-Guided Endonucleases

In addition to the ability to cleave a target sequence in a single or double stranded targeted DNA, the Type V endonucleases of the disclosure also possess collateral (trans-cleavage activity), i.e. the ability to promiscuously cleave non-targeted DNA or RNA once activated by detection of a target DNA. Without being bound to any theory or mechanism, generally once a Type V endonuclease of the disclosure is activated by a gRNA, which occurs when a sample includes a target sequence to which the gRNA hybridizes (i.e., the sample includes the targeted DNA), the Type V endonuclease can become a nuclease that promiscuously cleaves oligonucleotides (e.g. ssDNAs, RNAs, chimeric RNA/DNAs) not comprising the target sequence of the gRNA (non-target oligonucleotides, to which the guide sequence of the gRNA does not hybridize). Thus, when the targeted DNA (double or single stranded) is present in the sample (e.g., in some embodiments above a threshold amount), the result can be cleavage of single stranded oligonucleotides (e.g. ssDNAs, ssRNAs, single stranded chimeric RNA/DNAs) in the sample, which can be detected using any convenient detection method (e.g., using a labeled detector DNA, RNA, or DNA/RNA chimera).

Accordingly, provided herein are methods and compositions for detecting a target DNA (dsDNA or ssDNA) in a sample. Also provided are methods and compositions for cleaving non-target oligonucleotides, which can be utilized detectors. These embodiments are described in further detail below.

gRNAs for Class 2 Type V CRISPR-Cas RNA-Guided Endonucleases

The present disclosure provides DNA-targeting guide RNAs that direct the activities of the novel Type V endonucleases of the disclosure to a specific target sequence within a target DNA. These DNA-targeting RNAs are referred to herein as “gRNAs” or “gRNAs”. In some embodiments, a Type V gRNA can comprise a single segment comprising both a spacer (DNA-targeting sequence) and a Cas “protein-binding sequence” together referred to as a crRNA (e.g. Cas 12a-endonuclease). In other embodiments, a Type V gRNA can comprise a first segment (also referred to herein as a “targeter-RNA”, a “DNA-targeting segment” or a “DNA-targeting sequence”) and a second segment (also referred to herein as a “activator-RNA”, a “activator-RNA” or a “protein-binding sequence”). Also provided herein are nucleotide sequences encoding the Type V gRNAs of the disclosure.

i. crRNA/Spacer Sequences for Single-RNA Guided Systems

Certain Type V endonucleases of the disclosure can be guided by a single crRNA (single-RNA guided systems). A prototypic CRISPR-Cas protein of this class includes Cas12a. The crRNA of the Type V single RNA system guides of the disclosure comprises a nucleotide sequence that is complementary to a sequence in a target DNA (DNA-targeting sequence or spacer). A prototypic CRISPR-Cas protein of this class includes Cas12a.

The crRNA portion of the Type V gRNAs of the disclosure can have a length of from about 25-50 nt. In some embodiments, the length can be about 40-43 nt.

The DNA-targeting spacer sequence of a Type V gRNA generally interacts with a target DNA in a sequence-specific manner via hybridization (i.e., base pairing). As such, the nucleotide sequence of the DNA-targeting sequence may vary and determines the location within the target DNA that the gRNA and the target DNA will interact. The DNA-targeting sequence of a subject Type V gRNA can be modified (e.g., by genetic engineering) to hybridize to a desired sequence within a target DNA.

The DNA-targeting sequence of a subject Type V gRNA can have a length of from about 8 nucleotides to about 30 nucleotides. For example, the length can be 20-23 nucleotides.

The percent complementarity between the DNA-targeting spacer sequence of the crRNA and the target sequence of the target DNA can be at least 60% (e.g., at least 65%, at least 70%, at least 75%, at least 80%, at least 85%, at least 90%, at least 95%, at least 97%, at least 98%, at least 99%, or 100%). In some embodiments, the percent complementarity between the DNA-targeting sequence of the crRNA-RNA and the target sequence of the target DNA is 100% over the 1-23 contiguous 5′-most nucleotides of the target sequence of the complementary strand of the target DNA. In some embodiments, the percent complementarity between the DNA-targeting sequence of the crRNA and the target sequence of the target DNA is at least 60% over about 1-23 contiguous nucleotides. In some embodiments, the percent complementarity between the DNA-targeting sequence of the crRNA and the target sequence of the target DNA is 100% over the 1-23 contiguous 5′-most nucleotides of the target sequence of the complementary strand of the target DNA and as low as 0% over the remainder. In such a case, the DNA-targeting sequence can be considered to be 1-23 nucleotides in length.

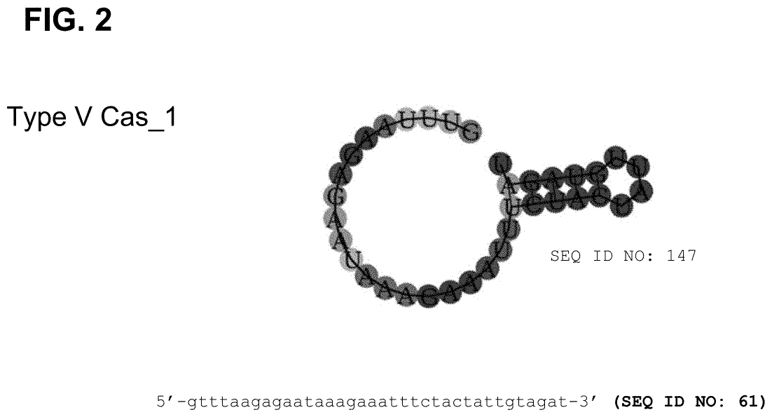

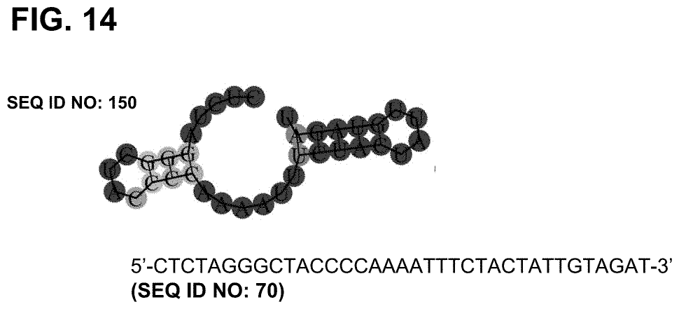

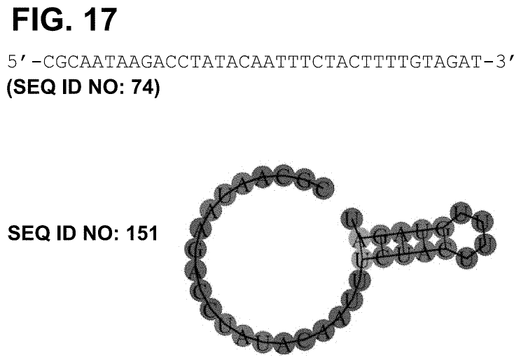

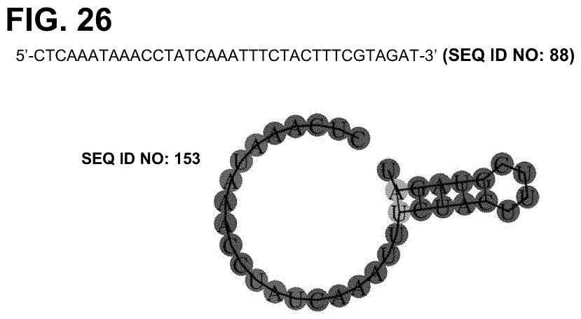

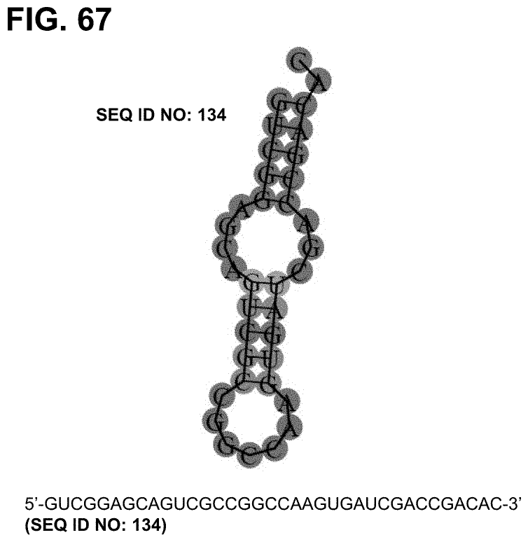

Generally, a naturally unprocessed pre-crRNA of Type V (single-RNA guided system) comprises a direct repeat and an adjacent spacer (the portion of the crRNA that allows for targeting to a DNA molecule). In some embodiments, direct repeats (partial sequence or entire sequence) from unprocessed pre-crRNA are included into the Type V gRNAs of the disclosure, and improve gRNA stability. Exemplary direct repeat sequences include SEQ ID NO: 61, 70, 74, and 88 (DNA sequences), or SEQ ID NOS 134, 147, 150, 151 and 153 (RNA sequences). It is noted that while the exemplary sequences are provided in DNA nucleotides, it is understood that this DNA can then be transcribed into RNA. Accordingly the mature guides of disclosure may incorporate the entire or partial sequence of the exemplary direct repeat sequences provided herein; the guides may be composed of DNA nucleotides, analogous RNA nucleotides, or a combination of DNA and RNA nucleotides. Exemplary predicted secondary structures of the pre-crRNAs of the Type V endonucleases of the disclosure are presented in FIGS. 2, 14, 17, 26, and 67.

In some embodiments, the crRNAs include non-naturally occurring, engineered direct repeat sequences.

In some embodiments the spacer sequence of a Type V gRNA of the disclosure is directed to a target sequence in a mammalian organism. In some embodiments the spacer sequence is directed to a target sequence in a non-mammalian organism.

In some embodiments, the spacer sequence of a Type V gRNA of the disclosure is directed to a target sequence which is a sequence of a human. In some embodiments, the target sequence is a sequence of a non-human primate.

In some embodiments, the spacer sequence of a Type V gRNA of the disclosure is directed to a target sequence in a mammalian organism, e.g. a human or non-human primate.

In some embodiments, the spacer sequence of a Type V gRNA of the disclosure is directed to a target sequence in a bacteria.

In some embodiments, the spacer sequence of a Type V gRNA of the disclosure is directed to a target sequence in a virus.

In some embodiments, the spacer sequence of a Type V gRNA of the disclosure is directed to a target sequence in a plant.

The Type V gRNAs of the disclosure can be modified to include an aptamer.

ii. Targeter-RNA/Dual-RNA Guided systems

The above section notwithstanding, certain Type V endonucleases of the disclosure can be guided by a dual-RNA system that includes a crRNA (targeter RNA) and a auxiliary RNA; a prototypic CRISPR-Cas protein of this class includes Cas12d. Yet other Type V endonucleases of the disclosure can be guided by a dual-RNA system that includes a crRNA (targeter) and a trans-activating crRNA (tracrRNA); a prototypic CRISPR-Cas protein of this class includes Cas14. These components are discussed below.

The targeter-RNA of certain Type V endonuclease gRNAs of the disclosure comprise a nucleotide sequence that is complementary to a sequence in a target DNA (targeting sequence of the gRNA; DNA-targeting sequence; spacer sequence). The targeter-RNA can interchangeably be referred to as a crRNA. The targeter-RNA of a gRNA interacts with a target DNA in a sequence-specific manner via hybridization (i.e., base pairing). As such, the nucleotide sequence of the targeter-RNA may vary and determines the location within the target DNA that the gRNA and the target DNA will interact. The targeter-RNA of a subject gRNA can be modified (e.g., by genetic engineering) to hybridize to any desired sequence within a target DNA.

The targeter-RNA of the Type V dual-RNA guided systems can have a length of from about 12 nucleotides to about 100 nucleotides. For example, the targeter-RNA can have a length of from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, or from about 12 nt to about 19 nt. For example, the targeter-RNA can have a length of from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 19 nt to about 70 nt, from about 19 nt to about 80 nt, from about 19 nt to about 90 nt, from about 19 nt to about 100 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, from about 20 nt to about 70 nt, from about 20 nt to about 80 nt, from about 20 nt to about 90 nt, or from about 20 nt to about 100 nt.

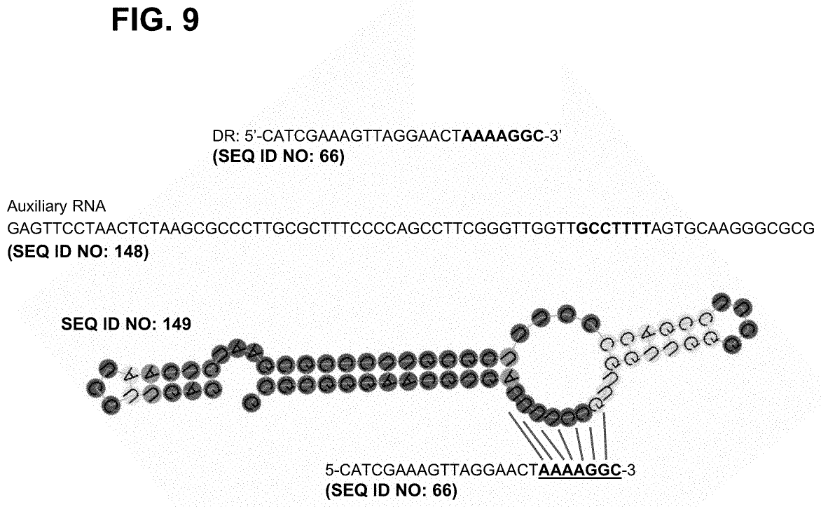

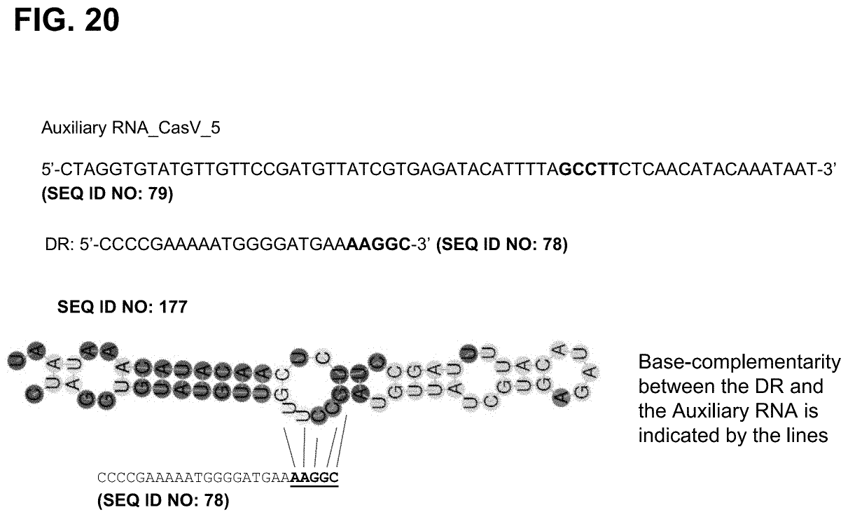

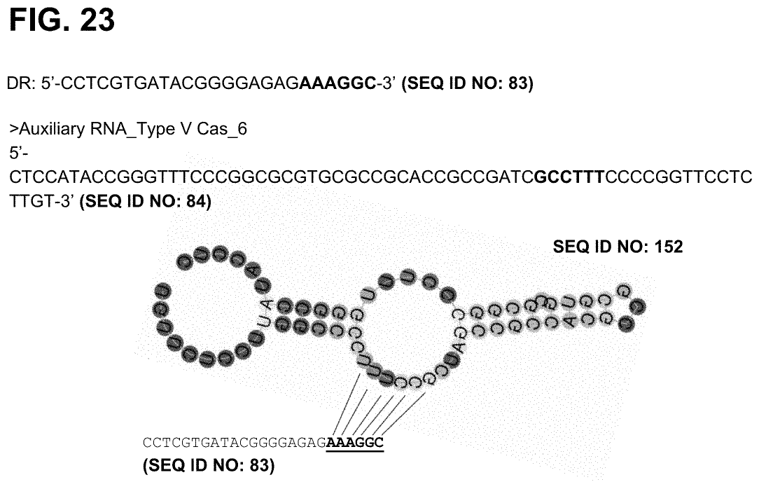

Generally, a naturally unprocessed pre-crRNA of Type V (dual RNA-guided system) comprises a direct repeat and an adjacent spacer (the portion of the crRNA that allows for targeting to a DNA molecule). In some embodiments, direct repeats (partial sequence or entire sequence) from unprocessed pre-crRNA are included into the Type V gRNAs of the disclosure, and improve gRNA stability. Exemplary direct repeat sequences include SEQ ID NO: 66, 78, and 83. It is noted that while the exemplary sequences are provided in DNA nucleotides, it is understood that this DNA can then be transcribed into RNA. Accordingly the mature guides of disclosure may incorporate the entire or partial sequence of the exemplary direct repeat sequences provided herein; the guides may be composed of DNA nucleotides, analogous RNA nucleotides, or a combination of DNA and RNA nucleotides. Exemplary predicted secondary structures of the pre-crRNAs of the Type V endonucleases (dual RNA guided systems) of the disclosure are presented in FIGS. 9, 20, and 23.

In some embodiments, the gRNAs of the disclosure include non-naturally occurring, engineered direct repeat sequences which can be incorporated into the engineered gRNAs of the disclosure.

i. Spacer Sequences/Dual-RNA Guided Systems

gRNAs of the disclosure (of the Type V dual-RNA guided systems) comprise spacer sequences, complementary to the target DNA. More specifically, the nucleotide sequence of the targeter-RNA that is complementary to a target nucleotide sequence (the DNA-targeting sequence or spacer sequence) of the target DNA can have a length at least about 12 nt. For example, the DNA-targeting sequence of the targeter-RNA that is complementary to a target sequence of the target DNA can have a length at least about 12 nt, at least about 15 nt, at least about 18 nt, at least about 19 nt, at least about 20 nt, at least about 25 nt, at least about 30 nt, at least about 35 nt or at least about 40 nt. For example, the DNA-targeting sequence of the targeter-RNA that is complementary to a target sequence of the target DNA can have a length of from about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. The nucleotide sequence (the DNA-targeting sequence) of the targeter-RNA that is complementary to a nucleotide sequence (target sequence) of the target DNA can have a length at least about 12 nt. In some embodiments, the DNA-targeting sequence of the targeter-RNA that is complementary to a target sequence of the target DNA is 20 nucleotides in length. In some embodiments, the DNA-targeting sequence of the targeter-RNA that is complementary to a target sequence of the target DNA is 19 nucleotides in length.

The percent complementarity between the spacer sequence of the targeter-RNA and the target sequence of the target DNA can be at least 60% (e.g., at least 65%, at least 70%, at least 75%, at least 80%, at least 85%, at least 90%, at least 95%, at least 97%, at least 98%, at least 99%, or 100%). In some embodiments, the percent complementarity between the DNA-targeting sequence of the targeter-RNA and the target sequence of the target DNA is 100% over the 1-25 contiguous 5′-most nucleotides of the target sequence of the complementary strand of the target DNA. In some embodiments, the percent complementarity between the DNA-targeting sequence of the targeter-RNA and the target sequence of the target DNA is at least 60% over about 1-25 contiguous nucleotides. In some embodiments, the percent complementarity between the DNA-targeting sequence of the targeter-RNA and the target sequence of the target DNA is 100% over the 1-25 contiguous 5′-most nucleotides of the target sequence of the complementary strand of the target DNA and as low as 0% over the remainder. In such a case, the DNA-targeting sequence can be considered to be 1-25 nucleotides in length.

In some embodiments the spacer sequence of a Type V dual-RNA guided system of the disclosure is directed to a target sequence in a mammalian organism. In some embodiments the spacer sequence is directed to a target sequence in a non-mammalian organism.

In some embodiments, the spacer sequence of a Type V dual-RNA guided system of the disclosure is directed to a target sequence which is a sequence of a human. In some embodiments, the target sequence is a sequence of a non-human primate.

In some embodiments the spacer sequence of a Type V dual-RNA guided system of the disclosure is directed to a target sequence selected of a therapeutic target.

In some embodiments the spacer sequence of a Type V dual-RNA guided system of the disclosure is directed to a target sequence selected of a diagnostic target—for example in such embodiments a labeled catalytically dead Type II endonuclease of the disclosure and a gRNA directed to a diagnostic target DNA is contacted with the target DNA, or a cell comprising the target DNA, or a sample comprising the target DNA.

ii. Activator-RNA/Dual-RNA Guided Systems

The activator-RNA of certain Type V gRNA of the disclosure binds with its cognate Type V endonuclease of the disclosure (e.g. Type V Cas_8 of the disclosure). The activator-RNA can interchangeably be referred to as a tracrRNA. The gRNA guides the bound Type V endonuclease to a specific nucleotide sequence within target DNA via the above described targeter-RNA. The activator-RNA of a Type V gRNA comprises two stretches of nucleotides that are complementary to one another.

iii. Dual-Molecule Type V gRNAs

As noted above, in some embodiments, provided herein are dual molecule (two-molecule) Type V gRNAs for the novel Type V endonucleases of the disclosure. Such gRNAs comprise two separate RNA molecules (tracRNA or auxiliary RNA; and the targeting RNA-crRNA). Each of the two RNA molecules of a subject double-molecule gRNA comprises a stretch of nucleotides that are complementary to one another such that the complementary nucleotides of the two RNA molecules hybridize to form the double stranded RNA duplex of the gRNA.

A dual-molecule gRNA can be designed to allow for controlled (i.e., conditional) binding of a targeter-RNA with an activator-RNA. Because a dual-molecule gRNA is not functional unless both the activator-RNA and the targeter-RNA are bound in a functional complex with Type V endonucleases of the disclosure, a dual-molecule gRNA can be inducible (e.g., drug inducible) by rendering the binding between the activator-RNA and the targeter-RNA to be inducible. As one non-limiting example, RNA aptamers can be used to regulate (i.e., control) the binding of the activator-RNA with the targeter-RNA. Accordingly, the activator-RNA and/or the targeter-RNA can comprise an RNA aptamer sequence.

The dual-molecule guide can be modified to include an aptamer.

iv. Engineered Single-Molecule Type V Endonuclease gRNAs

In some embodiments, provided herein are engineered Type V gRNAs that comprises a single-molecule gRNA (interchangeably referred to herein as a sgRNA), for the novel Type V endonucleases of the disclosure.

Accordingly provided herein is an engineered single-molecule gRNA, comprising:
a. a targeter-RNA that is capable of hybridizing with a target sequence in a target DNA; and
b. an activator-RNA that is capable of hybridizing with the targeter-RNA to form a double-stranded RNA duplex, the activator-RNA comprising a activator-RNA,
   wherein the targeter-RNA and the activator-RNA are covalently linked to one another, wherein the single-molecule gRNA is capable of forming a complex with a novel Type V endonuclease of the disclosure, and wherein hybridization of the targeter-RNA to the target sequence is capable of targeting the Type V endonuclease of the disclosure to the target DNA.

A subject engineered single-molecule gRNA comprises two segments of nucleotides (a targeter-RNA and an activator-RNA) that are complementary to one another, can be covalently linked by intervening nucleotides (“linkers” or “linker nucleotides”), and hybridize to form the double stranded RNA duplex (dsRNA duplex) of the activator-RNA, whereby resulting in a stem-loop structure. In some embodiments, the targeter-RNA and the activator-RNA are covalently linked via the 3′ end of the targeter-RNA and the 5′ end of the activator-RNA. In other embodiments, the activator-RNA is covalently linked via the 5′ end of the targeter-RNA and the 3′ end of the activator-RNA.

In some embodiments, the targeter-RNA and the activator-RNA are arranged in a 5′ to 3′ orientation.

In some embodiments, the activator-RNA and the targeter-RNA are arranged in a 5′ to 3′ orientation.

In some embodiments, the single molecule gRNA comprises one or more sequence modifications compared to a sequence of a corresponding wild type tracrRNA and/or crRNA.

In some embodiments, the targeter-RNA and the activator-RNA are covalently linked to one another via a linker.

When present, the linker of a single-molecule gRNA can have a length of from about 3 nucleotides to about 30 nucleotides. In exemplary embodiments, the linker of a single-molecule gRNA is 4, 5, 6, or 7 nt.

An exemplary single-molecule gRNA comprises two complementary stretches of nucleotides that hybridize to form a dsRNA duplex. In some embodiments, one of the two complementary stretches of nucleotides of the single-molecule gRNA (or the DNA encoding the stretch) is at least about 60% identical to one of the activator-RNA. For example, one of the two complementary stretches of nucleotides of the single-molecule gRNA (or the DNA encoding the stretch) is at least about 65% identical, at least about 70% identical, at least about 75% identical, at least about 80% identical, at least about 85% identical, at least about 90% identical, at least about 95% identical, at least about 98% identical, at least about 99% identical or 100% identical to an activator-RNA.

The activator-RNA and targeter-RNA segments can be engineered, while ensuring that the structure of the protein-binding domain of the gRNA is conserved. Thus, RNA folding structure of a naturally occurring protein-binding domain of a DNA-targeting RNA can be taken into account in order to design artificial protein-binding domains (either dual-molecule or single-molecule versions).

The activator-RNA in a single-molecule gRNA can have a length of from about 10 nucleotides to about 100 nucleotides. For example, the activator-RNA can have a length of from about 15 nucleotides (nt) to about 80 nt, from about 15 nt to about 50 nt, from about 15 nt to about 40 nt, from about 15 nt to about 30 nt or from about 15 nt to about 25 nt.

Also with regard to both the single-molecule and double-molecule gRNAs of the disclosure, the dsRNA duplex of the activator-RNA can have a length from about 6 nucleotides (nt) to about 50 bp. For example, the dsRNA duplex of the activator-RNA can have a length from about 6 nt to about 40 nt, from about 6 nt to about 30 bp, from about 6 nt to about 25 nt, from about 6 nt to about 20 nt, from about 6 nt to about 15 nt, from about 8 nt to about 40 nt, from about 8 nt to about 30 bp, from about 8 nt to about 25 nt, from about 8 nt to about 20 nt or from about 8 nt to about 15 nt. For example, the dsRNA duplex of the activator-RNA can have a length from about from about 8 nt to about 10 nt, from about 10 nt to about 15 nt, from about 15 nt to about 18 nt, from about 18 nt to about 20 nt, from about 20 nt to about 25 nt, from about 25 nt to about 30 nt, from about 30 nt to about 35 nt, from about 35 nt to about 40 nt, or from about 40 nt to about 50 nt. In some embodiments, the dsRNA duplex of the activator-RNA has a length of 8-15 base pairs. The percent complementarity between the nucleotide sequences that hybridize to form the dsRNA duplex of the activator-RNA can be at least about 60%. For example, the percent complementarity between the nucleotide sequences that hybridize to form the dsRNA duplex of the activator-RNA can be at least about 65%, at least about 70%, at least about 75%, at least about 80%, at least about 85%, at least about 90%, at least about 95%, at least about 98%, or at least about 99%. In some embodiments, the percent complementarity between the nucleotide sequences that hybridize to form the dsRNA duplex of the activator-RNA is 100%.

In some embodiments, the spacer sequence of a Type V gRNA (whether it is a single molecule gRNA or a dual molecule gRNA) of the disclosure is directed to a target sequence in a mammalian organism, e.g. a human or non-human primate. In some embodiments, the spacer sequence of a Type V gRNA of the disclosure is directed to a target sequence in a bacteria.

In some embodiments, the spacer sequence of a Type V gRNA of the disclosure is directed to a target sequence in a virus. In some embodiments, the spacer sequence of a Type V gRNA of the disclosure is directed to a target sequence in a plant.

In some embodiments, the single-molecule Type V gRNAs of the disclosure can be modified to include an aptamer.

v. gRNA Arrays

In some embodiments, the Type V gRNAs of the disclosure can be provided as gRNA arrays.

Such gRNA arrays of the disclosure include more than one gRNA arrayed in tandem, and can be processed into two or more individual gRNAs. Thus, in some embodiments a precursor Type V gRNA array comprises two or more (e.g., 3 or more, 4 or more, 5 or more, 2, 3, 4, or 5) gRNAs (e.g., arrayed in tandem as precursor molecules). In some embodiments, two or more gRNAs can be present on an array (a precursor gRNA array). A Type V endonuclease of the disclosure can cleave the precursor gRNA array into individual gRNAs.

In some embodiments a Type V gRNA array includes 2 or more gRNAs (e.g., 3 or more, 4 or more, 5 or more, 6 or more, or 7 or more, gRNAs). The gRNAs of a given array can target (i.e., can include guide sequences that hybridize to) different target sites of the same target DNA. In some embodiments, two or more gRNAs of a precursor gRNA array have the same guide sequence. In some embodiments, the precursor gRNA array comprises two or more gRNAs that target different target sites within the same target DNA. In some embodiments, the precursor gRNA array comprises two or more gRNAs that target different target DNAs.

### II. Class 2 Type VI CRISPR-Cas RNA-Guided Systems

Provided herein are novel Class 2 Type VI CRISPR-Cas RNA-guided proteins and their guide RNAs (a “guide RNA” is interchangeably referred to herein as “gRNA”), constituting the Class 2 Type VI CRISPR-Cas RNA-guided systems of the disclosure.

Accordingly, provided herein are systems comprising (a) Type VI endonuclease, or a nucleic acid encoding the Type VI endonuclease; and (b) a Type VI gRNA, or a nucleic acid encoding the Type VI gRNA, wherein the gRNA and the Type VI endonuclease do not naturally occur together, wherein the gRNA is capable of hybridizing to a target sequence in a target single stranded RNA, and the gRNA is capable of forming a complex with the Type VI endonuclease.

The components of the system described in turn below.

### Type VI CRISPR-Cas RNA-Guided Endonucleases

Also provided herein are novel Type VI CRISPR-Cas RNA-guided endonucleases. In some embodiments, these endonucleases may share certain structural, sequence, and/or functional similarities with any one of the subtypes of Cas13 (e.g. Cas13a, Cas13b). Such Type VI endonucleases are useful for RNA targeting and modification. Type VI targets ssRNA and requires a protospacer flanking sequence (PFS) instead of the PAM required for dsDNA unwinding, e.g. for Type II and Type V endonucleases.

Without being bound to any theory or mechanism, a Type VI CRISPR-Cas RNA-guided endonucleases of the disclosure comprise two HEPN motifs, generally of the motif E RXXXXH (SEQ ID NO: 93), also referred to as E . . . R-X4-H (SEQ ID NO: 93). The distance between the E residue and the R-X4-H (SEQ ID NO: 93) can be of any length.

In some embodiments a Type VI CRISPR-Cas RNA-guided endonuclease of the disclosure comprises any one of the HEPN sequences of Table 4, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type VI CRISPR-Cas RNA-guided endonuclease of the disclosure comprises any two of the HEPN sequences of Table 4, or sequences comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type VI CRISPR-Cas RNA-guided endonuclease of the disclosure comprises a HEPN motif selected from the group consisting of SEQ ID NO: 94, SEQ ID NO: 95, SEQ ID NO: 97, SEQ ID NO: 99, SEQ ID NO: 100, SEQ ID NO: 102, SEQ ID NO: 104, SEQ ID NO: 105, SEQ ID NO: 107, SEQ ID NO: 108, SEQ ID NO: 110, SEQ ID NO: 111, SEQ ID NO: 113, and SEQ ID NO: 197, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type VI CRISPR-Cas RNA-guided endonuclease of the disclosure comprises a first HEPN motif selected from the group consisting of SEQ ID NO: 94, SEQ ID NO: 95, SEQ ID NO: 97, SEQ ID NO: 99, SEQ ID NO: 100, SEQ ID NO: 102, SEQ ID NO: 104, SEQ ID NO: 105, SEQ ID NO: 107, SEQ ID NO: 108, SEQ ID NO: 110, SEQ ID NO: 111, SEQ ID NO: 113, and SEQ ID NO: 197, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; and comprises a second HEPN motif selected from the group consisting of SEQ ID NO: 94, SEQ ID NO: 95, SEQ ID NO: 97, SEQ ID NO: 99, SEQ ID NO: 100, SEQ ID NO: 102, SEQ ID NO: 104, SEQ ID NO: 105, SEQ ID NO: 107, SEQ ID NO: 108, SEQ ID NO: 110, SEQ ID NO: 111, SEQ ID NO: 113, and SEQ ID NO: 197, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type VI CRISPR-Cas RNA-guided endonuclease of the disclosure comprises a first HEPN motif comprising the amino acid sequence of SEQ ID NO: 94, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; and comprises a second HEPN motif comprising the amino acid sequence of SEQ ID NO: 95 or SEQ ID NO: 197, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type VI CRISPR-Cas RNA-guided endonuclease of the disclosure comprises a first HEPN motif comprising the amino acid sequence of SEQ ID NO: 97, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; and comprises a second HEPN motif comprising the amino acid sequence of SEQ ID NO: 95 or SEQ ID NO: 197, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type VI CRISPR-Cas RNA-guided endonuclease of the disclosure comprises a first HEPN motif comprising the amino acid sequence of SEQ ID NO: 99, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; and comprises a second HEPN motif comprising the amino acid sequence of SEQ ID NO: 100, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type VI CRISPR-Cas RNA-guided endonuclease of the disclosure comprises a first HEPN motif comprising the amino acid sequence of SEQ ID NO: 95 or SEQ ID NO: 197, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; and comprises a second HEPN motif comprising the amino acid sequence of SEQ ID NO: 102, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type VI CRISPR-Cas RNA-guided endonuclease of the disclosure comprises a first HEPN motif comprising the amino acid sequence of SEQ ID NO: 104, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; and comprises a second HEPN motif comprising the amino acid sequence of SEQ ID NO: 105, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type VI CRISPR-Cas RNA-guided endonuclease of the disclosure comprises a first HEPN motif comprising the amino acid sequence of SEQ ID NO: 107, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; and comprises a second HEPN motif comprising the amino acid sequence of SEQ ID NO: 108, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type VI CRISPR-Cas RNA-guided endonuclease of the disclosure comprises a first HEPN motif comprising the amino acid sequence of SEQ ID NO: 110 or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; and comprises a second HEPN motif comprising the amino acid sequence of SEQ ID NO: 111, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type VI CRISPR-Cas RNA-guided endonuclease of the disclosure comprises a first HEPN motif comprising the amino acid sequence of SEQ ID NO: 99, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; and comprises a second HEPN motif comprising the amino acid sequence of SEQ ID NO: 113, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

Table 4 provided exemplary HEPN sequences of the Type VI endonucleases of the disclosure.

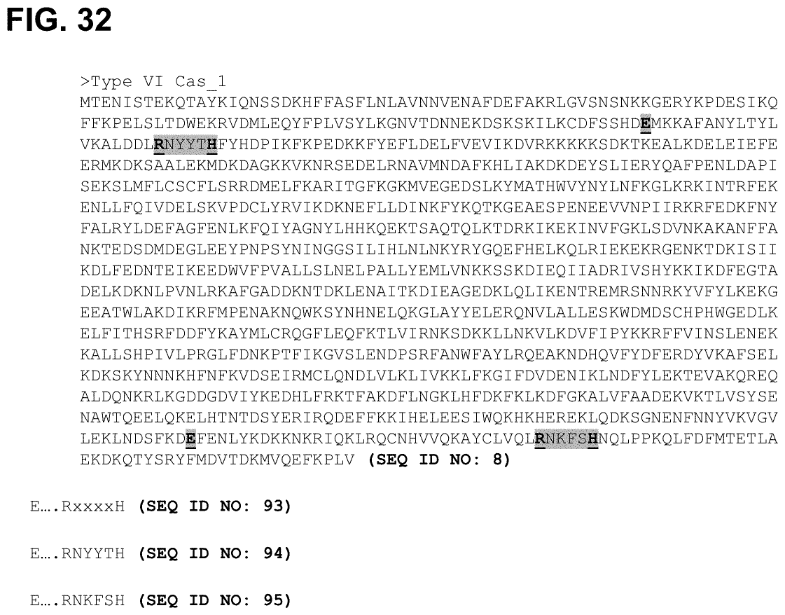

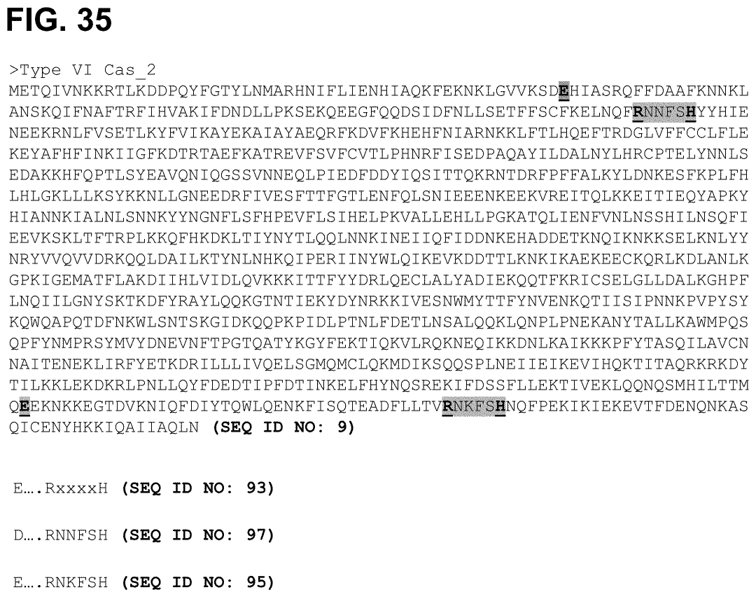

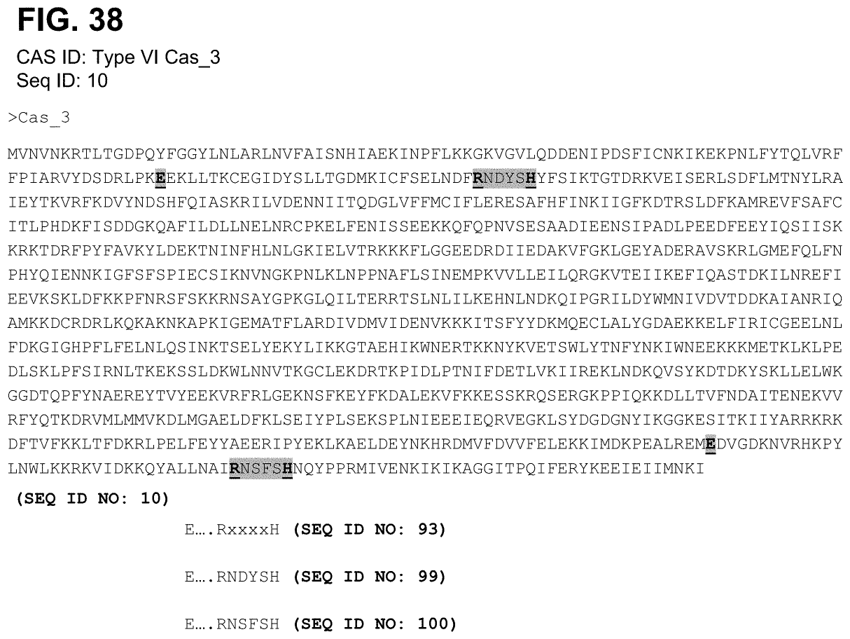

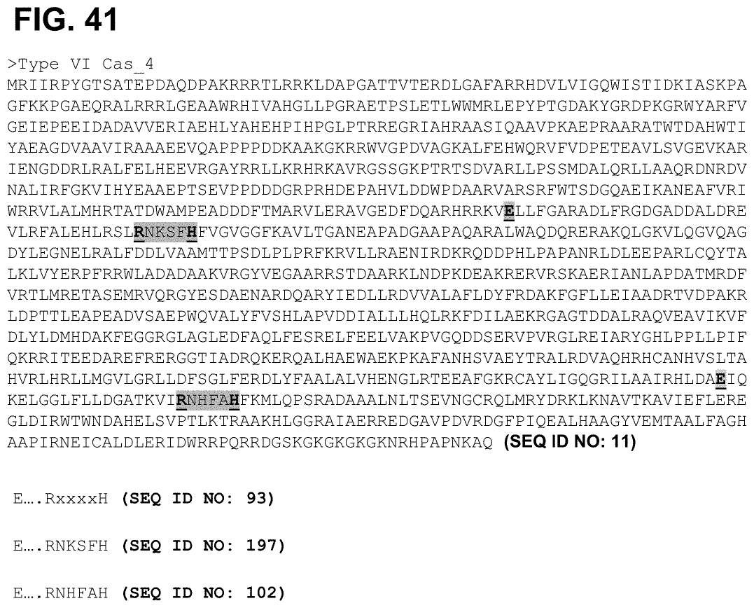

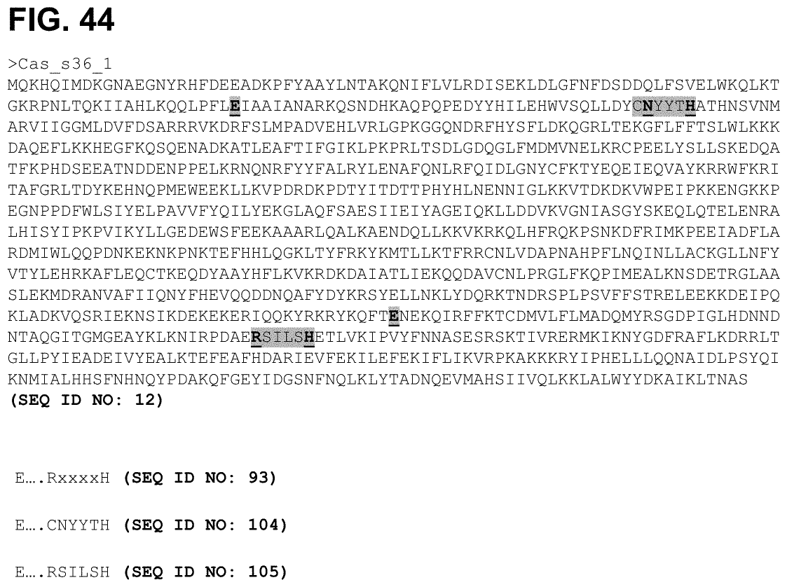

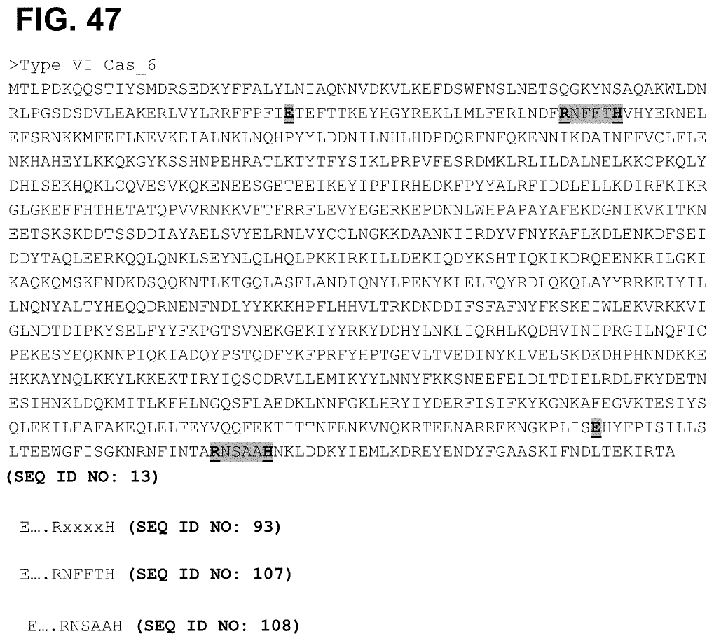

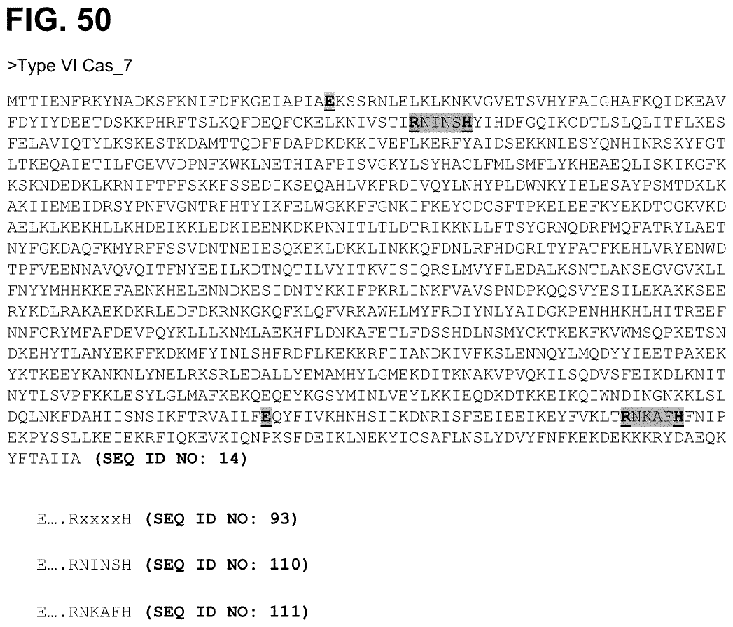

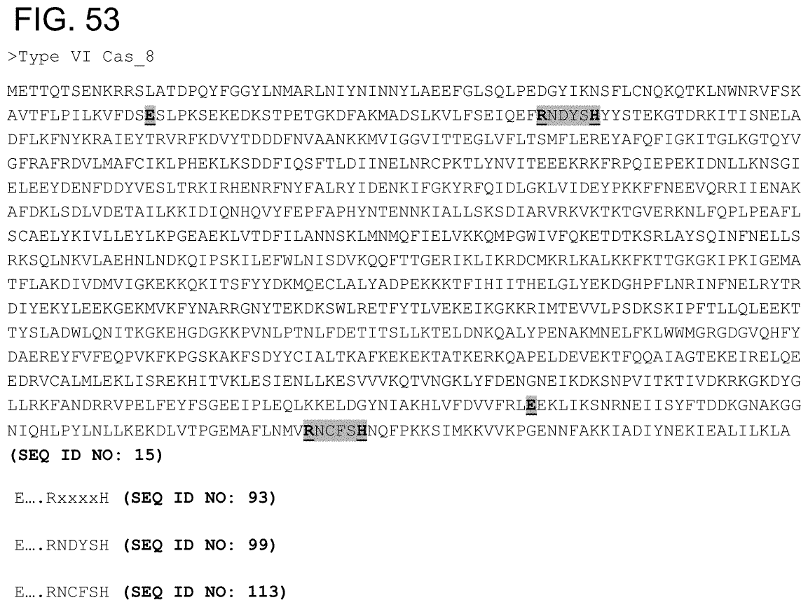

TABLE 4

| SEQ | | | |
|---|---|---|---|
| ID | Exemplary | | |
| NO: | Figure | MOTIF | SEQUENCE |
| 94 | Fig. 32 | HEPN motif | E... . RNYYTH |
| 95 | FIG. 32 | HEPN motif | E... . RNEFSH |
| 97 | FIG. 35 | HEPN motif | E... . RNNFSH |
| 99 | FIG. 38 | HEPN motif | E... . RNDYSH |
| 100 | FIG. 38 | HEPN motif | E... . RNSFSH |
| 102 | FIG. 41 | HEPN motif | E... . RNHFAH |
| 104 | FIG. 44 | HEPN motif | E... . CNYYTH |
| 105 | FIG. 44 | HEPN motif | E... . RSILSH |
| 107 | FIG. 47 | HEPN motif | E... . RNFFTH |
| 108 | FIG. 47 | HEPN motif | E... . RNSAAH |
| 110 | FIG. 50 | HEPN motif | E... . RNINSH |
| 111 | FIG. 50 | HEPN motif | E... . RNKAFH |
| 113 | FIG. 53 | HEPN motif | E... . RNCFSH |

Table 5 provides exemplary amino acid sequences for certain Type VI sequences of the disclosure. Genes were identified from metagenomic samples. Scripts were run on the sequences, designed to find CRISPR sequences and accompanying genes encoding proteins showing homology with reported Cas enzymes. Comparative BlastP analyses were performed against sequences deposited in databases (NCBI, LENS), discarding those candidates showing Id % >50 with deposited proteins. Presence of specific domains (e.g. RuvC, HEPN) and catalytic motifs were determined (CD-search, phmmer, UNIPROT).

TABLE 5

| | FIGURE | |
|---|---|---|
| | AND SEQ | |
| NAME | ID | AMINO ACID SEQUENCE |
| Type VI | FIG. 32 | MTENISTEKQTAYKIQNSSDKHFFASFLNLAVNNVENAFDEFAKRLGVSNS |
| Cas_1 | SEQ ID NO: | NKKGERYKPDESIKQFFKPELSLTDWEKRVDMLEQYFPLVSYLKGNVTDN |
| | 8 | NEKDSKSKILKCDFSSHDEMKKAFANYLTYLVKALDDLRNYYTHFYHDPI |
| | | KFKPEDKKFYEFLDELFVEVIKDVRKKKKKSDKTKEALKDELEIEFEERMK |
| | | DKSAALEKMDKDAGKKVKNRSEDELRNAVMNDAFKHLIAKDKDEYSLIE |
| | | RYQAFPENLDAPISEKSLMFLCSCFLSRRDMELFKARITGFKGKMVEGEDSL |
| | | KYMATHWVYNYLNFKGLKRKINTRFEKENLLFQIVDELSKVPDCLYRVIK |
| | | DKNEFLLDINKFYKQTKGEAESPENEEVVNPIIRKRFEDKFNYFALRYLDEF |
| | | AGFENLKFQIYAGNYLHHKQEKTSAQTQLKTDRKIKEKINVFGKLSDVNKA |
| | | KANFFANKTEDSDMDEGLEEYPNPSYNINGGSILIHLNLNKYRYGQEFHEL |
| | | KQLRIEKEKRGENKTDKISIIKDLFEDNTEIKEEDWVFPVALLSLNELPALLY |
| | | EMLVNKKSSKDIEQIIADRIVSHYKKIKDFEGTADELKDKNLPVNLRKAFGA |
| | | DDKNTDKLENAITKDIEAGEDKLQLIKENTREMRSNNRKYVFYLKEKGEEA |
| | | TWLAKDIKRFMPENAKNQWKSYNHNELQKGLAYYELERQNVLALLESKW |
| | | DMDSCHPHWGEDLKELFITHSRFDDFYKAYMLCRQGFLEQFKTLVIRNKS |
| | | DKKLLNKVLKDVFIPYKKRFFVINSLENEKKALLSHPIVLPRGLFDNKPTFIK |
| | | GVSLENDPSRFANWFAYLRQEAKNDHQVFYDFERDYVKAFSELKDKSKY |
| | | NNNKHFNFKVDSEIRMCLQNDLVLKLIVKKLFKGIFDVDENIKLNDFYLEK |
| | | TEVAKQREQALDQNKRLKGDDGDVIYKEDHLFRKTFAKDFLNGKLHFDKF |
| | | KLKDFGKALVFAADEKVKTLVSYSENAWTQEELQKELHTNTDSYERIRQD |
| | | EFFKKIHELEESIWQKHKHEREKLQDKSGNENFNNYVKVGVLEKLNDSFK |
| | | DEFENLYKDKKNKRIQKLRQCNHVVQKAYCLVQLRNKFSHNQLPPKQLFD |
| | | FMTETLAEKDKQTYSRYFMDVTDKMVQEFKPLV |
| Type VI | FIG. 35 | METQIVNKKRTLKDDPQYFGTYLNMARHNIFLIENHIAQKFEKNKLGVVKS |
| Cas_2 | SEQ ID NO: | DEHIASRQFFDAAFKNNKLANSKQIFNAFTRFIHVAKIFDNDLLPKSEKQEE |
| | 9 | GFQQDSIDFNLLSETFFSCFKELNQFRNNFSHYYHIENEEKRNLFVSETLKYF |
| | | VIKAYEKAIAYAEQRFKDVFKHEHFNIARNKKLFTLHQEFTRDGLVFFCCL |
| | | FLEKEYAFHFINKIIGFKDTRTAEFKATREVFSVFCVTLPHNRFISEDPAQAYI |
| | | LDALNYLHRCPTELYNNLSEDAKKHFQPTLSYEAVQNIQGSSVNNEQLPIE |
| | | DFDDYIQSITTQKRNTDRFPFFALKYLDNKESFKPLFHLHLGKLLLKSYKKN |
| | | LLGNEEDRFIVESFTTFGTLENFQLSNIEEENKEEKVREITQLKKEITIEQYAP |
| | | KYHIANNKIALNLSNNKYYNGNFLSFHPEVFLSIHELPKVALLEHLLPGKAT |
| | | QLIENFVNLNSSHILNSQFIEEVKSKLTFTRPLKKQFHKDKLTIYNYTLQQLN |
| | | NKINEIIQFIDDNKEHADDETKNQIKNKKSELKNLYYNRYVVQVVDRKQQL |
| | | DAILKTYNLNHKQIPERIINYWLQIKEVKDDTTLKNKIKAEKEECKQRLKDL |
| | | ANLKGPKIGEMATFLAKDIIHLVIDLQVKKKITTFYYDRLQECLALYADIEK |
| | | QQTFKRICSELGLLDALKGHPFLNQIILGNYSKTKDFYRAYLQQKGTNTIEK |
| | | YDYNRKKIVESNWMYTTFYNVENKQTIISIPNNKPVPYSYKQWQAPQTDF |
| | | NKWLSNTSKGIDKQQPKPIDLPTNLFDETLNSALQQKLQNPLPNEKANYTA |
| | | LLKAWMPQSQPFYNMPRSYMVYDNEVNFTPGTQATYKGYFEKTIQKVLR |
| | | QKNEQIKKDNLKAIKKKPFYTASQILAVCNNAITENEKLIRFYETKDRILLLI |
| | | VQELSGMQMCLQKMDIKSQQSPLNEHEIKEVIHQKTITAQRKRKDYTILKK |
| | | LEKDKRLPNLLQYFDEDTIPFDTINKELFHYNQSREKIFDSSFLLEKTIVEKL |
| | | QQNQSMHILTTMQEEKNKKEGTDVKNIQFDIYTQWLQENKFISQTEADFLL |
| | | TVRNKFSHNQFPEKIKIEKEVTFDENQNKASQICENYHKKIQAIIAQLN |
| Type VI | FIG. 38 | MVNVNKRTLTGDPQYFGGYLNLARLNVFAISNHIAEKINPFLKKGKVGVL |
| Cas_3 | SEQ ID NO: | QDDENIPDSFICNKIKEKPNLFYTQLVRFFPIARVYDSDRLPKEEKLLTKCEG |
| | 10 | IDYSLLTGDMKICFSELNDFRNDYSHYFSIKTGTDRKVEISERLSDFLMTNY |
| | | LRAIEYTKVRFKDVYNDSHFQIASKRILVDENNIITQDGLVFFMCIFLERESA |
| | | FHFINKIIGFKDTRSLDFKAMREVFSAFCITLPHDKFISDDGKQAFILDLLNEL |
| | | NRCPKELFENISSEEKKQFQPNVSESAADIEENSIPADLPEEDFEEYIQSIISKK |
| | | RKTDRFPYFAVKYLDEKTNINFHLNLGKIELVTRKKKFLGGEEDRDIIEDAK |
| | | VFGKLGEYADERAVSKRLGMEFQLFNPHYQIENNKIGFSFSPIECSIKNVNG |
| | | KPNLKLNPPNAFLSINEMPKVVLLEILQRGKVTEIIKEFIQASTDKILNREFIE |
| | | EVKSKLDFKKPFNRSFSKKRNSAYGPKGLQILTERRTSLNLILKEHNLNDKQ |
| | | IPGRILDYWMNIVDVTDDKAIANRIQAMKKDCRDRLKQKAKNKAPKIGEM |
| | | ATFLARDIVDMVIDENVKKKITSFYYDKMQECLALYGDAEKKELFIRICGE |
| | | ELNLFDKGIGHPFLFELNLQSINKTSELYEKYLIKKGTAEHIKWNERTKKNY |
| | | KVETSWLYTNFYNKIWNEEKKKMETKLKLPEDLSKLPFSIRNLTKEKSSLD |
| | | KWLNNVTKGCLEKDRTKPIDLPTNIFDETLVKIIREKLNDKQVSYKDTDKY |
| | | SKLLELWKGGDTQPFYNAEREYTVYEEKVRFRLGEKNSFKEYFKDALEKV |
| | | FKKESSKRQSERGKPPIQKKDLLTVFNDAITENEKVVRFYQTKDRVMLMM |
| | | VKDLMGAELDFKLSEIYPLSEKSPLNIEEEIEQRVEGKLSYDGDGNYIKGGK |
| | | ESITKIIYARRKRKDFTVFKKLTFDKRLPELFEYYAEERIPYEKLKAELDEYN |
| | | KHRDMVFDVVFELEKKIMDKPEALREMEDVGDKNVRHKPYLNWLKKRK |
| | | VIDKKQYALLNAIRNSFSHNQYPPRMIVENKIKIKAGGITPQIFERYKEEIEII |
| | | MNKI |
| Type VI | FIG. 41 | MRIIRPYGTSATEPDAQDPAKRRRTLRRKLDAPGATTVTERDLGAFARRHD |
| Cas_3 | SEQ ID NO: | VLVIGQWISTIDKIASKPAGFKKPGAEQRALRRRLGEAAWRHIVAHGLLPG |
| | 11 | RAETPSLETLWWMRLEPYPTGDAKYGRDPKGRWYARFVGEIEPEEIDADA |
| | | VVERIAEHLYAHEHPIHPGLPTRREGRIAHRAASIQAAVPKAEPRAARATW |
| | | TDAHWTIYAEAGDVAAVIRAAAEEVQAPPPPDDKAAKGKRRWVGPDVAG |
| | | KALFEHWQRVFVDPETEAVLSVGEVKARIENGDDRLRALFELHEEVRGAY |
| | | RRLLKRHRKAVRGSSGKPTRTSDVARLLPSSMDALQRLLAAQRDNRDVNA |
| | | LIRFGKVIHYEAAEPTSEVPPDDDGRPRHDEPAHVLDDWPDAARVARSRF |
| | | WTSDGQAEIKANEAFVRIWRRVLALMHRTATDWAMPEADDDFTMARVLE |
| | | RAVGEDFDQARHRRKVELLFGARADLFRGDGADDALDREVLRFALEHLRS |
| | | LRNKSFHFVGVGGFKAVLTGANEAPADGAAPAQARALWAQDQRERAKQL |
| | | GKVLQGVQAGDYLEGNELRALFDDLVAAMTTPSDLPLPRFKRVLLRAENI |
| | | RDKRQDDPHLPAPANRLDLEEPARLCQYTALKLVYERPFRRWLADADAA |
| | | KVRGYVEGAARRSTDAARKLNDPKDEAKRERVRSKAERIANLAPDATMR |
| | | DFVRTLMRETASEMRVQRGYESDAENARDQARYIEDLLRDVVALAFLDYF |
| | | RDAKFGFLLEIAADRTVDPAKRLDPTTLEAPEADVSAEPWQVALYFVSHLA |
| | | PVDDIALLLHQLRKFDILAEKRGAGTDDALRAQVEAVIKVFDLYLDMHDA |
| | | KFEGGRGLAGLEDFAQLFESRELFEELVAKPVGQDDSERVPVRGLREIARY |
| | | GHLPPLLPIFQKRRITEEDAREFRERGGTIADRQKERQALHAEWAEKPKAFA |
| | | NHSVAEYTRALRDVAQHRHCANHVSLTAHVRLHRLLMGVLGRLLDFSGL |
| | | FERDLYFAALALVHENGLRTEEAFGKRCAYLIGQGRILAAIRHLDAEIQKEL |
| | | GGLFLLDGATKVIRNHFAHFKMLQPSRADAAALNLTSEVNGCRQLMRYDR |
| | | KLKNAVTKAVIEFLEREGLDIRWTWNDAHELSVPTLKTRAAKHLGGRAIA |
| | | ERREDGAVPDVRDGFPIQEALHAAGYVEMTAALFAGHAAPIRNEICALDLE |
| | | REDWRRPQRRDGSKGKGKGKGKNRHPAPNKAQ |
| Type VI | FIG. 44 | MQKHQIMDKGNAEGNYRHFDEEADKPFYAAYLNTAKQNIFLVLRDISEKL |
| Cas_5 | SEQ ID NO: | DLGFNFDSDDQLFSVELWKQLKTGKRPNLTQKIIAHLKQQLPFLEIAAIANA |
| | 12 | RKQSNDHKAQPQPEDYYHILEHWVSQLLDYCNYYTHATHNSVNMARVIIG |
| | | GMLDVFDSARRRVKDRFSLMPADVEHLVRLGPKGGQNDRFHYSFLDKQG |
| | | RLTEKGFLFFTSLWLKKKDAQEFLKKHEGFKQSQENADKATLEAFTIFGIKL |
| | | PKPRLTSDLGDQGLFMDMVNELKRCPEELYSLLSKEDQATFKPHDSEEATN |
| | | DDENPPELKRNQNRFYYFALRYLENAFQNLRFQIDLGNYCFKTYEQEIEQV |
| | | AYKRRWFKRITAFGRLTDYKEHNQPMEWEEKLLKVPDRDKPDTYITDTTP |
| | | HYHLNENNIGLKKVTDKDKVWPEIPKKENGKKPEGNPPDFWLSIYELPAVV |
| | | FYQILYEKGLAQFSAESIIEIYAGEIQKLLDDVKVGNIASGYSKEQLQTELEN |
| | | RALHISYIPKPVIKYLLGEDEWSFEEKAAARLQALKAENDQLLKKVKRKQL |
| | | HFRQKPSNKDFRIMKPEEIADFLARDMIWLQQPDNKEKNKPNKTEFHHLQ |
| | | GKLTYFRKYKMTLLKTFRRCNLVDAPNAHPFLNQINLLACKGLLNFYVTY |
| | | LEHRKAFLEQCTKEQDYAAYHFLKVKRDKDAIATLIEKQQDAVCNLPRGL |
| | | FKQPIMEALKNSDETRGLAASLEKMDRANVAFIIQNYFHEVQQDDNQAFY |
| | | DYKRSYELLNKLYDQRKTNDRSPLPSVFFSTRELEEKKDEIPQKLADKVQS |
| | | RIEKNSIKDEKEKERIQQKYRKRYKQFTENEKQIRFFKTCDMVLFLMADQM |
| | | YRSGDPIGLHDNNDNTAQGITGMGEAYKLKNIRPDAERSILSHETLVKIPVY |
| | | FNNASESRSKTIVRERMKIKNYGDFRAFLKDRRLTGLLPYIEADEIVYEALK |
| | | TEFEAFHDARIEVFEKILEFEKIFLIKVRPKAKKKRYIPHELLLQQNAIDLPSY |
| | | QIKNMIALHHSFNHNQYPDAKQFGEYIDGSNFNQLKLYTADNQEVMAHSII |
| | | VQLKKLALWYYDKAIKLTNAS |
| Type VI | FIG. 47 | MTLPDKQQSTIYSMDRSEDKYFFALYLNIAQNNVDKVLKEFDSWFNSLNE |
| Cas_6 | SEQ ID NO: | TSQGKYNSAQAKWLDNRLPGSDSDVLEAKERLVYLRRFFPFIETEFTTKEY |
| | 13 | HGYREKLLMLFERLNDFRNFFTHVHYERNELEFSRNKKMFEFLNEVKEIAL |
| | | NKLNQHPYYLDDNILNHLHDPDQRFNFQKENNIKDAINFFVCLFLENKHAH |
| | | EYLKKQKGYKSSHNPEHRATLKTYTFYSIKLPRPVFESRDMKLRLILDALN |
| | | ELKKCPKQLYDHLSEKHQKLCQVESVKQKENEESGETEEIKEYIPFIRHEDK |
| | | FPYYALRFIDDLELLKDIRFKIKRGLGKEFFHTHETATQPVVRNKKVFTFRR |
| | | FLEVYEGERKEPDNNLWHPAPAYAFEKDGNIKVKITKNEETSKSKDDTSSD |
| | | DIAYAELSVYELRNLVYCCLNGKKDAANNIIRDYVFNYKAFLKDLENKDFS |
| | | EIDDYTAQLEERKQQLQNKLSEYNLQLHQLPKKIRKILLDEKIQDYKSHTIQ |
| | | KIKDRQEENKRILGKIKAQKQMSKENDKDSQQKNTLKTGQLASELANDIQ |
| | | NYLPENYKLELFQYRDLQKQLAYYRRKEIYILLNQNYALTYHEQQDRNEN |
| | | FNDLYYKKKHPFLHHVLTRKDNDDIFSFAFNYFKSKEIWLEKVRKKVIGLN |
| | | DTDIPKYSELFYYFKPGTSVNEKGEKIYYRKYDDHYLNKLIQRHLKQDHVI |
| | | NIPRGILNQFICPEKESYEQKNNPIQKIADQYPSTQDFYKFPRFYHPTGEVLT |
| | | VEDINYKLVELSKDKDHPHNNDKKEHKKAYNQLKKYLKKEKTIRYIQSCD |
| | | RVLLEMEKYYLNNYFKKSNEEFELDLTDIELRDLFKYDETNESIHNKLDQK |
| | | MITLKFHLNGQSFLAEDKLNNFGKLHRYIYDERFISIFKYKGNKAFEGVKTE |
| | | SIYSQLEKILEAFAKEQLELFEYVQQFEKTITTNFENKVNQKRTEENARREK |
| | | NGKPLISEHYFPISILLSLTEEWGFISGKNRNFINTARNSAAHNKLDDKYIEM |
| | | LKDREYENDYFGAASKIFNDLTEKIRTA |
| Type VI | FIG. 50 | MTTIENFRKYNADKSFKNIFDFKGEIAPIAEKSSRNLELKLKNKVGVETSVH |
| Cas_7 | SEQ ID NO: | YFAIGHAFKQIDKEAVFDYIYDEETDSKKPHRFTSLKQFDEQFCKELKNIVS |
| | 14 | TIRNINSHYIHDFGQIKCDTLSLQLITFLKESI,ELAVIQTYLKSKESTKDAMTT |
| | | QDFFDAPDKDKKIVEFLKERFYAIDSEKKNLESYQNHINRSKYFGTLTKEQ |
| | | AIETILFGEVVDPNFKWKLNETHIAFPISVGKYLSYHACLFMLSMFLYKHEA |
| | | EQLISKIKGFKKSKNDEDKLKRNIFTFFSKKFSSEDIKSEQAHLVKFRDIVQY |
| | | LNHYPLDWNKYIELESAYPSMTDKLKAKIIEMEEDRSYPNFVGNTRFHTYEK |
| | | FELWGKKFFGNKIFKEYCDCSFTPKELEEFKYEKDTCGKVKDAELKLKEKH |
| | | LLKHDEIKKLEDKIEENKDKPNNITLTLDTRIKKNLLFTSYGRNQDRFMQFA |
| | | TRYLAETNYFGKDAQFKMYRFFSSVDNTNEIESQKEKLDKKLINKKQFDNL |
| | | RFHDGRLTYFATFKEHLVRYENWDTPFVEENNAVQVQITFNYEEILKDTNQ |
| | | TILVYITKVISIQRSLMVYFLEDALKSNTLANSEGVGVKLLFNYYMHHKKEF |
| | | AENKHELENNDKESIDNTYKKIFPKRLINKFVAVSPNDPKQQSVYESILEKA |
| | | KKSEERYKDLRAKAEKDKRLEDFDKRNKGKQFKLQFVRKAWHLMYFRDI |
| | | YNLYAIDGKPENHHKHLHITREEFNNFCRYMFAFDEVPQYKLLLKNMLAE |
| | | KHFLDNKAFETLFDSSHDLNSMYCKTKEKFKVWMSQPKETSNDKEHYTLA |
| | | NYEKFFKDKMFYINLSHFRDFLKEKKRFIIANDKIVFKSLENNQYLMQDYYI |
| | | EETPAKEKYKTKEEYKANKNLYNELRKSRLEDALLYEMAMHYLGMEKDI |
| | | TKNAKVPVQKILSQDVSFEEKDLKNITNYTLSVPFKKLESYLGLMAFKEKQE |
| | | QEYKGSYMINLVEYLKKIEQDKDTKKEIKQIWNDINGNKKLSLDQLNKFDA |
| | | HIISNSIKFTRVAILFEQYFIVKHNHSIIKDNRISFEEIEEIKEYFVKLTRNKAFH |
| | | FNIPEKPYSSLLKEIEKRFIQKEVKIQNPKSFDEIKLNEKYICSAFLNSLYDVY |
| | | FNFKEKDEKKKRYDAEQKYFTAIIA |
| Type VI | FIG. 53 | METTQTSENKRRSLATDPQYFGGYLNMARLNIYNINNYLAEEFGLSQLPED |
| Cas_8 | SEQ ID NO: | GYEKNSFLCNQKQTKLNWNRVFSKAVTFLPILKVFDSESLPKSEKEDKSTPE |
| | 15 | TGKDFAKMADSLKVLFSEIQEFRNDYSHYYSTEKGTDRKITISNELADFLKF |
| | | NYKRAIEYTRVRFKDVYTDDDFNVAANKKMVIGGVITTEGLVFLTSMFLE |
| | | REYAFQFIGKITGLKGTQYVGFRAFRDVLMAFCIKLPHEKLKSDDFIQSFTL |
| | | DIINELNRCPKTLYNVITEEEKRKFRPQIEPEKIDNLLKNSGIELEEYDENFDD |
| | | YVESLTRKIRHENRFNYFALRYEDENKIFGKYRFQIDLGKLVIDEYPKKFFNE |
| | | EVQRRIIENAKAFDKLSDLVDETAILKKIDIQNHQVYFEPFAPHYNTENNKI |
| | | ALLSKSDIARVRKVKTKTGVERKNLFQPLPEAFLSCAELYKIVLLEYLKPGE |
| | | AEKLVTDFILANNSKLMNMQFIELVKKQMPGWIVFQKETDTKSRLAYSQIN |
| | | FNELLSRKSQLNKVLAEHNLNDKQIPSKILEFWLNISDVKQQFTTGERIKLIK |
| | | RDCMKRLKALKKFKTTGKGKIPKIGEMATFLAKDIVDMVIGKEKKQKITSF |
| | | YYDKMQECLALYADPEKKKTFIHIITHELGLYEKDGHPFLNRINFNELRYTR |
| | | DIYEKYLEEKGEKMVKFYNARRGNYTEKDKSWLRETFYTLVEKEIKGKKR |
| | | IMTEVVLPSDKSKIPFTLLQLEEKTTYSLADWLQNITKGKEHGDGKKPVNLP |
| | | TNLFDETITSLLKTELDNKQALYPENAKMNELFKLWWMGRGDGVQHFYD |
| | | AEREYFVFEQPVKFKPGSKAKFSDYYCIALTKAFKEKEKTATKERKQAPEL |
| | | DEVEKTFQQAIAGTEKEIRELQEEDRVCALMLEKLISREKHITVKLESIENLL |
| | | KESVVVKQTVNGKLYFDENGNEIKDKSNPVITKTIVDKRKGKDYGLLRKF |
| | | ANDRRVPELFEYFSGEEIPLEQLKKELDGYNIAKHLVFDVVFRLEEKLEKSN |
| | | RNEIISYFTDDKGNAKGGNIQHLPYLNLLKEKDLVTPGEMAFLNMVRNCFS |
| | | HNQFPKKSIMKKVVKPGENNFAKKIADIYNEKIEALILKLA |

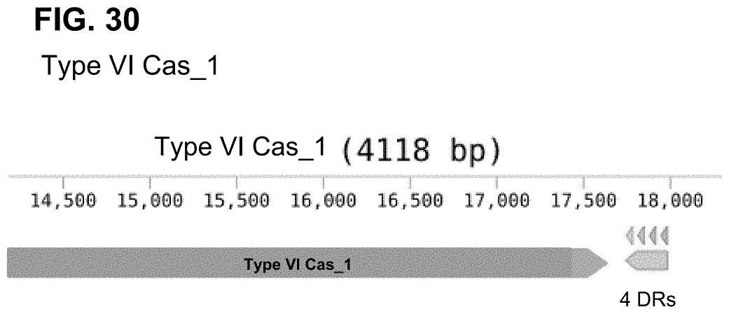

SEQ ID NO: 8 represents a novel Type VI variant of the disclosure, Type VI Cas_1, (1148 amino acids in length). FIG. 30 is a schematic representation of the organization of the CRISPR Cas cluster loci around the Type VI Cas_1 gene of the disclosure. FIG. 32 shows the amino acid sequence of Type VI Cas_1 (SEQ ID NO: 8) with the HEPN motifs underlined/highlighted. The HEPN motifs (E . . . RxxxxH (SEQ ID NO: 93)) I and II are sequentially shown (highlighted in gray).

In some embodiments the Type VI CRISPR-Cas RNA-guided endonuclease of the disclosure comprises the amino acid sequence of SEQ ID NO: 8 and proteins with at least 30%-99.5% sequence identity thereto. Accordingly, provided herein are proteins comprising the amino acid sequence of SEQ ID NO: 8 and proteins with at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% or at least 99.5% sequence identity thereto. Also provided herein are nucleic acids encoding the proteins comprising the amino acid sequence of SEQ ID NO: 8 and proteins with at least 30%-99.5% sequence identity thereto.

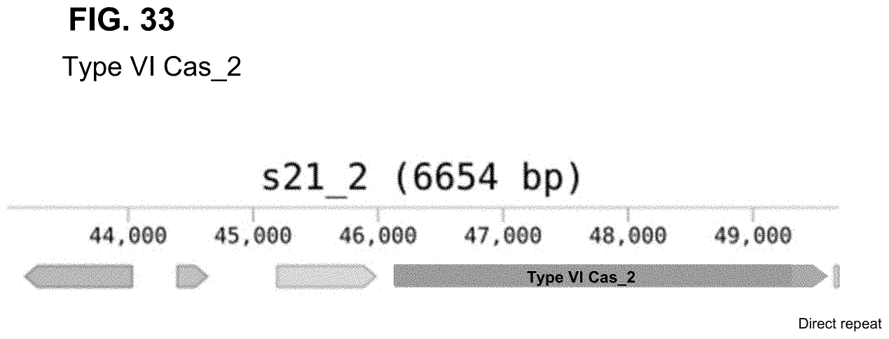

SEQ ID NO: 9 represents a novel Type VI variant of the disclosure, Type VI Cas_2, (1138 amino acids in length). FIG. 33 is a schematic representation of the organization of the CRISPR Cas cluster loci around the Type VI Cas_2 gene of the disclosure. FIG. 35 shows the amino acid sequence of Type VI Cas_2 (SEQ ID NO: 9) with the HEPN motifs underlined/highlighted. The HEPN motifs (E . . . RxxxxH (SEQ ID NO: 93)) I and II are sequentially shown (highlighted in gray).

In some embodiments the Type VI CRISPR-Cas RNA-guided endonuclease of the disclosure comprises the amino acid sequence of SEQ ID NO: 9 and proteins with at least 30%-99.5% sequence identity thereto. Accordingly, provided herein are proteins comprising the amino acid sequence of SEQ ID NO: 9 and proteins with at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% or at least 99.5% sequence identity thereto. Also provided herein are nucleic acids encoding the proteins comprising the amino acid sequence of SEQ ID NO: 9 and proteins with at least 30%-99.5% sequence identity thereto.

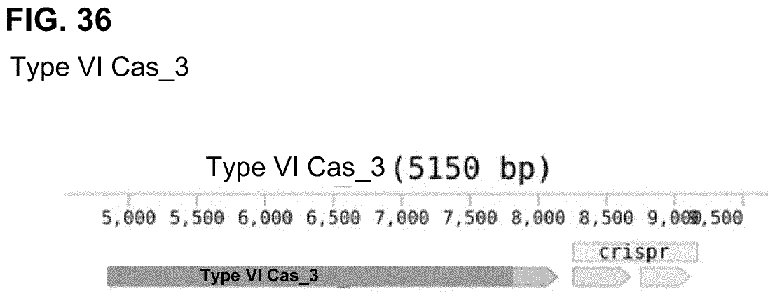

SEQ ID NO: 10 represents a novel Type VI variant of the disclosure, Type VI Cas_3, (1093 amino acids in length). FIG. 36 is a schematic representation of the organization of the CRISPR Cas cluster loci around the Type VI Cas_3 gene of the disclosure. FIG. 38 shows the amino acid sequence of Type VI Cas_3 (SEQ ID NO: 10) with the HEPN motifs underlined/highlighted. The HEPN motifs (E . . . RxxxxH (SEQ ID NO: 93)) I and II are sequentially shown (highlighted in gray).

In some embodiments the Type VI CRISPR-Cas RNA-guided endonuclease of the disclosure comprises the amino acid sequence of SEQ ID NO: 10 and proteins with at least 30%-99.5% sequence identity thereto. Accordingly, provided herein are proteins comprising the amino acid sequence of SEQ ID NO: 10 and proteins with at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% or at least 99.5% sequence identity thereto. Also provided herein are nucleic acids encoding the proteins comprising the amino acid sequence of SEQ ID NO: 10 and proteins with at least 30%-99.5% sequence identity thereto.

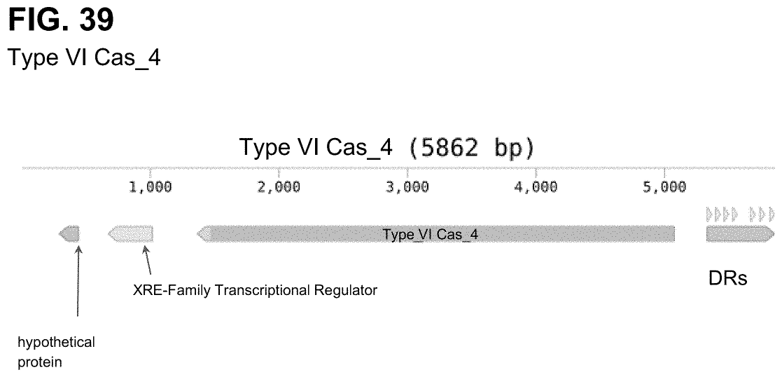

SEQ ID NO: 11 represents a novel Type VI variant of the disclosure, Type VI Cas_4, (1236 amino acids in length). FIG. 39 is a schematic representation of the organization of the CRISPR Cas cluster loci around the Type VI Cas_4 gene of the disclosure. FIG. 41 shows the amino acid sequence of Type VI Cas_4 (SEQ ID NO: 11) with the HEPN motifs underlined/highlighted. The HEPN motifs (E . . . RxxxxH (SEQ ID NO: 93)) I and II are sequentially shown (highlighted in gray).

In some embodiments the Type VI CRISPR-Cas RNA-guided endonuclease of the disclosure comprises the amino acid sequence of SEQ ID NO: 11 and proteins with at least 30%-99.5% sequence identity thereto. Accordingly, provided herein are proteins comprising the amino acid sequence of SEQ ID NO: 11 and proteins with at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% or at least 99.5% sequence identity thereto. Also provided herein are nucleic acids encoding the proteins comprising the amino acid sequence of SEQ ID NO: 11 and proteins with at least 30%-99.5% sequence identity thereto.

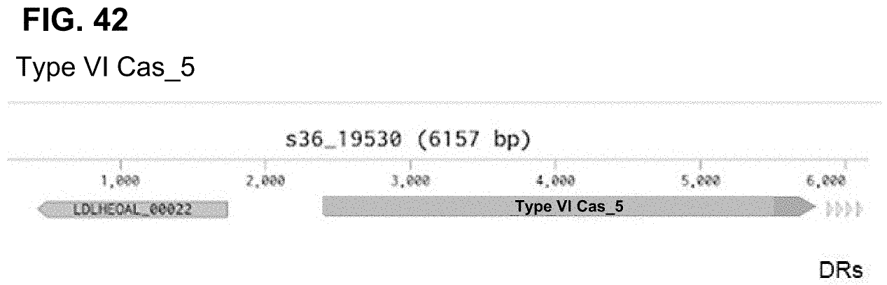

SEQ ID NO: 12 represents a novel Type VI variant of the disclosure, Type VI Cas_5, (1092 amino acids in length). FIG. 42 is a schematic representation of the organization of the CRISPR Cas cluster loci around the Type VI Cas_5 gene of the disclosure. FIG. 44 shows the amino acid sequence of Type VI Cas_5 (SEQ ID NO: 12) with the HEPN motifs underlined/highlighted. The (E . . . CNxxxH (SEQ ID NO: 142)) motif was previously observed aligned with HEPN motif (Anantharaman et al. Biology Direct 2013, 8:15). The HEPN (E . . . RxxxxH (SEQ ID NO: 93)) and (E . . . CNxxxH (SEQ ID NO: 142)) motifs are shown in gray.

In some embodiments the Type VI CRISPR-Cas RNA-guided endonuclease of the disclosure comprises the amino acid sequence of SEQ ID NO: 12 and proteins with at least 30%-99.5% sequence identity thereto. Accordingly, provided herein are proteins comprising the amino acid sequence of SEQ ID NO: 12 and proteins with at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% or at least 99.5% sequence identity thereto. Also provided herein are nucleic acids encoding the proteins comprising the amino acid sequence of SEQ ID NO: 12 and proteins with at least 30%-99.5% sequence identity thereto.

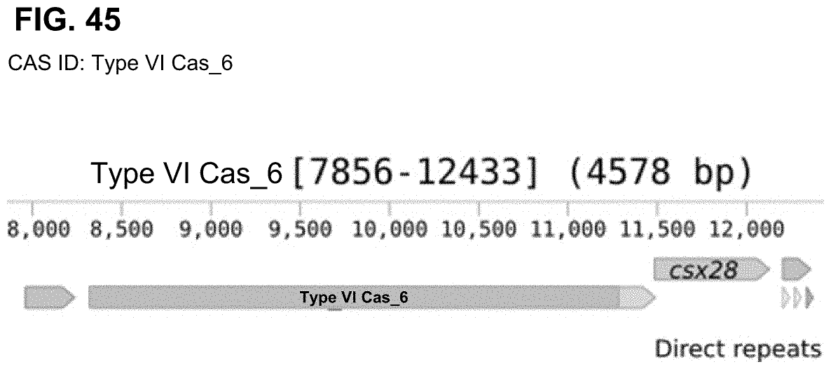

SEQ ID NO: 13 represents a novel Type VI variant of the disclosure, Type VI Cas_6, (1053 amino acids in length). FIG. 45 is a schematic representation of the organization of the CRISPR Cas cluster loci around the Type VI Cas_6 gene of the disclosure. FIG. 47 shows the amino acid sequence of Type VI Cas_6 (SEQ ID NO: 13). The HEPN motifs (E . . . RxxxxH (SEQ ID NO: 93)) I and II are sequentially shown (highlighted in gray).

In some embodiments the Type VI CRISPR-Cas RNA-guided endonuclease of the disclosure comprises the amino acid sequence of SEQ ID NO: 13 and proteins with at least 30%-99.5% sequence identity thereto. Accordingly, provided herein are proteins comprising the amino acid sequence of SEQ ID NO: 13 and proteins with at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% or at least 99.5% sequence identity thereto. Also provided herein are nucleic acids encoding the proteins comprising the amino acid sequence of SEQ ID NO: 13 and proteins with at least 30%-99.5% sequence identity thereto.

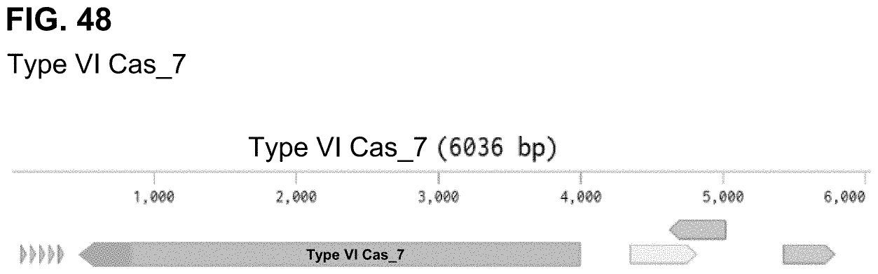

SEQ ID NO: 14 represents a novel Type VI variant of the disclosure, Type VI Cas_7, (1163 amino acids in length). FIG. 48 is a schematic representation of the organization of the CRISPR Cas cluster loci around the Type VI Cas_7 gene of the disclosure. FIG. 50 shows the amino acid sequence of Type VI Cas_7 (SEQ ID NO: 14). The HEPN motifs (E . . . RxxxxH (SEQ ID NO: 93)) I and II are sequentially shown (highlighted in gray).

In some embodiments the Type VI CRISPR-Cas RNA-guided endonuclease of the disclosure comprises the amino acid sequence of SEQ ID NO: 14 and proteins with at least 30%-99.5% sequence identity thereto. Accordingly, provided herein are proteins comprising the amino acid sequence of SEQ ID NO: 14 and proteins with at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% or at least 99.5% sequence identity thereto. Also provided herein are nucleic acids encoding the proteins comprising the amino acid sequence of SEQ ID NO: 14 and proteins with at least 30%-99.5% sequence identity thereto.

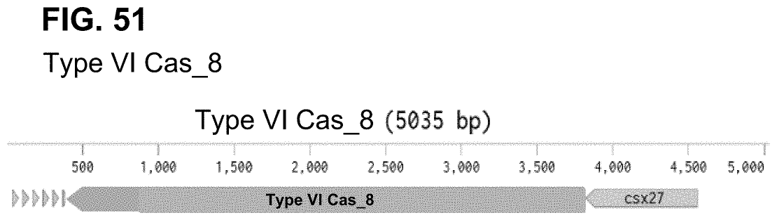

SEQ ID NO: 15 represents a novel Type VI variant of the disclosure, Type VI Cas_8, (1124 amino acids in length). FIG. 51 is a schematic representation of the organization of the CRISPR Cas cluster loci around the Type VI Cas_8 gene of the disclosure. FIG. 53 shows the amino acid sequence of Type VI Cas_8 (SEQ ID NO: 15). The HEPN motifs (E . . . RxxxxH (SEQ ID NO: 93)) I and II are sequentially shown (highlighted in gray).

In some embodiments the Type VI CRISPR-Cas RNA-guided endonuclease of the disclosure comprises the amino acid sequence of SEQ ID NO: 15 and proteins with at least 30%-99.5% sequence identity thereto. Accordingly, provided herein are proteins comprising the amino acid sequence of SEQ ID NO: 15 and proteins with at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto. Also provided herein are nucleic acids encoding the proteins comprising the amino acid sequence of SEQ ID NO: 15 and proteins with at least 30%-99.5% sequence identity thereto.

Table 6 provides exemplary nucleic acid sequences for encoding certain Type VI sequences of the disclosure. Also provided are exemplary E. coli codon optimized nucleic acid sequences for encoding certain Type VI sequences of the disclosure.

Accordingly, provided herein are exemplary nucleic acid sequences encoding the Type VI CRISPR-Cas RNA-guided endonucleases of the disclosure. In some embodiments, a Type VI CRISPR-Cas RNA-guided endonuclease is encoded by a nucleic acid sequence comprising or consisting of the sequence of any one of SEQ ID NOs: 35-50, or a nucleic acid sequence with at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

TABLE 6

| NAME | | |
|---|---|---|
| SEQUENCE | NUCLEIC ACID SEQUENCE | CODON OPTIMIZED NUCLEIC ACID |
| Type VI | ATGACAGAAAATATATCCACTGAAAAACAAAC | ATGACCGAGAACATCAGCACCGAAAAACAGACCGCGT |
| Cas_1 | TGCATATAAAATACAGAACTCAAGTGACAAGC | ACAAGATTCAAAACAGCAGCGACAAGCACTTCTTTGCG |
| | ACTTCTTTGCATCCTTTCTAAATCTTGCAGTGAA | AGCTTCCTGAACCTGGCGGTTAACAACGTGGAGAACGC |
| | TAATGTAGAAAATGCTTTTGATGAATTTGCAAA | GTTCGATGAATTTGCGAAGCGTCTGGGTGTTAGCAACA |
| | ACGATTAGGAGTTTCAAATTCTAATAAAAAAGG | GCAACAAGAAAGGCGAGCGTTACAAACCGGACGAAAG |
| | CGAGAGATATAAACCTGATGAAAGCATTAAAC | CATTAAACAGTTCTTTAAGCCGGAGCTGAGCCTGACCG |
| | AGTTTTTCAAACCTGAGTTATCATTAACTGATTG | ATTGGGAAAAGCGTGTGGACATGCTGGAGCAATACTTC |
| | GGAAAAACGTGTGGATATGCTTGAACAATATTT | CCGCTGGTTAGCTATCTGAAGGGTAACGTGACCGATAA |
| | TCCGCTTGTAAGTTACCTTAAGGGAAATGTAAC | CAACGAAAAGGACAGCAAAAGCAAGATCCTGAAATGC |
| | AGATAATAATGAAAAGGATAGCAAATCTAAAA | GATTTTAGCAGCCACGACGAGATGAAGAAAGCGTTCGC |
| | TACTTAAATGTGATTTTTCATCACATGATGAAA | GAACTACCTGACCTATCTGGTTAAAGCGCTGGACGATC |
| | TGAAGAAAGCATTTGCTAATTATCTCACATATT | TGCGTAACTACTATACCCACTTTTACCACGATCCGATTA |
| | TAGTAAAAGCTTTAGATGATTTGAGAAATTATT | AATTCAAGCCGGAGGACAAGAAATTCTATGAATTTCTG |
| | ATACCCATTTTTATCATGATCCCATAAAATTTAA | GATGAGCTGTTTGTGGAAGTTATCAAGGATGTGCGTAA |
| | ACCTGAAGATAAAAAGTTTTATGAGTTCCTGGA | GAAAAAGAAAAAGAGCGACAAAACCAAGGAAGCGCTG |
| | TGAGCTTTTTGTAGAGGTAATAAAAGATGTAAG | AAAGATGAGCTGGAAATCGAGTTCGAGGAACGTATGA |
| | AAAAAAGAAGAAGAAATCTGATAAAACTAAAG | AAGACAAGAGCGCGGCGCTGGAGAAGATGGACAAAGA |
| | AAGCCCTTAAAGATGAACTTGAAATTGAGTTTG | TGCGGGCAAAAAGGTTAAGAACCGTAGCGAAGACGAG |
| | AGGAGCGCATGAAAGACAAAAGTGCTGCTCTC | CTGCGTAACGCGGTGATGAACGATGCGTTTAAACACCT |
| | GAAAAAATGGATAAAGATGCAGGTAAAAAGGT | GATCGCGAAAGACAAGGATGAGTACAGCCTGATTGAA |
| | CAAAAATAGAAGCGAAGATGAGCTGAGAAATG | CGTTATCAGGCGTTCCCGGAAAACCTGGACGCGCCGAT |
| | CTGTAATGAATGATGCTTTCAAGCATCTGATTG | TAGCGAGAAGAGCCTGATGTTTCTGTGCAGCTGCTTCC |
| | CAAAGGATAAGGATGAATATTCTCTAATAGAA | TGAGCCGTCGTGATATGGAGCTGTTTAAGGCGCGTATC |
| | AGGTATCAGGCATTTCCTGAGAATCTGGATGCT | ACCGGTTTCAAAGGCAAGATGGTTGAAGGCGAGGACA |
| | CCTATTTCAGAAAAGTCTCTCATGTTTTTGTGCT | GCCTGAAATACATGGCGACCCACTGGGTGTACAACTAT |
| | CATGCTTTTTATCCAGACGGGATATGGAGCTGT | CTGAACTTCAAGGGCCTGAAGCGTAAGATCAACACCCG |
| | TTAAAGCTCGAATTACAGGTTTTAAAGGCAAAA | TTTTGAAAAAGAGAACCTGCTGTTCCAGATTGTTGATG |
| | TGGTTGAAGGAGAAGATAGTTTAAAATACATG | AACTGAGCAAAGTGCCGGACTGCCTGTACCGTGTTATC |
| | GCCACACATTGGGTATATAATTACCTGAATTTT | AAAGATAAGAACGAGTTTCTGCTGGACATTAACAAGTT |
| | AAAGGGCTTAAACGAAAAATCAACACCCGTTTT | CTATAAACAAACCAAGGGTGAAGCGGAGAGCCCGGAA |
| | GAGAAAGAAAACCTCCTGTTTCAAATTGTTGAT | AACGAGGAAGTGGTTAACCCGATCATTCGTAAACGTTT |
| | GAACTGAGCAAAGTACCGGACTGCCTTTATCGG | TGAGGACAAGTTCAACTACTTTGCGCTGCGTTATCTGG |
| | GTTATTAAGGATAAAAACGAATTCTTACTCGAT | ATGAGTTCGCGGGTTTTGAAAACCTGAAGTTCCAGATC |
| | ATAAACAAGTTTTATAAACAAACAAAAGGCGA | TACGCGGGCAACTATCTGCACCACAAACAAGAAAAGA |
| | GGCTGAAAGTCCGGAAAACGAAGAGGTGGTTA | CCAGCGCGCAGACCCAACTGAAGACCGACCGTAAAAT |
| | ATCCAATAATAAGAAAACGGTTTGAGGATAAA | CAAGGAGAAAATTAACGTTTTCGGTAAACTGAGCGATG |
| | TTCAACTACTTTGCCTTACGCTACCTTGATGAAT | TGAACAAGGCGAAAGCGAACTTCTTTGCGAACAAAACC |
| | TTGCCGGTTTTGAAAACCTGAAATTTCAGATAT | GAGGACAGCGATATGGACGAAGGCCTGGAGGAATACC |
| | ACGCCGGAAACTACCTCCATCACAAGCAAGAA | CGAACCCGAGCTATAACATCAACGGTGGCAGCATCCTG |
| | AAGACAAGTGCCCAAACGCAACTTAAAACAGA | ATTCACCTGAACCTGAACAAGTACCGTTATGGTCAGGA |
| | TAGAAAAATCAAGGAAAAAATTAATGTTTTTGG | GTTCCACGAGCTGAAACAACTGCGTATCGAAAAGGAG |
| | GAAATTATCTGATGTCAACAAAGCAAAGGCAA | AAACGTGGCGAAAACAAAACCGACAAGATTAGCATCA |
| | ACTTTTTTGCAAACAAAACCGAGGATAGCGACA | TTAAGGACCTGTTCGAGGACAACACCGAAATCAAAGA |
| | TGGATGAGGGCTTGGAAGAATATCCAAATCCCT | GGAAGATTGGGTTTTCCCGGTGGCGCTGCTGAGCCTGA |
| | CATACAACATTAATGGAGGGAGCATTTTGATAC | ACGAACTGCCGGCGCTGCTGTACGAGATGCTGGTTAAC |
| | ACTTAAATTTGAACAAATATAGATATGGGCAAG | AAAAAGAGCAGCAAGGACATCGAGCAGATCATTGCGG |
| | AATTCCATGAATTGAAACAGTTGCGTATTGAAA | ACCGTATCGTGAGCCACTACAAAAAGATTAAGGATTTC |
| | AGGAAAAACGTGGGGAGAATAAAACAGATAAA | GAGGGCACCGCGGATGAACTGAAGGACAAAAACCTGC |
| | ATTTCAATTATTAAAGATTTGTTTGAAGATAAT | CGGTTAACCTGCGTAAGGCGTTCGGCGCGGACGATAAA |
| | ACTGAAATCAAAGAAGAAGATTGGGTCTTCCCT | AACACCGACAAGCTGGAAAACGCGATCACCAAAGATA |
| | GTTGCCTTATTGTCTCTAAATGAACTGCCCGCTT | TTGAAGCGGGCGAGGACAAACTGCAGCTGATTAAGGA |
| | TGTTGTATGAAATGCTCGTAAATAAGAAAAGTT | GAACACCCGTGAAATGCGTAGCAACAACCGTAAGTAC |
| | CGAAGGATATTGAACAAATCATTGCAGACAGG | GTGTTTTATCTGAAGGAGAAAGGCGAGGAAGCGACCTG |
| | ATTGTTTCGCATTACAAGAAAATAAAAGATTTT | GCTGGCGAAAGACATCAAGCGTTTCATGCCGGAAAACG |
| | GAAGGTACTGCAGATGAGTTAAAAGACAAAAA | CGAAAAACCAGTGGAAGAGCTACAACCACAACGAGCT |
| | TCTGCCTGTTAATTTACGTAAAGCTTTTGGTGCT | GCAAAAGGGTCTGGCGTACTATGAACTGGAGCGTCAGA |
| | GATGATAAAAATACTGATAAACTGGAAAATGC | ACGTTCTGGCGCTGCTGGAAAGCAAATGGGATATGGAC |
| | CATTACCAAGGACATAGAAGCAGGAGAAGATA | AGCTGCCACCCGCACTGGGGTGAGGACCTGAAGGAACT |
| | AGCTTCAGCTGATCAAAGAGAATACAAGAGAA | GTTTATTACCCACAGCCGTTTCGACGATTTTTACAAAGC |
| | ATGCGCAGTAATAACCGCAAATATGTATTTTAT | GTATATGCTGTGCCGTCAGGGCTTCCTGGAGCAATTTA |
| | TTAAAAGAGAAAGGGGAAGAAGCAACATGGCT | AGACCCTGGTTATCCGTAACAAAAGCGACAAAAAGCTG |
| | GGCAAAGGACATTAAGCGATTTATGCCTGAAA | CTGAACAAAGTTCTGAAGGATGTGTTCATCCCGTACAA |
| | ATGCAAAAAATCAATGGAAGTCGTATAATCAC | AAAGCGTTTCTTTGTGATTAACAGCCTGGAAAACGAGA |
| | AATGAATTGCAAAAGGGGCTGGCTTATTATGAA | AAAAGGCGCTGCTGAGCCACCCGATTGTTCTGCCGCGT |
| | CTTGAAAGACAAAATGTTTTGGCTCTGCTTGAA | GGTCTGTTTGACAACAAACCGACCTTCATCAAGGGCGT |
| | TCAAAATGGGATATGGATTCCTGTCACCCACAC | GAGCCTGGAAAACGATCCGAGCCGTTTCGCGAACTGGT |
| | TGGGGTGAAGACCTGAAAGAACTTTTTATTACG | TTGCGTACCTGCGTCAGGAAGCGAAGAACGATCACCAA |
| | CACAGCCGTTTTGATGATTTTTATAAAGCTTATA | GTTTTCTACGATTTTGAACGTGACTATGTGAAAGCGTTC |
| | TGCTTTGTCGTCAAGGATTTTTGGAGCAATTTA | AGCGAGCTGAAGGACAAAAGCAAGTACAACAACAACA |
| | AAACCCTGGTTATTAGGAATAAATCGGACAAA | AGCACTTCAACTTCAAGGTGGACAGCGAAATTCGTATG |
| | AAGCTTCTGAATAAAGTTCTTAAAGATGTTTTT | TGCCTGCAGAACGATCTGGTTCTGAAGCTGATCGTGAA |
| | ATTCCTTATAAAAAACGATTTTTTGTAATCAAT | AAAGCTGTTCAAAGGTATCTTTGATGTTGACGAAAACA |
| | AGCCTTGAAAATGAAAAGAAGGCATTGTTAAG | TTAAGCTGAACGACTTCTACCTGGAAAAAACCGAGGTG |
| | TCATCCCATTGTGTTGCCAAGAGGCTTGTTTGAT | GCGAAGCAGCGTGAGCAAGCGCTGGATCAAAACAAAC |
| | AATAAACCAACTTTCATTAAAGGGGTTTCGCTT | GTCTGAAGGGTGACGATGGCGATGTTATCTATAAAGAG |
| | GAAAATGATCCGTCACGCTTTGCAAACTGGTTT | GACCACCTGTTCCGTAAAACCTTTGCGAAGGATTTCCT |
| | GCATATTTACGACAGGAAGCCAAAAACGATCA | GAACGGCAAGCTGCACTTCGATAAATTTAAGCTGAAAG |
| | TCAGGTATTCTATGATTTTGAAAGAGACTATGT | ACTTTGGCAAAGCGCTGGTGTTCGCGGCGGACGAAAAG |
| | TAAAGCTTTTTCCGAGCTGAAAGATAAAAGTAA | GTTAAAACCCTGGTGAGCTACAGCGAGAACGCGTGGAC |
| | GTACAACAATAATAAGCACTTCAATTTCAAGGT | CCAGGAAGAGCTGCAAAAAGAACTGCACACCAATACC |
| | AGATTCAGAAATAAGAATGTGTTTGCAAAATGA | GACAGCTATGAGCGTATTCGTCAGGATGAGTTCTTCAA |
| | TCTTGTCTTAAAGTTGATTGTGAAAAAGCTTTTT | AAAGATCCACGAGCTGGAGGAAAGCATTTGGCAGAAG |
| | AAAGGTATTTTTGATGTTGATGAAAATATAAAG | CACAAACACGAACGTGAGAAACTGCAAGACAAGAGCG |
| | TTAAATGATTTCTATCTTGAAAAGACAGAAGTT | GTAACGAAAACTTTAACAACTACGTGAAAGTTGGCGTG |
| | GCAAAACAGAGAGAGCAAGCTCTTGATCAGAA | CTGGAGAAGCTGAACGATAGCTTTAAAGACGAGTTCGA |
| | TAAGCGATTAAAAGGAGATGATGGAGATGTGA | GAACCTGTATAAGGACAAAAAGAACAAGCGTATCCAG |
| | TATATAAGGAAGACCACTTGTTTCGTAAAACAT | AAGCTGCGTCAATGCAACCACGTGGTTCAGAAAGCGTA |
| | TTGCTAAAGATTTTCTAAACGGCAAATTGCATT | CTGCCTGGTTCAACTGCGTAACAAGTTCAGCCACAACC |
| | TCGACAAATTTAAATTGAAAGATTTTGGTAAAG | AGCTGCCGCCGAAACAACTGTTCGACTTTATGACCGAA |
| | CTCTGGTATTTGCAGCAGATGAAAAAGTAAAAA | ACCCTGGCGGAAAAGGATAAACAGACCTACAGCCGTT |
| | CTTTAGTTTCTTATTCGGAAAACGCCTGGACAC | ATTTTATGGATGTTACCGACAAGATGGTGCAAGAGTTC |
| | AGGAAGAGTTACAGAAGGAATTACATACAAAT | AAACCGCTGGTGTAG (SEQ ID NO: 36) |
| | ACCGACTCTTATGAGCGCATACGGCAAGATGAG | |
| | TTTTTTAAAAAAATTCATGAGCTTGAAGAATCT | |
| | ATTTGGCAAAAGCATAAACATGAAAGAGAAAA | |
| | GTTACAAGACAAAAGTGGTAATGAAAATTTCA | |
| | ATAATTATGTAAAAGTTGGAGTGCTGGAAAAGC | |
| | TGAACGATTCATTTAAGGATGAATTTGAAAACT | |
| | TATATAAAGATAAAAAAAATAAAAGAATTCAA | |
| | AAACTCAGGCAATGTAACCATGTCGTTCAAAAA | |
| | GCATACTGCCTTGTGCAGCTTAGAAATAAGTTT | |
| | TCACACAATCAGTTGCCTCCAAAACAACTGTTT | |
| | GATTTTATGACTGAAACCCTGGCTGAAAAAGAC | |
| | AAGCAAACATACAGCCGTTATTTTATGGATGTT | |
| | ACTGATAAAATGGTGCAGGAATTTAAGCCACTG | |
| | GTTTAG (SEQ ID NO: 35) | |
| Type VI | ATGGAAACACAAATTGTAAACAAAAAAAGAAC | ATGGAAACCCAGATCGTTAACAAGAAACGTACCCTGAA |
| Cas_2 | CTTAAAAGATGACCCACAGTACTTTGGCACTTA | AGACGATCCGCAATACTTCGGCACCTATCTGAACATGG |
| | TCTAAATATGGCAAGACACAATATTTTCTTAAT | CGCGTCACAACATCTTTCTGATTGAGAACCACATTGCG |
| | TGAAAATCATATTGCACAAAAGTTTGAAAAAA | CAGAAATTTGAAAAGAACAAACTGGGCGTGGTTAAGA |
| | ATAAATTGGGAGTTGTTAAAAGCGATGAACAC | GCGACGAGCACATCGCGAGCCGTCAGTTCTTTGATGCG |
| | ATTGCAAGCCGACAGTTTTTTGATGCTGCTTTTA | GCGTTCAAAAACAACAAGCTGGCGAACAGCAAACAAA |
| | AAAATAATAAACTAGCAAATAGCAAACAGATT | TCTTCAACGCGTTTACCCGTTTCATCCACGTGGCGAAGA |
| | TTTAATGCCTTTACTAGATTTATTCATGTTGCTA | TTTTTGACAACGATCTGCTGCCGAAAAGCGAAAAGCAG |
| | AAATTTTCGATAACGATTTATTGCCTAAATCAG | GAAGAGGGTTTTCAGCAAGACAGCATTGATTTCAACCT |
| | AAAAACAAGAAGAAGGCTTTCAGCAAGATAGT | GCTGAGCGAAACCTTCTTCAGCTGCTTCAAGGAACTGA |
| | ATAGACTTCAACTTGCTATCAGAAACCTTTTTC | ACCAATTTCGTAACAACTTCAGCCACTACTATCACATC |
| | AGTTGTTTTAAAGAGTTAAATCAATTTAGAAAC | GAGAACGAGGAAAAACGTAACCTGTTTGTTAGCGAAA |
| | AACTTCTCTCACTATTACCATATAGAAAACGAA | CCCTGAAGTACTTCGTGATCAAAGCGTATGAGAAGGCG |
| | GAAAAAAGAAATCTATTTGTAAGTGAAACTTTA | ATTGCGTACGCGGAACAGCGTTTTAAAGACGTTTTCAA |
| | AAATACTTTGTAATTAAGGCTTATGAGAAAGCA | GCACGAGCACTTCAACATCGCGCGTAACAAGAAACTGT |
| | ATTGCTTATGCTGAACAACGATTTAAGGACGTA | TTACCCTGCACCAAGAGTTCACCCGTGATGGTCTGGTG |
| | TTCAAGCACGAACATTTTAATATAGCACGTAAT | TTCTTTTGCTGCCTGTTTCTGGAAAAAGAGTACGCGTTC |
| | AAAAAGTTATTTACTCTTCACCAAGAATTTACT | CACTTTATCAACAAAATCATTGGCTTTAAGGACACCCG |
| | AGAGATGGCTTAGTGTTTTTTTGCTGTCTGTTTT | TACCGCGGAGTTCAAGGCGACCCGTGAAGTGTTTAGCG |
| | TAGAGAAAGAATATGCCTTTCATTTTATCAACA | TTTTCTGCGTGACCCTGCCGCACAACCGTTTCATCAGCG |
| | AAATAATTGGTTTTAAAGACACCCGAACCGCAG | AGGACCCGGCGCAGGCGTATATTCTGGATGCGCTGAAC |
| | AATTTAAAGCCACTCGAGAAGTGTTTTCTGTTTT | TACCTGCACCGTTGCCCGACCGAGCTGTATAACAACCT |
| | CTGTGTTACATTACCCCACAATCGCTTTATAAG | GAGCGAAGACGCGAAGAAACACTTTCAGCCGACCCTG |
| | CGAAGACCCCGCACAAGCTTATATTTTAGATGC | AGCTACGAAGCGGTTCAGAACATTCAAGGTAGCAGCGT |
| | GCTAAACTATTTGCATCGTTGCCCAACTGAACT | GAACAACGAGCAACTGCCGATCGAAGATTTTGACGATT |
| | CTACAATAACTTGAGTGAAGATGCTAAAAAGC | ACATCCAGAGCATTACCACCCAAAAACGTAACACCGAC |
| | ATTTTCAACCCACCCTTAGTTATGAGGCAGTAC | CGTTTCCCGTTCTTTGCGCTGAAGTATCTGGATAACAAA |
| | AAAATATTCAAGGCAGCAGCGTTAATAATGAA | GAGAGCTTTAAGCCGCTGTTCCACCTGCACCTGGGTAA |
| | CAACTTCCTATTGAAGATTTTGATGATTATATAC | ACTGCTGCTGAAGAGCTACAAGAAAAACCTGCTGGGCA |
| | AAAGCATTACCACACAAAAAAGAAATACCGAC | ACGAGGAAGACCGTTTTATCGTTGAGAGCTTTACCACC |
| | CGCTTCCCATTTTTTGCCTTAAAATATTTAGATA | TTCGGCACCCTGGAAAACTTCCAGCTGAGCAACATTGA |
| | ATAAAGAAAGTTTTAAACCCCTGTTTCATCTGC | GGAAGAGAACAAAGAAGAGAAAGTGCGTGAAATCACC |
| | ATTTAGGTAAGCTATTATTAAAATCTTACAAGA | CAGCTGAAGAAAGAGATCACCATTGAACAATACGCGC |
| | AAAATCTTTTAGGCAATGAAGAAGACCGCTTTA | CGAAATATCACATCGCGAACAACAAGATTGCGCTGAAC |
| | TAGTAGAAAGCTTTACCACTTTTGGTACTCTTG | CTGAGCAACAACAAATACTATAACGGTAACTTTCTGAG |
| | AAAACTTTCAATTGAGTAATATAGAAGAAGAA | CTTCCACCCGGAAGTGTTCCTGAGCATTCACGAACTGC |
| | AACAAAGAAGAAAAAGTGCGTGAAATAACTCA | CGAAAGTTGCGCTGCTGGAGCACCTGCTGCCGGGCAAG |
| | ACTTAAAAAAGAGATTACAATAGAACAATACG | GCGACCCAGCTGATCGAAAACTTTGTTAACCTGAACAG |
| | CCCCTAAATACCATATAGCTAACAATAAAATTG | CAGCCACATCCTGAACAGCCAATTCATTGAAGAGGTGA |
| | CTTTAAACCTAAGCAATAATAAATACTACAACG | AGAGCAAACTGACCTTTACCCGTCCGCTGAAGAAACAG |
| | GAAATTTTCTCAGTTTTCATCCCGAAGTTTTTCT | TTCCACAAGGACAAACTGACCATTTACAACTATACCCT |
| | TAGCATACACGAATTACCTAAAGTAGCACTCTT | GCAGCAACTGAACAACAAAATCAACGAGATCATTCAGT |
| | AGAACATTTATTGCCCGGTAAAGCCACTCAGCT | TCATTGACGATAACAAGGAGCACGCGGACGATGAAAC |
| | TATTGAAAACTTTGTCAACTTAAATAGCAGCCA | CAAGAACCAAATCAAGAACAAGAAAAGCGAACTGAAA |
| | TATTTTAAACAGCCAATTTATTGAAGAAGTTAA | AACCTGTACTATAACCGTTACGTGGTTCAGGTGGTTGA |
| | ATCAAAACTCACTTTTACACGTCCACTAAAAAA | CCGTAAGCAGCAACTGGATGCGATCCTGAAAACCTATA |
| | ACAATTTCATAAAGATAAGCTTACTATTTACAA | ACCTGAACCACAAGCAGATTCCGGAGCGTATCATTAAC |
| | CTATACACTTCAACAACTGAATAATAAAATAAA | TACTGGCTGCAAATCAAAGAAGTTAAGGACGATACCAC |
| | TGAAATAATACAGTTTATTGATGACAATAAAGA | CCTGAAGAACAAAATTAAGGCGGAGAAAGAAGAGTGC |
| | ACACGCTGATGATGAAACAAAAAACCAAATAA | AAGCAGCGTCTGAAAGACCTGGCGAACCTGAAAGGTC |
| | AAAATAAAAAATCTGAGTTAAAAAATTTGTATT | CGAAGATCGGCGAAATGGCGACCTTTCTGGCGAAAGAC |
| | ACAATAGGTATGTAGTTCAAGTTGTAGATAGAA | ATCATTCACCTGGTTATCGATCTGCAGGTGAAGAAAAA |
| | AACAACAATTAGATGCTATATTAAAAACCTATA | GATTACCACCTTCTACTATGACCGTCTGCAAGAGTGCCT |
| | ACCTCAACCACAAACAAATACCCGAGCGCATC | GGCGCTGTACGCGGACATCGAAAAACAGCAAACCTTTA |
| | ATTAACTATTGGCTGCAAATTAAAGAGGTAAAA | AGCGTATTTGCAGCGAGCTGGGTCTGCTGGATGCGCTG |
| | GATGATACTACTTTAAAAAACAAAATAAAAGC | AAGGGCCACCCGTTTCTGAACCAGATCATTCTGGGTAA |
| | CGAAAAAGAAGAATGCAAACAACGGCTTAAAG | CTATAGCAAAACCAAGGACTTCTACCGTGCGTATCTGC |
| | ACTTAGCTAATCTTAAAGGCCCAAAAATTGGCG | AGCAAAAAGGCACCAACACCATCGAGAAGTACGATTA |
| | AAATGGCTACTTTCTTAGCTAAAGATATTATTC | CAACCGTAAAAAGATTGTTGAAAGCAACTGGATGTACA |
| | ATCTAGTAATAGACTTACAAGTAAAAAAGAAG | CCACCTTCTATAACGTGGAAAACAAACAGACCATCATT |
| | ATTACCACTTTTTATTACGACCGCTTGCAAGAA | AGCATCCCGAACAACAAACCGGTGCCGTACAGCTATAA |
| | TGCCTTGCCTTATATGCAGATATTGAAAAACAA | GCAGTGGCAAGCGCCGCAAACCGATTTCAACAAGTGGC |
| | CAAACCTTTAAAAGAATATGTAGCGAATTAGGT | TGAGCAACACCAGCAAGGGTATCGATAAACAGCAACC |
| | TTGTTAGATGCCTTAAAAGGACATCCGTTTTTA | GAAGCCGATTGACCTGCCGACCAACCTGTTTGATGAAA |
| | AACCAAATTATTTTAGGTAATTATTCTAAAACC | CCCTGAACAGCGCGCTGCAGCAAAAACTGCAGAACCC |
| | AAAGATTTTTATAGAGCCTACTTACAACAAAAA | GCTGCCGAACGAAAAAGCGAACTATACCGCGCTGCTGA |
| | GGCACCAATACCATTGAAAAATATGATTATAAT | AGGCGTGGATGCCGCAGAGCCAACCGTTCTACAACATG |
| | AGAAAGAAAATCGTAGAAAGCAATTGGATGTA | CCGCGTAGCTACATGGTTTATGACAACGAGGTGAACTT |
| | CACCACATTCTACAATGTGGAAAATAAACAAAC | TACCCCGGGCACCCAGGCGACCTACAAGGGT |
| | TATTATTTCCATACCCAATAATAAACCAGTGCC | TATTTCGAGAAAACCATTCAAAAGGTTCTGCGTCAGAA |
| | TTATTCTTACAAACAATGGCAAGCACCCCAAAC | AAACGAACAAATCAAAAAGGATAACCTGAAGGCGATT |
| | CGATTTTAATAAATGGCTAAGCAATACTTCAAA | AAAAAGAAACCGTTCTACACCGCGAGCCAGATCCTGGC |
| | AGGCATAGATAAGCAACAGCCAAAACCCATAG | GGTTTGCAACAACGCGATCACCGAAAACGAGAAACTG |
| | ACTTGCCCACCAATTTATTTGATGAAACACTTA | ATCCGTTTCTACGAAACCAAGGACCGTATCCTGCTGCT |
| | ATTCAGCCCTTCAGCAAAAATTACAAAACCCAT | GATTGTGCAGGAACTGAGCGGTATGCAGATGTGCCTGC |
| | TACCCAACGAAAAAGCCAATTATACAGCCTTAC | AAAAAATGGACATCAAGAGCCAGCAAAGCCCGCTGAA |
| | TGAAAGCATGGATGCCCCAAAGCCAGCCATTTT | CGAAATCATTGAGATCAAAGAAGTGATTCACCAGAAG |
| | ACAATATGCCACGCTCTTATATGGTATATGATA | ACCATTACCGCGCAACGTAAGCGTAAGGACTATACCAT |
| | ATGAGGTAAATTTTACGCCCGGTACACAAGCCA | CCTGAAGAAACTGGAGAAAGATAAGCGTCTGCCGAAC |
| | CTTATAAAGGCTATTTTGAAAAAACTATACAAA | CTGCTGCAGTACTTTGACGAAGATACCATCCCGTTCGA |
| | AAGTATTGAGGCAAAAAAACGAACAAATAAAA | CACCATTAACAAAGAGCTGTTCCACTATAACCAAAGCC |
| | AAAGACAATCTAAAAGCAATAAAGAAAAAACC | GTGAAAAGATTTTTGATAGCAGCTTCCTGCTGGAGAAA |
| | CTTTTACACGGCAAGCCAAATATTAGCGGTATG | ACCATCGTTGAAAAGCTGCAGCAAAACCAGAGCATGC |
| | TAACAATGCTATTACAGAAAATGAAAAACTAAT | ACATCCTGACCACCATGCAAGAAGAGAAAAACAAGAA |
| | TAGATTTTACGAAACCAAAGACCGCATATTGTT | AGAGGGCACCGACGTGAAGAACATCCAGTTCGATATTT |
| | GCTCATTGTTCAAGAATTAAGCGGCATGCAAAT | ACACCCAGTGGCTGCAAGAGAACAAATTTATCAGCCAA |
| | GTGCTTGCAAAAAATGGATATAAAATCGCAAC | ACCGAAGCGGACTTCCTGCTGACCGTTCGTAACAAGTT |
| | AAAGCCCCCTAAACGAAATCATAGAAATAAAA | TAGCCACAACCAGTTCCCGGAAAAAATCAAGATTGAAA |
| | GAAGTAATACACCAAAAAACCATTACTGCACA | AAGAGGTGACCTTTGATGAGAACCAGAACAAGGCGAG |
| | ACGCAAAAGAAAAGATTATACCATACTTAAAA | CCAAATCTGCGAAAACTACCACAAGAAAATTCAGGCG |
| | AGTTAGAAAAAGATAAAAGGCTGCCCAATTTA | ATCATTGCGCAACTGAACTAG (SEQ ID NO: 38) |
| | CTGCAATACTTTGATGAAGATACTATTCCATTC | |
| | GACACTATCAATAAAGAACTATTTCATTATAAC | |
| | CAAAGCCGTGAAAAGATTTTTGATAGCAGTTTT | |
| | CTTTTGGAAAAAACTATAGTAGAAAAGCTACAG | |
| | CAAAATCAAAGCATGCACATACTCACTACCATG | |
| | CAAGAAGAAAAAAATAAAAAAGAAGGCACAG | |
| | ACGTAAAAAATATTCAATTCGATATTTACACCC | |
| | AATGGCTGCAAGAAAATAAGTTCATTAGCCAA | |
| | ACCGAAGCCGATTTTTTACTTACTGTGCGCAAT | |
| | AAATTTTCACACAACCAATTTCCCGAAAAAATA | |
| | AAAATAGAAAAAGAAGTTACATTTGATGAAAA | |
| | CCAAAATAAAGCAAGCCAAATATGTGAAAACT | |
| | ACCATAAAAAAATACAAGCAATCATTGCCCAA | |
| | CTAAACTAG (SEQ ID NO: 37) | |
| Type VI | ATGGTAAATGTAAACAAAAGAACACTCACCGG | ATGGTGAACGTTAACAAACGTACCCTGACCGGTGACCC |
| Cas_3 | TGATCCGCAGTATTTTGGCGGATACCTGAATTT | GCAGTACTTTGGTGGCTATCTGAACCTGGCGCGTCTGA |
| | GGCAAGGCTAAATGTATTTGCGATTAGCAATCA | ACGTGTTCGCGATCAGCAACCACATTGCGGAGAAGATC |
| | TATTGCCGAAAAGATAAATCCATTTTTGAAGAA | AACCCGTTCCTGAAGAAAGGTAAAGTGGGCGTTCTGCA |
| | GGGAAAGGTTGGAGTATTACAGGATGACGAAA | GGACGATGAAAACATTCCGGATAGCTTTATTTGCAACA |
| | ATATTCCCGATAGTTTTATTTGCAATAAAATTA | AAATCAAGGAGAAACCGAACCTGTTCTACACCCAACTG |
| | AGGAGAAGCCGAATCTCTTTTATACACAGCTTG | GTGCGTTTCTTTCCGATCGCGCGTGTTTATGACAGCGAT |
| | TAAGGTTTTTTCCGATTGCGCGAGTTTATGATTC | CGTCTGCCGAAAGAGGAAAAGCTGCTGACCAAATGCG |
| | GGATAGATTGCCAAAGGAAGAAAAATTATTAA | AGGGTATTGACTATAGCCTGCTGACCGGCGATATGAAG |
| | CAAAGTGCGAGGGTATAGATTATTCCCTGCTTA | ATCTGCTTTAGCGAACTGAACGACTTCCGTAACGATTA |
| | CAGGGGATATGAAAATTTGTTTTTCGGAGTTGA | CAGCCACTATTTCAGCATTAAGACCGGCACCGACCGTA |
| | ATGATTTCAGGAATGATTATTCGCATTACTTTTC | AAGTTGAAATCAGCGAGCGTCTGAGCGATTTTCTGATG |
| | TATTAAAACCGGGACGGATAGGAAAGTTGAAA | ACCAACTACCTGCGTGCGATCGAGTATACCAAAGTGCG |
| | TAAGTGAAAGACTTTCGGATTTTTTAATGACTA | TTTTAAGGACGTTTACAACGATAGCCACTTCCAGATTG |
| | ATTATCTTAGGGCTATAGAATATACAAAGGTTA | CGAGCAAGCGTATCCTGGTGGACGAAAACAACATCATT |
| | GGTTTAAAGATGTTTATAATGATTCACATTTTCA | ACCCAAGATGGTCTGGTTTTCTTTATGTGCATTTTCCTG |
| | AATTGCCTCAAAGAGAATATTAGTTGACGAAAA | GAACGTGAGAGCGCGTTCCACTTTATCAACAAGATCAT |
| | TAATATTATAACACAGGATGGATTAGTTTTCTTT | TGGCTTTAAAGACACCCGTAGCCTGGATTTCAAAGCGA |
| | ATGTGCATATTTCTTGAAAGAGAAAGTGCTTTT | TGCGTGAGGTGTTCAGCGCGTTTTGCATTACCCTGCCGC |
| | CATTTTATAAATAAAATAATTGGTTTCAAAGAT | ACGACAAGTTTATCAGCGACGATGGCAAACAGGCGTTC |
| | ACGAGGTCTTTGGATTTCAAAGCGATGAGGGAA | ATTCTGGATCTGCTGAACGAGCTGAACCGTTGCCCGAA |
| | GTTTTTTCTGCTTTTTGTATTACGCTTCCGCACG | GGAACTGTTTGAGAACATCAGCAGCGAGGAAAAGAAA |
| | ATAAGTTTATAAGTGATGATGGTAAGCAGGCTT | CAGTTCCAACCGAACGTTAGCGAAAGCGCGGCGGACAT |
| | TTATACTTGATTTGCTGAATGAACTGAATAGGT | TGAGGAAAACAGCATCCCGGCGGACCTGCCGGAGGAA |
| | GTCCGAAGGAATTGTTTGAGAATATTTCAAGCG | GATTTCGAGGAATACATCCAAAGCATCATTAGCAAGAA |
| | AAGAGAAGAAGCAATTTCAGCCGAATGTGAGC | ACGTAAAACCGACCGTTTCCCGTACTTTGCGGTGAAGT |
| | GAGAGTGCAGCGGATATTGAAGAGAACAGTAT | ATCTGGATGAAAAGACCAACATCAACTTCCACCTGAAC |
| | TCCGGCTGATTTACCTGAAGAAGATTTTGAAGA | CTGGGCAAGATCGAGCTGGTTACCCGTAAGAAAAAGTT |
| | ATATATTCAAAGTATAATAAGCAAGAAAAGAA | CCTGGGTGGCGAGGAAGACCGTGACATCATTGAGGAC |
| | AGACGGACAGGTTTCCGTATTTCGCAGTAAAGT | GCGAAAGTGTTTGGCAAGCTGGGCGAATATGCGGATGA |
| | ATCTTGATGAAAAAACGAATATTAATTTTCATT | GCGTGCGGTTAGCAAACGTCTGGGCATGGAGTTCCAGC |
| | TGAATCTGGGGAAGATAGAACTTGTTACTCGCA | TGTTTAACCCGCACTACCAAATTGAGAACAACAAAATC |
| | AGAAGAAATTTTTAGGAGGAGAAGAGGATAGA | GGTTTCAGCTTTAGCCCGATTGAATGCAGCATCAAGAA |
| | GATATTATTGAGGATGCAAAGGTGTTTGGGAAG | CGTGAACGGCAAACCGAACCTGAAGCTGAACCCGCCG |
| | CTGGGAGAATACGCTGATGAAAGAGCGGTTTC | AACGCGTTCCTGAGCATTAACGAAATGCCGAAAGTGGT |
| | GAAAAGACTTGGTATGGAGTTTCAGTTATTCAA | TCTGCTGGAGATCCTGCAGCGTGGCAAGGTGACCGAAA |
| | TCCGCATTATCAGATTGAGAATAATAAAATTGG | TCATTAAAGAGTTTATCCAAGCGAGCACCGACAAGATT |
| | ATTTTCTTTTAGCCCAATAGAATGTTCTATAAAA | CTGAACCGTGAGTTCATCGAGGAAGTTAAGAGCAAACT |
| | AATGTTAATGGTAAGCCGAATTTGAAATTAAAT | GGATTTCAAAAAGCCGTTTAACCGTAGCTTCAGCAAAA |
| | CCACCGAATGCATTTTTAAGTATTAATGAAATG | AGCGTAACAGCGCGTATGGTCCGAAGGGCCTGCAGATT |
| | CCGAAAGTAGTTCTTCTGGAGATTTTACAGAGA | CTGACCGAACGTCGTACCAGCCTGAACCTGATCCTGAA |
| | GGAAAAGTAACGGAGATTATAAAGGAATTCAT | GGAGCACAACCTGAACGACAAACAAATTCCGGGTCGT |
| | TCAAGCAAGCACGGATAAAATACTGAATAGAG | ATCCTGGACTACTGGATGAACATCGTGGATGTTACCGA |
| | AATTTATTGAGGAAGTAAAGAGTAAATTGGATT | CGATAAAGCGATTGCGAACCGTATCCAGGCGATGAAA |
| | TTAAAAAACCATTTAACAGGAGTTTTAGCAAGA | AAGGACTGCCGTGATCGTCTGAAGCAAAAAGCGAAGA |
| | AAAGGAATTCTGCTTATGGACCTAAAGGACTGC | ACAAAGCGCCGAAGATTGGCGAAATGGCGACCTTCCTG |
| | AAATATTAACCGAAAGAAGAACTTCTCTAAATT | GCGCGTGACATTGTGGATATGGTTATCGACGAGAACGT |
| | TAATTTTAAAAGAACATAATCTGAATGACAAAC | TAAAAAGAAAATCACCAGCTTTTACTACGACAAAATGC |
| | AGATACCCGGAAGAATATTGGATTACTGGATGA | AGGAATGCCTGGCGCTGTATGGTGATGCGGAAAAGAA |
| | ATATTGTTGATGTGACGGATGATAAGGCAATAG | AGAGCTGTTTATCCGTATTTGCGGCGAGGAACTGAACC |
| | CCAATAGAATTCAGGCGATGAAAAAGGATTGC | TGTTCGATAAGGGTATTGGCCACCCGTTCCTGTTTGAGC |
| | AGAGACAGGCTTAAACAAAAAGCTAAAAACAA | TGAACCTGCAAAGCATCAACAAGACCAGCGAACTGTAC |
| | AGCACCAAAGATTGGAGAGATGGCAACGTTTCT | GAGAAATATCTGATCAAGAAGGGCACCGCGGAACACA |
| | TGCAAGAGATATTGTAGATATGGTGATTGATGA | TCAAGTGGAACGAGCGTACCAAGAAAAACTACAAAGT |
| | AAATGTAAAGAAAAAGATAACATCATTTTACTA | GGAAACCAGCTGGCTGTACACCAACTTCTACAACAAGA |
| | TGATAAGATGCAGGAATGCCTGGCGCTTTACGG | TCTGGAACGAGGAAAAGAAAAAGATGGAAACCAAGCT |
| | AGATGCAGAAAAGAAGGAGTTGTTTATAAGGA | GAAACTGCCGGAGGACCTGAGCAAGCTGCCGTTTAGCA |
| | TTTGCGGAGAGGAATTAAATCTTTTTGATAAGG | TCCGTAACCTGACCAAGGAGAAAAGCAGCCTGGATAA |
| | GAATAGGACATCCGTTTTTATTTGAGCTTAATTT | GTGGCTGAACAACGTTACCAAAGGCTGCCTGGAAAAA |
| | GCAAAGTATAAATAAGACATCGGAATTGTATG | GACCGTACCAAGCCGATTGATCTGCCGACCAACATCTT |
| | AGAAATATTTGATTAAAAAAGGAACGGCTGAG | CGACGAAACCCTGGTGAAAATCATTCGTGAGAAACTGA |
| | CATATTAAATGGAATGAAAGGACAAAGAAGAA | ACGATAAGCAGGTTAGCTACAAAGACACCGATAAATAT |
| | TTATAAAGTTGAAACATCGTGGCTATATACAAA | AGCAAGCTGCTGGAGCTGTGGAAGGGTGGCGACACCC |
| | TTTTTATAACAAGATTTGGAATGAAGAGAAAAA | AACCGTTCTATAACGCGGAGCGTGAGTACACCGTGTAT |
| | GAAAATGGAAACGAAGCTAAAACTTCCTGAGG | GAGGAAAAAGTTCGTTTTCGTCTGGGCGAGAAGAACAG |
| | ATTTATCAAAATTACCGTTTTCGATTCGCAACCT | CTTTAAGGAGTACTTCAAAGATGCGCTGGAAAAGGTGT |
| | TACTAAAGAAAAGTCTTCGCTTGATAAATGGCT | TCAAAAAGGAGAGCAGCAAGCGTCAGAGCGAACGTGG |
| | AAACAATGTGACGAAAGGATGCTTAGAAAAAG | CAAACCGCCGATTCAGAAGAAGGACCTGCTGACCGTTT |
| | ATAGGACGAAGCCAATTGATTTGCCGACAAAC | TTAACGATGCGATCACCGAAAACGAGAAGGTGGTTCGT |
| | ATATTTGATGAAACATTAGTTAAGATAATAAGA | TTCTATCAGACCAAAGACCGTGTGATGCTGATGATGGT |
| | GAAAAACTAAATGATAAACAAGTATCGTATAA | TAAGGACCTGATGGGTGCGGAGCTGGATTTCAAACTGA |
| | GGATACGGATAAATATTCAAAATTGCTGGAGTT | GCGAAATCTACCCGCTGAGCGAGAAGAGCCCGCTGAA |
| | ATGGAAGGGTGGAGATACACAGCCGTTTTACA | CATTGAGGAAGAGATCGAACAACGTGTGGAGGGCAAA |
| | ATGCGGAGCGAGAATACACTGTTTATGAAGAG | CTGAGCTACGACGGTGATGGCAACTATATTAAAGGTGG |
| | AAGGTGCGATTTAGATTGGGTGAAAAAAATTCA | CAAGGAAAGCATCACCAAGATCATTTACGCGCGTCGTA |
| | TTTAAAGAATATTTTAAGGATGCTTTAGAGAAA | AGCGTAAAGACTTCACCGTTTTTAAAAAGCTGACCTTT |
| | GTTTTTAAAAAAGAATCTTCAAAAAGGCAGAGC | GATAAACGTCTGCCGGAACTGTTCGAGTACTATGCGGA |
| | GAACGAGGGAAGCCACCGATACAAAAGAAAGA | AGAGCGTATCCCGTACGAGAAGCTGAAAGCGGAACTG |
| | TTTGCTGACGGTTTTTAACGATGCCATAACAGA | GACGAGTATAACAAACACCGTGACATGGTGTTTGATGT |
| | AAACGAAAAGGTGGTGCGTTTTTATCAGACGAA | GGTTTTCGAACTGGAGAAAAAGATCATGGATAAGCCGG |
| | GGATAGGGTGATGCTGATGATGGTAAAGGATTT | AAGCGCTGCGTGAAATGGAGGACGTGGGTGATAAGAA |
| | AATGGGAGCGGAACTTGATTTTAAATTAAGTGA | CGTTCGTCACAAACCGTACCTGAACTGGCTGAAAAAGC |
| | AATATATCCTTTGTCGGAAAAGAGTCCGCTAAA | GTAAAGTGATTGACAAAAAGCAGTATGCGCTGCTGAAC |
| | CATAGAGGAAGAAATAGAGCAAAGAGTGGAGG | GCGATCCGTAACAGCTTCAGCCACAACCAATACCCGCC |
| | GGAAATTAAGTTATGACGGGGATGGAAATTAT | GCGTATGATCGTTGAGAACAAGATCAAGATCAAGGCG |
| | ATAAAAGGGGGTAAGGAGAGTATTACGAAAAT | GGTGGCATTACCCCGCAGATCTTTGAACGTTACAAGGA |
| | AATTTATGCCAGAAGGAAGAGAAAAGATTTCA | AGAGATTGAGATCATTATGAACAAAATCTAG (SEQ ID |
| | CAGTGTTTAAGAAACTTACGTTTGATAAGCGAT | NO: 40) |
| | TGCCGGAATTGTTTGAGTATTATGCAGAAGAGA | |
| | GAATACCATACGAAAAACTTAAGGCAGAATTG | |
| | GACGAATACAACAAACACAGGGATATGGTATT | |
| | TGACGTGGTATTTGAACTGGAAAAGAAGATAAT | |
| | GGATAAGCCGGAAGCTTTGAGGGAAATGGAGG | |
| | ATGTGGGGGATAAAAATGTGCGACATAAACCA | |
| | TATTTGAACTGGTTGAAAAAAAGGAAAGTGAT | |
| | AGATAAAAAGCAGTATGCATTATTAAATGCGAT | |
| | AAGGAATTCATTTTCGCATAATCAGTATCCGCC | |
| | GAGAATGATAGTGGAAAATAAAATTAAGATAA | |
| | AAGCGGGAGGAATAACACCCCAAATATTTGAA | |
| | AGATATAAAGAAGAAATAGAGATAATAATGAA | |
| | TAAAATATAG (SEQ ID NO: 39) | |
| Type VI | ATGCGGATCATACGGCCCTACGGCACCAGCGCG | ATGCGTATCATTCGTCCGTACGGCACCAGCGCGACCGA |
| Cas_4 | ACCGAGCCGGACGCGCAGGACCCGGCCAAGCG | GCCGGATGCGCAGGACCCGGCGAAACGTCGTCGTACCC |
| | CCGGCGCACGCTGCGGCGCAAGCTCGACGCGC | TGCGTCGTAAGCTGGATGCGCCGGGTGCGACCACCGTT |
| | CGGGCGCGACAACGGTCACCGAGCGCGACCTC | ACCGAACGTGACCTGGGTGCGTTCGCGCGTCGTCACGA |
| | GGAGCGTTCGCCCGCCGCCACGACGTGCTGGTC | TGTGCTGGTTATTGGCCAGTGGATCAGCACCATTGATA |
| | ATCGGCCAGTGGATCTCGACGATCGACAAGATC | AAATCGCGAGCAAGCCGGCGGGTTTTAAAAAGCCGGG |
| | GCCAGCAAGCCCGCAGGCTTCAAGAAGCCCGG | TGCGGAGCAACGTGCGCTGCGTCGTCGTCTGGGTGAAG |
| | CGCCGAGCAGCGGGCGCTGCGGCGCAGGCTCG | CGGCGTGGCGTCATATTGTTGCGCACGGTCTGCTGCCG |
| | GCGAGGCCGCCTGGCGCCACATCGTGGCACAC | GGTCGTGCGGAAACCCCGAGCCTGGAAACCCTGTGGTG |
| | GGCCTCCTGCCCGGGCGCGCCGAGACCCCCTCG | GATGCGTCTGGAGCCGTACCCGACCGGTGACGCGAAAT |
| | CTCGAAACCCTGTGGTGGATGCGGCTCGAGCCC | ATGGCCGTGATCCGAAGGGTCGTTGGTACGCGCGTTTC |
| | TATCCGACGGGCGATGCCAAGTACGGGCGCGA | GTGGGCGAGATTGAACCGGAGGAAATCGACGCGGATG |
| | TCCCAAAGGACGCTGGTACGCGCGCTTCGTCGG | CGGTGGTTGAGCGTATTGCGGAACACCTGTATGCGCAC |
| | CGAGATCGAGCCCGAGGAGATCGACGCCGATG | GAACATCCGATTCATCCGGGCCTGCCGACCCGTCGTGA |
| | CGGTCGTCGAGCGCATCGCCGAGCACCTCTACG | AGGTCGTATTGCGCATCGTGCGGCGAGCATCCAGGCGG |
| | CGCACGAGCACCCGATCCACCCGGGCCTGCCGA | CGGTTCCGAAAGCGGAGCCGCGTGCGGCGCGTGCGACC |
| | CGCGCCGCGAGGGACGGATCGCGCATCGCGCC | TGGACCGACGCGCACTGGACCATTTACGCGGAAGCGGG |
| | GCCTCGATCCAGGCTGCCGTGCCGAAGGCGGA | CGATGTTGCGGCGGTTATCCGTGCTGCGGCGGAGGAAG |
| | ACCTCGTGCCGCGCGCGCGACGTGGACGGATGC | TGCAAGCTCCGCCGCCGCCGGATGATAAAGCGGCGAA |
| | GCACTGGACGATCTACGCCGAGGCCGGGGACG | GGGCAAGCGTCGTTGGGTGGGTCCGGATGTTGCGGGCA |
| | TGGCGGCGGTGATCCGTGCGGCGGCCGAAGAG | AGGCGCTGTTCGAGCACTGGCAACGTGTGTTTGTTGAT |
| | GTCCAGGCGCCGCCCCCGCCCGACGACAAGGC | CCGGAAACCGAAGCGGTGCTGAGCGTTGGCGAGGTGA |
| | GGCGAAGGGCAAGCGGCGCTGGGTCGGGCCCG | AGGCGCGTATCGAAAACGGTGACGATCGTCTGCGTGCG |
| | ACGTCGCCGGCAAGGCGCTGTTCGAGCACTGGC | CTGTTCGAACTGCACGAGGAAGTTCGTGGTGCGTACCG |
| | AGCGCGTGTTCGTCGATCCCGAGACCGAGGCCG | TCGTCTGCTGAAACGTCACCGCAAGGCGGTGCGTGGTA |
| | TCTTGAGCGTGGGCGAGGTCAAGGCGCGGATC | GCAGCGGCAAACCGACCCGTACCAGCGACGTTGCGCGT |
| | GAGAACGGCGACGACCGCCTGCGGGCGCTGTT | CTGCTGCCGAGCAGCATGGATGCGCTGCAGCGTCTGCT |
| | CGAGCTCCACGAAGAGGTCCGCGGCGCCTACC | GGCGGCGCAACGTGACAACCGTGATGTGAACGCGCTG |
| | GCCGGCTCCTCAAGCGTCACCGCAAAGCCGTGC | ATTCGTTTTGGCAAGGTTATCCACTATGAAGCGGCGGA |
| | GCGGATCCTCCGGTAAGCCGACCCGGACCAGC | ACCGACCAGCGAGGTGCCGCCGGATGATGATGGTCGTC |
| | GATGTCGCCCGTCTCCTACCGTCGTCGATGGAC | CGCGTCATGATGAACCGGCGCATGTGCTGGATGACTGG |
| | GCACTCCAGAGACTGCTTGCGGCGCAGCGCGAC | CCGGATGCGGCGCGTGTTGCGCGTAGCCGTTTCTGGAC |
| | AACCGCGACGTCAACGCCCTGATCCGGTTCGGC | CAGCGATGGTCAGGCGGAGATTAAAGCGAACGAAGCG |
| | AAGGTCATCCACTACGAGGCGGCCGAGCCGAC | TTTGTGCGTATCTGGCGTCGTGTTCTGGCGCTGATGCAC |
| | CTCCGAGGTTCCGCCGGACGACGACGGGCGAC | CGTACCGCGACCGATTGGGCGATGCCGGAGGCGGATG |
| | CGCGCCACGACGAGCCCGCGCACGTGCTCGAC | ACGATTTCACGATGGCGCGTGTGCTGGAGCGTGCGGTT |
| | GACTGGCCCGACGCCGCGCGGGTGGCCCGGAG | GGTGAAGACTTTGATCAAGCGCGTCACCGTCGTAAGGT |
| | CCGCTTCTGGACCAGCGACGGCCAGGCCGAGAT | TGAACTGCTGTTCGGTGCGCGTGCGGACCTGTTTCGTG |
| | CAAGGCCAACGAGGCCTTCGTGCGCATCTGGCG | GTGATGGTGCGGATGATGCGCTGGACCGTGAGGTGCTG |
| | TCGGGTGCTCGCGCTCATGCACCGCACGGCGAC | CGTTTCGCGCTGGAACACCTGCGTAGCCTGCGTAACAA |
| | GGACTGGGCGATGCCCGAGGCCGATGACGATTT | GAGCTTCCACTTTGTGGGTGTTGGTGGCTTTAAGGCGGT |
| | CACCATGGCGCGCGTGCTCGAGCGGGCCGTTGG | GCTGACCGGCGCGAACGAGGCGCCGGCGGATGGTGCG |
| | CGAAGACTTCGACCAGGCGCGGCATCGGCGCA | GCGCCGGCGCAAGCGCGTGCGCTGTGGGCGCAGGATC |
| | AGGTCGAGCTCCTGTTCGGTGCACGAGCCGACC | AACGTGAACGTGCGAAACAACTGGGCAAGGTGCTGCA |
| | TGTTCCGGGGTGACGGCGCCGACGACGCGCTCG | GGGTGTTCAAGCGGGCGACTACCTGGAGGGTAACGAA |
| | ATCGCGAGGTGCTGCGGTTCGCCCTCGAGCACC | CTGCGTGCGCTGTTCGACGATCTGGTTGCGGCGATGAC |
| | TGCGCAGCTTGCGCAACAAGTCCTTTCACTTCG | CACCCCGAGCGATCTGCCGCTGCCGCGTTTTAAACGTG |
| | TCGGCGTCGGCGGTTTCAAGGCAGTGTTGACCG | TTCTGCTGCGTGCGGAGAACATTCGTGACAAGCGTCAA |
| | GGGCCAACGAGGCGCCGGCCGACGGGGCTGCG | GATGATCCGCACCTGCCGGCGCCGGCGAACCGTCTGGA |
| | CCGGCACAGGCCCGGGCCCTCTGGGCGCAGGA | TCTGGAGGAACCGGCGCGTCTGTGCCAATACACCGCGC |
| | TCAGCGCGAGCGGGCCAAACAGCTCGGCAAGG | TGAAACTGGTTTATGAGCGTCCGTTTCGTCGTTGGCTGG |
| | TCCTGCAGGGCGTGCAGGCGGGGGACTACCTCG | CGGATGCGGATGCGGCGAAAGTGCGTGGTTATGTTGAG |
| | AGGGCAACGAGCTTCGAGCGCTCTTCGATGACC | GGTGCGGCGCGTCGTAGCACCGATGCGGCGCGTAAACT |
| | TCGTCGCGGCGATGACGACGCCTTCCGACCTGC | GAACGACCCGAAAGATGAGGCGAAGCGTGAACGTGTG |
| | CGCTGCCCCGCTTCAAGCGGGTGCTGCTCCGCG | CGTAGCAAGGCGGAACGTATTGCGAACCTGGCGCCGG |
| | CCGAGAACATCCGCGACAAGCGCCAAGACGAC | ATGCGACCATGCGTGATTTTGTGCGTACCCTGATGCGT |
| | CCGCACCTGCCCGCGCCCGCCAACCGTCTCGAC | GAAACCGCGAGCGAAATGCGTGTTCAGCGTGGCTACGA |
| | CTCGAGGAGCCAGCGCGCCTCTGTCAGTACACC | GAGCGACGCGGAAAACGCGCGTGATCAAGCGCGTTAT |
| | GCGCTCAAGCTCGTCTACGAACGACCGTTCCGC | ATTGAGGACCTGCTGCGTGATGTGGTTGCGCTGGCGTT |
| | CGCTGGCTCGCCGATGCCGACGCGGCCAAGGTC | CCTGGACTACTTTCGTGATGCGAAATTCGGTTTTCTGCT |
| | CGAGGCTATGTCGAGGGCGCCGCCCGGCGTTCG | GGAAATTGCGGCGGACCGTACCGTGGACCCGGCGAAA |
| | ACCGACGCGGCGCGCAAGCTCAACGACCCCAA | CGTCTGGACCCGACCACCCTGGAGGCGCCGGAAGCGG |
| | GGACGAGGCGAAACGCGAGCGCGTCCGCTCGA | ATGTGAGCGCGGAGCCGTGGCAGGTGGCGCTGTATTTC |
| | AGGCCGAGCGGATCGCGAACCTGGCGCCCGAC | GTTAGCCACCTGGCGCCGGTGGACGATATTGCGCTGCT |
| | GCGACCATGCGCGATTTCGTCAGGACGCTGATG | GCTGCACCAACTGCGTAAATTTGACATCCTGGCGGAGA |
| | CGTGAGACGGCGAGCGAGATGCGCGTGCAGCG | AGCGTGGTGCGGGCACCGATGATGCGCTGCGTGCGCAG |
| | CGGCTACGAGAGCGACGCCGAGAACGCCCGCG | GTTGAAGCGGTGATCAAAGTTTTCGACCTGTACCTGGA |
| | ACCAGGCGCGCTACATCGAGGACCTCCTGCGCG | CATGCACGATGCGAAGTTTGAGGGTGGCCGTGGTCTGG |
| | ACGTCGTGGCGCTGGCGTTCCTCGACTACTTCC | CGGGCCTGGAAGATTTCGCGCAGCTGTTTGAGAGC |
| | GGGACGCGAAGTTCGGATTCCTGCTCGAGATTG | CGTGAACTGTTCGAGGAACTGGTGGCGAAACCGGTTGG |
| | CCGCGGACCGCACGGTCGATCCGGCGAAGCGG | TCAAGACGATAGCGAGCGTGTGCCGGTTCGTGGCCTGC |
| | CTCGATCCGACCACGCTCGAAGCCCCCGAGGCC | GTGAAATTGCGCGT |
| | GACGTGTCGGCAGAACCCTGGCAGGTGGCGCTC | TATGGTCACCTGCCGCCGCTGCTGCCGATTTTCCAGAA |
| | TATTTCGTGAGCCATCTCGCACCGGTCGACGAC | ACGTCGTATCACCGAGGAAGACGCGCGTGAGTTTCGTG |
| | ATCGCGCTCCTCCTGCACCAGCTGCGCAAGTTC | AACGTGGTGGCACC |
| | GACATCCTCGCCGAGAAGCGCGGTGCGGGCAC | ATCGCGGATCGTCAGAAAGAGCGTCAAGCGCTGCATGC |
| | CGACGACGCGTTGCGCGCTCAGGTCGAGGCCGT | GGAGTGGGCGGAAAAGCCGAAAGCGTTCGCGAACCAC |
| | CATCAAGGTCTTCGATCTCTACCTCGACATGCA | AGCGTGGCGGAATAC |
| | CGACGCCAAGTTCGAGGGCGGACGCGGGCTCG | ACCCGTGCGCTGCGTGACGTTGCGCAACACCGTCATTG |
| | CCGGTCTGGAGGACTTCGCCCAACTCTTCGAGA | CGCGAACCATGTGAGCCTGACCGCGCACGTTCGTCTGC |
| | GCCGCGAGCTCTTCGAGGAGCTGGTCGCGAAGC | ACCGTCTGCTGATG |
| | CGGTGGGCCAGGACGACAGCGAACGCGTGCCG | GGTGTTCTGGGCCGTCTGCTGGACTTCAGCGGCCTGTTT |
| | GTGCGCGGCCTGCGCGAGATCGCCCGCTACGGG | GAGCGTGATCTGTACTTTGCGGCGCTGGCGCTGGTGCA |
| | CATCTGCCGCCGCTCCTGCCCATCTTCCAGAAG | TGAAAACGGCCTG |
| | CGCAGGATCACCGAGGAGGATGCCCGGGAGTT | CGTACCGAGGAAGCGTTTGGTAAACGTTGCGCGTATCT |
| | TCGCGAGCGCGGAGGCACGATCGCGGACCGGC | GATTGGTCAGGGCCGTATTCTGGCGGCGATCCGTCACC |
| | AGAAGGAGCGCCAGGCGCTGCACGCGGAATGG | TGGACGCGGAGATC |
| | GCGGAAAAGCCGAAAGCATTCGCTAACCACTC | CAAAAGGAACTGGGTGGCCTGTTCCTGCTGGATGGTGC |
| | GGTGGCGGAATACACCCGCGCCCTGCGAGACG | GACCAAAGTTATCCGTAACCACTTCGCGCACTTTAAGA |
| | TCGCGCAGCACCGTCATTGCGCCAATCACGTGA | TGCTGCAGCCGAGC |
| | GTCTCACGGCCCATGTGCGCCTGCATCGGCTGC | CGTGCGGATGCTGCGGCGCTGAACCTGACCAGCGAGGT |
| | TGATGGGCGTGCTCGGACGACTGTTGGACTTCT | GAACGGCTGCCGTCAACTGATGCGTTACGATCGTAAGC |
| | CGGGCCTGTTCGAGCGCGACCTCTACTTCGCCG | TGAAAAACGCGGTGACCAAAGCGGTTATTGAGTTTCTG |
| | CCTTGGCGCTCGTTCACGAGAACGGCTTGAGGA | GAGCGTGAAGGTCTGGACATCCGTTGGACCTGGAACGA |
| | CGGAGGAGGCGTTCGGCAAGCGTTGCGCCTATC | TGCGCACGAACTGAGCGTTCCGACCCTGAAAACCCGTG |
| | TGATTGGACAGGGACGGATCCTTGCTGCGATCC | CGGCGAAACATCTGGGTGGCCGTGCGATTGCGGAGCGT |
| | GACATTTGGATGCGGAGATTCAAAAAGAACTC | CGTGAAGATGGTGCGGTGCCGGACGTTCGTGATGGTTT |
| | GGCGGCCTGTTTCTTTTGGACGGCGCCACAAAG | TCCGATCCAGGAAGCGCTGCATGCGGCGGGCTATGTGG |
| | GTCATCCGGAACCACTTCGCCCACTTCAAAATG | AAATGACCGCGGCGCTGTTTGCGGGTCATGCGGCGCCG |
| | CTGCAACCTTCGAGGGCCGACGCGGCGGCGCTC | ATTCGTAACGAGATCTGCGCGCTGGACCTGGAACGTAT |
| | AACCTGACGAGCGAGGTCAACGGCTGCCGGCA | CGATTGGCGTCGTCCGCAGCGTCGTGACGGTAGCAAGG |
| | GCTGATGCGTTACGACCGCAAGCTCAAGAACGC | GTAAAGGCAAGGGTAAAGGCAAGAACCGTCACCCGGC |
| | GGTGACGAAAGCCGTCATCGAGTTCTTGGAACG | GCCGAACAAGGCGCAATAG (SEQ ID NO: 42) |
| | CGAGGGGCTCGACATCCGGTGGACCTGGAACG | |
| | ACGCGCACGAGCTGAGCGTGCCGACGCTCAAG | |
| | ACCCGCGCCGCCAAGCACCTCGGCGGCAGAGC | |
| | CATCGCCGAACGCCGTGAGGACGGCGCCGTGC | |
| | CCGACGTGAGGGATGGATTTCCGATCCAGGAG | |
| | GCGCTCCACGCCGCTGGCTACGTCGAGATGACA | |
| | GCCGCCCTGTTCGCCGGCCATGCGGCGCCCATC | |
| | CGCAACGAGATCTGCGCGCTGGATCTCGAGCGC | |
| | ATCGACTGGCGCCGGCCGCAGCGCAGGGACGG | |
| | CTCCAAGGGGAAGGGGAAAGGGAAAGGCAAG | |
| | AACCGGCACCCTGCGCCGAATAAGGCCCAGTA | |
| | G (SEQ ID NO: 41) | |
| Type VI | ATGCAAAAGCATCAAATAATGGATAAAGGCAA | ATGCAGAAACACCAAATCATGGATAAGGGTAACGCGG |
| Cas_5 | TGCAGAGGGCAATTACCGGCACTTTGATGAAGA | AGGGCAACTACCGTCACTTCGACGAGGAAGCGGATAA |
| | AGCCGATAAACCTTTTTATGCTGCTTACCTGAA | ACCGTTTTACGCGGCGTATCTGAACACCGCGAAGCAGA |
| | TACGGCCAAACAAAACATCTIITTAGTGCTCAG | ACATCTTTCTGGTGCTGCGTGACATTAGCGAGAAACTG |
| | GGACATTTCTGAAAAGCTGGACCTGGGTTTCAA | GATCTGGGTTTCAACTTTGACAGCGACGATCAGCTGTT |
| | TTTCGACAGTGATGATCAGCTATTTAGTGTGGA | CAGCGTTGAACTGTGGAAACAACTGAAGACCGGCAAA |
| | GCTGTGGAAACAGCTTAAAACCGGGAAAAGGC | CGTCCGAACCTGACCCAGAAAATCATTGCGCACCTGAA |
| | CTAATCTTACCCAGAAGATCATAGCGCATTTAA | GCAGCAACTGCCGTTCCTGGAAATCGCGGCGATTGCGA |
| | AACAGCAATTGCCGTTTTTAGAAATTGCAGCAA | ACGCGCGTAAACAGAGCAACGATCACAAGGCGCAGCC |
| | TTGCTAATGCCCGTAAACAATCCAATGACCATA | GCAACCGGAGGACTACTATCACATCCTGGAACACTGGG |
| | AAGCCCAACCTCAACCGGAGGACTACTATCACA | TGAGCCAACTGCTGGACTACTGCAACTACTATACCCAC |
| | TTTTAGAGCATTGGGTCAGCCAATTGCTTGATT | GCGACCCACAACAGCGTGAACATGGCGCGTGTTATCAT |
| | ACTGCAATTACTACACCCATGCCACACACAATT | TGGTGGCATGCTGGACGTGTTCGATAGCGCGCGTCGTC |
| | CGGTCAATATGGCTCGTGTGATCATTGGAGGAA | GTGTTAAAGATCGTTTTAGCCTGATGCCGGCGGATGTG |
| | TGCTTGATGTATTTGATTCGGCTCGCAGACGTG | GAGCACCTGGTTCGTCTGGGTCCGAAGGGTGGCCAGAA |
| | TGAAAGACCGTTTTTCCTTAATGCCCGCAGATG | CGATCGTTTCCACTACAGCTTTCTGGACAAACAAGGTC |
| | TAGAGCATTTGGTTAGGCTTGGGCCAAAGGGCG | GTCTGACCGAAAAGGGCTTCCTGTTCTTTACCAGCCTGT |
| | GGCAAAATGATCGTTTTCATTACAGTTTCCTGG | GGCTGAAGAAAAAGGATGCGCAGGAGTTCCTGAAAAA |
| | ATAAGCAAGGGCGCCTAACCGAAAAAGGATTT | GCACGAAGGTTTTAAACAGAGCCAAGAGAACGCGGAC |
| | TTATTCTTTACATCTCTTTGGCTTAAAAAAAAGG | AAGGCGACCCTGGAAGCGTTCACCATCTTTGGCATTAA |
| | ATGCCCAGGAATTTTTGAAAAAACATGAAGGAT | GCTGCCGAAACCGCGTCTGACCAGCGACCTGGGTGATC |
| | TTAAGCAAAGCCAGGAAAACGCTGATAAAGCT | AAGGCCTGTTTATGGACATGGTTAACGAACTGAAGCGT |
| | ACTTTAGAAGCCTTCACGATTTTCGGTATAAAG | TGCCCGGAGGAACTGTACAGCCTGCTGAGCAAAGAGG |
| | TTACCCAAGCCACGATTAACAAGCGATCTGGGT | ATCAGGCGACCTTCAAGCCGCACGACAGCGAGGAAGC |
| | GATCAGGGCTTATTCATGGATATGGTGAATGAG | GACCAACGACGATGAGAACCCGCCGGAACTGAAACGT |
| | CTTAAACGTTGTCCGGAAGAGCTTTATTCACTG | AACCAAAACCGTTTCTACTATTTTGCGCTGCGTTATCTG |
| | CTTAGCAAAGAAGACCAAGCCACATTTAAACC | GAGAACGCGTTCCAGAACCTGCGTTTTCAAATCGATCT |
| | GCATGATTCTGAAGAAGCAACAAATGATGATG | GGGTAACTACTGCTTCAAGACCTATGAGCAGGAAATCG |
| | AAAACCCACCTGAATTAAAGCGAAATCAGAAC | AGCAAGTGGCGTACAAACGTCGTTGGTTCAAGCGTATT |
| | CGGTTTTACTACTTTGCCTTGCGATACCTGGAA | ACCGCGTTTGGCCGTCTGACCGACTATAAAGAGCACAA |
| | AATGCCTTTCAGAACCTCAGGTTTCAAATTGAT | CCAGCCGATGGAATGGGAGGAAAAGCTGCTGAAAGTT |
| | CTGGGCAATTATTGCTTCAAAACTTATGAGCAA | CCGGACCGTGATAAGCCGGACACCTACATCACCGATAC |
| | GAGATAGAGCAGGTAGCGTACAAAAGACGGTG | CACCCCGCACTATCACCTGAACGAGAACAACATTGGTC |
| | GTTTAAACGAATAACCGCTTTTGGACGGTTGAC | TGAAAAAGGTGACCGACAAGGATAAAGTTTGGCCGGA |
| | AGATTACAAGGAGCATAACCAGCCAATGGAAT | GATCCCGAAAAAGGAAAACGGTAAAAAGCCGGAGGGT |
| | GGGAAGAAAAATTGCTAAAAGTTCCTGATAGG | AACCCGCCGGACTTCTGGCTGAGCATCTACGAACTGCC |
| | GACAAACCCGACACCTATATCACTGATACCACA | GGCGGTGGTGTTCTACCAGATTCTGTATGAGAAAGGTC |
| | CCGCATTACCATTTAAATGAAAACAACATCGGG | TGGCGCAATTCAGCGCGGAGAGCATCATTGAAATCTAC |
| | CTTAAAAAAGTAACGGATAAGGATAAAGTTTG | GCGGGCGAGATTCAGAAACTGCTGGACGATGTGAAGG |
| | GCCAGAAATTCCCAAAAAAGAAAATGGTAAAA | TTGGTAACATCGCGAGCGGCTATAGCAAGGAACAGCTG |
| | AACCGGAAGGTAATCCTCCCGATTTTTGGTTAA | CAAACCGAACTGGAGAACCGTGCGCTGCACATCAGCTA |
| | GTATTTACGAGCTGCCGGCAGTAGTTTTTTATC | CATTCCGAAACCGGTGATTAAGTATCTGCTGGGCGAAG |
| | AAATCCTTTATGAAAAAGGCTTAGCACAGTTTT | ATGAGTGGAGCTTTGAGGAAAAAGCTGCGGCGCGTCTG |
| | CAGCCGAAAGCATAATCGAAATATACGCCGGA | CAGGCGCTGAAGGCGGAGAACGACCAACTGCTGAAAA |
| | GAAATTCAAAAATTGCTGGATGACGTAAAAGTC | AGGTTAAGCGTAAACAGCTGCACTTCCGTCAAAAACCG |
| | GGAAACATTGCTTCCGGATATTCAAAGGAGCAA | AGCAACAAGGATTTTCGTATCATGAAACCGGAGGAAAT |
| | TTGCAAACAGAACTGGAAAACCGGGCTTTGCAC | TGCGGACTTCCTGGCGCGTGATATGATCTGGCTGCAGC |
| | ATTTCTTATATACCCAAACCGGTGATCAAATAC | AACCGGACAACAAGGAGAAAAACAAGCCGAACAAAAC |
| | CTTTTGGGAGAGGATGAATGGTCATTTGAAGAA | CGAGTTCCACCACCTGCAGGGCAAGCTGACCTACTTTC |
| | AAAGCGGCTGCCCGCCTGCAGGCGTTAAAGGCT | GTAAATATAAGATGACCCTGCTGAAAACCTTTCGTCGT |
| | GAAAACGACCAATTGCTAAAAAAAGTAAAGCG | TGCAACCTGGTGGATGCGCCGAACGCGCACCCGTTCCT |
| | AAAGCAGCTCCACTTTAGGCAAAAACCCAGCA | GAACCAAATTAACCTGCTGGCGTGCAAGGGCCTGCTGA |
| | ACAAAGATTTTAGGATCATGAAACCAGAGGAA | ACTTCTACGTTACCTATCTGGAGCACCGTAAAGCGTTTC |
| | ATAGCGGATTTCCTGGCCCGCGACATGATCTGG | TGGAGCAGTGCACCAAGGAACAAGATTACGCGGCGTA |
| | CTGCAACAACCTGATAATAAGGAAAAAAACAA | TCACTTTCTGAAAGTGAAGCGTGACAAAGATGCGATCG |
| | ACCCAATAAGACAGAATTTCATCATCTTCAAGG | CGACCCTGATTGAAAAGCAGCAAGACGCGGTTTGCAAC |
| | CAAACTTACTTATTTCAGGAAGTACAAAATGAC | CTGCCGCGTGGTCTGTTCAAACAGCCGATCATGGAGGC |
| | TTTACTGAAAACATTCAGGCGCTGTAACCTGGT | GCTGAAGAACAGCGATGAAACCCGTGGCCTGGCGGCG |
| | GGATGCCCCAAATGCACACCCTTTTCTTAACCA | AGCCTGGAAAAAATGGACCGTGCGAACGTGGCGTTCAT |
| | AATCAATTTATTGGCCTGCAAAGGCCTCCTGAA | CATTCAGAACTACTTTCACGAGGTTCAGCAAGACGATA |
| | CTTTTATGTAACCTACCTGGAGCACAGGAAGGC | ACCAAGCGTTCTACGACTATAAGCGTAGCTACGAACTG |
| | TTTCCTGGAGCAATGTACCAAAGAACAGGATTA | CTGAACAAACTGTATGATCAGCGTAAGACCAACGACCG |
| | TGCAGCCTATCACTTTTTAAAGGTAAAGAGGGA | TAGCCCGCTGCCGAGCGTGTTCTTTAGCACCCGTGAGC |
| | TAAGGATGCTATTGCTACATTGATCGAAAAACA | TGGAGGAGAAGAAGGACGAAATCCCGCAGAAACTGGC |
| | GCAGGATGCCGTTTGCAACCTGCCAAGAGGGTT | GGACAAGGTTCAAAGCCGTATCGAGAAAAACAGCATT |
| | GTTCAAGCAACCCATCATGGAGGCATTAAAAA | AAGGATGAAAAAGAGAAGGAACGTATCCAGCAAAAGT |
| | ATTCGGATGAAACCCGTGGGTTAGCAGCATCAC | ACCGTAAACGTTATAAGCAGTTTACCGAGAACGAAAAG |
| | TCGAAAAAATGGATAGGGCCAATGTGGCCTTCA | CAAATCCGTTTCTTTAAGACCTGCGACATGGTGCTGTTC |
| | TTATTCAAAATTACTTTCATGAAGTCCAGCAAG | CTGATGGCGGATCAGATGTACCGTAGCGGTGACCCGAT |
| | ATGACAACCAGGCGTTTTACGACTACAAAAGG | CGGCCTGCACGACAACAACGATAACACCGCGCAAGGT |
| | AGTTATGAATTACTTAATAAGCTATATGACCAG | ATTACCGGTATGGGCGAAGCGTATAAACTGAAGAACAT |
| | CGGAAAACAAACGACAGAAGCCCCTTGCCATC | CCGTCCGGATGCGGAGCGTAGCATTCTGAGCCACGAAA |
| | AGTCTTTTTTTCAACCCGGGAGCTGGAGGAGAA | CCCTGGTGAAAATCCCGGTTTACTTCAACAACGCGAGC |
| | AAAAGACGAGATCCCGCAAAAATTAGCAGATA | GAGAGCCGTAGCAAGACCATCGTGCGTGAACGTATGA |
| | AGGTGCAATCACGGATTGAAAAAAACAGTATT | AGATCAAGAACTACGGTGATTTCCGTGCGTTTCTGAAA |
| | AAAGACGAAAAAGAAAAGGAACGAATTCAGCA | GACCGTCGTCTGACCGGCCTGCTGCCGTACATCGAGGC |
| | AAAATACAGGAAGCGATACAAGCAATTCACTG | GGATGAAATTGTTTATGAGGCGCTGAAGACCGAGTTCG |
| | AAAATGAAAAGCAAATCCGGTTTTTTAAAACCT | AAGCGTTTCACGACGCGCGTATCGAGGTGTTTGAAAAA |
| | GTGACATGGTCCTGTTTTTAATGGCGGACCAAA | ATTCTGGAGTTCGAAAAGATCTTTCTGATTAAAGTTCGT |
| | TGTACCGCAGTGGAGACCCAATCGGATTGCATG | CCGAAGGCGAAAAAGAAACGTTACATCCCGCACGAAC |
| | ATAATAACGATAATACGGCCCAGGGAATAACA | TGCTGCTGCAGCAAAACGCGATTGACCTGCCGAGCTAT |
| | GGTATGGGGGAAGCATACAAGCTCAAGAACAT | CAGATCAAGAACATGATTGCGCTGCACCACAGCTTCAA |
| | CAGACCCGATGCAGAAAGGAGTATTCTGTCACA | CCACAACCAGTACCCGGATGCGAAACAATTCGGCGAGT |
| | TGAAACCCTTGTTAAAATTCCGGTTTATTTTAAT | ATATCGACGGCAGCAACTTTAACCAGCTGAAGCTGTAC |
| | AATGCAAGTGAAAGCCGCTCCAAAACCATTGTA | ACCGCGGATAACCAAGAAGTGATGGCGCACAGCATCA |
| | AGGGAGAGAATGAAAATTAAAAATTACGGGGA | TTGTTCAGCTGAAGAAACTGGCGCTGTGGTACTATGAC |
| | TTTCCGTGCTTTCCTGAAAGATAGAAGGCTAAC | AAAGCGATTAAGCTGACCAACGCGAGCTAG (SEQ ID |
| | CGGTTTGTTGCCTTACATTGAGGCAGATGAAAT | NO: 44) |
| | AGTATATGAGGCTTTGAAAACAGAATTTGAGGC | |
| | TTTTCATGATGCGCGGATTGAGGTTTTTGAAAA | |
| | AATCCTCGAATTTGAAAAAATATTTCTTATAAA | |
| | GGTTAGACCTAAAGCAAAAAAGAAGAGGTATA | |
| | TACCTCATGAATTACTGCTTCAACAAAACGCGA | |
| | TAGATTTGCCGTCTTATCAAATAAAGAACATGA | |
| | TCGCTTTACACCATTCTTTTAATCACAACCAATA | |
| | CCCGGATGCTAAACAATTTGGTGAATACATAGA | |
| | CGGAAGCAATTTTAACCAGTTAAAATTGTACAC | |
| | TGCTGATAACCAGGAAGTAATGGCCCATTCCAT | |
| | CATTGTGCAATTAAAAAAACTGGCGTTATGGTA | |
| | CTATGATAAAGCCATAAAACTGACAAATGCTTC | |
| | TTAG (SEQ ID NO: 43) | |
| Type VI | ATGACTTTACCAGATAAACAACAATCCACAATA | ATGACCCTGCCGGACAAACAGCAAAGCACCATCTACAG |
| Cas_6 | TATTCAATGGACAGATCAGAAGATAAATATTTT | CATGGACCGTAGCGAGGATAAGTACTTCTTTGCGCTGT |
| | TTTGCCCTGTATTTGAATATTGCACAGAATAAT | ATCTGAACATTGCGCAGAACAACGTGGACAAAGTTCTG |
| | GTGGATAAAGTTCTTAAAGAATTTGACAGTTGG | AAGGAGTTCGATAGCTGGTTTAACAGCCTGAACGAAAC |
| | TTTAATAGCCTGAATGAAACAAGCCAGGGAAA | CAGCCAGGGTAAATACAACAGCGCGCAGGCGAAGTGG |
| | ATATAATAGTGCACAGGCCAAATGGCTTGATAA | CTGGACAACCGTCTGCCGGGCAGCGACAGCGATGTGCT |
| | CAGATTACCGGGTTCTGATTCAGATGTTCTTGA | GGAGGCGAAAGAACGTCTGGTTTATCTGCGTCGTTTCT |
| | AGCCAAAGAAAGACTTGTGTATTTACGCAGGTT | TTCCGTTCATCGAAACCGAATTTACCACCAAAGAATAC |
| | TTTTCCTTTTATTGAAACTGAATTTACAACGAAA | CACGGTTATCGTGAGAAGCTGCTGATGCTGTTCGAACG |
| | GAATATCATGGATACAGGGAAAAACTCTTGATG | TCTGAACGATTTTCGTAACTTCTTTACCCACGTGCACTA |
| | TTATTTGAAAGATTGAATGACTTCAGAAATTTC | CGAACGTAACGAGCTGGAATTTAGCCGTAACAAGAAA |
| | TTTACACATGTTCATTACGAAAGGAATGAACTT | ATGTTCGAGTTTCTGAACGAGGTTAAGGAAATCGCGCT |
| | GAATTTTCCAGGAATAAAAAAATGTTTGAGTTC | GAACAAACTGAACCAGCACCCGTACTATCTGGACGATA |
| | TTAAATGAAGTCAAAGAAATTGCCTTAAATAAA | ACATTCTGAACCACCTGCACGACCCGGATCAGCGTTTC |
| | TTAAATCAGCATCCCTATTATTTAGATGATAAT | AACTTTCAAAAGGAGAACAACATCAAAGACGCGATTA |
| | ATTTTAAATCATCTGCATGATCCTGATCAGAGG | ACTTCTTTGTGTGCCTGTTCCTGGAAAACAAGCACGCG |
| | TTTAATTTTCAAAAAGAAAACAATATAAAAGAT | CACGAGTACCTGAAGAAACAGAAAGGTTATAAGAGCA |
| | GCAATAAACTTTTTTGTTTGTTTGTTTCTCGAAA | GCCACAACCCGGAACACCGTGCGACCCTGAAAACCTAC |
| | ACAAACATGCACATGAATATCTTAAAAAGCAA | ACCTTTTATAGCATCAAGCTGCCGCGTCCGGTTTTCGAG |
| | AAGGGATATAAAAGTTCTCATAATCCTGAGCAC | AGCCGTGACATGAAACTGCGTCTGATTCTGGATGCGCT |
| | AGAGCAACACTGAAGACGTATACTTTTTATAGC | GAACGAACTGAAGAAATGCCCGAAGCAACTGTACGAT |
| | ATAAAATTGCCTCGTCCTGTATTTGAAAGCAGA | CACCTGAGCGAGAAACACCAGAAGCTGTGCCAAGTGG |
| | GACATGAAGCTTAGGCTTATCCTTGATGCATTG | AAAGCGTTAAACAGAAGGAGAACGAGGAAAGCGGCGA |
| | AATGAACTGAAAAAATGTCCTAAACAATTATAC | AACCGAGGAAATCAAAGAGTATATCCCGTTCATTCGTC |
| | GATCATTTATCGGAAAAACACCAAAAGCTTTGC | ACGAAGACAAGTTTCCGTACTATGCGCTGCGTTTCATT |
| | CAGGTTGAATCTGTAAAACAAAAAGAAAATGA | GACGATCTGGAGCTGCTGAAAGACATCCGTTTCAAAAT |
| | GGAATCTGGAGAAACAGAAGAAATTAAGGAGT | TAAGCGTGGTCTGGGCAAGGAGTTCTTCCACACCCACG |
| | ATATACCCTTTATTCGACATGAAGATAAGTTTC | AAACCGCGACCCAGCCGGTGGTTCGTAACAAGAAAGT |
| | CTTATTATGCTCTTCGATTCATTGATGACCTGGA | GTTCACCTTTCGTCGTTTTCTGGAAGTTTACGAGGGTGA |
| | ATTACTCAAAGATATTCGTTTTAAAATCAAACG | ACGTAAAGAACCGGACAACAACCTGTGGCACCCGGCG |
| | GGGATTGGGAAAAGAATTTTTTCACACTCATGA | CCGGCGTATGCGTTCGAGAAAGATGGCAACATCAAAGT |
| | AACTGCAACTCAACCGGTTGTTAGAAATAAAAA | GAAGATTACCAAGAACGAGGAAACCAGCAAAAGCAAG |
| | AGTCTTTACTTTCAGAAGATTCCTGGAGGTTTAT | GACGATACCAGCAGCGACGACATCGCGTACGCGGAAC |
| | GAGGGAGAAAGAAAAGAACCCGATAATAACCT | TGAGCGTGTATGAGCTGCGTAACCTGGTTTACTGCTGC |
| | ATGGCATCCTGCTCCGGCTTATGCCTTTGAGAA | CTGAACGGTAAGAAAGACGCGGCGAACAACATCATCC |
| | AGATGGAAACATCAAAGTTAAGATAACAAAAA | GTGATTACGTTTTCAACTACAAAGCGTTTCTGAAGGAC |
| | ATGAAGAAACATCGAAATCAAAAGATGATACT | CTGGAAAACAAGGATTTCAGCGAGATCGACGATTACAC |
| | TCAAGTGATGATATTGCCTACGCAGAGCTGAGC | CGCGCAACTGGAGGAGCGTAAGCAGCAACTGCAGAAC |
| | GTTTATGAATTAAGAAATCTCGTTTATTGTTGCC | AAACTGAGCGAATATAACCTGCAGCTGCACCAACTGCC |
| | TGAATGGCAAAAAAGATGCAGCAAATAATATC | GAAGAAAATCCGTAAAATTCTGCTGGACGAGAAGATTC |
| | ATCAGGGATTATGTTTTCAACTATAAAGCTTTTT | AGGATTACAAAAGCCACACCATCCAAAAAATTAAGGA |
| | TAAAAGATTTAGAAAACAAGGATTTTTCAGAAA | CCGTCAGGAAGAGAACAAGCGTATCCTGGGTAAAATTA |
| | TTGATGATTATACAGCACAATTGGAAGAACGAA | AGGCGCAGAAACAAATGAGCAAGGAAAACGACAAAGA |
| | AACAACAACTCCAAAACAAATTATCTGAATATA | TAGCCAGCAAAAGAACACCCTGAAAACCGGTCAACTG |
| | ACCTACAATTGCATCAGCTTCCCAAAAAAATCA | GCGAGCGAGCTGGCGAACGACATCCAGAACTACCTGCC |
| | GAAAAATTTTACTGGATGAAAAAATCCAGGACT | GGAAAACTATAAACTGGAGCTGTTCCAATACCGTGATC |
| | ATAAGTCTCACACCATTCAAAAAATAAAGGAC | TGCAGAAACAACTGGCGTACTATCGTCGTAAGGAGATC |
| | AGGCAGGAAGAAAACAAACGTATTCTGGGAAA | TATATTCTGCTGAACCAGAACTACGCGCTGACCTATCA |
| | AATCAAAGCTCAGAAACAAATGAGCAAAGAAA | CGAACAGCAAGACCGTAACGAGAACTTCAACGATCTGT |
| | ACGACAAAGATAGTCAACAAAAAAATACTCTA | ACTACAAGAAAAAGCACCCGTTCCTGCACCACGTGCTG |
| | AAAACCGGCCAATTGGCAAGCGAATTAGCCAA | ACCCGTAAAGACAACGACGACATCTTCAGCTTTGCGTT |
| | TGATATTCAAAACTATCTGCCTGAGAATTACAA | CAACTACTTCAAAAGCAAGGAAATTTGGCTGGAGAAA |
| | ACTGGAACTATTTCAATACAGGGATTTGCAAAA | GTGCGTAAAAAGGTTATCGGCCTGAACGACACCGATAT |
| | ACAATTGGCTTATTACAGGAGAAAGGAAATAT | TCCGAAGTACAGCGAACTGTTTTACTACTTCAAGCCGG |
| | ATATATTACTCAATCAAAATTATGCATTGACTT | GCACCAGCGTGAACGAGAAAGGCGAAAAGATCTACTA |
| | ACCATGAACAGCAAGACAGGAATGAAAATTTT | TCGTAAGTACGACGATCACTATCTGAACAAACTGATTC |
| | AATGATTTGTATTATAAAAAGAAACATCCTTTC | AGCGTCACCTGAAGCAAGACCACGTTATCAACATTCCG |
| | TTACACCACGTGTTGACACGAAAAGATAACGAT | CGTGGTATCCTGAACCAATTCATTTGCCCGGAGAAGGA |
| | GATATCTTTTCTTTTGCATTCAACTATTTTAAAT | AAGCTACGAGCAGAAAAACAACCCGATCCAGAAGATT |
| | CTAAAGAGATATGGCTGGAAAAAGTCCGTAAA | GCGGACCAATATCCGAGCACCCAGGATTTTTACAAATT |
| | AAAGTAATTGGGCTTAATGACACTGATATTCCA | CCCGCGTTTTTATCACCCGACCGGCGAAGTGCTGACCG |
| | AAATATTCCGAACTTTTTTATTATTTTAAACCGG | TTGAGGACATCAACTACAAACTGGTGGAGCTGAGCAAA |
| | GCACCTCAGTAAATGAAAAGGGAGAAAAAATT | GACAAGGATCACCCGCACAACAACGATAAAAAGGAGC |
| | TACTACCGCAAATACGATGACCACTATTTAAAT | ACAAAAAGGCGTACAACCAACTGAAAAAGTACCTGAA |
| | AAACTCATTCAAAGACACTTAAAACAAGATCAC | AAAGGAAAAGACCATCCGTTACATTCAGAGCTGCGACC |
| | GTTATCAATATTCCCCGGGGCATATTAAATCAG | GTGTTCTGCTGGAGATGATCAAGTACTACCTGAACAAC |
| | TTCATCTGCCCGGAGAAAGAATCATATGAACAA | TACTTCAAAAAGAGCAACGAGGAGTTCGAACTGGACCT |
| | AAAAACAATCCTATTCAAAAAATCGCAGATCA | GACCGATATTGAGCTGCGTGACCTGTTTAAATACGATG |
| | ATATCCTTCCACACAGGATTTTTATAAATTTCCT | AAACCAACGAAAGCATCCACAACAAGCTGGATCAAAA |
| | CGTTTTTATCATCCAACAGGTGAAGTATTAACC | AATGATTACCCTGAAGTTTCACCTGAACGGTCAGAGCT |
| | GTGGAAGATATTAACTATAAACTGGTAGAATTA | TCCTGGCGGAAGACAAACTGAACAACTTCGGCAAGCTG |
| | AGTAAAGATAAAGATCATCCACACAACAATGA | CACCGTTACATCTATGATGAGCGTTTCATCAGCATCTTC |
| | CAAAAAAGAGCATAAAAAAGCATACAACCAGC | AAGTACAAGGGTAACAAAGCGTTTGAAGGCGTTAAGA |
| | TTAAAAAATATCTTAAAAAAGAAAAGACTATA | CCGAGAGCATCTATAGCCAACTGGAAAAAATTCTGGAG |
| | CGATATATTCAGTCCTGTGACCGTGTTTTATTGG | GCGTTCGCGAAGGAGCAGCTGGAACTGTTCGAGTACGT |
| | AAATGATTAAATATTATCTGAATAATTATTTTA | GCAGCAATTTGAAAAAACCATCACCACCAACTTTGAGA |
| | AAAAGTCTAATGAGGAGTTTGAACTTGATTTAA | ACAAGGTTAACCAGAAACGTACCGAGGAAAACGCGCG |
| | CAGATATTGAGTTACGGGATTTATTTAAATATG | TCGTGAGAAGAACGGCAAGCCGCTGATTAGCGAGCACT |
| | ATGAAACCAATGAATCCATCCATAACAAACTGG | ACTTCCCGATCAGCATTCTGCTGAGCCTGACCGAGGAA |
| | ATCAGAAAATGATTACATTGAAATTCCATTTGA | TGGGGTTTTATCAGCGGCAAAAACCGTAACTTCATTAA |
| | ATGGGCAATCTTTTCTTGCAGAAGACAAACTCA | CACCGCGCGTAACAGCGCGGCGCACAACAAGCTGGAC |
| | ACAATTTTGGGAAACTCCATCGTTATATTTATG | GATAAATACATCGAAATGCTGAAGGACCGTGAGTACG |
| | ACGAAAGATTTATAAGTATTTTTAAATACAAAG | AAAACGATTATTTTGGCGCGGCGAGCAAAATCTTCAAC |
| | GGAACAAAGCATTTGAAGGAGTCAAAACAGAA | GACCTGACCGAGAAGATTCGTACCGCGTAG (SEQ ID |
| | AGCATCTATAGTCAATTGGAAAAAATTTTAGAA | NO: 46) |
| | GCTTTTGCCAAAGAACAACTGGAATTATTTGAA | |
| | TATGTGCAGCAATTTGAAAAAACGATAACAACT | |
| | AATTTTGAAAATAAAGTAAATCAAAAAAGAAC | |
| | AGAAGAAAATGCAAGGCGGGAAAAAAATGGG | |
| | AAACCGTTAATCTCAGAACATTACTTTCCGATT | |
| | TCAATATTACTTTCACTGACAGAGGAATGGGGC | |
| | TTTATTTCCGGAAAAAACCGAAATTTCATCAAT | |
| | ACAGCCCGCAACAGTGCTGCACATAATAAACTG | |
| | GATGATAAATACATTGAAATGCTTAAAGATAGA | |
| | GAATATGAAAATGATTATTTTGGGGCAGCCTCA | |
| | AAAATTTTTAATGACCTTACGGAAAAAATCAGA | |
| | ACTGCATAG (SEQ ID NO: 45) | |
| Type VI | ATGACTACAATAGAAAACTTTAGAAAATACAA | ATGACCACCATCGAGAACTTCCGTAAGTATAACGCGGA |
| Cas_7 | CGCCGATAAATCGTTTAAAAATATTTTCGATTT | CAAGAGCTTCAAGAACATCTTCGATTTCAAGGGCGAGA |
| | CAAAGGTGAGATTGCTCCTATAGCAGAAAAATC | TCGCGCCGATTGCGGAAAAGAGCAGCCGTAACCTGGA |
| | GTCGAGAAACCTTGAACTAAAGCTCAAAAACA | GCTGAAACTGAAGAACAAAGTGGGTGTTGAAACCAGC |
| | AAGTAGGCGTAGAAACATCGGTACATTATTTTG | GTGCACTACTTCGCGATCGGCCACGCGTTTAAGCAGAT |
| | CCATAGGGCATGCTTTCAAACAAATAGACAAA | TGATAAAGAAGCGGTTTTCGACTACATCTATGATGAGG |
| | GAAGCGGTATTTGATTATATTTATGATGAAGAA | AAACCGACAGCAAGAAACCGCACCGTTTTACCAGCCTG |
| | ACCGACTCAAAAAAACCTCATCGGTTTACTTCG | AAGCAGTTCGACGAGCAATTCTGCAAGGAACTGAAAA |
| | CTCAAACAGTTTGATGAGCAATTTTGCAAAGAA | ACATCGTGAGCACCATCCGTAACATTAACAGCCACTAT |
| | TTAAAAAATATAGTTTCAACCATTAGAAATATT | ATCCACGATTTCGGCCAGATTAAATGCGACACCCTGAG |
| | AACTCCCATTATATTCACGACTTTGGGCAAATA | CCTGCAACTGATTACCTTCCTGAAGGAGAGCTTTGAAC |
| | AAATGCGATACACTTTCTCTACAATTAATTACA | TGGCGGTGATCCAGACCTACCTGAAGAGCAAAGAGAG |
| | TTTCTTAAAGAAAGTTTCGAGTTAGCGGTTATT | CACCAAAGATGCGATGACCACCCAAGACTTCTTTGATG |
| | CAGACGTATTTGAAATCAAAAGAAAGTACAAA | CGCCGGACAAAGATAAGAAAATTGTTGAGTTCCTGAAG |
| | AGATGCTATGACTACCCAAGATTTTTTTGATGC | GAACGTTTTTACGCGATCGACAGCGAGAAGAAAAACCT |
| | TCCCGATAAGGATAAAAAAATAGTTGAATTTCT | GGAAAGCTACCAGAACCACATCAACCGTAGCAAATATT |
| | TAAAGAAAGGTTTTATGCTATTGATTCTGAAAA | TCGGTACCCTGACCAAGGAGCAAGCGATCGAAACCATT |
| | GAAAAACTTAGAAAGCTATCAAAACCATATTA | CTGTTTGGCGAGGTGGTTGACCCGAACTTCAAGTGGAA |
| | ATCGTTCAAAATATTTTGGCACACTTACAAAAG | ACTGAACGAAACCCACATCGCGTTCCCGATTAGCGTTG |
| | AACAGGCTATTGAAACCATTCTCTTTGGCGAGG | GTAAATACCTGAGCTATCACGCGTGCCTGTTCATGCTG |
| | TGGTAGATCCTAATTTTAAATGGAAGTTGAACG | AGCATGTTTCTGTACAAGCACGAGGCGGAACAGCTGAT |
| | AGACACATATAGCTTTTCCTATTTCTGTCGGAA | CAGCAAGATTAAAGGCTTCAAGAAAAGCAAAAACGAC |
| | AATATCTTTCCTATCATGCCTGTTTATTCATGCT | GAGGATAAGCTGAAACGTAACATCTTCACCTTCTTTAG |
| | CAGTATGTTTCTGTACAAGCACGAGGCGGAGCA | CAAGAAATTCAGCAGCGAGGACATCAAAAGCGAACAG |
| | ATTGATTTCTAAAATAAAAGGGTTCAAGAAGTC | GCGCACCTGGTGAAGTTCCGTGACATTGTTCAATACCT |
| | GAAAAATGATGAAGATAAACTCAAACGCAATA | GAACCACTATCCGCTGGATTGGAACAAATACATCGAGC |
| | TTTTCACCTTTTTCTCAAAGAAATTCAGTAGCGA | TGGAAAGCGCGTATCCGAGCATGACCGACAAGCTGAA |
| | AGATATTAAAAGCGAACAAGCTCATTTGGTAAA | AGCGAAGATCATTGAGATGGAAATTGATCGTAGCTACC |
| | GTTTCGAGATATTGTTCAATACCTCAACCATTA | CGAACTTCGTGGGTAACACCCGTTTTCACACCTATATCA |
| | CCCATTGGATTGGAATAAATATATAGAATTGGA | AGTTCGAGCTGTGGGGTAAGAAATTCTTTGGCAACAAG |
| | ATCAGCTTACCCCTCAATGACTGATAAACTGAA | ATCTTCAAAGAATATTGCGACTGCAGCTTCACCCCGAA |
| | AGCTAAGATTATTGAAATGGAAATTGATCGTTC | AGAGCTGGAGGAATTTAAGTACGAAAAAGATACCTGC |
| | TTATCCAAATTTTGTAGGAAATACAAGATTTCA | GGCAAAGTTAAGGACGCGGAGCTGAAACTGAAGGAAA |
| | TACTTATATAAAATTTGAGTTATGGGGAAAAAA | AACACCTGCTGAAACACGATGAGATCAAGAAACTGGA |
| | ATTCTTTGGAAATAAAATTTTTAAAGAATATTG | AGACAAGATTGAGGAAAACAAGGATAAACCGAACAAC |
| | CGATTGTTCTTTTACCCCAAAGGAATTAGAAGA | ATTACCCTGACCCTGGATACCCGTATCAAGAAAAACCT |
| | ATTCAAATATGAAAAAGATACTTGCGGAAAAG | GCTGTTCACCAGCTATGGTCGTAACCAGGACCGTTTCA |
| | TAAAAGATGCGGAATTAAAATTAAAAGAAAAA | TGCAATTTGCGACCCGTTACCTGGCGGAGACCAACTAT |
| | CATCTATTAAAACATGATGAAATAAAAAAACTT | TTTGGCAAGGACGCGCAGTTCAAAATGTACCGTTTCTTT |
| | GAAGATAAAATAGAGGAAAACAAAGACAAGCC | AGCAGCGTGGATAACACCAACGAGATTGAAAGCCAGA |
| | CAACAATATTACTTTAACCCTCGATACCCGAAT | AGGAAAAACTGGACAAGAAACTGATCAACAAGAAACA |
| | TAAAAAAAACCTCTTGTTCACATCTTACGGGCG | ATTCGATAACCTGCGTTTTCACGACGGTCGTCTGACCTA |
| | AAATCAAGACCGATTTATGCAATTTGCCACTCG | CTTCGCGACCTTTAAGGAGCACCTGGTGCGTTATGAAA |
| | CTATTTAGCAGAAACGAACTACTTTGGCAAGGA | ACTGGGATACCCCGTTCGTTGAGGAAAACAACGCGGTG |
| | TGCACAATTCAAGATGTACCGATTCTTTTCATC | CAGGTTCAAATCACCTTTAACTACGAGGAAATTCTGAA |
| | GGTAGATAATACCAATGAAATTGAATCTCAAAA | AGACACCAACCAGACCATCCTGGTGTATATTACCAAGG |
| | AGAGAAGCTAGATAAAAAACTGATTAATAAAA | TTATCAGCATTCAACGTAGCCTGATGGTTTACTTCCTGG |
| | AACAATTTGACAACCTCAGATTTCACGACGGCA | AGGATGCGCTGAAAAGCAACACCCTGGCGAACAGCGA |
| | GACTCACTTACTTCGCAACATTTAAAGAACATC | AGGTGTGGGCGTTAAGCTGCTGTTCAACTACTATATGC |
| | TGGTGCGTTACGAAAACTGGGATACGCCGTTTG | ACCACAAGAAAGAGTTTGCGGAAAACAAACACGAGCT |
| | TAGAGGAAAACAATGCGGTACAGGTTCAAATC | GGAAAACAACGATAAGGAGAGCATCGACAACACCTAC |
| | ACATTTAATTATGAAGAAATACTTAAAGATACA | AAGAAAATCTTCCCGAAGCGTCTGATTAACAAATTTGT |
| | AATCAAACAATTTTAGTTTACATAACGAAAGTA | GGCGGTTAGCCCGAACGACCCGAAACAGCAAAGCGTG |
| | ATATCTATTCAGAGAAGCTTAATGGTTTACTTTC | TATGAGAGCATCCTGGAAAAGGCGAAGAAAAGCGAGG |
| | TTGAAGATGCACTAAAATCAAACACATTGGCAA | AACGTTACAAGGACCTGCGTGCGAAAGCGGAGAAGGA |
| | ATTCGGAAGGAGTAGGGGTAAAATTGTTGTTTA | TAAACGTCTGGAAGACTTCGATAAACGTAACAAGGGTA |
| | ATTATTATATGCATCACAAAAAGGAATTTGCGG | AACAGTTCAAACTGCAATTTGTTCGTAAGGCGTGGCAC |
| | AGAATAAACATGAACTTGAAAACAACGATAAA | CTGATGTACTTTCGTGACATCTACAACCTGTATGCGATT |
| | GAAAGTATTGATAATACTTACAAGAAAATATTC | GATGGCAAACCGGAGAACCACCACAAGCACCTGCACA |
| | CCAAAACGATTGATTAATAAGTTTGTTGCAGTT | TCACCCGTGAGGAATTCAACAACTTTTGCCGTTACATGT |
| | AGCCCAAATGACCCAAAACAGCAATCTGTTTAT | TCGCGTTTGATGAAGTGCCGCAGTATAAGCTGCTGCTG |
| | GAAAGTATACTAGAAAAGGCAAAGAAATCGGA | AAAAACATGCTGGCGGAGAAACACTTCCTGGACAACA |
| | AGAGAGATATAAAGACCTACGTGCGAAAGCAG | AGGCGTTCGAAACCCTGTTTGATAGCAGCCACGACCTG |
| | AAAAAGACAAACGATTAGAAGATTTCGATAAA | AACAGCATGTATTGCAAGACCAAAGAGAAGTTTAAAGT |
| | AGAAACAAAGGGAAACAGTTCAAGTTACAGTT | TTGGATGAGCCAACCGAAAGAGACCAGCAACGACAAG |
| | CGTTCGCAAGGCATGGCACCTCATGTACTTCAG | GAACACTACACCCTGGCGAACTACGAAAAGTTCTTTAA |
| | AGATATATACAATTTATATGCTATTGACGGGAA | GGACAAGATGTTCTACATCAACCTGAGCCACTTCCGTG |
| | ACCCGAAAATCACCATAAACATTTACACATAAC | ATTTTCTGAAAGAGAAGAAACGTTTCATCATTGCGAAC |
| | TCGCGAAGAATTTAATAATTTTTGCCGTTATAT | GATAAGATCGTGTTTAAAAGCCTGGAAAACAACCAGTA |
| | GTTTGCTTTCGATGAAGTGCCGCAATACAAACT | TCTGATGCAAGACTACTATATTGAGGAAACCCCGGCGA |
| | ACTGCTTAAAAACATGCTCGCAGAAAAACATTT | AGGAGAAATACAAGACCAAAGAGGAATATAAGGCGAA |
| | TTTGGACAACAAGGCGTTTGAAACCCTGTTCGA | CAAAAACCTGTACAACGAACTGCGTAAGAGCCGTCTGG |
| | TAGCAGCCATGATTTGAATTCTATGTATTGCAA | AGGATGCGCTGCTGTACGAAATGGCGATGCACTATCTG |
| | AACCAAAGAAAAGTTTAAAGTTTGGATGAGCC | GGTATGGAGAAAGACATTACCAAGAACGCGAAAGTGC |
| | AACCCAAGGAAACCAGCAATGATAAAGAACAT | CGGTTCAGAAGATCCTGAGCCAAGACGTGAGCTTCGAA |
| | TATACCCTTGCCAATTATGAAAAGTTTTTCAAA | ATCAAGGATCTGAAAAACATTACCAACTACACCCTGAG |
| | GACAAAATGTTTTACATAAATCTCTCGCATTTC | CGTTCCGTTCAAGAAACTGGAGAGCTATCTGGGTCTGA |
| | AGAGATTTCCTCAAAGAGAAAAAAAGGTTTAT | TGGCGTTTAAGGAAAAACAGGAGCAAGAATACAAAGG |
| | AATAGCAAATGATAAGATTGTTTTCAAATCGCT | CAGCTATATGATTAACCTGGTGGAGTACCTGAAGAAAA |
| | TGAAAACAACCAGTATCTGATGCAAGACTACTA | TCGAACAGGACAAAGATACCAAGAAAGAGATCAAGCA |
| | TATAGAAGAAACACCAGCAAAAGAAAAGTATA | AATTTGGAACGATATCAACGGCAACAAGAAACTGAGC |
| | AGACAAAAGAAGAATACAAGGCAAACAAGAAT | CTGGATCAGCTGAACAAATTCGACGCGCACATCATTAG |
| | TTGTATAACGAACTACGCAAAAGCAGACTTGAA | CAACAGCATCAAGTTTACCCGTGTGGCGATCCTGTTCG |
| | GATGCATTGCTCTATGAGATGGCAATGCACTAC | AACAATACTTCATCGTTAAGCACAACCACAGCATCATT |
| | CTCGGCATGGAGAAAGATATTACAAAAAATGC | AAGGACAACCGTATCAGCTTCGAGGAAATCGAGGAAA |
| | AAAAGTTCCTGTTCAAAAAATTCTATCTCAAGA | TCAAGGAGTACTTCGTTAAGCTGACCCGTAACAAGGCG |
| | TGTATCATTTGAAATTAAAGACTTAAAAAACAT | TTCCACTTTAACATCCCGGAAAAGCCGTACAGCAGCCT |
| | TACCAACTACACCTTATCCGTCCCTTTTAAGAA | GCTGAAGGAGATCGAAAAACGTTTCATCCAGAAAGAG |
| | ATTGGAATCCTATTTAGGTTTGATGGCATTTAA | GTGAAGATCCAAAACCCGAAAAGCTTTGATGAGATTAA |
| | GGAAAAACAAGAACAGGAATATAAAGGAAGCT | GCTGAACGAAAAATACATCTGCAGCGCGTTCCTGAACA |
| | ATATGATTAATCTTGTTGAATATTTAAAGAAAA | GCCTGTACGACGTTTACTTCAACTTCAAGGAGAAGGAC |
| | TTGAACAAGATAAAGACACAAAAAAAGAAATA | GAAAAGAAAAAGCGTTACGATGCGGAACAGAAGTATT |
| | AAACAAATATGGAATGACATAAATGGAAATAA | TTACCGCGATCATTGCGTAG (SEQ ID NO: 48) |
| | AAAGCTTTCGCTCGACCAACTCAATAAATTTGA | |
| | TGCTCATATAATATCAAACTCCATTAAATTTAC | |
| | CAGAGTTGCTATTCTTTTTGAACAATATTTTATC | |
| | GTTAAGCATAATCATAGCATAATAAAAGACAA | |
| | CAGAATTTCTTTTGAAGAAATTGAAGAAATTAA | |
| | GGAATATTTTGTAAAACTCACCCGAAACAAAGC | |
| | ATTTCATTTTAACATTCCAGAAAAGCCTTATTCG | |
| | TCATTATTAAAAGAAATTGAAAAGAGATTTATT | |
| | CAAAAAGAAGTAAAGATTCAGAATCCTAAAAG | |
| | TTTCGATGAAATAAAGCTTAATGAAAAGTATAT | |
| | CTGCTCAGCATTTCTTAATTCTTTATATGATGTA | |
| | TATTTCAATTTTAAAGAAAAAGATGAAAAGAA | |
| | AAAACGGTACGATGCAGAACAGAAATATTTTA | |
| | CTGCGATAATTGCATAA (SEQ ID NO: 47) | |
| Type VI | ATGGAAACTACACAAACATCTGAAAACAAGAG | ATGGAAACCACCCAAACCAGCGAGAACAAACGTCGTA |
| Cas_8 | AAGGTCACTTGCAACTGACCCTCAGTATTTTGG | GCCTGGCGACCGATCCGCAGTACTTCGGTGGCTATCTG |
| | CGGCTATTTGAATATGGCACGGCTAAATATTTA | AACATGGCGCGTCTGAACATCTACAACATTAACAACTA |
| | TAACATTAATAATTATCTGGCGGAGGAGTTTGG | TCTGGCGGAGGAATTCGGCCTGAGCCAACTGCCGGAGG |
| | ACTTTCCCAACTCCCGGAAGATGGATATATTAA | ACGGTTACATCAAGAACAGCTTTCTGTGCAACCAGAAG |
| | AAACAGTTTTTTATGTAACCAAAAACAAACAAA | CAAACCAAACTGAACTGGAACCGTGTTTTCAGCAAAGC |
| | ACTTAACTGGAACCGGGTTTTTTCAAAGGCAGT | GGTGACCTTTCTGCCGATTCTGAAGGTTTTCGATAGCGA |
| | AACTTTTTTACCCATCCTGAAGGTTTTTGATTCT | AAGCCTGCCGAAGAGCGAAAAAGAGGACAAGAGCACC |
| | GAGTCACTACCGAAATCGGAAAAAGAAGATAA | CCGGAGACCGGCAAGGATTTTGCGAAAATGGCGGACA |
| | ATCAACACCCGAAACCGGCAAGGATTTCGCAA | GCCTGAAAGTGCTGTTCAGCGAAATCCAGGAGTTTCGT |
| | AAATGGCAGATTCCCTGAAAGTTCTCTTTTCCG | AACGATTATAGCCACTACTATAGCACCGAAAAGGGCAC |
| | AAATTCAGGAGTTCAGAAATGATTATTCTCATT | CGATCGTAAAATCACCATTAGCAACGAGCTGGCGGACT |
| | ACTACTCTACCGAAAAAGGCACTGATAGGAAA | TCCTGAAGTTTAACTACAAACGTGCGATCGAGTATACC |
| | ATTACCATTTCAAATGAACTGGCTGATTTTCTCA | CGTGTTCGTTTCAAGGACGTGTACACCGACGATGACTT |
| | AGTTTAATTACAAAAGAGCCATTGAATATACAA | TAACGTTGCGGCGAACAAGAAAATGGTTATCGGTGGCG |
| | GGGTGAGATTTAAAGATGTGTACACCGACGATG | TGATTACCACCGAAGGTCTGGTGTTCCTGACCAGCATG |
| | ATTTTAATGTGGCTGCTAATAAAAAAATGGTAA | TTTCTGGAGCGTGAGTACGCGTTCCAATTTATCGGCAA |
| | TCGGCGGGGTTATTACCACCGAAGGACTGGTTT | GATTACCGGCCTGAAAGGTACCCAGTATGTTGGTTTCC |
| | TTCTAACTTCCATGTTTCTTGAACGTGAATACGC | GTGCGTTTCGTGATGTGCTGATGGCGTTCTGCATCAAAC |
| | ATTTCAGTTTATCGGTAAAATTACAGGATTGAA | TGCCGCACGAGAAACTGAAGAGCGATGACTTCATTCAA |
| | GGGTACACAATATGTGGGTTTCAGGGCATTTCG | AGCTTTACCCTGGACATCATTAACGAACTGAACCGTTG |
| | AGATGTTTTAATGGCTTTTTGCATCAAACTTCCA | CCCGAAGACCCTGTACAACGTTATCACCGAGGAAGAGA |
| | CACGAAAAACTAAAAAGCGACGACTTTATCCA | AACGTAAATTCCGTCCGCAGATCGAACCGGAGAAGATT |
| | GTCGTTTACGCTCGACATAATTAATGAATTAAA | GATAACCTGCTGAAAAACAGCGGTATCGAACTGGAAG |
| | CCGTTGTCCAAAAACGCTTTACAATGTAATTAC | AGTACGACGAGAACTTTGATGACTATGTGGAAAGCCTG |
| | CGAAGAAGAAAAAAGGAAATTCAGACCGCAGA | ACCCGTAAAATTCGTCACGAGAACCGTTTCAACTACTT |
| | TTGAACCTGAAAAGATTGACAATTTACTGAAAA | TGCGCTGCGTTATATCGATGAGAACAAGATTTTCGGCA |
| | ACAGCGGGATTGAACTGGAAGAGTATGACGAA | AATACCGTTTTCAAATCGATCTGGGCAAGCTGGTTATC |
| | AATTTCGATGATTATGTGGAATCGTTGACCAGG | GACGAATACCCGAAGAAATTCTTTAACGAAGAGGTGCA |
| | AAAATACGTCACGAAAACAGGTTCAACTATTTT | GCGTCGTATCATTGAAAACGCGAAGGCGTTCGATAAAC |
| | GCATTACGTTATATTGACGAAAATAAAATTTTT | TGAGCGATCTGGTTGACGAGACCGCGATCCTGAAGAAA |
| | GGGAAATACCGTTTTCAAATCGATTTAGGAAAA | ATCGACATTCAGAACCACCAAGTGTACTTCGAACCGTT |
| | CTGGTGATTGATGAATATCCTAAAAAGTTCTTC | TGCGCCGCACTATAACACCGAGAACAACAAGATCGCGC |
| | AACGAAGAAGTTCAGCGGCGGATAATCGAAAA | TGCTGAGCAAAAGCGACATTGCGCGTGTTCGTAAAGTG |
| | TGCAAAAGCTTTTGACAAACTGAGTGATTTGGT | AAGACCAAAACCGGCGTTGAGCGTAAAAACCTGTTCCA |
| | TGATGAAACAGCGATTTTAAAGAAGATTGATAT | GCCGCTGCCGGAAGCGTTTCTGAGCTGCGCGGAGCTGT |
| | ACAAAACCACCAGGTTTATTTTGAACCTTTTGC | ACAAGATCGTTCTGCTGGAATATCTGAAGCCGGGTGAA |
| | ACCACATTACAATACCGAAAACAATAAAATTGC | GCGGAGAAACTGGTGACCGATTTCATTCTGGCGAACAA |
| | CTTATTATCAAAAAGTGATATTGCAAGAGTGCG | CAGCAAACTGATGAACATGCAGTTTATCGAGCTGGTTA |
| | AAAGGTAAAAACCAAAACAGGTGTAGAAAGAA | AGAAACAAATGCCGGGCTGGATTGTGTTCCAGAAGGA |
| | AAAACCTGTTTCAGCCTTTGCCTGAAGCTTTTTT | AACCGACACCAAAAGCCGTCTGGCGTATAGCCAAATCA |
| | GAGCTGTGCCGAATTGTATAAAATAGTGTTGCT | ACTTTAACGAACTGCTGAGCCGTAAGAGCCAGCTGAAC |
| | GGAATATTTAAAACCTGGTGAAGCTGAAAAACT | AAAGTTCTGGCGGAGCACAACCTGAACGATAAGCAGA |
| | GGTTACAGATTTTATTCTTGCCAACAACAGTAA | TCCCGAGCAAAATTCTGGAATTCTGGCTGAACATCAGC |
| | ACTGATGAATATGCAGTTTATTGAACTGGTGAA | GACGTGAAGCAGCAATTTACCACCGGCGAGCGTATCAA |
| | AAAACAAATGCCCGGTTGGATTGTATTTCAAAA | ACTGATTAAGCGTGACTGCATGAAACGTCTGAAGGCGC |
| | AGAAACCGATACAAAAAGCAGACTGGCTTATT | TGAAGAAATTCAAAACCACCGGCAAGGGCAAAATCCC |
| | CACAAATTAACTTTAATGAACTTTTAAGCAGAA | GAAGATTGGCGAGATGGCGACCTTTCTGGCGAAAGATA |
| | AAAGCCAATTGAATAAAGTATTAGCCGAACAC | TCGTTGACATGGTGATCGGCAAGGAAAAGAAACAAAA |
| | AATTTAAACGATAAACAAATTCCTTCAAAAATA | GATCACCAGCTTCTACTATGATAAGATGCAGGAATGCC |
| | TTGGAATTCTGGCTGAACATCAGTGATGTAAAA | TGGCGCTGTACGCGGACCCGGAGAAGAAAAAGACCTT |
| | CAACAGTTTACTACCGGGGAACGGATAAAACT | CATCCACATCATCACCCACGAACTGGGCCTGTACGAGA |
| | GATAAAGCGGGATTGTATGAAGCGGTTGAAAG | AAGATGGTCACCCGTTCCTGAACCGTATCAACTTTAAC |
| | CGCTTAAAAAATTCAAAACCACCGGAAAGGGA | GAGCTGCGTTATACCCGTGACATTTACGAAAAGTATCT |
| | AAAATCCCGAAAATTGGCGAAATGGCCACATTC | GGAAGAGAAAGGCGAGAAGATGGTTAAATTCTACAAC |
| | CTGGCAAAAGACATTGTTGACATGGTTATTGGA | GCGCGTCGTGGTAACTATACCGAAAAGGATAAAAGCTG |
| | AAAGAAAAGAAACAGAAAATAACTTCGTTTTA | GCTGCGTGAGACCTTTTATACCCTGGTGGAAAAGGAGA |
| | CTACGACAAAATGCAGGAATGTCTGGCCTTGTA | TCAAAGGTAAAAAGCGTATTATGACCGAGGTGGTTCTG |
| | TGCCGACCCTGAAAAAAAGAAAACATTTATTCA | CCGAGCGACAAGAGCAAAATCCCGTTCACCCTGCTGCA |
| | TATTATCACCCATGAACTTGGATTGTATGAAAA | ACTGGAAGAGAAAACCACCTACAGCCTGGCGGATTGG |
| | AGACGGCCACCCGTTTTTAAACCGCATAAATTT | CTGCAGAACATTACCAAGGGCAAAGAACACGGTGACG |
| | CAACGAATTGCGTTACACCCGCGATATTTATGA | GCAAAAAGCCGGTTAACCTGCCGACCAACCTGTTCGAT |
| | AAAATACCTCGAAGAAAAGGGAGAAAAAATGG | GAAACCATCACCAGCCTGCTGAAGACCGAGCTGGACA |
| | TGAAATTTTATAATGCCAGGCGAGGAAATTATA | ACAAACAGGCGCTGTACCCGGAAAACGCGAAGATGAA |
| | CGGAGAAAGATAAATCGTGGTTAAGGGAAACT | CGAGCTGTTCAAACTGTGGTGGATGGGTCGTGGCGATG |
| | TTTTACACTTTGGTGGAAAAAGAAATTAAAGGG | GTGTGCAACACTTTTACGACGCGGAGCGTGAGTATTTC |
| | AAAAAGAGGATAATGACCGAAGTGGTTTTACCT | GTTTTTGAGCAGCCGGTGAAGTTCAAACCGGGTAGCAA |
| | TCCGACAAATCAAAAATCCCATTCACGTTACTT | GGCGAAATTTAGCGACTACTATTGCATCGCGCTGACCA |
| | CAATTAGAAGAAAAAACAACGTATTCTTTGGCC | AAGCGTTCAAGGAAAAAGAGAAGACCGCGACCAAGGA |
| | GACTGGCTGCAAAACATTACCAAAGGAAAAGA | ACGTAAACAAGCGCCGGAGCTGGATGAAGTTGAGAAA |
| | GCACGGTGATGGAAAAAAACCGGTAAACCTTC | ACCTTTCAGCAAGCGATCGCGGGCACCGAAAAGGAGA |
| | CAACCAATCTTTTTGACGAAACAATTACCAGTT | TTCGTGAGCTGCAGGAAGAGGACCGTGTTTGCGCGCTG |
| | TGCTGAAGACAGAACTTGATAATAAACAGGCG | ATGCTGGAAAAGCTGATCAGCCGTGAGAAGCACATTAC |
| | CTTTACCCCGAAAATGCCAAAATGAACGAATTG | CGTGAAACTGGAAAGCATCGAGAACCTGCTGAAGGAA |
| | TTTAAACTTTGGTGGATGGGCCGTGGCGACGGG | AGCGTGGTTGTGAAACAAACCGTGAACGGCAAGCTGTA |
| | GTGCAACATTTTTATGACGCCGAAAGGGAATAT | CTTCGATGAAAACGGTAACGAGATTAAAGACAAGAGC |
| | TTTGTTTTTGAACAACCTGTAAAATTTAAACCC | AACCCGGTTATCACCAAAACCATTGTGGATAAGCGTAA |
| | GGCTCAAAGGCAAAATTCTCTGATTATTACTGC | GGGCAAAGACTACGGTCTGCTGCGTAAGTTTGCGAACG |
| | ATTGCGCTTACAAAAGCATTTAAGGAAAAGGA | ACCGTCGTGTTCCGGAACTGTTCGAGTATTTTAGCGGC |
| | GAAAACAGCTACAAAAGAGAGAAAACAGGCTC | GAAGAGATCCCGCTGGAACAGCTGAAAAAGGAGCTGG |
| | CTGAACTTGATGAAGTTGAAAAAACCTTTCAGC | ATGGTTACAACATTGCGAAACACCTGGTGTTCGACGTT |
| | AGGCAATTGCCGGAACTGAGAAAGAAATAAGG | GTGTTTCGTCTGGAAGAGAAGCTGATCAAAAGCAACCG |
| | GAATTACAGGAAGAAGACAGGGTTTGTGCGCTT | TAACGAGATCATTAGCTATTTCACCGATGACAAGGGCA |
| | ATGCTTGAAAAACTCATCAGCAGGGAAAAGCA | ACGCGAAAGGTGGCAACATTCAACACCTGCCGTACCTG |
| | TATTACCGTTAAATTGGAATCGATTGAGAATTT | AACCTGCTGAAGGAAAAAGATCTGGTTACCCCGGGCGA |
| | GTTAAAGGAATCAGTAGTTGTAAAACAAACCGT | GATGGCGTTCCTGAACATGGTGCGTAACTGCTTCAGCC |
| | TAATGGTAAACTGTATTTCGATGAAAACGGGAA | ACAACCAGTTTCCGAAAAAGAGCATCATGAAAAAGGTT |
| | CGAGATAAAAGACAAATCGAACCCAGTAATAA | GTGAAGCCGGGTGAAAACAACTTTGCGAAAAAGATCG |
| | CCAAAACCATTGTTGACAAACGGAAAGGAAAA | CGGACATTTACAACGAAAAAATCGAGGCGCTGATTCTG |
| | GATTACGGTTTACTCCGTAAATTTGCAAACGAC | AAGCTGGCGTAG (SEQ ID NO: 50) |
| | CGCCGTGTGCCCGAACTGTTTGAATATTTTTCCG | |
| | GCGAAGAAATACCGCTGGAACAGTTAAAAAAA | |
| | GAACTTGATGGGTACAACATTGCCAAACACCTG | |
| | GTTTTTGATGTTGTTTTCAGACTTGAGGAAAAA | |
| | CTGATTAAAAGTAACCGGAATGAAATTATTTCC | |
| | TATTTTACAGATGATAAAGGAAATGCAAAAGG | |
| | CGGAAACATACAGCACCTGCCTTATTTAAACCT | |
| | GCTGAAAGAAAAGGATTTGGTAACGCCCGGTG | |
| | AAATGGCTTTTTTGAACATGGTACGCAACTGTT | |
| | TTTCGCACAACCAGTTCCCGAAAAAGAGTATTA | |
| | TGAAAAAAGTTGTTAAGCCCGGTGAAAACAATT | |
| | TTGCAAAGAAAATTGCTGATATTTACAATGAAA | |
| | AAATTGAGGCTTTGATATTAAAACTTGCATAA | |
| | (SEQ ID NO: 49) | |

In some embodiments, the Type VI endonuclease of the disclosure is catalytically active.

In some embodiments, the Type VI endonuclease of the disclosure is catalytically dead, e.g. by introducing mutations in one or both of the HEPN domains.

The Type VI endonucleases of the disclosure can be modified to include an aptamer.

The Type VI endonuclease of the disclosure can be further fused to domains, e.g. catalytic domains to produce dual action Cas proteins. In some embodiments, a Type VI endonuclease is further fused to a base editor.

### Collateral Activity of Class 2 Type VI CRISPR-Cas RNA-Guided Endonucleases

In addition to the ability to cleave a target sequence in a ssRNA, the Type VI endonucleases of the disclosure also possess collateral (trans-cleavage activity), i.e. the ability to promiscuously cleave non-targeted DNA or RNA once activated by detection of a target DNA. Without being bound to any theory or mechanism, generally once a Type VI endonuclease of the disclosure is activated by the binding of a gRNA, which occurs when a sample includes a target sequence to which the gRNA hybridizes (i.e., the sample includes the targeted ssRNA), the Type VI endonuclease can become a nuclease that promiscuously cleaves oligonucleotides (ssRNAs) not comprising the target sequence of the gRNA (non-target oligonucleotides, to which the guide sequence of the gRNA does not hybridize). Thus, when the targeted ssRNA is present in the sample (e.g., in some embodiments above a threshold amount), the result can be cleavage of single stranded reporter oligonucleotides (e.g. labeled) in the sample, which can be detected using any convenient detection method.

Accordingly, provided herein are methods and compositions for detecting a target RNA in a sample. Also provided are methods and compositions for cleaving non-target RNA oligonucleotides, which can be utilized as detectors. These embodiments are described in further detail below.

gRNAs for Class 2 Type VI CRISPR-Cas RNA-Guided Endonucleases

The present disclosure provides RNA-targeting RNAs that direct the activities of the novel Type VI endonucleases of the disclosure to a specific target sequence within a target ssRNA. These RNA-targeting RNAs are also referred to herein as “gRNAs” or “gRNAs” Generally, as provided herein, a Type VI gRNA comprises a single segment comprising both a spacer (DNA-targeting sequence) and a Type VI “protein-binding sequence” together referred to as a crRNA. Also provided herein are nucleotide sequences encoding the Type VI gRNAs of the disclosure.

i. Spacer Sequences

The Type VI endonucleases of the disclosure are single crRNA-guided endonucleases (single guide RNA, sgRNA, while the Type II endonucleases of the disclosure are guided by a dual-RNA system consisting of a crRNA and a trans-activating crRNA (tracrRNA). The crRNA of the Type VI guides of the disclosure comprises a nucleotide sequence that is complementary to a sequence in a target RNA.

The crRNA portion of the Type VI gRNAs of the disclosure can have a length of from about 45 to about 70 nt. In some embodiments, the length can be about 60 to about 65 nt.

The RNA-targeting spacer sequence of a Type VI gRNA generally interacts with a target RNA in a sequence-specific manner via hybridization (i.e., base pairing). As such, the nucleotide sequence of the RNA-targeting sequence may vary and determines the location within the target RNA that the gRNA and the target RNA will interact. The RNA-targeting sequence of a subject Type VI gRNA can be modified (e.g., by genetic engineering) to hybridize to a desired sequence within a target RNA.

The RNA-targeting sequence of a subject Type VI gRNA can have a length of from about 18 nucleotides to about 30 nucleotides. For example, the length can be 27 nucleotides.

The percent complementarity between the RNA-targeting spacer sequence of the crRNA and the target sequence of the target RNA can be at least 60% (e.g., at least 65%, at least 70%, at least 75%, at least 80%, at least 85%, at least 90%, at least 95%, at least 97%, at least 98%, at least 99%, or 100%). In some embodiments, the percent complementarity between the RNA-targeting sequence of the crRNA-RNA and the target sequence of the target RNA is 100% over the 1-27 contiguous 5′-most nucleotides of the target sequence of the complementary strand of the target RNA. In some embodiments, the percent complementarity between the RNA-targeting sequence of the crRNA and the target sequence of the target RNA is at least 60% over about 1-27 contiguous nucleotides. In some embodiments, the percent complementarity between the RNA-targeting sequence of the crRNA and the target sequence of the target RNA is 100% over the 1-27 contiguous 5′-most nucleotides of the target sequence of the complementary strand of the target RNA and as low as 0% over the remainder. In such a case, the RNA-targeting sequence can be considered to be 1-27 nucleotides in length.

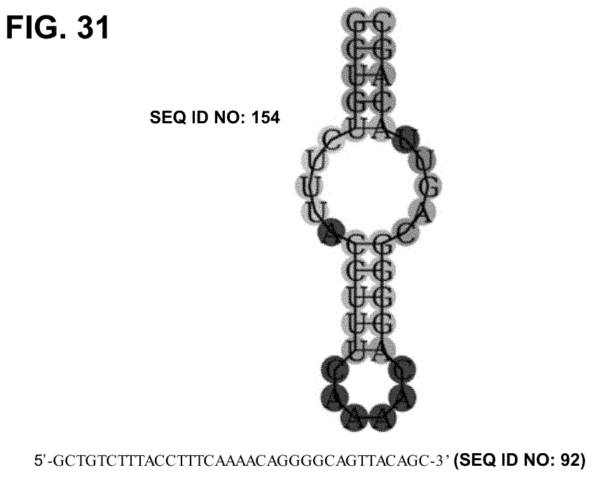

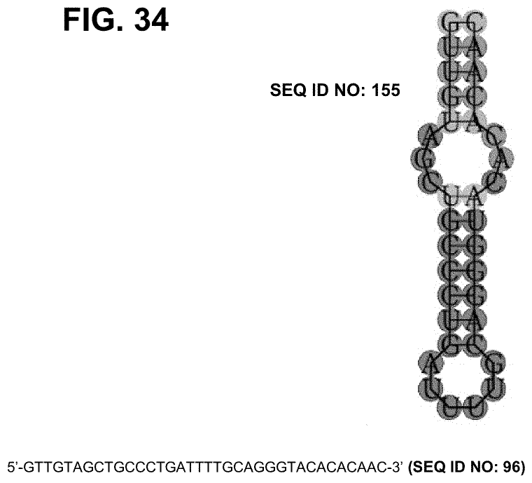

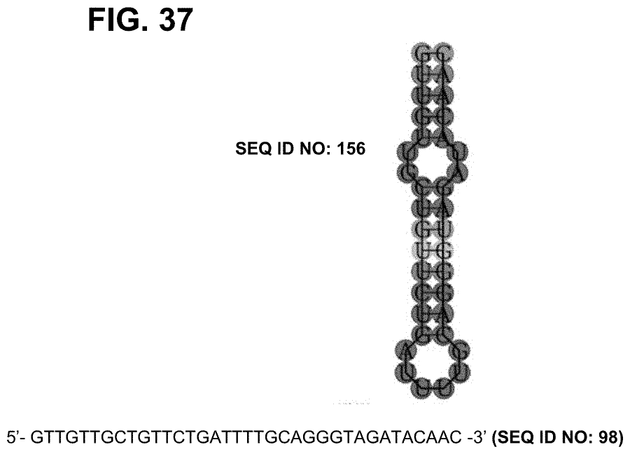

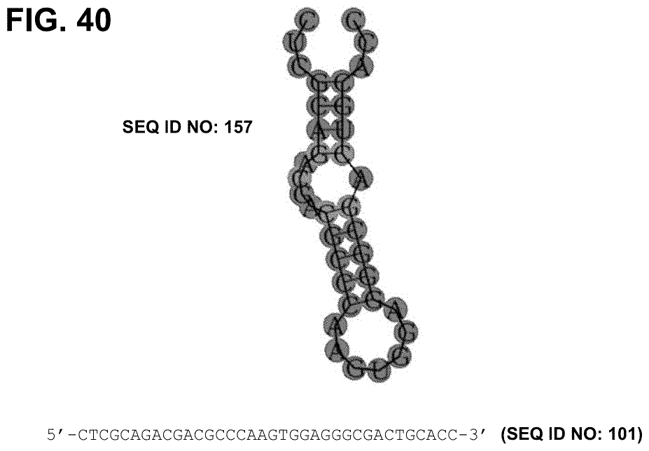

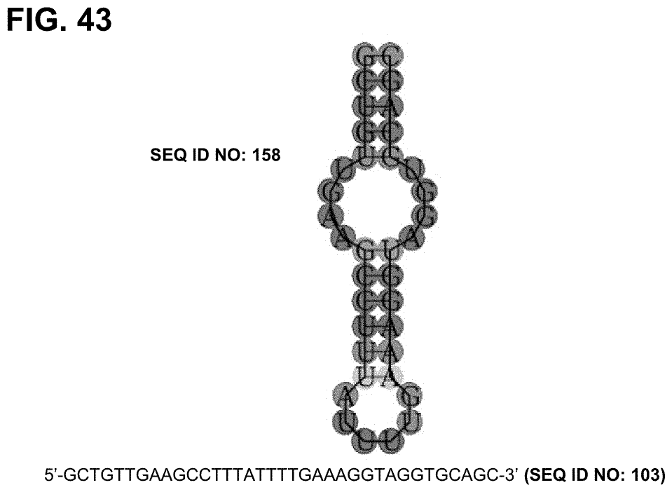

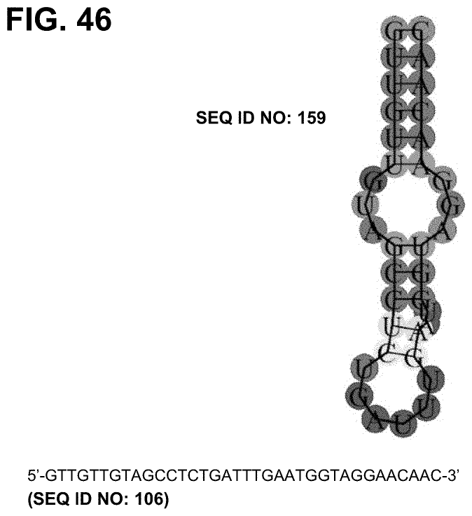

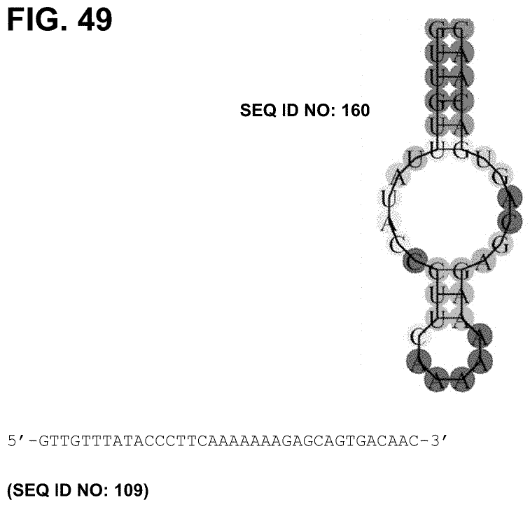

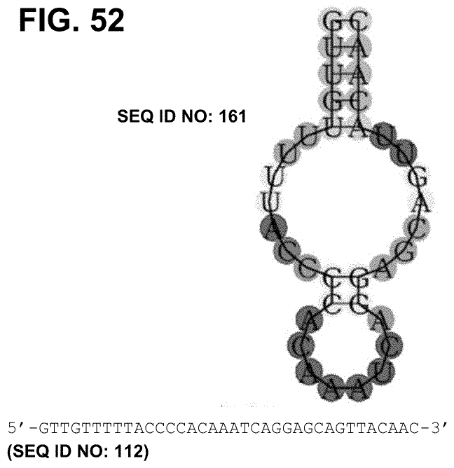

Generally, a naturally unprocessed pre-crRNA of Type VI comprises a direct repeat and an adjacent spacer (the portion of the crRNA that allows for targeting to a RNA molecule). In some embodiments, direct repeats (partial sequence or entire sequence) from unprocessed pre-crRNA are included into the Type VI gRNAs of the disclosure, and improve gRNA stability. Exemplary direct repeat sequences include SEQ ID NO: 92, 96, 98, 101, 103, 106, 109, and 112 (DNA sequences) or SEQ ID NOS 154-161 (RNA sequences). It is noted that while the exemplary sequences are provided in DNA nucleotides, it is understood that this DNA can then be transcribed into RNA. Accordingly the mature guides of disclosure may incorporate the entire or partial sequence of the exemplary direct repeat sequences provided herein; the guides may be composed of DNA nucleotides, analogous RNA nucleotides, or a combination of DNA and RNA nucleotides. Exemplary predicted secondary structures of the pre-crRNAs of the Type VI endonucleases of the disclosure are presented in FIGS. 31, 34, 37, 40, 43, 46, 49, and 52.

In some embodiments, the crRNAs include non-naturally occurring, engineered direct repeat sequences.

In some embodiments the spacer sequence of a Type VI gRNA of the disclosure is directed to a target sequence in a mammalian organism. In some embodiments the spacer sequence is directed to a target sequence in a non-mammalian organism.

In some embodiments, the spacer sequence of a Type VI gRNA of the disclosure is directed to a target sequence which is a sequence of a human. In some embodiments, the target sequence is a sequence of a non-human primate.

In some embodiments, the spacer sequence of a Type VI gRNA of the disclosure is directed to a target sequence in a mammalian organism, e.g. a human or non-human primate.

In some embodiments, the spacer sequence of a Type VI gRNA of the disclosure is directed to a target sequence in a bacteria.

In some embodiments, the spacer sequence of a Type VI gRNA of the disclosure is directed to a target sequence in a virus.

In some embodiments, the spacer sequence of a Type VI gRNA of the disclosure is directed to a target sequence in a plant.

The Type VI gRNAs of the disclosure can be modified to include an aptamer.

ii. gRNA Arrays

In some embodiments, the Type VI gRNAs of the disclosure can be provided as gRNA arrays.

Such gRNA arrays of the disclosure include more than one gRNA arrayed in tandem, and can be processed into two or more individual gRNAs. Thus, in some embodiments a precursor Type VI gRNA array comprises two or more (e.g., 3 or more, 4 or more, 5 or more, 2, 3, 4, or 5) gRNAs (e.g., arrayed in tandem as precursor molecules). In some embodiments, two or more gRNAs can be present on an array (a precursor gRNA array). A Type VI endonuclease of the disclosure can cleave the precursor gRNA array into individual gRNAs.

In some embodiments a Type VI gRNA array includes 2 or more gRNAs (e.g., 3 or more, 4 or more, 5 or more, 6 or more, or 7 or more, gRNAs). The gRNAs of a given array can target (i.e., can include guide sequences that hybridize to) different target sites of the same target RNA. In some embodiments, two or more gRNAs of a precursor gRNA array have the same guide sequence. In some embodiments, the precursor gRNA array comprises two or more gRNAs that target different target sites within the same target RNA. In some embodiments, the precursor gRNA array comprises two or more gRNAs that target different target RNAs.

### III. Class 2 Type II CRISPR-Cas RNA-Guided Systems

Provided herein are novel Class 2 Type II CRISPR-Cas RNA-guided proteins and their guide RNAs (a “guide RNA” is interchangeably referred to herein as “gRNA”), constituting the Class 2 Type II CRISPR-Cas RNA-guided systems of the disclosure. As used herein a gRNA may comprise only RNA nucleotides, may comprise RNA and DNA nucleotides, or may comprise only DNA nucleotides, and thus while referred to as a gRNA, may comprise non RNA-nucleotides.

Accordingly, provided herein are systems comprising (a) a Type II endonuclease, or a nucleic acid encoding the Type II endonuclease; and (b) a Type II gRNA, or a nucleic acid encoding the Type II gRNA, wherein the gRNA and the Type II endonuclease do not naturally occur together, wherein the gRNA is capable of hybridizing to a target sequence in a target DNA, and the gRNA is capable of forming a complex with the Type II endonuclease. It should be understood that

These components are described in turn below.

### Class 2 Type II CRISPR-Cas RNA-Guided Endonucleases

Provided herein are novel Type II CRISPR-Cas RNA-guided endonucleases. In some embodiments, these endonucleases may share certain structural, sequence, and/or functional similarities with any one of the subtypes of Cas9.

Without being bound to any theory or mechanism, a Type II CRISPR-Cas RNA-guided endonucleases of the disclosure comprise three RuvC motifs and a HNH domain, responsible for catalytic activity.

In some embodiments a Type II CRISPR-Cas RNA-guided endonuclease of the disclosure comprises any one of the RuvC sequences of Table 7, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type II CRISPR-Cas RNA-guided endonuclease of the disclosure comprises any two of the RuvC sequences of Table 7, or sequences comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type II CRISPR-Cas RNA-guided endonuclease of the disclosure comprises any three of the RuvC sequences of Table 7, or sequences comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type II CRISPR-Cas RNA-guided endonuclease of the disclosure comprises a HNH domain selected from the group consisting of SEQ ID NO: 138, SEQ ID NO: 139, SEQ ID NO: 140, and SEQ ID NO: 141, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type II CRISPR-Cas RNA-guided endonuclease of the disclosure comprises a RuvC I motif selected from the group consisting of SEQ ID NO: 116, SEQ ID NO: 121, SEQ ID NO: 126, and SEQ ID NO: 131, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type II CRISPR-Cas RNA-guided endonuclease of the disclosure comprises a RuvC II motif selected from the group consisting of SEQ ID NO: 117, SEQ ID NO: 122, SEQ ID NO: 127, and SEQ ID NO: 132, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type II CRISPR-Cas RNA-guided endonuclease of the disclosure comprises a RuvC III motif selected from the group consisting of SEQ ID NO: 118, SEQ ID NO: 123, SEQ ID NO: 128, and SEQ ID NO: 133, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type II CRISPR-Cas RNA-guided endonuclease of the disclosure comprises a (1) RuvC I motif selected from the group consisting of of SEQ ID NO: 116, SEQ ID NO: 121, SEQ ID NO: 126, and SEQ ID NO: 131, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; (2) a RuvC II motif selected from the group consisting of SEQ ID NO: 117, SEQ ID NO: 122, SEQ ID NO: 127, and SEQ ID NO: 132, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; and (3) a RuvC III motif selected from the group consisting of SEQ ID NO: 118, SEQ ID NO: 123, SEQ ID NO: 128, and SEQ ID NO: 133, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto. The Type II CRISPR-Cas RNA-guided endonuclease may further comprise a HNH domain selected from the group consisting of SEQ ID NO: 138, SEQ ID NO: 139, SEQ ID NO: 140, and SEQ ID NO: 141, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type II CRISPR-Cas RNA-guided endonuclease of the disclosure comprises a (1) RuvC I motif comprising the sequence of SEQ ID NO: 116, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; (2) a RuvC II motif comprising the sequence of SEQ ID NO: 117, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; and (3) a RuvC III motif comprising the sequence of SEQ ID NO: 118, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto. The Type II CRISPR-Cas RNA-guided endonuclease may further comprise a HNH domain selected from the group consisting of SEQ ID NO: 138, SEQ ID NO: 139, SEQ ID NO: 140, and SEQ ID NO: 141, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto. In some embodiments, the HNH domain comprises the sequence of SEQ ID NO: 138, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type II CRISPR-Cas RNA-guided endonuclease of the disclosure comprises a (1) RuvC I motif comprising the sequence of SEQ ID NO: 121, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; (2) a RuvC II motif comprising the sequence of SEQ ID NO: 122, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; and (3) a RuvC III motif comprising the sequence of SEQ ID NO: 123, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto. The Type II CRISPR-Cas RNA-guided endonuclease may further comprise a HNH domain selected from the group consisting of SEQ ID NO: 138, SEQ ID NO: 139, SEQ ID NO: 140, and SEQ ID NO: 141, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto. In some embodiments, the HNH domain comprises the sequence of SEQ ID NO: 139, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type II CRISPR-Cas RNA-guided endonuclease of the disclosure comprises a (1) RuvC I motif comprising the sequence of SEQ ID NO: 126, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; (2) a RuvC II motif comprising the sequence of SEQ ID NO: 127, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; and (3) a RuvC III motif comprising the sequence of SEQ ID NO: 128, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto. The Type II CRISPR-Cas RNA-guided endonuclease may further comprise a HNH domain selected from the group consisting of SEQ ID NO: 138, SEQ ID NO: 139, SEQ ID NO: 140, and SEQ ID NO: 141, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto. In some embodiments, the HNH domain comprises the sequence of SEQ ID NO: 140, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

In some embodiments a Type II CRISPR-Cas RNA-guided endonuclease of the disclosure comprises a (1) RuvC I motif comprising the sequence of SEQ ID NO: 131, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; (2) a RuvC II motif comprising the sequence of SEQ ID NO: 132, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto; and (3) a RuvC III motif comprising the sequence of SEQ ID NO: 133, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto. The Type II CRISPR-Cas RNA-guided endonuclease may further comprise a HNH domain selected from the group consisting of SEQ ID NO: 138, SEQ ID NO: 139, SEQ ID NO: 140, and SEQ ID NO: 141, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto. In some embodiments, the HNH domain comprises the sequence of SEQ ID NO: 141, or a sequence comprising at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

Table 7 provided exemplary RuvC I, RuvC II, RuvC III, and HNH domain sequences of the Type II endonucleases of the disclosure.

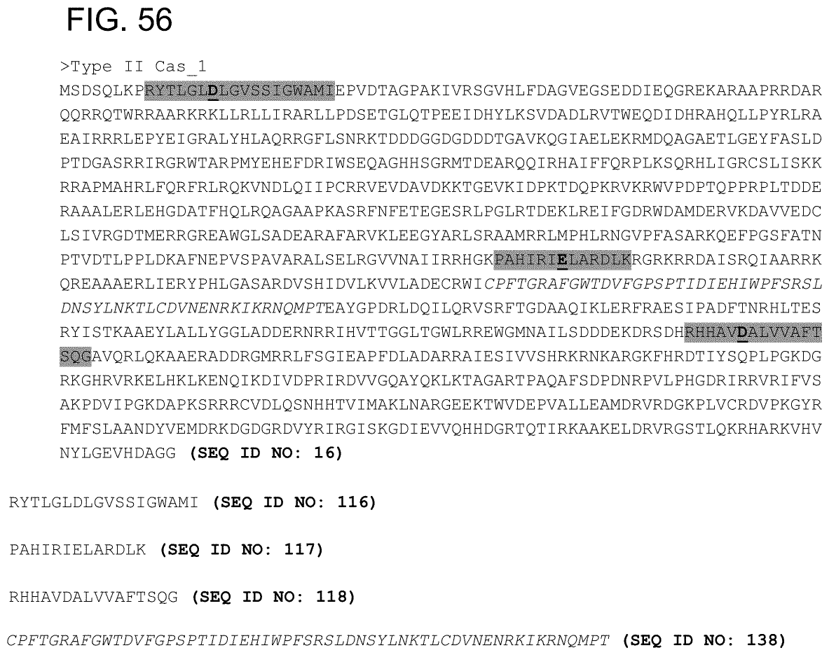

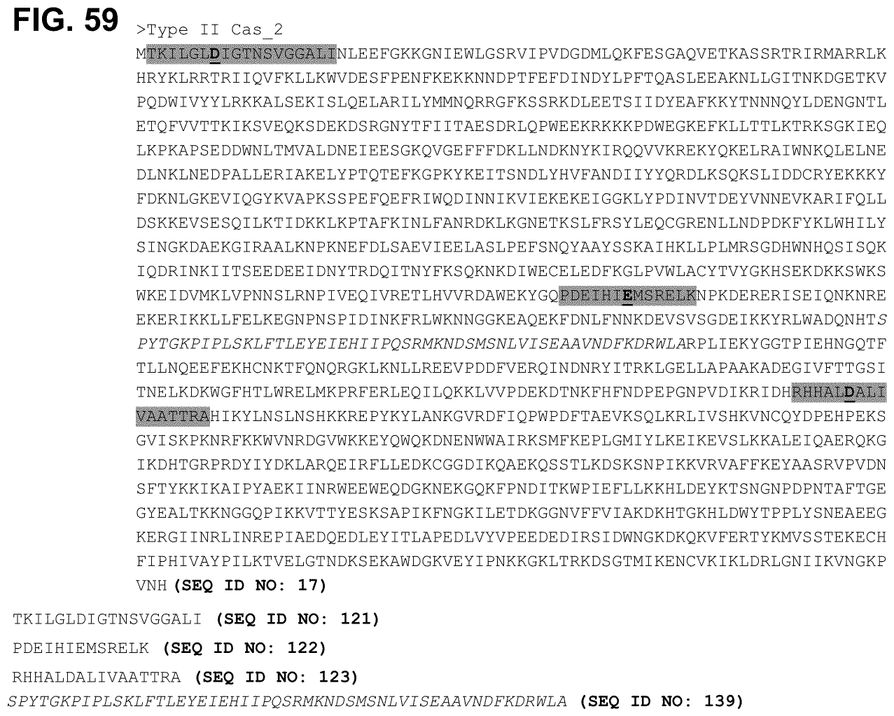

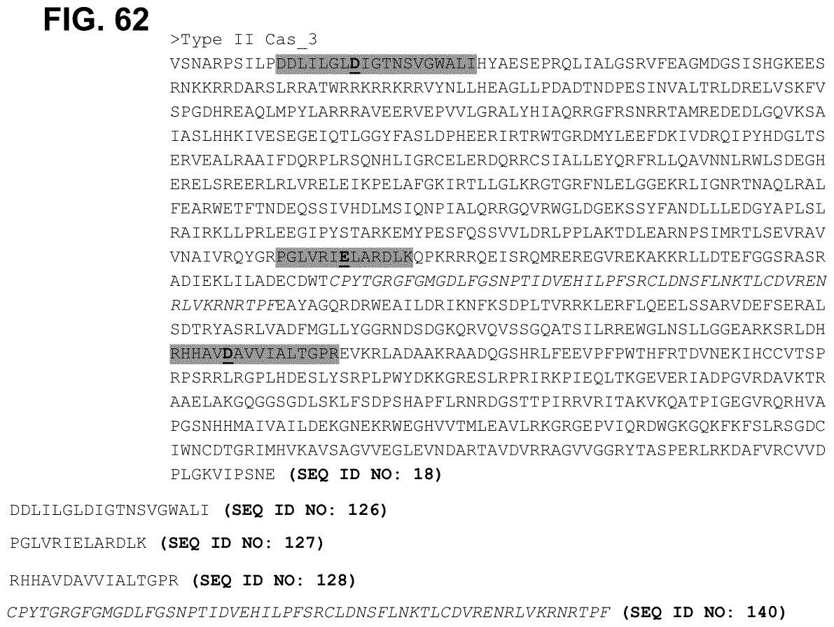

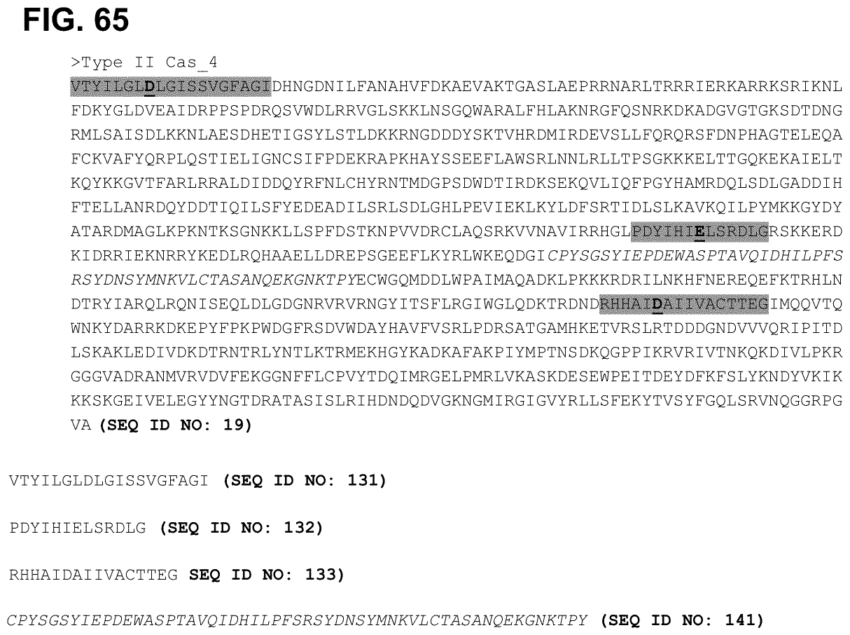

TABLE 7

| SEQ | | | |
|---|---|---|---|
| ID | Exemplary | | |
| NO: | Figure | MOTIF | SEQUENCE |
| 116 | FIG. 56 | RuvC I | RYTLGLDLGVSSIGWAMI |
| 117 | FIG. 56 | RuvC II | PAHIRIELARDLK |
| 118 | FIG. 56 | RuvC III | RHHAVDALVVAFTSQG |
| 121 | FIG. 59 | RuvC I | TKILGLDIGTNSVGGALI |
| 122 | FIG. 59 | RuvC II | PDEIHIEMSRELK |
| 123 | FIG. 59 | RuvC III | RHHALDALIVAATTRA |
| 126 | FIG. 62 | RuvC I | DDLILGLDIGTNSVGWALI |
| 127 | FIG. 62 | RuvC II | PGLVRIELARDLK |
| 128 | FIG. 62 | RuvC III | RHHAVDAVVIALTGPR |
| 131 | FIG. 65 | RuvC I | VTYILGLDLGISSVGFAGI |
| 132 | FIG. 65 | RuvC II | PDYIHIELSRDLG |
| 133 | FIG. 65 | RuvC III | RHHAIDAIIVACTTEG |
| 138 | FIG. 56 | HNH | CPFTGRAFGWTDVFGPSPTIDIEHI |
| | | | WPFSRSLDNSYLNKTLCDVNENRKI |
| | | | KRNQMPT |
| 139 | FIG. 59 | HNH | SPYTGKPIPLSKLFTLEYEIEHIIP |
| | | | QSRMKNDSMSNLVISEAAVNDFKDR |
| | | | WLA |
| 140 | FIG. 62 | HNH | CPYTGRGFGMGDLFGSNPTIDVEHI |
| | | | LPFSRCLDNSFLNKTLCDVRENRLV |
| | | | KRNRTPF |
| 141 | FIG. 65 | HNH | CPYSGSYIEPDEWASPTAVQIDHIL |
| | | | PFSRSYDNSYMNKVLCTASANQEKG |
| | | | NKTPY |

Table 8 shows exemplary amino acid sequences for novel Type II sequences of the disclosure. Genes were identified from metagenomic samples. Scripts were run on the sequences, designed to find CRISPR sequences and accompanying genes encoding proteins showing homology with reported Cas enzymes. Comparative BlastP analyses were performed against sequences deposited in databases (NCBI, LENS), discarding those candidates showing Id % >50 with deposited proteins. Presence of specific domains (e.g. RuvC, HEPN) and catalytic motifs were determined (CD-search, phmmer, UNIPROT).

TABLE 8

| | FIGURE | |
|---|---|---|
| | AND SEQ | |
| NAME | ID | AMINO ACID SEQUENCE |
| Type II Cas_1 | FIG. 56 | MSDSQLKPRYTLGLDLGVSSIGWAMIEPVDTAGPAKIVRSGVHLFDAGVEG |
| | SEQ ID NO: | SEDDIEQGREKARAAPRRDARQQRRQTWRRAARKRKLLRLLIRARLLPDSE |
| | 16 | TGLQTPEEIDHYLKSVDADLRVTWEQDIDHRAHQLLPYRLRAEAIRRRLEP |
| | | YEIGRALYHLAQRRGFLSNRKTDDDGGDGDDDTGAVKQGIAELEKRMDQ |
| | | AGAETLGEYFASLDPTDGASRRIRGRWTARPMYEHEFDRIWSEQAGHHSG |
| | | RMTDEARQQIRHAIFFQRPLKSQRHLIGRCSLISKKRRAPMAHRLFQRFRLR |
| | | QKVNDLQIIPCRRVEVDAVDKKTGEVKIDPKTDQPKRVKRWVPDPTQPPRP |
| | | LTDDERAAALERLEHGDATFHQLRQAGAAPKASRFNFETEGESRLPGLRTD |
| | | EKLREIFGDRWDAMDERVKDAVVEDCLSIVRGDTMERRGREAWGLSADE |
| | | ARAFARVKLEEGYARLSRAAMRRLMPHLRNGVPFASARKQEFPGSFATNP |
| | | TVDTLPPLDKAFNEPVSPAVARALSELRGVVNAIIRRHGKPAHIRIELARDL |
| | | KRGRKRRDAISRQIAARRKQREAAAERLIERYPHLGASARDVSHEDVLKVV |
| | | LADECRWICPFTGRAFGWTDVFGPSPTEDIEHIWPFSRSLDNSYLNKTLCDV |
| | | NENRKIKRNQMPTEAYGPDRLDQILQRVSRFTGDAAQIKLERFRAESIPADF |
| | | TNRHLTESRYISTKAAEYLALLYGGLADDERNRRIHVTTGGLTGWLRREW |
| | | GMNAILSDDDEKDRSDHRHHAVDALVVAFTSQGAVQRLQKAAERADDRG |
| | | MRRLFSGIEAPFDLADARRAIESIVVSHRKRNKARGKFHRDTIYSQPLPGKD |
| | | GRKGHRVRKELHKLKENQIKDIVDPRIRDVVGQAYQKLKTAGARTPAQAF |
| | | SDPDNRPVLPHGDRIRRVRIFVSAKPDVIPGKDAPKSRRRCVDLQSNHHTVI |
| | | MAKLNARGEEKTWVDEPVALLEAMDRVRDGKPLVCRDVPKGYRF1VIFSL |
| | | AANDYVEMDRKDGDGRDVYRIRGISKGDIEVVQHHDGRTQTIRKAAKELD |
| | | RVRGSTLQKRHARKVHVNYLGEVHDAGG |
| Type II Cas_2 | FIG. 59 | MTKILGLDIGTNSVGGALINLEEFGKKGNIEWLGSRVIPVDGDMLQKFESG |
| | SEQ ID NO: | AQVETKASSRTRIRMARRLKHRYKLRRTRIIQVFKLLKWVDESFPENFKEK |
| | 17 | KNNDPTFEFDINDYLPFTQASLEEAKNLLGITNKDGETKVPQDWIVYYLRK |
| | | KALSEKISLQELARILYMMNQRRGFKSSRKDLEETSIIDYEAFKKYTNNNQY |
| | | LDENGNTLETQFVVTTKEKSVEQKSDEKDSRGNYTFIITAESDRLQPWEEKR |
| | | KKKPDWEGKEFKLLTTLKTRKSGKIEQLKPKAPSEDDWNLTMVALDNEIE |
| | | ESGKQVGEFFFDKLLNDKNYKIRQQVVKREKYQKELRAIWNKQLELNEDL |
| | | NKLNEDPALLERIAKELYPTQTEFKGPKYKEITSNDLYHVFANDIIYYQRDL |
| | | KSQKSLIDDCRYEKKKYFDKNLKEVIQGYKVAPKSSPEFQEFRIWQDINNI |
| | | KVIEKEKEIGGKLYPDINVTDEYVNNEVKARIFQLLDSKKEVSESQILKTIDK |
| | | KLKPTAFKINLFANRDKLKGNETKSLFRSYLEQCGRENLLNDPDKFYKLW |
| | | HILYSINGKDAEKGIRAALKNPKNEFDLSAEVIEELASLPEFSNQYAAYSSK |
| | | AIHKLLPLMRSGDHWNHQSISQKIQDRINKIITSEEDEEIDNYTRDQITNYFK |
| | | SQKNKDIWECELEDFKGLPVWLACYTVYGKHSEKDKKSWKSWKEEDVMK |
| | | LVPNNSLRNPIVEQIVRETLHVVRDAWEKYGQPDEIHIEMSRELKNPKDERE |
| | | RISEIQNKNREEKERIKKLLFELKEGNPNSPIDINKFRLWKNNGGKEAQEKF |
| | | DNLFNNKDEVSVSGDEEKKYRLWADQNHTSPYTGKPIPLSKLFTLEYEIEHII |
| | | PQSRKNDSMSNLVISEAAVNDFKDRWLARPLIEKYGGTPIEHNGQTFTLL |
| | | NQEEFEKHCNKTFQNQRGKLKNLLREEVPDDFVERQINDNRYITRKLGELL |
| | | APAAKADEGIVFTTGSITNELKDKWGFHTLWRELMKPRFERLEQILQKKLV |
| | | VPDEKDTNKFHFNDPEPGNPVDIKRIDHRHHALDALIVAATTRAHIKYLNSL |
| | | NSHKKREPYKYLANKGVRDFIQPWPDFTAEVKSQLKRLIVSHKVNCQYDP |
| | | EHPEKSGVISKPKNRFKKWVNRDGVWKKEYQWQKDNENWWAERKSMFK |
| | | EPLGMIYLKEIKEVSLKKALEIQAERQKGIKDHTGRPRDYIYDKLARQEIRF |
| | | LLEDKCGGDEKQAEKQSSTLKDSKSNPIKKVRVAFFKEYAASRVPVDNSFT |
| | | YKKIKAIPYAEKIINRWEEWEQDGKNEKGQKFPNDITKWPIEFLLKKHLDE |
| | | YKTSNGNPDPNTAFTGEGYEALTKKNGGQPIKKVTTYESKSAPIKFNGKILE |
| | | TDKGGNVFFVIAKDKHTGKHLDWYTPPLYSNEAEEGKERGIINRLINREPIA |
| | | EDQEDLEYITLAPEDLVYVPEEDEDIRSEDWNGKDKQKVFERTYKMVSSTE |
| | | KECIEFIPHIVAYPILKTVELGTNDKSEKAWDGKVEYIPNKKGKLTRKDSGT |
| | | MEKENCVKIKLDRLGNIIKVNGKPVNH |
| Type II Cas_3 | FIG. 62 | VSNARPSILPDDLILGLDIGTNSVGWALIHYAESEPRQLIALGSRVFEAGMD |
| | SEQ ID NO: | GSISHGKEESRNKKRRDARSLRRATWRRKRRKRRVYNLLHEAGLLPDADT |
| | 18 | NDPESINVALTRLDRELVSKEVSPGDHREAQL1VEPYLARRRAVEERVEPVVL |
| | | GRALYHIAQRRGFRSNRRTAMREDEDLGQVKSAIASLHHKIVESEGEIQTL |
| | | GGYFASLDPHEERIRTRWTGRDMYLEEFDKIVDRQIPYHDGLTSERVEALR |
| | | AAIFDQRPLRSQNHLIGRCELERDQRRCSIALLEYQRFRLLQAVNNLRWLS |
| | | DEGHERELSREERLRLVRELEIKPELAFGKIRTLLGLKRGTGRFNLELGGEK |
| | | RLIGNRTNAQLRALFEARWETFTNDEQSSIVHDLMSIQNPIALQRRGQVRW |
| | | GLDGEKSSYFANDLLLEDGYAPLSLRAIRKLLPRLEEGIPYSTARKEMYPES |
| | | FQSSVVLDRLPPLAKTDLEARNPSEMRTLSEVRAVVNAIVRQYGRPGLVRIE |
| | | LARDLKQPKRRRQEISRQMREREGVREKAKKRLLDTEFGGSRASRADIEKL |
| | | ILADECDWTCPYTGRGEGMGDLEGSNPTIDVEHILPFSRCLDNSFLNKTLCD |
| | | VRENRLVKRNRTPFEAYAGQRDRWEAILDRIKNFKSDPLTVRRKLERFLQE |
| | | ELSSARVDEFSERALSDTRYASRLVADFMGLLYGGRNDSDGKQRVQVSSG |
| | | QATSILRREWGLNSLLGGEARKSRLDHRHHAVDAVVIALTGPREVKRLAD |
| | | AAKRAADQGSHRLFEEVPFPWTHERTDVNEKIHCCVTSPRPSRRLRGPLHD |
| | | ESLYSRPLPWYDKKGRESLRPRIRKPIEQLTKGEVERIADPGVRDAVKTRAA |
| | | ELAKGQGGSGDLSKLFSDPSHAPFLRNRDGSTTPIRRVRITAKVKQATPIGE |
| | | GVRQRHVAPGSNHHMAIVAILDEKGNEKRWEGHVVTMLEAVLRKGRGEP |
| | | VIQRDWGKGQIUKFSLRSGDCIWNCDTGREMHVKAVSAGVVEGLEVNDA |
| | | RTAVDVRRAGVVGGRYTASPERLRKDAFVRCVVDPLGKVIPSNE |
| Type II Cas_4 | FIG. 65 | VTYILGLDLGISSVGFAGEDHNGDNILFANAHVFDKAEVAKTGASLAEPRR |
| | SEQ ID NO: | NARLTRRRIERKARRKSRIKNLFDKYGLDVEAIDRPPSPDRQSVWDLRRVG |
| | 19 | LSKKLNSGQWARALFHLAKNRGFQSNRKDKADGVGTGKSDTDNGRMLSA |
| | | ISDLKKNLAESDHETIGSYLSTLDKKRNGDDDYSKTVHRDMIRDEVSLLFQ |
| | | RQRSEDNPHAGTELEQAFCKVAFYQRPLQSTIELIGNCSIFPDEKRAPKHAY |
| | | SSEEFLAWSRLNNLRLLTPSGKKKELTTGQKEKAIELTKQYKKGVTFARLR |
| | | RALDIDDQYRFNLCHYRNTMDGPSDWDTIRDKSEKQVLIQFPGYHAMRDQ |
| | | LSDLGADDHIFTELLANRDQYDDTIQILSFYEDEADILSRLSDLGHLPEVIEK |
| | | LKYLDFSRTIDLSLKAVKQILPYMKKGYDYATARDMAGLKPKNTKSGNKK |
| | | LLSPEDSTKNPVVDRCLAQSRKVVNAVIRRHGLPDYIHIELSRDLGRSKKER |
| | | DKIDRRIEKNRRYKEDLRQHAAELLDREPSGEEFLKYRLWKEQDGICPYSG |
| | | SYIEPDEWASPTAVQIDHILPFSRSYDNSYMNKVLCTASANQEKGNKTPYE |
| | | CWGQMDDLWPAIMAQADKLPKKKRDRILNKHFNEREQEFKTRHLNDTRY |
| | | IARQLRQNISEQLDLGDGNRVRVRNGYITSFLRGIWGLQDKTRDNDRHHAI |
| | | DAIIVACTTEGEMQQVTQWNKYDARRKDKEPYFPKPWDGERSDVWDAYH |
| | | AVEVSRLPDRSATGAMEEKETVRSLRTDDDGNDVVVQRIPITDLSKAKLEDI |
| | | VDKDTRNTRLYNTLKTRMEKHGYKADKAFAKPIYMPTNSDKQGPPIKRVR |
| | | IVTNKQKDIVLPKRGGGVADRANMVRVDVFEKGGNEFLCPVYTDQUVIRGE |
| | | LPMRLVKASKDESEWPEITDEYDFKFSLYKNDYVKIKKKSKGEIVELEGYY |
| | | NGTDRATASISLRIEEDNDQDVGKNGMIRGIGVYRLLSFEKYTVSYFGQLSR |
| | | VNQGGRPGVA |

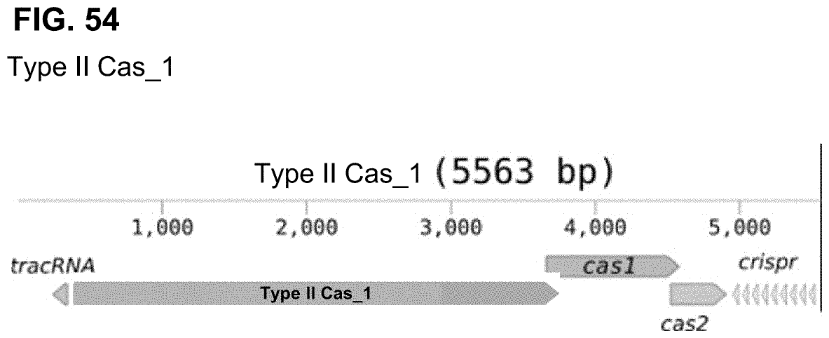

SEQ ID NO: 16 represents a novel Type II variant of the disclosure, Type II Cas_1, (1091 amino acids in length FIG. 54 is a schematic representation of the organization of the CRISPR Cas cluster loci around the Type II Cas_1 gene of the disclosure. FIG. 56 shows the amino acid sequence of Type II Cas_1 (SEQ ID NO: 16) with the RuvC motifs underlined/highlighted. The RuvC I, II and III motifs are sequentially shown (highlighted in gray). The HNH domain is shown in italics. The Campylovacter_jeju Type II sequence referenced in Shmakov et al., 2015 was used as a reference for identification of the Ruv motifs.

In some embodiments the Type II CRISPR-Cas RNA-guided endonuclease of the disclosure comprises the amino acid sequence of SEQ ID NO: 16 and proteins with at least 30%-99.5% sequence identity thereto. Accordingly, provided herein are proteins comprising the amino acid sequence of SEQ ID NO: 16 and proteins with at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto. Also provided herein are nucleic acids encoding the proteins comprising the amino acid sequence of SEQ ID NO: 16 and proteins with at least 30%-99.5% sequence identity thereto.

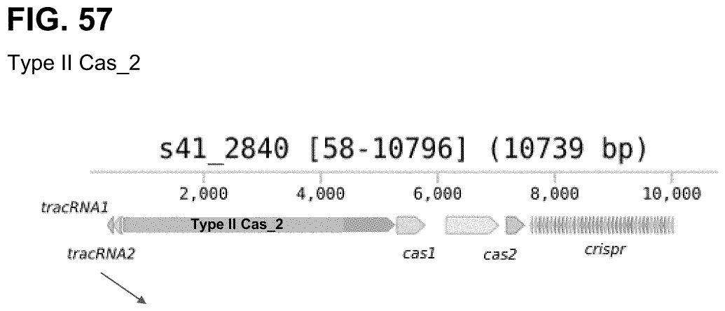

SEQ ID NO: 17 represents a novel Type II variant of the disclosure, Type II Cas_2, (1565 amino acids in length). FIG. 57 is a schematic representation of the organization of the CRISPR Cas cluster loci around the Type II Cas_2 gene of the disclosure. There are two putative tracRNA (tracRNA1, tracRNA2). Likely only one has sufficient complementarity to enable stable interaction. FIG. 59 shows the amino acid sequence of Type II Cas_2 (SEQ ID NO: 17) with the RuvC motifs underlined/highlighted. The RuvC I, II and III motifs are sequentially shown (highlighted in gray). The HNH domain is shown in italics. The Campylovacter_jeju Type II sequence referenced in Shmakov et al., 2015 was used as a reference for identification of the Ruv motifs.

In some embodiments the Type II CRISPR-Cas RNA-guided endonuclease of the disclosure comprises the amino acid sequence of SEQ ID NO: 17 and proteins with at least 30%-99.5% sequence identity thereto. Accordingly, provided herein are proteins comprising the amino acid sequence of SEQ ID NO: 17 and proteins with at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto. Also provided herein are nucleic acids encoding the proteins comprising the amino acid sequence of SEQ ID NO: 17 and proteins with at least 30%-99.5% sequence identity thereto.

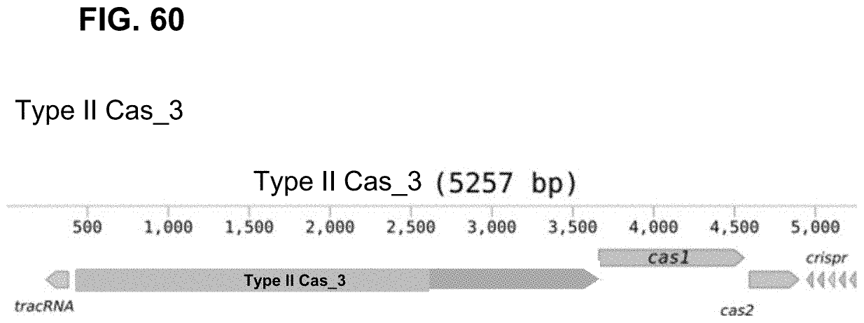

SEQ ID NO: 18 represents a novel Type II variant of the disclosure, Type II Cas_3, (1064 amino acids in length). FIG. 60 is a schematic representation of the organization of the CRISPR Cas cluster loci around the Type II Cas_3 gene of the disclosure. FIG. 62 shows the amino acid sequence of Type II Cas_3 (SEQ ID NO: 18) with the RuvC motifs underlined/highlighted. The RuvC I, II and III motifs are sequentially shown (highlighted in gray). The HNH domain is shown in italics. The Campylovacter_jeju Type II sequence referenced in Shmakov et al., 2015 was used as a reference for identification of the Ruv motifs.

In some embodiments the Type II CRISPR-Cas RNA-guided endonuclease of the disclosure comprises the amino acid sequence of SEQ ID NO: 18 and proteins with at least 30%-99.5% sequence identity thereto. Accordingly, provided herein are proteins comprising the amino acid sequence of SEQ ID NO: 18 and proteins with at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto. Also provided herein are nucleic acids encoding the proteins comprising the amino acid sequence of SEQ ID NO: 18 and proteins with at least 30%-99.5% sequence identity thereto.

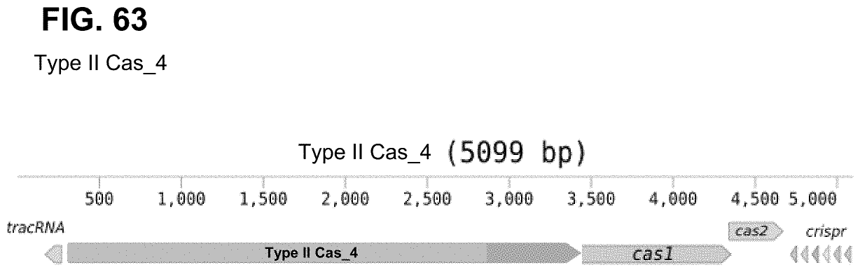

SEQ ID NO: 19 represents a novel Type II variant of the disclosure, Type II Cas_4, (1024 amino acids in length). FIG. 63 is a schematic representation of the organization of the CRISPR Cas cluster loci around the Type II Cas_4 gene of the disclosure. FIG. 65 shows the amino acid sequence of Type II Cas_4 (SEQ ID NO: 19) with the RuvC motifs underlined/highlighted. The RuvC I, II and III motifs are sequentially shown (highlighted in gray). The HNH domain is shown in italics. The Campylovacter_jeju Type II sequence referenced in Shmakov et al., 2015 was used as a reference for identification of the Ruv motifs.

In some embodiments the Type II CRISPR-Cas RNA-guided endonuclease of the disclosure comprises the amino acid sequence of SEQ ID NO: 19 and proteins with at least 30%-99.5% sequence identity thereto. Accordingly, provided herein are proteins comprising the amino acid sequence of SEQ ID NO: 19 and proteins with at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto. Also provided herein are nucleic acids encoding the proteins comprising the amino acid sequence of SEQ ID NO: 19 and proteins with at least 30%-99.5% sequence identity thereto.

Table 9 provides exemplary nucleic acid sequences for encoding certain Type II sequences of the disclosure. Also provided are exemplary E. coli codon optimized nucleic acid sequences for encoding certain Type II sequences of the disclosure.

Accordingly, provided herein are exemplary nucleic acid sequences encoding the Type II CRISPR-Cas RNA-guided endonucleases of the disclosure. In some embodiments, a Type II CRISPR-Cas RNA-guided endonuclease is encoded by a nucleic acid sequence comprising or consisting of the sequence of any one of SEQ ID NOs: 51-58, or a nucleic acid sequence with at least 30%, at least 35%, at least 40%, at least 45%, at least 50%, at least 55%, at least 60%, at least 65%, at least 70%, at least 75%, at least 80%, at least 85%, at least 90%, at least 95%, or at least 99.5% sequence identity thereto.

TABLE 9

| | | CODON OPTIMIZED NUCLEIC ACID |
|---|---|---|
| NAME | NUCLEIC ACID SEQUENCE | SEQUENCE |
| Type II | ATGTCAGATTCTCAACTGAAACCACGTTACACCCT | ATGAGCGACAGCCAACTGAAGCCGCGTTACACCCTGGG |
| CaS_1 | CGGTCTGGACCTCGGCGTTTCATCGATCGGCTGGG | CCTGGATCTGGGTGTGAGCAGCATCGGCTGGGCGATGA |
| | CCATGATCGAGCCGGTTGACACAGCGGGACCGGCC | TTGAACCGGTTGACACCGCGGGTCCGGCGAAGATTGTT |
| | AAAATCGTCCGCAGCGGGGTCCATCTGTTTGATGC | CGTAGCGGCGTTCACCTGTTCGATGCGGGTGTGGAAGG |
| | GGGCGTCGAGGGCAGCGAAGACGATATCGAGCAA | CAGCGAGGACGATATTGAACAGGGTCGTGAGAAGGCG |
| | GGCCGCGAGAAAGCGCGTGCCGCTCCACGCCGCGA | CGTGCGGCGCCGCGTCGTGATGCGCGTCAGCAGCGTCG |
| | CGCCCGCCAGCAGCGTCGGCAGACCTGGCGGCGGG | TCAGACCTGGCGTCGTGCGGCGCGTAAGCGTAAACTGC |
| | CCGCACGGAAACGAAAGCTGCTGCGTCTTCTGATC | TGCGTCTGCTGATCCGTGCGCGTCTGCTGCCGGACAGC |
| | CGCGCTCGCCTGCTGCCGGATTCGGAAACCGGCCT | GAAACCGGTCTGCAAACCCCGGAGGAAATTGATCACTA |
| | GCAAACGCCGGAGGAAATCGATCATTACCTCAAAT | CCTGAAGAGCGTGGATGCGGACCTGCGTGTTACCTGGG |
| | CCGTTGACGCCGACCTACGCGTCACCTGGGAACAG | AGCAAGATATCGACCACCGTGCGCACCAGCTGCTGCCG |
| | GACATTGATCATCGCGCCCACCAGTTGCTGCCCTA | TATCGTCTGCGTGCGGAAGCGATCCGTCGTCGTCTGGA |
| | CCGCCTGCGCGCCGAAGCGATCCGGCGAAGGCTCG | ACCGTACGAGATTGGTCGTGCGCTGTATCACCTGGCGC |
| | AGCCGTACGAGATCGGCCGCGCCTTGTACCACCTC | AGCGTCGTGGCTTTCTGAGCAACCGTAAAACCGACGAT |
| | GCCCAGCGGCGCGGATTTCTGAGCAACCGCAAGAC | GACGGTGGCGACGGCGATGACGATACCGGTGCGGTGA |
| | TGACGACGACGGCGGCGATGGCGACGACGACACG | AGCAAGGCATCGCGGAGCTGGAAAAACGTATGGATCA |
| | GGCGCCGTCAAGCAAGGCATCGCCGAGTTGGAAA | GGCGGGTGCGGAAACCCTGGGCGAGTACTTTGCGAGCC |
| | AGCGGATGGACCAAGCCGGCGCGGAGACGCTCGG | TGGATCCGACCGATGGTGCGAGCCGTCGTATTCGTGGC |
| | CGAATACTTCGCCTCGCTTGATCCCACCGACGGCG | CGTTGGACCGCGCGTCCGATGTATGAGCACGAATTTGA |
| | CGTCCCGGCGCATCCGGGGCCGCTGGACCGCGCGT | CCGTATTTGGAGCGAGCAGGCGGGTCACCACAGCGGTC |
| | CCGATGTACGAGCATGAGTTCGACCGCATCTGGTC | GTATGACCGATGAAGCGCGTCAGCAAATCCGTCACGCG |
| | GGAGCAGGCCGGCCACCACTCGGGCCGCATGACCG | ATTTTCTTTCAGCGTCCGCTGAAGAGCCAACGTCACCTG |
| | ACGAGGCGCGTCAGCAGATCCGCCACGCCATCTTT | ATCGGCCGTTGCAGCCTGATTAGCAAGAAACGTCGTGC |
| | TTTCAGCGACCACTCAAAAGTCAGCGTCACCTGAT | GCCGATGGCGCACCGTCTGTTCCAGCGTTTTCGTCTGCG |
| | CGGCCGTTGCTCTTTGATTTCTAAAAAACGGCGCG | TCAAAAAGTTAACGACCTGCAGATCATTCCGTGCCGTC |
| | CCCCCATGGCCCATCGTCTGTTCCAGCGATTCCGCC | GTGTTGAGGTGGATGCGGTGGACAAGAAAACCGGTGA |
| | TGCGGCAAAAGGTCAACGACCTGCAGATCATCCCG | AGTTAAGATCGACCCGAAAACCGATCAACCGAAGCGT |
| | TGCAGGCGCGTCGAGGTCGACGCCGTTGACAAGAA | GTGAAACGTTGGGTTCCGGACCCGACCCAGCCGCCGCG |
| | GACCGGCGAAGTCAAAATCGACCCCAAAACCGAC | TCCGCTGACCGATGATGAGCGTGCTGCGGCGCTGGAAC |
| | CAGCCCAAACGCGTCAAGCGCTGGGTCCCCGATCC | GTCTGGAGCACGGTGATGCGACCTTTCATCAGCTGCGT |
| | CACCCAGCCGCCTCGCCCGTTGACCGACGACGAGC | CAAGCGGGTGCGGCGCCGAAGGCGAGCCGTTTCAACTT |
| | GGGCCGCGGCGCTCGAGCGCCTCGAACATGGCGAC | TGAGACCGAAGGTGAAAGCCGTCTGCCGGGTCTGCGTA |
| | GCGACTTTTCATCAGCTCCGTCAGGCGGGAGCCGC | CCGACGAAAAGCTGCGTGAGATCTTTGGCGATCGTTGG |
| | GCCAAAGGCCTCACGCTTTAACTTCGAGACCGAGG | GACGCGATGGATGAGCGTGTGAAAGACGCGGTGGTTG |
| | GCGAGTCACGGCTTCCGGGTCTGCGAACCGATGAA | AAGATTGCCTGAGCATTGTTCGTGGTGACACCATGGAG |
| | AAGCTGAGAGAAATATTCGGCGACCGCTGGGACGC | CGTCGTGGTCGTGAGGCGTGGGGCCTGAGCGCGGATGA |
| | GATGGATGAGCGAGTAAAAGACGCCGTCGTCGAG | GGCGCGTGCGTTCGCGCGTGTTAAACTGGAGGAAGGTT |
| | GACTGTCTTTCGATCGTCCGGGGCGACACGATGGA | ATGCGCGTCTGAGCCGTGCGGCGATGCGTCGTCTGATG |
| | GAGGCGAGGCCGCGAGGCGTGGGGGTTGTCGGCC | CCGCACCTGCGTAACGGTGTGCCGTTTGCGAGCGCGCG |
| | GACGAGGCCCGCGCCTTCGCCCGTGTCAAGCTGGA | TAAGCAGGAATTCCCGGGCAGCTTTGCGACCAACCCGA |
| | GGAAGGCTACGCCCGGCTGTCCCGCGCGGCGATGC | CCGTTGACACCCTGCCGCCGCTGGATAAAGCGTTTAAC |
| | GGCGGCTGATGCCTCACCTGCGGAACGGCGTCCCG | GAGCCGGTTAGCCCGGCGGTTGCGCGTGCGCTGAGCGA |
| | TTCGCATCGGCACGCAAACAGGAATTTCCCGGATC | ACTGCGTGGTGTGGTTAACGCGATCATTCGTCGTCACG |
| | CTTCGCGACCAACCCCACCGTCGACACCCTCCCGC | GCAAGCCGGCGCACATCCGTATTGAGCTGGCGCGTGAC |
| | CACTGGACAAGGCGTTCAATGAGCCGGTCAGTCCC | CTGAAGCGTGGCCGTAAACGTCGTGATGCGATCAGCCG |
| | GCGGTCGCGCGGGCGCTGTCGGAGCTGCGCGGCGT | TCAAATTGCGGCGCGTCGTAAGCAGCGTGAAGCTGCGG |
| | GGTGAATGCGATCATCCGCCGCCACGGCAAGCCCG | CGGAGCGTCTGATCGAACGTTATCCGCACCTGGGTGCG |
| | CCCATATCCGGATCGAGCTCGCCCGCGACCTGAAG | AGCGCGCGTGATGTGAGCCACATCGATGTTCTGAAAGT |
| | CGTGGCCGCAAACGCCGCGACGCCATCAGTCGACA | GGTTCTGGCGGACGAGTGCCGTTGGATTTGCCCGTTCA |
| | GATCGCCGCCCGGCGAAAGCAGCGGGAGGCCGCG | CCGGCCGTGCGTTTGGTTGGACCGACGTGTTCGGTCCG |
| | GCCGAACGGCTCATCGAGCGTTACCCCCACCTCGG | AGCCCGACCATCGATATTGAACACATTTGGCCGTTTAG |
| | CGCGTCGGCCCGCGACGTCTCCCATATCGACGTGC | CCGTAGCCTGGACAACAGCTACCTGAACAAAACCCTGT |
| | TCAAAGTCGTCCTCGCCGACGAGTGCCGCTGGATC | GCGATGTGAACGAGAACCGTAAGATCAAACGTAACCA |
| | TGTCCGTTTACCGGACGGGCGTTCGGCTGGACCGA | AATGCCGACCGAAGCGTATGGTCCGGACCGTCTGGATC |
| | TGTCTTCGGCCCCAGCCCGACGATCGACATCGAGC | AGATTCTGCAACGTGTTAGCCGTTTCACCGGTGATGCG |
| | ACATCTGGCCATTCAGCCGATCGCTCGACAATTCCT | GCGCAGATCAAGCTGGAGCGTTTCCGTGCGGAAAGCAT |
| | ATCTCAACAAAACGCTCTGCGACGTGAACGAGAAC | TCCGGCGGATTTTACCAACCGTCACCTGACCGAGAGCC |
| | CGCAAAATCAAGCGAAACCAGATGCCCACCGAAG | GTTACATCAGCACCAAAGCGGCGGAATACCTGGCGCTG |
| | CCTACGGCCCCGACCGGCTCGACCAGATCCTCCAG | CTGTATGGTGGCCTGGCGGACGATGAGCGTAACCGTCG |
| | CGCGTCTCCCGCTTCACCGGCGACGCCGCACAGAT | TATCCACGTTACCACCGGTGGCCTGACCGGTTGGCTGC |
| | CAAGCTGGAACGCTTCCGCGCCGAGTCGATCCCCG | GTCGTGAGTGGGGCATGAACGCGATTCTGAGCGACGAT |
| | CCGATTTCACCAATCGGCATCTCACCGAGTCCCGCT | GACGAAAAGGACCGTAGCGATCACCGTCATCATGCGGT |
| | ACATCTCGACCAAGGCCGCCGAATATCTCGCCCTG | GGATGCGCTGGTTGTGGCGTTCACCAGCCAGGGTGCGG |
| | CTTTACGGCGGGCTTGCAGACGACGAGCGCAATCG | TTCAGCGTCTGCAAAAAGCGGCGGAACGTGCGGATGAC |
| | CCGCATTCACGTGACCACGGGCGGGTTGACCGGCT | CGTGGTATGCGTCGTCTGTTCAGCGGTATTGAAGCGCC |
| | GGCTGCGTCGGGAATGGGGGATGAACGCCATCCTC | GTTTGACCTGGCGGATGCGCGTCGTGCGATCGAAAGCA |
| | TCCGACGATGATGAGAAAGACCGAAGCGACCATC | TTGTGGTTAGCCACCGTAAGCGTAACAAAGCGCGTGGC |
| | GCCACCACGCCGTGGACGCCCTGGTGGTCGCCTTC | AAGTTTCACCGTGACACCATTTACAGCCAACCGCTGCC |
| | ACGTCCCAGGGCGCGGTCCAGCGGTTGCAGAAGGC | GGGCAAGGATGGCCGTAAAGGTCACCGTGTGCGTAAG |
| | GGCCGAGCGGGCCGACGACCGGGGCATGCGCCGG | GAGCTGCACAAGCTGAAAGAAAACCAGATCAAAGACA |
| | CTTTTCTCCGGCATCGAAGCGCCGTTTGATCTCGCC | TTGTTGATCCGCGTATCCGTGACGTGGTTGGTCAGGCGT |
| | GACGCACGTCGCGCGATCGAGAGCATCGTCGTCAG | ATCAAAAGCTGAAAACCGCGGGTGCGCGTACCCCGGC |
| | CCACCGAAAACGAAACAAGGCCCGCGGCAAGTTC | GCAAGCGTTCAGCGATCCGGACAACCGTCCGGTGCTGC |
| | CATAGAGATACGATCTACAGCCAGCCCCTGCCCGG | CGCATGGTGACCGTATCCGTCGTGTGCGTATTTTTGTTA |
| | CAAGGACGGCAGGAAGGGCCACCGCGTCCGCAAG | GCGCGAAACCGGACGTTATCCCGGGCAAGGATGCGCC |
| | GAACTGCACAAACTCAAGGAAAACCAGATCAAGG | GAAAAGCCGTCGTCGTTGCGTGGATCTGCAGAGCAACC |
| | ACATCGTCGACCCCCGCATCCGCGACGTGGTCGGC | ACCACACCGTTATTATGGCGAAGCTGAACGCGCGTGGT |
| | CAGGCGTATCAGAAGCTGAAAACCGCCGGCGCGA | GAGGAAAAAACCTGGGTGGATGAGCCGGTTGCGCTGCT |
| | GGACCCCGGCCCAGGCCTTCAGTGACCCGGACAAC | GGAAGCGATGGACCGTGTGCGTGATGGCAAGCCGCTG |
| | CGCCCCGTCCTGCCCCACGGCGACCGCATCCGCCG | GTGTGCCGTGATGTTCCGAAAGGCTACCGTTTCATGTTT |
| | CGTCCGCATCTTCGTCAGCGCCAAGCCGGACGTGA | AGCCTGGCGGCGAACGACTATGTGGAGATGGATCGTAA |
| | TCCCCGGCAAAGACGCGCCCAAATCACGCCGTCGC | GGATGGTGACGGCCGTGACGTTTACCGTATCCGTGGCA |
| | TGCGTCGATCTACAGTCCAATCACCACACGGTGAT | TTAGCAAAGGTGACATCGAGGTGGTTCAACACCACGAT |
| | CATGGCCAAACTGAACGCCCGCGGCGAGGAAAAG | GGTCGTACCCAGACCATTCGCAAAGCGGCGAAAGAACT |
| | ACATGGGTCGATGAACCGGTCGCCTTGCTGGAGGC | GGACCGTGTGCGTGGCAGCACCCTGCAGAAGCGTCACG |
| | GATGGACCGGGTCCGCGACGGCAAGCCTCTGGTCT | CGCGTAAAGTGCACGTTAACTATCTGGGTGAAGTTCAC |
| | GTCGCGACGTGCCGAAGGGATACAGGTTTATGTTT | GATGCGGGTGGCTAG (SEQ ID NO: 52) |
| | TCGCTGGCGGCAAATGACTACGTGGAAATGGATCG | |
| | TAAAGATGGTGATGGCCGCGATGTCTACCGAATCC | |
| | GAGGCATCTCGAAAGGAGACATTGAAGTCGTGCAG | |
| | CACCATGACGGCAGGACACAAACGATCCGCAAGG | |
| | CCGCCAAGGAACTGGATCGAGTCCGCGGATCGACA | |
| | CTTCAGAAACGTCACGCCCGAAAGGTGCACGTGAA | |
| | CTATCTCGGGGAGGTGCACGATGCCGGCGGCTGA | |
| | (SEQ ID NO: 51) | |
| Type II | ATGACTAAAATTTTAGGACTCGACATTGGTACAAA | ATGACCAAGATCCTGGGTCTGGACATTGGCACCAACAG |
| Cas_2 | TTCAGTGGGTGGCGCACTGATTAATTTGGAAGAAT | CGTGGGTGGCGCGCTGATCAACCTGGAGGAATTCGGTA |
| | TCGGTAAAAAAGGCAATATAGAATGGCTTGGTAGT | AGAAAGGCAACATCGAGTGGCTGGGTAGCCGTGTGATT |
| | AGGGTAATTCCAGTAGATGGCGATATGCTTCAAAA | CCGGTTGACGGCGATATGCTGCAGAAGTTTGAGAGCGG |
| | ATTTGAAAGTGGGGCCCAGGTGGAAACCAAAGCTT | TGCGCAAGTGGAAACCAAAGCGAGCAGCCGTACCCGT |
| | CCTCAAGAACACGAATAAGGATGGCAAGAAGATT | ATCCGTATGGCGCGTCGTCTGAAGCACCGTTACAAACT |
| | AAAACATCGTTATAAACTTAGAAGAACACGCATAA | GCGTCGTACCCGTATCATTCAGGTGTTCAAACTGCTGA |
| | TTCAAGTGTTCAAATTACTTAAATGGGTTGACGAA | AGTGGGTTGATGAGAGCTTTCCGGAAAACTTCAAGGAG |
| | AGTTTCCCCGAAAACTTCAAAGAAAAAAAGAATAA | AAGAAAAACAACGACCCGACCTTTGAGTTCGACATCAA |
| | CGATCCAACATTTGAATTTGATATTAATGACTATCT | CGATTATCTGCCGTTTACCCAAGCGAGCCTGGAGGAAG |
| | CCCCTTCACTCAAGCATCCCTTGAAGAGGCAAAGA | CGAAAAACCTGCTGGGTATCACCAACAAGGATGGCGA |
| | ACTTATTAGGAATTACCAACAAAGATGGAGAAACC | AACCAAAGTGCCGCAGGACTGGATTGTTTACTATCTGC |
| | AAAGTACCACAGGATTGGATTGTTTATTATTTGAG | GTAAGAAAGCGCTGAGCGAGAAGATCAGCCTGCAGGA |
| | GAAAAAAGCGCTTTCCGAGAAAATCTCACTTCAGG | ACTGGCGCTATTCTGTACATGATGAACCAACGTCGTG |
| | AGCTTGCCCGTATACTCTATATGATGAATCAAAGA | GTTTCAAGAGCAGCCGTAAAGATCTGGAGGAAACCAG |
| | AGGGGGTTTAAAAGTAGTAGAAAAGACTTGGAGG | CATCATTGACTACGAGGCGTTTAAGAAATATACCAACA |
| | AAACTTCTATTATAGATTATGAAGCATTTAAAAAA | ACAACCAGTACCTGGACGAGAACGGTAACACCCTGGA |
| | TATACGAATAATAACCAATATTTGGATGAAAATGG | AACCCAATTCGTGGTTACCACCAAGATCAAAAGCGTGG |
| | CAATACACTTGAGACACAATTTGTTGTTACTACGA | AGCAGAAGAGCGACGAAAAAGATAGCCGTGGCAACTA |
| | AAATTAAATCAGTAGAGCAGAAGAGTGATGAGAA | CACCTTTATCATTACCGCGGAAAGCATCGTCTGCAGC |
| | AGATAGTAGAGGAAATTATACATTTATCATTACAG | CGTGGGAGGAAAAACGTAAGAAAAAGCCGGACTGGGA |
| | CCGAAAGTGATAGATTACAACCTTGGGAGGAAAA | GGGTAAAGAGTTCAAGCTGCTGACCACCCTGAAAACCC |
| | GAGAAAGAAAAAACCTGATTGGGAAGGAAAGGAG | GTAAGAGCGGCAAAATCGAACAACTGAAGCCGAAAGC |
| | TTTAAACTTTTAACAACTCTTAAAACAAGAAAAAG | GCCGAGCGAGGACGATTGGAACCTGACCATGGTGGCG |
| | TGGTAAAATTGAACAATTAAAGCCAAAGGCTCCTT | CTGGACAACGAAATTGAGGAAAGCGGCAAGCAGGTTG |
| | CAGAAGATGATTGGAATCTTACAATGGTGGCTCTG | GCGAGTTCTTTTTCGACAAACTGCTGAACGATAAGAAC |
| | GATAATGAAATTGAAGAATCCGGAAAACAAGTTGG | TATAAAATCCGTCAGCAAGTGGTTAAGCGTGAGAAATA |
| | GGAATTCTTITTCGATAAACTTCTTAATGACAAAAA | CCAGAAGGAACTGCGTGCGATTTGGAACAAGCAACTG |
| | CTACAAAATACGCCAGCAAGTAGTTAAAAGAGAA | GAACTGAACGAGGACCTGAACAAACTGAACGAGGATC |
| | AAGTATCAAAAAGAGCTGCGAGCTATTTGGAATAA | CGGCGCTGCTGGAGCGTATCGCGAAGGAACTGTACCCG |
| | GCAACTTGAACTTAATGAAGACCTTAATAAATTAA | ACCCAGACCGAGTTCAAAGGTCCGAAGTATAAAGAAA |
| | ACGAAGACCCAGCATTACTGGAAAGAATAGCAAA | TTACCAGCAACGACCTGTACCACGTTTTTGCGAACGAC |
| | GGAGCTGTATCCTACCCAAACTGAATTTAAAGGGC | ATCATTTACTATCAGCGTGATCTGAAAAGCCAAAAGAG |
| | CTAAATATAAAGAAATCACATCTAATGACCTTTAT | CCTGATCGACGATTGCCGTTACGAGAAGAAGAAGTATT |
| | CATGTATTTGCCAATGACATTATTTATTATCAAAGA | TCGATAAGAACCTGGGTAAAGAGGTGATCCAAGGCTAT |
| | GACCTGAAATCCCAAAAGAGCTTGATTGATGATTG | AAGGTTGCGCCGAAAAGCAGCCCGGAATTTCAGGAGTT |
| | TCGTTATGAAAAGAAAAAGTACTTTGACAAAAATC | CCGTATTTGGCAAGACATCAACAACATTAAGGTGATCG |
| | TTGGCAAAGAAGTAATTCAGGGCTATAAAGTTGCT | AGAAGGAAAAAGAGATCGGTGGCAAACTGTATCCGGA |
| | CCAAAATCAAGTCCTGAATTCCAGGAGTTTCGCAT | CATTAACGTTACCGATGAGTACGTGAACAACGAAGTTA |
| | TTGGCAGGACATAAATAATATTAAGGTTATTGAAA | AGGCGCGTATCTTCCAACTGCTGGATAGCAAGAAAGAA |
| | AAGAGAAAGAAATTGGTGGAAAACTCTATCCTGAC | GTGAGCGAGAGCCAGATTCTGAAAACCATCGACAAGA |
| | ATTAACGTAACTGATGAATATGTAAACAATGAAGT | AACTGAAGCCGACCGCGTTTAAAATTAACCTGTTCGCG |
| | AAAAGCCCGCATCTTCCAGTTGTTGGATTCAAAAA | AACCGTGACAAGCTGAAGGGTAACGAGACCAAAAGCC |
| | AAGAAGTGTCCGAATCCCAAATTCTTAAAACAATT | TGTTCCGTAGCTACCTGGAGCAGTGCGGCCGTGAAAAC |
| | GATAAAAAGCTAAAACCGACAGCATTTAAAATTAA | CTGCTGAACGACCCGGATAAATTTTATAAGCTGTGGCA |
| | CTTATTTGCAAACAGGGATAAACTAAAGGGCAACG | CATTCTGTACAGCATCAACGGCAAGGATGCGGAGAAA |
| | AAACTAAATCATTATTTCGTAGTTATCTTGAACAGT | GGCATCCGTGCGGCGCTGAAAAACCCGAAGAACGAGT |
| | GTGGTCGTGAAAATTTGCTTAATGACCCTGACAAA | TCGACCTGAGCGCGGAAGTTATTGAGGAACTGGCGAGC |
| | TTTTACAAATTATGGCATATACTGTACTCAATCAAT | CTGCCGGAATTTAGCAACCAATACGCGGCGTATAGCAG |
| | GGTAAGGATGCTGAAAAAGGTATAAGGGCTGCCTT | CAAGGCGATCCACAAACTGCTGCCGCTGATGCGTAGCG |
| | AAAAAACCCAAAAAATGAATTTGATCTTTCCGCTG | GTGATCACTGGAACCACCAGAGCATTAGCCAGAAGATC |
| | AGGTAATTGAGGAACTGGCAAGTTTACCCGAATTT | CAAGACCGTATTAACAAAATCATTACCAGCGAGGAAG |
| | TCTAATCAGTATGCTGCCTACTCCTCCAAAGCCATT | ACGAGGAAATCGATAACTATACCCGTGACCAGATTACC |
| | CATAAATTATTACCATTAATGCGTTCCGGTGATCAT | AACTACTTCAAGAGCCAAAAGAACAAAGATATCTGGG |
| | TGGAACCATCAAAGCATTTCTCAAAAAATCCAGGA | AATGCGAGCTGGAAGACTTTAAAGGTCTGCCGGTGTGG |
| | CCGAATTAATAAAATCATCACAAGTGAAGAGGATG | CTGGCGTGCTACACCGTTTATGGCAAGCACAGCGAAAA |
| | AAGAAATTGATAATTACACGAGAGACCAAATTACC | AGATAAGAAAAGCTGGAAAAGCTGGAAGGAGATCGAC |
| | AACTATTTTAAAAGTCAAAAAAACAAAGATATATG | GTGATGAAGCTGGTTCCGAACAACAGCCTGCGTAACCC |
| | GGAATGTGAACTTGAAGATTTTAAGGGGCTTCCTG | GATCGTGGAGCAAATTGTTCGTGAAACCCTGCACGTGG |
| | TCTGGCTTGCTTGCTACACTGTTTATGGGAAACATT | TTCGTGATGCGTGGGAGAAATACGGTCAGCCGGACGAA |
| | CAGAGAAAGATAAAAAATCATGGAAGTCTTGGAA | ATCCACATTGAGATGAGCCGTGAACTGAAAAACCCGAA |
| | AGAAATAGATGTTATGAAATTAGTTCCAAACAATA | GGATGAGCGTGAACGTATTAGCGAAATCCAGAACAAG |
| | GTTTAAGAAATCCTATTGTTGAGCAAATTGTTAGA | AACCGTGAGGAAAAAGAGCGTATCAAGAAACTGCTGT |
| | GAAACACTGCACGTAGTAAGGGATGCTTGGGAAA | TCGAACTGAAAGAGGGTAACCCGAACAGCCCGATCGA |
| | AATACGGACAACCGGATGAAATCCACATTGAAATG | CATTAACAAGTTTCGTCTGTGGAAAAACAACGGTGGCA |
| | AGCAGGGAGTTGAAAAATCCCAAAGATGAACGAG | AGGAAGCGCAAGAGAAATTTGACAACCTGTTCAACAA |
| | AACGTATTTCAGAAATACAAAATAAAAACCGTGAA | CAAAGATGAAGTGAGCGTTAGCGGTGACGAAATCAAG |
| | GAAAAAGAAAGGATCAAAAAACTATTATTTGAATT | AAATATCGTCTGTGGGCGGATCAGAACCACACCAGCCC |
| | GAAGGAGGGAAATCCCAACTCTCCTATTGACATCA | GTACACCGGCAAGCCGATCCCGCTGAGCAAACTGTTCA |
| | ACAAATTTCGTTTATGGAAAAACAATGGAGGTAAA | CCCTGGAGTACGAAATTGAGCACATCATTCCGCAAAGC |
| | GAAGCACAAGAAAAATTTGATAACCTTTTCAATAA | CGTATGAAGAACGACAGCATGAGCAACCTGGTGATCA |
| | CAAAGATGAAGTTTCTGTTTCAGGTGATGAGATAA | GCGAAGCGGCGGTTAACGACTTTAAGGATCGTTGGCTG |
| | AGAAGTACCGGTTATGGGCTGATCAAAATCACACC | GCGCGTCCGCTGATCGAGAAATATGGTGGCACCCCGAT |
| | TCACCTTATACCGGCAAACCTATCCCATTAAGTAA | TGAACACAACGGTCAGACCTTTACCCTGCTGAACCAAG |
| | ATTATTTACGCTTGAATATGAAATAGAACACATCA | AGGAATTCGAGAAGCACTGCAACAAAACCTTTCAGAAC |
| | TCCCCCAATCAAGAATGAAAAATGACTCAATGAGT | CAACGTGGCAAGCTGAAAAACCTGCTGCGTGAGGAAG |
| | AATCTGGTTATATCTGAAGCGGCAGTAAACGACTT | TGCCGGACGATTTCGTTGAACGTCAGATCAACGACAAC |
| | CAAAGATAGATGGCTTGCACGACCACTGATCGAAA | CGTTACATTACCCGTAAACTGGGTGAACTGCTGGCGCC |
| | AATATGGAGGTACTCCCATTGAACATAATGGGCAA | GGCGGCGAAAGCGGATGAGGGTATCGTGTTTACCACCG |
| | ACATTTACATTGCTGAACCAAGAAGAATTTGAAAA | GCAGCATTACCAACGAACTGAAGGACAAATGGGGCTTC |
| | GCATTGCAACAAAACTTTCCAAAATCAACGGGGTA | CACACCCTGTGGCGTGAGCTGATGAAACCGCGTTTTGA |
| | AACTTAAGAATCTGCTCAGAGAAGAAGTCCCTGAC | ACGTCTGGAGCAGATCCTGCAAAAGAAACTGGTGGTTC |
| | GATTTTGTTGAAAGGCAAATAAATGATAACAGGTA | CGGACGAAAAGGATACCAACAAATTTCACTTCAACGAT |
| | CATTACCAGAAAATTGGGCGAATTACTTGCTCCGG | CCGGAGCCGGGTAACCCGGTGGACATTAAGCGTATCGA |
| | CAGCCAAAGCTGATGAAGGTATTGTTTTTACTACA | TCACCGTCATCATGCGCTGGATGCGCTGATTGTTGCGG |
| | GGTTCTATCACAAACGAATTAAAAGATAAATGGGG | CGACCACCCGTGCGCACATTAAATACCTGAACAGCCTG |
| | GTTCCATACATTATGGCGTGAATTGATGAAACCCA | AACAGCCACAAGAAACGTGAACCGTACAAGTATCTGG |
| | GATTTGAACGGTTAGAACAAATTCTACAAAAAAAA | CGAACAAAGGCGTGCGTGATTTTATCCAACCGTGGCCG |
| | TTAGTTGTTCCAGATGAAAAAGACACTAATAAATT | GACTTCACCGCGGAAGTGAAGAGCCAGCTGAAACGTCT |
| | TCATTTCAATGACCCGGAACCTGGCAATCCTGTAG | GATTGTGAGCCACAAGGTTAACTGCCAGTATGATCCGG |
| | ATATTAAACGAATTGATCACCGGCATCATGCATTG | AACACCCGGAGAAAAGCGGTGTGATCAGCAAGCCGAA |
| | GATGCATTAATTGTTGCCGCAACAACGCGTGCTCA | AAACCGTTTCAAGAAATGGGTGAACCGTGATGGCGTTT |
| | TATTAAATACCTTAATTCACTTAATTCCCATAAAAA | GGAAGAAAGAGTACCAGTGGCAAAAGGACAACGAAAA |
| | GCGTGAACCTTACAAGTATTTAGCAAACAAAGGTG | CTGGTGGGCGATTCGTAAGAGCATGTTTAAAGAGCCGC |
| | TGAGGGATTTTATACAACCATGGCCTGATTTTACAG | TGGGTATGATCTACCTGAAGGAAATCAAAGAGGTGTCT |
| | CGGAAGTAAAAAGTCAATTGAAACGCCTTATCGTA | CTGAAGAAAGCGCTGGAGATCCAGGCGGAACGTCAAA |
| | TCTCATAAAGTAAATTGCCAATATGATCCCGAACA | AAGGTATTAAGGACCACACCGGCCGTCCGCGTGACTAC |
| | CCCGGAAAAATCCGGTGTAATTTCAAAACCCAAAA | ATCTATGATAAGCTGGCGCGTCAGGAGATTCGTTTCCT |
| | ATAGATTCAAAAAATGGGTAAACCGGGATGGCGTT | GCTGGAAGACAAATGCGGTGGCGATATCAAGCAGGCG |
| | TGGAAAAAAGAATACCAATGGCAAAAAGACAATG | GAAAAACAAAGCAGCACCCTGAAAGATAGCAAGAGCA |
| | AAAATTGGTGGGCTATAAGAAAGTCTATGTTCAAA | ACCCGATTAAGAAAGTGCGTGTTGCGTTTTTCAAAGAG |
| | GAACCTTTGGGAATGATATATTTAAAAGAAATCAA | TACGCGGCGAGCCGTGTGCCGGTTGACAACAGCTTCAC |
| | AGAAGTTTCCCTTAAAAAAGCATTAGAAATACAAG | CTATAAGAAAATTAAGGCGATCCCGTACGCGGAAAAA |
| | CTGAAAGGCAAAAAGGGATAAAAGACCACACCGG | ATCATTAACCGTTGGGAGGAATGGGAGCAGGATGGTA |
| | AAGACCAAGAGATTACATTTATGATAAACTTGCAA | AAAACGAAAAGGGCCAAAAATTCCCGAACGACATCAC |
| | GGCAGGAAATTCGATTCTTACTTGAAGATAAATGC | CAAGTGGCCGATTGAATTTCTGCTGAAGAAACACCTGG |
| | GGTGGAGATATAAAGCAAGCAGAAAAGCAATCCA | ATGAGTATAAAACCAGCAACGGTAACCCGGACCCGAA |
| | GTACTTTAAAAGATTCCAAGAGCAATCCAATTAAA | CACCGCGTTCACCGGTGAAGGCTACGAGGCGCTGACCA |
| | AAAGTAAGAGTCGCCTTCTTTAAAGAATATGCTGC | AGAAAAACGGTGGCCAGCCGATCAAGAAAGTTACCAC |
| | AAGTAGAGTTCCAGTTGATAATTCGTTTACATACA | CTATGAAAGCAAGAGCGCGCCGATCAAGTTTAACGGTA |
| | AAAAAATCAAGGCCATTCCATATGCTGAAAAAATC | AAATTCTGGAGACCGATAAAGGTGGCAACGTGTTTTTC |
| | ATTAATAGATGGGAAGAATGGGAGCAAGATGGAA | GTTATTGCGAAGGATAAACACACCGGCAAGCACCTGGA |
| | AAAATGAGAAAGGTCAAAAATTTCCCAACGATATA | CTGGTACACCCCGCCGCTGTATAGCAACGAGGCGGAGG |
| | ACAAAATGGCCCATTGAATTTTTACTTAAAAAGCA | AAGGTAAGGAGCGTGGCATCATTAACCGTCTGATCAAC |
| | CTTGGATGAGTATAAAACATCAAATGGTAATCCTG | CGTGAGCCGATTGCGGAAGACCAGGAAGACCTGGAAT |
| | ACCCCAATACTGCTTTTACAGGAGAAGGCTATGAA | ATATCACCCTGGCGCCGGAAGACCTGGTGTACGTTCCG |
| | GCATTAACTAAAAAGAATGGAGGGCAACCGATAA | GAGGAAGACGAGGATATTCGTAGCATCGACTGGAACG |
| | AAAAGGTAACAACTTATGAATCGAAGTCAGCACCA | GCAAGGATAAACAAAAGGTGTTCGAACGTACCTACAA |
| | ATCAAGTTTAATGGAAAGATCCTCGAAACTGATAA | GATGGTTAGCAGCACCGAAAAAGAGTGCCACTTTATTC |
| | AGGTGGAAACGTCTTTTTTGTAATTGCTAAAGATA | CGCACATCGTGGCGTATCCGATCCTGAAGACCGTTGAG |
| | AACATACGGGTAAACATTTGGATTGGTACACCCCA | CTGGGTACCAACGATAAGAGCGAAAAAGCGTGGGACG |
| | CCTTTGTATAGCAATGAAGCAGAAGAAGGCAAAG | GCAAAGTGGAGTACATTCCGAACAAGAAAGGTAAACT |
| | AAAGAGGAATTATAAATCGTTTGATTAACAGAGAA | GACCCGTAAAGATAGCGGCACCATGATCAAGGAGAAC |
| | CCCATTGCTGAAGATCAAGAGGATTTGGAATATAT | TGCGTTAAAATTAAGCTGGACCGTCTGGGTAACATCAT |
| | CACACTTGCTCCAGAGGATTTGGTATATGTTCCGG | TAAGGTGAACGGCAAACCGGTTAACCACTAG (SEQ ID |
| | AAGAAGATGAGGATATTCGGTCTATTGATTGGAAT | NO: 54) |
| | GGAAAAGACAAGCAGAAAGTTTTTGAAAGGACTTA | |
| | TAAAATGGTGAGTTCTACAGAAAAAGAATGCCACT | |
| | TTATTCCCCACATTGTTGCCTATCCAATTTTAAAAA | |
| | CAGTTGAATTAGGGACAAATGATAAATCAGAAAAA | |
| | GCATGGGATGGAAAAGTTGAATATATACCAAATAA | |
| | AAAGGGGAAATTAACCCGAAAAGATTCCGGAACA | |
| | ATGATCAAAGAAAATTGCGTAAAAATAAAATTAGA | |
| | TAGACTTGGAAACATAATTAAAGTCAATGGTAAAC | |
| | CGGTTAATCATTAA (SEQ ID NO: 53) | |
| Type II | ATGTCCAATGCCCGTCCTTCCATCCTGCCCGATGAT | ATGAGCAACGCGCGTCCGAGCATTCTGCCGGACGATCT |
| Cas_3 | CTGATCCTTGGTCTCGACATCGGTACCAACTCGGTC | GATCCTGGGTCTGGACATTGGCACCAACAGCGTGGGTT |
| | GGATGGGCTCTCATCCACTATGCCGAGAGCGAACC | GGGCGCTGATTCACTACGCGGAGAGCGAACCGCGTCAA |
| | GCGACAGCTCATCGCACTCGGATCGCGTGTATTCG | CTGATCGCGCTGGGTAGCCGTGTTTTCGAGGCGGGTAT |
| | AAGCGGGCATGGACGGTTCAATCAGTCACGGCAAG | GGATGGCAGCATCAGCCACGGCAAAGAGGAGAGCCGT |
| | GAGGAGTCACGAAACAAGAAGCGGCGGGATGCGC | AACAAGAAACGTCGTGATGCGCGTAGCCTGCGTCGTGC |
| | GGTCCCTTCGGCGGGCGACGTGGCGTCGAAAGCGT | GACCTGGCGTCGTAAGCGTCGTAAACGTCGTGTGTATA |
| | CGAAAGCGGAGGGTATACAATCTGCTTCACGAAGC | ACCTGCTGCATGAAGCGGGTCTGCTGCCGGACGCGGAT |
| | AGGGCTGCTTCCGGACGCTGACACGAACGATCCGG | ACCAACGACCCGGAGAGCATTAACGTTGCGCTGACCCG |
| | AATCGATCAACGTGGCTCTGACCCGACTCGATCGG | TCTGGATCGTGAACTGGTTAGCAAATTTGTTAGCCCGG |
| | GAACTCGTTTCCAAGTTCGTCTCGCCGGGCGATCAT | GTGACCACCGTGAAGCGCAGCTGATGCCGTATCTGGCG |
| | CGCGAGGCTCAGCTGATGCCGTACCTCGCCAGGCG | CGTCGTCGTGCGGTGGAGGAACGTGTTGAACCGGTGGT |
| | ACGCGCCGTGGAGGAGCGCGTAGAGCCTGTCGTTT | TCTGGGTCGTGCGCTGTATCACATCGCGCAGCGTCGTG |
| | TGGGTAGAGCGCTCTACCACATCGCGCAACGGCGA | GCTTCCGTAGCAACCGTCGTACCGCGATGCGTGAGGAC |
| | GGCTTCCGGTCGAATCGGCGGACGGCCATGCGAGA | GAAGATCTGGGTCAAGTGAAGAGCGCGATCGCGAGCC |
| | AGACGAAGATCTAGGGCAGGTCAAAAGCGCGATT | TGCACCACAAAATTGTTGAGAGCGAAGGCGAGATCCA |
| | GCGTCGCTGCATCACAAGATTGTTGAGTCCGAAGG | GACCCTGGGTGGCTACTTTGCGAGCCTGGATCCGCACG |
| | AGAGATCCAGACGCTTGGTGGGTACTTCGCCTCAC | AGGAACGTATCCGTACCCGTTGGACGGTCGTGACATG |
| | TCGATCCTCACGAAGAACGAATCCGTACCCGATGG | TACCTGGAGGAATTCGACAAGATCGTGGATCGTCAAAT |
| | ACGGGTCGTGATATGTACCTGGAAGAGTTCGATAA | TCCGTATCACGATGGCCTGACCAGCGAACGTGTTGAGG |
| | AATCGTTGATAGGCAGATTCCTTACCACGATGGCC | CGCTGCGTGCGGCGATTTTTGACCAGCGTCCGCTGCGT |
| | TTACGAGCGAACGGGTCGAGGCGCTGCGCGCTGCG | AGCCAAAACCACCTGATCGGTCGTTGCGAACTGGAGCG |
| | ATCTTTGATCAGCGTCCCTTGCGGTCGCAAAATCAC | TGATCAGCGTCGTTGCAGCATCGCGCTGCTGGAGTATC |
| | CTGATTGGTCGATGCGAACTAGAGCGAGATCAGAG | AGCGTTTCCGTCTGCTGCAAGCGGTGAACAACCTGCGT |
| | GCGATGCTCGATTGCCCTTCTGGAGTATCAGCGGTT | TGGCTGAGCGACGAAGGCCACGAACGTGAGCTGAGCC |
| | TCGGTTACTCCAGGCCGTGAACAATCTCCGCTGGC | GTGAGGAACGTCTGCGTCTGGTTCGTGAACTGGAGATT |
| | TTTCTGACGAAGGTCATGAACGAGAACTCTCGCGG | AAGCCGGAGCTGGCGTTTGGTAAAATCCGTACCCTGCT |
| | GAAGAACGTCTCCGTCTGGTCAGGGAGCTTGAGAT | GGGTCTGAAGCGTGGTACCGGCCGTTTCAACCTGGAAC |
| | CAAGCCGGAACTCGCATTCGGAAAGATTCGCACGC | TGGGTGGCGAGAAACGTCTGATTGGTAACCGTACCAAC |
| | TTCTCGGATTGAAGCGCGGCACAGGCCGGTTCAAT | GCGCAGCTGCGTGCGCTGTTTGAAGCGCGTTGGGAGAC |
| | CTGGAACTCGGCGGCGAGAAGCGACTCATCGGAA | CTTCACCAACGACGAACAGAGCAGCATCGTGCACGATC |
| | ATCGCACGAATGCGCAGTTGCGCGCGCTCTTCGAG | TGATGAGCATCCAAAACCCGATTGCGCTGCAGCGTCGT |
| | GCGCGGTGGGAGACGTTCACGAACGACGAGCAAT | GGTCAAGTTCGTTGGGGTCTGGATGGCGAGAAGAGCAG |
| | CGTCGATCGTGCATGATCTGATGAGCATCCAAAAC | CTACTTTGCGAACGACCTGCTGCTGGAAGATGGTTATG |
| | CCGATCGCCCTGCAGCGCAGGGGGCAAGTGAGGTG | CGCCGCTGAGCCTGCGTGCGATTCGTAAGCTGCTGCCG |
| | GGGTCTTGATGGCGAGAAGAGTAGCTATTTCGCCA | CGTCTGGAGGAAGGCATCCCGTACAGCACCGCGCGTAA |
| | ATGACCTCCTTCTCGAGGATGGCTACGCGCCCCTTT | AGAAATGTATCCGGAGAGCTTCCAGAGCAGCGTGGTTC |
| | CGCTTCGTGCGATTCGAAAGCTGCTGCCTCGACTC | TGGACCGTCTGCCGCCGCTGGCGAAAACCGATCTGGAG |
| | GAGGAAGGCATTCCGTATTCGACAGCGAGAAAGG | GCGCGTAACCCGAGCATTATGCGTACCCTGAGCGAAGT |
| | AGATGTATCCTGAATCGTTCCAATCCTCGGTCGTGC | GCGTGCGGTGGTTAACGCGATTGTTCGTCAGTACGGTC |
| | TCGATCGGCTTCCACCTCTTGCTAAGACGGACCTCG | GTCCGGGTCTGGTGCGTATTGAGCTGGCGCGTGACCTG |
| | AAGCGCGGAATCCGTCGATTATGAGGACGCTCTCC | AAGCAACCGAAACGTCGTCGTCAGGAAATCAGCCGTCA |
| | GAAGTACGAGCAGTGGTCAATGCCATCGTTCGACA | AATGCGTGAACGTGAGGGTGTTCGTGAGAAGGCGAAG |
| | GTACGGAAGGCCTGGACTCGTTCGGATTGAGCTGG | AAACGTCTGCTGGATACCGAATTTGGTGGCAGCCGTGC |
| | CTCGGGATCTGAAGCAGCCGAAGAGGCGACGCCA | GAGCCGTGCGGACATTGAGAAACTGATTCTGGCGGACG |
| | GGAAATCTCACGACAGATGCGGGAGCGAGAGGGG | AATGCGATTGGACCTGCCCGTACACCGGTCGTGGCTTT |
| | GTTCGCGAGAAGGCCAAGAAGCGCCTGCTTGATAC | GGTATGGGCGACCTGTTCGGTAGCAACCCGACCATCGA |
| | CGAGTTTGGCGGGTCGCGAGCCAGCCGAGCCGATA | TGTGGAGCACATTCTGCCGTTTAGCCGTTGCCTGGACA |
| | TCGAAAAGCTCATCCTTGCCGACGAGTGCGATTGG | ACAGCTTCCTGAACAAGACCCTGTGCGATGTGCGTGAA |
| | ACGTGCCCGTATACGGGGCGCGGCTTCGGGATGGG | AACCGTCTGGTTAAACGTAACCGTACCCCGTTTGAGGC |
| | CGATCTATTCGGATCAAATCCCACGATCGACGTGG | GTATGCGGGTCAACGTGACCGTTGGGAAGCGATCCTGG |
| | AGCACATCCTTCCCTTCAGTCGCTGTCTCGACAATT | ATCGTATTAAGAACTTCAAAAGCGATCCGCTGACCGTG |
| | CCTTCCTCAACAAGACTCTCTGTGACGTACGCGAA | CGTCGTAAGCTGGAGCGTTTTCTGCAGGAAGAGCTGAG |
| | AATCGCCTAGTGAAGCGCAATCGGACCCCGTTCGA | CAGCGCGCGTGTTGACGAATTCAGCGAGCGTGCGCTGA |
| | AGCCTATGCCGGTCAGCGCGATCGATGGGAAGCGA | GCGATACCCGTTACGCGAGCCGTCTGGTTGCGGACTTC |
| | TCCTTGATCGGATCAAGAACTTCAAGTCGGATCCG | ATGGGTCTGCTGTATGGTGGCCGTAACGACAGCGATGG |
| | CTGACGGTCCGTCGGAAGCTGGAACGATTTCTCCA | CAAGCAGCGTGTGCAAGTTAGCAGCGGCCAAGCGACC |
| | AGAGGAACTCTCGTCGGCGCGAGTCGACGAGTTCA | AGCATTCTGCGTCGTGAGTGGGGCCTGAACAGCCTGCT |
| | GCGAGCGCGCGCTTTCCGATACACGATACGCGTCG | GGGTGGCGAAGCGCGTAAAAGCCGTCTGGACCACCGTC |
| | CGTCTGGTCGCCGACTTCATGGGGTTGTTGTATGGG | ACCATGCGGTGGATGCGGTGGTTATCGCGCTGACCGGT |
| | GGACGGAACGATTCCGATGGGAAGCAGCGAGTTCC | CGCGTGAGGTTAAACGTCTGGCGGATGCGGCGAAACG |
| | AGGTCTCCAGCGGCCAAGCGACTTCGATCCTACGT | TGCGGCGGATCAGGGTAGCCACCGTCTGTTCGAGGAAG |
| | CGTGAATGGGGTCTCAACTCGCTGCTGGGCGGGGA | TGCCGTTTCCGTGGACCCACTTCCGTACCGACGTGAAC |
| | GGCTCGGAAGTCTCGACTCGATCACCGCCATCATG | GAGAAGATTCATTGCTGCGTTACCAGCCCGCGTCCGAG |
| | CGGTCGATGCCGTAGTCATCGCGTTGACTGGGCCA | CCGTCGTCTGCGTGGTCCGCTGCACGATGAAAGCCTGT |
| | CGCGAGGTGAAACGACTAGCCGACGCTGCAAAAC | ACAGCCGTCCGCTGCCGTGGTATGACAAGAAAGGCCGT |
| | GAGCGGCCGATCAAGGAAGTCATCGCCTTTTCGAG | GAGAGCCTGCGTCCGCGTATCCGTAAGCCGATTGAACA |
| | GAGGTTCCGTTTCCGTGGACTCATTTCCGCACCGAC | ACTGACCAAAGGTGAAGTTGAACGTATTGCGGACCCGG |
| | GTGAACGAGAAGATTCATTGTTGCGTGACCTCTCC | GCGTGCGTGATGCGGTTAAGACCCGTGCGGCGGAGCTG |
| | CCGACCGTCCAGGCGGCTCCGTGGGCCGCTTCACG | GCGAAGGGTCAGGGTGGCAGCGGCGACCTGAGCAAAC |
| | ACGAGAGCCTCTATTCACGCCCGCTCCCCTGGTAT | TGTTTAGCGATCCGAGCCACGCGCCGTTCCTGCGTAAC |
| | GACAAGAAGGGGAGAGAGAGTCTTCGGCCAAGGA | CGTGACGGTAGCACCACCCCGATCCGTCGTGTGCGTAT |
| | TCCGTAAGCCGATCGAACAGCTCACCAAGGGCGAG | TACCGCGAAGGTTAAACAGGCGACCCCGATTGGTGAAG |
| | GTTGAGCGAATCGCGGATCCAGGCGTTCGGGACGC | GCGTGCGTCAACGTCATGTTGCGCCGGGTAGCAACCAC |
| | GGTGAAGACCAGGGCCGCTGAACTCGCGAAAGGG | CACATGGCGATCGTGGCGATTCTGGATGAAAAGGGTAA |
| | CAAGGAGGCAGTGGGGATCTCAGTAAGCTCTTCTC | CGAGAAACGTTGGGAAGGCCACGTGGTTACCATGCTGG |
| | CGACCCGAGCCACGCTCCGTTTCTGCGAAACCGTG | AGGCGGTGCTGCGTAAGGGTCGTGGCGAACCGGTTATC |
| | ATGGTTCGACCACCCCGATTCGGCGCGTCCGGATT | CAGCGTGACTGGGGTAAAGGCCAAAAGTTCAAATTTAG |
| | ACCGCGAAGGTCAAGCAGGCCACGCCGATCGGAG | CCTGCGTAGCGGTGACTGCATTTGGAACTGCGATACCG |
| | AAGGTGTTCGTCAACGTCATGTCGCGCCCGGCTCG | GCCGTATCATGCACGTGAAAGCGGTTAGCGCGGGTGTG |
| | AATCATCACATGGCGATCGTTGCAATTCTGGACGA | GTTGAAGGCCTGGAAGTGAACGACGCGCGTACCGCGGT |
| | GAAGGGGAATGAGAAGCGCTGGGAAGGTCATGTC | GGATGTTCGTCGTGCGGGTGTGGTTGGTGGCCGTTACA |
| | GTCACGATGCTGGAGGCCGTGCTCCGGAAGGGGCG | CCGCGAGCCCGGAGCGTCTGCGTAAGGACGCGTTCGTG |
| | TGGGGAGCCGGTGATCCAACGGGATTGGGGAAAG | CGTTGCGTGGTTGATCCGCTGGGCAAAGTTATCCCGAG |
| | GGGCAAAAGTTCAAGTTTTCGCTTCGATCGGGAGA | CAACGAATAG (SEQ ID NO: 56) |
| | CTGCATCTGGAATTGCGACACCGGGCGGATTATGC | |
| | ATGTCAAGGCGGTTTCAGCGGGTGTCGTGGAAGGC | |
| | CTCGAAGTGAACGATGCCCGGACAGCGGTTGATGT | |
| | GAGAAGAGCCGGCGTCGTTGGAGGGCGCTATACG | |
| | GCAAGCCCAGAGCGACTTCGAAAAGACGCTTTCGT | |
| | TCGCTGTGTCGTGGACCCACTCGGGAAGGTCATAC | |
| | CATCCAATGAGTGA (SEQ ID NO: 55) | |
| Type II | ATGACATATATTTTGGGTTTAGACCTCGGCATTTCA | ATGACCTACATCCTGGGTCTGGACCTGGGCATTAGCAG |
| Cas_4 | TCGGTCGGCTTTGCCGGCATTGATCATAATGGGGA | CGTTGGTTTCGCGGGCATCGATCACAACGGTGACAACA |
| | TAATATTCTTTTCGCAAATGCCCATGTATTTGATAA | TTCTGTTCGCGAACGCGCACGTGTTTGATAAGGCGGAA |
| | GGCAGAGGTTGCCAAAACCGGCGCATCGCTGGCTG | GTTGCGAAGACCGGTGCGAGCCTGGCGGAACCGCGTCG |
| | AACCACGGCGTAATGCCCGCCTGACCCGCCGCCGC | TAACGCGCGTCTGACCCGTCGTCGTATCGAACGTAAAG |
| | ATCGAACGGAAAGCCCGGCGCAAATCACGTATTAA | CGCGTCGTAAGAGCCGTATCAAGAACCTGTTTGATAAG |
| | AAATTTATTTGATAAATATGGCTTGGATGTGGAGG | TACGGTCTGGACGTTGAAGCGATTGATCGTCCGCCGAG |
| | CGATTGACCGCCCGCCTTCCCCGGATCGTCAATCG | CCCGGACCGTCAGAGCGTGTGGGATCTGCGTCGTGTTG |
| | GTATGGGATTTGCGACGGGTTGGCTTGTCAAAAAA | GTCTGAGCAAGAAACTGAACAGCGGCCAGTGGGCGCG |
| | ATTAAACTCGGGCCAATGGGCACGTGCGTTATTTC | TGCGCTGTTCCACCTGGCGAAAAACCGTGGTTTTCAAA |
| | ATTTGGCCAAAAACCGTGGCTTTCAATCCAACCGA | GCAACCGTAAAGATAAAGCGGATGGTGTGGGTACCGG |
| | AAGGATAAGGCAGACGGGGTCGGCACTGGTAAAT | CAAGAGCGACACCGATAACGGCCGTATGCTGAGCGCG |
| | CGGATACCGATAACGGCCGGATGCTGTCGGCGATT | ATCAGCGACCTGAAGAAAAACCTGGCGGAAAGCGATC |
| | TCCGATTTGAAAAAAAATCTGGCGGAGAGCGACCA | ACGAGACCATTGGTAGCTACCTGAGCACCCTGGACAAG |
| | TGAAACAATCGGATCTTATTTATCCACGCTGGATA | AAACGTAACGGCGACGATGACTATAGCAAAACCGTGC |
| | AAAAACGCAACGGGGATGATGATTATTCCAAAACC | ACCGTGATATGATCCGTGACGAAGTTAGCCTGCTGTTC |
| | GTGCATCGGGATATGATCCGGGATGAGGTTTCCTT | CAGCGTCAACGTAGCTTTGACAACCCGCACGCGGGTAC |
| | ACTATTTCAACGGCAACGATCCTTTGATAACCCGC | CGAGCTGGAACAGGCGTTCTGCAAGGTGGCGTTTTACC |
| | ATGCCGGAACGGAGTTGGAACAGGCGTTTTGTAAG | AGCGTCCGCTGCAAAGCACCATCGAACTGATTGGCAAC |
| | GTTGCCTTTTATCAACGCCCATTGCAGTCCACCATC | TGCAGCATCTTCCCGGACGAGAAGCGTGCGCCGAAACA |
| | GAATTAATCGGTAATTGCAGTATTTTCCCGGATGA | CGCGTATAGCAGCGAGGAATTTCTGGCGTGGAGCCGTC |
| | AAAACGGGCGCCGAAACATGCCTATTCAAGTGAAG | TGAACAACCTGCGTCTGCTGACCCCGAGCGGTAAGAAA |
| | AATTTTTGGCCTGGAGCCGGCTGAATAATTTACGCT | AAGGAGCTGACCACCGGCCAGAAAGAAAAGGCGATCG |
| | TACTCACCCCGTCCGGCAAAAAAAAGGAATTGACG | AGCTGACCAAGCAATACAAAAAGGGTGTTACCTTCGCG |
| | ACAGGTCAAAAAGAAAAGGCCATAGAGCTGACCA | CGTCTGCGTCGTGCGCTGGACATTGATGACCAGTACCG |
| | AGCAGTATAAAAAAGGCGTAACCTTTGCCCGCCTG | TTTTAACCTGTGCCACTATCGTAACACCATGGACGGCC |
| | CGCCGTGCATTGGACATCGATGATCAATATCGGTT | CGAGCGACTGGGATACCATCCGTGATAAAAGCGAAAA |
| | TAATCTATGCCATTACCGCAATACCATGGATGGCC | GCAGGTGCTGATTCAATTCCCGGGTTATCACGCGATGC |
| | CATCGGATTGGGACACAATCCGGGATAAATCGGAA | GTGATCAACTGAGCGACCTGGGCGCGGATGACATCCAC |
| | AAACAGGTTTTAATCCAATTTCCGGGCTATCACGC | TTCACCGAGCTGCTGGCGAACCGTGACCAGTACGATGA |
| | CATGCGGGATCAATTATCCGACCTCGGTGCGGATG | CACCATCCAAATTCTGAGCTTTTATGAGGATGAAGCGG |
| | ATATCCATTTTACCGAATTATTGGCCAACCGGGATC | ACATCCTGAGCCGTCTGAGCGATCTGGGTCACCTGCCG |
| | AATATGATGACACCATCCAAATTTTGAGTTTTTATG | GAAGTTATTGAGAAACTGAAGTACCTGGACTTCAGCCG |
| | AGGATGAGGCCGATATCCTGTCCCGTCTATCGGAC | TACCATCGATCTGAGCCTGAAAGCGGTGAAGCAGATTC |
| | CTGGGCCATTTGCCTGAAGTCATCGAAAAACTAAA | TGCCGTATATGAAAAAGGGCTACGACTATGCGACCGCG |
| | ATATCTTGATTTTTCCCGAACCATCGATCTGTCATT | CGTGATATGGCGGGTCTGAAACCGAAGAACACCAAAA |
| | AAAGGCGGTGAAACAGATCCTGCCTTATATGAAAA | GCGGCAACAAAAAGCTGCTGAGCCCGTTTGACAGCACC |
| | AGGGGTATGATTATGCCACGGCAAGGGATATGGCC | AAAAACCCGGTGGTTGATCGTTGCCTGGCGCAAAGCCG |
| | GGGCTTAAGCCAAAAAATACAAAAAGCGGGAATA | TAAGGTGGTTAACGCGGTTATCCGTCGTCACGGTCTGC |
| | AAAAACTGTTATCCCCGTTTGATTCGACAAAAAAT | CGGACTACATCCACATTGAACTGAGCCGTGATCTGGGC |
| | CCGGTTGTTGACCGGTGCCTTGCCCAATCCAGAAA | CGTAGCAAAAAGGAGCGTGATAAGATCGACCGTCGTAT |
| | GGTTGTTAATGCGGTTATTCGTCGCCATGGACTTCC | TGAAAAGAACCGTCGTTACAAAGAGGACCTGCGTCAGC |
| | CGATTATATTCATATCGAATTATCACGTGACCTGGG | ACGCGGCGGAACTGCTGGATCGTGAGCCGAGCGGCGA |
| | CCGATCAAAAAAAGAACGGGATAAAATTGATCGC | GGAATTCCTGAAGTATCGTCTGTGGAAAGAGCAGGACG |
| | CGTATTGAAAAAAATCGCCGGTATAAAGAAGATCT | GTATCTGCCCGTACAGCGGCAGCTATATTGAGCCGGAT |
| | GCGTCAGCATGCCGCCGAATTATTGGATCGGGAGC | GAGTGGGCGAGCCCGACCGCGGTTCAAATCGACCACAT |
| | CAAGCGGGGAAGAATTTTTAAAATACCGCCTTTGG | TCTGCCGTTTAGCCGTAGCTACGATAACAGCTATATGA |
| | AAAGAACAAGACGGTATATGCCCCTATTCCGGCAG | ACAAAGTGCTGTGCACCGCGAGCGCGAACCAAGAAAA |
| | TTATATCGAACCGGATGAATGGGCATCGCCCACGG | GGGTAACAAGACCCCGTACGAGTGCTGGGGCCAGATG |
| | CGGTACAAATTGATCATATCCTGCCCTTTTCAAGAT | GATGACCTGTGGCCGGCGATCATGGCGCAAGCGGACA |
| | CCTATGACAATAGTTACATGAATAAGGTGCTTTGC | AGCTGCCGAAAAAGAAACGTGATCGTATTCTGAACAAA |
| | ACGGCCAGCGCAAATCAGGAAAAGGGGAATAAAA | CACTTCAACGAGCGTGAACAGGAGTTTAAGACCCGTCA |
| | CCCCGTATGAATGCTGGGGTCAGATGGATGATCTA | CCTGAACGACACCCGTTACATCGCGCGTCAGCTGCGTC |
| | TGGCCCGCGATTATGGCACAGGCGGATAAACTGCC | AAAACATTAGCGAACAACTGGATCTGGGTGACGGCAA |
| | TAAGAAAAAACGGGATCGTATATTAAACAAACATT | CCGTGTTCGTGTGCGTAACGGTTATATCACCAGCTTCCT |
| | TTAATGAACGGGAACAGGAATTCAAAACCCGTCAT | GCGTGGTATTTGGGGCCTGCAGGACAAAACCCGTGACA |
| | TTAAATGATACCCGCTATATTGCCCGCCAGCTTCGC | ACGATCGTCACCACGCGATCGATGCGATCATTGTGGCG |
| | CAAAATATTTCTGAACAACTGGATCTGGGGGATGG | TGCACCACCGAAGGTATTATGCAGCAAGTTACCCAATG |
| | CAATCGGGTGCGTGTGCGCAATGGATATATCACAT | GAACAAATACGACGCGCGTCGTAAAGATAAGGAGCCG |
| | CCTTTTTACGTGGGATATGGGGATTACAGGATAAA | TATTTCCCGAAGCCGTGGGACGGCTTTCGTAGCGATGT |
| | ACCCGTGACAATGACCGCCATCATGCCATTGATGC | TTGGGACGCGTACCACGCGGTTTTCGTTAGCCGTCTGCC |
| | GATTATTGTTGCCTGCACCACCGAAGGTATTATGC | GGATCGTAGCGCGACCGGTGCGATGCACAAGGAGACC |
| | AACAGGTCACCCAATGGAATAAATATGATGCCCGA | GTGCGTAGCCTGCGTACCGATGACGATGGCAACGACGT |
| | CGCAAGGATAAAGAACCCTATTTCCCCAAACCATG | GGTTGTGCAGCGTATCCCGATTACCGACCTGAGCAAAG |
| | GGATGGTTTTCGATCCGATGTGTGGGATGCCTATCA | CGAAGCTGGAAGATATCGTGGACAAAGATACCCGTAA |
| | TGCGGTGTTTGTTTCCCGCCTACCCGACCGGTCGGC | CACCCGTCTGTATAACACCCTGAAGACCCGTATGGAGA |
| | CACCGGGGCGATGCATAAAGAAACGGTACGAAGC | AACACGGTTACAAAGCGGACAAGGCGTTCGCGAAGCC |
| | CTGCGCACCGATGATGATGGTAATGATGTCGTGGT | GATCTATATGCCGACCAACAGCGATAAACAGGGTCCGC |
| | CCAACGTATCCCGATTACCGATCTTTCCAAGGCCA | CGATCAAGCGTGTGCGTATTGTTACCAACAAACAAAAG |
| | AGTTAGAGGATATCGTTGATAAAGATACCCGCAAC | GACATTGTGCTGCCGAAACGTGGTGGCGGTGTTGCGGA |
| | ACCAGGCTGTACAATACCCTTAAAACCCGGATGGA | CCGTGCGAACATGGTTCGTGTGGATGTTTTTGAAAAGG |
| | AAAACATGGGTATAAGGCGGATAAGGCATTTGCCA | GCGGTAACTTCTTTCTGTGCCCGGTTTACACCGACCAGA |
| | AACCAATCTACATGCCCACCAACTCGGATAAACAA | TCATGCGTGGTGAGCTGCCGATGCGTCTGGTGAAAGCG |
| | GGCCCGCCGATTAAACGGGTGCGTATTGTCACCAA | AGCAAGGATGAAAGCGAGTGGCCGGAAATTACCGATG |
| | TAAGCAAAAGGATATTGTCTTGCCCAAACGCGGGG | AGTATGACTTCAAGTTTAGCCTGTACAAAAACGACTAT |
| | GCGGAGTCGCCGATCGGGCAAATATGGTCCGGGTG | GTGAAGATCAAGAAAAAGAGCAAAGGTGAAATTGTTG |
| | GATGTCTTTGAAAAAGGGGGGAATTTTTTCCTTTGC | AACTGGAGGGTTACTATAACGGCACCGATCGTGCGACC |
| | CCGGTATATACCGATCAAATTATGCGGGGCGAACT | GCGAGCATCAGCCTGCGTATTCACGACAACGATCAGGA |
| | GCCGATGCGCCTGGTAAAGGCCAGTAAAGACGAAT | CGTGGGTAAAAACGGCATGATCCGTGGTATTGGCGTTT |
| | CCGAATGGCCGGAAATTACCGATGAGTATGATTTT | ACCGTCTGCTGAGCTTCGAGAAGTACACCGTGAGCTAT |
| | AAATTCAGCCTGTATAAAAATGACTATGTCAAAAT | TTTGGTCAGCTGAGCCGTGTGAACCAAGGCGGTCGTCC |
| | AAAGAAAAAATCCAAAGGAGAGATTGTAGAATTA | GGGCGTTGCGTAG (SEQ ID NO: 58) |
| | GAGGGGTATTATAATGGTACTGATCGTGCAACGGC | |
| | CAGTATAAGCCTACGCATTCATGACAATGATCAGG | |
| | ATGTCGGTAAAAACGGCATGATCAGAGGCATTGGC | |
| | GTTTACCGACTGTTATCCTTTGAAAAATATACTGTG | |
| | AGTTACTTTGGGCAATTATCACGGGTAAACCAAGG | |
| | GGGTCGACCTGGCGTGGCGTAG(SEQ ID NO: 57) | |

In some embodiments, the Type II endonuclease of the disclosure is catalytically active.

In some embodiments, the Type II endonuclease of the disclosure is catalytically dead e.g. by introducing mutations in one or more of the RuvC domains.

In some embodiments, the Type II endonuclease of the disclosure is a Type II nickase.

The Type II endonucleases of the disclosure can be modified to include an aptamer.

The Type II endonuclease of the disclosure can be further fused to domains, e.g. catalytic domains to produce dual action Cas proteins. In some embodiments, a Type II endonuclease is further fused to a base editor.

gRNAs for Class 2 Type II CRISPR-Cas RNA-Guided Endonucleases

The present disclosure provides DNA-targeting RNAs that direct the activities of the novel Type II endonucleases of the disclosure to a specific target sequence within a target DNA. These DNA-targeting RNAs are referred to herein as “gRNAs” or “gRNAs” Generally, as provided herein, a Type II gRNA comprises a first segment (also referred to herein as a “targeter-RNA”, a “DNA-targeting segment” or a “DNA-targeting sequence”) and a second segment (also referred to herein as a “activator-RNA”, a “activator-RNA” or a “protein-binding sequence”). Also provided herein are nucleotide sequences encoding the Type II gRNAs of the disclosure.

i. Targeter-RNA

The targeter-RNA of a Type II endonuclease gRNA of the disclosure comprises a nucleotide sequence that is complementary to a sequence in a target DNA (targeting sequence of the gRNA; DNA-targeting sequence; spacer sequence). The targeter-RNA can interchangeably be referred to as a crRNA. The targeter-RNA of a gRNA interacts with a target DNA in a sequence-specific manner via hybridization (i.e., base pairing). As such, the nucleotide sequence of the targeter-RNA may vary and determines the location within the target DNA that the gRNA and the target DNA will interact. The targeter-RNA of a subject gRNA can be modified (e.g., by genetic engineering) to hybridize to any desired sequence within a target DNA.

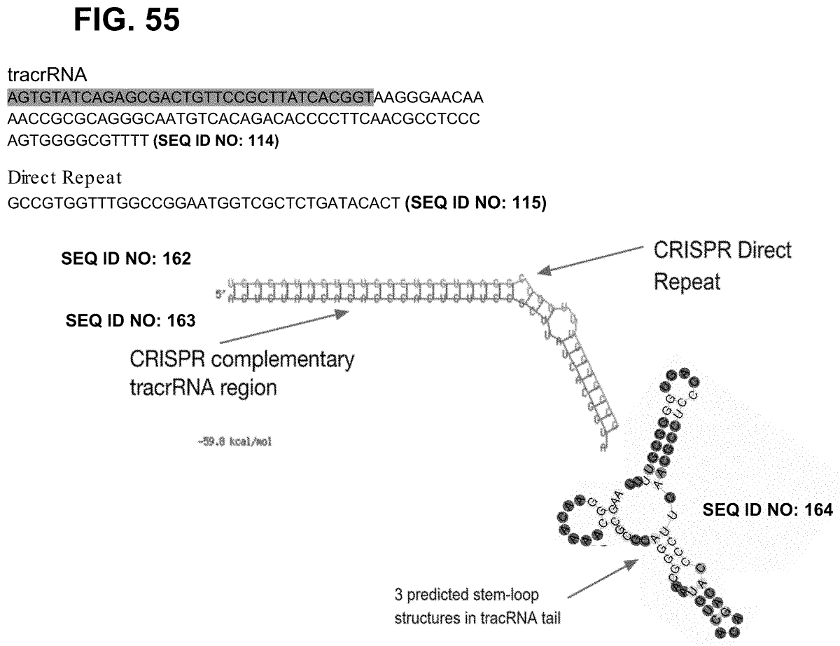

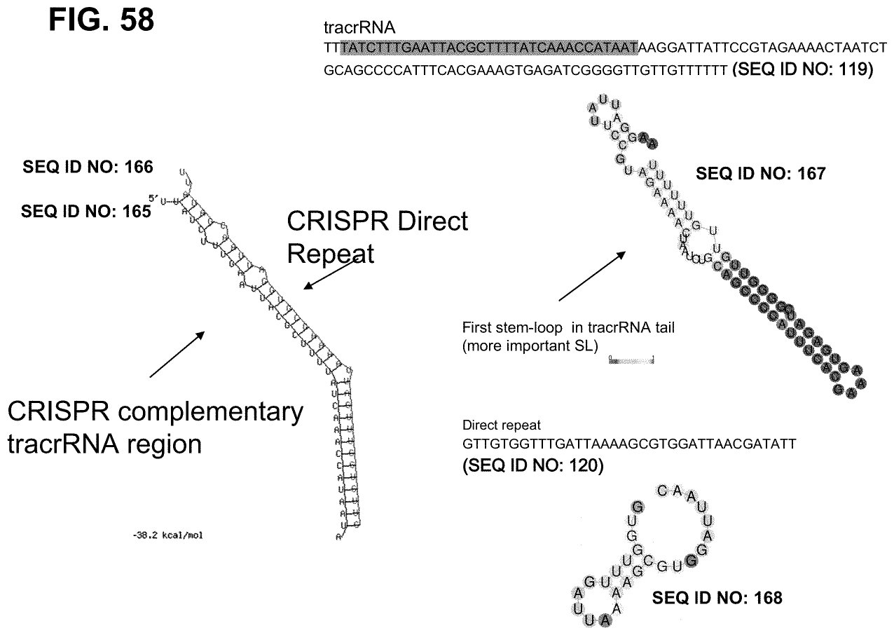

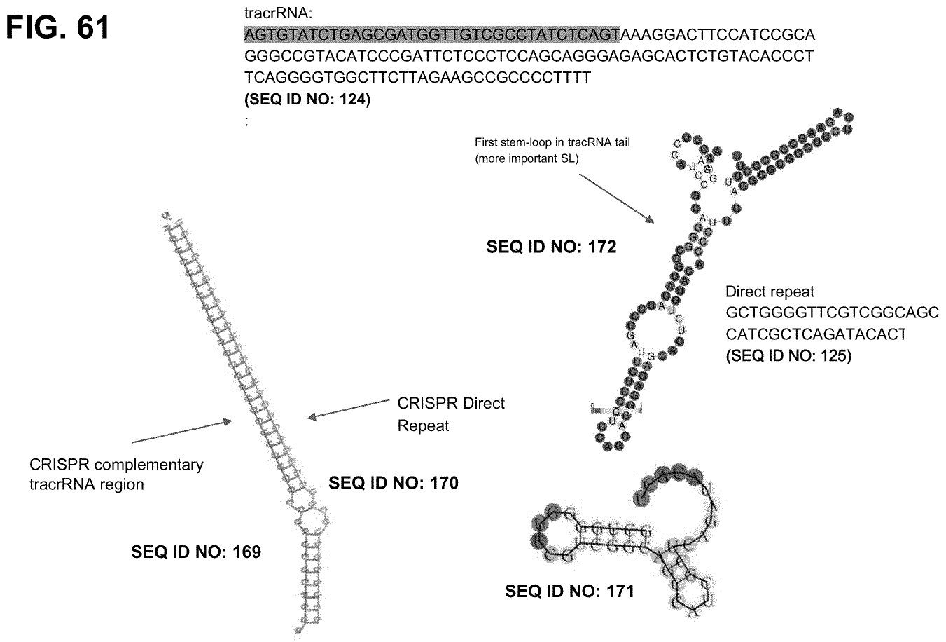

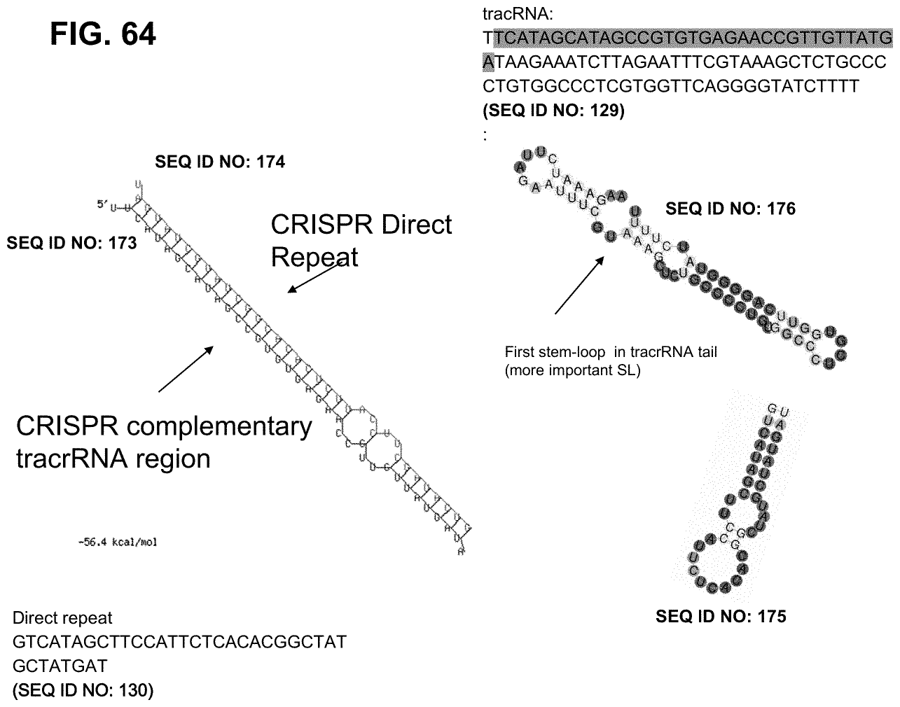

Generally, a naturally unprocessed pre-crRNA of Type II comprises a direct repeat and an adjacent spacer (the portion of the crRNA that allows for targeting to a DNA molecule). In some embodiments, direct repeats (partial sequence or entire sequence) from unprocessed pre-crRNA are included into the Type II gRNAs of the disclosure, and improve gRNA stability. Exemplary direct repeat sequences include SEQ ID NO: 115, 120, 125, and 130. It is noted that while the exemplary sequences are provided in DNA nucleotides, it is understood that this DNA can then be transcribed into RNA. Accordingly the mature guides of disclosure may incorporate the entire or partial sequence of the exemplary direct repeat sequences provided herein; the guides may be composed of DNA nucleotides, analogous RNA nucleotides, or a combination of DNA and RNA nucleotides. Exemplary predicted secondary structures of the pre-crRNAs of the Type II endonucleases of the disclosure are presented in FIGS. 55, 58, 61, and 64.

The targeter-RNA can have a length of from about 12 nucleotides to about 100 nucleotides. For example, the targeter-RNA can have a length of from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, or from about 12 nt to about 19 nt. For example, the targeter-RNA can have a length of from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 19 nt to about 70 nt, from about 19 nt to about 80 nt, from about 19 nt to about 90 nt, from about 19 nt to about 100 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, from about 20 nt to about 70 nt, from about 20 nt to about 80 nt, from about 20 nt to about 90 nt, or from about 20 nt to about 100 nt.

In some embodiments, the gRNAs of the disclosure include a portion of, or the entirety of the naturally occurring direct repeat sequences which can be incorporated into the engineered gRNAs of the disclosure. Exemplary Type II naturally occurring direct sequences are provided herein, and include SEQ ID NO: and 115, 120, 125, and 130. FIGS. 55, 58, 61, and 64 provide exemplary predicted secondary structures of the direct repeats of the disclosure.

In some embodiments, the gRNAs of the disclosure include non-naturally occurring, engineered direct repeat sequences which can be incorporated into the engineered gRNAs of the disclosure.

ii. Spacer Sequences

gRNAs of the disclosure comprise spacer sequences, complementary to the target DNA. More specifically, the nucleotide sequence of the targeter-RNA that is complementary to a target nucleotide sequence (the DNA-targeting sequence or spacer sequence) of the target DNA can have a length at least about 12 nt. For example, the DNA-targeting sequence of the targeter-RNA that is complementary to a target sequence of the target DNA can have a length at least about 12 nt, at least about 15 nt, at least about 18 nt, at least about 19 nt, at least about 20 nt, at least about 25 nt, at least about 30 nt, at least about 35 nt or at least about 40 nt. For example, the DNA-targeting sequence of the targeter-RNA that is complementary to a target sequence of the target DNA can have a length of from about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. The nucleotide sequence (the DNA-targeting sequence) of the targeter-RNA that is complementary to a nucleotide sequence (target sequence) of the target DNA can have a length at least about 12 nt. In some embodiments, the DNA-targeting sequence of the targeter-RNA that is complementary to a target sequence of the target DNA is 20 nucleotides in length. In some embodiments, the DNA-targeting sequence of the targeter-RNA that is complementary to a target sequence of the target DNA is 19 nucleotides in length.

The percent complementarity between the spacer sequence of the targeter-RNA and the target sequence of the target DNA can be at least 60% (e.g., at least 65%, at least 70%, at least 75%, at least 80%, at least 85%, at least 90%, at least 95%, at least 97%, at least 98%, at least 99%, or 100%). In some embodiments, the percent complementarity between the DNA-targeting sequence of the targeter-RNA and the target sequence of the target DNA is 100% over the 1-25 contiguous 5′-most nucleotides of the target sequence of the complementary strand of the target DNA. In some embodiments, the percent complementarity between the DNA-targeting sequence of the targeter-RNA and the target sequence of the target DNA is at least 60% over about 1-25 contiguous nucleotides. In some embodiments, the percent complementarity between the DNA-targeting sequence of the targeter-RNA and the target sequence of the target DNA is 100% over the 1-25 contiguous 5′-most nucleotides of the target sequence of the complementary strand of the target DNA and as low as 0% over the remainder. In such a case, the DNA-targeting sequence can be considered to be 1-25 nucleotides in length.

In some embodiments the spacer sequence of a Type II gRNA of the disclosure is directed to a target sequence in a mammalian organism. In some embodiments the spacer sequence is directed to a target sequence in a non-mammalian organism.

In some embodiments, the spacer sequence of a Type II gRNA of the disclosure is directed to a target sequence which is a sequence of a human. In some embodiments, the target sequence is a sequence of a non-human primate.

In some embodiments the spacer sequence of a Type II gRNA of the disclosure is directed to a target sequence selected of a therapeutic target.

In some embodiments the spacer sequence of a Type II gRNA of the disclosure is directed to a target sequence selected of a diagnostic target—for example in such embodiments a labeled catalytically dead Type II endonuclease of the disclosure and a gRNA directed to a diagnostic target DNA is contacted with the target DNA, or a cell comprising the target DNA, or a sample comprising the target DNA.

iii. Activator-RNA

The activator-RNA of a Type II gRNA of the disclosure binds with its cognate Type II endonuclease of the disclosure. The activator-RNA can interchangeably be referred to as a tracrRNA. The gRNA guides the bound Type II endonuclease to a specific nucleotide sequence within target DNA via the above described targeter-RNA. The activator-RNA of a Type II gRNA comprises two stretches of nucleotides that are complementary to one another. Exemplary tracrRNAs are provided herein, and include SEQ ID NO: 114, 119, 124, and 129. FIGS. 55, 58, 61, and 64 provide exemplary predicted secondary structures of the tracrRNAs of the disclosure.

iv. Dual-molecule Type H gRNAs

In some embodiments, provided herein are dual molecule (two-molecule) gRNAs for the novel Type II endonucleases of the disclosure. Such gRNAs comprise two separate RNA molecules (activator RNA-tracRNA; and the targeting RNA-crRNA). Each of the two RNA molecules of a subject double-molecule gRNA comprises a stretch of nucleotides that are complementary to one another such that the complementary nucleotides of the two RNA molecules hybridize to form the double stranded RNA duplex of the gRNA.

A dual-molecule gRNA can be designed to allow for controlled (i.e., conditional) binding of a targeter-RNA with an activator-RNA. Because a dual-molecule gRNA is not functional unless both the activator-RNA and the targeter-RNA are bound in a functional complex with Type II endonuclease of the disclosure, a dual-molecule gRNA can be inducible (e.g., drug inducible) by rendering the binding between the activator-RNA and the targeter-RNA to be inducible. As one non-limiting example, RNA aptamers can be used to regulate (i.e., control) the binding of the activator-RNA with the targeter-RNA. Accordingly, the activator-RNA and/or the targeter-RNA can comprise an RNA aptamer sequence.

The dual-molecule guide can be modified to include an aptamer

v. Single-Molecule Type II Endonuclease gRNAs

In some embodiments, provided herein are Type II gRNAs that comprises a single-molecule gRNA (interchangeably referred to herein as a sgRNA), for the novel Type II endonucleases of the disclosure.

Accordingly provided herein is an engineered single-molecule gRNA, comprising:
a. a targeter-RNA that is capable of hybridizing with a target sequence in a target DNA; and
b. an activator-RNA that is capable of hybridizing with the targeter-RNA to form a double-stranded RNA duplex, the activator-RNA comprising a activator-RNA,
   wherein the targeter-RNA and the activator-RNA are covalently linked to one another, wherein the single-molecule gRNA is capable of forming a complex with a novel Type II endonuclease of the disclosure, and wherein hybridization of the targeter-RNA to the target sequence is capable of targeting the Type II endonuclease of the disclosure to the target DNA.

A subject single-molecule gRNA comprises two segments of nucleotides (a targeter-RNA and an activator-RNA) that are complementary to one another, can be covalently linked by intervening nucleotides (“linkers” or “linker nucleotides”), and hybridize to form the double stranded RNA duplex (dsRNA duplex) of the activator-RNA, whereby resulting in a stem-loop structure. In some embodiments, the targeter-RNA and the activator-RNA are covalently linked via the 3′ end of the targeter-RNA and the 5′ end of the activator-RNA. In other embodiments, the activator-RNA is covalently linked via the 5′ end of the targeter-RNA and the 3′ end of the activator-RNA.

In some embodiments, the targeter-RNA and the activator-RNA are arranged in a 5′ to 3′ orientation.

In some embodiments, the activator-RNA and the targeter-RNA are arranged in a 5′ to 3′ orientation.

In some embodiments, the single molecule gRNA comprises one or more sequence modifications compared to a sequence of a corresponding wild type tracrRNA and/or crRNA.

In some embodiments, the targeter-RNA and the activator-RNA are covalently linked to one another via a linker.

When present, the linker of a single-molecule gRNA can have a length of from about 3 nucleotides to about 30 nucleotides. In exemplary embodiments, the linker of a single-molecule gRNA is 4, 5, 6, or 7 nt.

An exemplary single-molecule gRNA comprises two complementary stretches of nucleotides that hybridize to form a dsRNA duplex. In some embodiments, one of the two complementary stretches of nucleotides of the single-molecule gRNA (or the DNA encoding the stretch) is at least about 60% identical to one of the activator-RNA. For example, one of the two complementary stretches of nucleotides of the single-molecule gRNA (or the DNA encoding the stretch) is at least about 65% identical, at least about 70% identical, at least about 75% identical, at least about 80% identical, at least about 85% identical, at least about 90% identical, at least about 95% identical, at least about 98% identical, at least about 99% identical or 100% identical to an activator-RNA.

The activator-RNA and targeter-RNA segments can be engineered, while ensuring that the structure of the protein-binding domain of the gRNA is conserved. Thus, RNA folding structure of a naturally occurring protein-binding domain of a DNA-targeting RNA can be taken into account in order to design artificial protein-binding domains (either dual-molecule or single-molecule versions).

The activator-RNA in a single-molecule gRNA can have a length of from about 10 nucleotides to about 100 nucleotides. For example, the activator-RNA can have a length of from about 15 nucleotides (nt) to about 80 nt, from about 15 nt to about 50 nt, from about 15 nt to about 40 nt, from about 15 nt to about 30 nt or from about 15 nt to about 25 nt.

Also with regard to both the single-molecule and double-molecule gRNAs of the disclosure, the dsRNA duplex of the activator-RNA can have a length from about 6 nucleotides (nt) to about 50 bp. For example, the dsRNA duplex of the activator-RNA can have a length from about 6 nt to about 40 nt, from about 6 nt to about 30 bp, from about 6 nt to about 25 nt, from about 6 nt to about 20 nt, from about 6 nt to about 15 nt, from about 8 nt to about 40 nt, from about 8 nt to about 30 bp, from about 8 nt to about 25 nt, from about 8 nt to about 20 nt or from about 8 nt to about 15 nt. For example, the dsRNA duplex of the activator-RNA can have a length from about from about 8 nt to about 10 nt, from about 10 nt to about 15 nt, from about 15 nt to about 18 nt, from about 18 nt to about 20 nt, from about 20 nt to about 25 nt, from about 25 nt to about 30 nt, from about 30 nt to about 35 nt, from about 35 nt to about 40 nt, or from about 40 nt to about 50 nt. In some embodiments, the dsRNA duplex of the activator-RNA has a length of 8-15 base pairs. The percent complementarity between the nucleotide sequences that hybridize to form the dsRNA duplex of the activator-RNA can be at least about 60%. For example, the percent complementarity between the nucleotide sequences that hybridize to form the dsRNA duplex of the activator-RNA can be at least about 65%, at least about 70%, at least about 75%, at least about 80%, at least about 85%, at least about 90%, at least about 95%, at least about 98%, or at least about 99%. In some embodiments, the percent complementarity between the nucleotide sequences that hybridize to form the dsRNA duplex of the activator-RNA is 100%.

In some embodiments, the spacer sequence of a Type II gRNA (whether it is a single molecule gRNA or a dual molecule gRNA) of the disclosure is directed to a target sequence in a mammalian organism, e.g. a human or non-human primate. In some embodiments, the spacer sequence of a Type II gRNA of the disclosure is directed to a target sequence in a bacteria.

In some embodiments, the spacer sequence of a Type II gRNA of the disclosure is directed to a target sequence in a virus. In some embodiments, the spacer sequence of a Type II gRNA of the disclosure is directed to a target sequence in a plant.

In some embodiments, the single-molecule Type II gRNAs of the disclosure can be modified to include an aptamer.

vi. gRNA Arrays

The Type II gRNAs of the disclosure can be provided as gRNA arrays.

gRNA arrays include more than one gRNA arrayed in tandem, and can be processed into into two or more individual gRNAs. Thus, in some embodiments a precursor Type II gRNA array comprises two or more (e.g., 3 or more, 4 or more, 5 or more, 2, 3, 4, or 5) gRNAs (e.g., arrayed in tandem as precursor molecules). In some embodiments, two or more gRNAs can be present on an array (a precursor gRNA array). A Type II endonuclease of the disclosure can cleave the precursor gRNA array into individual gRNAs.

In some embodiments a gRNA array includes 2 or more gRNAs (e.g., 3 or more, 4 or more, 5 or more, 6 or more, or 7 or more, gRNAs). The gRNAs of a given array can target (i.e., can include guide sequences that hybridize to) different target sites of the same target DNA. In some embodiments, two or more gRNAs of a precursor gRNA array have the same guide sequence. In some embodiments, the precursor gRNA array comprises two or more gRNAs that target different target sites within the same target DNA. In some embodiments, the precursor gRNA array comprises two or more gRNAs that target different target DNAs.

### IV. Methods of Use—Modification and Therapeutics

a. Type II and Type V Endonuclease-Mediated Modification of Target DNA

Provided herein are uses of the novel Type II and Type V endonucleases of the disclosure, for the modification of a target DNA. In some embodiments the method of modifying a target DNA, the method comprising contacting the target DNA with any one of the Type II or Type V systems described herein.

In some embodiments, the target DNA is part of a chromosome in vitro. In some embodiments, the target DNA is part of a chromosome in vivo.

In some embodiments, the target DNA is part of a chromosome in a cell.

In some embodiments, the target DNA is extrachromosomal DNA.

In some embodiments, the target DNA is in a cell, wherein the cell is selected from the group consisting of: an archaeal cell, a bacterial cell, a eukaryotic cell, a eukaryotic single-cell organism, a somatic cell, a germ cell, a stem cell, a plant cell, an algal cell, an animal cell, in invertebrate cell, a vertebrate cell, a fish cell, a frog cell, a bird cell, a mammalian cell, a pig cell, a cow cell, a goat cell, a sheep cell, a rodent cell, a rat cell, a mouse cell, a non-human primate cell, and a human cell.

In some embodiments, the target DNA is the DNA of a parasite.

In some embodiments, the target DNA is a viral DNA.

In some embodiments, the target DNA is a bacterial DNA.

In some embodiments, the modifying comprises introducing a double strand break in the target DNA.

In some embodiments, the contacting occurs under conditions that are permissive for non-homologous end joining or homology-directed repair.

In some embodiments, the method comprises contacting the target DNA with a donor polynucleotide, wherein the donor polynucleotide, a portion of the donor polynucleotide, a copy of the donor polynucleotide, or a portion of a copy of the donor polynucleotide integrates into the target DNA.

In some embodiments, the method does not comprise contacting the cell with a donor polynucleotide, wherein the target DNA is modified such that nucleotides within the target DNA are deleted.

b. Type VI Endonuclease-Mediated Modification of Target RNA

Provided herein are uses of the novel Type VI endonucleases of the disclosure, for the modification of a target RNA. In some embodiments the method of modifying a target RNA, the method comprising contacting the target RNA with any one of the Type VI systems described herein.

In some embodiments, the target RNA is in vitro. In some embodiments, the target RNA in vivo.

In some embodiments, the target RNA is in a cell, wherein the cell is selected from the group consisting of: an archaeal cell, a bacterial cell, a eukaryotic cell, a eukaryotic single-cell organism, a somatic cell, a germ cell, a stem cell, a plant cell, an algal cell, an animal cell, in invertebrate cell, a vertebrate cell, a fish cell, a frog cell, a bird cell, a mammalian cell, a pig cell, a cow cell, a goat cell, a sheep cell, a rodent cell, a rat cell, a mouse cell, a non-human primate cell, and a human cell.

In some embodiments, the target RNA is the RNA of a parasite.

In some embodiments, the target RNA is a viral RNA.

In some embodiments, the target RNA is a bacterial RNA.

The target RNA may be any suitable form of RNA. This may include, in some embodiments, mRNA. In other embodiments, the target RNA may include tRNA or rRNA. In other embodiments, the target RNA may include miRNA. In other embodiments, the target RNA may include siRNA.

c. Therapeutic Applications (Type II, Type V endonucleases)

The disclosure provides novel Type II, and Type V endonucleases, engineered systems, one or more polynucleotides encoding components of said system, and vector or delivery systems comprising one or more polynucleotides encoding components of said system for use in therapeutic methods. The therapeutic methods may comprise gene or genome editing, or gene therapy. The therapeutic methods comprise use and delivery of the novel Type II or Type V endonucleases of the disclosure.

Accordingly, in some embodiments, provided herein is a method of modifying a target DNA, the method comprising contacting a target DNA, a cell comprising the target DNA, or a subject with cells with the target DNA, with any one of the Type II and Type V systems described herein. In other embodiments, provided herein is a method of modifying a target RNA, the method comprising contacting a target RNA, a cell comprising the target RNA, or a subject with cells with the target RNA, with any one of the Type VI systems described herein.

In some embodiments, the target DNA is part of a chromosome in vitro. In some embodiments, the target DNA is part of a chromosome in vivo.

In some embodiments, the target DNA is part of a chromosome in a cell.

In some embodiments, the target DNA is extrachromosomal DNA.

In some embodiments, the target DNA is in a cell, wherein the cell is selected from the group consisting of: an archaeal cell, a bacterial cell, a eukaryotic cell, a eukaryotic single-cell organism, a somatic cell, a germ cell, a stem cell, a plant cell, an algal cell, an animal cell, in invertebrate cell, a vertebrate cell, a fish cell, a frog cell, a bird cell, a mammalian cell, a pig cell, a cow cell, a goat cell, a sheep cell, a rodent cell, a rat cell, a mouse cell, a non-human primate cell, and a human cell.

In some embodiments, the target DNA is outside of a cell.

In some embodiments, the target DNA is in vitro inside of a cell.

In some embodiments, the target DNA is in vivo, inside of a cell.

In some embodiments, the modifying comprises introducing a double strand break in the target DNA.

In some embodiments, the contacting occurs under conditions that are permissive for non-homologous end joining or homology-directed repair.

In some embodiments, the method comprises contacting the target DNA with a donor polynucleotide, wherein the donor polynucleotide, a portion of the donor polynucleotide, a copy of the donor polynucleotide, or a portion of a copy of the donor polynucleotide integrates into the target DNA.

In some embodiments, the method does not comprise contacting the cell with a donor polynucleotide, wherein the target DNA is modified such that nucleotides within the target DNA are deleted.

In some embodiments, the therapeutic methods involve modifying a target DNA comprising a target sequence of a gene of interest and/or the regulatory region of the gene of interest, the method comprising delivering to a cell comprising the target DNA, a Type II endonuclease of the disclosure and one or more Type II gRNAs, a Type V endonuclease of the disclosure and one or more Type V gRNAs, one or more nucleotides encoding the Type II endonuclease of the disclosure and one or more Type II gRNAs, or one or more nucleotides encoding a Type V endonuclease of the disclosure and one or more Type V gRNAs.

In some embodiments, the gene of interest is within a eukaryotic cell, e.g. a human or non-human primate cell.

In some embodiments, the gene of interest is within a plant cell.

In some embodiments, the delivering comprises delivering to the cell a Type II endonuclease of the disclosure (or one or more nucleotides encoding the same) and one or more Type II gRNAs.

In some embodiments, the delivering comprises delivering to the cell a Type V endonuclease of the disclosure (or one or more nucleotides encoding the same) and one or more Type V gRNAs.

In some embodiments, the delivering comprises delivering to the cell one or more nucleotides encoding the Type II endonuclease of the disclosure and one or more Type II gRNAs.

In some embodiments, the delivering comprises delivering to the cell one or more nucleotides encoding a Type V endonuclease of the disclosure and one or more Type V gRNAs.

d. Therapeutic Applications (Type VI Endonucleases)

The disclosure provides novel Type VI endonucleases, engineered systems, one or more polynucleotides encoding components of said system, and vector or delivery systems comprising one or more polynucleotides encoding components of said system for use in therapeutic methods.

Accordingly, in some embodiments, provided herein is a method of modifying a target RNA, the method comprising contacting a target RNA, a cell comprising the target RNA, or a subject with cells with the target RNA, with any one of the Type VI systems described herein. In other embodiments, provided herein is a method of modifying a target RNA, the method comprising contacting a target RNA, a cell comprising the target RNA, or a subject with cells with the target RNA, with any one of the Type VI systems described herein.

In some embodiments, the target RNA is in a cell, wherein the cell is selected from the group consisting of: an archaeal cell, a bacterial cell, a eukaryotic cell, a eukaryotic single-cell organism, a somatic cell, a germ cell, a stem cell, a plant cell, an algal cell, an animal cell, in invertebrate cell, a vertebrate cell, a fish cell, a frog cell, a bird cell, a mammalian cell, a pig cell, a cow cell, a goat cell, a sheep cell, a rodent cell, a rat cell, a mouse cell, a non-human primate cell, and a human cell.

In some embodiments, the target RNA is outside of a cell.

In some embodiments, the target RNA is in vitro inside of a cell.

In some embodiments, the target RNA is in vivo, inside of a cell.

The target RNA may be any suitable form of RNA. This may include, in some embodiments, mRNA. In other embodiments, the target RNA may include tRNA or rRNA. In other embodiments, the target RNA may include miRNA. In other embodiments, the target RNA may include siRNA.

In some embodiments, the therapeutic methods involve modifying a target RNA comprising a mRNA encoding a gene of interest and/or the regulatory region of the mRNA of interest, the method comprising delivering to a cell comprising the target RNA, a Type VI endonuclease of the disclosure and one or more Type VI gRNAs, or one or more nucleotides encoding the Type VI endonuclease of the disclosure and one or more Type VI gRNAs.

In some embodiments, the RNA of interest is within a eukaryotic cell, e.g. a human or non-human primate cell.

In some embodiments, the RNA of interest is within a plant cell.

In some embodiments, the delivering comprises delivering to the cell a Type VI endonuclease of the disclosure (or one or more nucleotides encoding the same) and one or more Type VI gRNAs.

In some embodiments, the delivering comprises delivering to the cell one or more nucleotides encoding a Type VI endonuclease of the disclosure and one or more Type VI gRNAs.

e. Delivery

Delivery of the Type II, Type V, and Type VI components to a cell can be achieved by any variety of delivery methods known to those of skill in the art. As a non-limiting example, the components can be combined with a lipid. As another non-limiting example, the components combined with a particle, or formulated into a particle, e.g. a nanoparticle.

Methods of introducing a nucleic acid and/or protein into a host cell are known in the art, and any convenient method can be used to introduce a subject nucleic acid (e.g., an expression construct/vector) into a target cell (e.g., prokaryotic cell, eukaryotic cell, plant cell, animal cell, mammalian cell, human cell, and the like). Suitable methods include, e.g., viral infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery and the like.

A gRNA can be introduced, e.g., as a DNA molecule encoding the gRNA, or can be provided directly as an RNA molecule (or a chimeric/hybrid molecule when applicable).

In some embodiments, Type II, Type V, or Type VI endonuclease is provided as a nucleic acid (e.g., an mRNA, a DNA, a plasmid, an expression vector, a viral vector, etc.) that encodes the protein.

In some embodiments, the Type II, Type V, or Type VI endonuclease is provided directly as a protein (e.g., without an associated gRNA or with an associate gRNA, i.e., as a ribonucleoprotein complex—RNP). Like a gRNA, a Type II, Type V, or Type VI endonuclease of the disclosure can be introduced into a cell (provided to the cell) by any convenient method; such methods are known to those of ordinary skill in the art. As an illustrative example, a Type II, Type V, or Type VI endonuclease of the disclosure can be injected directly into a cell (e.g., with or without a gRNA or nucleic acid encoding a gRNA). As another example, a pre-formed complex of a Type II, Type V, or Type VI endonuclease and a gRNA can be introduced into a cell (e.g., eukaryotic cell) (e.g., via injection, via nucleofection; via a protein transduction domain (PTD) conjugated to one or more components, e.g., conjugated to the Type II, Type V, or Type VI endonuclease of the disclosure, conjugated to a gRNA; etc.).

In some embodiments, a nucleic acid (e.g., a gRNA; a nucleic acid comprising a nucleotide sequence encoding a Type II, Type V, or Type VI endonuclease of the disclosure; etc.) and/or a polypeptide (e.g., a Type II, Type V, or Type VI endonuclease of the disclosure) is delivered to a cell (e.g., a target host cell) in a particle, or associated with a particle. In some embodiments, the particle is a nanoparticle.

A Type II, Type V, or Type VI endonuclease of the disclosure (or an mRNA comprising a nucleotide sequence encoding the protein) and/or gRNA (or a nucleic acid such as one or more expression vectors encoding the gRNA) may be delivered simultaneously using particles or lipid envelopes.

f. Target Cells of Interest

Suitable target cells (which can comprise target DNA such as genomic DNA or target RNA) include, but are not limited to: a bacterial cell; an archaeal cell; a cell of a single-cell eukaryotic organism; a plant cell; an algal cell, e.g., Botryococcus braunii, Chlamydomonas reinhardtii, Nannochloropsis gaditana, Chlorella pyrenoidosa, Sargassum patens, C. agardh, and the like; a fungal cell (e.g., a yeast cell); an animal cell; a cell from an invertebrate animal (e.g. fruit fly, a cnidarian, an echinoderm, a nematode, etc.); a cell of an insect (e.g., a mosquito; a bee; an agricultural pest; etc.); a cell of an arachnid (e.g., a spider; a tick; etc.); a cell from a vertebrate animal (e.g., a fish, an amphibian, a reptile, a bird, a mammal); a cell from a mammal (e.g., a cell from a rodent; a cell from a human; a cell of a non-human mammal; a cell of a rodent (e.g., a mouse, a rat); a cell of a lagomorph (e.g., a rabbit); a cell of an ungulate (e.g., a cow, a horse, a camel, a llama, a vicuna, a sheep, a goat, etc.); a cell of a marine mammal (e.g., a whale, a seal, an elephant seal, a dolphin, a sea lion; etc.) and the like.

Any type of cell may be of interest (e.g. a stem cell, e.g. an embryonic stem (ES) cell, an induced pluripotent stem cell (iPSC), a germ cell (e.g., an oocyte, a sperm, an oogonia, a spermatogonia, etc.), an adult stem cell, a somatic cell, e.g. a fibroblast, a hematopoietic cell, a neuron, a muscle cell, a bone cell, a hepatocyte, a pancreatic cell; an in vitro or in vivo embryonic cell of an embryo at any stage, e.g., a 1-cell, 2-cell, 4-cell, 8-cell, etc. stage zebrafish embryo; etc.).

Cells may be from cell lines or primary cells. Target cells can be unicellular organisms and/or can be grown in culture. If the cells are primary cells, they may be harvest from an individual by any convenient method. For example, leukocytes may be conveniently harvested by apheresis, leukocytapheresis, density gradient separation, etc., while cells from tissues such as skin, muscle, bone marrow, spleen, liver, pancreas, lung, intestine, stomach, etc. can be conveniently harvested by biopsy.

Because the gRNA provides specificity by hybridizing to target nucleic acid, a mitotic and/or post-mitotic cell of interest in the disclosed methods may include a cell of any organism (e.g. a bacterial cell, an archaeal cell, a cell of a single-cell eukaryotic organism, a plant cell, an algal cell, e.g., Botryococcus braunii, Chlamydomonas reinhardtii, Nannochloropsis gaditana, Chlorella pyrenoidosa, Sargassum patens, C. agardh, and the like, a fungal cell (e.g., a yeast cell), an animal cell, a cell of an invertebrate animal (e.g. fruit fly, cnidarian, echinoderm, nematode, etc.), a cell of a vertebrate animal (e.g., fish, amphibian, reptile, bird, mammal), a cell of a mammal, a cell of a rodent, a cell of a human, etc.).

Plant cells include cells of a monocotyledon, and cells of a dicotyledon. The cells can be root cells, leaf cells, cells of the xylem, cells of the phloem, cells of the cambium, apical meristem cells, parenchyma cells, collenchyma cells, sclerenchyma cells, and the like. Plant cells include cells of agricultural crops such as wheat, corn, rice, sorghum, millet, soybean, etc. Plant cells include cells of agricultural fruit and nut plants, e.g., plant that produce apricots, oranges, lemons, apples, plums, pears, almonds, etc.

Non-limiting examples of cells (target cells) include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatos, rice, cassava, sugarcane, pumpkin, hay, potatos, cotton, cannabis, tobacco, flowering plants, conifers, gymnosperms, angiosperms, ferns, clubmosses, hornworts, liverworts, mosses, dicotyledons, monocotyledons, etc.), an algal cell, (e.g., Botryococcus braunii, Chlamydomonas reinhardtii, Nannochloropsis gaditana, Chlorella pyrenoidosa, Sargassum patens, C. agardh, and the like), seaweeds (e.g. kelp) a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., an ungulate (e.g., a pig, a cow, a goat, a sheep); a rodent (e.g., a rat, a mouse); a non-human primate; a human; a feline (e.g., a cat); a canine (e.g., a dog); etc.), and the like. In some embodiments, the cell is a cell that does not originate from a natural organism (e.g., the cell can be a synthetically made cell; also referred to as an artificial cell).

A cell can be an in vitro cell (e.g., established cultured cell line). A cell can be an ex vivo cell (cultured cell from an individual). A cell can be and in vivo cell (e.g., a cell in an individual). A cell can be an isolated cell. A cell can be a cell inside of an organism. A cell can be an organism.

Suitable cells include human embryonic stem cells, fetal cardiomyocytes, myofibroblasts, mesenchymal stem cells, autotransplated expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

In some embodiments, the cell is an immune cell, a neuron, an epithelial cell, and endothelial cell, or a stem cell. In some embodiments, the immune cell is a T cell, a B cell, a monocyte, a natural killer cell, a dendritic cell, or a macrophage. In some embodiments, the immune cell is a cytotoxic T cell. In some embodiments, the immune cell is a helper T cell. In some embodiments, the immune cell is a regulatory T cell (Treg).

In some embodiments, the cell is a stem cell. Stem cells include adult stem cells. Adult stem cells are also referred to as somatic stem cells.

Adult stem cells are resident in differentiated tissue, but retain the properties of self-renewal and ability to give rise to multiple cell types, usually cell types typical of the tissue in which the stem cells are found. Numerous examples of somatic stem cells are known to those of skill in the art, including muscle stem cells; hematopoietic stem cells; epithelial stem cells; neural stem cells; mesenchymal stem cells; mammary stem cells; intestinal stem cells; mesodermal stem cells; endothelial stem cells; olfactory stem cells; neural crest stem cells; and the like.

Stem cells of interest include mammalian stem cells, where the term “mammalian” refers to any animal classified as a mammal, including humans; non-human primates; domestic and farm animals; and zoo, laboratory, sports, or pet animals, such as dogs, horses, cats, cows, mice, rats, rabbits, etc. In some embodiments, the stem cell is a human stem cell. In some embodiments, the stem cell is a rodent (e.g., a mouse; a rat) stem cell. In some embodiments, the stem cell is a non-human primate stem cell.

g. Targets

Any gene of interest can serve as a target for modification.

In particular embodiments, the target is a gene or mRNA implicated in cancer. In particular embodiments, the target is a gene or mRNA implicated in an immune disease, e.g. an autoimmune disease. In particular embodiments, the target is a gene or mRNA implicated in a neurodegenerative disease. In particular embodiments, the target is a gene or mRNA implicated in a neuropsychiatric disease. In particular embodiments, the target is a gene or mRNA implicated in a muscular disease. In particular embodiments, the target is a gene or mRNA implicated in a cardiac disease. In particular embodiments, the target is a gene implicated in diabetes. In particular embodiments, the target is a gene implicated in kidney disease.

h. Precursor gRNA Arrays

The therapeutic methods provided herein can include delivery of precursor gRNA arrays. A Type II, Type V, or Type VI endonuclease of the disclosure can cleave a precursor gRNA into a mature gRNA, e.g., by endoribonucleolytic cleavage of the precursor. A Type II, Type V, or Type VI endonuclease of the disclosure can cleave a precursor gRNA array (that includes more than one gRNA arrayed in tandem) into two or more individual gRNAs.

### V. Methods of Use—Detection and Diagnostic Applications

In addition to the ability to cleave a target sequence in a targeted DNA, the Type V or Type VI endonucleases of the disclosure also possess collateral (trans-cleavage activity), i.e. the ability to promiscuously cleave non-targeted oligonucleotides, once activated by detection of a target DNA or RNA. Without being bound to any theory or mechanism, generally once a Type V or Type VI endonuclease of the disclosure is activated by a gRNA, which occurs when a sample includes a target sequence to which the gRNA hybridizes (i.e., the sample includes the targeted DNA or the targeted RNA), the Type V or Type VI becomes a nuclease that promiscuously cleaves single stranded oligonucleotides (i.e., non-target single stranded oligonucleotides, i.e., single stranded oligonucleotides to which the guide sequence of the gRNA does not hybridize). Thus, when the targeted DNA (double or single stranded) or RNA is present in the sample (e.g., in some embodiments above a threshold amount), the result can be cleavage (collateral) of oligonucleotides in the sample, which can be detected using any convenient detection method (e.g., using a labeled single stranded detector DNA, labeled detector RNA, or labeled detector DNA/RNA chimeric oligonucleotides).

Accordingly, provided herein are methods and compositions for detecting a target DNA (dsDNA or ssDNA) or RNA in a sample. Also provided are methods and compositions for cleaving non-target oligonucleotides (e.g. used as detectors).

As used herein, generally a “detector” comprises an oligonucleotide of any nature, single or double stranded and does not hybridize with the guide sequence of the gRNA (i.e., the detector oligonucleotide that is a non-target). Exemplary detectors include, but are not limited to ssDNA, dsDNA, ssRNA, ss DNA/RNA chimeras, dsRNA, RNA comprising ss and ds regions, and ss or ds oligonucleotides containing RNA and DNA nucleotides (as used herein ss=single stranded; and ds=double stranded). Ultimately, the preference of the particular CRISPR-Cas protein in question will be determined, and the appropriate detector(s) will be utilized.

The detection methods based on the collateral activity of the Type V or Type VI endonucleases of the disclosure can include:

(a) contacting the sample with: (i) a Type V or Type VI endonuclease of the disclosure; (ii) a gRNA comprising: a region that binds to the Type V or Type VI endonuclease, and a guide sequence that hybridizes with the target DNA; and (iii) a detector that does not hybridize with the guide sequence of the gRNA; and

(b) measuring a detectable signal produced by cleavage of the detector by the Type V or Type VI endonuclease, thereby detecting the target DNA.

Once a subject Type V or Type VI endonuclease is activated by a gRNA, which can occur when the sample includes a target DNA to which the gRNA hybridizes (i.e., the sample includes the targeted sequence in the target DNA), the Type V or Type VI can be activated to function as an endoribonuclease that non-specifically cleaves detector oligonucleotides (including non-target ss oligonucleotides) present in the sample. Thus, when the target DNA is present in the sample, the result is cleavage of a detector oligonucleotide in the sample, which can be detected using any convenient detection method (e.g., using a labeled detector oligonucleotides).

Also provided are methods and compositions for cleaving detector oligonucleotides (e.g., ssDNAs, ssRNAs, ssDNA/RNA chimeras or detectors comprising ss and ds regions). Such methods can include contacting a population of nucleic acids, wherein said population comprises a target DNA and a plurality of non-target ss oligonucleotides, with: (i) a Type V or Type VI endonuclease of the disclosure; and (ii) a gRNA comprising: a region that binds to the Type V or Type VI effector protein, and a guide sequence that hybridizes with the target DNA, wherein the Type V or Type VI endonuclease cleaves non-target ss oligonucleotides

Accordingly, provided herein is a method of detecting a target DNA or RNA in a sample, the method comprising:

(a) contacting the sample with:

(i) a Type V or Type VI endonuclease of the disclosure;

(ii) a gRNA comprising a spacer sequence that is capable of hybridizing with a target sequence in a target DNA or RNA; and

(iii) a labeled detector oligonucleotide that does not hybridize with the spacer sequence of the gRNA; and

(b) measuring a detectable signal produced by cleavage of the labeled detector oligonucleotide by the Type V or Type VI endonuclease, thereby detecting the target target DNA or RNA.

In some embodiments, the contacting step can be carried out in an acellular environment, e.g., outside of a cell. In other embodiments, contacting step can be carried out inside a cell. The contacting step can be carried out in a cell in vitro. The contacting step can be carried out in a cell in vivo. The contacting step of a detection method can be carried out in a composition comprising divalent metal ions.

The gRNA can be provided as RNA or as a nucleic acid encoding the gRNA (e.g., a DNA such as a recombinant expression vector), described herein.

The contacting, prior to the measuring step, can last for any period of time, e.g from 5 seconds to 2 hours or more, prior to the measuring step. In some embodiments the sample is contacted for 45 minutes or less prior to the measuring step. In some embodiments the sample is contacted for 30 minutes or less prior to the measuring step. In some embodiments the sample is contacted for 10 minutes or less prior to the measuring step. In some embodiments the sample is contacted for 5 minutes or less prior to the measuring step. In some embodiments the sample is contacted for 1 minute or less prior to the measuring step. In some embodiments the sample is contacted for from 50 seconds to 60 seconds prior to the measuring step. In some embodiments the sample is contacted for from 40 seconds to 50 seconds prior to the measuring step. In some embodiments the sample is contacted for from 30 seconds to 40 seconds prior to the measuring step. In some embodiments the sample is contacted for from 20 seconds to 30 seconds prior to the measuring step. In some embodiments the sample is contacted for from 10 seconds to 20 seconds prior to the measuring step.

The detection methods provided herein can detect a target DNA or RNA with a high degree of sensitivity. Accordingly, in some embodiments, the detection methods of the disclosure can be used to detect a target DNA or RNA present in a sample comprising a plurality of DNA or RNA (including the target DNA or RNA and a plurality of non-target DNAs or RNAs), where the target DNA or RNA is present at one or more copies per 5 to 10\hat{ }9 copies of the non-target DNAs or RNAs).

In some embodiments, the threshold of detection, for a detection method of detecting a target DNA or RNA in a sample, is 10 nM or less. The term “threshold of detection” is used herein to describe the minimal amount of target DNA or RNA that must be present in a sample in order for detection to occur. In some embodiments, a subject composition or method exhibits an attomolar (aM) sensitivity of detection. In some embodiments, a subject composition or method exhibits a femtomolar (fM) sensitivity of detection. In some embodiments, a subject composition or method exhibits a picomolar (pM) sensitivity of detection. In some embodiments, a subject composition or method exhibits a nanomolar (nM) sensitivity of detection.

a. Target DNA and RNA

A target DNA can be single stranded (ssDNA) or double stranded (dsDNA). There need not be any preference or requirement for a PAM sequence in a single stranded target DNA. A target RNA can be single stranded RNA.

The source of the target DNA or RNA can be any source. In some embodiments the target DNA or RNA is a viral or bacterial DNA or RNA (e.g., a genomic DNA or RNA of a DNA or RNA virus or bacteria). As such, detection method can be for detecting the presence of a viral or bacterial DNA amongst a population of nucleic acids (e.g., in a sample). In the case of a RNA-carrying organism, for example, a RNA virus (e.g. a coronavirus)—it is understood that a step such as reverse transcription may be carried out on a sample comprising the RNA-carrying organism to generated cDNA, and the cDNA is then the target DNA. Alternatively, the RNA can also be detected directly using a Type VI endonuclease of the disclosure.

Exemplary non-limiting sources for target DNA or RNA are provided in Tables 10a-10f. Without being limited to a particular methodology, if the genome of the target is a DNA, and the CRISPR-Cas enzyme utilized is an RNA-targeting enzyme, an in vitro transcription (IVT) step could be included to transcribe the genome to RNA, prior to assessment. Likewise, without being limited to a particular methodology, if the genome of the target is a RNA, and the CRISPR-Cas enzyme utilized is an DNA-targeting enzyme, a reverse transcriptase (RT) step could be included to reverse transcribe the genome to DNA, prior to assessment.

TABLE 10a
Bacterial Resistance Gene Targets

KPC: carbapenem-hydrolyzing class A beta-lactamase
NDM: metallo-beta-lactamase
OXA: oxacillin-hydrolyzing class D beta-lactamase
MecA: PBP2a family beta-lactam-resistant peptidoglycan transpeptidase
vanA/B: Vancomycin resistance

TABLE 10b
Virus Genome Targets

Dengue (DENY) fever virus (subtypes 1,2, 3 and 4)
Zika Virus
Chikungunya virus
Coronoavirus

### Respiratory Targets

DNA or RNA obtained from viruses and bacteria related to respiratory infections may also be targeted. A list of targets of interest may include the examples shown in Table 10c.

TABLE 10c

Respiratory Targets
Adenovirus
Coronoavirus
SARS-CoV
SARS-CoV-2
MERS-CoV
Coronavirus HKU1
Coronavirus NL63
Coronavirus 229E
Coronavirus OC43
Coronovirus HKU1
Human Metapneumovirus
Human Rhinovirus/Enterovirus
Influenza A
Influenza A/H1
Influenza A/H3
Influenza A/H1-2009
Influenza B
Parainfluenza Virus 1
Parainfluenza Virus 2
Parainfluenza Virus 3
Parainfluenza Virus 4
Respiratory Syncytial Virus
BACTERIA:
Bordetella parapertussis
Bordetella pertussis
Chlamydia pneumoniae
Mycoplasma pneumoniae

### Sexually Transmitted Disease Targets

DNA or RNA obtained from viruses and bacteria related to sexually transmitted diseases may also be targeted. A list of targets of interest may include the examples shown in Table 10d.

TABLE 10d

Sexually Transmitted Disease Targets
HIV (Type 1 and type 2)
Herpes Simplex Virus 1 (HSV-1)
Herpes Simplex Virus 2 (HSV-2)
Hepatitis A
Hepatitis B
Hepatitis C
BACTERIA
Treponema pallidum
Chlamydia
Neisseria gonorrhoeae

### Other Targets

Other DNA or RNA targets may also be targeted. As another example, male genes to determine the sex of the embryo of a pregnant woman/animal, and the male genes to determine the sex of plants and seeds may also be targeted. Examples of further targets of interest may include the following shown in Table 10e.

TABLE 10e
Viral

Papovavirus (e.g., human papillomavirus (HPV), polyomavirus)
Hepadnavirus (e.g., Hepatitis B Virus (HBV))
Herpesvirus (e.g., herpes simplex virus (HSV)
Varicella zoster virus (VZV)
Epstein-barr virus (EBV)
Cytomegalovirus (CMV)
Herpes lymphotropic virus, Pityriasis Rosea, kaposi's sarcoma-associated herpesvirus);
Adenovirus (e.g., atadenovirus, aviadenovirus, ichtadenovirus, mastadenovirus, siadenovirus)
Poxvirus (e.g., smallpox, vaccinia virus, cowpox virus, monkeypox virus, orf virus,
pseudocowpox, bovine papular stomatitis virus; tanapox virus, yaba monkey tumor virus;
molluscum contagiosum virus (MCV))
Parvovirus (e.g., adeno-associated virus (AAV), Parvovirus B19, human bocavirus, bufavirus,
human parv4 G1); Geminiviridae; Nanoviridae; Phycodnaviridae; and the like.
Dengue fever virus (subtypes 1, 2, 3, and 4)
Zika virus
Hantavirus
Chikungunya virus

Other miscellaneous targets of interest that provide sources for DNA or RNA targets are shown in Table 10f.

TABLE 10f
Sex determination targets

SRY genes of mammals and non-mammal animals
Other miscellaneous targets of interest
hHPRT1 (hypoxanthine phosphoribosyltransferase 1)
16S E. coli

b. Samples

The term “sample” is used herein to mean any sample that includes DNA or RNA (e.g., in order to determine whether a target DNA or RNA is present among a population of DNA or RNAs). As noted above, the DNA can be single stranded, double stranded DNA, complementary DNA, and the like.

A sample intended for detection comprises a plurality of nucleic acids. Thus, in some embodiments a sample includes two or more (e.g., 3 or more, 5 or more, 10 or more, 20 or more, 50 or more, 100 or more, 500 or more, 1,000 or more, or 5,000 or more) nucleic acids (e.g., DNA or RNAs). A detection method can be used as a very sensitive way to detect a target DNA or RNA present in a sample (e.g., in a complex mixture of nucleic acids such as DNA or RNAs).

In some embodiments the sample includes 5 or more DNA or RNAs (e.g., 10 or more, 20 or more, 50 or more, 100 or more, 500 or more, 1,000 or more, or 5,000 or more DNA or RNAs) that differ from one another in sequence. In some embodiments, the sample includes 10 or more, 20 or more, 50 or more, 100 or more, 500 or more, 10\hat{ }3 or more, 5×10\hat{ }3 or more, 10\hat{ }4 or more, 5×10\hat{ }4 or more, 10\hat{ }5 or more, 5×10\hat{ }5 or more, 10\hat{ }6 or more 5×10\hat{ }6 or more, or 10\hat{ }7 or more, DNA or RNAs. In some embodiments, the sample comprises from 10 to 20, from 20 to 50, from 50 to 100, from 100 to 500, from 500 to 10\hat{ }3, from 10\hat{ }3 to 5×10\hat{ }3, from 5×10\hat{ }3 to 10\hat{ }4, from 10\hat{ }4 to 5×10\hat{ }4, from 5×10\hat{ }4 to 10\hat{ }5, from 10\hat{ }5 to 5×10\hat{ }5, from 5×10\hat{ }5 to 10\hat{ }6, from 10\hat{ }6 to 5×10\hat{ }6, or from 5×10\hat{ }6 to 10\hat{ }7, or more than 10\hat{ }7, DNA or RNAs. In some embodiments, the sample comprises from 5 to 10\hat{ }7 DNA or RNAs (e.g., that differ from one another in sequence)(e.g., from 5 to 10\hat{ }6, from 5 to 10\hat{ }5, from 5 to 50,000, from 5 to 30,000, from 10 to 10\hat{ }6, from 10 to 10\hat{ }5, from 10 to 50,000, from 10 to 30,000, from 20 to 10\hat{ }6, from 20 to 10\hat{ }5, from 20 to 50,000, or from 20 to 30,000 DNA or RNAs).

In some embodiments the sample includes 20 or more DNA or RNAs that differ from one another in sequence. In some embodiments, the sample includes DNA or RNAs from a cell lysate (e.g., a eukaryotic cell lysate, a mammalian cell lysate, a human cell lysate, a prokaryotic cell lysate, a plant cell lysate, and the like). For example, in some embodiments the sample includes DNA or RNA from a cell such as a eukaryotic cell, e.g., a mammalian cell such as a human cell.

The sample can be derived from any source, e.g., the sample can be a synthetic combination of purified DNA or RNAs; the sample can be a cell lysate, a DNA or RNA-enriched cell lysate, or DNA or RNAs isolated and/or purified from a cell lysate. The sample can be from a patient (e.g., for the purpose of diagnosis). The sample can be from permeabilized cells. The sample can be from crosslinked cells. The sample can be in tissue sections.

A sample can include a target DNA or RNA and a plurality of non-target DNA or RNAs. In some embodiments, the target DNA or RNA is present in the sample at one or more copies per 5 to 10\hat{ }9 copies of the non-target DNA or RNAs.

Suitable samples include but are not limited to urine, blood, serum, plasma, lymphatic fluid, cerebrospinal fluid, saliva, nasopharyngeal, oropharyngeal, nasopharyngeal/oropharyngeal, aspirate, or biopsy sample. Thus, the term “sample” with respect to a patient encompasses blood and other liquid samples of biological origin, solid tissue samples such as a biopsy specimen or tissue cultures or cells derived therefrom and the progeny thereof. Samples also can be samples that have been manipulated in any way after their procurement, such as by treatment with reagents; washed; or enrichment for certain cell populations, such as cancer cells. The samples can be obtained by use of a swab, for example, a nasopharyngeal swab, an oropharyngeal swab, or a nasopharyngeal/oropharyngeal swab. Samples also can be samples that have been enriched for particular types of molecules, e.g., DNA or RNAs. Samples encompasses biological samples such as a clinical sample such as blood, plasma, serum, aspirate, cerebral spinal fluid (CSF), and also includes tissue obtained by surgical resection, tissue obtained by biopsy, cells in culture, cell supernatants, cell lysates, tissue samples, organs, bone marrow, and the like. A “biological sample” includes biological fluids derived therefrom (e.g., cancerous cell, infected cell, etc.), e.g., a sample comprising DNA or RNAs that is obtained from such cells (e.g., a cell lysate or other cell extract comprising DNA or RNAs).

A sample can comprise, or can be obtained from, any of a variety of cells, tissues, organs, or acellular fluids. Suitable sample sources include eukaryotic cells, bacterial cells, and archaeal cells. Suitable sample sources include single-celled organisms and multi-cellular organisms. Suitable sample sources include single-cell eukaryotic organisms; a plant or a plant cell; an algal cell; a fungal cell; an animal cell, tissue, or organ; a cell, tissue, or organ from an invertebrate animal; a cell, tissue, fluid, or organ from a vertebrate animal; a cell, tissue, fluid, or organ from a mammal (e.g., a human; a non-human primate; an ungulate; a feline; a bovine; an ovine; a caprine; etc.). Suitable sample sources include nematodes, protozoans, and the like. Suitable sample sources include parasites such as helminths, malarial parasites, etc.

Suitable sample sources include a cell, tissue, or organism of any of the six kingdoms.

Suitable sources of a sample include cells, fluid, tissue, or organ taken from an organism; from a particular cell or group of cells isolated from an organism; etc. For example, where the organism is a plant, suitable sources include xylem, the phloem, the cambium layer, leaves, roots, etc. Where the organism is an animal, suitable sources include particular tissues (e.g., lung, liver, heart, kidney, brain, spleen, skin, fetal tissue, etc.), or a particular cell type (e.g., neuronal cells, epithelial cells, endothelial cells, astrocytes, macrophages, glial cells, islet cells, T lymphocytes, B lymphocytes, etc.).

In some embodiments, the source of the sample is a (or is suspected of being a diseased cell, fluid, tissue, or organ.

In some embodiments, the source of the sample is a normal (non-diseased) cell, fluid, tissue, or organ.

In some embodiments, the source of the sample is a (or is suspected of being a pathogen-infected cell, tissue, or organ. For example, the source of a sample can be an individual who may or may not be infected—and the sample could be any biological sample (e.g., blood, saliva, biopsy, plasma, serum, bronchoalveolar lavage, sputum, a fecal sample, cerebrospinal fluid, a fine needle aspirate, a swab sample (e.g., a buccal swab, a cervical swab, a nasal swab), interstitial fluid, synovial fluid, nasal discharge, tears, buffy coat, a mucous membrane sample, an epithelial cell sample (e.g., epithelial cell scraping), etc.) collected from the individual. In some embodiments, the sample is a cell-free liquid sample.

In some embodiments, the sample is a liquid sample that can comprise cells (urine, blood, serum, plasma, lymphatic fluid, cerebrospinal fluid, saliva, nasopharyngeal, oropharyngeal, nasopharyngeal/oropharyngeal, aspirate, and biopsy). Pathogens include viruses, fungi, helminths, protozoa, malarial parasites, Plasmodium parasites, Toxoplasma parasites, Schistosoma parasites, and the like. “Helminths” include roundworms, heartworms, and phytophagous nematodes (Nematoda), flukes (Tematoda), Acanthocephala, and tapeworms (Cestoda). Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, Plasmodium vivax, Trypanosoma cruzi and Toxoplasma gondii. Fungal pathogens include, but are not limited to: Cryptococcus neoformans, Histoplasma capsulatum, Coccidioides immitis, Blastomyces dermatitidis, Chlamydia trachomatis, and Candida albicans. Pathogenic viruses include RNA or DNA viruses, e.g., coronoavirus (e.g. SARS-CoV, SARS-CoV-2, MERS-CoV); immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogenic viruses can include DNA viruses such as: a papovavirus (e.g., human papillomavirus (HPV), polyomavirus); a hepadnavirus (e.g., Hepatitis B Virus (HBV)); a herpesvirus (e.g., herpes simplex virus (HSV), varicella zoster virus (VZV), epstein-barr virus (EBV), cytomegalovirus (CMV), herpes lymphotropic virus, Pityriasis Rosea, kaposi's sarcoma-associated herpesvirus); an adenovirus (e.g., atadenovirus, aviadenovirus, ichtadenovirus, mastadenovirus, siadenovirus); a poxvirus (e.g., smallpox, vaccinia virus, cowpox virus, monkeypox virus, orf virus, pseudocowpox, bovine papular stomatitis virus; tanapox virus, yaba monkey tumor virus; molluscum contagiosum virus (MCV)); a parvovirus (e.g., adeno-associated virus (AAV), Parvovirus B19, human bocavirus, bufavirus, human parv4 G1); Geminiviridae; Nanoviridae; Phycodnaviridae; and the like. Pathogens can include, e.g., DNAviruses [e.g.: a papovavirus (e.g., human papillomavirus (HPV), polyomavirus); a hepadnavirus (e.g., Hepatitis B Virus (HBV)); a herpesvirus (e.g., herpes simplex virus (HSV), varicella zoster virus (VZV), epstein-barr virus (EBV), cytomegalovirus (CMV), herpes lymphotropic virus, Pityriasis Rosea, kaposi's sarcoma-associated herpesvirus); an adenovirus (e.g., atadenovirus, aviadenovirus, ichtadenovirus, mastadenovirus, siadenovirus); a poxvirus (e.g., smallpox, vaccinia virus, cowpox virus, monkeypox virus, orf virus, pseudocowpox, bovine papular stomatitis virus; tanapox virus, yaba monkey tumor virus; molluscum contagiosum virus (MCV)); a parvovirus (e.g., adeno-associated virus (AAV), Parvovirus B19, human bocavirus, bufavirus, human parv4 G1); Geminiviridae; Nanoviridae; Phycodnaviridae; and the like], Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae.

c. Measuring a Detectable Signal

The detection method generally includes a step of measuring (e.g., measuring a detectable signal produced by the Type V or Type VI of the disclosure. A detectable signal can be any signal that is produced when ss oliogonucleotide is cleaved. The step of detection can involve a fluorescence-based detection. The readout of such detection methods can be any convenient readout. Examples of possible readouts include but are not limited to: a measured amount of detectable fluorescent signal; a visual analysis of bands on a gel (e.g., bands that represent cleaved product versus uncleaved substrate), a visual or sensor based detection of the presence or absence of a color (i.e., color detection method), the presence or absence of (or a particular amount of) a magnetic signal and the presence or absence of (or a particular amount of) an electrical signal.

The measuring can in some embodiments be quantitative, e.g., in the sense that the amount of signal detected can be used to determine the amount of target DNA or RNA present in the sample. The measuring can in some embodiments be qualitative, e.g., in the sense that the presence or absence of detectable signal can indicate the presence or absence of targeted DNA or RNA (e.g., virus, SNP, etc.). In some embodiments, a detectable signal will not be present (e.g., above a given threshold level) unless the targeted DNA or RNA(s) (e.g., virus, SNP, etc.) is present above a particular threshold concentration. In some embodiments, the threshold of detection can be titrated by modifying the amount of the Type V or Type VI endonuclease provided.

The compositions and methods of this disclosure can be used to detect any DNA or RNA target.

In some embodiments, the detection methods of the disclosure can be used to determine the amount of a target DNA or RNA in a sample (e.g., a sample comprising the target DNA or RNA and a plurality of non-target DNA or RNAs). Determining the amount of a target DNA or RNA in a sample can comprise comparing the amount of detectable signal generated from a test sample to the amount of detectable signal generated from a reference sample. Determining the amount of a target DNA or RNA in a sample can comprise: measuring the detectable signal to generate a test measurement; measuring a detectable signal produced by a reference sample to generate a reference measurement; and comparing the test measurement to the reference measurement to determine an amount of target DNA or RNA present in the sample.

In some embodiments, the detectable signal is detectable in less than 1, 2, 3, 4, 5, 10, 15, 20, 30, 60, 90, 120, 150, 180, 210, or 240 minutes.

In some embodiments, sensitivity of a subject composition and/or method (e.g., for detecting the presence of a target DNA or RNA, such as viral DNA or RNA or a SNP, in cellular genomic DNA or RNA) can be increased by coupling detection with nucleic acid amplification.

In some embodiments, the nucleic acids in a sample are amplified prior to contact with a Type V or Type VI; in particular embodiments, the Type V or Type VI remains in an inactive state until amplification has concluded. In some embodiments, the nucleic acids in a sample are amplified simultaneous with contact with Type V or Type VI. Amplification can be carried out using primers. As it relates to the overall processing time for the detection method, amplification can occur for 5 seconds or more, up to 240 minutes or more.

Various amplification methods and components will be known to one of ordinary skill in the art and any convenient method can be used.

Nucleic acid amplification can comprise polymerase chain reaction (PCR), reverse transcription PCR (RT-PCR), quantitative PCR (qPCR), reverse transcription qPCR (RT-qPCR), isothermal PCR, nested PCR, multiplex PCR, asymmetric PCR, touchdown PCR, random primer PCR, hemi-nested PCR, polymerase cycling assembly (PCA), colony PCR, ligase chain reaction (LCR), digital PCR, methylation specific-PCR (MSP), co-amplification at lower denaturation temperature-PCR (COLD-PCR), allele-specific PCR, intersequence-specific PCR (ISS-PCR), whole genome amplification (WGA), inverse PCR, and thermal asymmetric interlaced PCR (TAIL-PCR).

In some embodiments the amplification is isothermal amplification. Isothermal nucleic acid amplification methods can therefore be carried out inside or outside of a laboratory environment. Examples of isothermal amplification methods include but are not limited to: loop-mediated isothermal Amplification (LAMP), helicase-dependent Amplification (HDA), recombinase polymerase amplification (RPA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification (SPIA), signal mediated amplification of RNA technology (SMART), self-sustained sequence replication (3 SR), genome exponential amplification reaction (GEAR) and isothermal multiple displacement amplification (IMDA).

d. Detector Oligonucleotides

The novel Type V or Type VI endonucleases of the disclosure possess collateral cleavage (trans-cleavage) activity.

In some embodiments, a detection method includes contacting a sample with: i) a Type V or Type VI endonuclease of the disclosure; ii) a gRNA (or precursor gRNA array); and iii) a detector that does not hybridize with the guide sequence of the gRNA. For example, in some embodiments, a detection method includes contacting a sample with a labeled detector that includes a fluorescence-emitting dye pair; the Type V or Type VI endonuclease of the disclosure has the ability to cleave the labeled detector after it is activated (by gRNA hybridizing to a target DNA or RNA); and the detectable signal that is measured is produced by the fluorescence-emitting dye pair. For example, in some embodiments, a detection method includes contacting a sample with a labeled detector comprising a fluorescence resonance energy transfer (FRET) pair or a quencher/fluor pair, or both. In some embodiments, a detection method includes contacting a sample with a labeled detector comprising a FRET pair. In some embodiments, a detection method includes contacting a sample with a labeled detector comprising a fluor/quencher pair.

Fluorescence-emitting dye pairs comprise a FRET pair or a quencher/fluor pair. In both embodiments of a FRET pair and a quencher/fluor pair, the emission spectrum of one of the dyes overlaps a region of the absorption spectrum of the other dye in the pair. As used herein, the term “fluorescence-emitting dye pair” is a generic term used to encompass both a “fluorescence resonance energy transfer (FRET) pair” and a “quencher/fluor pair”. The term “fluorescence-emitting dye pair” is used interchangeably with the phrase “a FRET pair and/or a quencher/fluor pair.”

In some embodiments (e.g., when the detector includes a FRET pair) the labeled detector produces an amount of detectable signal prior to being cleaved, and the amount of detectable signal that is measured is reduced when the labeled detector is cleaved. In some embodiments, the labeled detector produces a first detectable signal prior to being cleaved (e.g., from a FRET pair) and a second detectable signal when the labeled detector is cleaved (e.g., from a quencher/fluor pair). As such, in some embodiments, the labeled detector comprises a FRET pair and a quencher/fluor pair.

In some embodiments, the labeled detector comprises a FRET pair.

FRET donor and acceptor moieties (FRET pairs) will be known to one of ordinary skill in the art and any convenient FRET pair (e.g., any convenient donor and acceptor moiety pair) can be used. Examples of suitable FRET pairs include but are not limited to those presented in Table 11. FRET pairs provided in U.S. Pat. No. 10,253,365 are incorporate by reference herein in their entirety. In some embodiments, the FRET pair is 5′ 6-FAM and 3IABkFQ (Iowa Black (Registered)-FQ).

TABLE 11
Examples of FRET pairs (donor and and acceptor pairs)

| Donor | Acceptor |
|---|---|
| Tryptophan | Dansyl |
| IAEDANS (1) | DDPM (2) |
| BFP | DsRFP |
| Dansyl | Fluorescein |
| | isothiocyanate (FITC) |
| Dansyl | Octadecylrhodamine |
| Cyan fluorescent | Green fluorescent protein |
| protein (CFP) | (GFP) |
| CF (3) | Texas Red |
| Fluorescein | Tetramethylrhodamine |
| Cy3 | Cy5 |
| GFP | Yellow fluorescent |
| | protein (YFP) |
| BODIPY FL (4) | BODIPY FL (4) |
| Rhodamine 110 | Cy3 |
| Rhodamine 6G | Malachite Green |
| FITC | Eosin Thiosemicarbazide |
| B-Phycoerythrin | Cy5 |
| Cy5 | Cy5.5 |

(1) 5-(2-iodoacetylaminoethyl)aminonaphthalene-1-sulfonic
acid
(2) N-(4-dimethylamino-3,5-dinitrophenyl)maleimide
(3) carboxyfluorescein succinimidyl ester
(4) 4,4-difluoro-4-bora-3a,4a-diaza-s-indacene

In some embodiments, a detectable signal is produced when the labeled detector is cleaved (e.g., in some embodiments, the labeled detector comprises a quencher/fluor pair).

Any fluorescent label can be utilized. Examples of fluorescent labels include, but are not limited to: an Alexa Fluor® dye, an ATTO dye (e.g., ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATTO 740), a DyLight dye, a cyanine dye (e.g., Cy2, Cy3, Cy3.5, Cy3b, Cy5, Cy5.5, Cy7, Cy7.5), a FluoProbes dye, a Sulfo Cy dye, a Seta dye, an IRIS Dye, a SeTau dye, an SRfluor dye, a Square dye, fluorescein isothiocyanate (FITC), fluorescein amidite (FAM), tetramethylrhodamine (TRITC), Texas Red, Oregon Green, Pacific Blue, Pacific Green, Pacific Orange, quantum dots, and a tethered fluorescent protein.

Examples of quencher moieties include, but are not limited to: a dark quencher, a Black Hole Quencher® (BHQ®) (e.g., BHQ-0, BHQ-1, BHQ-2, BHQ-3), a Qxl quencher, an ATTO quencher (e.g., ATTO 540Q, ATTO 580Q, and ATTO 612Q), dimethylaminoazobenzenesulfonic acid (Dabsyl), Iowa Black RQ, Iowa Black FQ, IRDye QC-1, a QSY dye (e.g., QSY 7, QSY 9, QSY 21), AbsoluteQuencher, Eclipse, and metal clusters such as gold nanoparticles, and the like.

In some embodiments, a quencher moiety is selected from: a dark quencher, a Black Hole Quencher® (BHQ®) (e.g., BHQ-0, BHQ-1, BHQ-2, BHQ-3), a Qxl quencher, an ATTO quencher (e.g., ATTO 540Q, ATTO 580Q, and ATTO 612Q), dimethylaminoazobenzenesulfonic acid (Dabsyl), Iowa Black RQ, Iowa Black FQ, IRDye QC-1, a QSY dye (e.g., QSY 7, QSY 9, QSY 21), AbsoluteQuencher, Eclipse, and a metal cluster.

In some embodiments, cleavage of a labeled detector can be detected by measuring a colorimetric read-out. For example, the liberation of a fluorophore (e.g., liberation from a FRET pair, liberation from a quencher/fluor pair, and the like) can result in a wavelength shift (and thus color shift) of a detectable signal. Thus, in some embodiments, cleavage of a subject labeled detector can be detected by a color-shift. Such a shift can be expressed as a loss of an amount of signal of one color (wavelength), a gain in the amount of another color, a change in the ration of one color to another, and the like.

As provided herein, a labeled detector can be a nucleic acid mimetic. Polynucleotide mimics include PNAs, LNAs, CeNAs, and morpholino nucleic acids.

A labeled detector can also include one or more substituted sugar moieties.

A labeled detector may also include modified nucleotides.

e. Positive Controls

The detection methods provided herein can also include a positive control target DNA or RNA. In some embodiments, the methods include using a positive control gRNA that comprises a nucleotide sequence that hybridizes to a control target DNA or RNA. In some embodiments, the positive control target DNA or RNA is provided in various amounts. In some embodiments, the positive control target DNA or RNA is provided in various known concentrations, along with control non-target DNA or RNAs.

f. gRNA Arrays

In some embodiments, the method comprises contacting the sample with a precursor gRNA array, wherein the novel Type V or Type VI endonuclease of the disclosure cleaves the precursor gRNA array to produce said gRNA.

In some embodiments a such a gRNA array includes 2 or more gRNAs (e.g., 3 or more, 4 or more, 5 or more, 6 or more, or 7 or more, gRNAs). The gRNAs of a given array can target (i.e., can include guide sequences that hybridize to) different target sites of the same target DNA or RNA (e.g., which can increase sensitivity of detection) and/or can target different target DNA or RNAs (e.g., single nucleotide polymorphisms (SNPs), different strains of a particular virus, etc.), and such could be used for example to detect multiple strains of a virus. In some embodiments, each gRNA of a precursor gRNA array has a different guide sequence.

In some embodiments, the precursor gRNA array comprises two or more gRNAs that target different target sites within the same target DNA or RNA. For example, such a scenario can in some embodiments increase sensitivity of detection by activating Type II, Type V or Type VI endonuclease of the disclosure when either one hybridizes to the target DNA or RNA. As such, in some embodiments as subject composition (e.g., kit) or method includes two or more gRNAs (in the context of a precursor gRNA array, or not in the context of a precursor gRNA array, e.g., the gRNAs can be mature gRNAs).

In some embodiments, the precursor gRNA array comprises two or more gRNAs that target different target DNA or RNAs. For example, such a scenario can result in a positive signal when any one of a family of potential target DNA or RNAs is present. Such an array could be used for targeting a family of transcripts, e.g., based on variation such as single nucleotide polymorphisms (SNPs) (e.g., for diagnostic purposes). Such could also be useful for detecting whether any one of a number of different strains of virus is present. Such could also be useful for detecting whether any one of a number of different species, strains, isolates, or variants of a bacterium or virus is present As such, in some embodiments as subject composition (e.g., kit) or method includes two or more gRNAs (in the context of a precursor gRNA array, or not in the context of a precursor gRNA array, e.g., the gRNAs can be mature gRNAs).

### VI. Compositions of Matter

Provided herein are compositions and pharmaceutical compositions comprising the Type II, Type V, or Type VI endonucleases and/or the Type II, Type V, or Type VI gRNAs of the disclosure, which can optionally include a pharmaceutically acceptable carrier and/or a protein stabilizing buffer, and/or a nucleic acid stabilizing buffer. In some embodiments, the Type II, Type V, or Type VI endonucleases and/or the Type II, Type V, or Type VI gRNAs are provided in a lyophilized form.

Provided herein are compositions comprising gRNAs and/or gRNA arrays of the disclosure (compatible for use with Type II, Type V, or Type VI endonucleases of the disclosure), and optionally a protein stabilizing buffer.

Provided herein are proteins comprising an amino acid sequence with 30%-99.5% homology to any one of SEQ ID NOs: 1-20. Provided herein are compositions comprising these proteins, and optionally a pharmaceutically acceptable carrier. Provided herein are these proteins and optionally a protein stabilizing buffer.

Provided herein are DNA polynucleotides encoding a sequence that encodes any of the Type II, Type V, or Type VI endonucleases of the disclosure. Also provided are recombinant expression vectors comprising such DNA polynucleotides. In some embodiments, a nucleotide sequence encoding a Type II, Type V, or Type VI endonuclease of the disclosure is operably linked to a promoter. In some embodiments, the nucleic acid encoding the Type II, Type V, or Type VI endonuclease further comprises a nuclear localization signal (NLS), useful for expression in eukaryotic systems.

Provided herein are DNA polynucleotides or RNAs comprising a sequence that encodes any of the gRNAs of the disclosure. Also provided are recombinant expression vectors comprising such DNA polynucleotides. In some embodiments, a nucleotide sequence encoding a gRNA of the disclosure is operably linked to a promoter.

Also provided herein are host cells comprising any of the recombinant vectors provided herein.

### VII. Kits

Provided herein are kits comprising one or more components of the Type II, Type V, and Type VI engineered systems described herein, useful for a variety of applications including, but not limited to, therapeutic and diagnostic applications.

In some embodiments provided herein is a kit comprising: (a) Type II endonuclease of the disclosure, or a nucleic acid encoding the Type II endonuclease; and (b) Type II gRNA, wherein the gRNA and the Type II endonuclease do not naturally occur together, wherein the gRNA is capable of hybridizing to a target sequence in a target DNA, and the gRNA is capable of forming a complex with the Type II endonuclease.

In some embodiments provided herein is a kit comprising: (a) Type V endonuclease, or a nucleic acid encoding the Type V endonuclease; and (b) Type V gRNA, wherein the gRNA and the Type V endonuclease do not naturally occur together, wherein the gRNA is capable of hybridizing to a target sequence in a single stranded or double stranded target DNA, and the gRNA is capable of forming a complex with the Type II endonuclease.

In exemplary embodiments, provided herein are diagnostic kits. In exemplary embodiments, the reagent components are provided in lyophilized form. In some embodiments, the reagent components are provided individually (either lyophilized or not lyophilized), in other embodiments, the reagent components are provided in a pre-mixed format (either lyophilized or not lyophilized).

By way of example only, the following are exemplary kit reagent components useful for the detection of SARS-CoV-2, a RNA virus, using one of the novel Type V or Type VI endonucleases of the disclosure.

(1) Lyophilized reaction mix containing reagents, SARS-CoV-2 primer sets and enzymes for reverse transcription and loop-mediated isothermal amplification (RT-LAMP) of a gene of diseasSARS-CoV-2 genome.

(2) Lyophilized reaction mix containing reagents, control RNAse P primer sets and enzymes for reverse transcription and RT-LAMP amplification of human housekeeping gene RNAse P.

(3) Lyophilized reaction mix containing reagents and CRISPR-Cas enzyme gRNA-RNP complexes for detection of a SARS-CoV-2 amplification product. Such mix may also include a labeled reporter, e.g. a 5′FAM-3′Quencher ssRNA or ssDNA-based oligonucleotide reporter, or a 5′FAM-3′Quencher single stranded DNA/RNA chimera-based oligonucleotide reporter.

(4) Lyophilized reaction mix containing reagents and Cas enzyme gRNA-RNP complexes for detection of RNAse P amplification product. Such mix may also include a labeled reporter, e.g. a 5′FAM-3′Quencher RNA-based oligonucleotide reporter.

### EXAMPLES

The following examples are included for illustrative purposes and are not intend to limit the scope of the invention.

### Example 1: Identification and Expression of Novel Endonucleases

Metagenome sequences were obtained from environmental samples, and compiled to construct a database of putative CRISPR-Cas loci. CRISPR arrays were identified using CrisprCasFinder software. The criteria of filtering were putative Class II Type II, V, and VI effectors >400 aa, which were adjacent to cas genes and CRISPR arrays. Sequences were aligned with Clustal Omega using HMM profiles. Genes were identified from metagenomic samples. Scripts were run on the sequences, designed to find CRISPR sequences and accompanying genes encoding proteins showing homology with reported Cas enzymes. Comparative BlastP analyses were performed against sequences deposited in databases (NCBI, LENS), discarding those candidates showing Id % >50 with deposited proteins. Presence of specific domains (e.g. RuvC, HEPN) and catalytic motifs were determined (CD-search, phmmer, UNIPROT). The novel endonucleases described herein were identified.

Expression vectors were artificially synthesized. The effector plasmid codon optimization, synthesis, and cloning were generated. Expression plasmids were transformed into E. coli.

### Example 2: Characterization of Type V Cas_1

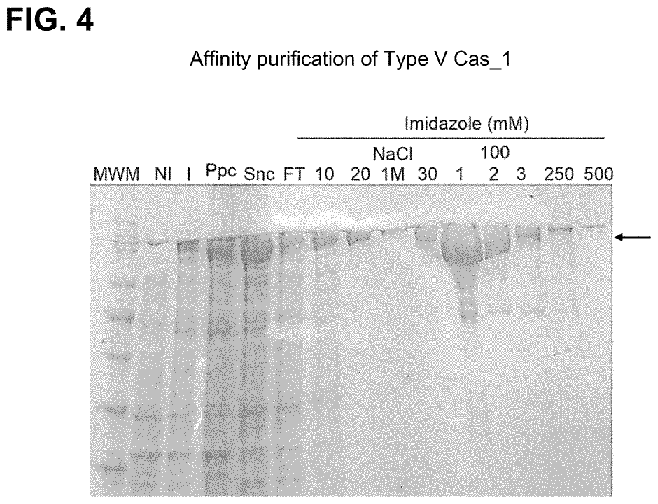

SEQ ID NO: 1 represents a novel Type V variant of the disclosure, Type V Cas_1, (1283 amino acids in length). FIG. 4 shows the molecular weight and purity using SDS-PAGE after protein purification.

The Type V Cas_1 protein was purified via the following scheme. Recombinant protein was expressed in E. coli NiCo21 (DE3) cells (NEB #C2529H) harboring the pET28a/Type V Cas_1-H6X expression plasmid by growing in LB broth culture medium at 37° C. followed by induction of expression at 28° C. for 3 hr in presence of 0.25 mM IPTG. Cells were disrupted by sonication prior to chromatographic purification. Recombinant protein was purified using a HisTrapHP (Ni-NTA) (GE Healthcare) followed by a HiPrep™ 26/10 desalting column (GE Healthcare) where the protein was desalted into storage buffer containing Tris-HCl 50 mM (pH 8), NaCl 200 mM, MgCl2 20 mM, DTT 1 mM. Protein purity was controlled by Coomassie blue staining after SDS—PAGE on a 10% polyacrylamide gel. Protein concentrations were determined by UV spectroscopy and Qubit protein assay (Invitrogen). Purified proteins were stored at −80° C.

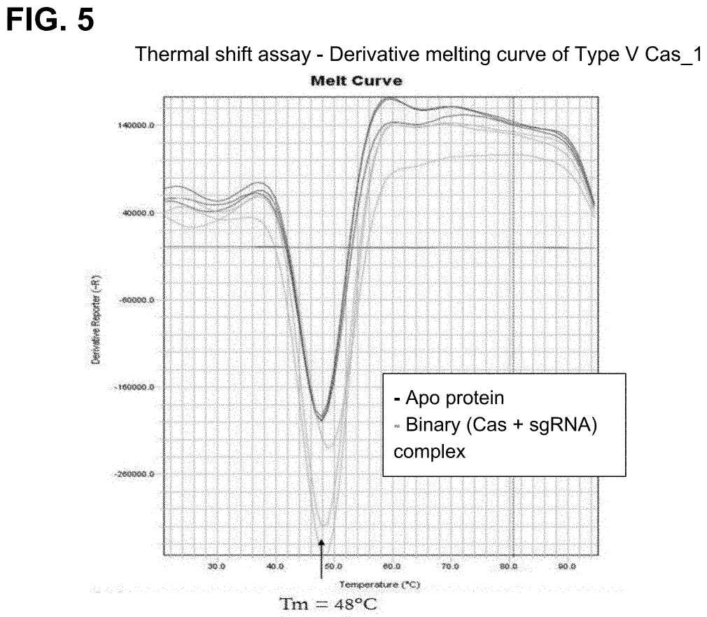

FIG. 5 shows the results of a temperature-based assay to assess the stability of the Type V Cas_1 protein. The first derivative plots of the melting curve display the thermostability of apo protein form and its binary complex (Type V Cas_1+sgRNA). The melting curve was obtained using Sypro Orange thermal shift (Invitrogen). The first derivative plots of Type V Cas_1 and its binary complex are nearly overlapping [melting temperature (Tm)=48-49° C.]. All reactions were performed (apo protein or complex) to a final concentration of 1.8 μM in buffer (50 mM Tris-HCl pH 8, 200 mM NaCl, 20 mM MgCl2, 1 mM DTT) with the addition of 10× SYPRO Orange (Invitrogen). Binary complex (protein+sgRNA) was formed at a 1:1 ratio. Apo and complexes were incubated at room temperature for 10 minutes prior to melting to assure complex formation. The reactions were then split into three 20 uL technical replicates. Protein melting assay was performed in a StepOne™ Real-Time PCR System (Thermo Fisher) over a temperature range from 20° C. to 95° C., at a rate of 1° C./minute, with 1 acquisitions/minute. The first derivative of the raw fluorescence data was taken in order to determine the Tm of the protein.

FIG. 6 shows the Type V Cas_1 trans-cleavage activity on single-stranded DNA reporter. The specificity of trans-cleavage activity was tested using customized ssDNA 5′6-FAM TTATTATT-3IABkFQ3′ from IDT (Integrated DNA Technologies, Inc) as reporter. The results show that Type V Cas_1 is able to cleave the ssDNA reporter used. The detection assay was performed at 37° C. using Type V Cas_1 complexes to a final concentration of 75 nM Cas:75 nM sgRNA:10 nM activator in a solution containing 1× Binding Buffer (50 mM NaCl, 10 mM Tris-HCl, 10 mM MgCl2, 1 mM DTT, 100 g/ml BSA, pH 7.9) and 600 nM of ssDNA FAMQ reporter substrate in a 40 μl reaction. Reactions (40 μl, 384-well microplate format) were incubated in a fluorescence plate reader (SpectraMax® M2) for 40 minutes at 37° C. with fluorescence measurements taken every 1 minute (ssDNA FQ substrates=λex: 485 nm; λem: 538 nm). Non-template negative control (NTC) fluorescence values were calculated from reactions carried out in the absence of Hanta target. Error bars represent the mean±s.d., where n=3 replicates. A) time.

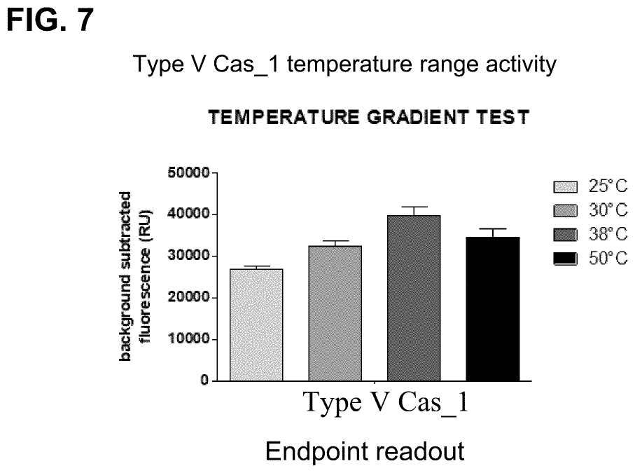

FIG. 7 shows the activity of Type V Cas_1 protein at different temperatures (25° C.-50° C.). The efficiency of trans-cleavage activity at different temperatures was tested using customized ssDNA 5′6-FAM TTATTATT-3IABkFQ3′ from IDT (Integrated DNA Technologies, Inc) as a reporter. The results showed that Type V Cas_1 is able to cleave with similar efficiency the ssDNA reporter in a wide range from room temperature even as high as 50° C. The detection assay was performed at 25° C., 30° C., 38° C. and 50° C. using Type V Cas_1 complexes to a final concentration of 75 nM Cas:75 nM sgRNA:10 nM activator in a solution containing 1× Binding Buffer (50 mM NaCl, 10 mM Tris-HCl, 10 mM MgCl2, 1 mM DTT, 100 g/ml BSA, pH 7.9) and 600 nM of ssDNA FAMQ reporter substrate in a 40₁1.1 reaction. Reactions (40 μl, 384-well microplate format) were incubated in a thermocycler for 20 minutes at 25, 30, 38 or 50° C. and then endpoint measures were taken in a fluorescence plate reader SpectraMax® M2 (ssDNA FQ substrates=λex: 485 nm; λem: 538 nm). Background-corrected fluorescence values were calculated by subtracting fluorescence values obtained from reactions carried out in the absence of target plasmid. Error bars represent the mean±s.d., where n=3 replicates.

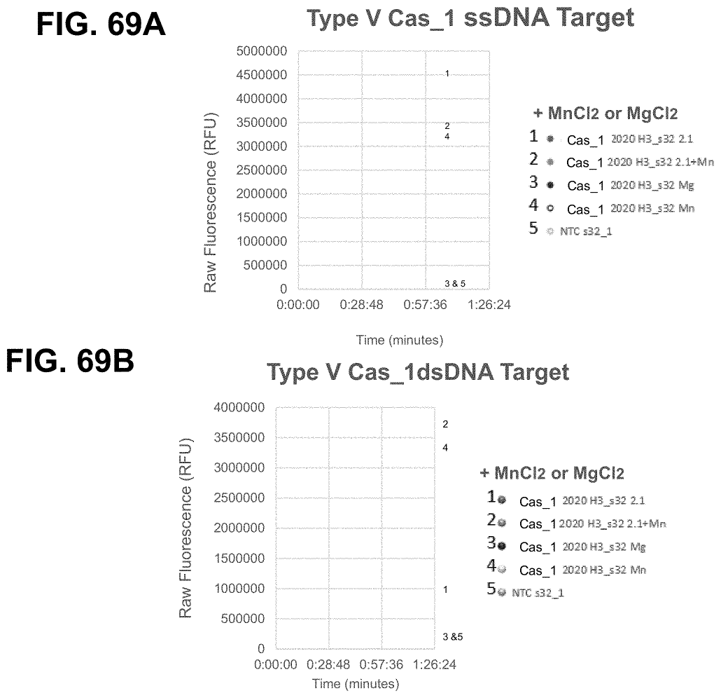

FIGS. 69A-69B show collateral activity for Type V Cas_1 protein complex using as substrate a single-stranded DNA (IDT primer) (FIG. 69A) and (B) (FIG. 69B) double-stranded DNA (customized plasmid containing Hanta sequence). The activity was measured at 37° C. for 1 h in presence of MnCl₂ and/or MgCl₂. The addition of manganese increase the speed of the reaction and is essential when using dsDNA as target. The reaction was initiated by preparing complexes to a final concentration of 150 nM Type V Cas_1: 150 nM sgRNA: 10 nM activator or 10 nM of double-stranded DNA in a solution containing 1× Binding Buffer (50 mM NaCl, 10 mM Tris-HCl, 1 mM DTT, 100 g/ml BSA, 10 mM of MgCl₂ and/or 10 nM MnCl₂, pH 7.9). The specificity of trans-cleavage activity was tested using customized ssDNA/56-FAM/TTATTATT/3IABkFQ/from IDT (Integrated DNA Technologies, Inc.) as reporter. Control groups without Cas enzyme, guide or target were included and non-collateral cleavage was observed.

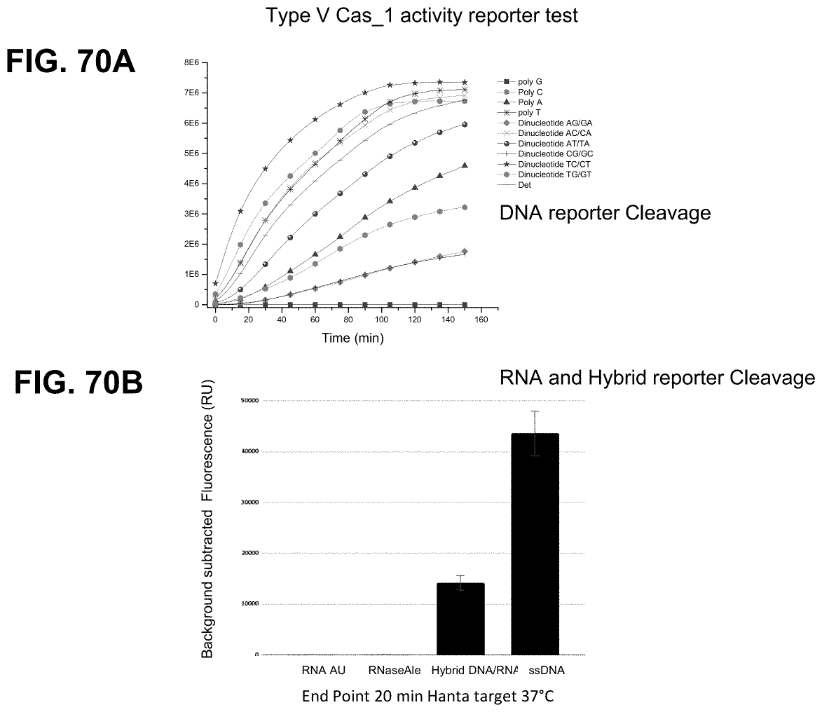

FIGS. 70A and 70B show trans-cleavage activities on single-stranded reporters. We tested the specificity of trans-cleavage activity using customized ssDNA, Hybrid DNA/RNA, ssRNA AU and RNaseAlert™ from IDT (Integrated DNA Technologies, Inc.) as reporters. The results showed that Type V Cas_1 protein is able to cleave DNA or Hybrid reporters used but not the RNA reporters tested. Detection assays were performed at 37° C. using Type V Cas_1 complex to a final concentration of 150 nM Type V Cas_1: 150 nM sgRNA: 10 nM activator in a solution containing 1× Binding Buffer (50 mM NaCl, 10 mM Tris-HCl, 10 mM MgCl2, 1 mM DTT, 100 g/ml BSA, pH 7.9) and 600 nM of FAMQ reporter substrates (ssRNA 5′6-FAM rArUrArUrArUrA-3IABkFQ3, RNaseAlert (Cat N 11-04-03-03-IDT, ssDNA (/56-FAM/TTATTATT/3IABkFQ/) and Hybrid DNA/RNA (/56-FAM/TTATrUrArUrU/3IABkFQ/) in a 40 μl reaction. Reactions were incubated in a fluorescence plate reader (SpectraMax®M2) and background-corrected fluorescence values were calculated by subtracting fluorescence values obtained from reactions carried out in the absence of target plasmid. Error bars represent the mean±s.d., where n=3 replicates. Results are shown in FIGS. 70A and 70B.

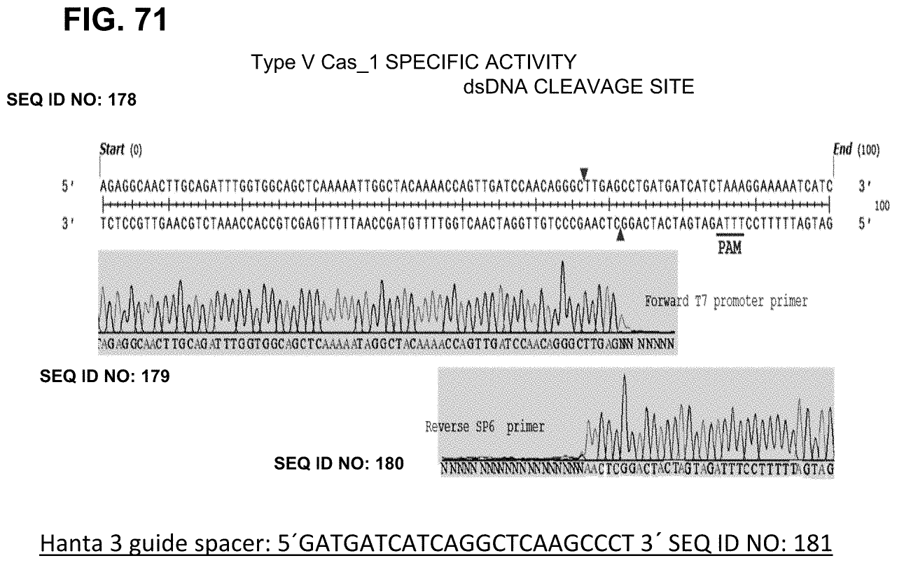

FIG. 71 shows the specific activity for dsDNA cleavage site determination. The results showed that Type V Cas_1 protein cuts at the 13th base site of the non-complementary strand and the 18th base site of the complementary strand downstream of the PAM sequence, generating a 5-nt overhang when the spacer length is 23 nt. Experiments were performed at 37° C. using Type V Cas_1 complex to a final concentration of 500 nM Type V Cas_1: 500 nM sgRNA: pGEM-T easy/Hanta dsDNA, 3 μg in a solution containing 1× Binding Buffer (50 mM NaCl, 10 mM Tris-HCl, 10 mM MgCl2, 1 mM DTT, 100 g/ml BSA, pH 7.9). Reactions were incubated 4 hours and the product was sent to a sequencing service. Detection assays were performed at 37° C. using Type V Cas_1 complex to a final concentration of 150 nM Type V Cas_1: 150 nM sgRNA: 10 nM activator in a solution containing 1× Binding Buffer (50 mM NaCl, 10 mM Tris-HCl, 10 mM MgCl2, 1 mM DTT, 100 g/ml BSA, pH 7.9) and 600 nM of FAMQ reporter substrates (ssRNA 5′6-FAM rArUrArUrArUrA-3IABkFQ3, RNaseAlert (Cat N 11-04-03-03-IDT, ssDNA (/56-FAM/TTATTATT/3IABkFQ/)) and Hybrid DNA/RNA (/56-FAM/TTATrUrArUrU/3IABkFQ/) in a 40 μl reaction. Reactions were incubated in a fluorescence plate reader (SpectraMax®M2) and background-corrected fluorescence values were calculated by subtracting fluorescence values obtained from reactions carried out in the absence of target plasmid. Error bars represent the mean±s.d., where n=3 replicates. Results are shown in FIG. 71.

### Example 2: Characterization of Type V Cas_2

SEQ ID NO: 2 represents a novel Type V variant of the disclosure, Type V Cas_2, (1235 amino acids in length). FIG. 8 is a schematic representation of the organization of the CRISPR Cas cluster loci around the novel Type V Cas_2 gene of the disclosure. FIG. 10 shows the amino acid sequence of Type V Cas_2 with the RuvC motifs underlined/highlighted (SEQ ID NO: 2). The FnType V sequence referenced in Shmakov et al., 2015 was used as a reference for identification of the Ruv motifs.

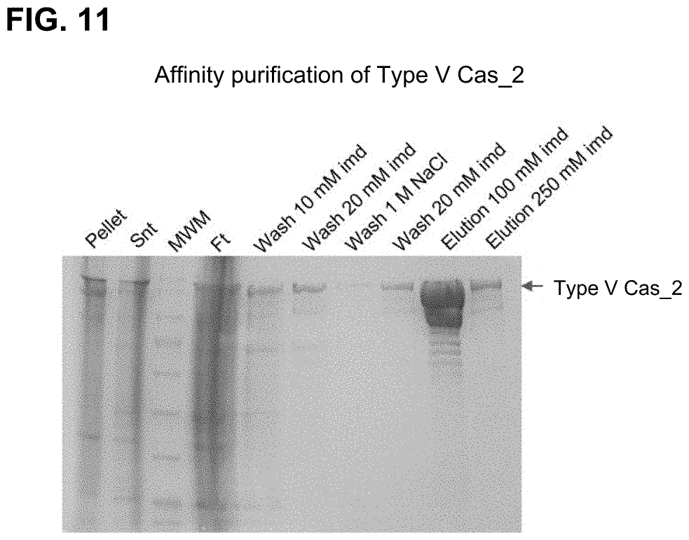

FIG. 11 shows Type V Cas_2 molecular weight and purity using SDS-PAGE after protein purification. Recombinant protein was expressed in E. coli Rosetta (DE3) cells (Novagen #70954) harboring the pET28a(+)-TEV/Cas expression plasmid by growing in LB broth culture medium at 37° C. followed by induction of expression at 28° C. for 6 hr in presence of 0.25 mM IPTG. Cells were disrupted by sonication prior to chromatographic purification. Recombinant protein was purified using a HisTrapHP (Ni-NTA) (GE Healthcare) followed by a HiPrep™ 26/10 desalting column (GE Healthcare) where the protein was desalted into storage buffer containing Tris-HCl 50 mM (pH 8), NaCl 200 mM, MgCl2 20 mM, DTT 1 mM. Protein purity was controlled by Coomassie blue staining after SDS-PAGE on a 10% polyacrylamide gel. Protein concentrations were determined by UV spectroscopy and Qubit protein assay (Invitrogen). Purified proteins were stored at −80° C.

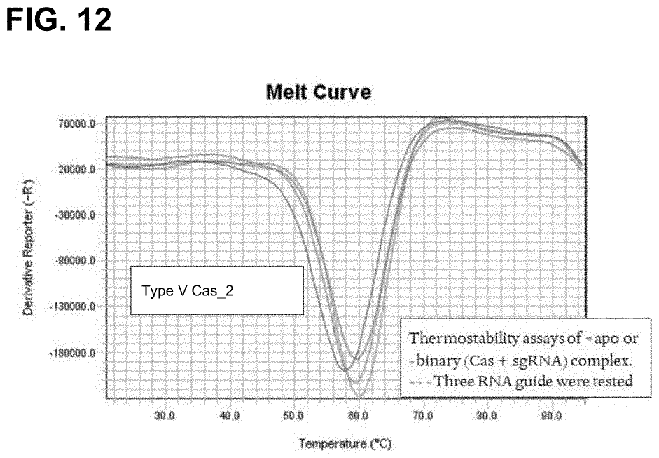

FIG. 12 shows that the protein Type V Cas_2 and its binary complex (Type V Cas_2+sgRNA) are thermostable. The first derivative plots of melting curve displaying the thermostability of apo protein form and binary complex. The melting curve was obtained by a thermal shift assay using Sypro Orange (Invitrogen). The first derivative plots of Type V Cas_2 and binary complex are nearly overlapping [melting temperature (Tm)=59-60° C.]. All reactions were performed (apo protein or complex) to a final concentration of 1.8 μM in buffer (50 mM Tris-HCl pH 8, 200 mM NaCl, 20 mM MgCl2, 1 mM DTT) with the addition of 10× SYPRO Orange (Invitrogen). Binary complex (protein+sgRNA) was formed at a 1:1 ratio. Apo and complexes were incubated at room temperature for 10 minutes prior to melting to assure complex formation. The reactions were then split into three 20 uL technical replicates. Protein melting assay was performed in a StepOne™ Real-Time PCR System (Thermo Fisher) over a temperature range from 20° C. to 95° C., at a rate of 1° C./minute, with 1 acquisitions/minute. The first derivative of the raw fluorescence data was taken in order to determine the Tm of the protein.

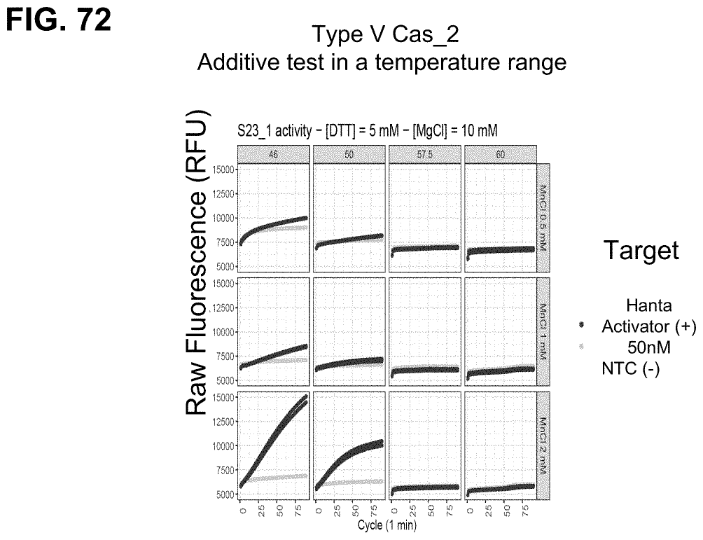

FIG. 72 shows trans-cleavage activity testing DTT and MnCl₂ as additives in a temperature range (46° C.-60° C.). The efficiency of trans-cleavage activity at different temperatures was tested using customized ssDNA 5′6-FAM TTATTATT-3IABkFQ3′ from IDT (Integrated DNA Technologies, Inc.) as a reporter. High MnCl₂ concentrations are detrimental for activity, lower concentrations were tested in a wider range of temperatures. DTT was increased at 5 mM to prevent manganese oxidation. At lower temperature 2 mM of MnCl₂ presented the higher activities.

Detection assay was performed at 46° C., 50° C., 52.5° C. and 60° C. using Type V Cas_2 complexes to a final concentration of 150 nM Type V Cas_2: 150 nM sgRNA: 50 nM activator in a solution containing 1× Binding Buffer (25 mM NaCl, 10 mM Tris-HCl, 10 mM MgCl2, 5 mM DTT, 100 g/ml BSA, pH 8.8, MnCl₂ 0.5, 1, 2 mM) and 600 nM of ssDNA FAMQ reporter substrate in a 40 μl reaction. Reactions were incubated in a qPCR (Bio-Rad) for 100 minutes with fluorescence measurements taken every 1 minute (ssDNA FQ substrates=λex: 485 nm; λem: 538 nm). Non-template negative control (NTC) fluorescence values were calculated from reactions carried out in the absence of ssDNA Hanta target. Results are shown in FIG. 72.

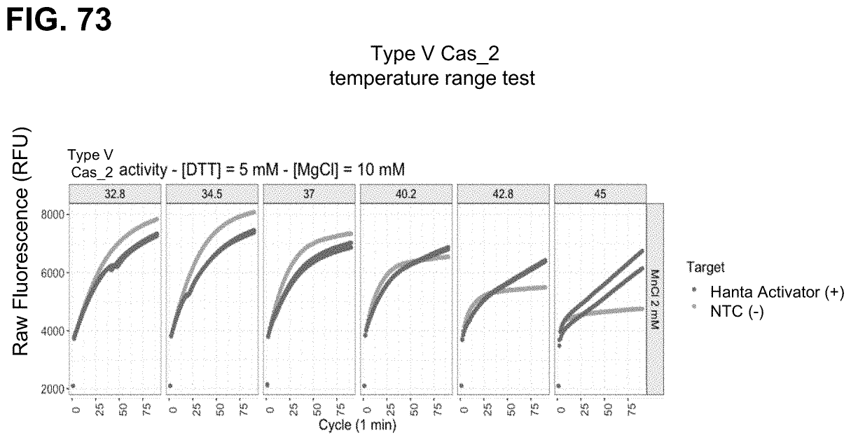

FIG. 73 shows the activity of Type V Cas_2 protein in a temperature curve (32.8° C.-45° C.). The efficiency of trans-cleavage activity at different temperatures was tested using customized ssDNA/56-FAM/TTATTATT/3IABkFQ/from IDT (Integrated DNA Technologies, Inc.) as a reporter. The results showed that Type V Cas_2 is able to cleave with low efficiency the ssDNA reporter only between 42.8° C. and 45° C. Detection assay was performed at 32.8° C., 34.5° C., 37° C., 40.2° C., 42.8° C. and 45° C. using Type V Cas_2 complexes to a final concentration of 150 nM Type V Cas_2: 150 nM sgRNA: 50 nM activator in a solution containing 1× Binding Buffer (25 mM NaCl, 10 mM Tris-HCl, 10 mM MgCl2, 5 mM DTT, 100 g/ml BSA, pH 8.8, 2 mM MnCl₂) and 600 nM of ssDNA FAMQ reporter substrate in a 40 μl reaction. Reactions were incubated in a qPCR (Bio-Rad) for 100 minutes with fluorescence measurements taken every 1 minute (ssDNA FQ substrates=λex: 485 nm; λem: 538 nm). Non-template negative control (NTC) fluorescence values were calculated from reactions carried out in the absence of ssDNA Hanta target. Results are shown in FIG. 73.

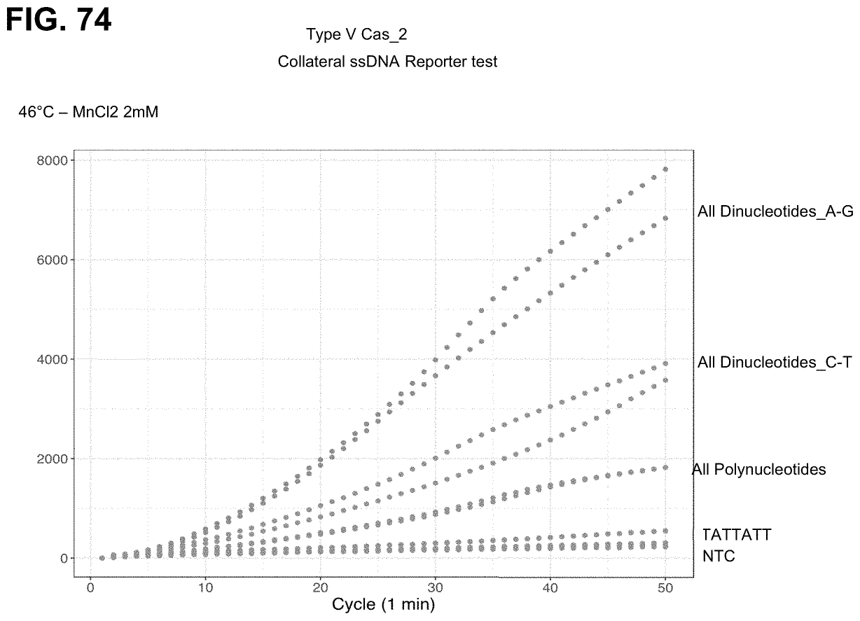

FIG. 74 shows differential efficiency in dinucleotide reporter cleavage. Different reporter sequences were tested showing a significant increase in Type V Cas_2 activity. This enzyme has demonstrated a highly efficiency in All Dinucleotide A-G cleavage, evidenced by increased fluorescence in compare with ssDNA determined FAMQ TTATTATT reporter sequence. Experiments were performed at 46° C. using Type V Cas_2 complex to a final concentration of 150 nM Type V Cas_2: 150 nM sgRNA: 10 nM ssDNA Hanta target, in a solution containing 1× Binding Buffer (25 mM NaCl, 10 mM Tris-HCl, 10 mM MgCl2, 5 mM DTT, 100 g/ml BSA, 2 mM MnCl₂, pH 8.8) and 1.25 μM of customized FAMQ reporter substrates (/56-FAM/TTATTATT/3IABkFQ/, All Dinucleotide_A-G/56 FAM/ATACAGAGTGCG/3IABkFQ/(SEQ ID NO: 143), All Dinucleotide_CT/56-FAM/TATGTCTCACGC/3IABkFQ/(SEQ ID NO: 144) and Poly Nucleotide All Polynucleotides/56-FAM/AAATTTCCCGGG/3IABkFQ/(SEQ ID NO: 145) (12 nt) from IDT (Integrated DNA Technologies, Inc.)) in a 40 μl reaction. Reactions were incubated in a qPCR (Bio-Rad) for 100 minutes with fluorescence measurements taken every 1 minute (ssDNA FQ substrates=λex: 485 nm; λem: 538 nm). Non-template negative control (NTC) fluorescence values were calculated from reactions carried out in the absence of ssDNA Hanta target. Results are shown in FIG. 74.

### Example 3: Characterization of Type V Cas_7

SEQ ID NO: 7 represents a novel Type V variant of the disclosure, Type V Cas_7, (1245 amino acids in length). FIG. 25 is a schematic representation of the organization of the CRISPR Cas cluster loci around the novel Type V Cas_7 gene of the disclosure. FIG. 27 shows the amino acid sequence of Type V Cas_7 with the RuvC motifs underlined/highlighted (SEQ ID NO: 7). The FnType V sequence referenced in Shmakov et al., 2015 was used as a reference for identification of the Ruv motifs.

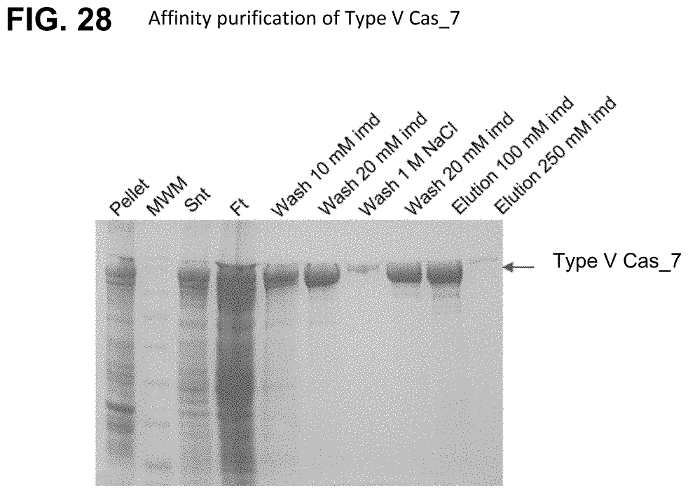

FIG. 28 shoes Type V Cas_7's molecular weight and purity through SDS-PAGE. The protein was purified via the following scheme. Recombinant protein was expressed in E. coli NiCo21 (DE3) cells (NEB #C2529H) harboring the pET28a/Type V Cas_7-H6X expression plasmid by growing in LB broth culture medium at 37° C. followed by induction of expression at 28° C. for 6 hr in presence of 0.25 mM IPTG. Cells were disrupted by sonication prior to chromatographic purification. Recombinant protein was purified using a HisTrapHP (Ni-NTA) (GE Healthcare) followed by a HiPrep™ 26/10 desalting column (GE Healthcare) where the protein was desalted into storage buffer containing Tris-HCl 50 mM (pH 8), NaCl 200 mM, MgCl2 20 mM, DTT 1 mM. Protein purity was controlled by Coomassie blue staining after SDS-PAGE on a 10% polyacrylamide gel. Protein concentrations were determined by UV spectroscopy and Qubit protein assay (Invitrogen). Purified proteins were stored at −80° C.

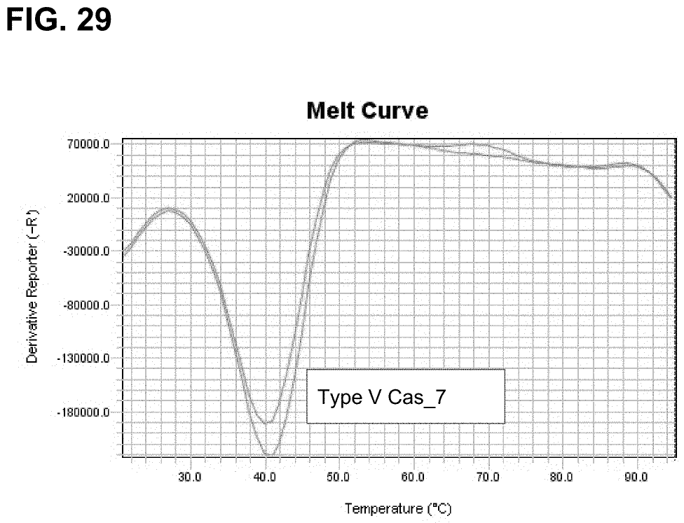

FIG. 29 shows the results of a temperature-based assay to assess the stability of Type V Cas_7 protein. The first derivative plots of the melting curve displaying the thermostability of apo protein form and its binary complex (Type V Cas_7+sgRNA). Melting curve was obtained by a thermal shift assay using Sypro Orange (Invitrogen). The first derivative plots of Type V Cas_7 and its binary complex are nearly overlapping [melting temperature (Tm)=40-41° C.]. All reactions were performed (apo protein or complex) to a final concentration of 1.8 μM in buffer (50 mM Tris-HCl pH 8, 200 mM NaCl, 20 mM MgCl2, 1 mM DTT) with the addition of 10× SYPRO Orange (Invitrogen). Binary complex (protein+sgRNA) was formed at a 1:1 ratio. Apo and complexes were incubated at room temperature for 10 minutes prior to melting to assure complex formation. The reactions were then split into three 20 uL technical replicates. Protein melting assay was performed in a StepOne™ Real-Time PCR System (Thermo Fisher) over a temperature range from 20° C. to 95° C., at a rate of 1° C./minute, with 1 acquisitions/minute. The first derivative of the raw fluorescence data was taken in order to determine the Tm of the protein.

### Example 4: Characterization of Type V Cas_3

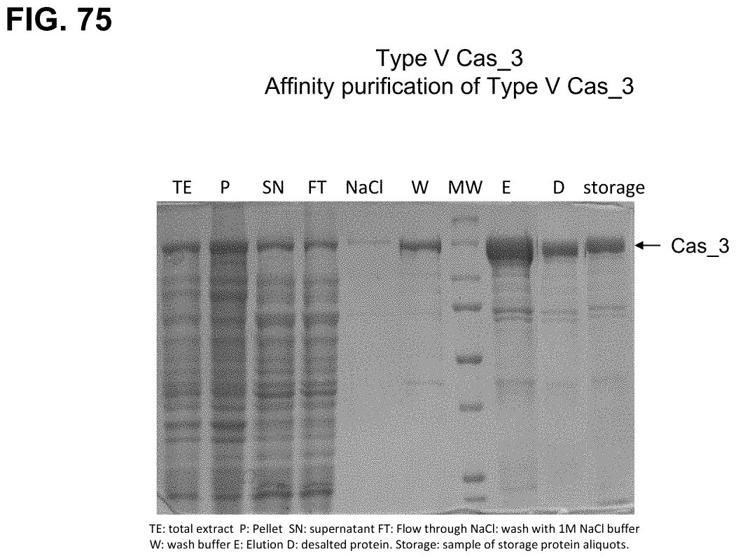

FIG. 75 shows a 10% SDS-PAGE analysis of Type V Cas_3 purification. TE: total extract (2 μl) P: Pellet (4 μl) SN: supernatant (4 μl) FT: Flow through (4 μl) NaCl: wash with E buffer (15 μl) F: wash with F buffer (15 μl) E: Elution with G buffer (8 μl) D: desalted protein (8 μl). Storage: sample of storage protein aliquots. Results are shown in FIG. 75.

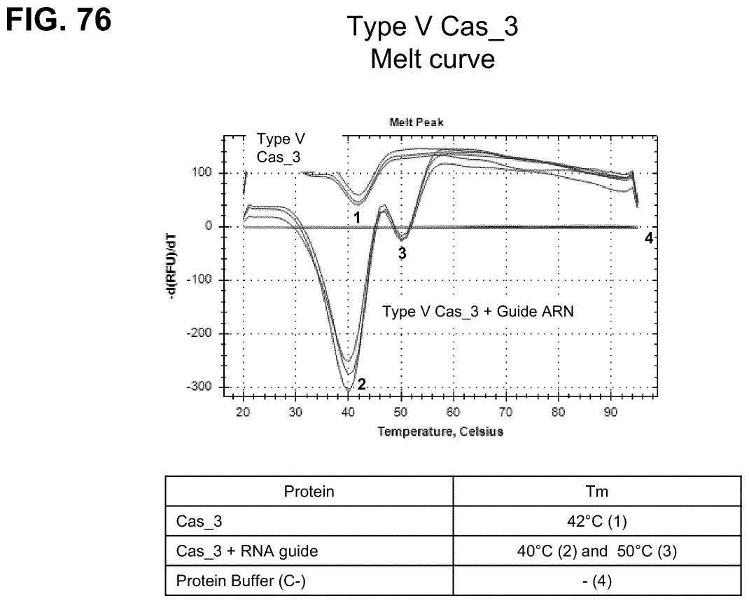

FIG. 76 shows the results of a temperature-based assay to assess the stability of Type V Cas_3 protein. The first derivative plots of the melting curve displaying the thermostability of apo protein form and its binary complex (Type V Cas_3+sgRNA). Melting curve was obtained by a thermal shift assay using Sypro Orange (Invitrogen). The first derivative plots of Type V Cas_3 and its binary complex are nearly overlapping [melting temperature (Tm)=40-42° C.], moreover a second peak appear at 50° C. in the binary complex evidencing two complexes in the reaction under this buffer conditions. All reactions were performed (apo protein or complex) to a final concentration of 1.8 μM in buffer (50 mM Tris-HCl pH 8, 200 mM NaCl, 20 mM MgCl₂, 1 mM DTT) with the addition of 10× SYPRO Orange (Invitrogen). Binary complex (protein+sgRNA) was formed at a 1:1 ratio. Apo and complexes were incubated at room temperature for 10 minutes prior to melting to assure complex formation. The reactions were then split into three 204, technical replicates. Protein melting assay was performed in a qPCR (Bio-Rad) over a temperature range from 20° C. to 95° C., at a rate of 1° C./minute, with 1 acquisitions/minute. The first derivative of the raw fluorescence data was taken in order to determine the Tm of the protein. Results are shown in FIG. 76.

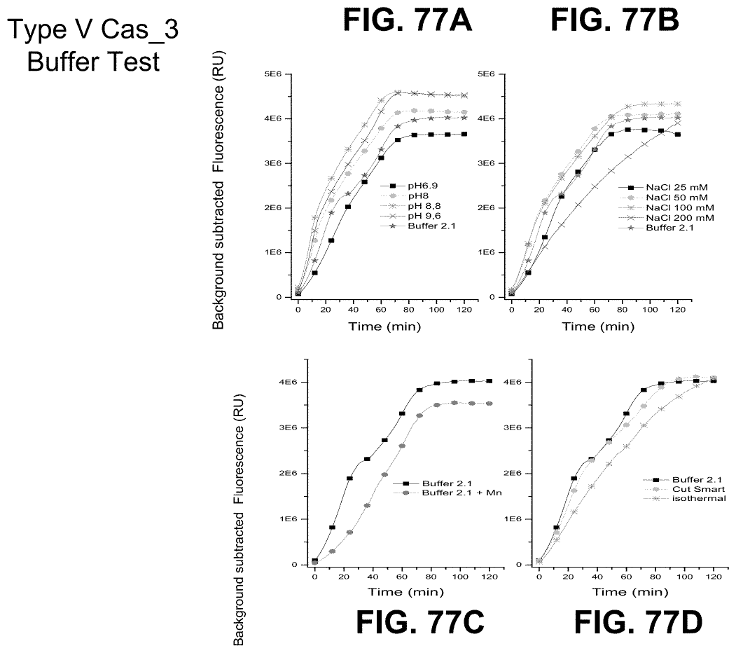

FIG. 77 shows ssDNA collateral cleavage of the Type V Cas_3 protein for an exemplary ssDNA Hantavirus target. A curve of pH (6.9 to 9.6), various salt concentration (25-200 mM NaCl), the addition of MnCl₂ and three commercial buffer conditions (2.1 NEB, CutSmart NEB and Isothermal Amplification Buffer NEB) were tested. The efficiency of trans-cleavage activity at different reaction buffer conditions was tested using customized ssDNA/56-FAM/TTATTATT/3IABkFQ/from IDT (Integrated DNA Technologies, Inc) as a reporter. The best activity was obtained in buffer 2.1 (New England Biotechnology), at high pH (>8) and low salt concentrations (25-100 mM). The addition of manganese (2 mM MnCl₂) to NEB 2.1 buffer does not improves the reaction.

Detection assay was performed at 30° C. using Type V Cas_3 complexes to a final concentration of 150 nM Type V Cas_3: 150 nM sgRNA: 10 nM activator in a solution containing 1× Binding Buffer and 625 nM of each ssDNA FAMQ reporter substrate in a 40 μl reaction. Three different commercial Binding Buffers were tested: NEB 2.1, CutSmart and Isothermal Amplification Buffer (New England Biotechnology), a curve of pH (from 6.8 to 9.6) was prepared using the base of a 2.1 NEB buffer (50 mM NaCl, 10 mM Tris-HCl, 10 mM MgCl₂, 100 g/ml BSA). The salt concentration curve (25-200 mM NaCl) was prepared at 7.9 pH from 2.1 NEB buffer (25-200 mM NaCl, 10 mM Tris-HCl, 10 mM MgCl₂, 100 g/ml BSA, pH 7.9). Reactions were incubated 120 minutes in a fluorescence plate reader Synergy H1 (Bio-Tek) and background-corrected fluorescence values were calculated by subtracting fluorescence values obtained from reactions carried out by triplicate in the absence of ssDNA Hanta target. Results are shown in FIG. 77

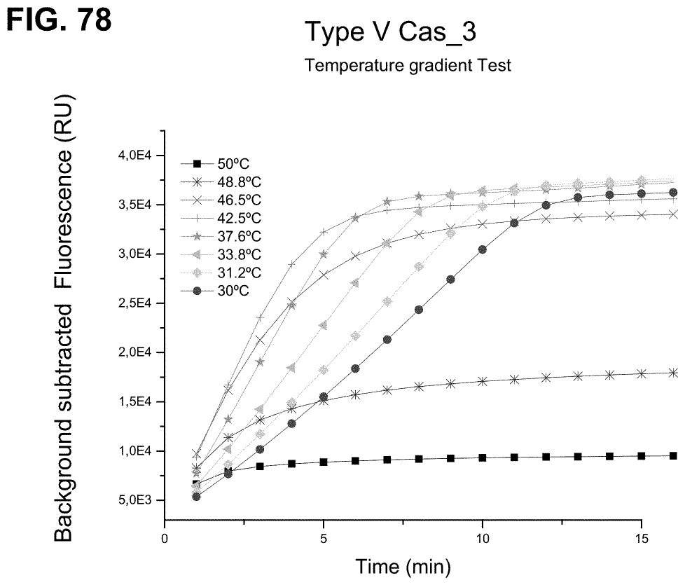

FIG. 78 shows the activity of Type V Cas_3 protein at different temperatures (30° C.-50° C.). The efficiency of trans-cleavage activity at different temperatures was tested using customized ssDNA/56-FAM/TTATTATT/3IABkFQ/from IDT (Integrated DNA Technologies, Inc) as a reporter. The results showed that Type V Cas_3 is able to cleave the ssDNA reporter in a wide range of temperatures from 30° C. to 46.5° C. showing a decrease in activity at higher temperatures (48-50° C.). The detection assay was performed from 30° C. to 50° C. using Type V Cas_3 complexes to a final concentration of 150 nM Cas: 150 nM sgRNA: 10 nM activator in a solution containing 1× Binding Buffer (50 mM NaCl, 10 mM Tris-HCl, 10 mM MgCl₂, 100 g/ml BSA, pH 7.9) and 625 nM of ssDNA FAMQ reporter substrate in a 40 μl reaction. Reactions were incubated in a qPCR (Bio-Rad) for 20 minutes with fluorescence measurements taken every 1 minute (ssDNA FQ substrates=λex: 485 nm; λem: 538 nm). Non-template negative control (NTC) fluorescence values were calculated from reactions carried out by triplicate in the absence of ssDNA Hanta target. Results are shown in FIG. 78.

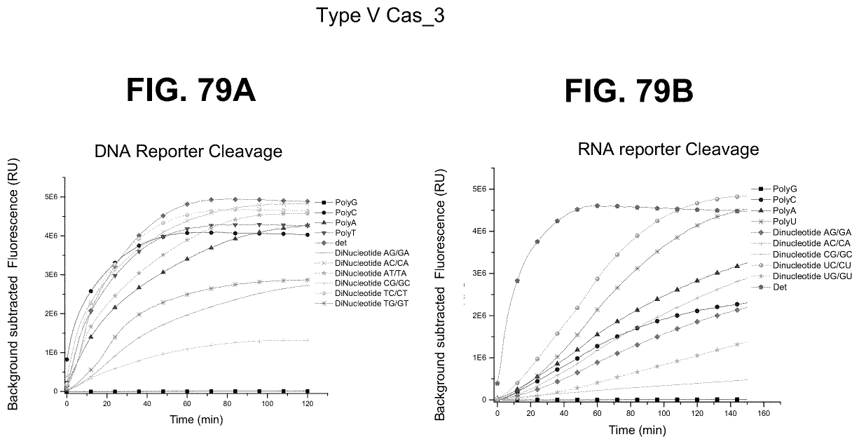

FIG. 79 shows Trans-cleavage activities on single-stranded reporters. The specificity of trans-cleavage activity using customized ssDNA or ssRNA as reporters was tested. The results showed that Type V Cas_3 protein is able to cleave DNA or RNA reporters with different specificities. Both DNA and RNA guanine homopolymers (Poly G) reporters were not cleaved by Type V Cas_3 protein and as a consequence a decreased activity was observed in dimers that contained guanine nucleotides in their composition. Detection assays were performed at 40° C. using Type V Cas_3 complex to a final concentration of 150 nM Type V Cas_3: 150 nM sgRNA: 10 nM activator in a solution containing 1× Binding Buffer (50 mM NaCl, 10 mM Tris-HCl, 10 mM MgCl₂, 100 g/ml BSA, pH 7.9) and 625 nM of FAMQ reporter substrates in a 40 μl reaction. Reactions were incubated in a fluorescence plate reader Synergy H1 (Bio-Tek) and background-corrected fluorescence values were calculated by subtracting fluorescence values obtained from reactions carried out by triplicate in the absence of Hanta target.

### Example 5: Characterization of Type V Cas_4

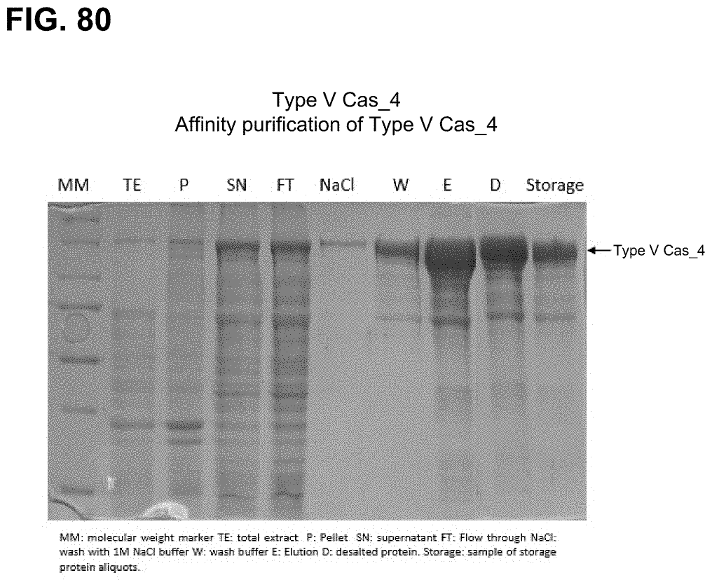

FIG. 80 shows a 10% SDS-PAGE analysis of Type V Cas_4 purification. The Type V Cas_4 protein was purified as recombinant protein expressed in E. coli NiCo21 (DE3) cells (NEB #C2529H) harboring the pET28a/Type V Cas_4-H6X expression plasmid by growing in LB broth culture medium at 37° C. followed by induction of expression overnight at 18° C. in presence of 0.25 mM IPTG. Cells were disrupted by sonication prior to chromatographic purification. Recombinant protein was purified using a His-Trap HP (Ni-NTA GE Healthcare) followed by a HiPrep™ 26/10 desalting column (GE Healthcare) where the protein was desalted into storage buffer containing Tris-HCl 50 mM (pH 8), NaCl 200 mM, MgCl₂ 20 mM, DTT 1 mM. Protein purity was controlled by Coomassie blue staining after SDS-PAGE on a 10% polyacrylamide gel. Protein concentrations were determined by UV spectroscopy and Qubit protein assay (Invitrogen). Purified proteins were stored at −80° C.

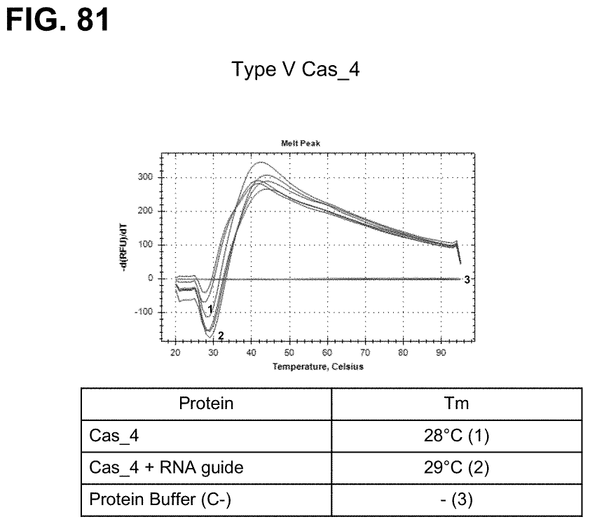

FIG. 81 shows the results of a temperature-based assay to assess the stability of Type V Cas_4 protein. The first derivative plots of the melting curve displaying the thermostability of apo protein form and its binary complex (Type V Cas_4+sgRNA). Melting curve was obtained by a thermal shift assay using Sypro Orange (Invitrogen). The first derivative plots of Type V Cas_4 and its binary complex are nearly overlapping [melting temperature (Tm)=28-29° C.]. All reactions were performed (apo protein or complex) to a final concentration of 1.8 μM in buffer (50 mM Tris-HCl pH 8, 200 mM NaCl, 20 mM MgCl₂, 1 mM DTT) with the addition of 10× SYPRO Orange (Invitrogen). Binary complex (protein+sgRNA) was formed at a 1:1 ratio. Apo and complexes were incubated at room temperature for 10 minutes prior to melting to assure complex formation. The reactions were then split into three 204, technical replicates. Protein melting assay was performed in a qPCR (Bio-Rad) over a temperature range from 20° C. to 95° C., at a rate of 1° C./minute, with 1 acquisitions/minute. The first derivative of the raw fluorescence data was taken in order to determine the Tm of the protein. Results are shown in FIG. 81.

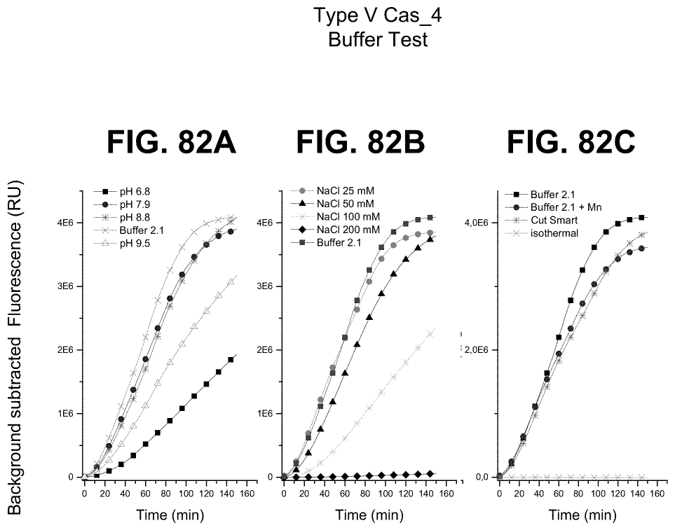

FIG. 82A-82C Activity test in different reaction buffer conditions. FIGS. 82A-82C shows ssDNA collateral cleavage of the Type V Cas_4 protein for an exemplary ssDNA Hantavirus target. A curve of pH (6.8 to 9.5), various salt concentration (25-200 mM NaCl), the addition of MnCl₂ and three commercial buffer conditions (2.1 NEB, CutSmart NEB and Isothermal Amplification Buffer NEB) were tested. The efficiency of trans-cleavage activity at different reaction buffer conditions was tested using customized ssDNA/56-FAM/TTATTATT/3IABkFQ/from IDT (Integrated DNA Technologies, Inc) as a reporter. The best activity was obtained in buffer 2.1 (New England Biotechnology), at pH between 7.9 and 8.8. High salt concentrations (100-200 mM) were detrimental for Type V Cas_4protein activity. The addition of manganese (2 mM MnCl₂) to NEB 2.1 buffer does not improves the reaction.

Detection assay was performed at 30° C. using Type V Cas_4 complexes to a final concentration of 150 nM Type V Cas_4: 150 nM sgRNA: 10 nM activator in a solution containing 1× Binding Buffer and 625 nM of each ssDNA FAMQ reporter substrate in a 40 μl reaction. Three different commercial Binding Buffers were tested: NEB 2.1, CutSmart and Isothermal Amplification Buffer (New England Biotechnology), a curve of pH (from 6.8 to 9.5) was prepared using the base of a 2.1 NEB buffer (50 mM NaCl, 10 mM Tris-HCl, 10 mM MgCl₂, 100 g/ml BSA). The salt concentration curve (25-200 mM NaCl) was prepared at 7.9 pH from 2.1 NEB buffer (25-200 mM NaCl, 10 mM Tris-HCl, 10 mM MgCl₂, 100 g/ml BSA, pH 7.9). Reactions were incubated 150 minutes in a fluorescence plate reader Synergy HI (Bio-Tek) and background-corrected fluorescence values were calculated by subtracting fluorescence values obtained from reactions carried out by triplicate in the absence of ssDNA Hanta target.

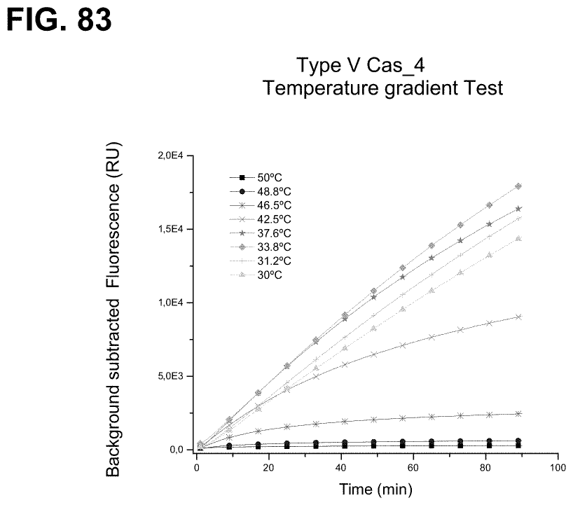

FIG. 83 shows the activity of Type V Cas_4 protein at different temperatures (30° C.-50° C.). The efficiency of trans-cleavage activity at different temperatures was tested using customized ssDNA/56-FAM/TTATTATT/3IABkFQ/from IDT (Integrated DNA Technologies, Inc) as a reporter. The results showed that Type V Cas_4 is able to cleave the ssDNA reporter in a wide range of temperatures from 30° C. to 37.6° C. showing a decrease in activity at higher temperatures (>42.5° C.). The detection assay was performed from 30° C. to 50° C. using Type V Cas_4 complexes to a final concentration of 150 nM Cas: 150 nM sgRNA: 10 nM activator in a solution containing 1× Binding Buffer (50 mM NaCl, 10 mM Tris-HCl, 10 mM MgCl₂, 100 g/ml BSA, pH 7.9) and 625 nM of ssDNA FAMQ reporter substrate in a 40 μl reaction. Reactions were incubated in a qPCR (Bio-Rad) for 20 minutes with fluorescence measurements taken every 1 minute and plotted every 8 minutes (ssDNA FQ substrates=λex: 485 nm; λem: 538 nm). Non-template negative control (NTC) fluorescence values were calculated from reactions carried out by triplicate in the absence of ssDNA Hanta target. Results are shown in FIG. 83

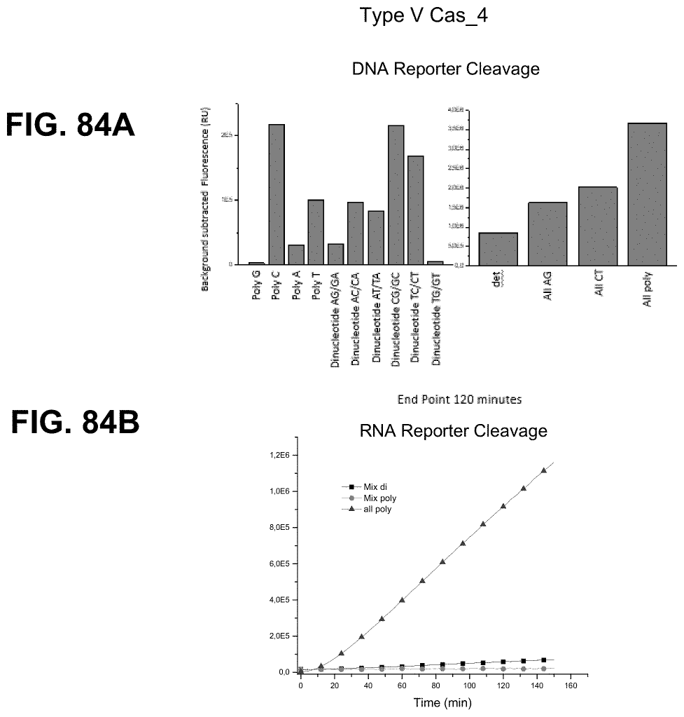

FIGS. 84A-84B shows trans-cleavage activities on single-stranded reporters. We tested the specificity of trans-cleavage activity using customized ssDNA or ssRNA as reporters. The results showed that Type V Cas_4 protein is able to cleave DNA reporters with different specificities but not the RNA reporters tested. Moreover, DNA guanine homopolymers (Poly G) reporter were not cleaved by Type V Cas_4 protein while DNA cytokine homopolymer (Poly C) reporter and their respective dimeric variants showed the best cleavage values. Detection assays were performed at 35° C. using Type V Cas_4 complex to a final concentration of 150 nM Type V Cas_3 1: 150 nM sgRNA: 10 nM activator in a solution containing 1× Binding Buffer (50 mM NaCl, 10 mM Tris-HCl, 10 mM MgCl₂, 100 g/ml BSA, pH 7.9) and 625 nM of FAMQ reporter substrates in a 40 μl reaction. Reactions were incubated in a fluorescence plate reader Synergy H1 (Bio-Tek) and background-corrected fluorescence values were calculated by subtracting fluorescence values obtained from reactions carried out by triplicate in the absence of Hanta target. Results are shown in FIGS. 84A-84B.

### Example 6: Characterization of Type V Cas_5

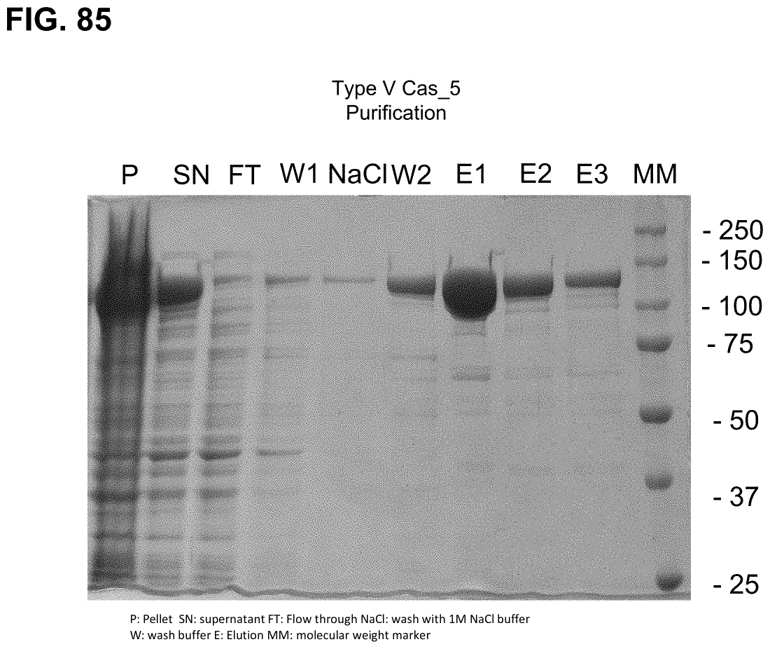

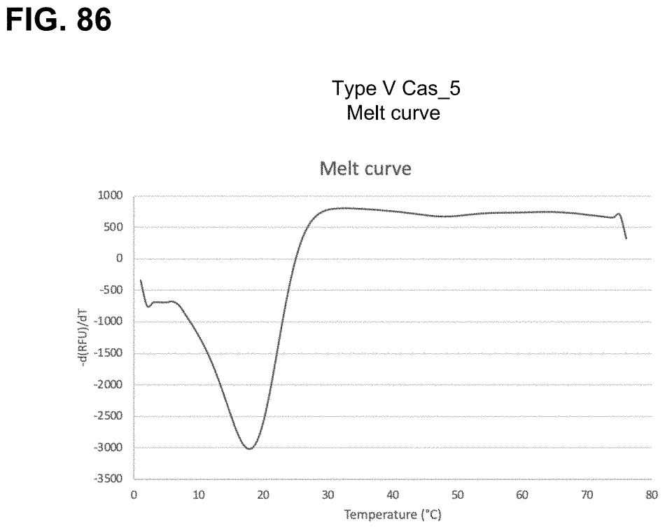

FIG. 85 shows Type V Cas_5 purification and FIG. 86 shows thermal shift analysis. Type V Cas_5 protein was purified using Ni-NTA agarose chromatography. The thermal stability of the purified protein was tested using SYPRO® Orange Protein Gel Stain (Merck) as denaturalization reporter. The melting curve observed indicates that the protein is stable up to 36° C. in absence of scout and sgRNA. Type V Cas_5 protein coding sequence was codon-optimized and synthesized by GeneScript and then cloned into pET28a (Novagen) with N-terminal 6×His tagging (SEQ ID NO: 146). Expression plasmids were transformed into E. coli NiCo21 (DE3) (NEB). For protein expression, cells were grown with shaking at 200 rpm and 37° C. until the OD 600 reached 0.68, and IPTG was then added to a final concentration of 0.25 mM followed by further culture of the cells at 28° C. for about 6 h before the cell harvesting. Cells were resuspended in 10 mL of buffer A (50 mM Tris-HCl pH 8.0, 0.5 M NaCl, 1 mM DTT and 10% glycerol) with protease inhibitor cocktail (Promega), 10 mM imidazole and 0.1 mg/ml lysozyme. After a 15 min incubation at 37° C., cells were lysed by sonication for 10 minutes with 10 s on and 10 s off cycle. Cell debris and insoluble particles were removed by centrifugation (15,000 rpm for 40 min). After centrifuging, the supernatant was loaded onto a 1 mL Crude His-Trap column (GE Healthcare) equilibrated in buffer A with 10 mM imidazol on an AKTA Pure 25L device (GE Healthcare Life Sciences). The elution was performed by a step gradient of buffer B (buffer A plus 120 mM imidazole). The elution was dialyzed with dialysis buffer (50 mM Tris-HCl pH 8.0, 200 mM NaCl, 1 mM DTT and 20 mM MgCl₂). Results are shown in FIG. 85

Thermal stability assay was performed at a temperature range from 20° C. to 90° C. using 15 ug of Type V Cas_5 protein in a solution containing 1× Desalting buffer desalting buffer (50 mM Tris-HCl pH 8, 200 mM NaCl, 20 mM MgCl2, 1 mM DTT) and 10× of SYPRO® dye in a 30 μl reaction. The mix was incubated in a qPCR (Bio-Rad) increasing the temperature from 20° C. to 90° C. with fluorescence measurements taken every 1° C. (SYPRO® dye=λex: 300 nm; λem: 570 nm). A no-protein negative control fluorescence values were calculated from samples without protein. Results are shown in FIG. 86.

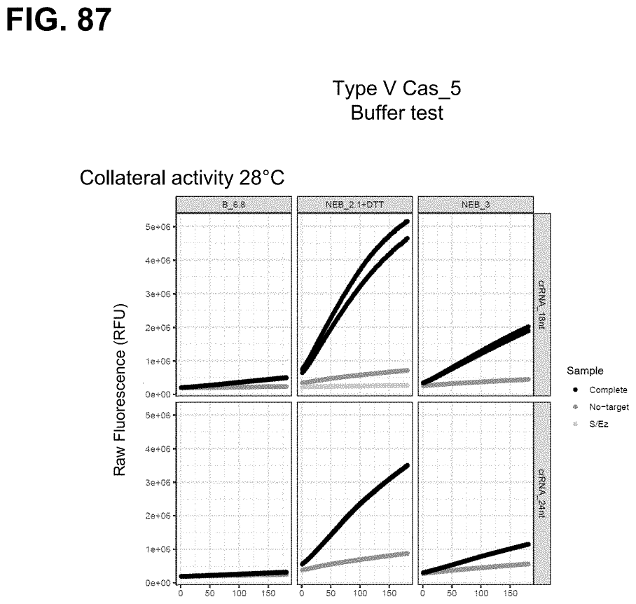

FIG. 87 shows trans-cleavage activity testing using two different sgRNA and three buffer conditions. The efficiency of trans-cleavage activity on each condition was tested using customized ssDNA/56-FAM/TTATTATT/3IABkFQ/and ssDNA/56-F/3IABkFQ/from IDT (Integrated DNA Technologies, Inc.) as a reporter. 18 nucleotides sgRNA presents higher activity than 24 nucleotides sgRNA. The best activity was observed when in NEB 2.1 supplemented with 1 mM DTT. Detection assay was performed at 28° C. using Type V Cas_5 complexes to a final concentration of 250 nM Type V Cas_5: 250 nM scoutRNA: 250 nM sgRNA: 50 nM activator in a solution containing 1× Binding Buffer and 625 nM of each ssDNA FAMQ reporter substrate in a 40 μl reaction. Three different Binding Buffers were tested: B_6.8 (50 mM Tris pH 6.8, 100 mM NaCl, 10 mM MgCl, 1 mM DTT), NEB 2.1+DTT (50 mM NaCl, 10 mM Tris-HCl, 10 mM MgCl2, 100 ug/ml BSA, pH 7.9, 1 mM DTT) and NEB 3.0 (100 mM NaCl, 50 mM Tris-HCl, 10 mM MgCl2, pH 7.9, 1 mM DTT). A no-enzyme control was added using the 18 nucleotides sgRNA in NEB 2.1+DTT buffer. Reactions were incubated in a fluorescence plate reader Synergy H1 (Bio-Tek) for 180 minutes with fluorescence measurements taken every 1 minute (ssDNA FQ substrates=λex: 485 nm; λem: 538 nm). Non-template negative control (NTC) fluorescence values were calculated from reactions carried out in the absence of ssDNA Hanta target. Results are depicted in FIG. 87.

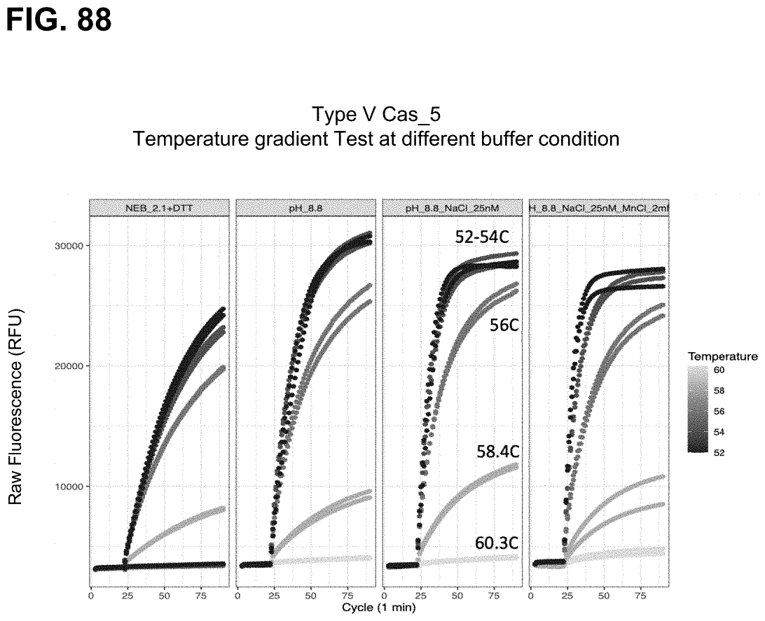

FIG. 88 shows the activity of Type V Cas_5 protein in a temperature curve (52° C.-60° C.) and three buffer conditions. The enzyme was incubated 20 minutes at the reported temperatures before activation with ssDNA Hanta target. The efficiency of trans-cleavage activity on each condition was tested using customized FAM/TTATTATT/3IABkFQ/and ssDNA/56-FAM/3IABkFQ/from IDT (Integrated DNA Technologies, Inc.) as a reporter. The results showed that Type V Cas_5 is able to cleave with good efficiency the ssDNA reporters between 52° C. and 56° C. The best activity was observed in buffer with pH 8.8 and 25 mM NaCl. Detection assay was performed at 52° C., 54° C., 56° C., 58.4° C. and 60.3° C. using Type V Cas_5 complexes to a final concentration of 125 nM Type V Cas_5: 125 nM scoutRNA: 125 nM sgRNA: 25 nM activator in a solution containing 1× Binding Buffer and 625 nM of each ssDNA FAMQ reporter substrate in a 40 μl reaction. Three different Binding Buffers were tested: NEB 2.1+DTT (Tris 10 mM pH 7.9/NaCl 50 mM/MgCl 10 mM/BSA 100 ug/mL/DTT 1 mM), pH 8.8 (Tris 10 mM pH 8.8/NaCl 50 mM/MgCl 10 mM/BSA 100 ug/mL/DTT 1 mM) and pH 8.8 NaC1 25 nM MnCl 2 nM (Tris 10 mM pH 8.8/NaCl 25 mM/MgCl 10 mM/BSA 100 ug/mL/DTT 1 mM/MnCl 2 nM). Reactions were incubated in a qPCR (Bio-Rad) for 60 minutes with fluorescence measurements taken every 1 minute (ssDNA FQ substrates=λex: 485 nm; λem: 538 nm). Non-template negative control fluorescence values were calculated from reactions carried out in the absence of ssDNA Hanta target. Results are shown in FIG. 88.

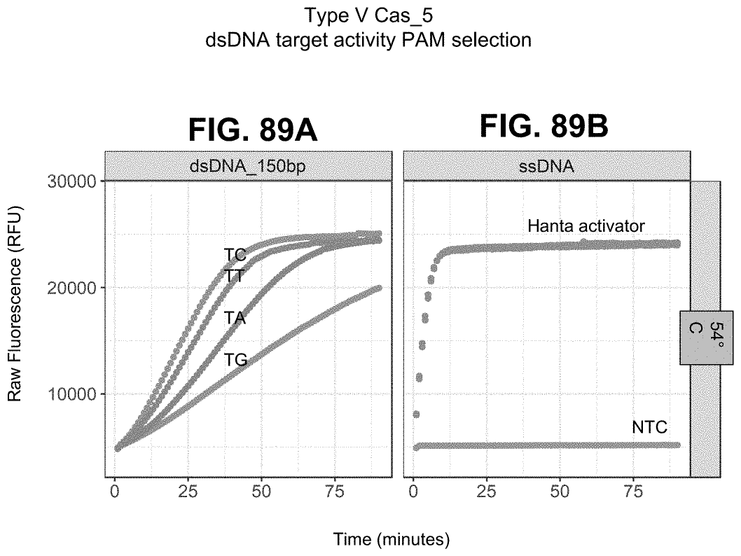

FIGS. 89A-89B are a PAM selectivity test. The Type V Cas_5 activation on different left-PAM sequences was tested using short dsDNA molecules (146 bp) as targets and customized/56-FAM/TTATTATT/3IABkFQ/and ssDNA/56-FA/3IABkFQ/from IDT (Integrated DNA Technologies, Inc.) as reporters respectively. The results showed that Type V Cas_5 is activated whit more efficiency when TC or TT PAM sequences. TA PAM sequence target present a reduce activity compared to TC or TT and the less activity is observed with TG PAM sequence. Detection assay was performed at 54° C. Type V Cas_5 complexes to a final concentration of 125 nM Type V Cas_5: 125 nM scoutRNA: 125 nM sgRNA: 10 nM target in a solution containing 1× Binding Buffer (Tris 10 mM pH 8.8/NaCl 25 mM/MgCl 10 mM/MnCl 2 mM/BSA 100 ug/mL/DTT 1 mM) and 625 nM of each ssDNA FAMQ reporter substrate in a 40 μl reaction. Reactions were incubated in a qPCR (Bio-Rad) for 90 minutes with fluorescence measurements taken every 1 minute (ssDNA FQ substrates=λex: 485 nm; λem: 538 nm). Non-template negative control fluorescence values were calculated from reactions carried out in the absence of ssDNA Hanta target. ssDNA Hanta target was used as positive control. Results are shown in FIGS. 89A-89B.

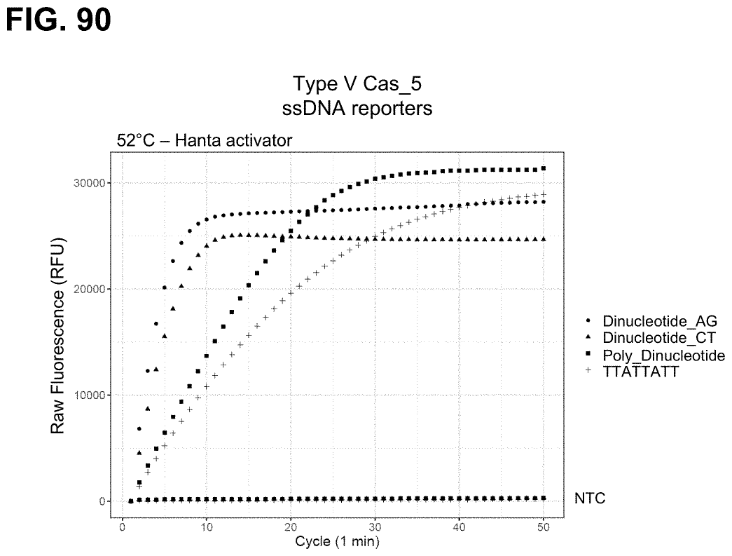

FIG. 90 shows the results of the differential efficiency in dinucleotide single-stranded reporter cleavage. Different dinucleotide reporter sequences were tested showing a significant increase in Type V Cas_5 activity. This enzyme has demonstrated a highly efficiency in All Dinucleotide A-G cleavage, evidenced by increased fluorescence in compare with ssDNA determined FAMQ TTATTATT reporter sequence. Detection assay was performed at 52° C. Type V Cas_5 complexes to a final concentration of 125 nM Type V Cas_5: 125 nM scoutRNA: 125 nM sgRNA: 10 nM ssDNA Hanta target in a solution containing 1× Binding Buffer (Tris 10 mM pH 8.8, NaCl 25 mM, MgCl₂ 10 mM, MnCl₂ 2 mM, BSA 100 ug/mL and DTT 1 mM) and 625 nM of customized FAMQ reporter substrates (/56-FAM/TTATTATT/3IABkFQ/, All Dinucleotide_A-G/56 FAM/ATACAGAGTGCG/3IABkFQ/(SEQ ID NO: 143), All Dinucleotide CT/56-FAM/TATGTCTCACGC/3IABkFQ/(SEQ ID NO: 144) and All Polynucleotides/56-FAM/AAATTTCCCGGG/3IABkFQ/(SEQ ID NO: 145) (12 nt) from IDT (Integrated DNA Technologies, Inc.)) in a 40 μl reaction. Reactions were incubated in a qPCR (Bio-Rad) for 50 minutes with fluorescence measurements taken every 1 minute (ssDNA FQ substrates=λex: 485 nm; λem: 538 nm). Non-template negative control fluorescence values were calculated from reactions carried out in the absence of ssDNA Hanta target. Results are shown in FIG. 90.

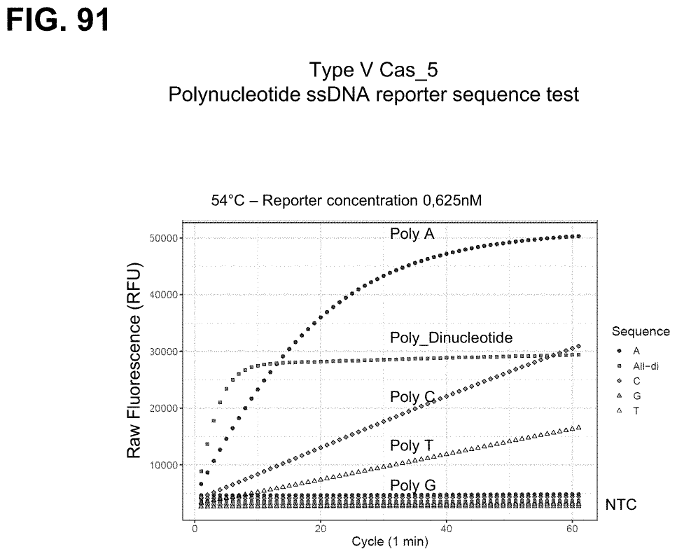

FIG. 91 shows the results from a differential efficiency in single-base DNA reporter cleavage. Different reporters with only one base in their sequences were tested in Type V Cas_5 activity. This enzyme has demonstrated that single base reporter sequences are cleaved with less efficiency that mixed bases reporter sequences. Among single base reporters, poly-A is cleaved with the highest efficiency followed by poly-C and poly-T. No cleavage was observed in Poly-G reporter. Detection assay was performed at 54° C. Type V Cas_5 complexes to a final concentration of 125 nM Type V Cas_5: 125 nM scoutRNA: 125 nM sgRNA: 10 nM ssDNA Hanta target in a solution containing 1× Binding Buffer (Tris 10 mM pH 8.8, NaCl 25 mM, MgCl₂ 10 mM, MnCl₂ 2 mM, BSA 100 ug/mL, DTT 1 mM) and 625 nM of customized FAMQ reporter substrates (All Polynucleotides/56-FAM/AAATTTCCCGGG/3IABkFQ/(SEQ ID NO: 145) (12 nt), Poly C/56-FAM/CCCCCCC/3IABkFQ/, Poly A/56-FAM/AAAAAAA/3IABkFQ/, Poly T/56-FAM/TTTTTTT/3IABkFQ/and Poly G/56-FAM/GGGGGG/3IABkFQ/from IDT (Integrated DNA Technologies, Inc.)) in a 40 μl reaction. Reactions were incubated in a qPCR (Bio-Rad) for 60 minutes with fluorescence measurements taken every 1 minute (ssDNA FQ substrates=λex: 485 nm; λem: 538 nm). Non-template negative control fluorescence values were calculated from reactions carried out in the absence of ssDNA Hanta target. Results are shown in FIG. 91.

### Example 7: Characterization of Type VI Cas_2

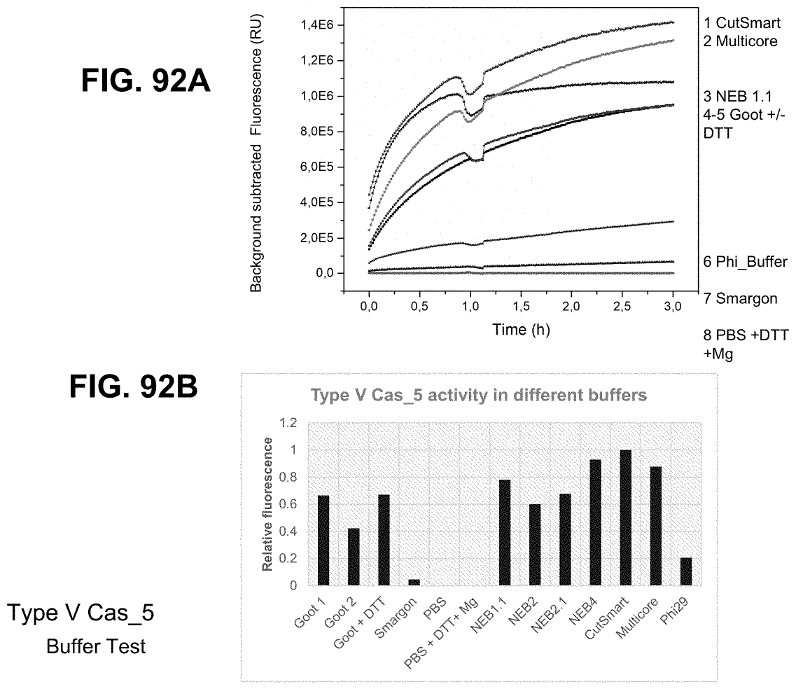

FIGS. 92A-92B shows the results of the collateral activity of Type VI Cas_2 protein complex in different buffer solutions. The efficiency of trans-cleavage activity of Type VI Cas_2 protein was tested in different buffer solutions using customized ssRNA/56-FAM/rUrUrUrUrUrUrU/3IABkFQ/from IDT (Integrated DNA Technologies, Inc.) as a reporter. FIG. 92A. Shows the time course cleavage over 3 h in: 1. CutSmart buffer from NEB (50 mM Potassium Acetate, 20 mM Tris-acetate, 10 mM Magnesium Acetate, 100 μg/ml BSA, pH 7.9); 2. Multicore buffer from Promega (25 mM Tris-acetate, 100 mM Potassium Acetate, 10 mM Magnesium Acetate, 1 mM DTT, pH 7.5); 3. NEB 1.1 buffer from NEB (10 mM Bis-Tris-Propane-HCl, 10 mM MgCl₂, 100 μg/ml BSA, pH 7); 4. Goot 1 buffer (20 mM HEPES, 60 mM NaCl, 6 mM MgCl₂, pH 6.8); 5. Goot 1 buffer supplemented with 2 mM DTT; 6. Phi buffer from NEB (50 mM Tris-HCl, 10 mM MgCl₂, 10 mM (NH₄)₂SO₄, 4 mM DTT, pH 7.5); 7. Smargon buffer (10 mM Tris-HCl, 50 mM NaCl, 0.5 mM MgCl₂, 0.1% BSA, pH 7.5); 8. PBS buffer (137 mM NaCl, 2.7 mM KCl, 8 mM Na2HPO4, 2 mM KH2PO4, pH 7.4); 9. PBS buffer supplemented with 1 mM DTT and 10 mM MgCl₂. FIG. 92B. Shows the endpoint activity relative to CutSmart buffer after 180 min in different buffer solutions: Goot 1 buffer; Goot 2 buffer (40 mM Tris-HCl, 60 mM NaCl, 6 mM MgCl2, pH 7.3); Goot 1 buffer supplemented with 2 mM DTT; Smargon buffer; PBS buffer; PBS buffer supplemented with 1 mM DTT and 10 mM MgCl₂; NEB 2 buffer from NEB (10 mM Tris-HCl, 50 mM NaCl, 10 mM MgCl2, 1 mM DTT, pH 7.9); NEB 2.1 buffer from NEB (10 mM Tris-HCl, 50 mM NaCl, 10 mM MgCl2, 100 μg/ml BSA, pH 7.9); NEB 4 buffer from NEB (50 mM Potassium Acetate, 20 mM Tris-acetate, 10 mM Magnesium Acetate, 1 mM DTT, pH 7.9); CutSmart buffer; Multicore buffer and Phi buffer. Reaction in CutSmart buffer demonstrated the best activity, evidenced for the highest fluorescence values. The protein also showed high activity values in NEB 4 and Multicore buffers which share similar composition to CutSmart buffer. The reaction was initiated by preparing complexes to a final concentration of 150 nM Type VI Cas_2: 75 nM sgRNA: 20 nM activator (31 nt. ssRNA from Synthego) and 150 nM of ssRNA FAMQ reporter substrate in a 40 μl reaction, in each of the aforementioned buffer solutions at 37° C. Reactions were incubated in a Synergy H1 microplate reader (Bio-Tek) for 180 minutes with fluorescence measurements taken every 2 minutes (ssRNA FQ substrates=λex: 485 nm; λem: 538 nm). Non-template negative control (NTC) fluorescence values were calculated from reactions carried out in the absence of ssRNA Hanta target.

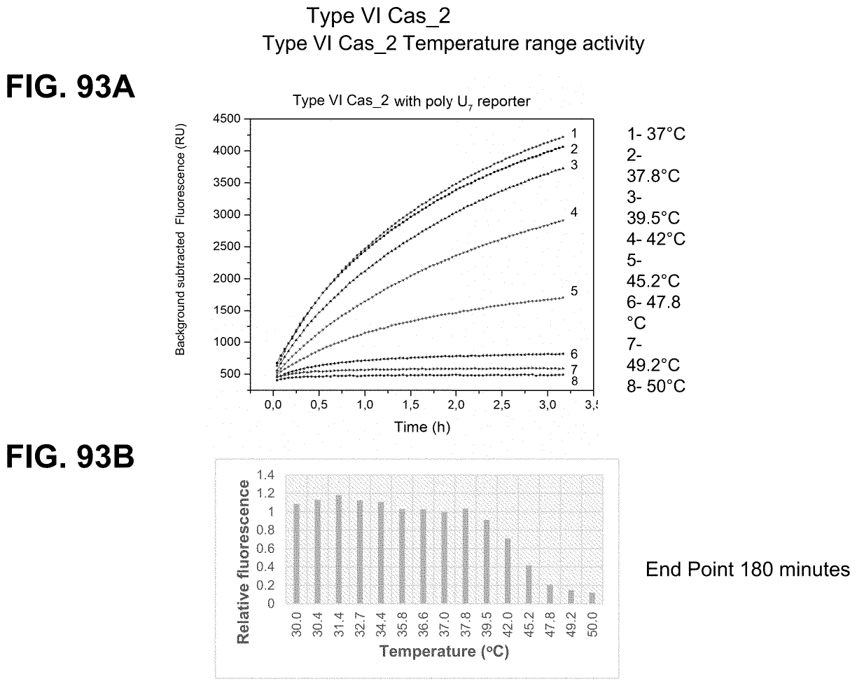

FIGS. 93A-93B shows collateral activity of the Type VI Cas_2 protein complex in a temperature curve (30° C.-50° C.). The efficiency of trans-cleavage activity at different temperatures was tested using customized ssRNA/56-FAM/rUrUrUrUrUrUrU/3IABkFQ/from IDT (Integrated DNA Technologies, Inc.) as a reporter. The temperatures analyzed over time (FIG. 93A) were: 37.0° C., 37.8° C., 39.5° C., 42° C., 45.2° C., 47.8° C., 49.2° C. and 50°. The temperatures analyzed as endpoint after 180 min (FIG. 93B) included 30.0° C., 30.4° C., 31.4° C., 32.7° C., 34.4° C., 35.8° C., 36.6° C., 37.0° C., 37.8° C., 39.5° C., 42° C., 45.2° C., 47.8° C., 49.2° C. and 50° and were expressed relative to 37° C. The results showed that Type VI Cas_2 was able to cleave the ssRNA reporter efficiently between 30° C. and 42° C., with an optimal activity at 31.4° C. Detection assay was performed at the different temperatures using Type VI Cas_2 complexes to a final concentration of 150 nM Type VI Cas_2: 75 nM sgRNA: 20 nM activator (31 nt. ssRNA from Synthego) in a solution containing 1× Binding Buffer (50 mM Potassium Acetate, 20 mM Tris-acetate, 10 mM Magnesium Acetate, 100 μg/ml BSA, pH 7.9) and 150 nM of ssRNA FAMQ reporter substrate in a 40 μl reaction. Reactions were incubated in a qPCR (Bio-Rad) for 180 minutes with fluorescence measurements taken every 2.5 minutes (ssRNA FQ substrates=λex: 485 nm; λem: 538 nm). Non-template negative control (NTC) fluorescence values were calculated from reactions carried out in the absence of ssRNA Hanta target.

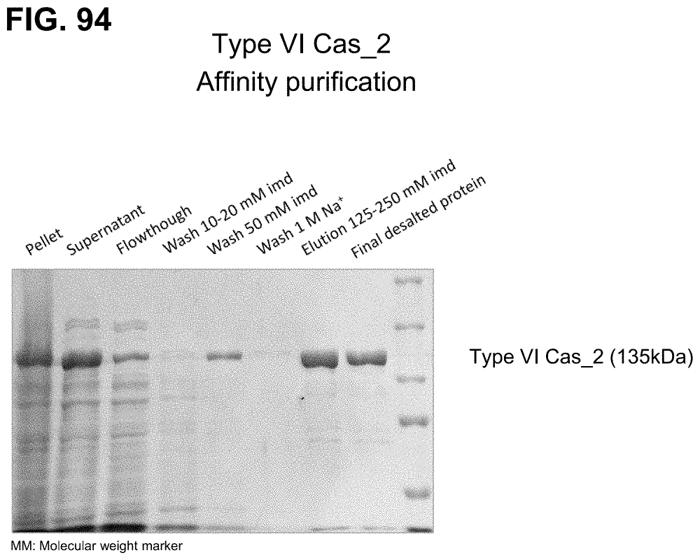

FIG. 94 shows the results of a 10% SDS-PAGE analysis of Type VI Cas_2 purification. The Type VI Cas_2 protein was purified as recombinant protein expressed in E. coli Rosetta (DE3) cells (Merck #70954) harboring the pET28a/Type VI Cas_2-H6X expression plasmid by growing in LB broth culture medium at 37° C. followed by induction of 6 hs expression at 20° C. in presence of 0.25 mM IPTG. Cells were disrupted by sonication prior to chromatographic purification. Recombinant protein was purified using a His-Trap HP (Ni-NTA GE Healthcare) followed by a HiPrep™ 26/10 desalting column (GE Healthcare) where the protein was desalted into storage buffer containing 10 mM HEPES, 500 mM NaCl, 1 mM DTT, pH 7.5. Protein purity was controlled by Coomassie blue staining after SDS-PAGE on a 10% polyacrylamide gel. Protein concentrations were determined by UV spectroscopy and Qubit protein assay (Invitrogen). Purified proteins were stored at −80° C.

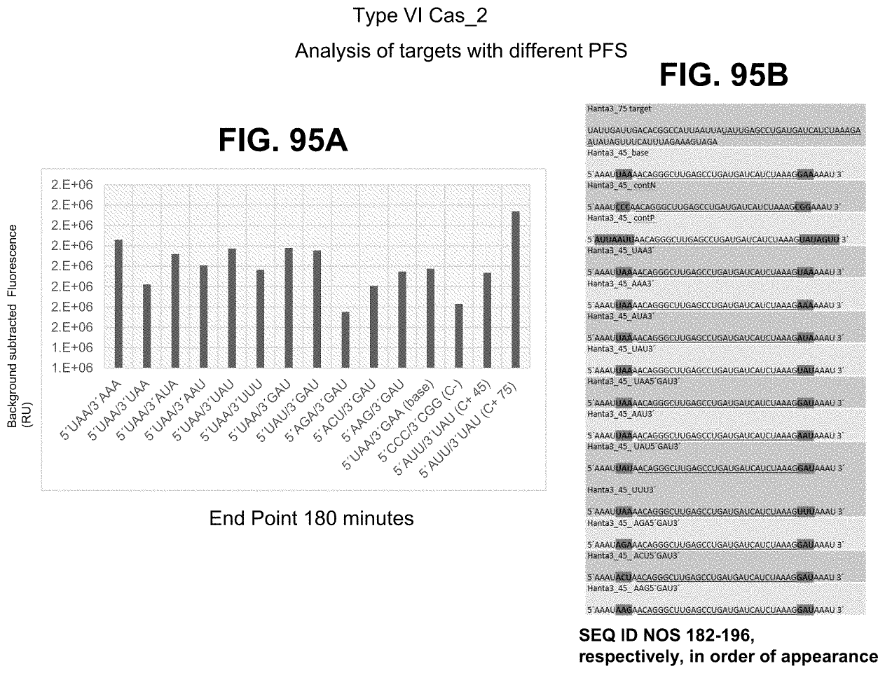

FIGS. 95A-95B Collateral activity of the Type VI Cas_2 protein complex for a ssRNA target with variable protospacer flanking sequences (PFS). The efficiency of the Type VI Cas_2 protein complex to cleave targets with different PFS was analyzed indirectly through the trans-cleavage activity of the enzyme, using customized ssRNA/56-FAM/rUrUrArUrUrArUrU/3IABkFQ/from IDT (Integrated DNA Technologies, Inc.) as a reporter. The different PFS present in the target comprised the 5′ sequences: AAAUUAA, AAAUCCC, AAAUUAU, AAAUAGA, AAAUACU, AAAUAAG, AUUAAUU and 3′ sequences: GAAAAAU, CGGAAAU, UAAAAAU, AAAAAAU, AUAAAAU, UAUAAAU, GAUAAAU, AAUAAAU, UUUAAAU, UAUAGUU. The results showed that Type VI Cas_2 was able to cleave all the targets tested with similar efficiency. The target with flanking sequence 5″AAAUAGA and 3′ GAUAAAU reported the lowest fluorescence value followed by the target with flanking sequence 5′ AAAUCCC and 3′ CGGAAAU. For the same flanking sequences (5′ AUUAAUU and 3′ UAUAGUU), the 75-nt. target displayed higher fluorescence than the 45-nt. target. Experiments were performed in 40 μL reaction volume containing 1× Binding Buffer (50 mM Potassium Acetate, 20 mM Tris-acetate, 10 mM Magnesium Acetate, 100 μg/ml BSA, pH 7.9), Type VI Cas_2 protein complexed to a final concentration of 100 nM Type VI Cas_2: 50 nM sgRNA: 20 nM of each of the aforementioned activators and 150 nM of ssRNA FAMQ reporter substrate. Reactions were incubated at 30° C. in a Synergy H1 microplate reader (Bio-Tek) for 180 minutes as the endpoint time (ssRNA FQ substrates=λex: 485 nm; λem: 538 nm). Non-template negative control (NTC) fluorescence values were calculated from reactions carried out in the absence of ssRNA Hanta target. Results are shown in FIGS. 95A-95B.

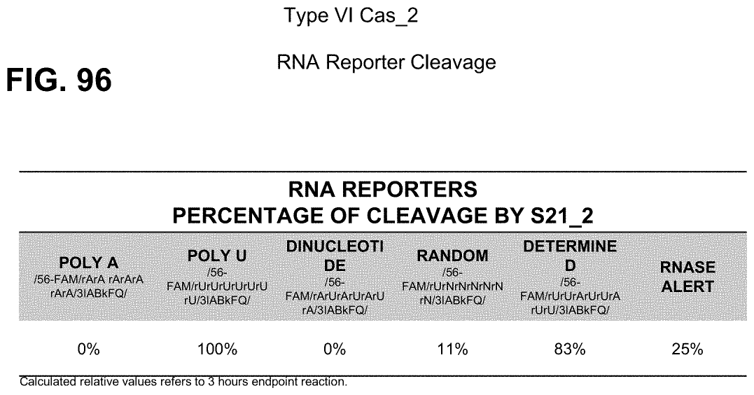

FIG. 96 shows the collateral activity of the Type VI Cas_2 protein complex for different customized ssRNA reporter substrates. Efficiency of trans-cleavage activity for different customized ssRNA reporters from IDT (Integrated DNA Technologies, Inc.). The ssRNA reporters analyzed were: poly A (/56-FAM/rArArArArArArA/3IABkFQ/), poly U (/56-FAM/rUrUrUrUrUrUrU/3IABkFQ/), dinucleotide (/56-FAM/rArUrArUrArUrA/3IABkFQ/), random (/56-FAM/rUrNrNrNrNrNrN/3IABkFQ/), determined (/56-FAM/rUrUrArUrUrArUrU/3IABkFQ/) and RNaseAlert™ substrate from IDT. Fluorescence values were expressed relative to the highest fluorescence value reached in the experiment. The results showed that Type VI Cas_2 cut poly U ssRNA reporter with the maximum efficiency followed by the determined ssRNA reporter. Type VI Cas_2 complex was not able to cut poly A ssRNA reporter nor dinucleotide ssRNA reporter. Experiments were performed in 40 μL reaction volume containing 1× Binding Buffer (50 mM Potassium Acetate, 20 mM Tris-acetate, 10 mM Magnesium Acetate, 100 μg/ml BSA, pH 7.9), Type VI Cas_2 protein complexed to a final concentration of 150 nM Type VI Cas_2: 75 nM sgRNA: 20 nM activator (75 nt. ssRNA) and 150 nM of each of the aforementioned ssRNA FAMQ reporter substrates. Reactions were incubated at 37° C. in a Synergy H1 microplate reader (Bio-Tek) for 180 minutes as the endpoint time (ssRNA FQ substrates=λex: 485 nm; λem: 538 nm). Non-template negative control (NTC) fluorescence values were calculated from reactions carried out in the absence of ssRNA Hanta target. Results are shown in FIG. 96.

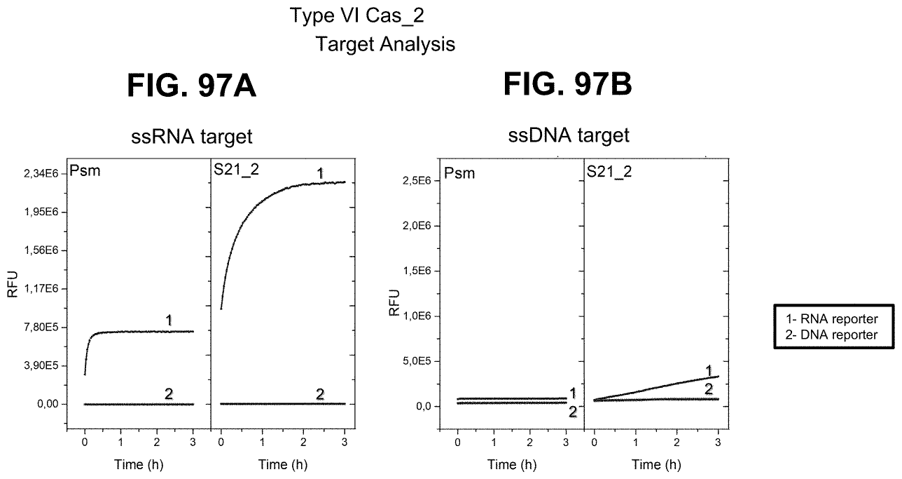

FIGS. 97A-97B shows the collateral activity for Type VI Cas_2 protein complexes using ssRNA and ssDNA substrates. FIGS. 97A-97B shows collateral activity for Type VI Cas_2 protein complex using as specific targets single-stranded RNA (IDT primer) and (B) single-stranded DNA (IDT primer). The specificity of trans-cleavage activity for ssRNA or ssDNA was tested using customized ssRNA (/56-FAM/rUrUrUrUrUrUrU/3IABkFQ/for Type VI Cas_2 and/56-FAM/rArArArArArArA/3IABkFQ/for Psm control) and customized ssDNA FAM/AAATTTCCCGGG/3IABkFQ (SEQ ID NO: 145), FAM/ATACAGAGTGCG/3IABkFQ (SEQ ID NO: 143), FAM/TATGTCTCACGC/3IABkFQ (SEQ ID NO: 144) from IDT (Integrated DNA Technologies, Inc.) as reporters. Results showed that Type VI Cas_2 was able to cut ssRNA reporter but not ssDNA reporter when using ssRNA as target. On the other hand, Type VI Cas_2 was able to cut a little of ssRNA reporter after 3 h but not ssDNA reporter when using ssDNA as target. The reaction was initiated by preparing complexes to a final concentration of 100 nM Type VI Cas_2: 75 nM sgRNA: 10 nM ssRNA (75 nt.) or ssDNA (60 nt.) activator in a solution containing 1× Binding Buffer (50 mM NaCl, 10 mM Tris-HCl, 1 mM DTT, 100 g/ml BSA, 10 mM of MgCl₂ and/or 10 nM MnCl₂, pH 7.9) and 250 nM ssRNA or ssDNA FAMQ reporter substrates in 40 μL reaction volume. Reactions were incubated at 30° C. in a Synergy H1 microplate reader (Bio-Tek) for 180 minutes as the endpoint time (ssRNA or ssDNA FQ substrates=λex: 485 nm; λem: 538 nm). Non-template negative control (NTC) fluorescence values were calculated from reactions carried out in the absence of ssRNA Hanta target. Results are shown in FIGS. 97A-97B.

### Example 8: Characterization of Type VI Cas_4

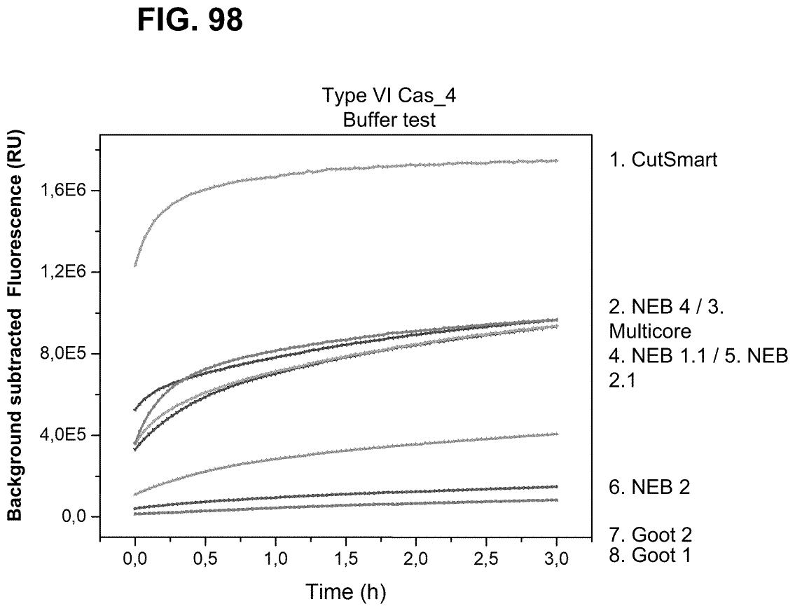

FIG. 98 Collateral activity of Type VI Cas_4 protein complex in different buffer solutions. The efficiency of trans-cleavage activity of Type VI Cas_4 protein was tested in different buffer solutions using RNaseAlert™ substrate from IDT (Integrated DNA Technologies, Inc.) as a reporter. The buffer solutions analyzed included: 1. CutSmart buffer from NEB (50 mM Potassium Acetate, 20 mM Tris-acetate, 10 mM Magnesium Acetate, 100 μg/ml BSA, pH 7.9); 2. NEB 4 buffer from NEB (50 mM Potassium Acetate, 20 mM Tris-acetate, 10 mM Magnesium Acetate, 1 mM DTT, pH 7.9); 3. NEB 1.1 buffer from NEB (10 mM Bis-Tris-Propane-HCl, 10 mM MgCl₂, 100 μg/ml BSA, pH 7); 4. Multicore buffer from Promega (25 mM Tris-acetate, 100 mM Potassium Acetate, 10 mM Magnesium Acetate, 1 mM DTT, pH 7.5); 5. NEB 2.1 buffer from NEB (10 mM Tris-HCl, 50 mM NaCl, 10 mM MgCl2, 100 μg/ml BSA, pH 7.9); 6. NEB 2 buffer from NEB (10 mM Tris-HCl, 50 mM NaCl, 10 mM MgCl2, 1 mM DTT, pH 7.9); 7. Goot 2 buffer (40 mM Tris-HCl, 60 mM NaCl, 6 mM MgCl2, pH 7.3) and 8. Goot 1 buffer (20 mM HEPES, 60 mM NaCl, 6 mM MgCl₂, pH 6.8). Reaction in CutSmart buffer demonstrated the best activity, evidenced for the highest fluorescence values. The protein also showed activity in NEB 4, Multicore, NEB 1.1 and NEB 2.1 buffers and to a lesser extent in NEB 2 buffer. The reaction was initiated by preparing complexes to a final concentration of 250 nM Type VI Cas_4: 125 nM sgRNA: 20 nM activator (31 nt. ssRNA from Synthego) and 150 nM of RNaseAlert reporter substrate, in each of the aforementioned buffer solutions in a 40 μl reaction at 30° C. Reactions were incubated in a Synergy H1 microplate reader (Bio-Tek) for 180 minutes with fluorescence measurements taken every 2 minutes (ssRNA FQ substrates=λex: 485 nm; Xem: 538 nm). Non-template negative control (NTC) fluorescence values were calculated from reactions carried out in the absence of ssRNA Hanta target. Results are shown in FIG. 98.

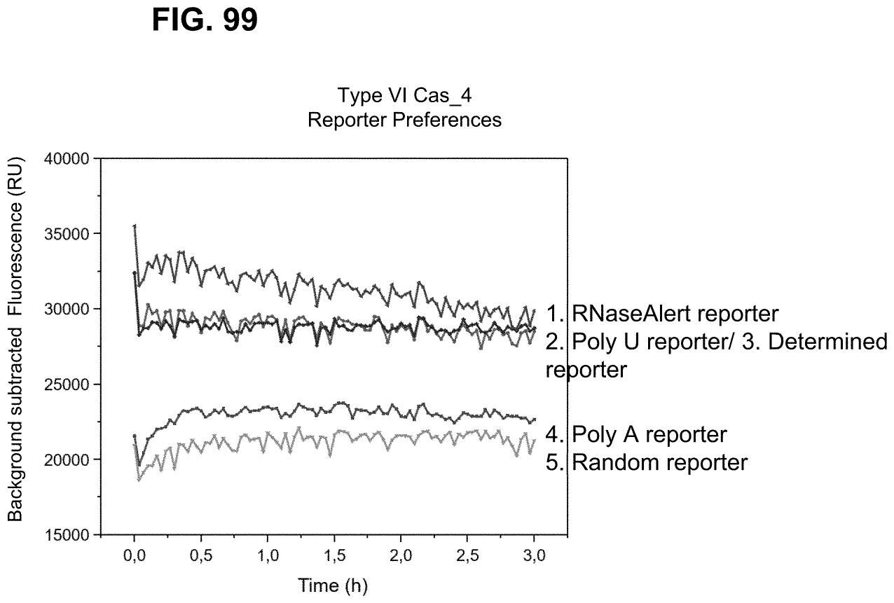

FIG. 99 shows the results from the collateral activity of the Type VI Cas_4 protein complex for different customized ssRNA reporter substrates. Efficiency of trans-cleavage activity for different customized ssRNA reporters from IDT (Integrated DNA Technologies, Inc.). The ssRNA reporters analyzed were: poly A (/56-FAM/rArArArArArArA/3IABkFQ/), poly U (/56-FAM/rUrUrUrUrUrUrU/3IABkFQ/), random (/56-FAM/rUrNrNrNrNrNrN/3IABkFQ/), determined (/56-FAM/rUrUrArUrUrArUrU/3IABkFQ/) and RNaseAlert substrate from IDT. The results showed that Type VI Cas_4 was able to cut all the reporter substrates tested, with a higher preference for RNaseAlert, followed by the determined and poly U ssRNA reporters. Experiments were performed in 40 μL reaction volume containing 1× Binding Buffer (50 mM Potassium Acetate, 20 mM Tris-acetate, 10 mM Magnesium Acetate, 100 μg/ml BSA, pH 7.9), Type VI Cas_4 protein complexed to a final concentration of 250 nM Type VI Cas_4: 125 nM sgRNA: 20 nM activator (75 nt. ssRNA) and 250 nM of each of the aforementioned ssRNA FAMQ reporter substrates. Reactions were incubated at 30° C. in a Synergy H1 microplate reader (Bio-Tek) microplate reader (Molecular Devices) for 180 minutes as the endpoint time (ssRNA FQ substrates=λex: 485 nm; λem: 538 nm). Non-template negative control (NTC) fluorescence values were calculated from reactions carried out in the absence of ssRNA Hanta target. Results are shown in FIG. 99.

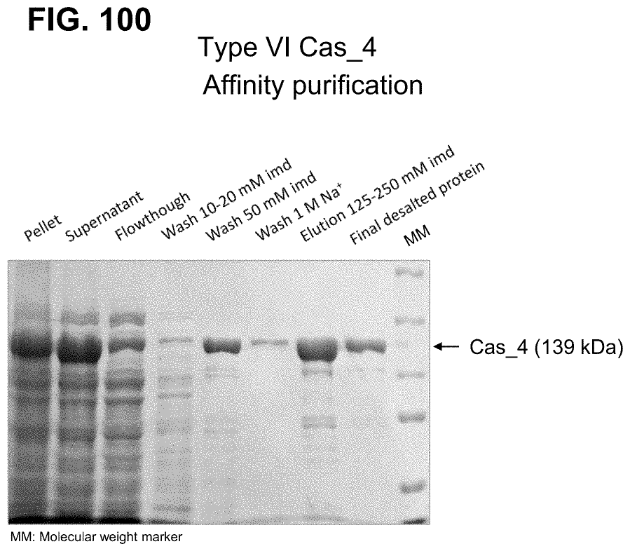

FIG. 100 shows 10% SDS-PAGE analysis of Type VI Cas_2 purification. The Type VI Cas_4 protein was purified as recombinant protein expressed in E. coli NiCo21 (DE3) cells (NEB #C2529H) harboring the pET28a/Type VI Cas_4-H6X expression plasmid by growing in LB broth culture medium at 37° C. followed by induction of expression overnight at 24° C. in presence of 0.25 mM IPTG. Cells were disrupted by sonication prior to chromatographic purification. Recombinant protein was purified using a His-Trap HP (Ni-NTA GE Healthcare) followed by a HiPrep™ 26/10 desalting column (GE Healthcare) where the protein was desalted into storage buffer containing 50 mM Tris-HCl pH 8.0, 200 mM NaCl, 1 mM DTT and 20 mM MgCl₂. Protein purity was controlled by Coomassie blue staining after SDS-PAGE on a 10% polyacrylamide gel. Protein concentrations were determined by UV spectroscopy and Qubit protein assay (Invitrogen). Purified proteins were stored at −80° C.

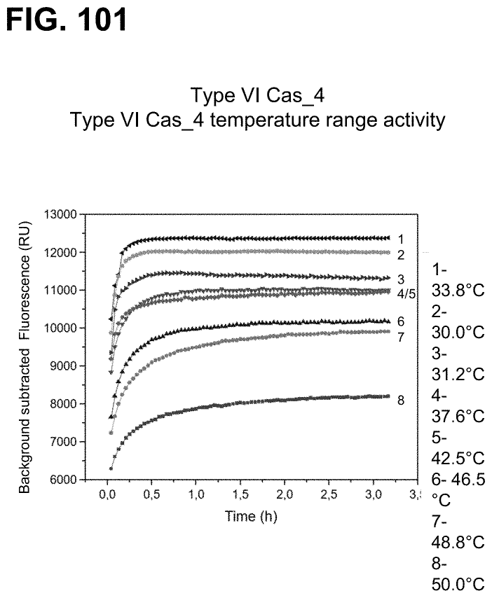

FIG. 101 shows collateral activity of the Type VI Cas_4 protein complex in a temperature curve (30° C.-50° C.). The efficiency of trans-cleavage activity at different temperatures was tested using RNaseAlert™ substrate from IDT (Integrated DNA Technologies, Inc.) as a reporter. The temperatures analyzed in a time course cleavage were: 30.0° C., 31.2° C., 33.8° C., 37.6° C., 42.5° C., 46.5° C., 48.8° C. and 50.0°. The results showed that Type VI Cas_4 was able to cleave the ssRNA reporter more efficiently in the range between 30−42.5° C., with an optimal activity at 33.8° C. Detection assay was performed at the different temperatures using Type VI Cas_4 complexes to a final concentration of 250 nM Type VI Cas_4: 125 nM sgRNA: 20 nM activator (75 nt. ssRNA from Synthego) in a solution containing 1× Binding Buffer (50 mM Potassium Acetate, 20 mM Tris-acetate, 10 mM Magnesium Acetate, 100 μg/ml BSA, pH 7.9) and 150 nM of ssRNA FAMQ reporter substrate in a 40 μl reaction. Reactions were incubated in a qPCR (Bio-Rad) for 180 minutes with fluorescence measurements taken every 2.5 minutes (ssRNA FQ substrates=λex: 485 nm; λem: 538 nm). Non-template negative control (NTC) fluorescence values were calculated from reactions carried out in the absence of ssRNA Hanta target.

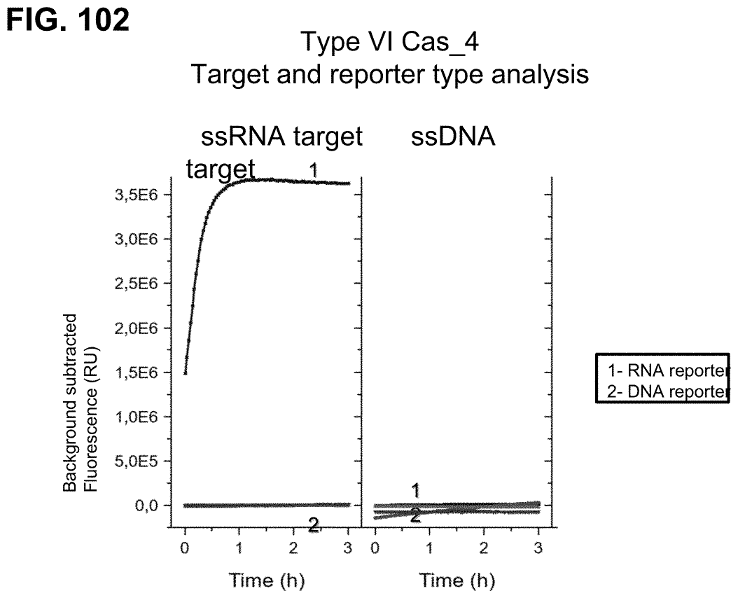

FIG. 102 depicts the collateral activity for Type VI Cas_4protein complex using ssRNA and ssDNA substrates. Collateral activity for Type VI Cas_4 protein complex using as specific targets single-stranded RNA (IDT primer) and (B) single-stranded DNA (Macrogen primer). The specificity of trans-cleavage activity for ssRNA or ssDNA was tested using RNaseAlert substrate or customized ssDNA FAM/AAATTTCCCGGG/3IABkFQ (SEQ ID NO: 145), FAM/ATACAGAGTGCG/3IABkFQ (SEQ ID NO: 143), FAM/TATGTCTCACGC/3IABkFQ (SEQ ID NO: 144) from IDT (Integrated DNA Technologies, Inc.) as reporters. Results showed that Type VI Cas_4 was able to cut ssRNA reporter but not ssDNA reporter when using ssRNA as target. On the other hand, Type VI Cas_4 was not able to cut ssRNA nor ssDNA reporters when using ssDNA as target. The reaction was initiated by preparing complexes to a final concentration of 250 nM Type VI Cas_4: 125 nM sgRNA: 10 nM ssRNA (75 nt.) or ssDNA (60 nt.) activator in a solution containing 1× Binding Buffer (50 mM NaCl, 10 mM Tris-HCl, 1 mM DTT, 100 g/ml BSA, 10 mM of MgCl₂ and/or 10 nM MnCl₂, pH 7.9) and 250 nM ssRNA or ssDNA FAMQ reporter substrates in 40 μL reaction volume. Reactions were incubated at 37° C. in a Synergy H1 microplate reader (Bio-Tek) for 180 minutes as the endpoint time (ssRNA or ssDNA FQ substrates=λex: 485 nm; λem: 538 nm). Non-template negative control (NTC) fluorescence values were calculated from reactions carried out in the absence of ssRNA Hanta target.

1. An engineered system comprising:
a. a Class 2 CRISPR-Cas endonuclease or a nucleic acid encoding the endonuclease, wherein the Class 2 CRISPR-Cas endonuclease is:
i. a Class 2 Type II CRISPR-Cas endonuclease comprising at least one of the RuvC sequences of Table 7, or a sequence comprising at least 60% sequence identity thereto;
ii. a Class 2 Type V CRISPR-Cas endonuclease comprising at least one of the RuvC sequences of Table 1, or a sequence comprising at least 60% sequence identity thereto; or
iii. a Class 2 Type VI CRISPR-Cas endonuclease comprising at least one of the HEPN sequences of Table 4, or a sequence comprising at least 60% sequence identity thereto, and
b. a gRNA or a nucleic acid encoding the gRNA, wherein the gRNA and the Class 2 CRISPR-Cas endonuclease do not naturally occur together, wherein the gRNA is capable of hybridizing to a target sequence in a target DNA or RNA, and the gRNA is capable of forming a complex with the Class 2 CRISPR-Cas endonuclease.

2. The system of claim 1, comprising:
a. a Class 2 Type II CRISPR-Cas endonuclease; and
b. a Class 2 Type II CRISPR-Cas gRNA.

3. The system of claim 1, comprising:
a. a nucleic acid encoding the Class 2 Type II CRISPR-Cas endonuclease; and
b. a nucleic acid encoding the Class 2 Type II CRISPR-Cas endonuclease gRNA.

4. The system of claim 1, wherein the gRNA is a single-molecule gRNA.

5. The Class 2 Type II CRISPR-Cas endonuclease containing system of claim 1, wherein the gRNA is a dual-molecule gRNA.

6. The system of claim 1, wherein the Class 2 CRISPR-Cas endonuclease is a Class 2 Type II CRISPR-Cas endonuclease comprising at least one of the RuvC or HNH sequences of Table 7, or a sequence comprising at least 60% sequence identity thereto, or is a Class 2 Type V CRISPR-Cas endonuclease comprising at least one of the RuvC or HNH sequences of Table 1, or a sequence comprising at least 60% sequence identity thereto, and the target is target DNA.

7. The system of claim 1, wherein the Class 2 CRISPR-Cas endonuclease is a Class 2 Type VI CRISPR-Cas endonuclease comprising at least one of the HEPN sequences of Table 4, or a sequence comprising at least 60% sequence identity thereto, and the target is target RNA.

8. The system of claim 7, wherein the target RNA is mRNA, tRNA, rRNA, miRNA, or siRNA.

9. The system of claim 1, wherein the Class 2 Type II CRISPR-Cas endonuclease comprises any one of SEQ ID NOS: 16-19, or a sequence comprising at least 60% sequence identity thereto.

10. The system of claim 1, wherein the Class 2 Type V CRISPR-Cas endonuclease comprises any one of SEQ ID NOS: 1-7 or 20, or a sequence comprising at least 60% sequence identity thereto.

11. The system of claim 1, wherein the Class 2 Type VI CRISPR-Cas endonuclease comprises any one of SEQ ID NOS: 8-15, or a sequence comprising at least 60% sequence identity thereto.

12. An engineered single-molecule gRNA, comprising:
a. a targeter-RNA comprising a spacer sequence that is capable of hybridizing with a target sequence in a target DNA; and
b. an activator-RNA that is capable of hybridizing with the targeter-RNA to form a double-stranded RNA duplex,
wherein the targeter-RNA and the activator-RNA are covalently linked to one another, wherein the single-molecule gRNA is capable of forming a complex with a Class 2 Type II CRISPR-Cas endonuclease, wherein hybridization of the spacer sequence to the target sequence is capable of targeting the endonuclease to a target DNA, and wherein the Class 2 Type II CRISPR-Cas endonuclease comprises at least one of the RuvC or HNH sequences of Table 7, or a sequence comprising at least 60% sequence identity thereto.

13. The gRNA of claim 12, wherein the Class 2 Type II CRISPR-Cas endonuclease comprises any one of SEQ ID NOS: 16-19, or a sequence comprising at least 60% sequence identity thereto.

14. The gRNA of claim 12, wherein the targeter-RNA and the activator-RNA are arranged in a 5′ to 3′ orientation.

15. The gRNA of claim 12, wherein the activator-RNA and the targeter-RNA are arranged in a 5′ to 3′ orientation.

16. The gRNA of claim 12, wherein the targeter-RNA and the activator-RNA are covalently linked to one another via a linker.

17. The gRNA of claim 12, wherein the single-molecule gRNA comprises one or more sequence modifications compared to a sequence of a corresponding wild type tracrRNA and/or crRNA.

18. The gRNA of claim 12, wherein the targeter-RNA comprises a spacer sequence of about 10-50 nucleotides that have 100% complementarity to a sequence in the target DNA.

19. The gRNA of claim 12, wherein the targeter-RNA comprises a spacer sequence of about 10-50 nucleotides that have less than 100% complementarity to a sequence in the target DNA.

20-61. (canceled)

62. An endonuclease comprising an amino acid sequence with 30%-99.5% homology to any one of SEQ ID NOs: 1-20.

63. A composition comprising the endonuclease of claim 62.

64. The composition of claim 63, further comprising a pharmaceutically acceptable carrier, a nucleic acid stabilizing buffer and/or or an endonuclease stabilizing buffer.

65. The composition of claim 63, wherein the endonuclease is lyophilized, and the composition further comprises any one or more of a labeled detector, a reverse transcriptase enzyme, and reagents for loop-mediated isothermal amplification.

66. A DNA polynucleotide comprising a nucleotide sequence that encodes the endonuclease of claim 62.

67. A recombinant expression vector comprising the DNA polynucleotide of claim 66.

68. The recombinant expression vector of claim 67, wherein the nucleotide sequence encoding the endonuclease is operably linked to a promoter.

69. A host cell comprising the DNA polynucleotide of claim 66.

70. (canceled)

71. A composition comprising the engineered system of claim 1, and a nucleic acid stabilizing buffer and/or an endonuclease stabilizing buffer.

72-73. (canceled)

74. A DNA polynucleotide comprising a nucleotide sequence that encodes the single molecule gRNA of claim 12.

75. A recombinant expression vector comprising the DNA polynucleotide of claim 74.

76. The recombinant expression vector of claim 75, wherein the nucleotide sequence encoding the single molecule gRNA is operably linked to a promoter.

77. A host cell comprising the DNA polynucleotide of claim 74.

78. A kit comprising one or more components of the engineered system of claim 1.

79. The kit of claim 78, wherein one or more components are lyophilized.

80. The kit of claim 78, wherein the one or more components further comprise a labeled reporter and a gRNA directed to SARS-CoV-2.
